(19) United States
(12) Patent Application Publication
(10) Pub. No.: US 2023/0079440 A1
(43) Pub. Date: Mar. 16, 2023

(54) COMPOSITIONS AND METHODS FOR GENOME ENGINEERING

(71) Applicant: Sangamo Therapeutics, Inc., Richmond, CA (US)

(72) Inventor: Ivan Krivega, Richmond, CA (US)

(21) Appl. No.: 17/773,509

(22) PCT Filed: Oct. 30, 2020
(86) PCT No.: PCT/US2020/058370

§ 371 Date: Apr. 29, 2022

Related U.S. Application Data
(60) Provisional application No. 62/929,523, filed on Nov. 1, 2019.

(51) Int. Cl. C07K 14/61; C12N 15/86; C12N 9/22

(52) U.S. Cl. CPC C07K 14/61; C12N 15/86; C12N 9/22; C12N 2750/14143; C12N 2310/20

(57) ABSTRACT

The present disclosure provides push-pull donor constructs and methods of uses thereof in genome engineering.

### CROSS-REFERENCE TO RELATED APPLICATIONS

This application claims priority and benefit from U.S. Provisional Application No. 62/929,523, filed Nov. 1, 2019, the contents of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Oct. 27, 2020, is named 000222-0009-WO1_SL.txt and is 392,177 bytes in size.

### BACKGROUND

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus.

These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), using the CRISPR/Cas system with an engineered crRNA/tracr RNA (single guide RNA′) to guide specific cleavage and/or using nucleases based on the Argonaute system (e.g., from T. thermophilus, known as ‘TtAgo’, (Swarts, et al. (2014) Nature 507(7491): 258-261).

Targeted cleavage using one of the above-mentioned nuclease systems can be exploited to insert a nucleic acid into a specific target location using either HDR or NHEJ-mediated processes. However, conventional methods for inserting a nucleic acid into a target location in certain cell types (e.g., cardiomyocytes, medium spiny neurons, primary hepatocytes, embryonic stem cells, induced pluripotent stem cells and muscle cells) using NHEJ are not efficient because only half of the integration events are productive in that the donor nucleic acid is inserted in the correct orientation.

Thus, there remains a need for compositions and methods for genome engineering of cells of interest that are more efficient.

### SUMMARY

The present disclosure provides donor constructs configured in a “push-pull” orientation to allow for improved expression of a therapeutic protein. These “push-pull” donor constructs are capable of integrating into a target genome with high precision and efficiency and are therefore useful in methods for treating e.g., genetic disorders in a subject, the method comprising modifying a target nucleotide sequence in the genome of a cell. Thus, a first aspect of the disclosure provides a polynucleotide construct comprising in 5′ to 3′ orientation:
a. a first Inverted Terminal Repeat (ITR) nucleotide sequence;
b. a first nucleotide sequence encoding a first polypeptide;
c. a second nucleotide sequence encoding a second polypeptide; and
d. a second ITR nucleotide sequence;
   wherein the first nucleotide sequence encoding a first polypeptide is oriented tail-to-tail to the second nucleotide sequence encoding a second polypeptide; and wherein the first nucleotide sequence and the second nucleotide sequence encode a polypeptide having the same amino acid sequence.

In some embodiments, the polynucleotide construct of the disclosure, further comprises:
e. a first splice acceptor sequence operatively linked to the first nucleotide sequence encoding the first polypeptide; and
f. a second splice acceptor sequence operatively linked to the second nucleotide sequence encoding the second polypeptide.
   In some embodiments, each of said first splice acceptor sequence and second splice acceptor sequence is independently selected from a Factor 9 Splice Acceptor (F9SA), a CFTR Splice acceptor, a COL5A2 Splice acceptor, a NF1 Splice Acceptor, a MLH1 Splice Acceptor, and an Albumin (ALB) Splice Acceptor.

In some embodiments, the polynucleotide construct further comprises:
g. a first polyadenylation (polyA) signal sequence operatively linked to the nucleotide sequence encoding the first polypeptide; and
h. a second polyadenylation (polyA) signal sequence operatively linked to the nucleotide sequence encoding the second polypeptide.
   In some embodiments, the first polyA signal sequence is selected from a human Growth Hormone (hGH) polyA signal, a bovine Growth Hormone (bGH) polyA signal, a SV40 polyA signal, and a rbGlob polyA signal. In some embodiments, the second polyA signal sequence is selected from a human Growth Hormone (hGH) polyA signal, a bovine Growth Hormone (bGH) polyA signal, a SV40 polyA signal, and a rbGlob polyA signal.

In some embodiments, the nucleotide sequence encoding the first polypeptide or the nucleotide sequence encoding the second polypeptide encodes a therapeutic polypeptide. In some embodiments, the therapeutic polypeptide is selected from the group consisting of iduronate-2-sulphatase (IDS), alpha-L-iduronidase (IDUA), alpha-D-mannosidase, N-aspartyl-beta-glucosaminidase, lysosomal acid lipase, cystinosin, lysosomal associated membrane protein 2, alpha-galactosidase A, acid ceramidase, alpha fucosidase, cathepsin A, acid beta-glucocerebrosidase, beta galactosidase, beta hexosaminidase A, beta hexosaminidase B, beta hexosaminidase, GM2 ganglioside activator, GLcNAc-1-phosphotransferase, Beta-galactosylceramidase, arylsulfatase A, heparan N-sulfatase, alpha-N-acetylglucosaminidase, acetyl CoA:alpha-glucosaminide acetyltransferase, N-acetyl glucosamine-6-sulfatase, arylsulfatase B, beta-glucuronidase, hyaluronidase, neuraminidase, mucolipin-1, formylglycine-generating enzyme, palmitoyl-protein thioesterase 1, tripeptidyl peptidase 1, CLN3 protein, cysteine string protein alpha, CLN5 protein, CLN6 protein, CLN7 protein, CLN8 protein, acid sphingomyelinase, NPC 1, NPC 2, phenylalanine hydroxylase, acid alpha-glucosidase, cathepsin K, sialin, alpha-N-acetylgalactosaminidase, glucose-6-phosphatase, solute carrier family 37 member 4, argininosuccinate synthase 1, solute carrier family 25 member 13, and ornithine transcarbamylase.

In some embodiments, the nucleotide sequence encoding the first polypeptide is codon diversified. In some embodiments, the nucleotide sequence encoding the second polypeptide is codon diversified. In some embodiments, each of the nucleotide sequence encoding the first polypeptide and the nucleotide sequence encoding the second polypeptide is each independently codon diversified.

In some embodiments, the nucleotide sequence encoding the first polypeptide comprises the nucleotide sequence set forth in any one of SEQ ID NOs: 184-193. In some embodiments, the nucleotide sequence encoding the second polypeptide comprises the nucleotide sequence set forth in any one of SEQ ID NOs: 184-193. In some embodiments, the polynucleotide construct comprises the nucleotide sequence set forth in any one of SEQ ID NOs: 173-176.

A second aspect of the disclosure provides a vector comprising the polynucleotide construct of the disclosure. In some embodiments, the vector is an adeno-associated viral (AAV) vector. In some embodiments, the AAV is selected from the group consisting of AAV-MeCP2, AAV1, AAV2, AAV3, AAV4, AAV5, AAV6, AAV8, AAV8.2, AAV9, Dual AAV9, AAVrh8, AAVrh10, AAHrh43, AAVhu37, AAV2/8, AAV2/5, and AAV2/6.

A third aspect of the disclosure provides a cell comprising the polynucleotide construct or the vector of the disclosure. In some embodiments, the cell is a eukaryotic cell. In some embodiments, the cell is a mammalian cell. In some embodiments, the cell is a stem cell. In some embodiments, the cell is a human cell. In some embodiments, the cell is a non-dividing cell. In some embodiments, the cell is a hepatocyte.

In some embodiments, the cell further comprises a polynucleotide encoding a nuclease. In some embodiments, the cell further comprises a first polynucleotide encoding a first zinc finger nuclease (ZFN) and a second polynucleotide encoding a second zinc finger nuclease (ZFN). In some embodiments, the cell further comprises a first vector comprising a first polynucleotide encoding a first zinc finger nuclease (ZFN) and a second vector comprising a second polynucleotide encoding a second zinc finger nuclease (ZFN). In some embodiments, the cell further comprises a polynucleotide encoding one or more zinc finger nucleases (ZFN). In some embodiments, the cell further comprises a vector comprising a polynucleotide encoding one or more zinc finger nucleases (ZFN). In some embodiments, the zinc finger nuclease is a 2-in-1 zinc finger nuclease.

A fourth aspect of the disclosure provides a pharmaceutical composition comprising the polynucleotide construct of the disclosure; and a pharmaceutically acceptable carrier. In some embodiments, the pharmaceutical composition further comprises a first polynucleotide encoding a first zinc finger nuclease (ZFN) and a second polynucleotide encoding a second zinc finger nuclease (ZFN). In some embodiments, the pharmaceutical composition comprises a first vector comprising a first polynucleotide encoding a first zinc finger nuclease (ZFN) and a second vector comprising a second polynucleotide encoding a second zinc finger nuclease (ZFN). In some embodiments, the pharmaceutical composition further comprises a polynucleotide encoding one or more zinc finger nucleases (ZFN). In some embodiments, the pharmaceutical composition further comprises a vector comprising a polynucleotide encoding one or more zinc finger nucleases (ZFN). In some embodiments, the zinc finger nuclease in the pharmaceutical composition of the disclosure is a 2-in-1 zinc finger nuclease.

In some embodiments, the ratio of the polynucleotide encoding the first zinc finger nuclease: the polynucleotide encoding the second zinc finger: the polynucleotide of the disclosure is 1:1:8. In some embodiments, the ratio of the polynucleotide encoding the first zinc finger nuclease: the polynucleotide encoding the second zinc finger: the polynucleotide of the disclosure is 1:1:4. In some embodiments, the ratio of the polynucleotide encoding the first zinc finger nuclease: the polynucleotide encoding the second zinc finger: the polynucleotide of the disclosure is 1:1:2. In some embodiments, the ratio of the polynucleotide encoding the first zinc finger nuclease: the polynucleotide encoding the second zinc finger: the polynucleotide of the disclosure is 3:3:4. In some embodiments, the ratio of the vector comprising the first polynucleotide encoding the first zinc finger nuclease: the vector comprising the polynucleotide encoding the second zinc finger: the vector of the disclosure is 1:1:8. In some embodiments, the ratio of the vector comprising the first polynucleotide encoding the first zinc finger nuclease: the vector comprising the polynucleotide encoding the second zinc finger: the vector of the disclosure is 1:1:4. In some embodiments, the ratio of the vector comprising the first polynucleotide encoding the first zinc finger nuclease: the vector comprising the polynucleotide encoding the second zinc finger: the vector of the disclosure is 1:1:2. In some embodiments, the ratio of the vector comprising the first polynucleotide encoding the first zinc finger nuclease: the vector comprising the polynucleotide encoding the second zinc finger: the vector of the disclosure is 3:3:4.

In some embodiments, the ratio of the polynucleotide encoding the 2-in-1 zinc finger nuclease: the polynucleotide construct of the disclosure is 1:4. In some embodiments, the ratio of the polynucleotide encoding the 2-in-1 zinc finger nuclease: the polynucleotide construct of the disclosure is 1:2. In some embodiments, the ratio of the polynucleotide encoding the 2-in-1 zinc finger nuclease: the polynucleotide construct of the disclosure is 1:1. In some embodiments, the ratio of the polynucleotide encoding the 2-in-1 zinc finger nuclease: the polynucleotide construct of the disclosure is 3:2. In some embodiments, the ratio of the vector comprising the 2-in-1 zinc finger nuclease: the vector of the disclosure is 1:4. In some embodiments, the ratio of the vector comprising the 2-in-1 zinc finger nuclease: the vector of the disclosure is 1:2. In some embodiments, the ratio of the vector comprising the 2-in-1 zinc finger nuclease: the vector of the disclosure is 1:1. In some embodiments, the ratio of the vector comprising the 2-in-1 zinc finger nuclease: the vector of the disclosure is 3:2.

In some embodiments, wherein the composition is formulated for intravenous, intramuscular, subcutaneous, or intrathecal administration.

A fifth aspect of the disclosure provides a method for modifying the genome of a cell. In some embodiments, the method for modifying the genome of a cell comprises introducing into a cell an effective amount of the polynucleotide construct of the disclosure. In some embodiments, the method for modifying the genome of a cell comprises introducing into a cell an effective amount of the vector of the disclosure. In some embodiments, the method for modifying the genome of a cell comprises introducing into a cell an effective amount of the pharmaceutical composition of the disclosure.

A sixth aspect of the disclosure provides a method for integrating an exogenous nucleotide sequence into a target nucleotide sequence of a cell. In some embodiments, the method for integrating an exogenous nucleotide sequence into a target nucleotide sequence of a cell comprises introducing into a cell an effective amount of the polynucleotide construct of the disclosure. In some embodiments, the method for integrating an exogenous nucleotide sequence into a target nucleotide sequence of a cell comprises introducing into a cell an effective amount of the vector of the disclosure. In some embodiments, the method for integrating an exogenous nucleotide sequence into a target nucleotide sequence of a cell comprises introducing into a cell an effective amount of the pharmaceutical composition of the disclosure.

A seventh aspect of the disclosure provides a method for disrupting a target nucleotide sequence in a cell. In some embodiments, the method for disrupting a target nucleotide sequence in a cell comprises introducing into a cell an effective amount of the polynucleotide construct of the disclosure. In some embodiments, the method for disrupting a target nucleotide sequence in a cell comprises introducing into a cell an effective amount of the vector of the disclosure. In some embodiments, the method for disrupting a target nucleotide sequence in a cell, comprises introducing into a cell an effective amount of the pharmaceutical composition of the disclosure.

An eighth aspect of the disclosure provides a method for treating a disorder in a subject. In some embodiments, the method for treating a disorder in a subject comprises modifying a target nucleotide sequence in the genome of a cell of said subject by introducing into the cell an effective amount of the polynucleotide construct of the disclosure. In some embodiments, the method for treating a disorder in a subject comprises modifying a target nucleotide sequence in the genome of a cell of said subject by introducing into the cell an effective amount of the vector of the disclosure. In some embodiments, the method for treating a disorder in a subject comprises modifying a target nucleotide sequence in the genome of a cell of said subject by introducing into the cell an effective amount of the pharmaceutical composition of the disclosure.

In some embodiments, the methods of the disclosure further comprise introducing into the cell an effective amount of a first polynucleotide encoding a first zinc finger nuclease (ZFN) and a second polynucleotide encoding a second zinc finger nuclease (ZFN). In some embodiments, the methods of the disclosure further comprise introducing into the cell an effective amount of a first vector comprising a first polynucleotide encoding a first zinc finger nuclease (ZFN) and a second vector comprising a second polynucleotide encoding a second zinc finger nuclease (ZFN). In some embodiments, the methods of the disclosure further comprise introducing into the cell an effective amount of a polynucleotide encoding one or more zinc finger nucleases (ZFN). In some embodiments, the methods of the disclosure further comprise introducing into the cell an effective amount of a vector comprising a polynucleotide encoding one or more zinc finger nucleases (ZFN). In some embodiments, the zinc finger nuclease used in the methods of the disclosure is a 2-in-1 zinc finger nuclease.

In some embodiments, upon integration of the polynucleotide construct of the disclosure into the genome of the cell, the first nucleotide sequence encoding the first polypeptide is expressed. In some embodiments, upon integration of the polynucleotide construct of the disclosure into the genome of the cell, the second nucleotide sequence encoding the second polypeptide is expressed.

In some embodiments, the disorder is selected from the group consisting of a, a genetic disorder, an infectious disease, an acquired disorder, and a cancer. In some embodiments, the genetic disorder is selected from the group consisting of achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangiectasia, barth syndrome, beta-thalassemia, blue rubber bleb nevus syndrome, canavan disease, chronic granulomatous diseases (CGD), citrullinemia, cri du chat syndrome, cystic fibrosis, dercum's disease, ectodermal dysplasia, Fabry disease, fanconi anemia, fibrodysplasia ossificans progressive, fragile X syndrome, galactosemis, Gaucher's disease, generalized gangliosidoses (e.g., GM1), GSD (e.g., GSD1a) hemochromatosis, the hemoglobin C mutation in the 6th codon of beta-globin (HbC), hemophilia, Hunter syndrome, Huntington's disease, Hurler Syndrome, hypophosphatasia, Klinefelter syndrome, Krabbes Disease, Langer-Giedion Syndrome, leukocyte adhesion deficiency (LAD, OMIM No. 116920), leukodystrophy, long QT syndrome, lipoprotein lipase deficiency, Marfan syndrome, Moebius syndrome, mucopolysaccharidosis (MPS), nail patella syndrome, nephrogenic diabetes insipdius, neurofibromatosis, Neimann-Pick disease, ornithine transcarbamylase (OTC) deficiency, osteogenesis imperfecta, phenylketonuria (PKU), Pompe disease, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, and X-linked lymphoproliferative syndrome (XLP, OMIM No. 308240).

In some embodiments, the genetic disorder is a lysosomal storage disease. In some embodiments, the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/J/A), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease. In some embodiments, the lysosomal storage disease is selected from MPSI and MPSII. In some embodiments, the lysosomal storage disease is selected from the group consisting of MPS I—Hurler Syndrome, MPS I—Scheie Syndrome, and MPS I-Hurler-Scheie Syndrome. In some embodiments, the lysosomal storage disease is MPSII Hunter Syndrome.

In some embodiments, the infectious disease is selected from the group consisting of herpes simplex virus (HSV), such as HSV-1 and HSV-2, varicella zoster virus (VZV), Epstein-Barr virus (EBV), cytomegalovirus (CMV), human herpesvirus 6 (HHV-6), human herpesvirus 7 (HHV-7), hepatitis A virus (HAV), hepatitis B virus (HBV), hepatitis C virus (HCV), the delta hepatitis virus (HDV), hepatitis E virus (HEV), hepatitis G virus (HGV), Picornaviridae, Caliciviridae, Togaviridae, Flaviviridae, Coronaviridae, Reoviridae, Birnaviridae, Rhabodoviridae, Filoviridae, Paramyxoviridae, Orthomyxoviridae, Bunyaviridae, Arenaviridae, Retroviradae, lentiviruses, simian immunodeficiency virus (SIV), human papillomavirus (HPV), influenza virus and tick-borne encephalitis viruses.

In some embodiments, the vector is administered at a dose of about 1×10⁹ vg/kg to about 1×10¹⁷ vg/kg. In some embodiments, the vector is administered at a dose selected from the group consisting of about 5×10¹² vg/kg, about 1×10¹³ vg/kg, about 5×10¹³ vg/kg and about 1×10¹⁴ vg/kg. In some embodiments, the vector comprising the polynucleotide encoding one or more zinc finger nucleases is administered at a dose of about 1×10¹² vg/kg to about 1×10¹⁴ vg/kg.

A ninth aspect of the disclosure provides a method for correcting a disease-causing mutation in the genome of a cell. In some embodiments, the method for correcting a disease-causing mutation in the genome of a cell comprises modifying a target nucleotide sequence in the genome of the cell by introducing into the cell an effective amount of the polynucleotide construct of the disclosure. In some embodiments, the method for correcting a disease-causing mutation in the genome of a cell comprises modifying a target nucleotide sequence in the genome of the cell by introducing into the cell an effective amount of the vector of the disclosure. In some embodiments, the method for correcting a disease-causing mutation in the genome of a cell comprises modifying a target nucleotide sequence in the genome of the cell by introducing into the cell an effective amount of the pharmaceutical composition of the disclosure. In some embodiments, the method further comprises introducing into the cell an effective amount of a first polynucleotide encoding a first zinc finger nuclease (ZFN) and a second polynucleotide encoding a second zinc finger nuclease (ZFN). In some embodiments, the method further comprises introducing into the cell an effective amount of a first vector comprising a first polynucleotide encoding a first zinc finger nuclease (ZFN) and a second vector comprising a second polynucleotide encoding a second zinc finger nuclease (ZFN). In some embodiments, the method further comprises introducing into the cell an effective amount of a polynucleotide encoding one or more zinc finger nucleases (ZFN). In some embodiments, the method further comprises introducing into the cell an effective amount of a vector comprising a polynucleotide encoding one or more zinc finger nucleases (ZFN). In some embodiments, upon integration of the polynucleotide construct of the disclosure into the genome of the cell, the first nucleotide sequence encoding the first polypeptide is expressed. In some embodiments, upon integration of the polynucleotide construct of the disclosure into the genome of the cell, the second nucleotide sequence encoding the second polypeptide is expressed.

In some embodiments, the cell is a eukaryotic cell. In some embodiments, the cell is a mammalian cell. In some embodiments, the cell is a stem cell. In some embodiments, the cell is a human cell. In some embodiments, the cell is a non-dividing cell. In some embodiments, the cell is a hepatocyte. In some embodiments, the target nucleotide sequence is an endogenous locus.

A tenth aspect of the disclosure provides the use of a polynucleotide construct of the disclosure for the preparation of a medicament for treating a disease or disorder.

An eleventh aspect of the disclosure provides the use of a polynucleotide construct of the disclosure for the preparation of a medicament for modifying the genome of a cell.

A twelfth aspect of the disclosure provides the use of a polynucleotide construct of the disclosure for the preparation of a medicament for integrating a transgene into a target nucleotide sequence of a cell.

A thirteenth aspect of the disclosure provides the use of a polynucleotide construct of the disclosure for the preparation of a medicament for disrupting a target nucleotide sequence in a cell.

A fourteenth aspect of the disclosure provides the use of a polynucleotide construct of the disclosure for the preparation of a medicament for correcting a disease-causing mutation in the genome of a cell.

A fifteenth aspect of the disclosure provides the use of a polynucleotide construct of the disclosure for the preparation of a medicament for modifying a target nucleotide sequence in the genome of a cell.

A sixteenth aspect of the disclosure provides a polynucleotide construct of the disclosure, for use in treating a disease or disorder.

A seventeenth aspect of the disclosure provides a polynucleotide construct of the disclosure, for use in modifying the genome of a cell.

An eighteenth aspect of the disclosure provides a polynucleotide construct of the disclosure, for use in integrating a transgene into a target nucleotide sequence of a cell.

A nineteenth aspect of the disclosure provides a polynucleotide construct of the disclosure, for use in disrupting a target nucleotide sequence in a cell.

A twentieth aspect of the disclosure provides a polynucleotide construct of the disclosure, for use in correcting a disease-causing mutation in the genome of a cell.

A twenty-first aspect of the disclosure provides a polynucleotide construct of the disclosure, for use in modifying a target nucleotide sequence in the genome of a cell.

### BRIEF DESCRIPTION OF THE DRAWINGS

FIG. 1 shows a schematic of conventional non-homologous end joining (NHEJ) method of inserting a inserting a nucleic acid into a target location. This method results in only half of the integration events being productive (i.e., the nucleic acid is inserted in the correct orientation into the target site).

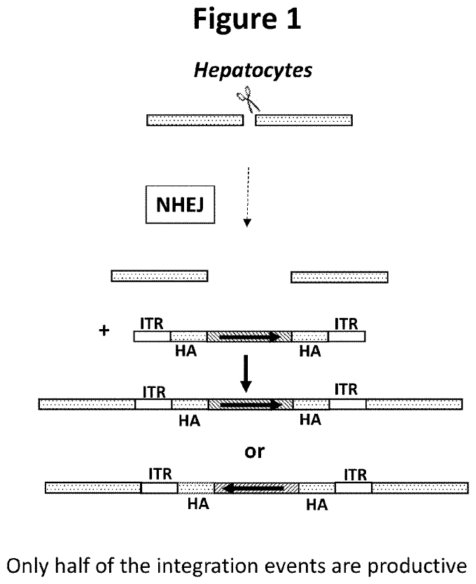

FIG. 2 shows schematics of exemplary push-pull donor constructs. Panel A shows a push-pull construct with two transgenes that are tail-to-tail in orientation. One or both of the transgenes may be codon diversified. ITR refers to inverted terminal repeat; poly A refers to a polyadenylation sequence; SA refers to a splice acceptor sequence. Panel B shows an exemplary specific push-pull iduronate-2-sulphatase (IDS) transgene construct, wherein one of the two IDS transgenes is codon diversified; ITR refers to inverted terminal repeat; bGH refers to the bovine Growth Hormone polyadenylation signal sequence (see Woychik et al. (1984) Proc Natl Acad Sci 81(13):3944-8); hGH refers to human Growth Hormone polyadenylation signal sequence; and F9SA refers to Factor 9 Splice Acceptor sequence.

FIG. 3 shows iduronate-2-sulfatase (IDS) activity in iPS-derived human hepatocytes following zinc finger nuclease mediated integration of 4 different AAV(AAV6) push pull IDS donor constructs (1, 2, 4, and 5). Panel A shows the results of hepatocytes transduced with low dose: 30 vg/cell of each AAV ZFN construct (left and right) and 240 vg/cell of AAV donor construct. Control refers to a donor construct containing a single IDS sequence. Panel B shows the results of hepatocytes transduced with high dose: 300 vg/cell of each AAV ZFN construct and 2400 vg/cell of AAV donor construct. Control refers to a donor construct containing a single IDS sequence. Panel C shows normalized IDS activity to the percentage of insertions and deletions (% indels) in cells transduced with low or high doses of AAV ZFN and AAV donor constructs. Control refers to a donor construct containing a single IDS sequence. Push pull donor constructs 2 and 4 exhibited 3-fold higher level of IDS production than the Control IDS donor construct (with single IDS sequence). Push pull donor constructs 1 and 5 exhibited 2-fold and 2.5-fold, respectively, higher level of IDS production than the Control IDS donor construct (with single IDS sequence). Mock refers to a sample which does not include ZFN/donor AAV treatment.

### DETAILED DESCRIPTION

The present disclosure provides compositions and methods for treating a disease (e.g., a genetic disorder (e.g., a lysosomal storage disease), an infectious disease, an acquired disorder, and a cancer) in a subject using a donor construct configured in a “push-pull” orientation to allow for improved expression of a therapeutic protein. More specifically, the present disclosure provides donor constructs which allow for improved expression of a therapeutic protein. These “push-pull” donor constructs are capable of integrating into a target genome with high precision and efficiency. The “push-pull” donor construct disclosed herein comprise a first nucleotide sequence encoding a first polypeptide and a second nucleotide sequence encoding a second polypeptide, wherein the first nucleotide sequence encoding a first polypeptide is oriented tail-to-tail to the second nucleotide sequence encoding a second polypeptide; and wherein the first nucleotide sequence and the second nucleotide sequence encode a polypeptide having the same amino acid sequence. The disclosure also provides vectors, cell and pharmaceutical compositions comprising such constructs.

The disclosure also provides methods of editing or modifying the genome of a cell by either integrating an exogenous sequence or by disrupting or deleting an undesired sequence using such donor construct. The methods disclosed herein include introducing into a cell in a subject such “push-pull” donor polynucleotide construct, which integrate with improved targeting and efficiency by means of nucleases (e.g., ZFN or TALEN).

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The term “herein” means the entire application.

Unless otherwise defined herein, scientific and technical terms used in this application shall have the meanings that are commonly understood by those of ordinary skill in the art to which this invention belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with one or more other embodiments of the invention, unless explicitly disclaimed or improper. Combination of embodiments are not limited to those specific combinations claimed via the multiple dependent claims.

All of the publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The term “chromosome,” as used herein, refers to a chromatin complex comprising all or a portion of the genome of a cell. The genome of a cell is often characterized by its karyotype, which is the collection of all the chromosomes that comprise the genome of the cell. The genome of a cell can comprise one or more chromosomes.

“Chromatin,” as used herein, refers to a nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) that extends between nucleosome cores. A molecule of histone H1 is generally associated with the linker DNA. For the purposes of the present disclosure, the term “chromatin” is meant to encompass all types of cellular nucleoprotein, both eukaryotic and prokaryotic. Cellular chromatin includes both chromosomal and episomal chromatin.

An “episome,” as used herein, refers to a replicating nucleic acid, nucleoprotein complex or other structure comprising a nucleic acid that is not part of the chromosomal karyotype of a cell. It is capable of existing and replicating either autonomously in a cell or as part of a host cell chromosome. Examples of episomes include plasmids and certain viral genomes.

The term “cleavage,” as used herein, refers to the breakage of the covalent backbone of a nucleic acid (e.g. DNA) molecule or polypeptide (e.g., protein) molecule. Cleavage can be initiated by a variety of methods including, but not limited to, enzymatic or chemical hydrolysis (e.g., hydrolysis of a phosphodiester bond in a nucleic acid molecule). With respect to nucleic acid molecules, both single-stranded cleavage and double-stranded cleavage are possible, and double-stranded cleavage can occur as a result of two distinct single-stranded cleavage events. Nucleic acid cleavage can result in the production of either blunt ends or staggered ends. In certain embodiments, fusion polypeptides are used for targeted double-stranded DNA cleavage. With respect to polypeptides, cleavage includes proteolytic cleavage which includes a breaking of the peptide bond between amino acids.

A “cleavage half-domain,” as used herein, refers to a polypeptide sequence which, in conjunction with a second polypeptide (either identical or different) forms a complex having cleavage activity (preferably double-strand cleavage activity). The terms “first and second cleavage half-domains;” “+ and—cleavage half-domains” and “right and left cleavage half-domains” are used interchangeably to refer to pairs of cleavage half-domains that dimerize.

An “engineered cleavage half-domain,” as used herein, refers to a cleavage half-domain that has been modified so as to form obligate heterodimers with another cleavage half-domain (e.g., another engineered cleavage half-domain). See, U.S. Pat. Nos. 7,888,121; 7,914,796; 8,034,598 and 8,823,618, incorporated herein by reference in their entireties.

The term “binding,” as used herein, refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}. “Non-specific binding” refers to, non-covalent interactions that occur between any molecule of interest (e.g. an engineered nuclease) and a macromolecule (e.g. DNA) that are not dependent on-target sequence.

A “binding protein,” as used herein, refers to a protein that is able to bind non-covalently to another molecule. A binding protein can bind to, for example, a DNA molecule (a DNA-binding protein), an RNA molecule (an RNA-binding protein) and/or a polypeptide or protein molecule (a protein-binding protein). In the case of a polypeptide- or protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “DNA binding molecule,” as used herein, refers to a molecule that can bind to DNA. Such DNA binding molecule can be a polypeptide, a domain of a protein, a domain within a larger protein or a polynucleotide. In some embodiments, the polynucleotide is DNA, while in other embodiments, the polynucleotide is RNA. In some embodiments, the DNA binding molecule is a protein domain of a nuclease (e.g. the zinc finger domain).

A “DNA binding protein” or “binding domain,” as used herein, refers to a protein, or a domain within a larger protein, that binds DNA in a sequence-specific manner, for example through one or more zinc fingers or through interaction with one or more Repeat Variable Diresidue (RVDs) in a zinc finger protein or TALE, respectively.

An “exogenous” molecule (e.g. nucleic acid sequence or protein) is a molecule that is not normally present in a cell, but can be introduced into a cell by one or more delivery methods. An exogenous molecule can comprise a therapeutic gene, a plasmid or episome introduced into a cell, a viral genome or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

As used herein, the term “product of an exogenous nucleic acid” includes both polynucleotide and polypeptide products, for example, transcription products (polynucleotides such as RNA) and translation products (polypeptides).

An “endogenous” molecule or sequence is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

“Eukaryotic” cells include, but are not limited to, fungal cells (such as yeast), plant cells, animal cells, mammalian cells and human cells (e.g., T-cells), including stem cells (pluripotent and multipotent).

A “fusion” molecule or any variation thereof is a molecule in which two or more subunit molecules are linked, preferably covalently. The subunit molecules can be the same chemical type of molecule or can be different chemical types of molecules. Examples of fusion molecules include, but are not limited to, fusion proteins (for example, a fusion between a zinc-finger DNA binding domain and a cleavage domain) and fusion nucleic acids (for example, a nucleic acid encoding the fusion protein). Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” as used herein, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression,” or “nucleotide expression” as used herein, refers to the conversion of the information contained in a gene or nucleotide sequence, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation.

A “region of interest,” as used herein, refers to any region of cellular chromatin, such as, for example, a gene or a non-coding sequence, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

The terms “codon diversified”, as used herein, refers to any nucleotide sequence in which the codon usage is altered as compared to the original undiversified sequence (e.g., the original designed or selected nuclease or wild-type or mutant donor). Codon diversified sequences may be obtained using any program, such as GeneGPS, and may result in sequences that recombine at a different rate than undiversified sequences and/or result in coding sequences that express higher levels of the encoded polypeptide as compared to undiversified sequence. DNA synthesis companies (such as ATUM and Blueheron) also have their internal algorithms for codon diversification.

A “TALE DNA binding domain” or “TALE” (Transcription activator-like effector), as used herein, refers to a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. Nos. 8,586,526 and 9,458,205. The term “TALEN” (Transcription activator-like effector nuclease) refers to one TALEN or a pair of TALENs (the members of the pair are referred to as “left and right” or “first and second” or “pair”) that dimerize to cleave the target gene. Zinc finger and TALE binding domains can be “engineered” to bind to a predetermined nucleotide sequence, for example, via engineering (altering one or more amino acids) of the recognition helix region of a naturally occurring zinc finger or TALE protein. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 8,568,526; 6,140,081; 6,453,242; and 6,534,261; see also International Patent Publication Nos. WO 98/53058; WO 98/53059; WO 98/53060; WO 02/016536; and WO 03/016496.

“Recombination,” as used herein, refers to a process of exchanging genetic information between two polynucleotides. For the purposes of this disclosure, “homologous recombination (HR)”, as used herein, refers to a specialized form of such exchange that takes place, for example, during repair of double-strand breaks in cells via homology-directed repair mechanisms. This process requires nucleotide sequence homology, and uses a “donor” molecule (i.e., exogenous DNA) as a template to repair a “target” molecule (i.e., a molecule with a double-stranded break), and is also referred to as “non-crossover gene conversion” or “short tract gene conversion,” because it leads to the transfer of genetic information from the donor to the target molecule. Without wishing to be bound by any particular theory, such transfer can involve mismatch correction of heteroduplex DNA that forms between the broken target and the donor, and/or “synthesis-dependent strand annealing,” in which the donor is used to re-synthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted nucleases as described herein create a double-stranded break in the target sequence (e.g., cellular chromatin) at a predetermined site, and a “donor” polynucleotide, having homology to the nucleotide sequence in the region of the break, can be introduced into the cell. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first target sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

The term “push-pull donor” construct refers to a polynucleotide comprising a first nucleotide sequence encoding a first polypeptide and a second nucleotide sequence encoding a second polypeptide, wherein the first nucleotide sequence encoding a first polypeptide is oriented tail-to-tail to the second nucleotide sequence encoding a second polypeptide, and wherein the first nucleotide sequence and the second nucleotide sequence encode a polypeptide having the same amino acid sequence

A tail to tail configuration refers to a configuration wherein the end of the first nucleotide sequence encoding a first polypeptide is located closer to the end (as opposed to the beginning) of the second nucleotide sequence encoding a second polypeptide.

The term “heterologous” means derived from a genotypically distinct entity from that of the rest of the entity to which it is being compared. For example, a polynucleotide introduced by genetic engineering techniques into a plasmid or vector derived from a different species is a heterologous polynucleotide.

The term “% Indel”, as used herein, refers to the percentage of insertions or deletions of several nucleotides in the target sequence of the genome.

“Modulation” (or variants thereof) of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP, TALE or CRISPR/Cas system as described herein. Thus, gene inactivation may be partial or complete.

The terms “operative linkage” and “operatively linked” (or “operably linked”) or variations thereof, as used herein, are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous. For example, a linker sequence can be located between both sequences. With respect to fusion polypeptides, the term “operatively linked” can refer to the fact that each of the components performs the same function in linkage to the other component as it would if it were not so linked. For example, with respect to a fusion polypeptide in which a ZFP or TALE DNA-binding domain is fused to an activation domain, the ZFP or TALE DNA-binding domain and the activation domain are in operative linkage if, in the fusion polypeptide, the ZFP or TALE DNA-binding domain portion is able to bind its target site and/or its binding site, while the activation domain is able to up-regulate gene expression. When a fusion polypeptide in which a ZFP or TALE DNA-binding domain is fused to a cleavage domain, the ZFP or TALE DNA-binding domain and the cleavage domain are in operative linkage if, in the fusion polypeptide, the ZFP or TALE DNA-binding domain portion is able to bind its target site and/or its binding site, while the cleavage domain is able to cleave DNA in the vicinity of the target site.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

A “functional” protein, polypeptide, polynucleotide or nucleic acid refers to any protein, polypeptide, polynucleotide or nucleic acid that provides the same function as the wild-type protein, polypeptide, polynucleotide or nucleic acid. A “functional fragment” of a protein, polypeptide, polynucleotide or nucleic acid is a protein, polypeptide, polynucleotide or nucleic acid whose sequence is not identical to the full-length protein, polypeptide or nucleic acid, yet retains the same function as the full-length protein, polypeptide, polynucleotide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and International Patent Publication No. WO 98/44350.

The term “safe-harbor locus or site,” as used herein, is a genomic locus where genes or other genetic elements can be safely inserted and expressed, because they are known to be tolerant to genetic modification without any undesired effects.

The term “sequence” refers to a nucleotide sequence of any length, which can be DNA or RNA; can be linear, circular or branched and can be either single-stranded or double-stranded. The term “sequence” also refers to an amino acid sequence of any length. The term “transgene” or “donor gene” refers to a nucleotide sequence that is inserted into a genome. A transgene can be of any length, for example between 2 and 100,000,000 nucleotides in length (or any integer value therebetween or thereabove), between about 100 and 100,000 nucleotides in length (or any integer therebetween), between about 2000 and 20,000 nucleotides in length (or any value therebetween) or between about 5 and 15 kb (or any value therebetween).

The term “specificity” (or variations thereof), as used herein, refers to the nuclease being able to bind the target sequence in a specific location with precision. The terms “specificity” and “precision” are used interchangeably.

The terms “subject” and “patient” are used interchangeably and refer to mammals including, but not limited to, human patients and non-human primates, as well as experimental animals such as rabbits, dogs, cats, rats, mice, and other animals. Accordingly, the term “subject” or “patient” as used herein means any mammalian patient or subject to which the polynucleotides and polypeptides of the invention can be administered.

A “disease associated gene or protein” is one that is defective in some manner in a genetic (e.g., monogenic) disorder, infectious disease, acquired disorder, cancer, and the like.

The term “target nucleotide sequence” or “target site,” as used herein, refers to a nucleotide sequence located in the genome of a cell which is specifically recognized by a zinc finger nucleotide binding domain of the zinc finger nuclease protein of the disclosure.

The terms “treating” and “treatment” or variations thereof, as used herein, refer to reduction in severity and/or frequency of symptoms, elimination of symptoms and/or underlying cause, prevention of the occurrence of symptoms and/or their underlying cause, delaying the occurrence of symptoms and/or their underlying cause, and improvement or remediation of damage. The treatment may help decrease the dose of one or more other medications required to treat the disease, and/or improve the quality of life.

An “effective dose” or “effective amount,” as used herein, refers to a dose and/or amount of the composition given to a subject as disclosed herein, that can help treat or prevent he occurrence of symptoms.

A polynucleotide “vector” or “construct” is capable of transferring gene sequences to target cells. Typically, “vector construct,” “expression vector,” “expression construct,” “expression cassette,” and “gene transfer vector,” mean any nucleic acid construct capable of directing the expression of a gene of interest and which can transfer gene sequences to target cells. Thus, the term includes cloning, and expression vehicles, as well as integrating vectors.

As used herein, the term “variant” refers to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 86%, about 87%, about 88% about 89%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 86%, about 87%, about 88% about 89%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The term “zinc-finger DNA binding protein” or “zinc-finger nucleotide binding domain,” as used herein, refers to a protein, or a domain within a larger protein, that binds DNA in a sequence-specific manner through one or more zinc fingers, which are regions of amino acid sequence within the binding domain whose structure is stabilized through coordination of one or more zinc ions. The term zinc finger DNA binding protein is abbreviated as zinc finger protein or ZFP.

The term “zinc-finger nuclease protein” or “zinc-finger nuclease”, as used herein, refers to a protein comprising a zinc-finger DNA binding domain (ZFP) directly or indirectly linked to a DNA cleavage domain (e.g., a Fok I DNA cleavage domain). The term zinc-finger nuclease protein is abbreviated as zinc finger nuclease or ZFN. The cleavage domain may be connected directly to the ZFP. Alternatively, the cleavage domain is connected to the ZFP by way of a linker. The linker region is a sequence which comprises about 1-150 amino acids. Alternatively, the linker region is a sequence which comprises about 6-50 nucleotides. The term includes one ZFN as well as a pair of ZFNs (the members of the pair are referred to as “left and right” or “first and second” or “pair”) that dimerize to cleave the target gene. A pair of ZFNs can be referred to as “left and right”, “first and second” or “pair” and can dimerize to cleave a target gene.

The term “zinc finger nuclease variant” as used herein, refers to a 2-in-1 zinc finger nuclease variant.

As used herein, “delaying” or “slowing” the progression of a disease refers to preventing, deferring, hindering, slowing, retarding, stabilizing, and/or postponing development of the disease. This delay can be of varying lengths of time, depending on the history of the disease and/or individual being treated.

A “symptom,” as used herein, refers to a phenomenon or feeling of departure from normal function, sensation, or structure that is experienced by a subject. For example, a subject with LSD may have symptoms including but not limited to decline in functional abilities, neurologic deterioration, joint stiffness, immobility leading to wheelchair dependency, and difficulty breathing leading to required use of a mechanical ventilator. These symptoms can lead to a shortened life span.

### Push-Pull Donor Constructs

The present disclosure provides donor constructs which allow for improved expression of a therapeutic protein. These push-pull donor constructs are capable of integrating into a target genome with high precision and efficiency.

Thus, in one aspect, disclosed herein is an push-pull donor polynucleotide construct comprising in 5′ to 3′ orientation: a) a first Inverted Terminal Repeat (ITR) nucleotide sequence; b) a first nucleotide sequence encoding a first polypeptide; c) a second nucleotide sequence encoding a second polypeptide; and d) a second ITR nucleotide sequence, wherein the first nucleotide sequence encoding a first polypeptide is oriented tail-to-tail to the second nucleotide sequence encoding a second polypeptide; and wherein the first nucleotide sequence and the second nucleotide sequence encode a polypeptide having the same amino acid sequence. When the push-pull donor polynucleotide construct integrates into a genomic locus, the polynucleotide can integrate in two orientations, but only one of the two nucleotides encoding a polypeptide is expressed (i.e., transcribed and/or translated). Thus, when the donor polynucleotide integrates in a first orientation, the first nucleotide sequence is expressed after being integrated into a genomic locus. When the donor polynucleotide integrates in a second orientation, the second nucleotide sequence is expressed after being integrated into a genomic locus.

In some embodiments, the first nucleotide sequence encoding the first polypeptide is codon diversified. In some embodiments, the first nucleotide sequence encoding the first polypeptide is not codon diversified. In some embodiments the second nucleotide sequence encoding the second polypeptide is codon diversified. In some embodiments the second nucleotide sequence encoding the second polypeptide is not codon diversified. In some embodiments, the first nucleotide sequence encoding the first polypeptide and the second nucleotide sequence encoding the second polypeptide are each independently codon diversified. In some embodiments, neither the first nucleotide sequence encoding the first polypeptide nor the second nucleotide sequence encoding the second polypeptide is codon diversified.

In some embodiments, the push-pull donor polynucleotide construct further comprises a a) a first splice acceptor sequence operatively linked to the first nucleotide sequence encoding the first polypeptide; b) a second splice acceptor sequence operatively linked to the second nucleotide sequence encoding the second polypeptide. The splice acceptor site can be a 3′site of an intron, an alternative 3′ splice site, a site within an exon, or a site within an intron.

In some embodiments, the first splice acceptor sequence is selected from a Factor 9 Splice Acceptor (F9SA), a CFTR Splice Acceptor, a COL5A2 Splice Acceptor, a NF1 Splice Acceptor, a MLH1 Splice Acceptor, and an Albumin (ALB) Splice Acceptor. In some embodiments, the first splice acceptor sequence is Factor 9 Splice Acceptor (F9SA). In some embodiments, the first splice acceptor sequence is a CFTR Splice Acceptor. In some embodiments, the first splice acceptor sequence is a COL5A2 Splice Acceptor. In some embodiments, the first splice acceptor sequence is a NF1 Splice Acceptor. In some embodiments, the first splice acceptor sequence is a MLH1 Splice Acceptor. In some embodiments, the first splice acceptor sequence is an Albumin (ALB) Splice Acceptor.

In some embodiments, the second splice acceptor sequence is selected from a Factor 9 Splice Acceptor (F9SA), a CFTR Splice acceptor, a COL5A2 Splice acceptor, a NF1 Splice Acceptor, a MLH1 Splice Acceptor, and an Albumin (ALB) Splice Acceptor. In some embodiments, the second splice acceptor sequence is a Factor 9 Splice Acceptor (F9SA). In some embodiments, the second splice acceptor sequence is a CFTR Splice Acceptor. In some embodiments, the second splice acceptor sequence is a COL5A2 Splice Acceptor. In some embodiments, the second splice acceptor sequence is a NF1 Splice Acceptor. In some embodiments, the second splice acceptor sequence is a MLH1 Splice Acceptor. In some embodiments, the second splice acceptor sequence is an Albumin (ALB) Splice Acceptor.

In some embodiments, the first splice acceptor and the second splice acceptor site are each independently a Factor 9 Splice Acceptor (F9SA).

In some embodiments the second splice acceptor sequence comprises a nucleotide sequence that is the reverse complement of the nucleotide sequence of the first splice acceptor sequence.

In some embodiments, the first splice acceptor sequence comprises the nucleotide sequence set forth in SEQ ID NO: 178. In some embodiments, the first splice acceptor sequence comprises the nucleotide sequence set forth in SEQ ID NO: 182. In some embodiments, the second splice acceptor sequence comprises the nucleotide sequence set forth in SEQ ID NO: 178. In some embodiments, the second splice acceptor sequence comprises the nucleotide sequence set forth in SEQ ID NO: 182.

In some embodiments, the push-pull donor polynucleotide construct further comprises a a) a first polyadenylation (polyA) signal sequence operatively linked to the nucleotide sequence encoding the first polypeptide; and a second polyadenylation (polyA) signal sequence operatively linked to the nucleotide sequence encoding the second polypeptide. In some embodiments, the first poly A signal sequence and the second poly A signal sequence are the same. In some embodiments, the first poly A signal sequence and the second poly A signal sequence are different.

Exemplary poly A sequences include, but are not limited to, human Growth Hormone (hGH) polyA signal, a bovine Growth Hormone (bGH) polyA signal, a SV40 polyA signal, and a rbGlob polyA signal. In some embodiments, the first polyA signal sequence is selected from a human Growth Hormone (hGH) polyA signal, a bovine Growth Hormone (bGH) polyA signal, a SV40 polyA signal, and a rbGlob polyA signal. In some embodiments, the first polyadenylation (polyA) signal sequence is a human Growth Hormone (hGH) polyA signal. In some embodiments, the first polyA signal sequence is a bovine Growth Hormone (bGH) polyA signal. In some embodiments, the first polyA signal sequence is a SV40 polyA signal. In some embodiments, the first polyA signal sequence is a rbGlob polyA signal.

In some embodiments, the second polyA signal sequence is selected from a human Growth Hormone (hGH) polyA signal, a bovine Growth Hormone (bGH) polyA signal, a SV40 polyA signal, and a rbGlob polyA signal. In some embodiments, the second polyadenylation (polyA) signal sequence is a human Growth Hormone (hGH) polyA signal. In some embodiments, the second polyA signal sequence is a bovine Growth Hormone (bGH) polyA signal. In some embodiments, the second polyA signal sequence is a SV40 polyA signal. In some embodiments, the second polyA signal sequence is a rbGlob polyA signal.

In some embodiments, the first (polyA) signal sequence is a human Growth Hormone (hGH) polyA signal and the second poly A signal sequence is a bovine Growth Hormone (bGH) polyA signal. In some embodiments, the first (polyA) signal sequence is a bovine Growth Hormone (bGH) polyA signal and the second poly A signal sequence is a human Growth Hormone (hGH) polyA signal. In some embodiments, the first (polyA) signal sequence is a human Growth Hormone (hGH) polyA signal and the second poly A signal sequence is a SV40 polyA signal. In some embodiments, the first (polyA) signal sequence is a SV40 polyA signal and the second poly A signal sequence is a human Growth Hormone (hGH) polyA signal. In some embodiments, the first (polyA) signal sequence is a human Growth Hormone (hGH) polyA signal and the second poly A signal sequence is rbGlob polyA signal. In some embodiments, the first (polyA) signal sequence is a rbGlob polyA signal and the second poly A signal sequence is a human Growth Hormone (hGH) polyA signal. In some embodiments, the first (polyA) signal sequence is a bovine Growth Hormone (bGH) polyA signal and the second poly A signal sequence is a SV40 polyA signal. In some embodiments, the first (polyA) signal sequence is a SV40 polyA signal and the second poly A signal sequence is a bovine Growth Hormone (bGH) polyA signal. In some embodiments, the first (polyA) signal sequence is a bovine Growth Hormone (bGH) polyA signal and the second poly A signal sequence is rbGlob polyA signal. In some embodiments, the first (polyA) signal sequence is a rbGlob polyA signal and the second poly A signal sequence is a bovine Growth Hormone (bGH) polyA signal. In some embodiments, the first (polyA) signal sequence is a SV40 polyA signal and the second poly A signal sequence is rbGlob polyA signal. In some embodiments, the first (polyA) signal sequence is a rbGlob polyA signal and the second poly A signal sequence is a SV40 polyA signal.

In some embodiments, the first polyA signal sequence comprises the nucleotide sequence set forth in SEQ ID NO: 179. In some embodiments, the first polyA signal sequence comprises the nucleotide sequence set forth in SEQ ID NO: 180. In some embodiments, the second polyA signal sequence comprises the nucleotide sequence set forth in SEQ ID NO: 179. In some embodiments, the second polyA signal sequence comprises the nucleotide sequence set forth in SEQ ID NO: 180. In some embodiments, the first polyA signal sequence comprises the nucleotide sequence set forth in SEQ ID NO: 179 and the second polyA signal sequence comprises the nucleotide sequence set forth in SEQ ID NO: 180. In some embodiments, the first polyA signal sequence comprises the nucleotide sequence set forth in SEQ ID NO: 180 and the second polyA signal sequence comprises the nucleotide sequence set forth in SEQ ID NO: 179.

In some embodiments, the push-pull donor polynucleotide construct comprises a first and a second inverted terminal repeat (ITR) sequences. ITR are comprised of a nucleotide sequence that is followed by its reverse complement. Examples of inverted repeats include direct repeats, tandem repeats and palindromes. The ITR may be 5′ITR, a 3′ITR or both. The ITRs play a role in the integration of the viral construct into the host genome and rescue the viral construct from the host genome.

In some embodiments, the first ITR sequence comprises the nucleotide sequence set forth in SEQ ID NO: 177. In some embodiments, the first ITR sequence comprises the nucleotide sequence set forth in SEQ ID NO: 181. In some embodiments, the second ITR sequence comprises the nucleotide sequence set forth in SEQ ID NO: 177. In some embodiments, the second ITR comprises the nucleotide sequence set forth in SEQ ID NO: 181.

In some embodiments, the push-pull donor polynucleotide construct of the disclosure comprises from 5′ to 3′ orientation: a) a 5′ITR; b) a first splice acceptor sequence; c) a first nucleotide sequence encoding a first polypeptide; d) a first polyadenylation (polyA) signal sequence; e) a second polyA signal sequence; f) a second nucleotide sequence encoding a second polypeptide; g) a second splice acceptor sequence; and h) a 3′ITR. The second polyA signal sequence, the second nucleotide sequence, and the second splice acceptor sequence are oriented in tail-to-tail to the first splice acceptor sequence, the first nucleotide sequence, and the first polyA signal sequence. When the push-pull donor polynucleotide construct integrates into a genomic locus, the polynucleotide can integrate in two orientations, but only one of the two nucleotides encoding a polypeptide is expressed (i.e., transcribed and/or translated). Thus, in one orientation, the first nucleotide sequence is expressed after being integrated into a genomic locus. In another orientation, the second nucleotide sequence is expressed after being integrated into a genomic locus.

In some embodiments, the first sequence encoding the first polypeptide or the second nucleotide sequence encoding the second polypeptide encodes a therapeutic polypeptide. In some embodiments, the therapeutic polypeptide includes but is not limited to, iduronate-2-sulphatase (IDS), alpha-L-iduronidase (IDUA), alpha-D-mannosidase, N-aspartyl-beta-glucosaminidase, lysosomal acid lipase, cystinosin, lysosomal associated membrane protein 2, alpha-galactosidase A, acid ceramidase, alpha fucosidase, cathepsin A, acid beta-glucocerebrosidase, beta galactosidase, beta hexosaminidase A, beta hexosaminidase B, beta hexosaminidase, GM2 ganglioside activator, GLcNAc-1-phosphotransferase, Beta-galactosylceramidase, arylsulfatase A, heparan N-sulfatase, alpha-N-acetylglucosaminidase, acetyl CoA:alpha-glucosaminide acetyltransferase, N-acetyl glucosamine-6-sulfatase, arylsulfatase B, beta-glucuronidase, hyaluronidase, neuraminidase, mucolipin-1, formylglycine-generating enzyme, palmitoyl-protein thioesterase 1, tripeptidyl peptidase 1, CLN3 protein, cysteine string protein alpha, CLN5 protein, CLN6 protein, CLN7 protein, CLN8 protein, acid sphingomyelinase, NPC 1, NPC 2, phenylalanine hydroxylase, acid alpha-glucosidase, cathepsin K, sialin, alpha-N-acetylgalactosaminidase, glucose-6-phosphatase, solute carrier family 37 member 4, argininosuccinate synthase 1, solute carrier family 25 member 13, and ornithine transcarbamylase (OTC).

In some embodiments, the first nucleotide sequence encoding the first polypeptide and/or the second nucleotide sequence encoding the second polypeptide includes but is not limited to MAN2B1, AGA, LIPA, CTNS, LAMP2, GLA, ASAH1, FUCA1, CTSA, GBA, GLB1, HEXB, HEXA, GM2A, GNPTAB, GALC, ARSA, IDUA, IDS, SGSH, NAGLU, GSNAT, GNS, GALNS, GLB1, ARSB, GUSB, HYAL1, NEU1, GNPTG, MCOLN1, SUMF1, PPT1, TPP1, CLN3, DNAJC5, CLN5, CLN6, CLN7, CLN8, SMPD1, SMPD1, NPC1, NPC2, PAH, GAA, CTSK, SLC17A5, NAGA, G6PC, SLC37A4, ASS1, SLC25A13 and OTC.

In some embodiments, the first nucleotide sequence encoding a first polypeptide comprises the nucleotide sequence set forth in SEQ ID NOs: 184-193. In some embodiments, the first nucleotide sequence encoding a first polypeptide comprises the nucleotide sequence set forth in SEQ ID NO: 184. In some embodiments, the first nucleotide sequence encoding a first polypeptide comprises the nucleotide sequence set forth in SEQ ID NO: 185. In some embodiments, the first nucleotide sequence encoding a first polypeptide comprises the nucleotide sequence set forth in SEQ ID NO: 186. In some embodiments, the first nucleotide sequence encoding a first polypeptide comprises the nucleotide sequence set forth in SEQ ID NO: 187. In some embodiments, the first nucleotide sequence encoding a first polypeptide comprises the nucleotide sequence set forth in SEQ ID NO: 188. In some embodiments, the first nucleotide sequence encoding a first polypeptide comprises the nucleotide sequence set forth in SEQ ID NO: 189. In some embodiments, the first nucleotide sequence encoding a first polypeptide comprises the nucleotide sequence set forth in SEQ ID NO: 190. In some embodiments, the first nucleotide sequence encoding a first polypeptide comprises the nucleotide sequence set forth in SEQ ID NO: 191. In some embodiments, the first nucleotide sequence encoding a first polypeptide comprises the nucleotide sequence set forth in SEQ ID NO: 192. In some embodiments, the first nucleotide sequence encoding a first polypeptide comprises the nucleotide sequence set forth in SEQ ID NO: 193.

In some embodiments, the second nucleotide sequence encoding a second polypeptide comprises the nucleotide sequence set forth in SEQ ID NOs: 184-193. In some embodiments, the second nucleotide sequence encoding a second polypeptide comprises the nucleotide sequence set forth in SEQ ID NO: 184. In some embodiments, the second nucleotide sequence encoding a second polypeptide comprises the nucleotide sequence set forth in SEQ ID NO: 185. In some embodiments, the second nucleotide sequence encoding a second polypeptide comprises the nucleotide sequence set forth in SEQ ID NO: 186. In some embodiments, the second nucleotide sequence encoding a second polypeptide comprises the nucleotide sequence set forth in SEQ ID NO: 187. In some embodiments, the second nucleotide sequence encoding a second polypeptide comprises the nucleotide sequence set forth in SEQ ID NO: 188. In some embodiments, the second nucleotide sequence encoding a second polypeptide comprises the nucleotide sequence set forth in SEQ ID NO: 189. In some embodiments, the second nucleotide sequence encoding a second polypeptide comprises the nucleotide sequence set forth in SEQ ID NO: 190. In some embodiments, the second nucleotide sequence encoding a second polypeptide comprises the nucleotide sequence set forth in SEQ ID NO: 191. In some embodiments, the second nucleotide sequence encoding a second polypeptide comprises the nucleotide sequence set forth in SEQ ID NO: 192. In some embodiments, the second nucleotide sequence encoding a second polypeptide comprises the nucleotide sequence set forth in SEQ ID NO: 193.

In some embodiments, the donor construct comprises the nucleotide sequence set forth in any one of SEQ ID NOs: 173-176. In some embodiments, the donor construct comprises the nucleotide sequence set forth in SEQ ID NO: 173. In some embodiments, the donor construct comprises the nucleotide sequence set forth in SEQ ID NO: 174. In some embodiments, the donor construct comprises the nucleotide sequence set forth in SEQ ID NO: 175. In some embodiments, the donor construct comprises the nucleotide sequence set forth in SEQ ID NO: 176.

In some embodiments, nucleotide sequence of the donor construct of the disclosure comprises at least about 60%, about 65%, about 70%, about 75%, about 80%, about 85%, about 90%, about 91%, about 92%, about 93%, about 94%, about 95%, about 96%, about 97%, about 98%, about 99% or more sequence identity to any of the sequences disclosed herein, as determined by sequence alignment programs known by skilled artisans. In some embodiments, the amino acid sequence of the donor construct of the disclosure comprises at least about 60%, about 65%, about 70%, about 75%, about 80%, about 85%, about 90%, about 91%, about 92%, about 93%, about 94%, about 95%, about 96%, about 97%, about 98%, about 99% or more sequence identity to any of the sequences disclosed herein, as determined by sequence alignment programs known by skilled artisans.

### Vectors and Delivery Systems

In one aspect, the present disclosure provides vectors comprising the push-pull donor polynucleotide constructs described herein. The push-pull donor polynucleotide constructs described herein may be delivered in vivo or ex vivo by any suitable vector system, including, but not limited to, plasmid vectors, a mini-circle and a linear DNA form, non-viral vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more of the sequences needed for treatment. Host cells containing said polynucleotide construct or vectors are also provided. Any of the foregoing push-pull donor polynucleotide construct, vectors or pharmaceutical compositions may be used in the methods disclosed herein.

Viral vector systems may also be used. Viral based systems for the delivery of the push-pull donor polynucleotide construct, transgenes, zinc finger proteins (ZFPs) and zinc finger nucleases (ZFNs) disclosed herein include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been measured in many different cell types and target tissues.

In some embodiments, adeno-associated virus (“AAV”) vectors are also used to transduce cells with push-pull donor constructs or zinc finger nuclease constructs as described herein. AAV serotypes that may be employed, including by non-limiting example, AAV1, AAV2, AAV3, AAV4, AAV5, AAV6, AAV8, AAV 8.2, AAV9 and AAV rh10 and pseudotyped AAV such as AAV2/8, AAV2/5 and AAV2/6 can also be used in accordance with the present invention. In some embodiments, the AAV is AAV1. In some embodiments, the AAV is AAV2. In some embodiments, the AAV is AAV3. In some embodiments, the AAV is AAV4. In some embodiments, the AAV is AAV5. In some embodiments, the AAV is AAV6. In some embodiments, the AAV is AAV8. In some embodiments, the AAV is AAV8.2. In some embodiments, the AAV is AAV9. In some embodiments, the AAV is AAVrh10. In some embodiments, the AAV is AAV2/5. In some embodiments, the AAV is AAV2/6.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Packaging cells are used to form virus particles (e.g., AAV particles) that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

Non-viral vector delivery systems include DNA plasmids, naked nucleic acid, mRNA, and nucleic acid complexed with a delivery vehicle such as a liposome or poloxamer. Methods of non-viral delivery of nucleic acids include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024.

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al. (2009) Nature Biotechnology 27(7):643).

Gene therapy vectors can be delivered in vivo by administration to an individual subject, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual subject (e.g., lymphocytes, bone marrow aspirates, tissue biopsy) or universal donor hematopoietic stem cells, followed by reimplantation of the cells into a subject, usually after selection for cells which have incorporated the vector.

Vectors (e.g., retroviruses, adenoviruses, liposomes, etc.) containing the donor or nuclease constructs disclosed herein can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

It will be apparent that the nuclease-encoding sequences and donor constructs can be delivered using the same or different systems. For example, a donor polynucleotide can be carried by a plasmid, while the one or more nucleases can be carried by an AAV vector. In certain embodiments, the nuclease and donors are both delivered using AAV vectors (e.g., both using AAV2, both using AAV6, both using AAV2/6, nuclease using AAV2, AAV6 or AAV2/6 and donor using AAV 2, AAV6 or AAV2/6). Furthermore, the different vectors can be administered by the same or different routes (intramuscular injection, intravenous injection, intraperitoneal administration and/or intramuscular injection. The vectors can be delivered simultaneously or in any sequential order.

### Pharmaceutical Composition

In one aspect, the disclosure relates to a pharmaceutical composition (also referred to as a “formulation” or an “article of manufacture” or a “drug product” or a “set of drug products”) comprising any of the nucleic acids, proteins or vectors described herein. In some embodiments, the pharmaceutical composition comprises a push-pull donor polynucleotide construct as disclosed herein. In some embodiments, the pharmaceutical composition comprises a push-pull donor polynucleotide construct as disclosed herein and further comprises a first polynucleotide encoding a first zinc finger nuclease (ZFN) and a second polynucleotide encoding a second zinc finger nuclease (ZFN) as disclosed herein. In some embodiments, the pharmaceutical composition comprises a push-pull donor polynucleotide construct as disclosed herein and further comprises a polynucleotide encoding one or more zinc finger nucleases as disclosed herein. In certain embodiments, the DNA binding domain of one or more of the nucleases used for in vivo cleavage and/or targeted cleavage of the genome of a cell comprises a zinc finger protein. In some embodiments, the zinc finger protein is non-naturally occurring in that it is engineered to bind to a target site of choice. Exemplary zinc finger proteins are described in e.g., Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 8,841,260; 8,772,453; 8,703,489; 8,409,861; 7,888,121; 7,361,635; 7,262,054; 7,253,273; 7,153,949; 7,070,934; 7,067,317; 7,030,215; 6,903,185; 6,794,136; 6,689,558; 6,599,692; 6,534,261; 6,503,717; 6,479,626; 6,453,242; 6,200,759; 6,140,081; 6,013,453; 6,007,988; 5,789,538; 5,925,523; and U.S. Patent Publication Nos. 20200246486, 2005/0064474; 2007/0218528; and 2005/0267061, all incorporated herein by reference in their entireties.

In some embodiments, the pharmaceutical composition comprises a polynucleotide encoding a 2-in-1 zinc finger nuclease.

In some embodiments, the pharmaceutical composition comprises a vector as described herein. In some embodiments, the pharmaceutical composition comprises a vector comprising a push-pull donor polynucleotide construct as described herein and further comprises a vector comprising a first polynucleotide encoding a first zinc finger nuclease and a vector comprising a second polynucleotide encoding a second zinc finger nuclease as disclosed herein. In some embodiments, the pharmaceutical composition comprises a vector comprising a vector comprising a push-pull donor polynucleotide construct as described herein and further comprises a vector comprising a polynucleotide encoding one or more zinc finger nucleases as disclosed herein. In some embodiments, the pharmaceutical composition comprises a vector comprising a vector comprising a push-pull donor polynucleotide construct as described herein and further comprises a vector comprising a polynucleotide encoding a 2-in-1 zinc finger nuclease as disclosed herein.

Pharmaceutical compositions for both ex vivo and in vivo administrations include suspensions in liquid or emulsified liquids. The active ingredients often are mixed with excipients which are pharmaceutically acceptable and compatible with the active ingredient. Suitable excipients include, for example, water, saline, dextrose, glycerol, ethanol or the like, and combinations thereof. In addition, the composition may contain minor amounts of auxiliary substances, such as, wetting or emulsifying agents, pH buffering agents, stabilizing agents or other reagents that enhance the effectiveness of the pharmaceutical composition.

Pharmaceutically acceptable carriers are determined in part by the particular composition being administered, as well as by the particular method used to administer the composition. Accordingly, there is a wide variety of suitable formulations of pharmaceutical compositions available (see, e.g., Remington's Pharmaceutical Sciences, 17th ed., 1989).

The ratio of the polynucleotide encoding the zinc finger nucleases to the push pull donor construct as disclosed herein, in the pharmaceutical composition varies from e.g., 1:0.1 to 1:40. The ratio of the polynucleotide encoding the zinc finger nucleases to the push pull donor construct as disclosed herein, in the pharmaceutical composition varies from e.g., 3:2 to 1:4. The ratio of the polynucleotide encoding the first zinc finger nuclease: the polynucleotide encoding the second zinc finger: the push-pull donor polynucleotide construct in the pharmaceutical composition varies from. e.g., 0.1:0.1:20 to 1:1:40. The ratio of the polynucleotide encoding the first zinc finger nuclease: the polynucleotide encoding the second zinc finger: the push-pull donor polynucleotide construct in the pharmaceutical composition varies from. e.g., 3:3:4 to 1:1:8. The ratio of the polynucleotide encoding the first zinc finger nuclease: the polynucleotide encoding the second zinc finger: the push-pull donor polynucleotide construct in the pharmaceutical composition includes but is not limited to, e.g., 1:1:8, 1:1:4, 1:1:2, and 3:3:4. In some embodiments, the ratio of the polynucleotide encoding the first zinc finger nuclease: the polynucleotide encoding the second zinc finger: the push-pull donor polynucleotide construct in the pharmaceutical composition is 1:1:8. In some embodiments, the ratio of the polynucleotide encoding the first zinc finger nuclease: the polynucleotide encoding the second zinc finger: the push-pull donor polynucleotide construct in the pharmaceutical composition is 1:1:4. In some embodiments, the ratio of the polynucleotide encoding the first zinc finger nuclease: the polynucleotide encoding the second zinc finger: the push-pull donor polynucleotide construct in the pharmaceutical composition is 1:1:2. In some embodiments, the ratio of the polynucleotide encoding the first zinc finger nuclease: the polynucleotide encoding the second zinc finger: the push-pull donor polynucleotide construct in the pharmaceutical composition is 3:3:4.

The ratio of the polynucleotide encoding the zinc finger nucleases to the push pull donor construct as disclosed herein, in the pharmaceutical composition varies from e.g., 1:0.1 to 1:40. In some embodiments, the ratio of the polynucleotide encoding the 2-in-1 zinc finger nuclease: the push-pull donor polynucleotide construct in the composition varies from 3:2 to 1:4. In some embodiments, the ratio of the polynucleotide encoding the 2-in-1 zinc finger nuclease: the push-pull donor polynucleotide construct in the composition includes but is not limited to, e.g., 1:4, 1:2, 1:1 and 3:2. In some embodiments, the ratio of the polynucleotide encoding the 2-in-1 zinc finger nuclease: the push-pull donor polynucleotide construct in the composition is 1:4. In some embodiments, the ratio of the polynucleotide encoding the 2-in-1 zinc finger nuclease: the push-pull donor polynucleotide construct in the pharmaceutical composition is 1:2. In some embodiments, the ratio of the polynucleotide encoding the 2-in-1 zinc finger nuclease: the push-pull donor polynucleotide construct in the pharmaceutical composition is 1:1. In some embodiments, the ratio of the polynucleotide encoding the 2-in-1 zinc finger nuclease: the push-pull donor polynucleotide construct in the pharmaceutical composition is 3:2.

The ratio of the vector comprising the polynucleotide encoding the zinc finger nucleases to the push pull donor construct as disclosed herein varies from e.g., 1:0.1 to 1:40. The ratio of the vector comprising the polynucleotide encoding the zinc finger nucleases to the vector comprising the push pull donor construct as disclosed herein, varies from, e.g., 3:2 to 1:4. The ratio of the vector comprising the polynucleotide encoding the first zinc finger nuclease: the polynucleotide encoding the second zinc finger: the push-pull donor polynucleotide varies from. e.g., 0.1:0.1:20 to 1:1:40. The ratio of the vector comprising the polynucleotide encoding the first zinc finger nuclease: the polynucleotide encoding the second zinc finger: the push-pull donor polynucleotide construct varies from. e.g., 3:3:4 to 1:1:8. The ratio of the vector comprising the polynucleotide encoding the first zinc finger nuclease: the polynucleotide encoding the second zinc finger: the push-pull donor polynucleotide construct includes but is not limited to, e.g., 1:1:8, 1:1:4, 1:1:2, and 3:3:4. In some embodiments, the ratio of the vector comprising the first polynucleotide encoding the first zinc finger nuclease: the vector comprising the second polynucleotide encoding the second zinc finger: the vector comprising the push-pull donor polynucleotide construct is 1:1:8. In some embodiments, the ratio of the vector comprising the first polynucleotide encoding the first zinc finger nuclease: the vector comprising the second polynucleotide encoding the second zinc finger: the vector comprising the push-pull donor polynucleotide construct is 1:1:4. In some embodiments, the ratio of the vector comprising the first polynucleotide encoding the first zinc finger nuclease: the vector comprising the second polynucleotide encoding the second zinc finger: the vector comprising the push-pull donor polynucleotide construct is 1:1:2. In some embodiments, the ratio of the vector comprising the first polynucleotide encoding the first zinc finger nuclease: the vector comprising the second polynucleotide encoding the second zinc finger: the vector comprising the push-pull donor polynucleotide construct is 3:3:4.

The ratio of the vector comprising the polynucleotide encoding the zinc finger nucleases to the push pull donor construct as disclosed herein, varies from e.g., 1:0.1 to 1:40. In some embodiments, the ratio of the vector comprising polynucleotide encoding the 2-in-1 zinc finger nuclease: the push-pull donor polynucleotide construct varies from 3:2 to 1:4. In some embodiments, the ratio of the vector comprising the polynucleotide encoding the 2-in-1 zinc finger nuclease: the push-pull donor polynucleotide construct includes but is not limited to, e.g., 1:4, 1:2, 1:1 and 3:2. In some embodiments, the ratio of the vector comprising the polynucleotide encoding the 2-in-1 zinc finger nuclease: the vector comprising the push-pull donor polynucleotide construct is 1:4. In some embodiments, the ratio of the vector comprising the polynucleotide encoding the 2-in-1 zinc finger nuclease: the vector comprising the push-pull donor polynucleotide construct is 1:2. In some embodiments, the ratio of the vector comprising the polynucleotide encoding the 2-in-1 zinc finger nuclease: the vector comprising the push-pull donor polynucleotide construct is 1:1. In some embodiments, the ratio of the vector comprising the polynucleotide encoding the 2-in-1 zinc finger nuclease: the vector comprising the push-pull donor polynucleotide construct is 3:2.

The pharmaceutical composition comprises a combination of the same or different composition in any concentrations. For example, provided herein is an article of manufacture comprising a set of drug products, which include two separate pharmaceutical compositions as follows: a first pharmaceutical composition comprising a purified AAV vector carrying both a first ZFN and a second ZFN pair and a second pharmaceutical composition comprising a purified AAV vector carrying a donor sequence comprising a transgene encoding a therapeutic protein for the treatment of a disease or disorder. One or both of pharmaceutical compositions may be individually formulated in phosphate buffered saline (PBS) containing CaCl₂), MgCl₂, NaCl, sucrose and a Poloxamer (e.g., Poloxamer P188) or in a Normal Saline (NS) formulation. In some embodiments, the composition comprises phosphate buffered saline (PBS) comprising approximately 1.15 mg/ML of sodium phosphate, 0.2 mg/mL potassium phosphate, 8.0 mg/mL sodium chloride, 0.2 mg/mL potassium chloride, 0.13 mg/mL calcium chloride, and 0.1 mg/mL Magnesium chloride. The PBS is further modified with 2.05 mg/mL sodium chloride, 10 mg/mL to 12 mg/mL of sucrose and 0.5 to 1.0 mg/mL of Kolliphor® (poloxamer or P188). Further, the article of manufacture may include any ratio of the two pharmaceutical compositions can be used.

2-in-1 Zinc Finger Nucleases

In some embodiments, the compositions and methods disclose herein comprise a nucleic acid encoding a 2-in-1 zinc finger nuclease variant. In some embodiments, the nucleic acid encoding a 2-in-1 zinc finger nuclease variant comprises: a) a polynucleotide encoding a first zinc finger nuclease; b) a polynucleotide encoding a second zinc finger nuclease; and c) a polynucleotide encoding a 2A self-cleaving peptide; wherein the polynucleotide encoding the 2A self-cleaving peptide is positioned between the polynucleotide encoding the first zinc finger nuclease and the polynucleotide encoding the second zinc finger nuclease. In some embodiments, the polynucleotide encoding the first zinc finger nuclease is codon diversified. In some embodiments, the polynucleotide encoding the first zinc finger nuclease is not codon diversified. In some embodiments the polynucleotide encoding the second zinc finger nuclease is codon diversified. In some embodiments the polynucleotide encoding the second zinc finger nuclease is not codon diversified. In some embodiments, the polynucleotide encoding the first zinc finger nuclease and the polynucleotide encoding the second zinc finger nuclease are each independently codon diversified. In some embodiments, neither the polynucleotide encoding the first zinc finger nuclease nor the polynucleotide encoding the second zinc finger nuclease is codon diversified.

In some embodiments, the nucleic acid encoding the 2-in-1 zinc finger nuclease variant further comprises a nucleic acid sequence selected from one or more of: a) one or more polynucleotide sequences encoding a nuclear localization sequence; b) a 5′ITR polynucleotide sequence; c) an enhancer polynucleotide sequence; d) a promoter polynucleotide sequence; e) a 5′UTR polynucleotide sequence; f) a chimeric intron polynucleotide sequence; g) one or more polynucleotide sequences encoding an epitope tag; h) one or more cleavage domains; i) a post-transcriptional regulatory element polynucleotide sequence; j) a polyadenylation signal sequence; k) a 3′UTR polynucleotide sequence; and 1) a 3′ITR polynucleotide sequence.

In some embodiments, the polynucleotide sequence encoding the first zinc finger nuclease comprises the nucleotide sequence of any one of SEQ ID NOs: 116-129. In some embodiments, the polynucleotide sequence encoding the first zinc finger nuclease comprises the nucleotide sequence of SEQ ID NO: 116. In some embodiments, the polynucleotide sequence encoding the first zinc finger nuclease comprises the nucleotide sequence of SEQ ID NO: 117. In some embodiments, the polynucleotide sequence encoding the first zinc finger nuclease comprises the nucleotide sequence of SEQ ID NO: 118. In some embodiments, the polynucleotide sequence encoding the first zinc finger nuclease comprises the nucleotide sequence of SEQ ID NO: 119. In some embodiments, the polynucleotide sequence encoding the first zinc finger nuclease comprises the nucleotide sequence of SEQ ID NO: 120. In some embodiments, the polynucleotide sequence encoding the first zinc finger nuclease comprises the nucleotide sequence of SEQ ID NO: 121. In some embodiments, the polynucleotide sequence encoding the first zinc finger nuclease comprises the nucleotide sequence of SEQ ID NO: 122. In some embodiments, the polynucleotide sequence encoding the first zinc finger nuclease comprises the nucleotide sequence of SEQ ID NO: 123. In some embodiments, the polynucleotide sequence encoding the first zinc finger nuclease comprises the nucleotide sequence of SEQ ID NO: 124. In some embodiments, the polynucleotide sequence encoding the first zinc finger nuclease comprises the nucleotide sequence of SEQ ID NO: 125. In some embodiments, the polynucleotide sequence encoding the first zinc finger nuclease comprises the nucleotide sequence of SEQ ID NO: 126. In some embodiments, the polynucleotide sequence encoding the first zinc finger nuclease comprises the nucleotide sequence of SEQ ID NO: 127. In some embodiments, the polynucleotide sequence encoding the first zinc finger nuclease comprises the nucleotide sequence of SEQ ID NO: 128. In some embodiments, the polynucleotide sequence encoding the first zinc finger nuclease comprises the nucleotide sequence of SEQ ID NO:129.

In some embodiments, the polynucleotide sequence encoding the first zinc finger nuclease comprises a nucleotide sequence encoding the amino acid sequence of any one of SEQ ID NOs: 136-137. In some embodiments, the polynucleotide sequence encoding the first zinc finger nuclease comprises a nucleotide sequence encoding the amino acid sequence of SEQ ID NOs: 136. In some embodiments, the polynucleotide sequence encoding the first zinc finger nuclease comprises a nucleotide sequence encoding the amino acid sequence of SEQ ID NOs: 137.

In some embodiments, the polynucleotide sequence encoding the second zinc finger nuclease comprises the nucleotide sequence of any one of SEQ ID NOs: 116-129. In some embodiments, the polynucleotide sequence encoding the second zinc finger nuclease comprises the nucleotide sequence of SEQ ID NO: 116. In some embodiments, the polynucleotide sequence encoding the second zinc finger nuclease comprises the nucleotide sequence of SEQ ID NO: 117. In some embodiments, the polynucleotide sequence encoding the second zinc finger nuclease comprises the nucleotide sequence of SEQ ID NO: 118. In some embodiments, the polynucleotide sequence encoding the second zinc finger nuclease comprises the nucleotide sequence of SEQ ID NO: 119. In some embodiments, the polynucleotide sequence encoding the second zinc finger nuclease comprises the nucleotide sequence of SEQ ID NO: 120. In some embodiments, the polynucleotide sequence encoding the second zinc finger nuclease comprises the nucleotide sequence of SEQ ID NO: 121. In some embodiments, the polynucleotide sequence encoding the second zinc finger nuclease comprises the nucleotide sequence of SEQ ID NO: 122. In some embodiments, the polynucleotide sequence encoding the second zinc finger nuclease comprises the nucleotide sequence of SEQ ID NO: 123. In some embodiments, the polynucleotide sequence encoding the second zinc finger nuclease comprises the nucleotide sequence of SEQ ID NO: 124. In some embodiments, the polynucleotide sequence encoding the second zinc finger nuclease comprises the nucleotide sequence of SEQ ID NO: 125. In some embodiments, the polynucleotide sequence encoding the second zinc finger nuclease comprises the nucleotide sequence of SEQ ID NO: 126. In some embodiments, the polynucleotide sequence encoding the second zinc finger nuclease comprises the nucleotide sequence of SEQ ID NO: 127. In some embodiments, the polynucleotide sequence encoding the second zinc finger nuclease comprises the nucleotide sequence of SEQ ID NO: 128. In some embodiments, the polynucleotide sequence encoding the second zinc finger nuclease comprises the nucleotide sequence of SEQ ID NO:129.

In some embodiments, the polynucleotide sequence encoding the second zinc finger nuclease comprises a nucleotide sequence encoding the amino acid sequence of any one of SEQ ID NOs: 136-137. In some embodiments, the polynucleotide sequence encoding the second zinc finger nuclease comprises a nucleotide sequence encoding the amino acid sequence of SEQ ID NOs: 136. In some embodiments, the polynucleotide sequence encoding the second zinc finger nuclease comprises a nucleotide sequence encoding the amino acid sequence of SEQ ID NOs: 137.

In some embodiments, the nucleic acid encoding the 2-in-1 zinc finger nuclease variant further comprises one or more polynucleotide sequences encoding one or more cleavage domains. Any suitable cleavage domain can be associated with (e.g., operatively linked) to a zinc finger DNA-binding domain (e.g., ZFP). In some embodiments, the two or more cleavage domains are the same. In some embodiments, the two or more cleavage domains have the same amino acid sequence. In some embodiments, the two or more cleavage domains have different amino acid sequences. In some embodiments, the two or more cleavage domains are encoded by a polynucleotide having the same nucleotide sequence. In some embodiments, the two or more cleavage domains are encoded by a polynucleotide having different nucleotide sequences. In some embodiments, the cleavage domain comprises a Fok I cleavage domain, which is active as a dimer. In some embodiments the polynucleotide sequence encoding the one or more Fok I cleavage domain is codon diversified. In some embodiments the polynucleotide sequence encoding the one or more Fok I cleavage domain is not codon diversified. In some embodiments the polynucleotide sequence encoding a first Fok I cleavage domain is operatively linked to the polynucleotide sequence encoding the first zinc finger DNA binding protein (ZFP). In some embodiments the polynucleotide sequence encoding a second Fok I cleavage domain is operatively linked to the polynucleotide sequence encoding the second zinc finger DNA binding protein (ZFP). In some embodiments the polynucleotide sequence encoding a first Fok I cleavage domain is located 3′ to the polynucleotide sequence encoding the first zinc finger DNA binding protein (ZFP). In some embodiments the polynucleotide sequence encoding a second Fok I cleavage domain is located 3′ to the polynucleotide sequence encoding the second zinc finger DNA binding protein (ZFP).

In some embodiments, the cleavage domain comprises one or more engineered cleavage half-domain (also referred to as dimerization domain mutants) that minimize or prevent homodimerization, as described, for example, in U.S. Pat. Nos. 8,772,453; 8,623,618; 8,409,861; 8,034,598; 7,914,796; and 7,888,121, the disclosures of all of which are incorporated by reference in their entireties herein. Amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains.

Exemplary engineered cleavage half-domains of Fok I that form obligate heterodimers include a pair in which a first cleavage half-domain includes mutations at amino acid residues at positions 490 and 538 of Fok I and a second cleavage half-domain includes mutations at amino acid residues 486 and 499.

Thus, in some embodiments, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. U.S. Pat. Nos. 7,914,796 and 8,034,598, the disclosures of which are incorporated by reference in their entireties. In some embodiments, the engineered cleavage half-domain comprises mutations at positions 486, 499 and 496 (numbered relative to wild-type Fok I), for instance mutations that replace the wild type Gln (Q) residue at position 486 with a Glu(E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In some embodiments, the engineered cleavage half-domain comprises mutations at positions 490, 538 and 537 (numbered relative to wild-type Fok I), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In some embodiments, the engineered cleavage half-domain comprises mutations at positions 490 and 537 (numbered relative to wild-type Fok I), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). See, e.g., U.S. Pat. No. 8,772,453. In some embodiments, the engineered cleavage half domain comprises the “Sharkey” and/or “Sharkey mutations” (see Guo et al. (2010) J. Mol. Biol. 400(1):96-107).

Engineered cleavage half-domains described herein can be prepared using any suitable method, for example, by site-directed mutagenesis of wild-type cleavage half-domains (Fok I) as described in U.S. Pat. Nos. 7,888,121; 7,914,796; 8,034,598; and 8,623,618 and U.S. Patent Publication Nos. 2019/0241877 and 2018/0087072.

In some embodiments, the polynucleotide sequence encoding the first zinc finger nuclease comprises the nucleotide sequence of any one of SEQ ID NOs: 71-84. In some embodiments, the polynucleotide sequence encoding the first zinc finger nuclease comprises the nucleotide sequence of SEQ ID NO: 71. In some embodiments, the polynucleotide sequence encoding the first zinc finger nuclease comprises the nucleotide sequence of SEQ ID NO: 72. In some embodiments, the polynucleotide sequence encoding the first zinc finger nuclease comprises the nucleotide sequence of SEQ ID NO: 73. In some embodiments, the polynucleotide sequence encoding the first zinc finger nuclease comprises the nucleotide sequence of SEQ ID NO: 74. In some embodiments, the polynucleotide sequence encoding the first zinc finger nuclease comprises the nucleotide sequence of SEQ ID NO: 75. In some embodiments, the polynucleotide sequence encoding the first zinc finger nuclease comprises the nucleotide sequence of SEQ ID NO: 76. In some embodiments, the polynucleotide sequence encoding the first zinc finger nuclease comprises the nucleotide sequence of SEQ ID NO: 77. In some embodiments, the polynucleotide sequence encoding the first zinc finger nuclease comprises the nucleotide sequence of SEQ ID NO: 78. In some embodiments, the polynucleotide sequence encoding the first zinc finger nuclease comprises the nucleotide sequence of SEQ ID NO: 79. In some embodiments, the polynucleotide sequence encoding the first zinc finger nuclease comprises the nucleotide sequence of SEQ ID NO: 80. In some embodiments, the polynucleotide sequence encoding the first zinc finger nuclease comprises the nucleotide sequence of SEQ ID NO: 81. In some embodiments, the polynucleotide sequence encoding the first zinc finger nuclease comprises the nucleotide sequence of SEQ ID NO: 82. In some embodiments, the polynucleotide sequence encoding the first zinc finger nuclease comprises the nucleotide sequence of SEQ ID NO: 83. In some embodiments, the polynucleotide sequence encoding the first zinc finger nuclease comprises the nucleotide sequence of SEQ ID NO:84.

In some embodiments, the polynucleotide sequence encoding the second zinc finger nuclease comprises the nucleotide sequence of any one of SEQ ID NOs: 71-84. In some embodiments, the polynucleotide sequence encoding the second zinc finger nuclease comprises the nucleotide sequence of SEQ ID NO: 71. In some embodiments, the polynucleotide sequence encoding the second zinc finger nuclease comprises the nucleotide sequence of SEQ ID NO: 72. In some embodiments, the polynucleotide sequence encoding the second zinc finger nuclease comprises the nucleotide sequence of SEQ ID NO: 73. In some embodiments, the polynucleotide sequence encoding the second zinc finger nuclease comprises the nucleotide sequence of SEQ ID NO: 74 In some embodiments, the polynucleotide sequence encoding the second zinc finger nuclease comprises the nucleotide sequence of SEQ ID NO: 75. In some embodiments, the polynucleotide sequence encoding the second zinc finger nuclease comprises the nucleotide sequence of SEQ ID NO: 76. In some embodiments, the polynucleotide sequence encoding the second zinc finger nuclease comprises the nucleotide sequence of SEQ ID NO: 77. In some embodiments, the polynucleotide sequence encoding the second zinc finger nuclease comprises the nucleotide sequence of SEQ ID NO: 78. In some embodiments, the polynucleotide sequence encoding the second zinc finger nuclease comprises the nucleotide sequence of SEQ ID NO: 79. In some embodiments, the polynucleotide sequence encoding the second zinc finger nuclease comprises the nucleotide sequence of SEQ ID NO: 80. In some embodiments, the polynucleotide sequence encoding the second zinc finger nuclease comprises the nucleotide sequence of SEQ ID NO: 81. In some embodiments, the polynucleotide sequence encoding the second zinc finger nuclease comprises the nucleotide sequence of SEQ ID NO: 82. In some embodiments, the polynucleotide sequence encoding the second zinc finger nuclease comprises the nucleotide sequence of SEQ ID NO: 83. In some embodiments, the polynucleotide sequence encoding the second zinc finger nuclease comprises the nucleotide sequence of SEQ ID NO:84.

In some embodiments, the polynucleotide sequence encoding the first zinc finger nuclease comprises a nucleotide sequence encoding the amino acid sequence of any one of SEQ ID NOs: 130-131. In some embodiments, the polynucleotide sequence encoding the first zinc finger nuclease comprises a nucleotide sequence encoding the amino acid sequence of SEQ ID NOs: 130. In some embodiments, the polynucleotide sequence encoding the first zinc finger nuclease comprises a nucleotide sequence encoding the amino acid sequence of SEQ ID NOs: 131.

In some embodiments, the polynucleotide sequence encoding the second zinc finger nuclease comprises a nucleotide sequence encoding the amino acid sequence of any one of SEQ ID NOs: 130-131. In some embodiments, the polynucleotide sequence encoding the second zinc finger nuclease comprises a nucleotide sequence encoding the amino acid sequence of SEQ ID NOs: 130. In some embodiments, the polynucleotide sequence encoding the second zinc finger nuclease comprises a nucleotide sequence encoding the amino acid sequence of SEQ ID NOs: 131.

In some embodiments, the nucleic acid encoding the 2-in-1 zinc finger nuclease variants further comprises one or more nucleotide sequences encoding one or more nuclear localization sequence (NLS). In some embodiments, the nucleic acid encoding the 2-in-1 zinc finger nuclease variant comprises a nucleotide sequence encoding a first nuclear localization sequence (NLS) and a nucleotide sequence encoding a second nuclear localization sequence (NLS), wherein the nucleotide sequence encoding first nuclear localization sequence (NLS) is located 5′ to the nucleotide sequence encoding the first zinc finger DNA binding protein (ZFP) and the nucleotide sequence encoding the second nuclear localization sequence (NLS) is located 5′ to the nucleotide sequence encoding the second zinc finger DNA binding protein (ZFP). In some embodiments, the nucleotide sequence encoding the first NLS is operatively linked to the nucleotide sequence encoding the first ZFP and the nucleotide sequence encoding the second NLS is operatively linked to the nucleotide sequence encoding the second ZFP. In some embodiments, the nucleotide sequence encoding the first NLS is codon diversified. In some embodiments, the nucleotide sequence encoding the first NLS is not codon diversified. In some embodiments, the nucleotide sequence encoding the second NLS is codon diversified. In some embodiments, the nucleotide sequence encoding the second NLS is not codon diversified. In some embodiments, the nucleotide sequence encoding each of the two or more NLS is the same. In some embodiments, the nucleotide sequence encoding each of the two or more NLS is the different. In some embodiments, each of the two or more NLS have the same amino acid sequence. In some embodiments, each of the two or more NLS have different amino acid sequences. In some embodiments, the polynucleotide encoding the first NLS comprises the nucleotide sequence set forth in any one of SEQ ID NO: 59-70 or 155. In some embodiments, the polynucleotide encoding the first NLS comprises the nucleotide sequence set forth in SEQ ID NO: 59. In some embodiments, the polynucleotide encoding the first NLS comprises the nucleotide sequence set forth in SEQ ID NO: 60. In some embodiments, the polynucleotide encoding the first NLS comprises the nucleotide sequence set forth in SEQ ID NO: 61. In some embodiments, the polynucleotide encoding the first NLS comprises the nucleotide sequence set forth in SEQ ID NO: 62. In some embodiments, the polynucleotide encoding the first NLS comprises the nucleotide sequence set forth in SEQ ID NO: 63. In some embodiments, the polynucleotide encoding the first NLS comprises the nucleotide sequence set forth in SEQ ID NO: 64. In some embodiments, the polynucleotide encoding the first NLS comprises the nucleotide sequence set forth in SEQ ID NO: 65. In some embodiments, the polynucleotide encoding the first NLS comprises the nucleotide sequence set forth in SEQ ID NO: 66. In some embodiments, the polynucleotide encoding the first NLS comprises the nucleotide sequence set forth in SEQ ID NO: 67. In some embodiments, the polynucleotide encoding the first NLS comprises the nucleotide sequence set forth in SEQ ID NO: 68. In some embodiments, the polynucleotide encoding the first NLS comprises the nucleotide sequence set forth in SEQ ID NO: 69. In some embodiments, the polynucleotide encoding the first NLS comprises the nucleotide sequence set forth in SEQ ID NO: 70. In some embodiments, the polynucleotide encoding the first NLS comprises the nucleotide sequence set forth in SEQ ID NO: 155. In some embodiments, the polynucleotide encoding the second NLS comprises the nucleotide sequence set forth in any one of SEQ ID NO: 59-70 or 155. In some embodiments, the polynucleotide encoding the second NLS comprises the nucleotide sequence set forth in SEQ ID NO: 59. In some embodiments, the polynucleotide encoding the second NLS comprises the nucleotide sequence set forth in SEQ ID NO: 60. In some embodiments, the polynucleotide encoding the second NLS comprises the nucleotide sequence set forth in SEQ ID NO: 61. In some embodiments, the polynucleotide encoding the second NLS comprises the nucleotide sequence set forth in SEQ ID NO: 62. In some embodiments, the polynucleotide encoding the second NLS comprises the nucleotide sequence set forth in SEQ ID NO: 63. In some embodiments, the polynucleotide encoding the second NLS comprises the nucleotide sequence set forth in SEQ ID NO: 64. In some embodiments, the polynucleotide encoding the second NLS comprises the nucleotide sequence set forth in SEQ ID NO: 65. In some embodiments, the polynucleotide encoding the second NLS comprises the nucleotide sequence set forth in SEQ ID NO: 66. In some embodiments, the polynucleotide encoding the second NLS comprises the nucleotide sequence set forth in SEQ ID NO: 67. In some embodiments, the polynucleotide encoding the second NLS comprises the nucleotide sequence set forth in SEQ ID NO: 68. In some embodiments, the polynucleotide encoding the second NLS comprises the nucleotide sequence set forth in SEQ ID NO: 69. In some embodiments, the polynucleotide encoding the second NLS comprises the nucleotide sequence set forth in SEQ ID NO: 70. In some embodiments, the polynucleotide encoding the first NLS comprises the nucleotide sequence set forth in SEQ ID NO: 155.

In some embodiments, the polynucleotide encoding the first NLS comprises a nucleotide sequence encoding the amino acid sequence set forth in any one of SEQ ID NO: 3-9 and 156. In some embodiments, the polynucleotide encoding the first NLS comprises a nucleotide sequence encoding the amino acid sequence set forth in SEQ ID NO: 3. In some embodiments, the polynucleotide encoding the first NLS comprises a nucleotide sequence encoding the amino acid sequence set forth in SEQ ID NO: 4. In some embodiments, the polynucleotide encoding the first NLS comprises a nucleotide sequence encoding the amino acid sequence set forth in SEQ ID NO:5. In some embodiments, the polynucleotide encoding the first NLS comprises a nucleotide sequence encoding the amino acid sequence set forth in SEQ ID NO: 6. In some embodiments, the polynucleotide encoding the first NLS comprises a nucleotide sequence encoding the amino acid sequence set forth in SEQ ID NO: 7. In some embodiments, the polynucleotide encoding the first NLS comprises a nucleotide sequence encoding the amino acid sequence set forth in SEQ ID NO: 8. In some embodiments, the polynucleotide encoding the first NLS comprises a nucleotide sequence encoding the amino acid sequence set forth in SEQ ID NO: 9. In some embodiments, the polynucleotide encoding the first NLS comprises a nucleotide sequence encoding the amino acid sequence set forth in SEQ ID NO: 156. In some embodiments, the polynucleotide encoding the second NLS comprises a nucleotide sequence encoding the amino acid sequence set forth in any one of SEQ ID NO: 3-9 and 156. In some embodiments, the polynucleotide encoding the second NLS comprises a nucleotide sequence encoding the amino acid sequence set forth in SEQ ID NO: 3. In some embodiments, the polynucleotide encoding the second NLS comprises a nucleotide sequence encoding the amino acid sequence set forth in SEQ ID NO: 4. In some embodiments, the polynucleotide encoding the second NLS comprises a nucleotide sequence encoding the amino acid sequence set forth in SEQ ID NO:5. In some embodiments, the polynucleotide encoding the second NLS comprises a nucleotide sequence encoding the amino acid sequence set forth in SEQ ID NO: 6. In some embodiments, the polynucleotide encoding the second NLS comprises a nucleotide sequence encoding the amino acid sequence set forth in SEQ ID NO: 7. In some embodiments, the polynucleotide encoding the second NLS comprises a nucleotide sequence encoding the amino acid sequence set forth in SEQ ID NO: 8. In some embodiments, the polynucleotide encoding the second NLS comprises a nucleotide sequence encoding the amino acid sequence set forth in SEQ ID NO: 9. In some embodiments, the polynucleotide encoding the second NLS comprises a nucleotide sequence encoding the amino acid sequence set forth in SEQ ID NO: 156.

In some embodiments, the polynucleotide sequence encoding the first zinc finger nuclease comprises the nucleotide sequence of any one of SEQ ID NOs: 139-152. In some embodiments, the polynucleotide sequence encoding the first zinc finger nuclease comprises the nucleotide sequence of SEQ ID NO: 139. In some embodiments, the polynucleotide sequence encoding the first zinc finger nuclease comprises the nucleotide sequence of SEQ ID NO: 140. In some embodiments, the polynucleotide sequence encoding the first zinc finger nuclease comprises the nucleotide sequence of SEQ ID NO: 141. In some embodiments, the polynucleotide sequence encoding the first zinc finger nuclease comprises the nucleotide sequence of SEQ ID NO: 142. In some embodiments, the polynucleotide sequence encoding the first zinc finger nuclease comprises the nucleotide sequence of SEQ ID NO: 143. In some embodiments, the polynucleotide sequence encoding the first zinc finger nuclease comprises the nucleotide sequence of SEQ ID NO: 144. In some embodiments, the polynucleotide sequence encoding the first zinc finger nuclease comprises the nucleotide sequence of SEQ ID NO: 145. In some embodiments, the polynucleotide sequence encoding the first zinc finger nuclease comprises the nucleotide sequence of SEQ ID NO: 146. In some embodiments, the polynucleotide sequence encoding the first zinc finger nuclease comprises the nucleotide sequence of SEQ ID NO: 147. In some embodiments, the polynucleotide sequence encoding the first zinc finger nuclease comprises the nucleotide sequence of SEQ ID NO: 148. In some embodiments, the polynucleotide sequence encoding the first zinc finger nuclease comprises the nucleotide sequence of SEQ ID NO: 149. In some embodiments, the polynucleotide sequence encoding the first zinc finger nuclease comprises the nucleotide sequence of SEQ ID NO: 150. In some embodiments, the polynucleotide sequence encoding the first zinc finger nuclease comprises the nucleotide sequence of SEQ ID NO: 151. In some embodiments, the polynucleotide sequence encoding the first zinc finger nuclease comprises the nucleotide sequence of SEQ ID NO: 152.

In some embodiments, the polynucleotide sequence encoding the second zinc finger nuclease comprises the nucleotide sequence of any one of SEQ ID NOs: 139-152. In some embodiments, the polynucleotide sequence encoding the second zinc finger nuclease comprises the nucleotide sequence of SEQ ID NO: 139. In some embodiments, the polynucleotide sequence encoding the second zinc finger nuclease comprises the nucleotide sequence of SEQ ID NO: 140. In some embodiments, the polynucleotide sequence encoding the second zinc finger nuclease comprises the nucleotide sequence of SEQ ID NO: 141. In some embodiments, the polynucleotide sequence encoding the second zinc finger nuclease comprises the nucleotide sequence of SEQ ID NO: 142. In some embodiments, the polynucleotide sequence encoding the second zinc finger nuclease comprises the nucleotide sequence of SEQ ID NO: 143. In some embodiments, the polynucleotide sequence encoding the second zinc finger nuclease comprises the nucleotide sequence of SEQ ID NO: 144. In some embodiments, the polynucleotide sequence encoding the second zinc finger nuclease comprises the nucleotide sequence of SEQ ID NO: 145. In some embodiments, the polynucleotide sequence encoding the second zinc finger nuclease comprises the nucleotide sequence of SEQ ID NO: 146. In some embodiments, the polynucleotide sequence encoding the second zinc finger nuclease comprises the nucleotide sequence of SEQ ID NO: 147. In some embodiments, the polynucleotide sequence encoding the second zinc finger nuclease comprises the nucleotide sequence of SEQ ID NO: 148. In some embodiments, the polynucleotide sequence encoding the second zinc finger nuclease comprises the nucleotide sequence of SEQ ID NO: 149. In some embodiments, the polynucleotide sequence encoding the second zinc finger nuclease comprises the nucleotide sequence of SEQ ID NO: 150. In some embodiments, the polynucleotide sequence encoding the second zinc finger nuclease comprises the nucleotide sequence of SEQ ID NO: 151. In some embodiments, the polynucleotide sequence encoding the second zinc finger nuclease comprises the nucleotide sequence of SEQ ID NO: 152.

In some embodiments, the nucleic acid encoding the 2-in-1 zinc finger nuclease variant further comprises one or more nucleotide sequences encoding one or more epitope tag. Epitope tags or expression tags refer to a peptide sequence engineered to be positioned 5′ or 3′ to a translated protein. Epitope tags include, for example one or more copies of FLAG, HA, CBP, GST, HBH, MBP, Myc, His, polyHis, S-tag, SUMO, TAP, TAGP, TRX, V5, GFP, RFP, YFP, and the like. “Expression tags” include sequences that encode reporters that may be operably linked to a desired gene sequence in order to monitor expression of the gene of interest.

In some embodiments, the nucleic acid encoding the 2-in-1 zinc finger nuclease variant further comprises one or more nucleotide sequences encoding one or more copies of an epitope tag. In some embodiments, the nucleic acid encoding the 2-in-1 zinc finger nuclease variant further comprise a first nucleotide sequence encoding a first epitope tag and a second nucleotide sequence encoding a second epitope tag. In some embodiments, each of said first epitope tag and second epitope tag is the same. In some embodiments, the first nucleotide sequence encoding the first epitope tag is located 5′ to the nucleotide sequence encoding the first ZFP, and the second nucleotide sequence encoding the second epitope tag is located 5′ to the nucleotide sequence encoding the second ZFP. In some embodiments, the first nucleotide sequence encoding the first epitope tag is located 5′ to the nucleotide sequence encoding the first NLS, and the second nucleotide sequence encoding the second epitope tag is located 5′ to the nucleotide sequence encoding the second NLS. In some embodiments, the first nucleotide sequence encoding the first epitope tag is located 3′ to the nucleotide sequence encoding the first ZFP, and the second nucleotide sequence encoding the second epitope tag is located 3′ to the nucleotide sequence encoding the second ZFP. In some embodiments, the first nucleotide sequence encoding the first epitope tag is located 3′ to the nucleotide sequence encoding the first NLS, and the second nucleotide sequence encoding the second epitope tag is located 3′ to the nucleotide sequence encoding the second NLS. In some embodiments, the first nucleotide sequence encoding the first epitope tag is codon diversified. In some embodiments, the first nucleotide sequence encoding the first epitope tag is not codon diversified. In some embodiments, the second nucleotide sequence encoding the second epitope tag is codon diversified. In some embodiments, the second nucleotide sequence encoding the second epitope tag is not codon diversified. In some embodiments, each of the two or more epitope tags has the same amino acid sequence. In some embodiments, each of the two or more epitope tags has different amino acid sequences. In some embodiments, each of the two or more epitope tags is encoded by a polynucleotide having the same nucleotide sequence. In some embodiments, each of the two or more epitope tags is encoded by a polynucleotide having different nucleotide sequences.

In some embodiments, the nucleic acid encoding the 2-in-1 zinc finger nuclease variant further comprises one or more nucleotide sequences encoding one or more copies of a FLAG tag. In some embodiments, the epitope tag is 3×FLAG. In some embodiments, the nucleic acid encoding the 2-in-1 zinc finger nuclease variant further comprise a first nucleotide sequence encoding a first FLAG tag and a second nucleotide sequence encoding a second FLAG tag. In some embodiments, each of said first FLAG tag and second FLAG tag is 3×FLAG. In some embodiments, the first nucleotide sequence encoding the first FLAG tag is located 5′ to the nucleotide sequence encoding the first ZFP, and the second nucleotide sequence encoding the second FLAG tag is located 5′ to the nucleotide sequence encoding the second ZFP. In some embodiments, the first nucleotide sequence encoding the first FLAG tag is located 5′ to the nucleotide sequence encoding the first NLS, and the second nucleotide sequence encoding the second FLAG tag is located 5′ to the nucleotide sequence encoding the second NLS. In some embodiments, the first nucleotide sequence encoding the first FLAG tag is located 3′ to the nucleotide sequence encoding the first ZFP, and the second nucleotide sequence encoding the second FLAG tag is located 3′ to the nucleotide sequence encoding the second ZFP. In some embodiments, the first nucleotide sequence encoding the first FLAG tag is located 3′ to the nucleotide sequence encoding the first NLS, and the second nucleotide sequence encoding the second FLAG tag is located 3′ to the nucleotide sequence encoding the second NLS. In some embodiments, the first nucleotide sequence encoding the first FLAG tag is codon diversified. In some embodiments, the first nucleotide sequence encoding the first FLAG tag is not codon diversified. In some embodiments, the second nucleotide sequence encoding the second FLAG tag is codon diversified. In some embodiments, the second nucleotide sequence encoding the second FLAG tag is not codon diversified. In some embodiments, each of the two or more FLAG tags has the same amino acid sequence. In some embodiments, each of the two or more FLAG tags has different amino acid sequences. In some embodiments, each of the two or more FLAG tags is encoded by a polynucleotide having the same nucleotide sequence. In some embodiments, each of the two or more FLAG tags is encoded by a polynucleotide having different nucleotide sequences.

In some embodiments, the nucleotide sequence encoding the first FLAG tag comprises the nucleotide sequence set forth in any one of SEQ ID NO: 15-16 or 50-58. In some embodiments, the nucleotide sequence encoding the first FLAG tag comprises the nucleotide sequence set forth in SEQ ID NO: 15. In some embodiments, the nucleotide sequence encoding the first FLAG tag comprises the nucleotide sequence set forth in SEQ ID NO: 16. In some embodiments, the nucleotide sequence encoding the first FLAG tag comprises the nucleotide sequence set forth in SEQ ID NO: 50. In some embodiments, the nucleotide sequence encoding the first FLAG tag comprises the nucleotide sequence set forth in SEQ ID NO: 51. In some embodiments, the nucleotide sequence encoding the first FLAG tag comprises the nucleotide sequence set forth in SEQ ID NO: 52. In some embodiments, the nucleotide sequence encoding the first FLAG tag comprises the nucleotide sequence set forth in SEQ ID NO: 53. In some embodiments, the nucleotide sequence encoding the first FLAG tag comprises the nucleotide sequence set forth in SEQ ID NO: 54. In some embodiments, the nucleotide sequence encoding the first FLAG tag comprises the nucleotide sequence set forth in SEQ ID NO: 55. In some embodiments, the nucleotide sequence encoding the first FLAG tag comprises the nucleotide sequence set forth in SEQ ID NO: 56. In some embodiments, the nucleotide sequence encoding the first FLAG tag comprises the nucleotide sequence set forth in SEQ ID NO: 57. In some embodiments, the nucleotide sequence encoding the first FLAG tag comprises the nucleotide sequence set forth in SEQ ID NO: 58. In some embodiments, the nucleotide sequence encoding the second FLAG tag comprises the nucleotide sequence set forth in any one of SEQ ID NO: 15-16 or 50-58. In some embodiments, the nucleotide sequence encoding the second FLAG tag comprises the nucleotide sequence set forth in SEQ ID NO: 15. In some embodiments, the nucleotide sequence encoding the second FLAG tag comprises the nucleotide sequence set forth in SEQ ID NO: 16. In some embodiments, the nucleotide sequence encoding the second FLAG tag comprises the nucleotide sequence set forth in SEQ ID NO: 50. In some embodiments, the nucleotide sequence encoding the second FLAG tag comprises the nucleotide sequence set forth in SEQ ID NO: 51. In some embodiments, the nucleotide sequence encoding the second FLAG tag comprises the nucleotide sequence set forth in SEQ ID NO: 52. In some embodiments, the nucleotide sequence encoding the second FLAG tag comprises the nucleotide sequence set forth in SEQ ID NO: 53. In some embodiments, the nucleotide sequence encoding the second FLAG tag comprises the nucleotide sequence set forth in SEQ ID NO: 54. In some embodiments, the nucleotide sequence encoding the second FLAG tag comprises the nucleotide sequence set forth in SEQ ID NO: 55. In some embodiments, the nucleotide sequence encoding the second FLAG tag comprises the nucleotide sequence set forth in SEQ ID NO: 56. In some embodiments, the nucleotide sequence encoding the second FLAG tag comprises the nucleotide sequence set forth in SEQ ID NO: 57. In some embodiments, the nucleotide sequence encoding the second FLAG tag comprises the nucleotide sequence set forth in SEQ ID NO: 58. In some embodiments, the nucleotide sequence encoding the first FLAG tag comprises a nucleotide sequence encoding the amino acid sequence set forth in any one of SEQ ID NO: 1-2. In some embodiments, the nucleotide sequence encoding the first FLAG tag comprises an nucleotide sequence encoding the amino acid sequence set forth in SEQ ID NO: 1. In some embodiments, the nucleotide sequence encoding the first FLAG tag comprises a nucleotide sequence encoding the amino acid sequence set forth in SEQ ID NO: 2. In some embodiments, the nucleotide sequence encoding the second FLAG tag comprises the nucleotide sequence encoding the amino acid sequence set forth in any one of SEQ ID NO: 1-2. In some embodiments, the nucleotide sequence encoding the second FLAG tag comprises a nucleotide sequence encoding the amino acid sequence set forth in SEQ ID NO: 1. In some embodiments, the nucleotide sequence encoding the second FLAG tag comprises a nucleotide sequence encoding amino acid sequence set forth in SEQ ID NO: 2.

In some embodiments, the polynucleotide sequence encoding the first zinc finger nuclease comprises the nucleotide sequence of any one of SEQ ID NOs: 17-23 and 25-31. In some embodiments, the polynucleotide sequence encoding the first zinc finger nuclease comprises the nucleotide sequence of SEQ ID NO: 17. In some embodiments, the polynucleotide sequence encoding the first zinc finger nuclease comprises the nucleotide sequence of SEQ ID NO: 18. In some embodiments, the polynucleotide sequence encoding the first zinc finger nuclease comprises the nucleotide sequence of SEQ ID NO: 19. In some embodiments, the polynucleotide sequence encoding the first zinc finger nuclease comprises the nucleotide sequence of SEQ ID NO: 20. In some embodiments, the polynucleotide sequence encoding the first zinc finger nuclease comprises the nucleotide sequence of SEQ ID NO: 21. In some embodiments, the polynucleotide sequence encoding the first zinc finger nuclease comprises the nucleotide sequence of SEQ ID NO: 22. In some embodiments, the polynucleotide sequence encoding the first zinc finger nuclease comprises the nucleotide sequence of SEQ ID NO: 23. In some embodiments, the polynucleotide sequence encoding the first zinc finger nuclease comprises the nucleotide sequence of SEQ ID NO: 25. In some embodiments, the polynucleotide sequence encoding the first zinc finger nuclease comprises the nucleotide sequence of SEQ ID NO: 26. In some embodiments, the polynucleotide sequence encoding the first zinc finger nuclease comprises the nucleotide sequence of SEQ ID NO: 27. In some embodiments, the polynucleotide sequence encoding the first zinc finger nuclease comprises the nucleotide sequence of SEQ ID NO: 28. In some embodiments, the polynucleotide sequence encoding the first zinc finger nuclease comprises the nucleotide sequence of SEQ ID NO: 29. In some embodiments, the polynucleotide sequence encoding the first zinc finger nuclease comprises the nucleotide sequence of SEQ ID NO: 30. In some embodiments, the polynucleotide sequence encoding the first zinc finger nuclease comprises the nucleotide sequence of SEQ ID NO: 31.

In some embodiments, the polynucleotide sequence encoding the second zinc finger nuclease comprises the nucleotide sequence of any one of SEQ ID NOs: 17-23 and 25-31. In some embodiments, the polynucleotide sequence encoding the second zinc finger nuclease comprises the nucleotide sequence of SEQ ID NO: 17. In some embodiments, the polynucleotide sequence encoding the second zinc finger nuclease comprises the nucleotide sequence of SEQ ID NO: 18. In some embodiments, the polynucleotide sequence encoding the second zinc finger nuclease comprises the nucleotide sequence of SEQ ID NO: 19. In some embodiments, the polynucleotide sequence encoding the second zinc finger nuclease comprises the nucleotide sequence of SEQ ID NO: 20. In some embodiments, the polynucleotide sequence encoding the second zinc finger nuclease comprises the nucleotide sequence of SEQ ID NO: 21. In some embodiments, the polynucleotide sequence encoding the second zinc finger nuclease comprises the nucleotide sequence of SEQ ID NO: 22. In some embodiments, the polynucleotide sequence encoding the second zinc finger nuclease comprises the nucleotide sequence of SEQ ID NO: 23. In some embodiments, the polynucleotide sequence encoding the second zinc finger nuclease comprises the nucleotide sequence of SEQ ID NO: 25. In some embodiments, the polynucleotide sequence encoding the second zinc finger nuclease comprises the nucleotide sequence of SEQ ID NO: 26. In some embodiments, the polynucleotide sequence encoding the second zinc finger nuclease comprises the nucleotide sequence of SEQ ID NO: 27. In some embodiments, the polynucleotide sequence encoding the second zinc finger nuclease comprises the nucleotide sequence of SEQ ID NO: 28. In some embodiments, the polynucleotide sequence encoding the second zinc finger nuclease comprises the nucleotide sequence of SEQ ID NO: 29. In some embodiments, the polynucleotide sequence encoding the second zinc finger nuclease comprises the nucleotide sequence of SEQ ID NO: 30. In some embodiments, the polynucleotide sequence encoding the second zinc finger nuclease comprises the nucleotide sequence of SEQ ID NO: 31.

A “2A sequence” or “2A self-cleaving sequence”, as used herein, refers to any sequence that encodes a peptide which can induce the cleaving a recombinant protein in a cell. In some embodiments the nucleotide sequence encoding the 2A self-cleaving sequence encodes a peptide that is between 15 and 25 amino acids. In some embodiments the nucleotide sequence encoding the 2A self-cleaving sequence encodes a peptide that is between 18 and 22 amino acids. Non-limiting examples of 2A self-cleaving peptides include T2A, P2A, E2A and F2A sequences. See, e.g., Donnelly et al. (2001) J. Gen. Virol. 82:1013-1025.

In some embodiments, the nucleotide sequence encoding the 2A self-cleaving sequence comprises the nucleotide sequence of SEQ ID NO:24. In some embodiments the nucleotide sequence encodes a 2A self-cleaving sequence comprising the amino acid sequence of SEQ ID NO: 138.

In some embodiments, the nucleic acid encoding a 2-in-1 zinc finger nuclease variant comprises a nucleotide selected from any one of SEQ ID NO: 85-115. In some embodiments, the nucleic acid encoding a 2-in-1 zinc finger nuclease variant comprises the nucleotide sequence of SEQ ID NO: 85. In some embodiments, the nucleic acid encoding a 2-in-1 zinc finger nuclease variant comprises the nucleotide sequence of SEQ ID NO: 86. In some embodiments, the nucleic acid encoding a 2-in-1 zinc finger nuclease variant comprises the nucleotide sequence of SEQ ID NO: 87. In some embodiments, the nucleic acid encoding a 2-in-1 zinc finger nuclease variant comprises the nucleotide sequence of SEQ ID NO: 88. In some embodiments, the nucleic acid encoding a 2-in-1 zinc finger nuclease variant comprises the nucleotide sequence of SEQ ID NO: 89. In some embodiments, the nucleic acid encoding a 2-in-1 zinc finger nuclease variant comprises the nucleotide sequence of SEQ ID NO: 90. In some embodiments, the nucleic acid encoding a 2-in-1 zinc finger nuclease variant comprises the nucleotide sequence of SEQ ID NO: 91. In some embodiments, the nucleic acid encoding a 2-in-1 zinc finger nuclease variant comprises the nucleotide sequence of SEQ ID NO: 92. In some embodiments, the nucleic acid encoding a 2-in-1 zinc finger nuclease variant comprises the nucleotide sequence of SEQ ID NO: 93. In some embodiments, the nucleic acid encoding a 2-in-1 zinc finger nuclease variant comprises the nucleotide sequence of SEQ ID NO: 94. In some embodiments, the nucleic acid encoding a 2-in-1 zinc finger nuclease variant comprises the nucleotide sequence of SEQ ID NO: 95. In some embodiments, the nucleic acid encoding a 2-in-1 zinc finger nuclease variant comprises the nucleotide sequence of SEQ ID NO: 96. In some embodiments, the nucleic acid encoding a 2-in-1 zinc finger nuclease variant comprises the nucleotide sequence of SEQ ID NO: 97. In some embodiments, the nucleic acid encoding a 2-in-1 zinc finger nuclease variant comprises the nucleotide sequence of SEQ ID NO: 98. In some embodiments, the nucleic acid encoding a 2-in-1 zinc finger nuclease variant comprises the nucleotide sequence of SEQ ID NO: 99. In some embodiments, the nucleic acid encoding a 2-in-1 zinc finger nuclease variant comprises the nucleotide sequence of SEQ ID NO: 100. In some embodiments, the nucleic acid encoding a 2-in-1 zinc finger nuclease variant comprises the nucleotide sequence of SEQ ID NO: 101. In some embodiments, the nucleic acid encoding a 2-in-1 zinc finger nuclease variant comprises the nucleotide sequence of SEQ ID NO: 102. In some embodiments, the nucleic acid encoding a 2-in-1 zinc finger nuclease variant comprises the nucleotide sequence of SEQ ID NO: 103. In some embodiments, the nucleic acid encoding a 2-in-1 zinc finger nuclease variant comprises the nucleotide sequence of SEQ ID NO: 104. In some embodiments, the nucleic acid encoding a 2-in-1 zinc finger nuclease variant comprises the nucleotide sequence of SEQ ID NO: 105. In some embodiments, the nucleic acid encoding a 2-in-1 zinc finger nuclease variant comprises the nucleotide sequence of SEQ ID NO: 106. In some embodiments, the nucleic acid encoding a 2-in-1 zinc finger nuclease variant comprises the nucleotide sequence of SEQ ID NO: 107. In some embodiments, the nucleic acid encoding a 2-in-1 zinc finger nuclease variant comprises the nucleotide sequence of SEQ ID NO: 108. In some embodiments, the nucleic acid encoding a 2-in-1 zinc finger nuclease variant comprises the nucleotide sequence of SEQ ID NO: 109. In some embodiments, the nucleic acid encoding a 2-in-1 zinc finger nuclease variant comprises the nucleotide sequence of SEQ ID NO: 110. In some embodiments, the nucleic acid encoding a 2-in-1 zinc finger nuclease variant comprises the nucleotide sequence of SEQ ID NO: 111. In some embodiments, the nucleic acid encoding a 2-in-1 zinc finger nuclease variant comprises the nucleotide sequence of SEQ ID NO: 112. In some embodiments, the nucleic acid encoding a 2-in-1 zinc finger nuclease variant comprises the nucleotide sequence of SEQ ID NO: 113. In some embodiments, the nucleic acid encoding a 2-in-1 zinc finger nuclease variant comprises the nucleotide sequence of SEQ ID NO: 114. In some embodiments, the nucleic acid encoding a 2-in-1 zinc finger nuclease variant comprises the nucleotide sequence of SEQ ID NO: 115.

In some embodiments, the nucleic acid encoding a 2-in-1 zinc finger nuclease variant comprises a nucleotide sequence selected from any one of SEQ ID NO: 35-49. In some embodiments, the nucleic acid encoding a 2-in-1 zinc finger nuclease variant comprises a nucleotide sequence selected from any one of SEQ ID NO: 35. In some embodiments, the nucleic acid encoding a 2-in-1 zinc finger nuclease variant comprises a nucleotide sequence selected from any one of SEQ ID NO: 36. In some embodiments, the nucleic acid encoding a 2-in-1 zinc finger nuclease variant comprises a nucleotide sequence selected from any one of SEQ ID NO: 37. In some embodiments, the nucleic acid encoding a 2-in-1 zinc finger nuclease variant comprises a nucleotide sequence selected from any one of SEQ ID NO: 35-38. In some embodiments, the nucleic acid encoding a 2-in-1 zinc finger nuclease variant comprises a nucleotide sequence selected from any one of SEQ ID NO: 39. In some embodiments, the nucleic acid encoding a 2-in-1 zinc finger nuclease variant comprises a nucleotide sequence selected from any one of SEQ ID NO: 40. In some embodiments, the nucleic acid encoding a 2-in-1 zinc finger nuclease variant comprises a nucleotide sequence selected from any one of SEQ ID NO: 41. In some embodiments, the nucleic acid encoding a 2-in-1 zinc finger nuclease variant comprises a nucleotide sequence selected from any one of SEQ ID NO: 42. In some embodiments, the nucleic acid encoding a 2-in-1 zinc finger nuclease variant comprises a nucleotide sequence selected from any one of SEQ ID NO: 43. In some embodiments, the nucleic acid encoding a 2-in-1 zinc finger nuclease variant comprises a nucleotide sequence selected from any one of SEQ ID NO: 44. In some embodiments, the nucleic acid encoding a 2-in-1 zinc finger nuclease variant comprises a nucleotide sequence selected from any one of SEQ ID NO: 45. In some embodiments, the nucleic acid encoding a 2-in-1 zinc finger nuclease variant comprises a nucleotide sequence selected from any one of SEQ ID NO: 46. In some embodiments, the nucleic acid encoding a 2-in-1 zinc finger nuclease variant comprises a nucleotide sequence selected from any one of SEQ ID NO: 47. In some embodiments, the nucleic acid encoding a 2-in-1 zinc finger nuclease variant comprises a nucleotide sequence selected from any one of SEQ ID NO: 48. In some embodiments, the nucleic acid encoding a 2-in-1 zinc finger nuclease variant comprises a nucleotide sequence selected from any one of SEQ ID NO: 49.

In some embodiments, the nucleic acid encoding a 2-in-1 zinc finger nuclease variant comprises a nucleotide encoding the amino acid sequence set forth in any one of SEQ ID NO: 132-135. In some embodiments, the nucleic acid encoding a 2-in-1 zinc finger nuclease variant comprises a nucleotide encoding the amino acid sequence set forth in SEQ ID NO: 132. In some embodiments, the nucleic acid encoding a 2-in-1 zinc finger nuclease variant comprises a nucleotide encoding the amino acid sequence set forth in SEQ ID NO: 133. In some embodiments, the nucleic acid encoding a 2-in-1 zinc finger nuclease variant comprises a nucleotide encoding the amino acid sequence set forth in SEQ ID NO: 134. In some embodiments, the nucleic acid encoding a 2-in-1 zinc finger nuclease variant comprises a nucleotide encoding the amino acid sequence set forth in SEQ ID NO: 135.

In some embodiments, the nucleic acid encoding a 2-in-1 zinc finger nuclease variant further comprises one or more 5′ITR, enhancer, promoter, 5′UTR, intron, post-transcriptional regulatory element, polyadenylation signal, or 3′ITR or any combination thereof. Each of the one or more 5′ITR, 3′ITR, enhancer, promoter, 5′UTR, 3′UTR, intron, post-transcriptional regulatory element, polyadenylation signal, and is independently operatively linked to the polynucleotide encoding the first and second ZFPs. Examples of such sequences are in Table 4.

In some embodiments, the nucleic acid encoding a 2-in-1 zinc finger nuclease variant further comprises one or more inverted terminal repeat (ITR) sequences. ITR are comprised of a nucleotide sequence that is followed by its reverse complement. Examples of inverted repeats include direct repeats, tandem repeats and palindromes. The ITR may be 5′ITR, a 3′ITR or both. The ITRs play a role in the integration of the viral construct into the host genome and rescue the viral construct from the host genome.

In some embodiments, the nucleic acid sequence encoding the 2-in-1 zinc finger nuclease variant further comprises a 5′ITR. In some embodiments, the 5′ITR comprises the nucleotide sequence set forth in SEQ ID NO: 10. In some embodiments, the nucleic acid sequence encoding the 2-in-1 zinc finger nuclease variant further comprises a 3′ITR. In some embodiments, the 3′ITR comprise the nucleotide sequence set forth in SEQ ID NO: 34. In some embodiments, the nucleic acid sequence encoding a 2-in-1 zinc finger nuclease variant further comprises an enhancer. In some embodiments, the enhancer is a eukaryotic enhancer. In some embodiments, the enhancer is a liver-specific enhancer. In some embodiments, the enhancer is a prokaryotic enhancer. In some embodiments the enhancer may be a viral enhancer. Exemplary enhancers include alpha 1 microglobulin/bikunin enhancer, SV40, CMV, HBV, and apolipoprotein E (ApoE). An exemplary liver-specific enhancer includes apolipoprotein E (APOE).

In some embodiments, the enhancer comprises a liver-specific enhancer. In some embodiments, the enhancer comprises an APOE enhancer. In some embodiments, the enhancer comprises the nucleotide sequence set forth in SEQ ID NO: 11.

In some embodiments, the nucleic acid sequence encoding the 2-in-1 zinc finger nuclease variant further comprises a promoter. In some embodiments, the promoter is a eukaryotic promoter. In some embodiments, the promoter is a prokaryotic promoter. In some embodiments, the promoter is a viral promoter. In some embodiments, the promoter is a liver-specific promoter. Exemplary promoters include CMV, CMVP, EF1a, CAG, PGK, TRE, U6, UAS, SV40, 5′LTR, polyhedron promoter (PH), TK, RSV, adenoviral E1A, human alpha 1-antitrypsin (hAAT), murine albumin (mAlb), phosphoenolpyruvate carboxykinase (rPECK), rat liver fatty acid binding protein, minimal transthyretin (TTR), thyroxine-binding globulin (TBG), EF1a, PGK1, Ubc, human beta-actin, CAG, Ac5, CaMKIIa, GAL1, GAL10, TEF1, GDS, ADH1, CaMV35S, Ubi, H1, U6, HBV and the like. Exemplary viral promoters include CMV, SV40, 5′LTR, PH, TK, RSV, adenoviral E1A, CaMV35S, HBV and the like. Exemplary liver-specific promoters include human alpha 1-antitrypsin (hAAT), murine albumin (mAlb), phosphoenolpyruvate carboxykinase (rPECK), rat liver fatty acid binding protein, minimal transthyretin (TTR), thyroxine-binding globulin (TBG) and the like.

In some embodiments, the promoter comprises a hAAT promoter. In some embodiments, the promoter comprises the nucleotide sequence set forth in SEQ ID NO: 12.

In some embodiments, the nucleic acid sequence encoding the 2-in-1 zinc finger nuclease variant further comprises a UTR sequence. The UTR may be a 5′ UTR, a 3′UTR or both. In some embodiments, the nucleic acid sequence encoding the 2-in-1 zinc finger nuclease variant comprises a 5′UTR. In some embodiments, the nucleic acid sequence encoding the 2-in-1 zinc finger nuclease variant comprises a 3′UTR. In some embodiments, the nucleic acid sequence encoding the 2-in-1 zinc finger nuclease variant comprises a 5′UTR and a 3′UTR. In some embodiments, the 5′UTR comprises the nucleotide sequence set forth in SEQ ID NO: 13.

In some embodiments, the nucleic acid sequence encoding the 2-in-1 zinc finger nuclease variant further comprises a chimeric intron. Chimeric intron refers to an intronic regulatory element engineered into a polynucleotide construct. Chimeric introns have been reported to enhance mRNA processing (i.e. splicing), increase expression levels of downstream open reading frames, increase expression of weak promoters, and increase duration of expression in vivo. Exemplary chimeric intron includes Human β-globin/IgG chimeric intron. In some embodiments, the chimeric intron comprises a Human β-globin/IgG chimeric intron. In some embodiments, the chimeric intron comprises the nucleotide sequence set forth in SEQ ID NO: 14.

In some embodiments, the nucleic acid sequence encoding the 2-in-1 zinc finger nuclease variant further comprises a post-transcriptional regulatory element. Exemplary post-transcriptional regulatory elements include Woodchuck hepatitis virus post-transcriptional regulatory element (WPRE) and hepatitis B post-transcriptional regulatory element (HPRE). WPRE is a 600 bp long tripartite element containing gamma, alpha, and beta elements, in the given order, (Donello et al. (1992) J Virol 72:5085-5092) and contributes to the strong expression of transgenes in AAV systems (Loeb et al. (1999) Hum Gene Ther 10:2295-2305). It also enhances the expression of a transgene lacking introns. In its natural form, WPRE contains a partial open reading frame (ORF) for the WHV-X protein. The fully expressed WHV-X protein, in the context of other viral elements like the WHV (We2) enhancer, has been associated with a higher risk of hepatocarcinoma in woodchucks and mice (Hohne et. al (1990) EMBO J 9(4):1137-45; Flajolet et. al (1998) J Virol 72(7):6175-80). The WHV-X protein does not appear to be directly oncogenic, but some studies suggest that under certain circumstances it can act as a weak cofactor for the generation of liver cancers associated with infection by hepadnaviruses (hepatitis B virus for man; woodchuck hepatitis virus for woodchucks). “Wildtype” WPRE refers to a 591 bp sequence (nucleotides 1094-1684 in GenBank accession number J02442) containing a portion of the WHV X protein open-reading frame (ORF) in its 3′ region. A “mutated” WPRE sequence (i.e. WPREmut6) refers to a WPRE sequence that lacks the transcription of a fragment of the potentially oncogenic woodchuck hepatitis virus-X protein. In this element, there is an initial ATG start codon for WHV-X at position 1502 and a promoter region with the sequence GCTGA at position 1488. In Zanta-Boussif (ibid), a mut6WPRE sequence was disclosed wherein the promoter sequence at position 1488 was modified to ATCA T and the start codon at position 1502 was modified to TTG, effectively prohibiting expression of WHV-X. In the J04514.1 WPRE variant, the ATG WHV X start site is a position 1504, and a mut6 type variant can be made in the J04514.1 strain. Another WPRE variant is the 247 bp WPRE3 variant comprising only minimal gamma and alpha elements from the wild type WPRE (Choi et al. (2014) Mol Brain 7:17), which lacks the WHV X sequences. A WPRE sequence (e.g., WRPEmut6 variant) from J02442.1 may also be used.

In some embodiments, the nucleic acid sequence encoding the 2-in-1 zinc finger nuclease variant comprises a 3′ WPRE sequence (see U.S. Patent Publication No. 2016/0326548). In some embodiments, the WPRE is a wild type WPRE. In some embodiments, the WPRE element is a mutated in the ‘X’ region to prevent expression of Protein X (see U.S. Pat. No. 7,419,829). In some embodiments, the mutated WPRE element comprises mutations described in Zanta-Boussif et al. (2009) Gene Ther 16(5):605-619, for example a WPREmut6 sequence. In some embodiments, the WPRE is a WPRE3 variant (Choi et al. (2014) Mol Brain 7:17). In some embodiments, the WPRE comprises a WPREmut6. In some embodiments, the WPRE comprises the nucleotide sequence set forth in SEQ ID NO: 32.

In some embodiments, the nucleic acid sequence encoding the 2-in-1 zinc finger nuclease variant further comprises a polyadenylation (poly A) signal. Exemplary polyadenylation signals include bovine Growth Hormone (bGH), human Growth Hormone (hGH), SV40, and rbGlob. In some embodiments, the poly A signal comprises a bGH poly A signal. In some embodiments the poly A signal comprises a hGH poly A signal. In some embodiments, the poly A signal comprises an SV40 poly A signal. In some embodiments, the poly A signal comprises a rbGlob poly A signal. In some embodiments, the poly A signal comprises the nucleotide sequence set forth in SEQ ID NO: 33.

In some embodiments, the 2-in-1 zinc finger nuclease variant nucleic acid sequence of the disclosure comprises at least about 60%, about 65%, about 70%, about 75%, about 80%, about 85%, about 90%, about 91%, about 92%, about 93%, about 94%, about 95%, about 96%, about 97%, about 98%, about 99% or more sequence identity to any of the sequences disclosed herein, as determined by sequence alignment programs known by skilled artisans.

Thus, in addition to the sequences encoding the components of the paired nuclease, the constructs may include additional coding or non-coding sequences in any order or combination. Constructs include constructs in which the left ZFN coding sequence is 5′ to the right ZFN coding sequence and constructs in which the right ZFN-encoding sequence is 5′ the left ZFN coding sequence. One or both of the left or right ZFN encoding sequences may be codon diversified in any way. The term “single diversified constructs” refers to constructs in which one ZFN (either left or right in any order in the construct) is encoded by a diversified sequence. The term “dual diversified constructs” refers to constructs in which both the left and right ZFNs (in any order in the construct) are codon diversified.

In some embodiments, the compositions and methods disclose herein comprise a 2-in-1 zinc finger nuclease variant. In some embodiments, the 2-in-1 zinc finger nuclease variant comprises a first zinc finger nuclease and a second zinc finger nuclease separated by a 2A self-cleaving peptide positioned in between the first zinc finger nuclease and the second zinc finger nuclease. In some embodiments, the first zinc finger nuclease is codon diversified. In some embodiments, the first zinc finger nuclease is not codon diversified. In some embodiments the second zinc finger nuclease is codon diversified. In some embodiments the second zinc finger nuclease is not codon diversified. In some embodiments, the first zinc finger nuclease and the second zinc finger nuclease are each independently codon diversified. In some embodiments, neither the first zinc finger nuclease nor the second zinc finger nuclease is codon diversified.

In some embodiments, the 2-in-1 zinc finger nuclease variant further comprises a) one or nuclear localization sequences; b) one or more epitope tag; and c) one or more cleavage domains.

In some embodiments, the first zinc finger nuclease comprises the amino acid sequence of any one of SEQ ID NOs: 136-137. In some embodiments, the first zinc finger nuclease comprises the amino acid sequence of SEQ ID NOs: 136. In some embodiments, the first zinc finger nuclease comprises the amino acid sequence of SEQ ID NOs: 137.

In some embodiments, the first zinc finger nuclease is encoded by a polynucleotide comprising the nucleotide sequence set forth in any one of SEQ ID NOs: 116-129. In some embodiments, the first zinc finger nuclease is encoded by a polynucleotide comprising the nucleotide sequence set forth in SEQ ID NO: 116. In some embodiments, the first zinc finger nuclease is encoded by a polynucleotide comprising the nucleotide sequence set forth in SEQ ID NO: 117. In some embodiments, the first zinc finger nuclease is encoded by a polynucleotide comprising the nucleotide sequence set forth in SEQ ID NO: 118. In some embodiments, the first zinc finger nuclease is encoded by a polynucleotide comprising the nucleotide sequence set forth in SEQ ID NO: 119. In some embodiments, the first zinc finger nuclease is encoded by a polynucleotide comprising the nucleotide sequence set forth in SEQ ID NO: 120. In some embodiments, the first zinc finger nuclease is encoded by a polynucleotide comprising the nucleotide sequence set forth in SEQ ID NO: 121. In some embodiments, the first zinc finger nuclease is encoded by a polynucleotide comprising the nucleotide sequence set forth in SEQ ID NO: 122. In some embodiments, the first zinc finger nuclease is encoded by a polynucleotide comprising the nucleotide sequence set forth in SEQ ID NO: 123. In some embodiments, the first zinc finger nuclease is encoded by a polynucleotide comprising the nucleotide sequence set forth in SEQ ID NO: 124. In some embodiments, the first zinc finger nuclease is encoded by a polynucleotide comprising the nucleotide sequence set forth in SEQ ID NO: 125. In some embodiments, the first zinc finger nuclease is encoded by a polynucleotide comprising the nucleotide sequence set forth in SEQ ID NO: 126. In some embodiments, the first zinc finger nuclease is encoded by a polynucleotide comprising the nucleotide sequence set forth in SEQ ID NO: 127. In some embodiments, the first zinc finger nuclease is encoded by a polynucleotide comprising the nucleotide sequence set forth in SEQ ID NO: 128. In some embodiments, the first zinc finger nuclease is encoded by a polynucleotide comprising the nucleotide sequence set forth in SEQ ID NO: 129.

In some embodiments, the second zinc finger nuclease comprises the amino acid sequence of any one of SEQ ID NOs: 136-137. In some embodiments, the second zinc finger nuclease comprises the amino acid sequence of SEQ ID NOs: 136. In some embodiments, the second zinc finger nuclease comprises the amino acid sequence of SEQ ID NOs: 137.

In some embodiments, the second zinc finger nuclease is encoded by a polynucleotide comprising the nucleotide sequence set forth in any one of SEQ ID NOs: 116-129. In some embodiments, the second zinc finger nuclease is encoded by a polynucleotide comprising the nucleotide sequence set forth in SEQ ID NO: 116. In some embodiments, the second zinc finger nuclease is encoded by a polynucleotide comprising the nucleotide sequence set forth in SEQ ID NO: 117. In some embodiments, the second zinc finger nuclease is encoded by a polynucleotide comprising the nucleotide sequence set forth in SEQ ID NO: 118. In some embodiments, the second zinc finger nuclease is encoded by a polynucleotide comprising the nucleotide sequence set forth in SEQ ID NO: 119. In some embodiments, the second zinc finger nuclease is encoded by a polynucleotide comprising the nucleotide sequence set forth in SEQ ID NO: 120. In some embodiments, the second zinc finger nuclease is encoded by a polynucleotide comprising the nucleotide sequence set forth in SEQ ID NO: 121. In some embodiments, the second zinc finger nuclease is encoded by a polynucleotide comprising the nucleotide sequence set forth in SEQ ID NO: 122. In some embodiments, the second zinc finger nuclease is encoded by a polynucleotide comprising the nucleotide sequence set forth in SEQ ID NO: 123. In some embodiments, the second zinc finger nuclease is encoded by a polynucleotide comprising the nucleotide sequence set forth in SEQ ID NO: 124. In some embodiments, the second zinc finger nuclease is encoded by a polynucleotide comprising the nucleotide sequence set forth in SEQ ID NO: 125. In some embodiments, the second zinc finger nuclease is encoded by a polynucleotide comprising the nucleotide sequence set forth in SEQ ID NO: 126. In some embodiments, the second zinc finger nuclease is encoded by a polynucleotide comprising the nucleotide sequence set forth in SEQ ID NO: 127. In some embodiments, the second zinc finger nuclease is encoded by a polynucleotide comprising the nucleotide sequence set forth in SEQ ID NO: 128. In some embodiments, the second zinc finger nuclease is encoded by a polynucleotide comprising the nucleotide sequence set forth in SEQ ID NO: 129.

In some embodiments, the 2-in-1 zinc finger nuclease variant further comprises one or more cleavage domains. Any suitable cleavage domain can be associated with (e.g., operatively linked) to a zinc finger DNA-binding domain (e.g., ZFP). Each of the cleavage domains may have the same amino acid sequence. Alternatively, they each of the cleavage domains may have a different amino acid sequence. In some embodiments, the cleavage domain comprises a Fok I cleavage domain, which is active as a dimer. In some embodiments the nucleotide sequence encoding the one or more Fok I cleavage domain is codon diversified. In some embodiments the nucleotide sequence encoding the one or more Fok I cleavage domain is not codon diversified. In some embodiments a first Fok I cleavage domain is operatively linked to the first zinc finger DNA binding protein (ZFP). In some embodiments a second Fok I cleavage domain is operatively linked to the second zinc finger DNA binding protein (ZFP). In some embodiments the first Fok I cleavage domain is located 3′ to the first zinc finger DNA binding protein (ZFP). In some embodiments the second Fok I cleavage domain is located 3′ to the second zinc finger DNA binding protein (ZFP).

In some embodiments, the first zinc finger nuclease comprises the amino acid sequence of any one of SEQ ID NOs: 130-131. In some embodiments, the first zinc finger nuclease comprises the amino acid sequence of SEQ ID NOs: 130. In some embodiments, the first zinc finger nuclease comprises the amino acid sequence of SEQ ID NOs: 131.

In some embodiments, the second zinc finger nuclease comprises the amino acid sequence of any one of SEQ ID NOs: 130-131. In some embodiments, the second zinc finger nuclease comprises the amino acid sequence of SEQ ID NOs: 130. In some embodiments, the second zinc finger nuclease comprises a nucleotide sequence encoding the amino acid sequence of SEQ ID NOs: 131.

In some embodiments, the first zinc finger nuclease is encoded by a polynucleotide sequence comprising the nucleotide sequence set forth in any one of SEQ ID NOs: 71-84. In some embodiments, the first zinc finger nuclease is encoded by a polynucleotide comprising the nucleotide sequence as set forth in SEQ ID NO: 71. In some embodiments, the first zinc finger nuclease is encoded by a polynucleotide comprising the nucleotide sequence as set forth in SEQ ID NO: 72. In some embodiments, the first zinc finger nuclease is encoded by a polynucleotide comprising the nucleotide sequence as set forth in SEQ ID NO: 73. In some embodiments, the first zinc finger nuclease is encoded by a polynucleotide comprising the nucleotide sequence as set forth in SEQ ID NO: 74. In some embodiments, the first zinc finger nuclease is encoded by a polynucleotide comprising the nucleotide sequence as set forth in SEQ ID NO: 75. In some embodiments, the first zinc finger nuclease is encoded by a polynucleotide comprising the nucleotide sequence as set forth in SEQ ID NO: 76. In some embodiments, the first zinc finger nuclease is encoded by a polynucleotide comprising the nucleotide sequence as set forth in SEQ ID NO: 77. In some embodiments, the first zinc finger nuclease is encoded by a polynucleotide comprising the nucleotide sequence as set forth in SEQ ID NO: 78. In some embodiments, the first zinc finger nuclease is encoded by a polynucleotide comprising the nucleotide sequence as set forth in SEQ ID NO: 79. In some embodiments, the first zinc finger nuclease is encoded by a polynucleotide comprising the nucleotide sequence as set forth in SEQ ID NO: 80. In some embodiments, the first zinc finger nuclease is encoded by a polynucleotide comprising the nucleotide sequence as set forth in SEQ ID NO: 81. In some embodiments, the first zinc finger nuclease is encoded by a polynucleotide comprising the nucleotide sequence as set forth in SEQ ID NO: 82. In some embodiments, the first zinc finger nuclease is encoded by a polynucleotide comprising the nucleotide sequence as set forth in SEQ ID NO: 83. In some embodiments, the first zinc finger nuclease is encoded by a polynucleotide comprising the nucleotide sequence as set forth in SEQ ID NO: 84.

In some embodiments, the second zinc finger nuclease is encoded by a polynucleotide sequence comprising the nucleotide sequence set forth in any one of SEQ ID NOs: 71-84. In some embodiments, the second zinc finger nuclease is encoded by a polynucleotide comprising the nucleotide sequence as set forth in SEQ ID NO: 71. In some embodiments, the second zinc finger nuclease is encoded by a polynucleotide comprising the nucleotide sequence as set forth in SEQ ID NO: 72. In some embodiments, the second zinc finger nuclease is encoded by a polynucleotide comprising the nucleotide sequence as set forth in SEQ ID NO: 73. In some embodiments, the second zinc finger nuclease is encoded by a polynucleotide comprising the nucleotide sequence as set forth in SEQ ID NO: 74. In some embodiments, the second zinc finger nuclease is encoded by a polynucleotide comprising the nucleotide sequence as set forth in SEQ ID NO: 75. In some embodiments, the second zinc finger nuclease is encoded by a polynucleotide comprising the nucleotide sequence as set forth in SEQ ID NO: 76. In some embodiments, the second zinc finger nuclease is encoded by a polynucleotide comprising the nucleotide sequence as set forth in SEQ ID NO: 77. In some embodiments, the second zinc finger nuclease is encoded by a polynucleotide comprising the nucleotide sequence as set forth in SEQ ID NO: 78. In some embodiments, the second zinc finger nuclease is encoded by a polynucleotide comprising the nucleotide sequence as set forth in SEQ ID NO: 79. In some embodiments, the second zinc finger nuclease is encoded by a polynucleotide comprising the nucleotide sequence as set forth in SEQ ID NO: 80. In some embodiments, the second zinc finger nuclease is encoded by a polynucleotide comprising the nucleotide sequence as set forth in SEQ ID NO: 81. In some embodiments, the second zinc finger nuclease is encoded by a polynucleotide comprising the nucleotide sequence as set forth in SEQ ID NO: 82. In some embodiments, the second zinc finger nuclease is encoded by a polynucleotide comprising the nucleotide sequence as set forth in SEQ ID NO: 83. In some embodiments, the second zinc finger nuclease is encoded by a polynucleotide comprising the nucleotide sequence as set forth in SEQ ID NO: 84.

In some embodiments, the zinc finger nuclease further comprises one or more nuclear localization sequence (NLS). Each of the NLS may have the same amino acid sequence. Alternatively, each NLS may have a different amino acid sequence. In some embodiments, the zinc finger nuclease comprises a first nuclear localization sequence (NLS) and a second nuclear localization sequence (NLS), wherein the first nuclear localization sequence (NLS) is located N-terminal (i.e., upstream) to the first zinc finger DNA binding protein (ZFP) and the second nuclear localization sequence (NLS) is located N-terminal (i.e., upstream) to the second zinc finger DNA binding protein (ZFP). In some embodiments, the first NLS is operatively linked to the first ZFP and the second NLS is operatively linked to the second ZFP. In some embodiments, the first NLS is codon diversified. In some embodiments, the first NLS is not codon diversified. In some embodiments, the second NLS is codon diversified. In some embodiments, the second NLS is not codon diversified.

In some embodiments, the first NLS comprises the amino acid sequence set forth in any one of SEQ ID NO: 3-9 and 156. In some embodiments, the first NLS comprises a nucleotide sequence encoding the amino acid sequence set forth in SEQ ID NO: 3. In some embodiments, the first NLS comprises the amino acid sequence set forth in SEQ ID NO: 4. In some embodiments, the first NLS comprises the amino acid sequence set forth in SEQ ID NO:5. In some embodiments, the first NLS comprises the amino acid sequence set forth in SEQ ID NO: 6. In some embodiments, the first NLS comprises the amino acid sequence set forth in SEQ ID NO: 7. In some embodiments, the first NLS comprises the amino acid sequence set forth in SEQ ID NO: 8. In some embodiments, the first NLS comprises the amino acid sequence set forth in SEQ ID NO: 9. In some embodiments, the first NLS comprises the amino acid sequence set forth in SEQ ID NO: 156. In some embodiments, the second NLS comprises the amino acid sequence set forth in any one of SEQ ID NO: 3-9 and 156. In some embodiments, the second NLS comprises the amino acid sequence set forth in SEQ ID NO: 3. In some embodiments, the second NLS comprises the amino acid sequence set forth in SEQ ID NO: 4. In some embodiments, the second NLS comprises the amino acid sequence set forth in SEQ ID NO:5. In some embodiments, the second NLS comprises the amino acid sequence set forth in SEQ ID NO: 6. In some embodiments, the second NLS comprises the amino acid sequence set forth in SEQ ID NO: 7. In some embodiments, the second NLS comprises the amino acid sequence set forth in SEQ ID NO: 8. In some embodiments, the second NLS comprises the amino acid sequence set forth in SEQ ID NO: 9. In some embodiments, the second NLS comprises the amino acid sequence set forth in SEQ ID NO: 156.

In some embodiments, the first NLS is encoded by the nucleotide sequence set forth in any one of SEQ ID NO: 59-70. In some embodiments, the first NLS is encoded by a nucleotide sequence comprising the nucleotide sequence set forth in SEQ ID NO: 59. In some embodiments, the first NLS is encoded by a nucleotide sequence comprising the nucleotide sequence set forth in SEQ ID NO: 60. In some embodiments, the first NLS is encoded by a nucleotide sequence comprising the nucleotide sequence set forth in SEQ ID NO: 61. In some embodiments, the first NLS is encoded by a nucleotide sequence comprising the nucleotide sequence set forth in SEQ ID NO: 62. In some embodiments, the first NLS is encoded by a nucleotide sequence comprising the nucleotide sequence set forth in SEQ ID NO: 63. In some embodiments, the first NLS is encoded by a nucleotide sequence comprising the nucleotide sequence set forth in SEQ ID NO: 64 In some embodiments, the first NLS is encoded by a nucleotide sequence comprising the nucleotide sequence set forth in SEQ ID NO: 65. In some embodiments, the first NLS is encoded by a nucleotide sequence comprising the nucleotide sequence set forth in SEQ ID NO: 66. In some embodiments, the first NLS is encoded by a nucleotide sequence comprising the nucleotide sequence set forth in SEQ ID NO: 67. In some embodiments, the first NLS is encoded by a nucleotide sequence comprising the nucleotide sequence set forth in SEQ ID NO: 68. In some embodiments, the first NLS is encoded by a nucleotide sequence comprising the nucleotide sequence set forth in SEQ ID NO: 69. In some embodiments, the first NLS is encoded by a nucleotide sequence comprising the nucleotide sequence set forth in SEQ ID NO: 70.

In some embodiments, the second NLS is encoded by the nucleotide sequence set forth in any one of SEQ ID NO: 59-70. In some embodiments, the second NLS is encoded by a nucleotide sequence comprising the nucleotide sequence set forth in SEQ ID NO: 59. In some embodiments, the second NLS is encoded by a nucleotide sequence comprising the nucleotide sequence set forth in SEQ ID NO: 60. In some embodiments, the second NLS is encoded by a nucleotide sequence comprising the nucleotide sequence set forth in SEQ ID NO: 61. In some embodiments, the second NLS is encoded by a nucleotide sequence comprising the nucleotide sequence set forth in SEQ ID NO: 62. In some embodiments, the second NLS is encoded by a nucleotide sequence comprising the nucleotide sequence set forth in SEQ ID NO: 63. In some embodiments, the second NLS is encoded by a nucleotide sequence comprising the nucleotide sequence set forth in SEQ ID NO: 64 In some embodiments, the second NLS is encoded by a nucleotide sequence comprising the nucleotide sequence set forth in SEQ ID NO: 65. In some embodiments, the second NLS is encoded by a nucleotide sequence comprising the nucleotide sequence set forth in SEQ ID NO: 66. In some embodiments, the second NLS is encoded by a nucleotide sequence comprising the nucleotide sequence set forth in SEQ ID NO: 67. In some embodiments, the second NLS is encoded by a nucleotide sequence comprising the nucleotide sequence set forth in SEQ ID NO: 68. In some embodiments, the second NLS is encoded by a nucleotide sequence comprising the nucleotide sequence set forth in SEQ ID NO: 69. In some embodiments, the second NLS is encoded by a nucleotide sequence comprising the nucleotide sequence set forth in SEQ ID NO: 70.

In some embodiments, the 2-in-1 zinc finger nuclease variant further comprises one or more epitope tag. Epitope tags include, for example one or more copies of FLAG, HA, CBP, GST, HBH, MBP, Myc, His, polyHis, S-tag, SUMO, TAP, TAGP, TRX, V5, GFP, RFP, YFP, and the like.

In some embodiments, the 2-in-1 zinc finger nuclease variant further comprises one or one or more copies of a epitope tag. In some embodiments, the 2-in-1 zinc finger nuclease variant comprises a first epitope tag and a second epitope tag. In some embodiments, each of said first epitope tag and second epitope tag is the same. In some embodiments, each of said first epitope tag and second epitope tag are different. In some embodiments, the first epitope tag is located N-terminal to the first ZFP, and the second epitope tag is located N-terminal to the second ZFP. In some embodiments, the first epitope tag is located N-terminal to the first NLS, and the second epitope tag is located N terminal to the second NLS. In some embodiments, the first epitope tag is located C-terminal to the first ZFP, and the second epitope tag is located C-terminal to the second ZFP. In some embodiments, the first epitope tag is located C-terminal to the first NLS, and the second epitope tag is located C-terminal to the second NLS. In some embodiments, the first epitope tag is codon diversified. In some embodiments, the first epitope tag is not codon diversified. In some embodiments, the second epitope tag is codon diversified. In some embodiments, the second epitope tag is not codon diversified.

In some embodiments, the 2-in-1 zinc finger nuclease variant further comprises one or one or more copies of a FLAG tag. In some embodiments, the epitope tag is 3×FLAG. In some embodiments, the 2-in-1 zinc finger nuclease variant comprises a first FLAG tag and a second FLAG tag. In some embodiments, each of said first FLAG tag and second FLAG tag is 3×FLAG. In some embodiments, the first FLAG tag is located N-terminal to the first ZFP, and the second FLAG tag is located N-terminal to the second ZFP. In some embodiments, the first FLAG tag is located N-terminal to the first NLS, and the second FLAG tag is located N terminal to the second NLS. In some embodiments, the first FLAG tag is located C-terminal to the first ZFP, and the second FLAG tag is located C-terminal to the second ZFP. In some embodiments, the first FLAG tag is located C-terminal to the first NLS, and the second FLAG tag is located C-terminal to the second NLS. In some embodiments, the first FLAG tag is codon diversified. In some embodiments, the first FLAG tag is not codon diversified. In some embodiments, the second FLAG tag is codon diversified. In some embodiments, the second FLAG tag is not codon diversified.

In some embodiments, the first FLAG tag comprises the amino acid sequence set forth in any one of SEQ ID NO: 1-2. In some embodiments, the first FLAG tag comprises the amino acid sequence set forth in SEQ ID NO: 1. In some embodiments, the first FLAG tag comprises the amino acid sequence set forth in SEQ ID NO: 2. In some embodiments, the second FLAG tag comprises the amino acid sequence set forth in any one of SEQ ID NO: 1-2. In some embodiments, the second FLAG tag comprises the amino acid sequence set forth in SEQ ID NO: 1. In some embodiments, the second FLAG tag comprises the amino acid sequence set forth in SEQ ID NO: 2.

In some embodiments, the first FLAG tag is encoded by a polynucleotide comprising the nucleotide sequence set forth in any one of SEQ ID NO: 15-16, 50-58, 153 or 154. In some embodiments, the first FLAG tag is encoded by a polynucleotide comprising the nucleotide sequence set forth in any one of SEQ ID NO: 15. In some embodiments, the first FLAG tag is encoded by a polynucleotide comprising the nucleotide sequence set forth in any one of SEQ ID NO: 16. In some embodiments, the first FLAG tag is encoded by a polynucleotide comprising the nucleotide sequence set forth in any one of SEQ ID NO: 50. In some embodiments, the first FLAG tag is encoded by a polynucleotide comprising the nucleotide sequence set forth in any one of SEQ ID NO: 51. In some embodiments, the first FLAG tag is encoded by a polynucleotide comprising the nucleotide sequence set forth in any one of SEQ ID NO: 52. In some embodiments, the first FLAG tag is encoded by a polynucleotide comprising the nucleotide sequence set forth in any one of SEQ ID NO: 53. In some embodiments, the first FLAG tag is encoded by a polynucleotide comprising the nucleotide sequence set forth in any one of SEQ ID NO: 54. In some embodiments, the first FLAG tag is encoded by a polynucleotide comprising the nucleotide sequence set forth in any one of SEQ ID NO: 55. In some embodiments, the first FLAG tag is encoded by a polynucleotide comprising the nucleotide sequence set forth in any one of SEQ ID NO: 56. In some embodiments, the first FLAG tag is encoded by a polynucleotide comprising the nucleotide sequence set forth in any one of SEQ ID NO: 57. In some embodiments, the first FLAG tag is encoded by a polynucleotide comprising the nucleotide sequence set forth in any one of SEQ ID NO: 58. In some embodiments, the second FLAG tag is encoded by a polynucleotide comprising the nucleotide sequence set forth in any one of SEQ ID NO: 153. In some embodiments, the second FLAG tag is encoded by a polynucleotide comprising the nucleotide sequence set forth in any one of SEQ ID NO: 154.

In some embodiments, the second FLAG tag is encoded by a polynucleotide comprising the nucleotide sequence set forth in any one of SEQ ID NO: 15-16, 50-58, 153 or 154. In some embodiments, the second FLAG tag is encoded by a polynucleotide comprising the nucleotide sequence set forth in any one of SEQ ID NO: 15. In some embodiments, the second FLAG tag is encoded by a polynucleotide comprising the nucleotide sequence set forth in any one of SEQ ID NO: 16. In some embodiments, the second FLAG tag is encoded by a polynucleotide comprising the nucleotide sequence set forth in any one of SEQ ID NO: 50. In some embodiments, the second FLAG tag is encoded by a polynucleotide comprising the nucleotide sequence set forth in any one of SEQ ID NO: 51. In some embodiments, the second FLAG tag is encoded by a polynucleotide comprising the nucleotide sequence set forth in any one of SEQ ID NO: 52. In some embodiments, the second FLAG tag is encoded by a polynucleotide comprising the nucleotide sequence set forth in any one of SEQ ID NO: 53. In some embodiments, the second FLAG tag is encoded by a polynucleotide comprising the nucleotide sequence set forth in any one of SEQ ID NO: 54. In some embodiments, the second FLAG tag is encoded by a polynucleotide comprising the nucleotide sequence set forth in any one of SEQ ID NO: 55. In some embodiments, the second FLAG tag is encoded by a polynucleotide comprising the nucleotide sequence set forth in any one of SEQ ID NO: 56. In some embodiments, the second FLAG tag is encoded by a polynucleotide comprising the nucleotide sequence set forth in any one of SEQ ID NO: 57. In some embodiments, the second FLAG tag is encoded by a polynucleotide comprising the nucleotide sequence set forth in any one of SEQ ID NO: 58. In some embodiments, the second FLAG tag is encoded by a polynucleotide comprising the nucleotide sequence set forth in any one of SEQ ID NO: 153. In some embodiments, the second FLAG tag is encoded by a polynucleotide comprising the nucleotide sequence set forth in any one of SEQ ID NO: 154.

In some embodiments, the first zinc finger nuclease is encoded by a polynucleotide comprising the nucleotide sequence of any one of SEQ ID NOs: 17-23 and 25-31. In some embodiments, the first zinc finger nuclease is encoded by a polynucleotide comprising the nucleotide sequence of SEQ ID NO: 17. In some embodiments, the first zinc finger nuclease is encoded by a polynucleotide comprising the nucleotide sequence of SEQ ID NO: 18. In some embodiments, the first zinc finger nuclease is encoded by a polynucleotide comprising the nucleotide sequence of SEQ ID NO: 19. In some embodiments, the first zinc finger nuclease is encoded by a polynucleotide comprising the nucleotide sequence of SEQ ID NO: 20. In some embodiments, the first zinc finger nuclease is encoded by a polynucleotide comprising the nucleotide sequence of SEQ ID NO: 21. In some embodiments, the first zinc finger nuclease is encoded by a polynucleotide comprising the nucleotide sequence of SEQ ID NO: 22. In some embodiments, the first zinc finger nuclease is encoded by a polynucleotide comprising the nucleotide sequence of SEQ ID NO: 23. In some embodiments, the first zinc finger nuclease is encoded by a polynucleotide comprising the nucleotide sequence of SEQ ID NO: 25. In some embodiments, the first zinc finger nuclease is encoded by a polynucleotide comprising the nucleotide sequence of SEQ ID NO: 26. In some embodiments, the first zinc finger nuclease is encoded by a polynucleotide comprising the nucleotide sequence of SEQ ID NO: 27. In some embodiments, the first zinc finger nuclease is encoded by a polynucleotide comprising the nucleotide sequence of SEQ ID NO: 28. In some embodiments, the first zinc finger nuclease is encoded by a polynucleotide comprising the nucleotide sequence of SEQ ID NO: 29. In some embodiments, the first zinc finger nuclease is encoded by a polynucleotide comprising the nucleotide sequence of SEQ ID NO: 30. In some embodiments, the first zinc finger nuclease is encoded by a polynucleotide comprising the nucleotide sequence of SEQ ID NO: 31.

In some embodiments, the second zinc finger nuclease is encoded by a polynucleotide comprising the nucleotide sequence of any one of SEQ ID NOs: 17-23 and 25-31. In some embodiments, the second zinc finger nuclease is encoded by a polynucleotide comprising the nucleotide sequence of SEQ ID NO: 17. In some embodiments, the second zinc finger nuclease is encoded by a polynucleotide comprising the nucleotide sequence of SEQ ID NO: 18. In some embodiments, the second zinc finger nuclease is encoded by a polynucleotide comprising the nucleotide sequence of SEQ ID NO: 19. In some embodiments, the second zinc finger nuclease is encoded by a polynucleotide comprising the nucleotide sequence of SEQ ID NO: 20. In some embodiments, the second zinc finger nuclease is encoded by a polynucleotide comprising the nucleotide sequence of SEQ ID NO: 21. In some embodiments, the second zinc finger nuclease is encoded by a polynucleotide comprising the nucleotide sequence of SEQ ID NO: 22. In some embodiments, the second zinc finger nuclease is encoded by a polynucleotide comprising the nucleotide sequence of SEQ ID NO: 23. In some embodiments, the second zinc finger nuclease is encoded by a polynucleotide comprising the nucleotide sequence of SEQ ID NO: 25. In some embodiments, the second zinc finger nuclease is encoded by a polynucleotide comprising the nucleotide sequence of SEQ ID NO: 26. In some embodiments, the second zinc finger nuclease is encoded by a polynucleotide comprising the nucleotide sequence of SEQ ID NO: 27. In some embodiments, the second zinc finger nuclease is encoded by a polynucleotide comprising the nucleotide sequence of SEQ ID NO: 28. In some embodiments, the second zinc finger nuclease is encoded by a polynucleotide comprising the nucleotide sequence of SEQ ID NO: 29. In some embodiments, the second zinc finger nuclease is encoded by a polynucleotide comprising the nucleotide sequence of SEQ ID NO: 30. In some embodiments, the second zinc finger nuclease is encoded by a polynucleotide comprising the nucleotide sequence of SEQ ID NO: 31.

In some embodiments the 2A self-cleaving peptide is between 15 and 25 amino acids. In some embodiments the 2A self-cleaving peptide is between 18 and 22 amino acids. Non-limiting examples of 2A self-cleaving peptides include T2A, P2A, E2A and F2A sequences. See, e.g., Donnelly et al. (2001) J. Gen. Virol. 82:1013-1025. In some embodiments the 2A self-cleaving sequence comprises the amino acid sequence of SEQ ID NO: 138. In some embodiments, the 2A self-cleaving sequence is encoded by a polynucleotide comprising the nucleotide sequence of SEQ ID NO:24.

In some embodiments, the 2-in-1 zinc finger nuclease variant comprises the amino acid sequence set forth in any one of SEQ ID NO: 132-135. In some embodiments, the 2-in-1 zinc finger nuclease variant comprises the amino acid sequence set forth in SEQ ID NO: 132. In some embodiments, the 2-in-1 zinc finger nuclease variant comprises the amino acid sequence set forth in SEQ ID NO: 133. In some embodiments, the 2-in-1 zinc finger nuclease variant comprises the amino acid sequence set forth in SEQ ID NO: 134. In some embodiments, the 2-in-1 zinc finger nuclease variant comprises the amino acid sequence set forth in SEQ ID NO: 135.

In some embodiments, the 2-in-1 zinc finger nuclease variant is encoded by a nucleic acid comprising a nucleotide sequence selected from any one of SEQ ID NO: 85-115. In some embodiments, the 2-in-1 zinc finger nuclease variant is encoded by a nucleic acid comprising the nucleotide sequence set forth in SEQ ID NO: 85. In some embodiments, the 2-in-1 zinc finger nuclease variant is encoded by a nucleic acid comprising the nucleotide sequence set forth in SEQ ID NO: 86. In some embodiments, the 2-in-1 zinc finger nuclease variant is encoded by a nucleic acid comprising the nucleotide sequence set forth in SEQ ID NO: 87. In some embodiments, the 2-in-1 zinc finger nuclease variant is encoded by a nucleic acid comprising the nucleotide sequence set forth in SEQ ID NO: 88. In some embodiments, the 2-in-1 zinc finger nuclease variant is encoded by a nucleic acid comprising the nucleotide sequence set forth in SEQ ID NO: 89. In some embodiments, the 2-in-1 zinc finger nuclease variant is encoded by a nucleic acid comprising the nucleotide sequence set forth in SEQ ID NO: 90. In some embodiments, the 2-in-1 zinc finger nuclease variant is encoded by a nucleic acid comprising the nucleotide sequence set forth in SEQ ID NO: 91. In some embodiments, the 2-in-1 zinc finger nuclease variant is encoded by a nucleic acid comprising the nucleotide sequence set forth in SEQ ID NO: 92. In some embodiments, the 2-in-1 zinc finger nuclease variant is encoded by a nucleic acid comprising the nucleotide sequence set forth in SEQ ID NO: 93. In some embodiments, the 2-in-1 zinc finger nuclease variant is encoded by a nucleic acid comprising the nucleotide sequence set forth in SEQ ID NO: 94. In some embodiments, the 2-in-1 zinc finger nuclease variant is encoded by a nucleic acid comprising the nucleotide sequence set forth in SEQ ID NO: 95. In some embodiments, the 2-in-1 zinc finger nuclease variant is encoded by a nucleic acid comprising the nucleotide sequence set forth in SEQ ID NO: 96. In some embodiments, the 2-in-1 zinc finger nuclease variant is encoded by a nucleic acid comprising the nucleotide sequence set forth in SEQ ID NO: 97. In some embodiments, the 2-in-1 zinc finger nuclease variant is encoded by a nucleic acid comprising the nucleotide sequence set forth in SEQ ID NO: 98. In some embodiments, the 2-in-1 zinc finger nuclease variant is encoded by a nucleic acid comprising the nucleotide sequence set forth in SEQ ID NO: 99. In some embodiments, the 2-in-1 zinc finger nuclease variant is encoded by a nucleic acid comprising the nucleotide sequence set forth in SEQ ID NO: 100. In some embodiments, the 2-in-1 zinc finger nuclease variant is encoded by a nucleic acid comprising the nucleotide sequence set forth in SEQ ID NO: 101. In some embodiments, the 2-in-1 zinc finger nuclease variant is encoded by a nucleic acid comprising the nucleotide sequence set forth in SEQ ID NO: 102. In some embodiments, the 2-in-1 zinc finger nuclease variant is encoded by a nucleic acid comprising the nucleotide sequence set forth in SEQ ID NO: 103. In some embodiments, the 2-in-1 zinc finger nuclease variant is encoded by a nucleic acid comprising the nucleotide sequence set forth in SEQ ID NO: 104 In some embodiments, the 2-in-1 zinc finger nuclease variant is encoded by a nucleic acid comprising the nucleotide sequence set forth in SEQ ID NO: 105. In some embodiments, the 2-in-1 zinc finger nuclease variant is encoded by a nucleic acid comprising the nucleotide sequence set forth in SEQ ID NO: 106 In some embodiments, the 2-in-1 zinc finger nuclease variant is encoded by a nucleic acid comprising the nucleotide sequence set forth in SEQ ID NO: 107. In some embodiments, the 2-in-1 zinc finger nuclease variant is encoded by a nucleic acid comprising the nucleotide sequence set forth in SEQ ID NO: 108 In some embodiments, the 2-in-1 zinc finger nuclease variant is encoded by a nucleic acid comprising the nucleotide sequence set forth in SEQ ID NO: 109. In some embodiments, the 2-in-1 zinc finger nuclease variant is encoded by a nucleic acid comprising the nucleotide sequence set forth in SEQ ID NO: 110 In some embodiments, the 2-in-1 zinc finger nuclease variant is encoded by a nucleic acid comprising the nucleotide sequence set forth in SEQ ID NO: 111. In some embodiments, the 2-in-1 zinc finger nuclease variant is encoded by a nucleic acid comprising the nucleotide sequence set forth in SEQ ID NO: 112. In some embodiments, the 2-in-1 zinc finger nuclease variant is encoded by a nucleic acid comprising the nucleotide sequence set forth in SEQ ID NO: 113. In some embodiments, the 2-in-1 zinc finger nuclease variant is encoded by a nucleic acid comprising the nucleotide sequence set forth in SEQ ID NO: 114. In some embodiments, the 2-in-1 zinc finger nuclease variant is encoded by a nucleic acid comprising the nucleotide sequence set forth in SEQ ID NO: 115.

In some embodiments, the 2-in-1 zinc finger nuclease variant of the disclosure comprises at least about 60%, about 65%, about 70%, about 75%, about 80%, about 85%, about 90%, about 91%, about 92%, about 93%, about 94%, about 95%, about 96%, about 97%, about 98%, about 99% or more sequence identity to any of the sequences disclosed herein, as determined by sequence alignment programs known by skilled artisans.

In some embodiments, the 2-in-1 zinc finger nuclease variant comprising a first zinc finger nuclease and a second zinc finger nuclease separated by a 2A self-cleaving peptide positioned in between the first zinc finger nuclease and the second zinc finger nuclease is encoded by a polynucleotide comprising the nucleotide sequence as set forth in SEQ ID NOs: 100-115.

### Methods of Using Push-Pull Donor Constructs

The polynucleotide constructs, vectors and pharmaceutical compositions disclosed herein may be used in a variety of methods.

In one aspect, the present disclosure provides a method for modifying the genome of a cell, the method comprising introducing into the cell a push-pull donor polynucleotide construct of the disclosure, a vector of the disclosure or a pharmaceutical composition of the disclosure. In some embodiments, the present disclosure provides a method for modifying the genome of a cell, the method comprising introducing into the cell the push-pull donor polynucleotide constructs of the disclosure. In some embodiments, the present disclosure provides a method for modifying the genome of a cell, the method comprising introducing into the cell the vectors of the disclosure. In some embodiments, the present disclosure provides a method for modifying the genome of a cell, the method comprising introducing into the cell a pharmaceutical composition of the disclosure.

In some embodiments, the method for modifying the genome of a cell comprises introducing into the cell a push-pull donor polynucleotide construct of the disclosure, a first polynucleotide encoding a first zinc finger nuclease, and a second polynucleotide encoding a second zinc finger nuclease. In some embodiments, the method for modifying the genome of a cell comprises introducing into the cell a push-pull donor polynucleotide construct of the disclosure and a polynucleotide encoding one or more zinc finger nucleases. In some embodiments, the method for modifying the genome of a cell comprises introducing into the cell a push-pull donor polynucleotide construct of the disclosure and a polynucleotide encoding a 2-in-1 zinc finger nuclease.

In some embodiments, the method for modifying the genome of a cell comprises introducing into the cell a vector comprising a push-pull donor polynucleotide construct of the disclosure, a vector comprising first polynucleotide encoding a first zinc finger nuclease, and vector comprising a second polynucleotide encoding a second zinc finger nuclease. In some embodiments, the method for modifying the genome of a cell comprises introducing into the cell a vector comprising a push-pull donor polynucleotide construct of the disclosure, and a vector comprising a polynucleotide encoding one or more zinc finger nuclease. In some embodiments, the method for modifying the genome of a cell comprises introducing into the cell a vector comprising a push-pull donor polynucleotide construct of the disclosure and a vector comprising a polynucleotide encoding a 2-in-1 zinc finger nuclease.

In some embodiments, the method for modifying the genome of a cell comprises introducing into the cell a pharmaceutical composition comprising a push-pull donor polynucleotide construct of the disclosure, a first polynucleotide encoding a first zinc finger nuclease, and a second polynucleotide encoding a second zinc finger nuclease. In some embodiments, the method for modifying the genome of a cell comprises introducing into the cell a pharmaceutical composition comprising a push-pull donor polynucleotide construct of the disclosure and a polynucleotide encoding one or more zinc finger nuclease. In some embodiments, the method for modifying the genome of a cell comprises introducing into the cell a pharmaceutical composition comprising a push-pull donor polynucleotide construct of the disclosure and a polynucleotide encoding a 2-in-1 zinc finger nuclease.

In another aspect, the present disclosure provides a method for integrating an exogenous nucleotide sequence or transgene into a target nucleotide sequence in a gene of a cell, the method comprising introducing into a cell a push-pull donor polynucleotide construct of the disclosure, a vector of the disclosure or a pharmaceutical compositions of the disclosure. In some embodiments, the present disclosure provides a method for integrating an exogenous nucleotide sequence or transgene into a target nucleotide sequence in a gene of a cell, the method comprising introducing into a cell a push-pull donor polynucleotide construct of the disclosure. In some embodiments, the present disclosure provides a method for integrating an exogenous nucleotide sequence or transgene into a target nucleotide sequence in a gene of a cell, the method comprising introducing into a cell a vector of the disclosure. In some embodiments, the present disclosure provides a method for integrating an exogenous nucleotide sequence or transgene into a target nucleotide sequence in a gene of a cell, the method comprising introducing into a cell a pharmaceutical composition of the disclosure.

In some embodiments, the method for integrating an exogenous nucleotide sequence or transgene into a target nucleotide sequence in a gene of a cell comprises introducing into the cell a push-pull donor polynucleotide construct of the disclosure, a first polynucleotide encoding a first zinc finger nuclease, and a second polynucleotide encoding a second zinc finger nuclease. In some embodiments, the method for integrating an exogenous nucleotide sequence or transgene into a target nucleotide sequence in a gene of a cell comprises introducing into the cell a push-pull donor polynucleotide construct of the disclosure and a polynucleotide encoding one or more zinc finger nuclease. In some embodiments, the method for integrating an exogenous nucleotide sequence or transgene into a target nucleotide sequence in a gene of a cell comprises introducing into the cell a push-pull donor polynucleotide construct of the disclosure and a polynucleotide encoding a 2-in-1 zinc finger nuclease.

In some embodiments, the method for integrating an exogenous nucleotide sequence or transgene into a target nucleotide sequence in a gene of a cell comprises introducing into the cell a vector comprising a push-pull donors polynucleotide construct of the disclosure, a vector comprising first polynucleotide encoding a first zinc finger nuclease, and vector comprising a second polynucleotide encoding a second zinc finger nuclease. In some embodiments, the method for integrating an exogenous nucleotide sequence or transgene into a target nucleotide sequence in a gene of a cell comprises introducing into the cell a vector comprising a push-pull donor polynucleotide construct of the disclosure, and a vector comprising a polynucleotide encoding one or more zinc finger nuclease. In some embodiments, the method for integrating an exogenous nucleotide sequence or transgene into a target nucleotide sequence in a gene of a cell comprises introducing into the cell a vector comprising a push-pull donor polynucleotide construct of the disclosure and a vector comprising a polynucleotide encoding a 2-in-1 zinc finger nuclease.

In some embodiments, the method for integrating an exogenous nucleotide sequence or transgene into a target nucleotide sequence in a gene of a cell comprises introducing into the cell a pharmaceutical composition comprising a push-pull donor polynucleotide construct of the disclosure, a first polynucleotide encoding a first zinc finger nuclease, and a second polynucleotide encoding a second zinc finger nuclease. In some embodiments, the method for integrating an exogenous nucleotide sequence or transgene into a target nucleotide sequence in a gene of a cell comprises introducing into the cell a pharmaceutical composition comprising a push-pull donor polynucleotide construct of the disclosure and a polynucleotide encoding one or more zinc finger nuclease. In some embodiments, the method for integrating an exogenous nucleotide sequence or transgene into a target nucleotide sequence in a gene of a cell comprises introducing into the cell a pharmaceutical composition comprising a push-pull donor polynucleotide construct of the disclosure and a polynucleotide encoding a 2-in-1 zinc finger nuclease.

In another aspect, the present disclosure provides a method for disrupting a target nucleotide sequence in a cell, the method comprising introducing into a cell a push-pull donor polynucleotide construct of the disclosure, a vector of the disclosure or a pharmaceutical composition of the disclosure. In some embodiments, the present disclosure provides a method for disrupting a target nucleotide sequence in a cell, the method comprising introducing into a cell a push-pull donor polynucleotide construct of the disclosure. In some embodiments, the present disclosure provides a method disrupting a target nucleotide sequence in a cell, the method comprising introducing into a cell a vector of the disclosure. In some embodiments, the present disclosure provides a method for disrupting a target nucleotide sequence in a cell, the method comprising introducing into a cell a pharmaceutical composition of the disclosure.

In some embodiments, the method for disrupting a target nucleotide sequence in a cell comprises introducing into the cell a push-pull donor polynucleotide construct of the disclosure, a first polynucleotide encoding a first zinc finger nuclease, and a second polynucleotide encoding a second zinc finger nuclease. In some embodiments, the method for disrupting a target nucleotide sequence in a cell comprises introducing into the cell a push-pull donor polynucleotide construct of the disclosure and a polynucleotide encoding one or more zinc finger nuclease. In some embodiments, the method for disrupting a target nucleotide sequence in a cell comprises introducing into the cell a push-pull donor polynucleotide construct of the disclosure and a polynucleotide encoding a 2-in-1 zinc finger nuclease.

In some embodiments, the method for disrupting a target nucleotide sequence in a cell comprises introducing into the cell a vector comprising a push-pull donors polynucleotide construct of the disclosure, a vector comprising first polynucleotide encoding a first zinc finger nuclease, and vector comprising a second polynucleotide encoding a second zinc finger nuclease. In some embodiments, the method for disrupting a target nucleotide sequence in a cell comprises introducing into the cell a vector comprising a push-pull donor polynucleotide construct of the disclosure, and a vector comprising a polynucleotide encoding one or more zinc finger nuclease. In some embodiments, the method for disrupting a target nucleotide sequence in a cell comprises introducing into the cell a vector comprising a push-pull donor polynucleotide construct of the disclosure and a vector comprising a polynucleotide encoding a 2-in-1 zinc finger nuclease.

In some embodiments, the method for disrupting a target nucleotide sequence in a cell comprises introducing into the cell a pharmaceutical composition comprising a push-pull donor polynucleotide construct of the disclosure, a first polynucleotide encoding a first zinc finger nuclease, and a second polynucleotide encoding a second zinc finger nuclease. In some embodiments, the method for disrupting a target nucleotide sequence in a cell comprises introducing into the cell a pharmaceutical composition comprising a push-pull donor polynucleotide construct of the disclosure and a polynucleotide encoding one or more zinc finger nuclease. In some embodiments, the method for disrupting a target nucleotide sequence in a cell comprises introducing into the cell a pharmaceutical composition comprising a push-pull donor polynucleotide construct of the disclosure and a polynucleotide encoding a 2-in-1 zinc finger nuclease.

In another aspect, the present disclosure provides a method for treating a disorder in a subject, the method comprising modifying a target nucleotide sequence in the genome of a cell of said subject by introducing into the cell a push-pull donor polynucleotide construct of the disclosure, a vector of the disclosure or a pharmaceutical compositions of the disclosure. In some embodiments, the present disclosure provides a method for treating a disorder in a subject, the method comprising modifying a target nucleotide sequence in the genome of a cell of said subject by introducing into the cell a push-pull donor polynucleotide construct of the disclosure. In some embodiments, the present disclosure provides a method for treating a disorder in a subject, the method comprising modifying a target nucleotide sequence in the genome of a cell of said subject by introducing into the cell a vector of the disclosure. In some embodiments, the present disclosure provides a method for treating a disorder in a subject, the method comprising modifying a target nucleotide sequence in the genome of a cell of said subject by introducing into the cell a cell a pharmaceutical composition of the disclosure.

In some embodiments, the method for treating a disorder in a subject comprises introducing into the cell of a subject a push-pull donor polynucleotide construct of the disclosure, a first polynucleotide encoding a first zinc finger nuclease, and a second polynucleotide encoding a second zinc finger nuclease. In some embodiments, the method for treating a disorder in a subject comprises introducing into the cell of a subject a push-pull donor polynucleotide construct of the disclosure and a polynucleotide encoding one or more zinc finger nuclease. In some embodiments, the method for treating a disorder in a subject comprises introducing into the cell of a subject a push-pull donor polynucleotide construct of the disclosure and a polynucleotide encoding a 2-in-1 zinc finger nuclease.

In some embodiments, the method for treating a disorder in a subject comprises introducing into the cell of a subject a vector comprising a push-pull donors polynucleotide construct of the disclosure, a vector comprising first polynucleotide encoding a first zinc finger nuclease, and vector comprising a second polynucleotide encoding a second zinc finger nuclease. In some embodiments, the method for treating a disorder in a subject comprises introducing into the cell of a subject a vector comprising a push-pull donor polynucleotide construct of the disclosure, and a vector comprising a polynucleotide encoding one or more zinc finger nuclease. In some embodiments, the method for treating a disorder in a subject comprises introducing into the cell of a subject a vector comprising a push-pull donor polynucleotide construct of the disclosure and a vector comprising a polynucleotide encoding a 2-in-1 zinc finger nuclease.

In some embodiments, the method for treating a disorder in a subject comprises introducing into the cell of a subject a pharmaceutical composition comprising a push-pull donor polynucleotide construct of the disclosure, a first polynucleotide encoding a first zinc finger nuclease, and a second polynucleotide encoding a second zinc finger nuclease. In some embodiments, the method for treating a disorder in a subject comprises introducing into the cell of a subject a pharmaceutical composition comprising a push-pull donor polynucleotide construct of the disclosure and a polynucleotide encoding one or more zinc finger nuclease. In some embodiments, the method for treating a disorder in a subject comprises introducing into the cell of a subject a pharmaceutical composition comprising a push-pull donor polynucleotide construct of the disclosure and a polynucleotide encoding a 2-in-1 zinc finger nuclease.

In another aspect, the present disclosure provides method for correcting a disease-causing mutation in the genome of a cell, the method comprising modifying a target nucleotide sequence in the genome of the cell by introducing into the cell an effective amount of a push-pull donor polynucleotide construct of the disclosure, a vector of the disclosure or a pharmaceutical compositions of the disclosure. In some embodiments, the present disclosure provides method for correcting a disease-causing mutation in the genome of a cell, the method comprising modifying a target nucleotide sequence in the genome of the cell by introducing into the cell an effective amount of a push-pull donor polynucleotide construct of the disclosure. In some embodiments, the present disclosure provides method for correcting a disease-causing mutation in the genome of a cell, the method comprising modifying a target nucleotide sequence in the genome of the cell by introducing into the cell an effective amount of a vector of the disclosure. In some embodiments, the present disclosure provides method for correcting a disease-causing mutation in the genome of a cell, the method comprising modifying a target nucleotide sequence in the genome of the cell by introducing into the cell an effective amount of a pharmaceutical composition of the disclosure.

In some embodiments, the method for correcting a disease-causing mutation in the genome of a cell comprises introducing into the cell of a subject a push-pull donor polynucleotide construct of the disclosure, a first polynucleotide encoding a first zinc finger nuclease, and a second polynucleotide encoding a second zinc finger nuclease. In some embodiments, the method for correcting a disease-causing mutation in the genome of a cell comprises introducing into the cell of a subject a push-pull donor polynucleotide construct of the disclosure and a polynucleotide encoding one or more zinc finger nuclease. In some embodiments, the method for correcting a disease-causing mutation in the genome of a cell comprises introducing into the cell of a subject a push-pull donor polynucleotide construct of the disclosure and a polynucleotide encoding a 2-in-1 zinc finger nuclease.

In some embodiments, the method for correcting a disease-causing mutation in the genome of a cell comprises introducing into the cell of a subject a vector comprising a push-pull donors polynucleotide construct of the disclosure, a vector comprising first polynucleotide encoding a first zinc finger nuclease, and vector comprising a second polynucleotide encoding a second zinc finger nuclease. In some embodiments, the method for correcting a disease-causing mutation in the genome of a cell comprises introducing into the cell of a subject a vector comprising a push-pull donor polynucleotide construct of the disclosure, and a vector comprising a polynucleotide encoding one or more zinc finger nuclease. In some embodiments, t the method for correcting a disease-causing mutation in the genome of a cell comprises introducing into the cell of a subject a vector comprising a push-pull donor polynucleotide construct of the disclosure and a vector comprising a polynucleotide encoding a 2-in-1 zinc finger nuclease.

In some embodiments, the method for correcting a disease-causing mutation in the genome of a cell comprises introducing into the cell of a subject a pharmaceutical composition comprising a push-pull donor polynucleotide construct of the disclosure, a first polynucleotide encoding a first zinc finger nuclease, and a second polynucleotide encoding a second zinc finger nuclease. In some embodiments, the method for correcting a disease-causing mutation in the genome of a cell comprises introducing into the cell of a subject a pharmaceutical composition comprising a push-pull donor polynucleotide construct of the disclosure and a polynucleotide encoding one or more zinc finger nuclease. In some embodiments, the method for correcting a disease-causing mutation in the genome of a cell comprises introducing into the cell of a subject a pharmaceutical composition comprising a push-pull donor polynucleotide construct of the disclosure and a polynucleotide encoding a 2-in-1 zinc finger nuclease.

In the methods disclosed herein, when the push-pull donor polynucleotide construct sequence integrates into a genomic locus, the polynucleotide can integrate in two orientations, but only one of the two nucleotides encoding a polypeptide is expressed (i.e., transcribed and/or translated). Thus, when the donor polynucleotide integrates in a first orientation, the first nucleotide sequence is expressed after being integrated into a genomic locus. When the donor polynucleotide integrates in a second orientation, the second nucleotide sequence is expressed after being integrated into a genomic locus. Thus, in some embodiments, the method further comprises the expression of the first nucleotide encoding the first polypeptide. In other embodiments, the method further comprises the expression of the second nucleotide encoding the second polypeptide.

A variety of diseases or disorders may be treated by employing the methods disclosed herein. Non-limiting examples of diseases or disorders include genetic disorders, infectious diseases, acquired disorders, cancer, and the like. Exemplary genetic disorders include achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangiectasia, barth syndrome, beta-thalassemia, blue rubber bleb nevus syndrome, canavan disease, chronic granulomatous diseases (CGD), citrullinemia, cri du chat syndrome, cystic fibrosis, dercum's disease, ectodermal dysplasia, Fabry disease, fanconi anemia, fibrodysplasia ossificans progressiva, fragile X syndrome, galactosemia, Gaucher's disease, generalized gangliosidoses (e.g., GM1), glycogen storage disease (e.g., GSD1), hemochromatosis, the hemoglobin C mutation in the 6th codon of beta-globin (HbC), hemophilia, Hunter syndrome, Huntington's disease, Hurler Syndrome, hypophosphatasia, Klinefelter syndrome, Krabbes Disease, Langer-Giedion Syndrome, leukocyte adhesion deficiency (LAD, OMIM No. 116920), leukodystrophy, long QT syndrome, lipoprotein lipase deficiency, Marfan syndrome, Moebius syndrome, mucopolysaccharidosis (MPS), nail patella syndrome, nephrogenic diabetes insipidus, neurofibromatosis, Niemann-Pick disease, ornithine transcarbamylase (OTC) deficiency, osteogenesis imperfecta, phenylketonuria (PKU), Pompe disease, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, and X-linked lymphoproliferative syndrome (XLP, OMIM No. 308240), and the like.

The methods disclosed herein also allow for treatment of infections (viral or bacterial) in a host (e.g., by blocking expression of viral or bacterial receptors, thereby preventing infection and/or spread in a host organism). Non-limiting examples of viruses or viral receptors that may be targeted include herpes simplex virus (HSV), such as HSV-1 and HSV-2, varicella zoster virus (VZV), Epstein-Barr virus (EBV) and cytomegalovirus (CMV), HHV6 and HHV7. The hepatitis family of viruses includes hepatitis A virus (HAV), hepatitis B virus (HBV), hepatitis C virus (HCV), the delta hepatitis virus (HDV), hepatitis E virus (HEV) and hepatitis G virus (HGV). Other viruses or their receptors may be targeted, including, but not limited to, Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; Retroviradae; lentiviruses (e.g., HTLV-I; HTLV-II; HIV-1 (also known as HTLV-III, LAV, ARV, hTLR, etc.) HIV-II); simian immunodeficiency virus (SIV), human papillomavirus (HPV), influenza virus and the tick-borne encephalitis viruses. See, e.g. Virology, 3rd Edition (W. K. Joklik ed. 1988); Fundamental Virology, 2nd Edition (B. N. Fields and D. M. Knipe, eds. 1991), for a description of these and other viruses. Also included are infections with other pathogenic organisms such as Mycobacterium Tuberculosis, Mycoplasma pneumoniae, and the like or parasites such as Plasmodium falciparum, and the like.

Genetic disease or disorders may also be treated or prevented using the methods disclosed herein. Exemplary genetic diseases that may be treated using the push-pull donor constructs and methods described herein include, but are not limited to, achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangiectasia, barth syndrome, beta-thalassemia, blue rubber bleb nevus syndrome, canavan disease, chronic granulomatous diseases (CGD), citrullinemia, cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), glycogen storage disease (e.g., GSD1), hemochromatosis, the hemoglobin C mutation in the 6th codon of beta-globin (HbC), hemophilia, Huntington's disease, Hurler Syndrome, hypophosphatasia, Klinefelter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Niemann-Pick disease, ornithine transcarbamylase (OTC) deficiency, osteogenesis imperfecta, phenylketonuria (PKU), porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240), and the like.

In some embodiments, the disclosure provides a method for treating a lysosomal storage disease in a subject, the method comprising modifying a target sequence in the genome of a cell of said subject using the push-pull donor constructs of the disclosure. In some embodiments, the disclosure provides a method for preventing a lysosomal storage disease in a subject, the method comprising modifying a target sequence in the genome of a cell of said subject using the push-pull donor constructs of the disclosure. In some embodiments, the method of treating or preventing a lysosomal storage disease includes improving or maintaining (slowing the decline) of functional ability in a human subject having a LSD. In some embodiments, the method of treating or preventing a lysosomal storage disease includes decreasing the need (dose level or frequency) for enzyme replacement therapy (ERT) in a subject with a LSD. In some embodiments, the method of treating or preventing a lysosomal storage disease includes delaying the need for ERT initiation in a subject with a LSD. In some embodiments, the method of treating or preventing a lysosomal storage disease includes delaying, reducing or eliminating the need for supportive surgery in a subject with a LSD (e.g., MPS II). In some embodiments, the method of treating or preventing a lysosomal storage disease includes delaying, reducing or preventing the need for a bone marrow transplant in a subject with a LSD In some embodiments, the method of treating or preventing a lysosomal storage disease includes improving the functional (delaying decline, maintenance) ability in a subject with a LSD. In some embodiments, the method of treating or preventing a lysosomal storage disease includes suppressing disability progression in a human subject having a LSD. In some embodiments, the method of treating or preventing a lysosomal storage disease includes delaying, reducing or preventing the need for the use of a medical ventilator device in a subject with a LSD. In some embodiments, the method of treating or preventing a lysosomal storage disease includes delaying onset of confirmed disability progression or reducing the risk of confirmed disability progression in a human subject having a LSD. In some embodiments, the method of treating or preventing a lysosomal storage disease includes reducing, stabilizing or maintaining urine GAGs in a subject with a LSD. In some embodiments, the method of treating or preventing a lysosomal storage disease includes extending life expectancy in a subject with a LSD.

In some embodiments, the disclosure provides a method for correcting a lysosomal storage disease-causing mutation in the genome of a cell using the push-pull donor constructs of the disclosure.

A variety of lysosomal storage diseases that may be treated and/or prevented by the methods disclosed herein. Exemplary lysosomal storage diseases that may be treated and/or prevented by 2-in-1 zinc finger nuclease variants described herein include, but are not limited to, Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/J/A), GM2 Tay-Sachs disease, GM2 Gangliosidosis AB variant, I-Cell Disease/Mtucolipidosis II, Krabbe Disease Lysosomal acid lipase deficiency, Metachromatic Leukodystrophy, MPS I—Hurler Syndrome, MPS I—Scheie Syndrome, MIPS I Hurler-Scheie Syndrome, MPS II Hunter Syndrome, MPS IIIA—Sanfilippo Syndrome Type A, MPS IIIB—Sanfilippo Syndrome Type B, MPS IIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuseinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, Wolman Disease and the like.

In some embodiments, a subject having MPS II may have attenuated form MPSII or severe MPS II. “Severe MPS II” in subjects is characterized by delayed speech and developmental delay between 18 months to 3 years of age. The disease is characterized in severe MPS II subjects by organomegaly, hyperactivity and aggressiveness, neurologic deterioration, joint stiffness and skeletal deformities (including abnormal spinal bones), coarse facial features with enlarged tongue, heart valve thickening, hearing loss and hernias. The life expectancy of untreated subjects with severe Hunter syndrome is into the mid teenage years with death due to neurologic deterioration and/or cardiorespiratory failure. “Attenuated form MPS II” in subjects are typically diagnosed later than the severe subjects. The somatic clinical features are similar to the severe subjects, but overall disease severity in milder with, in general, slower disease progression with no or only mild cognitive impairment. Death in the untreated attenuated form is often between the ages of 20-30 years from cardiac and respiratory disease.

The proteins associated with the various lysosomal storage diseases include, but are not limited to those set forth in Table 1.

TABLE 1

| LSD | Enzyme or protein | Gene |
|---|---|---|
| Alpha-mannosidosis | Alpha-D-mannosidase | MAN2B1 |
| Aspartylglucosaminuria | N-aspartyl-beta- | AGA |
| | glucosaminidase | |
| Cholesteryl ester storage | Lysosomal acid lipase | LIPA |
| disease | | |
| Cystinosis | Cystinosin | CTNS |
| Danon Disease | Lysosomal associated | LAMP2 |
| | membrane protein 2 | |
| Fabry Disease | Alpha-galactosidase A | GLA |
| Farber Disease | Acid ceramidase | ASAH1 |
| Fucosidosis | Alpha fucosidase | FUCA1 |
| Galactosialidosis | Cathepsin A | CTSA |
| Gaucher Disease Type I | Acid beta-glucocerebrosidase | GBA |
| Gaucher Disease Type II | Acid beta-glucocerebrosidase | GBA |
| Gaucher Disease Type III | Acid beta-glucocerebrosidase | GBA |
| GM1 Gangliosidosis | Beta galactosidase | GLB1 |
| (Types I, II and III) | | |
| GM2 Sandhoff Disease | Beta hexosaminidase A | HEXB |
| (I/J/A) | Beta hexosaminidase B | |
| GM2 Tay-Sachs disease | Beta-hexosaminidase | HEXA |
| GM2 Gangliosidosis AB | GM2 ganglioside activator | GM2A |
| variant | (GM2A) | |
| I-Cell Disease/ | GLcNAc-1-phospho- | GNPTAB |
| Mucolipidosis II | transferase | |
| Krabbe Disease | Beta-galactosylceramidase | GALC |
| Lysosomal acid lipase | Lysosomal acid lipase | LIPA |
| deficiency | | |
| Metachromatic | Aryl sulfatase A | ARSA |
| Leukodystrophy | | |
| MPS I—Hurler Syndrome | Alpha-L-iduronidase | IDUA |
| MPS I—Scheie Syndrome | Alpha-L-iduronidase | IDUA |
| MPS I Hurler-Scheie | Alpha-L-iduronidase | IDUA |
| Syndrome | | |
| MPS II Hunter Syndrome | Iduronate-2-sulphatase | IDS |
| MPS IIIA—Sanfilippo | Heparan N-sulfatase | SGSH |
| Syndrome Type A | | |
| MPS IIIB—Sanfilippo | Alpha-N-acetyl- | NAGLU |
| Syndrome Type B | glucosaminidase | |
| MPS IIIC—Sanfilippo | acetyl CoA:alpha- | GSNAT |
| Syndrome Type C | glucosaminide | |
| | acetyltransferase | |
| MPSIIID—Sanfilippo | N-acetyl glucosamine-6- | GNS |
| Syndrome Type D | sulfatase | |
| MPS IV—Morquio Type A | Galactosamine-6-sulfate | GALNS |
| | sulfatase | |
| MPS IV—Morquio Type B | Beta-galactosidase | GLB1 |
| MPS VI—Maroteaux-Lamy | Arylsulfatase B | ARSB |
| MPS VII—Sly Syndrome | Beta-glucuronidase | GUSB |
| MPS IX—Hyaluronidase | Hyaluronidase | HYAL1 |
| Deficiency | | |
| Mucolipidosis I—Sialidosis | Neuraminidase | NEU1 |
| Mucolipidosis IIIC | GlcNAc-1-phosphotransferase | GNPTG |
| Mucolipidosis Type IV | Mucolipin-1 | MCOLN1 |
| Multiple Sulfatase | Formylglycine-generating | SUMF1 |
| Deficiency | enzyme (FGE) | |
| Neuronal Ceroid | Palmitoyl-protein | PPT1 |
| Lipofuscinosis T1 | thioesterase 1 | |
| Neuronal Ceroid | tripeptidyl peptidase 1 | TPP1 |
| Lipofuscinosis T2 | | |
| Neuronal Ceroid | CLN3 protein | CLN3 |
| Lipofuscinosis T3 | | |
| Neuronal Ceroid | Cysteine string protein alpha | DNAJC5 |
| Lipofuscinosis T4 | | |
| Neuronal Ceroid | CLN5 protein | CLN5 |
| Lipofuscinosis T5 | | |
| Neuronal Ceroid | CLN6 protein | CLN6 |
| Lipofuscinosis T6 | | |
| Neuronal Ceroid | CLN7 protein | CLN7 |
| Lipofuscinosis T7 | | |
| Neuronal Ceroid | CLN8 protein | CLN8 |
| Lipofuscinosis T8 | | |
| Niemann-Pick Disease | Acid sphingomyelinase | SMPD1 |
| Type A | | |
| Niemann-Pick Disease | Acid sphingomyelinase | SMPD1 |
| Type B | | |
| Niemann-Pick Disease | NPC 1/NPC 2 | NPC1, |
| Type C | | NPC2 |
| Phenylketonuria | Phenylalanine hydroxylase | PAH |
| Pompe Disease | Acid alpha-glucosidase | GAA |
| Pycnodysostosis, | cathepsin K | CTSK |
| Sialic Acid Storage | Sialin (sialic acid transporter) | SLC17A5 |
| Disease | | |
| Schindler Disease | Alpha-N- | NAGA |
| | acetylgalactosaminidase | |
| Wolman Disease | Lysosomal acid lipase | LIPA |

Thus, in some embodiments, the methods disclosed herein comprise introducing into the cell a corrective disease-associated protein or enzyme or portion thereof. In some embodiments, the methods disclosed comprise introducing into the cell a push-pull donor polynucleotide construct encoding a corrective disease-associated protein or enzyme or portion thereof. In some embodiments, the methods disclosed herein comprise introducing into the cell a corrective disease-associated protein or enzyme as set forth in Table 1 or portions thereof. In some embodiments, the methods disclosed herein comprise introducing into the cell a corrective disease-associated gene as set forth in Table 1 or portions thereof.

In some embodiments, the methods disclosed herein comprise inserting one or more corrective disease-associated genes as set forth in Table 1 or portions thereof into a safe harbor locus (e.g. albumin) in a cell for expression of the needed protein(s) (e.g. enzyme(s) in Table 1) and release into the blood stream. Once in the bloodstream, the secreted enzyme may be taken up by cells in the tissues, wherein the enzyme is then taken up by the lysosomes such that the GAGs are broken down. In some embodiments, the inserted transgene encoding the disease associated protein (e.g., IDS, IDUA, GLA, GAA, PAH, etc.) is codon optimized. In some embodiments, the transgene is one in which the relevant epitope is removed without functionally altering the protein. In some embodiments, the methods comprise insertion of an episome expressing the corrective enzyme (or protein)-encoding transgene into a cell for expression of the needed enzyme and release into the blood stream. In some embodiments, the insertion is into a secretory cell, such as a liver cell for release of the product into the blood stream.

Subjects treatable using the methods of the invention include both humans and non-human animals.

The method for treatment or correction of a disease-causing mutation can take place in vivo or ex vivo. By “in vivo” it is meant in the living body of an animal. By “ex vivo” it is meant that cells or organs are modified outside of the body, such cells or organs are typically returned to a living body.

In some embodiments the methods disclosed herein comprise administering a vector comprising a push pull donor polynucleotide construct as disclosed herein at a dose of about 1×10⁹ vg/kg to about 1×10¹⁷ vg/kg. In some embodiments the dose of vector comprising a push pull donor polynucleotide construct as disclosed herein is about 1×10⁹ vg/kg, about 5×10⁹ vg/kg, about 1×10¹⁰ vg/kg, about 5×10¹⁰ vg/kg, about 1×10¹¹ vg/kg, about 5×10¹¹ vg/kg, about 1×10² vg/kg, about 5×10¹² vg/kg, about 1×10¹³ vg/kg, about 5×10¹³ vg/kg, about 1×10¹⁴ vg/kg, about 5×10¹⁴ vg/kg, about 1×10¹⁵ vg/kg, about 5×10¹⁵ vg/kg, about 1×10¹⁶ vg/kg, about 5×10¹⁶ vg/kg, about 1×10¹⁷ vg/kg. In some embodiments the dose of vector comprising a push pull donor polynucleotide construct as disclosed herein is 1×10⁹ vg/kg, 5×10⁹ vg/kg, 1×10¹⁰ vg/kg, 5×10¹⁰ vg/kg, 1×10¹¹ vg/kg, 5×10¹¹ vg/kg, 1×10² vg/kg, 5×10¹² vg/kg, 1×10¹³ vg/kg, 5×10¹³ vg/kg, 1×10¹⁴ vg/kg, 5×10¹⁴ vg/kg, 1×10¹⁵ vg/kg, 5×10¹⁵ vg/kg, 1×10¹⁶ vg/kg, 5×10¹⁶ vg/kg, 1×10¹⁷ vg/kg.

In some embodiments, the methods disclosed herein comprise administering a vector comprising a polynucleotide encoding one or more zinc finger nucleases at a dose of about 1×10¹² vg/kg to about 1×10¹⁶ vg/kg, about 1×10¹² vg/kg to about 1×10¹⁴ vg/kg. In some embodiments, the dose of vector comprising a polynucleotide encoding one or more zinc finger nucleases is about 1×10¹² vg/kg, about 5×10¹² vg/kg, about 1×10¹³ vg/kg, about 5×10¹³ vg/kg, about 1×10¹⁴ vg/kg, about 5×10¹⁴ vg/kg, about 1×10¹⁵ vg/kg, about 5×10¹⁵ vg/kg, about 1×10¹⁶ vg/kg, about 5×10¹⁶ vg/kg. In some embodiments, the dose of vector comprising a polynucleotide encoding one or more zinc finger nucleases is 1×10¹² vg/kg, 5×10¹² vg/kg, 1×10¹³ vg/kg, 5×10¹³ vg/kg, 1×10¹⁴ vg/kg, 5×10¹⁴ vg/kg, 1×10¹⁵ vg/kg, 5×10¹⁵ vg/kg, 1×10¹⁶ vg/kg, 5×10¹⁶ vg/kg. In some embodiments, the dose of vector comprising a polynucleotide encoding one or more zinc finger nucleases is about 1×10¹⁴ vg/kg. In some embodiments, the dose of vector comprising a polynucleotide encoding one or more zinc finger nucleases is 1×10¹⁴ vg/kg.

Methods for the therapeutic administration of vectors or constructs including the push-pull donor polynucleotide construct or polynucleotide encoding zinc finger nucleases of the disclosure are well known in the art. Nucleic acid constructs can be delivered with cationic lipids (Goddard, et al, Gene Therapy, 4:1231-1236, 1997; Gorman, et al, Gene Therapy 4:983-992, 1997; Chadwick, et al, Gene Therapy 4:937-942, 1997; Gokhale, et al, Gene Therapy 4:1289-1299, 1997; Gao, and Huang, Gene Therapy 2:710-722, 1995, all of which are incorporated by reference herein), using viral vectors (Monahan, et al, Gene Therapy 4:40-49, 1997; Onodera, et al, Blood 91:30-36, 1998, all of which are incorporated by reference herein), by uptake of “naked DNA”, and the like. Techniques well known in the art for the transfection of cells (see discussion above) can be used for the ex vivo administration of nucleic acid constructs. The exact formulation, route of administration and dosage can be chosen by the individual physician in view of the patient's condition. (Fingl et al., 1975, in “The Pharmacological Basis of Therapeutics”, Ch. 1 pl).

As disclosed herein, the push pull donor construct and methods described herein can be used for gene modification, gene correction, and gene disruption.

The push pull donor constructs and methods described herein can also be applied to stem cell based therapies, including but not limited to editing that results in: correction of somatic cell mutations; disruption of dominant negative alleles; disruption of genes required for the entry or productive infection of pathogens into cells; enhanced tissue engineering, for example, by editing gene activity to promote the differentiation or formation of functional tissues; and/or disrupting gene activity to promote the differentiation or formation of functional tissues; blocking or inducing differentiation, for example, by editing genes that block differentiation to promote stem cells to differentiate down a specific lineage pathway. Cell types for this procedure include but are not limited to, T-cells, B cells, hematopoietic stem cells, and embryonic stem cells. Additionally, induced pluripotent stem cells (iPSC) may be used which would also be generated from a patient's own somatic cells. Therefore, these stem cells or their derivatives (differentiated cell types or tissues) could be potentially engrafted into any person regardless of their origin or histocompatibility.

In some embodiments, the methods and compositions of the invention are used to supply a transgene encoding one or more therapeutics in a hematopoietic stem cell such that mature cells (e.g., RBCs) derived from these cells contain the therapeutic. These stem cells can be differentiated in vitro or in vivo and may be derived from a universal donor type of cell which can be used for all subjects. Additionally, the cells may contain a transmembrane protein to traffic the cells in the body. Treatment can also comprise use of subject cells containing the therapeutic transgene where the cells are developed ex vivo and then introduced back into the subject. For example, HSC containing a suitable transgene may be inserted into a subject via an autologous bone marrow transplant. Alternatively, stem cells such as muscle stem cells or iPSC which have been edited using with the transgene maybe also injected into muscle tissue.

Thus, this technology may be of use in a condition where a subject is deficient in some protein due to problems (e.g., problems in expression level or problems with the protein expressed as sub- or non-functioning

By way of non-limiting examples, production of the defective or missing proteins is accomplished and used to treat diseases and disorders. Nucleic acid donors encoding the proteins may be inserted into a safe harbor locus (e.g. albumin) and expressed either using an exogenous promoter or using the promoter present at the safe harbor. Alternatively, donors can be used to correct the defective gene in situ. The desired transgene may be inserted into a CD34+ stem cell and returned to a subject during a bone marrow transplant. Finally, the nucleic acid donor maybe be inserted into a CD34+ stem cell at a beta globin locus such that the mature red blood cell derived from this cell has a high concentration of the biologic encoded by the nucleic acid donor. The biologic-containing RBC can then be targeted to the correct tissue via transmembrane proteins (e.g. receptor or antibody). Additionally, the RBCs may be sensitized ex vivo via electrosensitization to make them more susceptible to disruption following exposure to an energy source (see International Patent Publication No. WO 2002/007752).

In addition to therapeutic applications, the push-pull donor polynucleotide construct and methods described herein can be used for cell line engineering and the construction of disease models.

In one aspect, provided herein is a push-pull donor polynucleotide construct as disclosed herein, for use in treating a disease or disorder.

In one aspect, provided herein is a vector as disclosed herein, for use in treating a disease or disorder.

In one aspect, provided herein is a pharmaceutical composition as disclosed herein, for use in treating a disease or disorder.

In one aspect provided herein is a push-pull donor polynucleotide construct as disclosed herein, for use in modifying the genome of a cell.

In one aspect, provided herein is a vector as disclosed herein, for use in modifying the genome of a cell.

In one aspect, provided herein is a pharmaceutical composition as disclosed herein, for use in modifying the genome of a cell.

In one aspect provided herein is a push-pull donor polynucleotide construct as disclosed herein, for use in correcting a disease-causing mutation in the genome of a cell.

In one aspect, provided herein is a vector as disclosed herein, for use in correcting a disease-causing mutation in the genome of a cell.

In one aspect, provided herein is a pharmaceutical composition as disclosed herein, for use in correcting a disease-causing mutation in the genome of a cell.

In one aspect provided herein is a push-pull donor polynucleotide construct as disclosed herein, for use in integrating an exogenous nucleotide sequence or transgene into a target nucleotide sequence in a gene of a cell.

In one aspect, provided herein is a vector as disclosed herein, for use in integrating an exogenous nucleotide sequence or transgene into a target nucleotide sequence in a gene of a cell.

In one aspect, provided herein is a pharmaceutical composition as disclosed herein, for use in integrating an exogenous nucleotide sequence or transgene into a target nucleotide sequence in a gene of a cell.

In one aspect provided herein is a push-pull donor polynucleotide construct as disclosed herein, for use in disrupting a target nucleotide sequence in a gene of a cell, wherein said gene comprises a mutation associated with a disease or disorder.

In one aspect provided herein is a vector as disclosed herein, for use in disrupting a target nucleotide sequence in a gene of a cell, wherein said gene comprises a mutation associated with a disease or disorder.

In one aspect provided herein is a pharmaceutical composition as disclosed herein, for use in disrupting a target nucleotide sequence in a gene of a cell, wherein said gene comprises a mutation associated with a disease or disorder.

In one aspect provided herein is a push-pull donor polynucleotide construct as disclosed herein, for use in modifying a target nucleotide sequence in the genome of a cell.

In one aspect, provided herein is a vector as disclosed herein, for use in modifying a target nucleotide sequence in the genome of a cell.

In one aspect, provided herein is a pharmaceutical composition as disclosed herein, for use in modifying a target nucleotide sequence in the genome of a cell.

In one aspect, provided herein is a push-pull donor polynucleotide construct as disclosed herein, for use in treating a disease or disorder.

In one aspect, provided herein is a vector as disclosed herein, for use in treating a disease or disorder.

In one aspect, provided herein is a pharmaceutical composition as disclosed herein, for use in treating a disease or disorder.

In one aspect provided herein is a push-pull donor polynucleotide construct as disclosed herein, for use in modifying the genome of a cell.

In one aspect, provided herein is a vector as disclosed herein, for use in modifying the genome of a cell.

In one aspect, provided herein is a pharmaceutical composition as disclosed herein, for use in modifying the genome of a cell.

In one aspect provided herein is a push-pull donor polynucleotide construct as disclosed herein, for use in correcting a disease-causing mutation in the genome of a cell.

In one aspect, provided herein is a vector as disclosed herein, for use in correcting a disease-causing mutation in the genome of a cell.

In one aspect, provided herein is a pharmaceutical composition as disclosed herein, for use in correcting a disease-causing mutation in the genome of a cell.

In one aspect provided herein is a push-pull donor polynucleotide construct as disclosed herein, for use in integrating an exogenous nucleotide sequence or transgene into a target nucleotide sequence in a gene of a cell.

In one aspect, provided herein is a vector as disclosed herein, for use in integrating an exogenous nucleotide sequence or transgene into a target nucleotide sequence in a gene of a cell.

In one aspect, provided herein is a pharmaceutical composition as disclosed herein, for use in integrating an exogenous nucleotide sequence or transgene into a target nucleotide sequence in a gene of a cell.

In one aspect provided herein is a push-pull donor polynucleotide construct as disclosed herein, for use in disrupting a target nucleotide sequence in a gene of a cell, wherein said gene comprises a mutation associated with a disease or disorder.

In one aspect provided herein is a vector as disclosed herein, for use in disrupting a target nucleotide sequence in a gene of a cell, wherein said gene comprises a mutation associated with a disease or disorder.

In one aspect provided herein is a pharmaceutical composition as disclosed herein, for use in disrupting a target nucleotide sequence in a gene of a cell, wherein said gene comprises a mutation associated with a disease or disorder.

In one aspect provided herein is a push-pull donor polynucleotide construct as disclosed herein, for use in modifying a target nucleotide sequence in the genome of a cell.

In one aspect, provided herein is a vector as disclosed herein, for use in modifying a target nucleotide sequence in the genome of a cell.

In one aspect, provided herein is a pharmaceutical composition as disclosed herein, for use in modifying a target nucleotide sequence in the genome of a cell.

In one aspect, provided herein is a push-pull donor polynucleotide construct, a first polynucleotide encoding a first zinc finger nuclease, and a second polynucleotide encoding a second zinc finger nuclease as disclosed herein, for use in treating a disease or disorder.

In one aspect, provided herein is a push-pull donor polynucleotide construct and a polynucleotide encoding one or more zinc finger nucleases as disclosed herein, for use in treating a disease or disorder.

In one aspect, provided herein is a push-pull donor polynucleotide construct and a polynucleotide encoding a 2-in-1 zinc finger nuclease as disclosed herein, for use in treating a disease or disorder.

In one aspect provided herein is a push-pull donor polynucleotide construct, a first polynucleotide encoding a first zinc finger nuclease, and a second polynucleotide encoding a second zinc finger nuclease as disclosed herein, for use in modifying the genome of a cell.

In one aspect provided herein is a push-pull donor polynucleotide construct and a polynucleotide encoding one or more zinc finger nucleases as disclosed herein, for use in modifying the genome of a cell.

In one aspect provided herein is a push-pull donor polynucleotide construct and a polynucleotide encoding a 2-in-1 zinc finger nuclease as disclosed herein, for use in modifying the genome of a cell.

In one aspect provided herein is a push-pull donor polynucleotide construct, a first polynucleotide encoding a first zinc finger nuclease, and a second polynucleotide encoding a second zinc finger nuclease as disclosed herein, for use in correcting a disease-causing mutation in the genome of a cell.

In one aspect provided herein is a push-pull donor polynucleotide construct and a polynucleotide encoding one or more zinc finger nucleases as disclosed herein, for use in correcting a disease-causing mutation in the genome of a cell.

In one aspect provided herein is a push-pull donor polynucleotide construct and a polynucleotide encoding a 2-in-1 zinc finger nuclease as disclosed herein, for use in correcting a disease-causing mutation in the genome of a cell.

In one aspect provided herein is a push-pull donor polynucleotide construct, a first polynucleotide encoding a first zinc finger nuclease, and a second polynucleotide encoding a second zinc finger nuclease as disclosed herein, for use in integrating an exogenous nucleotide sequence or transgene into a target nucleotide sequence in a gene of a cell.

In one aspect provided herein is a push-pull donor polynucleotide construct and a polynucleotide encoding one or more zinc finger nucleases as disclosed herein, for use in integrating an exogenous nucleotide sequence or transgene into a target nucleotide sequence in a gene of a cell.

In one aspect provided herein is a push-pull donor polynucleotide construct and a polynucleotide encoding a 2-in-1 zinc finger nuclease as disclosed herein, for use in integrating an exogenous nucleotide sequence or transgene into a target nucleotide sequence in a gene of a cell.

In one aspect provided herein is a push-pull donor polynucleotide construct, a first polynucleotide encoding a first zinc finger nuclease, and a second polynucleotide encoding a second zinc finger nuclease as disclosed herein, for use in disrupting a target nucleotide sequence in a gene of a cell, wherein said gene comprises a mutation associated with a disease or disorder.

In one aspect provided herein is a push-pull donor polynucleotide construct and a polynucleotide encoding one or more zinc finger nucleases as disclosed herein, for use in disrupting a target nucleotide sequence in a gene of a cell, wherein said gene comprises a mutation associated with a disease or disorder.

In one aspect provided herein is a push-pull donor polynucleotide construct and a polynucleotide encoding a 2-in-1 zinc finger nuclease as disclosed herein, for use in disrupting a target nucleotide sequence in a gene of a cell, wherein said gene comprises a mutation associated with a disease or disorder.

In one aspect provided herein is a push-pull donor polynucleotide construct, a first polynucleotide encoding a first zinc finger nuclease, and a second polynucleotide encoding a second zinc finger nuclease as disclosed herein, for use in modifying a target nucleotide sequence in the genome of a cell.

In one aspect provided herein is a push-pull donor polynucleotide construct and a polynucleotide encoding one or more zinc finger nucleases as disclosed herein, for use in modifying a target nucleotide sequence in the genome of a cell.

In one aspect provided herein is a push-pull donor polynucleotide construct and a polynucleotide encoding a 2-in-1 zinc finger nuclease as disclosed herein, for use in modifying a target nucleotide sequence in the genome of a cell.

In one aspect, provided herein is a vector comprising a push-pull donor polynucleotide construct, a first vector comprising a first polynucleotide encoding a first zinc finger nuclease and a second vector comprising a second polynucleotide encoding a second zinc finger nuclease encoding a second zinc finger nuclease as disclosed herein, for use in treating a disease or disorder.

In one aspect, provided herein is a vector comprising a push-pull donor polynucleotide construct and a vector encoding one or more zinc finger nucleases as disclosed herein, for use in treating a disease or disorder.

In one aspect, provided herein is a vector comprising a push-pull donor polynucleotide construct, a first vector comprising a first polynucleotide encoding a first zinc finger nuclease and a second vector comprising a second polynucleotide encoding a second zinc finger nuclease as disclosed herein, for use in modifying the genome of a cell.

In one aspect, provided herein is a vector comprising a push-pull donor polynucleotide construct and a vector encoding one or more zinc finger nucleases as disclosed herein, for use in modifying the genome of a cell.

In one aspect, provided herein is a vector comprising a push-pull donor polynucleotide construct, a first vector comprising a first polynucleotide encoding a first zinc finger nuclease and a second vector encoding a second zinc finger nuclease comprising a second polynucleotide as disclosed herein, for use in correcting a disease-causing mutation in the genome of a cell.

In one aspect, provided herein is a vector comprising a push-pull donor polynucleotide construct and a vector encoding one or more zinc finger nucleases as disclosed herein, for use in correcting a disease-causing mutation in the genome of a cell.

In one aspect, provided herein is a vector comprising a push-pull donor polynucleotide construct, a first vector comprising a first polynucleotide encoding a first zinc finger nuclease and a second vector comprising a second polynucleotide encoding a second zinc finger nuclease as disclosed herein, for use in integrating an exogenous nucleotide sequence or transgene into a target nucleotide sequence in a gene of a cell.

In one aspect, provided herein is a vector comprising a push-pull donor polynucleotide construct and a vector encoding one or more zinc finger nucleases as disclosed herein, for use in integrating an exogenous nucleotide sequence or transgene into a target nucleotide sequence in a gene of a cell.

In one aspect provided herein is a vector comprising a push-pull donor polynucleotide construct, a first vector comprising a first polynucleotide encoding a first zinc finger nuclease and a second vector comprising a second polynucleotide encoding a second zinc finger nuclease as disclosed herein, for use in disrupting a target nucleotide sequence in a gene of a cell, wherein said gene comprises a mutation associated with a disease or disorder.

In one aspect provided herein is a vector comprising a push-pull donor polynucleotide construct and a vector encoding one or more zinc finger nucleases as disclosed herein, for use in disrupting a target nucleotide sequence in a gene of a cell, wherein said gene comprises a mutation associated with a disease or disorder.

In one aspect, provided herein is a vector comprising a push-pull donor polynucleotide construct, a first vector comprising a first polynucleotide encoding a first zinc finger nuclease and a second vector comprising a second polynucleotide encoding a second zinc finger nuclease as disclosed herein, for use in modifying a target nucleotide sequence in the genome of a cell.

In one aspect, provided herein is a vector comprising a push-pull donor polynucleotide construct and a vector encoding one or more zinc finger nucleases as disclosed herein, for use in modifying a target nucleotide sequence in the genome of a cell.

The methods and compositions disclosed herein can be used in any type of cell including a eukaryotic or prokaryotic cell and/or cell line. Examples of cells include, but are not limited to, prokaryotic cells, fungal cells, Archaeal cells, plant cells, insect cells, animal cells, vertebrate cells, mammalian cells and human cells. In some embodiments, the cell is a eukaryotic cell. In some embodiments, the cell is a mammalian cell. In some embodiments, the mammalian cell is a stem cell. In some embodiments, the eukaryotic cell is a human cell.

In some embodiments, the eukaryotic cell is a plant cell. In some embodiments, the cell is a non-dividing cell. In some embodiments, the eukaryotic cell is a non-dividing cell. In some embodiments, the mammalian cell is a non-dividing cell. In some embodiments, the stem cell is a non-dividing cell. In some embodiments, the human cell is a non-dividing cell. In some embodiments, the cell is a hepatocyte. In some embodiments, the eukaryotic cell is a hepatocyte. In some embodiments, the mammalian cell is a hepatocyte. In some embodiments, the stem cell is a hepatocyte. In some embodiments, the human cell is a hepatocyte. Non-limiting examples of eukaryotic cells or cell lines generated from such cells include T-cells, COS, K562, CHO (e.g., CHO-S, CHO-K1, CHO-DG44, CHO-DUXB11, CHO-DUKX, CHOK1SV), VERO, MDCK, W138, V79, B14AF28-G3, BHK, HaK, NSO, SP2/0-Ag14, HeLa, HEK293 (e.g., HEK293-F, HEK293-H, HEK293-T), perC6, HepG2, and 348A cells, as well as, insect cells such as Spodoptera frugiperda (Sf), or fungal cells such as Saccharomyces, Pichia and Schizosaccharomyces. Examples of stem cells include, but are not limited to, embryonic stem cells, induced pluripotent stem cells (iPS cells), hematopoietic stem cells, neuronal stem cells and mesenchymal stem cells.

In some embodiments, in order to introduce the push-pull donor polynucleotide construct into the cell, the nucleic acid sequence of the push-pull donor polynucleotide construct is incorporated into a plasmid, a viral vector, a mini-circle, a linear DNA form or other delivery system. Such delivery systems are well known to those of skill in the art.

In some embodiments, the target nucleotide sequence is an endogenous locus. In some embodiments, the endogenous locus is selected from the group consisting of Iduronidase Alpha-L (IDUA) gene (associated with mucopolysaccharidosis type I (MPS I)), Iduronate 2-Sulfatase (IDS) gene (associated with mucopolysaccharidosis type II (MPS II)), alpha-Galactosidase (GLA) gene (associated with Fabry disease), alpha-Glucosidase (GAA) gene (associated with Pompe disease), Phenylalanine Hydroxylase (PAH) gene (associated with phenylketonuria (PKU)), and a safe-harbor locus. In some embodiments, the endogenous locus is selected from the group consisting of alpha-D-mannosidase (MAN2B1) gene (associated with alpha-mannosidosis), N-aspartyl-beta-glucosaminidase (AGA) gene (associated with Aspartylglucosaminuria), lysosomal acid lipase (LIPA) gene (associated with cholesteryl ester storage disease, lysosomal acid lipase deficiency and Wolman disease), cystinosin (CTNS) gene (associated with cystinosis), lysosomal associated membrane 2 (LAMP2) gene (associated with Danon disease), acid ceramidase (ASAH1) gene (associated with Farber disease), alpha fucosidase (FUCA1) gene (associated with fucosidosis), Cathepsin A (CTSA) gene (associated with Galactosialidosis), acid beta-glucocerebrosidase (GBA) gene (associated with Gaucher Disease Types I, II and III), beta galactosidase (GLB1) gene (associated with GM1 Gangliosidosis Types I, II and III or MPS IV—Morquio Type B), beta hexosaminidase A and B (HEXB) gene (associated with GM2 Sandhoff Disease I/J/A), beta-hexosaminidase (HEXA) gene (associated with GM2 Tay-Sachs disease), GM2 ganglioside activator (GM2A) gene (associated with GM2 Gangliosidosis AB variant), GLcNAc-1-phosphotransferase (GNPTAB) gene (associated with I-Cell Disease/Mucolipidosis II), Beta-galactosylceramidase (GALC) gene (associated with Krabbe disease), arylsulfatase A (ARSA) gene (associated with metachromatic leukodystrophy), heparan-N-sulfatase (SGSH) gene (associated with MPS IIIA—Sanfilippo Syndrome Type A), alpha-N-acetylglucosaminidase (NAGLU) gene (associated with MPS IIIB—Sanfilippo Syndrome Type B), acetyle coA:alpha-flucosaminide acetyltransferase (GSNAT) gene (associated with MPS IIIC—Sanfilippo Syndrome Type C), N-acetyl glucosamine-6-sulfatase (GALNS) gene (associated with MPS IV—Morquio Type A), arylsulfatase B (ARSB) gene (associated with MPS VI—Maroteaux-Lamy), beta-glucuronidase (GUSB) gene (associated with MPS VII-Sly Syndrome), Hyaluronidase (HYAL1) gene (MPS IX—Hyaluronidase Deficiency), Neuraminidase (NEU1) gene (associated with Mucolipidosis I—Sialidosis), GlcNAc-1-phosphotransferase (GNPTG) gene (associated with Mucolipidosis IIIC), mucolipin-1 (MCOLN1) gene (associated with Mucolipidosis Type IV), formylglycine-generating enzyme (SUMF1) gene (associated with Multiple Sulfatase Deficiency), palmitoyl-protein thioesterase 1 (PPT1) gene (associated with Neuronal Ceroid Lipofuscinosis T1), tripeptidyl peptidase 1 (TPP1) gene (associated with Neuronal Ceroid Lipofuscinosis T2), CLN3 (CLN3) gene (associated with Neuronal Ceroid Lipofuscinosis T3), Cysteine string protein alpha (DNAJC5) gene (associated with Neuronal Ceroid Lipofuscinosis T4), CLN5 (CLN5) gene (associated with Neuronal Ceroid Lipofuscinosis T5), CLN6 (CLN6) gene (associated with Neuronal Ceroid Lipofuscinosis T6), CLN7 (CLN7) gene (associated with Neuronal Ceroid Lipofuscinosis T7), CLN8 (CLN8) gene (associated with Neuronal Ceroid Lipofuscinosis T8), acid sphingomyelinase (SMPD1) gene (associated with Niemann-Pick Disease Type A and B), NPC1 and NPC2 (NP1 and NPC2) genes (associated with Niemann-Pick Disease Type C), cathepsin K (CTSK) gene (associated with pycnodysostosis), sialin (SLC17A5) gene (associated with sialic acid storage disease), alpha-N-acetylgalactosaminidase (NAGA) gene (associated with Schindler disease), glucose-6-phosphatase (G6PC) gene (associated with GSD1a), solute carrier family 37 member 4 (SLC37A4) gene (associated with GSD1a), argininosuccinate synthase 1 (ASS1) gene (associated with Citrullinemia), solute carrier family 25 member 13 (SLC25A13) gene (associated with Citrullinemia), ornithine transcarbamylase (OTC) gene (associated with OTC deficiency), and a safe-harbor locus.

In some embodiments, the endogenous locus is selected from FGFR3 gene (associated with achondroplasia), CNGA3/CNGB3/GNAT2/PDE6C/PDE6H genes (associated with achromatopsia), GAA gene (associated with Pompe disease or acid maltase deficiency), ADA gene (associated with adenosine deaminase deficiency (OMIM No. 102700)), ABCD1 gene (associated with X-linked adrenoleukodystrophy), X chromosome (associated with aicardi syndrome), SERPINA1 gene (associated with alpha-1 antitrypsin deficiency), HBA1 and HBA2 genes (associated with alpha-thalassemia), AR gene (associated with androgen insensitivity syndrome), FGFR2 gene (associated with apert syndrome), PKP2 (associated with arrhythmogenic right ventricular), SLC26A2 (associated with diastrophic dysplasia), ATM gene (associated with ataxia telangiectasia), TAZ gene (associated with barth syndrome), HBB gene (associated with beta-thalassemia or sickle cell disease (sickle cell anemia)), ASPA gene (associated with canavan disease), CYBA/CYBB/NCF1/NCF2/NCF4 genes (associated with chronic granulomatous diseases), short (p) arm of chromosome 5 (deletion associated with cri-du-chat syndrome), CTFR gene (associated with cystic fibrosis), EDA/EDAR/EDARADD/WNT10A genes (associated with hypohidrotic ectodermal dysplasia), GLA gene (associated with Fabry disease), FANCA/FANCC/FANCG genes (associated with fanconi anemia), ACVR1 gene (associated with fibrodysplasia ossificans progressive), FMR1 gene (associated with fragile X syndrome), GALT/GALK1/GALE genes (associated with galactosemia), GBA gene (associated with Gaucher's disease), GLB1 gene (associated with generalized gangliosidoses (e.g., GM1)), HFE gene (associated with Type 1 hemochromatosis), HJV and HAMP genes (associated with Type 2 hemochromatosis), TFR2 gene (associated with Type 3 hemochromatosis), SLC40A1 gene (associated with Type 4 hemochromatosis), HBB gene (associated with hemoglobin C mutation in the 6th codon of beta-globin (HbC), hemophilia), IDS gene (associated with Hunter syndrome also known as mucopolysaccharidosis type II (MPS II)), HTT gene (associated with Huntington's disease), IDUA gene (associated with Hurler Syndrome also known as MPS I), ALPL gene (associated with hypophosphatasia), X chromosome (extra chromosome associated with Klinefelter syndrome), GALC gene (associated with Krabbes Disease), long (q) arm of chromosome 8 (deletion associated with Langer-Giedion Syndrome also known as TRPS II), ITGB2 gene (associated with leukocyte adhesion deficiency (LAD, OMIM No. 116920)), ARSA gene (associated with metachromatic leukodystrophy), CACNA1C gene (associated with long QT syndrome), LPL gene (associated with lipoprotein lipase deficiency), FBN1 gene (associated with Marfan syndrome), chromosome 3, 10 or 13 (associated with Moebius syndrome), GNS/HGSNAT/NAGLU/SGSH genes (associated with Sanfilippo syndrome also known as MPS III), GALNS and GLB1 (associated with MPS IV), ARSB gene (associated with MPS VI), GUSB gene (associated with MPS VII), LMX1B gene (associated with nail patella syndrome), AVPR2 and AQP2 genes (associated with nephrogenic diabetes insipidus), NF1 gene (associated with neurofibromatosis type 1), NF2 gene (associated with neurofibromatosis type 2), SMPD1 gene (associated with Niemann-Pick disease Type A and B), NPC1 or NPC2 genes (associated with Niemann-Pick disease Type C), COL1A1 and COL1A2 genes (associated with osteogenesis imperfecta), PAH gene (associated with phenylketonuria (PKU)), ALAD/ALAS2/CPOX/FECH/HMBS/PPOX/UROD/UROS genes (associated with porphyria), OCA2 or chromosome 15 (deletion associated with Prader-Willi syndrome), LMNA gene (associated with Hutchinson-Gilford progeria syndrome), AKT1 gene (associated with Proteus syndrome), RB1 gene (associated with retinoblastoma), MECP2 gene (associated with Rett syndrome), CREBBP gene (associated with Rubinstein-Taybi syndrome), IL2RG gene (associated with severe combined immunodeficiency (SCID)), SBDS gene (associated with Shwachman-Diamond syndrome), chromosome 17 (small deletion associated with Smith-Magenis syndrome), COL2A1 and COL11A1 genes (associated with Stickler syndrome), HEXA gene (associated with Tay-Sachs disease), RBM8A gene (associated with Thrombocytopenia Absent Radius (TAR) syndrome), TCOF1/POLR1C/POLR1D genes (associated with Treacher Collins syndrome), chromosome 13 (associated with trisomy 13), chromosome 18 (associated with trisomy 18), TSC1 or TSC2 genes (associated with tuberous sclerosis), X chromosome (monosomy associated with Turner syndrome), ASL gene (associated with urea cycle disorder), VHL gene (associated with von Hippel-Landau disease), EDN3/EDNRB/MITF/PAX3/SNAI2/SOX10 genes (associated with Waardenburg syndrome), chromosome 7: CLIP2/ELN/GTF2I/GTF2IRD1/LIMK1/NCF1 genes (deletions associated with Williams syndrome), ATP7B gene (associated with Wilson disease), WAS gene (associated with Wiskott-Aldrich syndrome), and SH2D1A and XIAP genes (associated with X-linked lymphoproliferative syndrome (XLP, OMIM No. 308240)), PEX1/10/26 (associated with Zellweger spectrum disorder), and a safe harbor locus.

In some embodiments of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In some embodiments, the donor sequence can contain sequences that are homologous, but not identical, to genomic sequences in the region of interest, thereby stimulating homologous recombination to insert a non-identical sequence in the region of interest. In some embodiments, portions of the donor sequence that are homologous to sequences in the region of interest exhibit between about 80 to 99% (or any integer therebetween) sequence identity to the genomic sequence that is replaced. In some embodiments, the homology between the donor and genomic sequence is higher than 99%, for example if only 1 nucleotide differs as between donor and genomic sequences of over 100 contiguous base pairs. In some embodiments, a non-homologous portion of the donor sequence contains sequences that are not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In some embodiments, the donor sequence is non-homologous to the first target sequence, and is inserted into the genome by non-homologous recombination mechanisms.

In some embodiments, the disclosure provides for the integration of an exogenous nucleic acid sequence into a safe harbor locus in the genome of a cell. A safe harbor locus is typically a genomic locus where transgenes can integrate and function in a predictable manner without perturbing endogenous gene activity. Exemplary safe harbor loci in the human genome include, without limitation the Rosa26 locus, the AAVS 1 locus, and the safe harbor loci listed in Sadelain et al. Nat Rev Cancer. 2012; 12(1):51-8. In some embodiments, the safe harbor locus is located in chromosome 1.

The polynucleotide constructs, vectors and pharmaceutical compositions disclosed herein may be delivered to isolated cells (which in turn may be administered to a living subject for ex vivo cell therapy) or to a living subject. Delivery of gene editing molecules to cells and subjects are known in the art. Methods of delivering zinc finger nuclease proteins as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Suitable cells include, but are not limited to, eukaryotic and prokaryotic cells and/or cell lines. Non-limiting examples of eukaryotic cells or cell lines generated from such cells include T-cells, COS, K562, CHO (e.g., CHO-S, CHO-K1, CHO-DG44, CHO-DUXB11, CHO-DUKX, CHOK1SV), VERO, MDCK, W138, V79, B14AF28-G3, BHK, HaK, NSO, SP2/0-Ag14, HeLa, HEK293 (e.g., HEK293-F, HEK293-H, HEK293-T), perC6, HepG2 and 348A cells, as well as, insect cells such as Spodoptera frugiperda (Sf), or fungal cells such as Saccharomyces, Pichia and Schizosaccharomyces. In some embodiments, the cell is a mammalian cell. In some embodiments, the cell is a stem cell, such as, by way of example, embryonic stem cells, induced pluripotent stem cells (iPS cells), hematopoietic stem cells, neuronal stem cells and mesenchymal stem cells.

In some embodiments, push-pull donor polynucleotide constructs may be delivered via vectors. The nucleic acid encoding the one or more zinc finger nuclease variant protein, as described herein, may also be delivered using vectors containing sequences encoding one or more of the components of the zinc finger nuclease protein. Furthermore, it will be apparent that any of these vectors may comprise one or more DNA-binding protein-encoding sequences and/or additional nucleic acids as appropriate. Thus, when one or more zinc finger nuclease protein as described herein are introduced into the cell, and additional DNAs as appropriate, they may be carried on the same vector or on different vectors. When multiple vectors are used, each vector may comprise a sequence encoding one or multiple zinc finger nuclease proteins and additional nucleic acids as desired. Conventional viral and non-viral based gene transfer methods can be used to introduce nucleic acids encoding engineered DNA-binding proteins in cells (e.g., in mammalian cells) and target tissues and to co-introduce additional nucleotide sequences as desired. Such methods can also be used to administer nucleic acids to cells in vitro. In certain embodiments, nucleic acids are administered for in vivo or ex vivo gene therapy uses.

Gene therapy vectors comprising the push-pull donor polynucleotide constructs or nucleic acid encoding the zinc finger nuclease of the disclosure can be delivered in vivo by administration to an individual patient (subject), typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by re-implantation of the cells into a patient, usually after selection for cells which have incorporated the vector.

Ex vivo cell transfection for diagnostics, research, transplant or for gene therapy (e.g., via re-infusion of the transfected cells into the host organism) is well known to those of skill in the art. In some embodiments, cells are isolated from the subject organism, transfected with a push-pull donor polynucleotide construct and/or nucleic acid encoding zinc finger nucleases, and re-infused back into the subject organism (e.g., patient). Various cell types suitable for ex vivo transfection are well-known to those of skill in the art (see, e.g., Freshney, et al., Culture of Animal Cells, A Manual of Basic Technique (3rd ed. 1994)) and the references cited therein for a discussion of how to isolate and culture cells from patients).

In some embodiments, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba, et al. (1992) J. Exp. Med. 176:1693-1702).

In another aspect, provided herein is the use of any of the push-pull donor polynucleotide constructs disclosed herein, for the preparation of a medicament for treating a disease or disorder.

In another aspect, provided herein is the use of any of the push-pull donor polynucleotide constructs, a first polynucleotide encoding a first zinc finger nuclease, and a second polynucleotide encoding a second zinc finger nuclease disclosed herein, for the preparation of a medicament for treating a disease or disorder.

In another aspect, provided herein is the use of any of the push-pull donor polynucleotide constructs and a polynucleotide encoding one or more zinc finger nucleases disclosed herein, for the preparation of a medicament for treating a disease or disorder.

In another aspect, provided herein is the use of any of the push-pull donor polynucleotide constructs and a polynucleotide encoding a 2-in-1 zinc finger nuclease disclosed herein, for the preparation of a medicament for treating a disease or disorder.

In another aspect, provided herein is the use of any of the push-pull donor polynucleotide constructs disclosed herein, for the preparation of a medicament for modifying the genome of a cell.

In another aspect, provided herein is the use of any of the push-pull donor polynucleotide constructs, a first polynucleotide encoding a first zinc finger nuclease, and a second polynucleotide encoding a second zinc finger nuclease disclosed herein, for the preparation of a medicament for modifying the genome of a cell.

In another aspect, provided herein is the use of any of the push-pull donor polynucleotide constructs and a polynucleotide encoding one or more zinc finger nucleases disclosed herein, for the preparation of a medicament for modifying the genome of a cell.

In another aspect, provided herein is the use of any of the push-pull donor polynucleotide constructs and a polynucleotide encoding a 2-in-1 zinc finger nuclease disclosed herein, for the preparation of a medicament for modifying the genome of a cell.

In another aspect, provided herein is the use of any of the push-pull donor polynucleotide constructs disclosed herein, for the preparation of a medicament for integrating a transgene into a target nucleotide sequence of a cell.

In another aspect, provided herein is the use of any of the push-pull donor polynucleotide constructs, a first polynucleotide encoding a first zinc finger nuclease, and a second polynucleotide encoding a second zinc finger nuclease disclosed herein, for the preparation of a medicament for integrating a transgene into a target nucleotide sequence of a cell.

In another aspect, provided herein is the use of any of the push-pull donor polynucleotide constructs and a polynucleotide encoding one or more zinc finger nucleases disclosed herein, for the preparation of a medicament for integrating a transgene into a target nucleotide sequence of a cell.

In another aspect, provided herein is the use of any of the push-pull donor polynucleotide constructs and a polynucleotide encoding a 2-in-1 zinc finger nuclease disclosed herein, for the preparation of a medicament for integrating a transgene into a target nucleotide sequence of a cell.

In another aspect, provided herein is the use of any of the push-pull donor polynucleotide constructs disclosed herein, for the preparation of a medicament for disrupting a target nucleotide sequence in a cell.

In another aspect, provided herein is the use of any of the push-pull donor polynucleotide constructs, a first polynucleotide encoding a first zinc finger nuclease, and a second polynucleotide encoding a second zinc finger nuclease disclosed herein, for the preparation of a medicament for disrupting a target nucleotide sequence in a cell.

In another aspect, provided herein is the use of any of the push-pull donor polynucleotide constructs and a polynucleotide encoding one or more zinc finger nucleases disclosed herein, for the preparation of a medicament for disrupting a target nucleotide sequence in a cell.

In another aspect, provided herein is the use of any of the push-pull donor polynucleotide constructs and a polynucleotide encoding a 2-in-1 zinc finger nuclease disclosed herein, for the preparation of a medicament for disrupting a target nucleotide sequence in a cell.

In another aspect, provided herein is the use of any of the push-pull donor polynucleotide constructs disclosed herein, for the preparation of a medicament for correcting a disease-causing mutation in the genome of a cell.

In another aspect, provided herein is the use of any of the push-pull donor polynucleotide constructs, a first polynucleotide encoding a first zinc finger nuclease, and a second polynucleotide encoding a second zinc finger nuclease disclosed herein, for the preparation of a medicament for correcting a disease-causing mutation in the genome of a cell.

In another aspect, provided herein is the use of any of the push-pull donor polynucleotide constructs and a polynucleotide encoding one or more zinc finger nucleases disclosed herein, for the preparation of a medicament for correcting a disease-causing mutation in the genome of a cell.

In another aspect, provided herein is the use of any of the push-pull donor polynucleotide constructs and a polynucleotide encoding a 2-in-1 zinc finger nuclease disclosed herein, for the preparation of a medicament for correcting a disease-causing mutation in the genome of a cell.

In another aspect, provided herein is the use of any of the push-pull donor polynucleotide constructs disclosed herein, for the preparation of a medicament for modifying a target nucleotide sequence in the genome of a cell.

In another aspect, provided herein is the use of any of the push-pull donor polynucleotide constructs, a first polynucleotide encoding a first zinc finger nuclease, and a second polynucleotide encoding a second zinc finger nuclease disclosed herein, for the preparation of a medicament for modifying a target nucleotide sequence in the genome of a cell.

In another aspect, provided herein is the use of any of the push-pull donor polynucleotide constructs and a polynucleotide encoding one or more zinc finger nucleases disclosed herein, for the preparation of a medicament for modifying a target nucleotide sequence in the genome of a cell.

In another aspect, provided herein is the use of any of the push-pull donor polynucleotide constructs and a polynucleotide encoding a 2-in-1 zinc finger nuclease disclosed herein, for the preparation of a medicament for modifying a target nucleotide sequence in the genome of a cell.

In another aspect, provided herein is the use of any of the vectors disclosed herein, for the preparation of a medicament for treating a disease or disorder.

In another aspect, provided herein is the use of any of the vectors comprising a push-pull donor polynucleotide construct disclosed herein, for the preparation of a medicament for treating a disease or disorder.

In another aspect, provided herein is the use of any of the vectors comprising a push-pull donor polynucleotide construct, a vector comprising a first polynucleotide encoding a first zinc finger nuclease, and a vector comprising a second polynucleotide encoding a second zinc finger nuclease disclosed herein, for the preparation of a medicament for treating a disease or disorder.

In another aspect, provided herein is the use of any of the vectors comprising a push-pull donor polynucleotide construct and a vector comprising a polynucleotide encoding one or more zinc finger nucleases disclosed herein, for the preparation of a medicament for treating a disease or disorder.

In another aspect, provided herein is the use of any of the vectors comprising a push-pull donor polynucleotide construct and a vector comprising a polynucleotide encoding a 2-in-1 zinc finger nuclease disclosed herein, for the preparation of a medicament for treating a disease or disorder.

In another aspect, provided herein is the use of any of the vectors disclosed herein, for the preparation of a medicament for modifying the genome of a cell.

In another aspect, provided herein is the use of any of the vectors comprising a push-pull donor polynucleotide construct disclosed herein, for the preparation of a medicament for modifying the genome of a cell.

In another aspect, provided herein is the use of any of the vectors comprising a push-pull donor polynucleotide construct, a vector comprising a first polynucleotide encoding a first zinc finger nuclease, and a vector comprising a second polynucleotide encoding a second zinc finger nuclease disclosed herein, for the preparation of a medicament for modifying the genome of a cell.

In another aspect, provided herein is the use of any of the vectors comprising a push-pull donor polynucleotide construct and a vector comprising a polynucleotide encoding one or more zinc finger nucleases disclosed herein, for the preparation of a medicament for modifying the genome of a cell.

In another aspect, provided herein is the use of any of the vectors comprising a push-pull donor polynucleotide construct and a vector comprising a polynucleotide encoding a 2-in-1 zinc finger nuclease disclosed herein, for the preparation of a medicament for modifying the genome of a cell.

In another aspect, provided herein is the use of any of the vectors disclosed herein, for the preparation of a medicament for integrating a transgene into a target nucleotide sequence of a cell.

In another aspect, provided herein is the use of any of the vectors comprising a push-pull donor polynucleotide construct disclosed herein, for the preparation of a medicament for integrating a transgene into a target nucleotide sequence of a cell.

In another aspect, provided herein is the use of any of the vectors comprising a push-pull donor polynucleotide construct, a vector comprising a first polynucleotide encoding a first zinc finger nuclease, and a vector comprising a second polynucleotide encoding a second zinc finger nuclease disclosed herein, for the preparation of a medicament for integrating a transgene into a target nucleotide sequence of a cell.

In another aspect, provided herein is the use of any of the vectors comprising a push-pull donor polynucleotide construct and a vector comprising a polynucleotide encoding one or more zinc finger nucleases disclosed herein, for the preparation of a medicament for integrating a transgene into a target nucleotide sequence of a cell.

In another aspect, provided herein is the use of any of the vectors comprising a push-pull donor polynucleotide constructs and a vector comprising a polynucleotide encoding a 2-in-1 zinc finger nuclease disclosed herein, for the preparation of a medicament for integrating a transgene into a target nucleotide sequence of a cell.

In another aspect, provided herein is the use of any of the vectors disclosed herein, for the preparation of a medicament for disrupting a target nucleotide sequence in a cell.

In another aspect, provided herein is the use of any of the vectors comprising a push-pull donor polynucleotide construct disclosed herein, for the preparation of a medicament for disrupting a target nucleotide sequence in a cell.

In another aspect, provided herein is the use of any of the vectors comprising a push-pull donor polynucleotide construct, a vector comprising a first polynucleotide encoding a first zinc finger nuclease, and a vector comprising a second polynucleotide encoding a second zinc finger nuclease disclosed herein, for the preparation of a medicament for disrupting a target nucleotide sequence in a cell.

In another aspect, provided herein is the use of any of the vectors comprising a push-pull donor polynucleotide construct and a vector comprising a polynucleotide encoding one or more zinc finger nucleases disclosed herein, for the preparation of a medicament for disrupting a target nucleotide sequence in a cell.

In another aspect, provided herein is the use of any of the vectors comprising a push-pull donor polynucleotide construct and a vector comprising polynucleotide encoding a 2-in-1 zinc finger nuclease disclosed herein, for the preparation of a medicament for disrupting a target nucleotide sequence in a cell.

In another aspect, provided herein is the use of any of the vectors disclosed herein, for the preparation of a medicament for correcting a disease-causing mutation in the genome of a cell.

In another aspect, provided herein is the use of any of the vectors comprising a push-pull donor polynucleotide construct disclosed herein, for the preparation of a medicament for correcting a disease-causing mutation in the genome of a cell.

In another aspect, provided herein is the use of any of the vectors comprising a push-pull donor polynucleotide constructs, a vector comprising a first polynucleotide encoding a first zinc finger nuclease, and a vector comprising a second polynucleotide encoding a second zinc finger nuclease disclosed herein, for the preparation of a medicament for correcting a disease-causing mutation in the genome of a cell.

In another aspect, provided herein is the use of any of the vectors comprising a push-pull donor polynucleotide construct and a vector comprising a polynucleotide encoding one or more zinc finger nucleases disclosed herein, for the preparation of a medicament for correcting a disease-causing mutation in the genome of a cell.

In another aspect, provided herein is the use of any of the vectors comprising a push-pull donor polynucleotide construct and a vector comprising a polynucleotide encoding a 2-in-1 zinc finger nuclease disclosed herein, for the preparation of a medicament for correcting a disease-causing mutation in the genome of a cell.

In another aspect, provided herein is the use of any of the vectors disclosed herein, for the preparation of a medicament for modifying a target nucleotide sequence in the genome of a cell.

In another aspect, provided herein is the use of any of the vectors comprising the push-pull donor polynucleotide constructs disclosed herein, for the preparation of a medicament for modifying a target nucleotide sequence in the genome of a cell.

In another aspect, provided herein is the use of any of the vectors comprising a push-pull donor polynucleotide construct, a vector comprising a first polynucleotide encoding a first zinc finger nuclease, and a vector comprising a second polynucleotide encoding a second zinc finger nuclease disclosed herein, for the preparation of a medicament for modifying a target nucleotide sequence in the genome of a cell.

In another aspect, provided herein is the use of any of the vector comprising a push-pull donor polynucleotide construct and a vector comprising a polynucleotide encoding one or more zinc finger nucleases disclosed herein, for the preparation of a medicament for modifying a target nucleotide sequence in the genome of a cell.

In another aspect, provided herein is the use of any of the vectors comprising a push-pull donor polynucleotide construct and a vector comprising a polynucleotide encoding a 2-in-1 zinc finger nuclease disclosed herein, for the preparation of a medicament for modifying a target nucleotide sequence in the genome of a cell.

In another aspect, provided herein is any of the push-pull donor polynucleotide constructs disclosed herein, for use in treating a disease or disorder.

In another aspect, provided herein is any of the push-pull donor polynucleotide constructs, a first polynucleotide encoding a first zinc finger nuclease, and a second polynucleotide encoding a second zinc finger nuclease disclosed herein, for use in treating a disease or disorder.

In another aspect, provided herein is any of the push-pull donor polynucleotide constructs and a polynucleotide encoding one or more zinc finger nucleases disclosed herein, for use in treating a disease or disorder.

In another aspect, provided herein is any of the push-pull donor polynucleotide constructs and a polynucleotide encoding a 2-in-1 zinc finger nuclease disclosed herein, for use in treating a disease or disorder.

In another aspect, provided herein is any of the push-pull donor polynucleotide constructs disclosed herein, for use in modifying the genome of a cell.

In another aspect, provided herein is any of the push-pull donor polynucleotide constructs, a first polynucleotide encoding a first zinc finger nuclease, and a second polynucleotide encoding a second zinc finger nuclease disclosed herein, for use in modifying the genome of a cell.

In another aspect, provided herein is any of the push-pull donor polynucleotide constructs and a polynucleotide encoding one or more zinc finger nucleases disclosed herein, for use in modifying the genome of a cell.

In another aspect, provided herein is any of the push-pull donor polynucleotide constructs and a polynucleotide encoding a 2-in-1 zinc finger nuclease disclosed herein, for use in modifying the genome of a cell.

In another aspect, provided herein is any of the push-pull donor polynucleotide constructs disclosed herein, for use in integrating a transgene into a target nucleotide sequence of a cell.

In another aspect, provided herein is any of the push-pull donor polynucleotide constructs, a first polynucleotide encoding a first zinc finger nuclease, and a second polynucleotide encoding a second zinc finger nuclease disclosed herein, for use in integrating a transgene into a target nucleotide sequence of a cell.

In another aspect, provided herein is any of the push-pull donor polynucleotide constructs and a polynucleotide encoding one or more zinc finger nucleases disclosed herein, for use in integrating a transgene into a target nucleotide sequence of a cell.

In another aspect, provided herein is any of the push-pull donor polynucleotide constructs and a polynucleotide encoding a 2-in-1 zinc finger nuclease disclosed herein, for use in for integrating a transgene into a target nucleotide sequence of a cell.

In another aspect, provided herein is any of the push-pull donor polynucleotide constructs disclosed herein, for use in disrupting a target nucleotide sequence in a cell.

In another aspect, provided herein is any of the push-pull donor polynucleotide constructs, a first polynucleotide encoding a first zinc finger nuclease, and a second polynucleotide encoding a second zinc finger nuclease disclosed herein, for use in disrupting a target nucleotide sequence in a cell.

In another aspect, provided herein is any of the push-pull donor polynucleotide constructs and a polynucleotide encoding one or more zinc finger nucleases disclosed herein, for use in disrupting a target nucleotide sequence in a cell.

In another aspect, provided herein is any of the push-pull donor polynucleotide constructs and a polynucleotide encoding a 2-in-1 zinc finger nuclease disclosed herein, for use in disrupting a target nucleotide sequence in a cell.

In another aspect, provided herein is any of the push-pull donor polynucleotide constructs disclosed herein, for use in correcting a disease-causing mutation in the genome of a cell.

In another aspect, provided herein is any of the push-pull donor polynucleotide constructs, a first polynucleotide encoding a first zinc finger nuclease, and a second polynucleotide encoding a second zinc finger nuclease disclosed herein, for use in correcting a disease-causing mutation in the genome of a cell.

In another aspect, provided herein is any of the push-pull donor polynucleotide constructs and a polynucleotide encoding one or more zinc finger nucleases disclosed herein, for use in correcting a disease-causing mutation in the genome of a cell.

In another aspect, provided herein is any of the push-pull donor polynucleotide constructs and a polynucleotide encoding a 2-in-1 zinc finger nuclease disclosed herein, for use in correcting a disease-causing mutation in the genome of a cell.

In another aspect, provided herein is any of the push-pull donor polynucleotide constructs disclosed herein, for use in modifying a target nucleotide sequence in the genome of a cell.

In another aspect, provided herein is any of the push-pull donor polynucleotide constructs, a first polynucleotide encoding a first zinc finger nuclease, and a second polynucleotide encoding a second zinc finger nuclease disclosed herein, for use in modifying a target nucleotide sequence in the genome of a cell.

In another aspect, provided herein is any of the push-pull donor polynucleotide constructs and a polynucleotide encoding one or more zinc finger nucleases disclosed herein, for use in modifying a target nucleotide sequence in the genome of a cell.

In another aspect, provided herein is any of the push-pull donor polynucleotide constructs and a polynucleotide encoding a 2-in-1 zinc finger nuclease disclosed herein, for use in modifying a target nucleotide sequence in the genome of a cell.

In another aspect, provided herein is any of the vectors disclosed herein for use in treating a disease or disorder.

In another aspect, provided herein is any of the vectors comprising a push-pull donor polynucleotide construct disclosed herein, for use in treating a disease or disorder.

In another aspect, provided herein is any of the vectors comprising a push-pull donor polynucleotide construct, a vector comprising a first polynucleotide encoding a first zinc finger nuclease, and a vector comprising a second polynucleotide encoding a second zinc finger nuclease disclosed herein, for use in treating a disease or disorder.

In another aspect, provided herein is any of the vectors comprising a push-pull donor polynucleotide construct and a vector comprising a polynucleotide encoding one or more zinc finger nucleases disclosed herein, for use in treating a disease or disorder.

In another aspect, provided herein is any of the vectors comprising a push-pull donor polynucleotide construct and a vector comprising a polynucleotide encoding a 2-in-1 zinc finger nuclease disclosed herein, for use in for treating a disease or disorder.

In another aspect, provided herein is any of the vectors disclosed herein, for use in modifying the genome of a cell.

In another aspect, provided herein is any of the vectors comprising a push-pull donor polynucleotide construct disclosed herein, for use in modifying the genome of a cell.

In another aspect, provided herein is any of the vectors comprising a push-pull donor polynucleotide construct, a vector comprising a first polynucleotide encoding a first zinc finger nuclease, and a vector comprising a second polynucleotide encoding a second zinc finger nuclease disclosed herein, for use in modifying the genome of a cell.

In another aspect, provided herein is any of the vectors comprising a push-pull donor polynucleotide construct and a vector comprising a polynucleotide encoding one or more zinc finger nucleases disclosed herein, for use in modifying the genome of a cell.

In another aspect, provided herein is any of the vectors comprising a push-pull donor polynucleotide construct and a vector comprising a polynucleotide encoding a 2-in-1 zinc finger nuclease disclosed herein, for use in modifying the genome of a cell.

In another aspect, provided herein is any of the vectors disclosed herein, for use in integrating a transgene into a target nucleotide sequence of a cell.

In another aspect, provided herein is any of the vectors comprising a push-pull donor polynucleotide construct disclosed herein, for use in integrating a transgene into a target nucleotide sequence of a cell.

In another aspect, provided herein is any of the vectors comprising a push-pull donor polynucleotide construct, a vector comprising a first polynucleotide encoding a first zinc finger nuclease, and a vector comprising a second polynucleotide encoding a second zinc finger nuclease disclosed herein, for use in integrating a transgene into a target nucleotide sequence of a cell.

In another aspect, provided herein is any of the vectors comprising a push-pull donor polynucleotide construct and a vector comprising a polynucleotide encoding one or more zinc finger nucleases disclosed herein, for use in integrating a transgene into a target nucleotide sequence of a cell.

In another aspect, provided herein is any of the vectors comprising a push-pull donor polynucleotide constructs and a vector comprising a polynucleotide encoding a 2-in-1 zinc finger nuclease disclosed herein, for use in integrating a transgene into a target nucleotide sequence of a cell.

In another aspect, provided herein is any of the vectors disclosed herein, for use in disrupting a target nucleotide sequence in a cell.

In another aspect, provided herein is any of the vectors comprising a push-pull donor polynucleotide construct disclosed herein, for use in disrupting a target nucleotide sequence in a cell.

In another aspect, provided herein is any of the vectors comprising a push-pull donor polynucleotide construct, a vector comprising a first polynucleotide encoding a first zinc finger nuclease, and a vector comprising a second polynucleotide encoding a second zinc finger nuclease disclosed herein, for use in disrupting a target nucleotide sequence in a cell.

In another aspect, provided herein is any of the vectors comprising a push-pull donor polynucleotide construct and a vector comprising a polynucleotide encoding one or more zinc finger nucleases disclosed herein, for use in disrupting a target nucleotide sequence in a cell.

In another aspect, provided herein is any of the vectors comprising a push-pull donor polynucleotide construct and a vector comprising polynucleotide encoding a 2-in-1 zinc finger nuclease disclosed herein, for use in disrupting a target nucleotide sequence in a cell.

In another aspect, provided herein is any of the vectors disclosed herein, for use in for correcting a disease-causing mutation in the genome of a cell.

In another aspect, provided herein is any of the vectors comprising a push-pull donor polynucleotide construct disclosed herein, use in correcting a disease-causing mutation in the genome of a cell.

In another aspect, provided herein is any of the vectors comprising a push-pull donor polynucleotide constructs, a vector comprising a first polynucleotide encoding a first zinc finger nuclease, and a vector comprising a second polynucleotide encoding a second zinc finger nuclease disclosed herein, for use in correcting a disease-causing mutation in the genome of a cell.

In another aspect, provided herein is any of the vectors comprising a push-pull donor polynucleotide construct and a vector comprising a polynucleotide encoding one or more zinc finger nucleases disclosed herein, for use in correcting a disease-causing mutation in the genome of a cell.

In another aspect, provided herein is any of the vectors comprising a push-pull donor polynucleotide construct and a vector comprising a polynucleotide encoding a 2-in-1 zinc finger nuclease disclosed herein, for use in correcting a disease-causing mutation in the genome of a cell.

In another aspect, provided herein is any of the vectors disclosed herein, for use in modifying a target nucleotide sequence in the genome of a cell.

In another aspect, provided herein is any of the vectors comprising the push-pull donor polynucleotide constructs disclosed herein, for use in modifying a target nucleotide sequence in the genome of a cell.

In another aspect, provided herein is any of the vectors comprising a push-pull donor polynucleotide construct, a vector comprising a first polynucleotide encoding a first zinc finger nuclease, and a vector comprising a second polynucleotide encoding a second zinc finger nuclease disclosed herein, for use in modifying a target nucleotide sequence in the genome of a cell.

In another aspect, provided herein is any of the vector comprising a push-pull donor polynucleotide construct and a vector comprising a polynucleotide encoding one or more zinc finger nucleases disclosed herein, for use in modifying a target nucleotide sequence in the genome of a cell.

In another aspect, provided herein is any of the vectors comprising a push-pull donor polynucleotide construct and a vector comprising a polynucleotide encoding a 2-in-1 zinc finger nuclease disclosed herein, use in modifying a target nucleotide sequence in the genome of a cell.

### Exemplary Constructs

Non-limiting examples of push-pull donor constructs include constructs as shown in Table 2; and constructs comprising one or more of the sequences of Table 3 in any order or combination.

TABLE 2
Exemplary Push-Pull Donor

| Const | SEQ ID NO | Sequence |
|---|---|---|
| IDS_ | 173 | [CTGCGCGCTCGCTCGCTCACTGAGGCCGCCCGGGCAAAGCCCGGGCGTCGGGC |
| push_ | | GACCTTTGGTCGCCCGGCCTCAGTGAGCGAGCGAGCGCGCAGAGAGGGAGTGGC |
| pull_1 | | 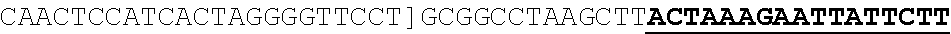 |
| | | 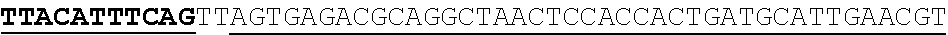 |
| | | CCTCCTTATCATTGTTGACGATCTTCGACCCTCTTTGGGCTGCTACGGCGACAA |
| | | ACTGGTTCGCAGCCCCAACATAGACCAGCTTGCTTCCCATTCACTGCTTTTTCA |
| | | GAACGCGTTTGCTCAGCAAGCCGTCTGCGCACCATCCCGCGTTTCTTTTCTTAC |
| | | TGGACGACGCCCTGACACGACCCGACTGTACGATTTTAATAGTTACTGGCGCGT |
| | | TCATGCCGGCAATTTCTCAACCATCCCTCAGTACTTCAAAGAGAACGGATACGT |
| | | CACCATGAGCGTTGGCAAGGTGTTCCATCCAGGCATCTCTTCCAACCATACCGA |
| | | CGATAGCCCATACAGCTGGTCCTTTCCCCCATATCATCCCTCAAGTGAAAAATA |
| | | TGAAAATACAAAGACATGCAGAGGTCCCGACGGCGAGCTTCACGCCAATCTCCT |
| | | GTGTCCAGTTGATGTGCTCGATGTGCCAGAGGGGACACTCCCTGATAAACAATC |
| | | TACTGAGCAGGCTATCCAGCTCCTTGAGAAAATGAAAACCTCTGCCAGCCCCTT |
| | | TTTCTTGGCCGTCGGTTACCACAAGCCCCACATTCCATTCCGGTATCCAAAAGA |
| | | ATTCCAGAAATTGTATCCTCTTGAAAACATCACCCTGGCCCCCGACCCTGAAGT |
| | | GCCCGATGGCCTGCCCCCTGTCGCCTATAACCCATGGATGGATATCAGGCAGAG |
| | | AGAGGACGTGCAGGCCCTTAATATCTCAGTTCCCTACGGACCAATTCCCGTTGA |
| | | TTTTCAAAGAAAGATCCGCCAGTCCTACTTTGCTAGCGTCTCATACCTCGACAC |
| | | ACAGGTCGGCAGACTTCTCAGCGCCCTCGACGACCTGCAATTGGCTAACAGCAC |
| | | CATCATTGCCTTCACCTCTGACCACGGGTGGGCGCTCGGCGAACACGGCGAGTG |
| | | GGCCAAATATTCAAATTTCGACGTCGCCACACACGTACCCCTTATCTTTTACGT |
| | | CCCCGGTAGAACCGCTAGTCTGCCCGAAGCAGGAGAGAAACTGTTCCCCTATCT |
| | | GGACCCCTTTGATTCAGCTAGCCAATTGATGGAGCCCGGTAGACAATCCATGGA |
| | | TTTGGTTGAACTCGTGTCCCTCTTTCCCACGCTGGCCGGTCTGGCCGGTCTCCA |
| | | AGTTCCCCCCAGGTGCCCCGTTCCTTCTTTCCACGTAGAGCTGTGCAGGGAGGG |
| | | AAAAAACTTGCTTAAACATTTTCGGTTTCGCGACCTGGAGGAAGACCCCTACTT |
| | | GCCCGGTAATCCCCGCGAGCTGATCGCTTATTCCCAATACCCTAGACCTAGCGA |
| | | CATCCCTCAGTGGAATTCCGATAAGCCGTCCCTCAAGGACATTAAGATTATGGG |
| | | ATACTCTATTCGCACTATTGACTACAGATATACCGTCTGGGTGGGCTTCAATCC |
| | | TGATGAATTCCTGGCAAACTTTTCCGATATTCACGCTGGTGAGCTGTATTTCGT |
| | | CGAGTCCGATCCACTGCAAGACCACAATATGTACAACGATTCCCAAGGCGGAGA |
| | | TTTGTTCCAGCTCTTGATGCCTTGATAAAGATCTCTGTGCCTTCTAGTTGCCAG |
| | | CCATCTGTTGTTTGCCCCTCCCCCGTGCCTTCCTTGACCCTGGAAGGTGCCACT |
| | | CCCACTGTCCTTTCCTAATAAAATGAGGAAATTGCATCGCATTGTCTGAGTAGG |
| | | TGTCATTCTATTCTGGGGGGTGGGGTGGGGCAGGACAGCAAGGGGGAGGATTGG |
| | | 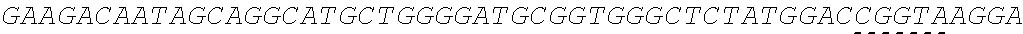 |
| | | 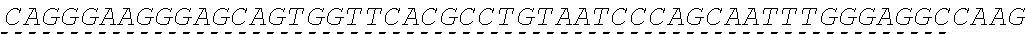 |
| | | 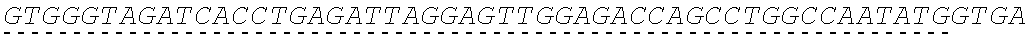 |
| | | 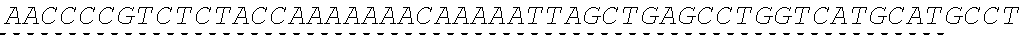 |
| | | 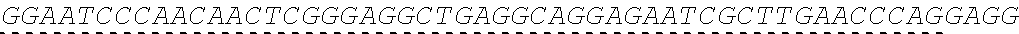 |
| | | 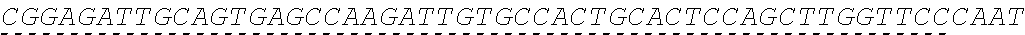 |
| | | 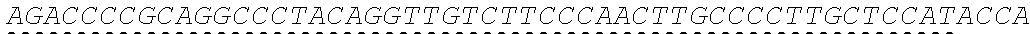 |
| | | 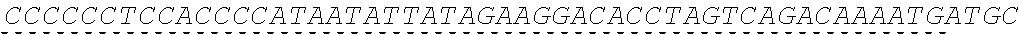 |
| | | 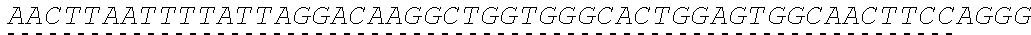 |
| | | 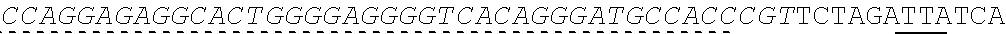 |
| | | CGGCATGAGCAGCTGGAACAAATCTCCTCCTTGGGAATCATTATACATATTGTG |
| | | ATCTTGCAACGGGTCCGAGTCTACGAAATACAGCTCACCAGCGTGGATGTCCGA |
| | | AAAGTTCGCGAGGAATTCGTCAGGATTGAACCCTACCCACACTGTGTAGCGATA |
| | | GTCGATGGTCCTGATCGAGTACCCCATAATCTTGATGTCTTTGAGGGAGGGCTT |
| | | ATCGGAGTTCCATTGAGGAATATCGCTGGGTCGCGGATACTGGGAATAGGCAAT |
| | | CAACTCTCGCGGATTCCCTGGCAGATAGGGGTCCTCCTCAAGGTCCCTGAACCG |
| | | AAAGTGTTTGAGGAGGTTTTTCCCTTCGCGGCAGAGTTCCACATGGAAGCTCGG |
| | | TACAGGGCATCTAGGGGGTACTTGCAAGCCCGCCAACCCGGCGAGGGTCGGAAA |
| | | AAGGGACACCAATTCTACCAAGTCCATGGATTGTCTGCCCGGTTCCATAAGCTG |
| | | GCTCGCCGAGTCGAATGGATCGAGATAGGGAAAAAGTTTTTCGCCTGCCTCGGG |
| | | AAGCGAGGCCGTTCTACCCGGCACGTAGAAAATCAGGGGCACGTGCGTTGCTAC |
| | | ATCAAAATTGCTATACTTTGCCCACTCTCCATGCTCTCCCAACGCCCACCCATG |
| | | GTCCGACGTAAAGGCGATGATTGTGGAATTTGCCAGCTGAAGGTCATCAAGCGC |
| | | GCTCAGAAGTCGACCTACTTGCGTATCGAGGTAGGACACCGACGCAAAATACGA |
| | | CTGCCGAATCTTGCGTTGAAAATCGACTGGAATAGGCCCGTAGGGGACTGAGAT |
| | | GTTGAGTGCCTGCACATCTTCCCTCTGCCTGATATCCATCCAGGGATTGTAGGC |
| | | CACGGGTGGCAGACCGTCGGGGACTTCCGGGTCCGGTGCCAAAGTGATGTTTTC |
| | | CAAAGGATAAAGTTTCTGGAACTCCTTCGGGTAGCGGAAAGGAATATGGGGCTT |
| | | GTGATACCCCACGGCGAGGAAGAAAGGCGACGCGCTTGTTTTCATCTTCTCCAG |
| | | CAACTGAATCGCCTGCTCCGTTGACTGCTTGTCGGGGAGCGTTCCCTCGGGCAC |
| | | GTCCAAGACATCCACCGGACACAGCAGATTAGCGTGCAGCTCTCCGTCGGGTCC |
| | | GCGACAAGTTTTCGTGTTCTCATACTTCTCGCTCGAAGGATGGTAGGGAGGAAA |
| | | CGACCACGAGTAGGGCGAATCGTCGGTGTGATTCGAGGAGATGCCGGGGTGAAA |
| | | GACCTTTCCCACGCTCATTGTCACGTATCCGTTCTCTTTAAAGTACTGTGGGAT |
| | | AGTTGAAAAGTTACCCGCGTGGACTCTCCAGTAGCTGTTGAAGTCGTACAGCCG |
| | | CGTTGTGTCAGGGCGTCGCCCGGTCAAGAATGAGACTCTTGAAGGTGCACAGAC |
| | | AGCCTGCTGCGCAAACGCATTTTGGAAAAGCAGTGAGTGTGAGGCCAACTGATC |
| | | GATGTTCGGCGAGCGGACGAGCTTATCTCCATAGCAGCCAAGCGACGGCCGCAA |
| | | ATCGTCCACGATGATGAGCAGGACGTTAAGCGCATCTGTAGTTGAGTTGGCCTG |
| | | 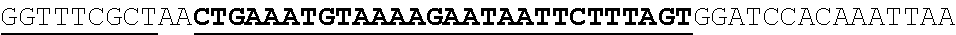 |
| | | TCGAACCTGCAGCTGATATCGACGCTTAAGTAGGGCTTAGCAAACGCGTCTCCA |
| | | ACGTTTCGCCGTTAACACCCCACATAGTGAGTGGTCTTAGTAGTCCGGGTGTTT |
| | | AAACTGAAAGATAACTCGAGCGC[AGGAACCCCTAGTGATGGAGTTGGCCACTC |
| | | CCTCTCTGCGCGCTOGOTOGOTCACTGAGGCCGCCCGGGCTTTGCCCGGGCGGC |
| | | CTCAGTGAGCGAGCGAGCGCGCAG] |
| IDS_ | 174 | [CTGCGCGCTCGCTCGCTCACTGAGGCCGCCCGGGCAAAGCCCGGGCGTCGGGC |
| push_ | | GACCTTTGGTCGCCCGGCCTCAGTGAGCGAGCGAGCGCGCAGAGAGGGAGTGGC |
| pull_2 | | 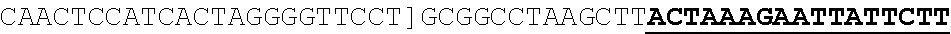 |
| | | 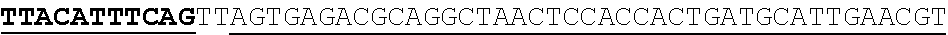 |
| | | TTTGCTGATTATAGTGGATGACCTCAGACCTTCACTCGGCTGTTACGGTGACAA |
| | | ACTGGTCCGCTCTCCGAATATCGACCAACTGGCAAGCCACTCCCTCCTTTTCCA |
| | | AAACGCATTCGCTCAACAAGCAGTTTGTGCCCCCAGTAGAGTGTCCTTCTTGAC |
| | | TGGTCGCAGGCCCGACACCACCCGCCTGTACGATTTTAACTCATATTGGCGCGT |
| | | TCATGCCGGCAACTTTTCTACAATACCACAATACTTTAAGGAAAATGGCTACGT |
| | | AACTATGAGTGTGGGCAAGGTGTTTCACCCCGGTATTTCAAGCAATCACACAGA |
| | | CGACTCTCCCTACTCCTGGTCCTTTCCCCCATACCATCCTTCCTCAGAGAAGTA |
| | | CGAAAATACCAAGACGTGTAGAGGTCCGGACGGCGAACTGCACGCAAACCTGTT |
| | | GTGCCCTGTTGACGTACTCGACGTCCCGGAAGGCACCCTCCCCGACAAGCAATC |
| | | TACCGAGCAGGCCATTCAGCTCCTCGAAAAGATGAAAACAAGTGCATCCCCCTT |
| | | TTTCCTGGCTGTAGGTTATCATAAACCCCACATTCCATTCCGGTATCCTAAAGA |
| | | ATTTCAGAAGCTGTACCCCCTTGAAAACATTAGACTGGCACCAGACCCAGAAGT |
| | | CCCAGACGGACTCCCCCCAGTGGCCTATAACCCATGGATGGACATCAGGCAGCG |
| | | CGAAGACGTGCAGGCTCTTAACATCAGCGTCCCATATGGCCCAATACCTGTCGA |
| | | CTTTCAACGCAAGATTAGACAATCCTATTTCGCTTCTGTGAGTTACCTGGACAC |
| | | ACAAGTAGGAAGACTGCTCAGCGCCCTTGACGATCTGCAACTCGCTAATTCTAC |
| | | CATAATTGCCTTTACCAGCGACCATGGATGGGCACTCGGAGAACACGGCGAATG |
| | | GGCAAAGTACTCCAATTTCGATGTCGCAACCCACGTTCCCTTGATATTCTATGT |
| | | CCCCGGCCGCACTGCGTCCTTGCCAGAAGCTGGGGAAAAACTCTTTCCATATCT |
| | | GGACCCCTTCGACTCTGCATCCCAACTGATGGAACCCGGTAGACAAAGTATGGA |
| | | TCTGGTCGAGCTCGTTTCACTCTTTCCGACCCTTGCCGGTCTCGCCGGCCTTCA |
| | | GGTGCCACCACGATGCCCCGTTCCGAGCTTCCACGTCGAGCTTTGTAGAGAAGG |
| | | GAAAAACCTCCTGAAACATTTCCGATTTCGCGACCTGGAGGAAGACCCATACCT |
| | | GCCCGGGAATCCTAGAGAACTCATCGCATATTCTCAGTACCCCAGACCCTCCGA |
| | | CATCCCACAGTGGAACTCTGACAAACCATCTTTGAAAGACATTAAGATTATGGG |
| | | CTACAGCATCCGGACTATAGATTACAGGTATACCGTATGGGTTGGATTCAATCC |
| | | CGATGAATTCCTCGCGAATTTCTCAGACATCCACGCAGGAGAACTCTATTTCGT |
| | | GGACTCAGACCCCCTTCAAGATCACAACATGTACAACGATTCCCAAGGAGGTGA |
| | | TCTTTTTCAGTTGCTCATGCCTTGATAAAGATCTCTGTGCCTTCTAGTTGCCAG |
| | | CCATCTGTTGTTTGCCCCTCCCCCGTGCCTTCCTTGACCCTGGAAGGTGCCACT |
| | | CCCACTGTCCTTTCCTAATAAAATGAGGAAATTGCATCGCATTGTCTGAGTAGG |
| | | TGTCATTCTATTCTGGGGGGTGGGGTGGGGCAGGACAGCAAGGGGGAGGATTGG |
| | | 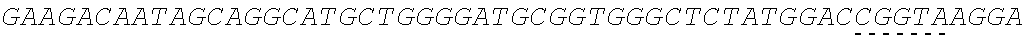 |
| | | 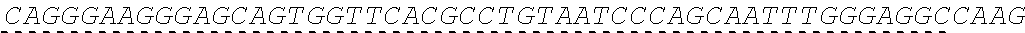 |
| | | 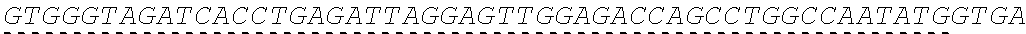 |
| | | 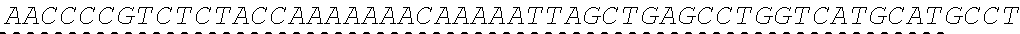 |
| | | 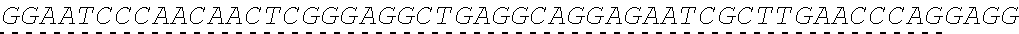 |
| | | 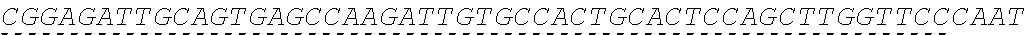 |
| | | 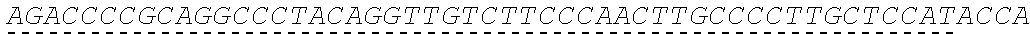 |
| | | 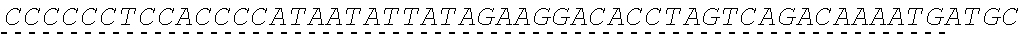 |
| | | 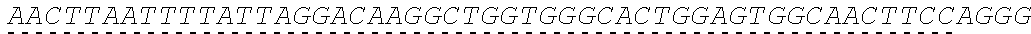 |
| | | 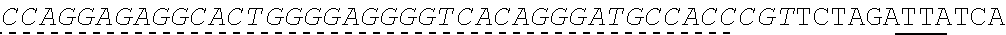 |
| | | CGGCATGAGCAGCTGGAACAAATCTCCTCCTTGGGAATCATTATACATATTGTG |
| | | ATCTTGCAACGGGTCCGAGTCTACGAAATACAGCTCACCAGCGTGGATGTCCGA |
| | | AAAGTTCGCGAGGAATTCGTCAGGATTGAACCCTACCCACACTGTGTAGCGATA |
| | | GTCGATGGTCCTGATCGAGTACCCCATAATCTTGATGTCTTTGAGGGAGGGCTT |
| | | ATCGGAGTTCCATTGAGGAATATCGCTGGGTCGCGGATACTGGGAATAGGCAAT |
| | | CAACTCTCGCGGATTCCCTGGCAGATAGGGGTCCTCCTCAAGGTCCCTGAACCG |
| | | AAAGTGTTTGAGGAGGTTTTTCCCTTCGCGGCAGAGTTCCACATGGAAGCTCGG |
| | | TACAGGGCATCTAGGGGGTACTTGCAAGCCCGCCAACCCGGCGAGGGTCGGAAA |
| | | AAGGGACACCAATTCTACCAAGTCCATGGATTGTCTGCCCGGTTCCATAAGCTG |
| | | GCTCGCCGAGTCGAATGGATCGAGATAGGGAAAAAGTTTTTCGCCTGCCTCGGG |
| | | AAGCGAGGCCGTTCTACCCGGCACGTAGAAAATCAGGGGCACGTGCGTTGCTAC |
| | | ATCAAAATTGCTATACTTTGCCCACTCTCCATGCTCTCCCAACGCCCACCCATG |
| | | GTCCGACGTAAAGGCGATGATTGTGGAATTTGCCAGCTGAAGGTCATCAAGCGC |
| | | GCTCAGAAGTCGACCTACTTGCGTATCGAGGTAGGACACCGACGCAAAATACGA |
| | | CTGCCGAATCTTGCGTTGAAAATCGACTGGAATAGGCCCGTAGGGGACTGAGAT |
| | | GTTGAGTGCCTGCACATCTTCCCTCTGCCTGATATCCATCCAGGGATTGTAGGC |
| | | CACGGGTGGCAGACCGTCGGGGACTTCCGGGTCCGGTGCCAAAGTGATGTTTTC |
| | | CAAAGGATAAAGTTTCTGGAACTCCTTCGGGTAGCGGAAAGGAATATGGGGCTT |
| | | GTGATACCCCACGGCGAGGAAGAAAGGCGACGCGCTTGTTTTCATCTTCTCCAG |
| | | CAACTGAATCGCCTGCTCCGTTGACTGCTTGTCGGGGAGCGTTCCCTCGGGCAC |
| | | GTCCAAGACATCCACCGGACACAGCAGATTAGCGTGCAGCTCTCCGTCGGGTCC |
| | | GCGACAAGTTTTCGTGTTCTCATACTTCTCGCTCGAAGGATGGTAGGGAGGAAA |
| | | CGACCACGAGTAGGGCGAATCGTCGGTGTGATTCGAGGAGATGCCGGGGTGAAA |
| | | GACCTTTCCCACGCTCATTGTCACGTATCCGTTCTCTTTAAAGTACTGTGGGAT |
| | | AGTTGAAAAGTTACCCGCGTGGACTCTCCAGTAGCTGTTGAAGTCGTACAGCCG |
| | | CGTTGTGTCAGGGCGTCGCCCGGTCAAGAATGAGACTCTTGAAGGTGCACAGAC |
| | | AGCCTGCTGCGCAAACGCATTTTGGAAAAGCAGTGAGTGTGAGGCCAACTGATC |
| | | GATGTTCGGCGAGCGGACGAGCTTATCTCCATAGCAGCCAAGCGACGGCCGCAA |
| | | ATCGTCCACGATGATGAGCAGGACGTTAAGCGCATCTGTAGTTGAGTTGGCCTG |
| | | 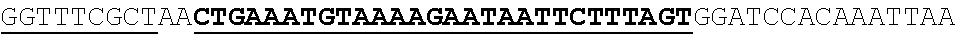 |
| | | TCGAACCTGCAGCTGATATCGACGCTTAAGTAGGGCTTAGCAAACGCGTCTCCA |
| | | ACGTTTCGCCGTTAACACCCCACATAGTGAGTGGTCTTAGTAGTCCGGGTGTTT |
| | | AAACTGAAAGATAACTCGAGCGC[AGGAACCCCTAGTGATGGAGTTGGCCACTC |
| | | CCTCTCTGCGCGCTOGOTOGOTCACTGAGGCCGCCCGGGCTTTGCCCGGGCGGC |
| | | CTCAGTGAGCGAGCGAGCGCGCAG] |
| IDS_ | 175 | [CTGCGCGCTCGCTCGCTCACTGAGGCCGCCCGGGCAAAGCCCGGGCGTCGGGC |
| push_ | | GACCTTTGGTCGCCCGGCCTCAGTGAGCGAGCGAGCGCGCAGAGAGGGAGTGGC |
| pull_4 | | 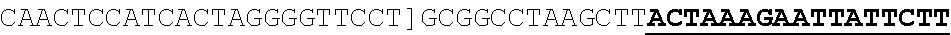 |
| | | 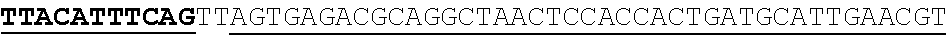 |
| | | TCTGCTTATTATCGTGGATGATCTGCGACCCTCACTTGGTTGCTATGGCGATAA |
| | | ATTGGTTAGAAGTCCGAACATAGACCAGCTGGCGAGTCATTCTCTCCTCTTCCA |
| | | AAACGCGTTCGCACAACAGGCCGTTTGCGCCCCTTCAAGAGTATCCTTTCTGAC |
| | | AGGCAGACGCCCCGATACTACTAGGCTGTATGACTTCAATTCCTACTGGCGCGT |
| | | GCACGGAGGTAATTTCTCTACAATCCCCGAGTACTTCAAAGAAAACGGATACGT |
| | | TACCATGAGCGTCGGCAAAGTGTTCCATCCCGGAATTTCTAGCAACCATACGGA |
| | | TGACAGCCCCTATTCCTGGTCATTTCCACCGTACCATCCTTCCAGTGAAAAATA |
| | | TGAGAACACTAAAACTTGTCGCGGACCTGACGGAGAATTGCACGCAAACCTTCT |
| | | CTGCCCCGTAGATGTGCTCGATGTGCCTGAAGGAACTCTCCCAGACAAGCAGAG |
| | | TACCGAACAAGCCATTCAGCTGCTGGAAAAGATGAAAACGTCCGCCTCACCTTT |
| | | CTTCCTCGCAGTCGGTTACCACAAGCCCCACATTCCTTTTAGATACCCTAAAGA |
| | | GTTTCAGAAACTGTATCCCCTTGAAAATATCACCCTCGCTCCCGACCCCGAGGT |
| | | CCCGGACGGCCTGCCCCCTGTTGCATACAACCCCTGGATGGATATCAGACAACG |
| | | GGAGGATGTTCAAGCACTCAACATCTCAGTACCATACGGCCCAATCCCTGTCGA |
| | | TTTCCAAAGGAAAATCAGGCAGTCCTACTTTGCAAGCGTGTCTTATCTCGACAC |
| | | CCAGGTCGGAAGACTGCTGTCCGCCCTCGACGACCTTCAATTGGCTAACTCTAC |
| | | AATCATTGCCTTCACTAGCGATCACGGGTGGGCGCTTGGCGAGCACGGAGAATG |
| | | GGCCAAATACTCTAATTTTGATGTTGCCACCCACGTGCCCCTCATATTTTATGT |
| | | TCCAGGTAGAACCGCAAGCCTGCCAGAAGCCGGTGAGAAGCTGTTTCCTTACCT |
| | | CGATCCTTTCGATAGTGCATCCCAACTGATGGAGCCAGGTCGACAATCTATGGA |
| | | CCTGGTAGAGCTGGTCTCTCTGTTCCCAACGCTCGCCGGACTTGCTGGACTGCA |
| | | GGTGCCACCCCGCTGCCCTGTACCCTCCTTCCACGTTGAGCTCTGCCGCGAAGG |
| | | CAAGAACCTGTTGAAACATTTTCGATTCAGAGACCTTGAAGAGGAGCCATACCT |
| | | CCCAGGAAATCCAAGAGAGCTGATTGCTTATTCTCAATATCCCAGGCCCAGTGA |
| | | CATACCACAGTGGAATAGCGATAAACCCTCACTTAAAGACATTAAGATAATGGG |
| | | CTATTCCATCCGGACAATTGATTACAGATACACAGTTTGGGTGGGGTTTAACCC |
| | | AGACGAATTCCTTGCGAATTTCAGCGATATTCATGCCGGAGAACTTTATTTTGT |
| | | TGATAGCGACCCCCTCCAGGACCACAACATGTACAACGACTCACAGGGTGGCGA |
| | | TCTCTTTCAGCTCCTGATGCCGTGATAAAGATCTCTGTGCCTTCTAGTTGCCAG |
| | | CCATCTGTTGTTTGCCCCTCCCCCGTGCCTTCCTTGACCCTGGAAGGTGCCACT |
| | | CCCACTGTCCTTTCCTAATAAAATGAGGAAATTGCATCGCATTGTCTGAGTAGG |
| | | TGTCATTCTATTCTGGGGGGTGGGGTGGGGCAGGACAGCAAGGGGGAGGATTGG |
| | | 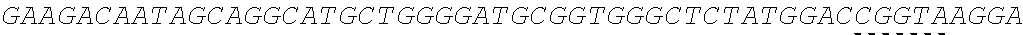 |
| | | 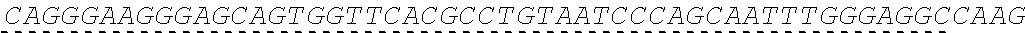 |
| | | 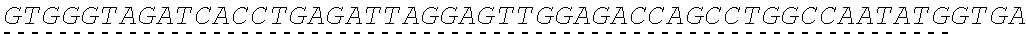 |
| | | 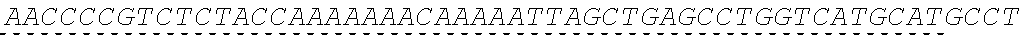 |
| | | 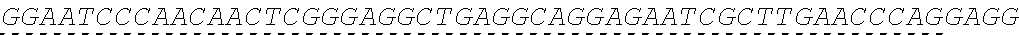 |
| | | 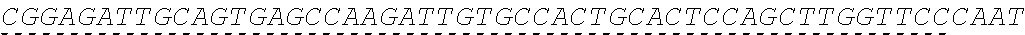 |
| | | 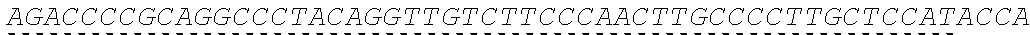 |
| | | 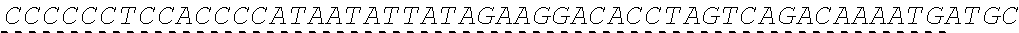 |
| | | 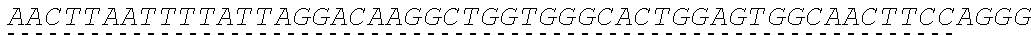 |
| | | 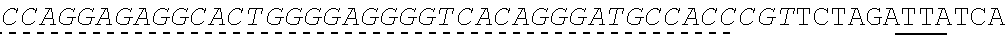 |
| | | CGGCATGAGCAGCTGGAACAAATCTCCTCCTTGGGAATCATTATACATATTGTG |
| | | ATCTTGCAACGGGTCCGAGTCTACGAAATACAGCTCACCAGCGTGGATGTCCGA |
| | | AAAGTTCGCGAGGAATTCGTCAGGATTGAACCCTACCCACACTGTGTAGCGATA |
| | | GTCGATGGTCCTGATCGAGTACCCCATAATCTTGATGTCTTTGAGGGAGGGCTT |
| | | ATCGGAGTTCCATTGAGGAATATCGCTGGGTCGCGGATACTGGGAATAGGCAAT |
| | | CAACTCTCGCGGATTCCCTGGCAGATAGGGGTCCTCCTCAAGGTCCCTGAACCG |
| | | AAAGTGTTTGAGGAGGTTTTTCCCTTCGCGGCAGAGTTCCACATGGAAGCTCGG |
| | | TACAGGGCATCTAGGGGGTACTTGCAAGCCCGCCAACCCGGCGAGGGTCGGAAA |
| | | AAGGGACACCAATTCTACCAAGTCCATGGATTGTCTGCCCGGTTCCATAAGCTG |
| | | GCTCGCCGAGTCGAATGGATCGAGATAGGGAAAAAGTTTTTCGCCTGCCTCGGG |
| | | AAGCGAGGCCGTTCTACCCGGCACGTAGAAAATCAGGGGCACGTGCGTTGCTAC |
| | | ATCAAAATTGCTATACTTTGCCCACTCTCCATGCTCTCCCAACGCCCACCCATG |
| | | GTCCGACGTAAAGGCGATGATTGTGGAATTTGCCAGCTGAAGGTCATCAAGCGC |
| | | GCTCAGAAGTCGACCTACTTGCGTATCGAGGTAGGACACCGACGCAAAATACGA |
| | | CTGCCGAATCTTGCGTTGAAAATCGACTGGAATAGGCCCGTAGGGGACTGAGAT |
| | | GTTGAGTGCCTGCACATCTTCCCTCTGCCTGATATCCATCCAGGGATTGTAGGC |
| | | CACGGGTGGCAGACCGTCGGGGACTTCCGGGTCCGGTGCCAAAGTGATGTTTTC |
| | | CAAAGGATAAAGTTTCTGGAACTCCTTCGGGTAGCGGAAAGGAATATGGGGCTT |
| | | GTGATACCCCACGGCGAGGAAGAAAGGCGACGCGCTTGTTTTCATCTTCTCCAG |
| | | CAACTGAATCGCCTGCTCCGTTGACTGCTTGTCGGGGAGCGTTCCCTCGGGCAC |
| | | GTCCAAGACATCCACCGGACACAGCAGATTAGCGTGCAGCTCTCCGTCGGGTCC |
| | | GCGACAAGTTTTCGTGTTCTCATACTTCTCGCTCGAAGGATGGTAGGGAGGAAA |
| | | CGACCACGAGTAGGGCGAATCGTCGGTGTGATTCGAGGAGATGCCGGGGTGAAA |
| | | GACCTTTCCCACGCTCATTGTCACGTATCCGTTCTCTTTAAAGTACTGTGGGAT |
| | | AGTTGAAAAGTTACCCGCGTGGACTCTCCAGTAGCTGTTGAAGTCGTACAGCCG |
| | | CGTTGTGTCAGGGCGTCGCCCGGTCAAGAATGAGACTCTTGAAGGTGCACAGAC |
| | | AGCCTGCTGCGCAAACGCATTTTGGAAAAGCAGTGAGTGTGAGGCCAACTGATC |
| | | GATGTTCGGCGAGCGGACGAGCTTATCTCCATAGCAGCCAAGCGACGGCCGCAA |
| | | ATCGTCCACGATGATGAGCAGGACGTTAAGCGCATCTGTAGTTGAGTTGGCCTG |
| | | 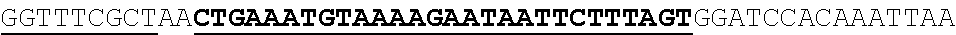 |
| | | TCGAACCTGCAGCTGATATCGACGCTTAAGTAGGGCTTAGCAAACGCGTCTCCA |
| | | ACGTTTCGCCGTTAACACCCCACATAGTGAGTGGTCTTAGTAGTCCGGGTGTTT |
| | | AAACTGAAAGATAACTCGAGCGC[AGGAACCCCTAGTGATGGAGTTGGCCACTC |
| | | CCTCTCTGCGCGCTOGOTOGOTCACTGAGGCCGCCCGGGCTTTGCCCGGGCGGC |
| | | CTCAGTGAGCGAGCGAGCGCGCAG] |
| IDS_ | 176 | [CTGCGCGCTCGCTCGCTCACTGAGGCCGCCCGGGCAAAGCCCGGGCGTCGGGC |
| push_ | | GACCTTTGGTCGCCCGGCCTCAGTGAGCGAGCGAGCGCGCAGAGAGGGAGTGGC |
| pull_5 | | 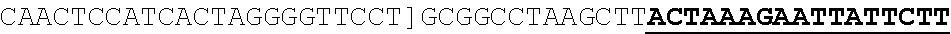 |
| | | 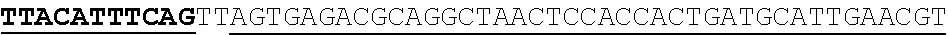 |
| | | TCTGCTTATCATTGTAGATGACCTTCGCCCCAGTTTGGGATGTTATGGCGATAA |
| | | GCTGGTGCGCTCACCTAATATTGATCAGTTGGCAAGCCATAGCCTTTTGTTCCA |
| | | AAATGCTTTTGCTCAGCAGGCTGTATGTGCACCGAGTAGAGTTTCCTTCCTCAC |
| | | CGGACGCAGACCAGACACCACAAGACTCTACGACTTTAACTCATACTGGAGGGT |
| | | CCACGCTGGGAATTTCAGTACGATCCCGCAGTATTTCAAAGAAAATGGCTACGT |
| | | TACCATGTCCGTCGGCAAGGTGTTTCACCCCGGCATCTCATCAAATCATACAGA |
| | | CGATAGCCCTTATTCTTGGTCTTTCCCTCCTTATCATCCATCCAGCGAAAAATA |
| | | CGAGAACACTAAAACATGTAGAGGTCCAGATGGAGAGCTGCACGCCAACCTGCT |
| | | GTGCCCTGTGGATGTTCTCGACGTACCTGAAGGCACCCTTCCAGACAAACAGAG |
| | | CACCGAACAGGCCATCCAGCTTCTGGAGAAGATGAAGACCAGCGCCTCACCTTT |
| | | CTTCCTCGCCGTAGGCTACCACAAACCGCACATCCCCTTTAGATACCCAAAGGA |
| | | ATTTCAGAAGCTGTACCCCCTGGAAAATATAACATTGGCTCCAGACCCGGAAGT |
| | | GCCCGATGGGTTGCCCCCCGTAGCCTATAATCCTTGGATGGATATTAGACAACG |
| | | GGAAGACGTCCAGGCCCTCAATATTTCTGTCCCTTACGGACCAATCCCTGTTGA |
| | | TTTTCAGAGAAAGATAAGACAGTCCTATTTTGCAAGTGTATCCTACCTTGAGAC |
| | | CCAGGTCGGCCGGCTGTTGTCTGCTCTGGACGACCTGCAACTCGCTAACAGTAC |
| | | AATCATAGCCTTTACTAGCGACCACGGATGGGCTCTGGGAGAACATGGAGAATG |
| | | GGCCAAGTATTCTAACTTCGATGTCGCCACACACGTCCCACTCATATTTTACGT |
| | | TCCTGGTCGAACCGCTAGCCTGCCTGAAGCCGGAGAAAAGCTGTTTCCTTATCT |
| | | CGACCCTTTCGATTCCGCAAGCCAGTTGATGGAACCCGGCCGGCAATCAATGGA |
| | | TCTCGTGGAACTGGTGTCACTTTTTCCTACACTCGCTGGACTCGCTGGCCTTCA |
| | | AGTCCCTCCCCGATGTCCTGTCCCATCATTTCACGTAGAGCTGTGTAGAGAAGG |
| | | GAAGAATCTGCTGAAACACTTCCGGTTCCGGGATCTTGAAGAAGATCCATATCT |
| | | CCCAGGCAACCCTCGCGAACTCATCGCTTATAGCCAGTATCCTCGGCCCAGTGA |
| | | CATACCCCAGTGGAATTCCGACAAACCATCACTTAAAGATATCAAAATTATGGG |
| | | ATACTCCATTCGAACCATAGACTATAGGTACACCGTGTGGGTTGGCTTTAATCC |
| | | AGATGAGTTTTTGGCAAACTTTTCAGACATTCACGCCGGAGAGCTGTATTTTGT |
| | | GGACAGTGACCCTCTGCAGGACCATAATATGTACAACGATTCACAAGGCGGCGA |
| | | CCTCTTCCAACTGCTGATGCCCTGATAAAGATCTCTGTGCCTTCTAGTTGCCAG |
| | | CCATCTGTTGTTTGCCCCTCCCCCGTGCCTTCCTTGACCCTGGAAGGTGCCACT |
| | | CCCACTGTCCTTTCCTAATAAAATGAGGAAATTGCATCGCATTGTCTGAGTAGG |
| | | TGTCATTCTATTCTGGGGGGTGGGGTGGGGCAGGACAGCAAGGGGGAGGATTGG |
| | | 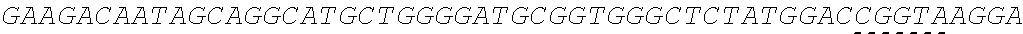 |
| | | 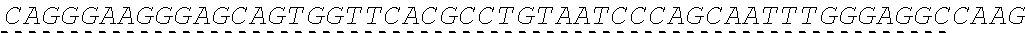 |
| | | 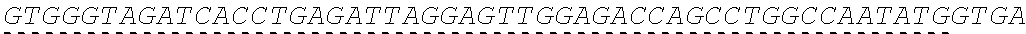 |
| | | 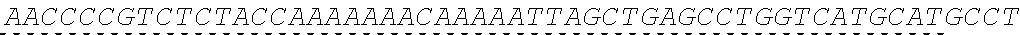 |
| | | 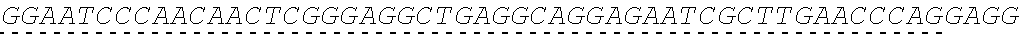 |
| | | 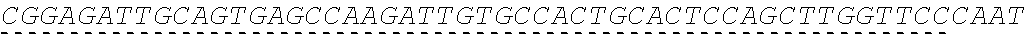 |
| | | 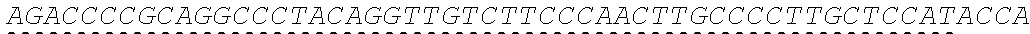 |
| | | 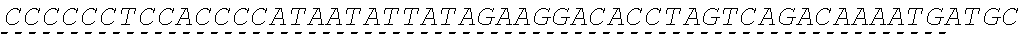 |
| | | 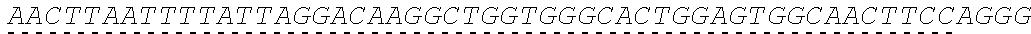 |
| | | 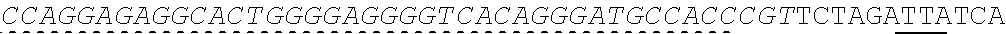 |
| | | CGGCATGAGCAGCTGGAACAAATCTCCTCCTTGGGAATCATTATACATATTGTG |
| | | ATCTTGCAACGGGTCCGAGTCTACGAAATACAGCTCACCAGCGTGGATGTCCGA |
| | | AAAGTTCGCGAGGAATTCGTCAGGATTGAACCCTACCCACACTGTGTAGCGATA |
| | | GTCGATGGTCCTGATCGAGTACCCCATAATCTTGATGTCTTTGAGGGAGGGCTT |
| | | ATCGGAGTTCCATTGAGGAATATCGCTGGGTCGCGGATACTGGGAATAGGCAAT |
| | | CAACTCTCGCGGATTCCCTGGCAGATAGGGGTCCTCCTCAAGGTCCCTGAACCG |
| | | AAAGTGTTTGAGGAGGTTTTTCCCTTCGCGGCAGAGTTCCACATGGAAGCTCGG |
| | | TACAGGGCATCTAGGGGGTACTTGCAAGCCCGCCAACCCGGCGAGGGTCGGAAA |
| | | AAGGGACACCAATTCTACCAAGTCCATGGATTGTCTGCCCGGTTCCATAAGCTG |
| | | GCTCGCCGAGTCGAATGGATCGAGATAGGGAAAAAGTTTTTCGCCTGCCTCGGG |
| | | AAGCGAGGCCGTTCTACCCGGCACGTAGAAAATCAGGGGCACGTGCGTTGCTAC |
| | | ATCAAAATTGCTATACTTTGCCCACTCTCCATGCTCTCCCAACGCCCACCCATG |
| | | GTCCGACGTAAAGGCGATGATTGTGGAATTTGCCAGCTGAAGGTCATCAAGCGC |
| | | GCTCAGAAGTCGACCTACTTGCGTATCGAGGTAGGACACCGACGCAAAATACGA |
| | | CTGCCGAATCTTGCGTTGAAAATCGACTGGAATAGGCCCGTAGGGGACTGAGAT |
| | | GTTGAGTGCCTGCACATCTTCCCTCTGCCTGATATCCATCCAGGGATTGTAGGC |
| | | CACGGGTGGCAGACCGTCGGGGACTTCCGGGTCCGGTGCCAAAGTGATGTTTTC |
| | | CAAAGGATAAAGTTTCTGGAACTCCTTCGGGTAGCGGAAAGGAATATGGGGCTT |
| | | GTGATACCCCACGGCGAGGAAGAAAGGCGACGCGCTTGTTTTCATCTTCTCCAG |
| | | CAACTGAATCGCCTGCTCCGTTGACTGCTTGTCGGGGAGCGTTCCCTCGGGCAC |
| | | GTCCAAGACATCCACCGGACACAGCAGATTAGCGTGCAGCTCTCCGTCGGGTCC |
| | | GCGACAAGTTTTCGTGTTCTCATACTTCTCGCTCGAAGGATGGTAGGGAGGAAA |
| | | CGACCACGAGTAGGGCGAATCGTCGGTGTGATTCGAGGAGATGCCGGGGTGAAA |
| | | GACCTTTCCCACGCTCATTGTCACGTATCCGTTCTCTTTAAAGTACTGTGGGAT |
| | | AGTTGAAAAGTTACCCGCGTGGACTCTCCAGTAGCTGTTGAAGTCGTACAGCCG |
| | | CGTTGTGTCAGGGCGTCGCCCGGTCAAGAATGAGACTCTTGAAGGTGCACAGAC |
| | | AGCCTGCTGCGCAAACGCATTTTGGAAAAGCAGTGAGTGTGAGGCCAACTGATC |
| | | GATGTTCGGCGAGCGGACGAGCTTATCTCCATAGCAGCCAAGCGACGGCCGCAA |
| | | ATCGTCCACGATGATGAGCAGGACGTTAAGCGCATCTGTAGTTGAGTTGGCCTG |
| | | 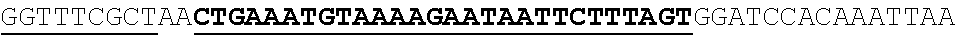 |
| | | TCGAACCTGCAGCTGATATCGACGCTTAAGTAGGGCTTAGCAAACGCGTCTCCA |
| | | ACGTTTCGCCGTTAACACCCCACATAGTGAGTGGTCTTAGTAGTCCGGGTGTTT |
| | | AAACTGAAAGATAACTCGAGCGC[AGGAACCCCTAGTGATGGAGTTGGCCACTC |
| | | CCTCTCTGCGCGCTCGCTCGCTCACTGAGGCCGCCCGGGCTTTGCCCGGGCGGC |
| | | CTCAGTGAGCGAGCGAGCGCGCAG] |

Legend:
5′ ITR = [Text in bracket]
F9SA splice acceptor sequence = Underlined and Bold
bGH polyA = Italicized
hGH polyA = Italicized and Dotted Underline
IDS Transgene = Underlined
3′ ITR = [Bold text in bracket]

TABLE 3
Elements of Exemplary Push-Pull Donor

| SEQ ID NO | Feature/Description | Sequence |
|---|---|---|
| 177 | 5′ ITR | CTGCGCGCTCGCTCGCTCACTGAGGCCGCCCGGGCAAAGCCCG |
| | | GGCGTCGGGCGACCTTTGGTCGCCCGGCCTCAGTGAGCGAGCG |
| | | AGCGCGCAGAGAGGGAGTGGCCAACTCCATCACTAGGGGTTCC |
| | | T |
| 178 | F9SA splice | ACTAAAGAATTATTCTTTTACATTTGAG |
| | acceptor sequence | |
| 179 | bGH | CTGTGCCTTCTAGTTGCCAGCCATCTGTTGTTTGCCCCTCCCC |
| | Polyadenylation | CGTGCCTTCCTTGACCCTGGAAGGTGCCACTCCCACTGTCCTT |
| | signal | TCCTAATAAAATGAGGAAATTGCATCGCATTGTCTGAGTAGGT |
| | | GTCATTCTATTCTGGGGGGTGGGGTGGGGCAGGACAGCAAGGG |
| | | GGAGGATTGGGAAGACAATAGCAGGCATGCTGGGGATGCGGTG |
| | | GGCTCTATGG |
| 180 | hGH | CCTGTGACCCCTCCCCAGTGCCTCTCCTGGCCCTGGAAGTTGC |
| | Polyadenylation | CACTCCAGTGCCCACCAGCCTTGTCCTAATAAAATTAAGTTGC |
| | signal | ATCATTTTGTCTGACTAGGTGTCCTTCTATAATATTATGGGGT |
| | | GGAGGGGGGTGGTATGGAGCAAGGGGCAAGTTGGGAAGACAAC |
| | | CTGTAGGGCCTGCGGGGTCTATTGGGAACCAAGCTGGAGTGCA |
| | | GTGGCACAATCTTGGCTCACTGCAATCTCCGCCTCCTGGGTTC |
| | | AAGCGATTCTCCTGCCTCAGCCTCCCGAGTTGTTGGGATTCCA |
| | | GGCATGCATGACCAGGCTCAGCTAATTTTTGTTTTTTTGGTAG |
| | | AGACGGGGTTTCACCATATTGGCCAGGCTGGTCTCCAACTCCT |
| | | AATCTCAGGTGATCTACCCACCTTGGCCTCCCAAATTGCTGGG |
| | | ATTACAGGCGTGAACCACTGCTCCCTTCCCTGTCCTTGATGCC |
| | | ACCCGT |
| 181 | 3′ ITR | AGGAACCCCTAGTGATGGAGTTGGCCACTCCCTCTCTGCGCGC |
| | | TCGCTCGCTCACTGAGGCCGCCCGGGCTTTGCCCGGGCGGCCT |
| | | CAGTGAGCGAGCGAGCGCGCAG |
| 182 | F9SA splice | CTGAAATGTAAAAGAATAATTCTTTAGT |
| | acceptor sequence | |
| | (reverse | |
| | complement) | |
| 183 | hGH | ACGGGTGGCATCAAGGACAGGGAAGGGAGCAGTGGTTCACGCC |
| | Polyadenylation | TGTAATCCCAGCAATTTGGGAGGCCAAGGTGGGTAGATCACCT |
| | signal | GAGATTAGGAGTTGGAGACCAGCCTGGCCAATATGGTGAAACC |
| | (reverse | CCGTCTCTACCAAAAAAACAAAAATTAGCTGAGCCTGGTCATG |
| | complement) | CATGCCTGGAATCCCAACAACTCGGGAGGCTGAGGCAGGAGAA |
| | | TCGCTTGAACCCAGGAGGCGGAGATTGCAGTGAGCCAAGATTG |
| | | TGCCACTGCACTCCAGCTTGGTTCCCAATAGACCCCGCAGGCC |
| | | CTACAGGTTGTCTTCCCAACTTGCCCCTTGCTCCATACCACCC |
| | | CCCTCCACCCCATAATATTATAGAAGGACACCTAGTCAGACAA |
| | | AATGATGCAACTTAATTTTATTAGGACAAGGCTGGTGGGCACT |
| | | GGAGTGGCAACTTCCAGGGCCAGGAGAGGCACTGGGGAGGGGT |
| | | CACAGG |
| 184 | IDS Transgene | AGTGAGACGCAGGCTAACTCCACCACTGATGCATTGAACGTCC |
| | | TCCTTATCATTGTTGACGATCTTCGACCCTCTTTGGGCTGCTA |
| | | CGGCGACAAACTGGTTCGCAGCCCCAACATAGACCAGCTTGCT |
| | | TCCCATTCACTGCTTTTTCAGAACGCGTTTGCTCAGCAAGCCG |
| | | TCTGCGCACCATCCCGCGTTTCTTTTCTTACTGGACGACGCCC |
| | | TGACACGACCCGACTGTACGATTTTAATAGTTACTGGCGCGTT |
| | | CATGCCGGCAATTTCTCAACCATCCCTCAGTACTTCAAAGAGA |
| | | ACGGATACGTCACCATGAGCGTTGGCAAGGTGTTCCATCCAGG |
| | | CATCTCTTCCAACCATACCGACGATAGCCCATACAGCTGGTCC |
| | | TTTCCCCCATATCATCCCTCAAGTGAAAAATATGAAAATACAA |
| | | AGACATGCAGAGGTCCCGACGGCGAGCTTCACGCCAATCTCCT |
| | | GTGTCCAGTTGATGTGCTCGATGTGCCAGAGGGGACACTCCCT |
| | | GATAAACAATCTACTGAGCAGGCTATCCAGCTCCTTGAGAAAA |
| | | TGAAAACCTCTGCCAGCCCCTTTTTCTTGGCCGTCGGTTACCA |
| | | CAAGCCCCACATTCCATTCCGGTATCCAAAAGAATTCCAGAAA |
| | | TTGTATCCTCTTGAAAACATCACCCTGGCCCCCGACCCTGAAG |
| | | TGCCCGATGGCCTGCCCCCTGTCGCCTATAACCCATGGATGGA |
| | | TATCAGGCAGAGAGAGGACGTGCAGGCCCTTAATATCTCAGTT |
| | | CCCTACGGACCAATTCCCGTTGATTTTCAAAGAAAGATCCGCC |
| | | AGTCCTACTTTGCTAGCGTCTCATACCTCGACACACAGGTCGG |
| | | CAGACTTCTCAGCGCCCTCGACGACCTGCAATTGGCTAACAGC |
| | | ACCATCATTGCCTTCACCTCTGACCACGGGTGGGCGCTCGGCG |
| | | AACACGGCGAGTGGGCCAAATATTCAAATTTCGACGTCGCCAC |
| | | ACACGTACCCCTTATCTTTTACGTCCCCGGTAGAACCGCTAGT |
| | | CTGCCCGAAGCAGGAGAGAAACTGTTCCCCTATCTGGACCCCT |
| | | TTGATTCAGCTAGCCAATTGATGGAGCCCGGTAGACAATCCAT |
| | | GGATTTGGTTGAACTCGTGTCCCTCTTTCCCACGCTGGCCGGT |
| | | CTGGCCGGTCTCCAAGTTCCCCCCAGGTGCCCCGTTCCTTCTT |
| | | TCCACGTAGAGCTGTGCAGGGAGGGAAAAAACTTGCTTAAACA |
| | | TTTTCGGTTTCGCGACCTGGAGGAAGACCCCTACTTGCCCGGT |
| | | AATCCCCGCGAGCTGATCGCTTATTCCCAATACCCTAGACCTA |
| | | GCGACATCCCTCAGTGGAATTCCGATAAGCCGTCCCTCAAGGA |
| | | CATTAAGATTATGGGATACTCTATTCGCACTATTGACTACAGA |
| | | TATACCGTCTGGGTGGGCTTCAATCCTGATGAATTCCTGGCAA |
| | | ACTTTTCCGATATTCACGCTGGTGAGCTGTATTTCGTCGACTC |
| | | CGATCCACTGCAAGACCACAATATGTACAACGATTCCCAAGGC |
| | | GGAGATTTGTTCCAGCTCTTGATGCCTTGA |
| 185 | IDS Transgene | AGCGAAACCCAGGCCAACTCAACTACAGATGCGCTTAACGTCC |
| | | TGCTCATCATCGTGGACGATTTGCGGCCGTCGCTTGGCTGCTA |
| | | TGGAGATAAGCTCGTCCGCTCGCCGAACATCGATCAGTTGGCC |
| | | TCACACTCACTGCTTTTCCAAAATGCGTTTGCGCAGCAGGCTG |
| | | TCTGTGCACCTTCAAGAGTCTCATTCTTGACCGGGCGACGCCC |
| | | TGACACAACGCGGCTGTACGACTTCAACAGCTACTGGAGAGTC |
| | | CACGCGGGTAACTTTTCAACTATCCCACAGTACTTTAAAGAGA |
| | | ACGGATACGTGACAATGAGCGTGGGAAAGGTCTTTCACCCCGG |
| | | CATCTCCTCGAATCACACCGACGATTCGCCCTACTCGTGGTCG |
| | | TTTCCTCCCTACCATCCTTCGAGCGAGAAGTATGAGAACACGA |
| | | AAACTTGTCGCGGACCCGACGGAGAGCTGCACGCTAATCTGCT |
| | | GTGTCCGGTGGATGTCTTGGACGTGCCCGAGGGAACGCTCCCC |
| | | GACAAGCAGTCAACGGAGCAGGCGATTCAGTTGCTGGAGAAGA |
| | | TGAAAACAAGCGCGTCGCCTTTCTTCCTCGCCGTGGGGTATCA |
| | | CAAGCCCCATATTCCTTTCCGCTACCCGAAGGAGTTCCAGAAA |
| | | CTTTATCCTTTGGAAAACATCACTTTGGCACCGGACCCGGAAG |
| | | TCCCCGACGGTCTGCCACCCGTGGCCTACAATCCCTGGATGGA |
| | | TATCAGGCAGAGGGAAGATGTGCAGGCACTCAACATCTCAGTC |
| | | CCCTACGGGCCTATTCCAGTCGATTTTCAACGCAAGATTCGGC |
| | | AGTCGTATTTTGCGTCGGTGTCCTACCTCGATACGCAAGTAGG |
| | | TCGACTTCTGAGCGCGCTTGATGACCTTCAGCTGGCAAATTCC |
| | | ACAATCATCGCCTTTACGTCGGACCATGGGTGGGCGTTGGGAG |
| | | AGCATGGAGAGTGGGCAAAGTATAGCAATTTTGATGTAGCAAC |
| | | GCACGTGCCCCTGATTTTCTACGTGCCGGGTAGAACGGCCTCG |
| | | CTTCCCGAGGCAGGCGAAAAACTTTTTCCCTATCTCGATCCAT |
| | | TCGACTCGGCGAGCCAGCTTATGGAACCGGGCAGACAATCCAT |
| | | GGACTTGGTAGAATTGGTGTCCCTTTTTCCGACCCTCGCCGGG |
| | | TTGGCGGGCTTGCAAGTACCCCCTAGATGCCCTGTACCGAGCT |
| | | TCCATGTGGAACTCTGCCGCGAAGGGAAAAACCTCCTCAAACA |
| | | CTTTCGGTTCAGGGACCTTGAGGAGGACCCCTATCTGCCAGGG |
| | | AATCCGCGAGAGTTGATTGCCTATTCCCAGTATCCGCGACCCA |
| | | GCGATATTCCTCAATGGAACTCCGATAAGCCCTCCCTCAAAGA |
| | | CATCAAGATTATGGGGTACTCGATGAGGACCATCGACTATCGC |
| | | TACACAGTGTGGGTAGGGTTCAATCCTGACGAATTCCTCGCGA |
| | | ACTTTTCGGACATCCACGCTGGTGAGCTGTATTTCGTAGACTC |
| | | GGACCCGTTGCAAGATCACAATATGTATAATGATTCCCAAGGA |
| | | GGAGATTTGTTCCAGCTGCTCATGCCGTGA |
| 186 | IDS Transgene | TCAGAGACTCAAGCAAATAGCACTACGGACGCCTTGAATGTTT |
| | | TGCTGATTATAGTGGATGACCTCAGACCTTCACTCGGCTGTTA |
| | | CGGTGACAAACTGGTCCGCTCTCCGAATATCGACCAACTGGCA |
| | | AGCCACTCCCTCCTTTTCCAAAACGCATTCGCTCAACAAGCAG |
| | | TTTGTGCCCCCAGTAGAGTGTCCTTCTTGACTGGTCGCAGGCC |
| | | CGACACCACCCGCCTGTACGATTTTAACTCATATTGGCGCGTT |
| | | CATGCCGGCAACTTTTCTACAATACCACAATACTTTAAGGAAA |
| | | ATGGCTACGTAACTATGAGTGTGGGCAAGGTGTTTCACCCCGG |
| | | TATTTCAAGCAATCACACAGACGACTCTCCCTACTCCTGGTCC |
| | | TTTCCCCCATACCATCCTTCCTCAGAGAAGTACGAAAATACCA |
| | | AGACGTGTAGAGGTCCGGACGGCGAACTGCACGCAAACCTGTT |
| | | GTGCCCTGTTGACGTACTCGACGTCCCGGAAGGCACCCTCCCC |
| | | GACAAGCAATCTACCGAGCAGGCCATTCAGCTCCTCGAAAAGA |
| | | TGAAAACAAGTGCATCCCCCTTTTTCCTGGCTGTAGGTTATCA |
| | | TAAACCCCACATTCCATTCCGGTATCCTAAAGAATTTCAGAAG |
| | | CTGTACCCCCTTGAAAACATTAGACTGGCACCAGACCCAGAAG |
| | | TCCCAGACGGACTCCCCCCAGTGGCCTATAACCCATGGATGGA |
| | | CATCAGGCAGCGCGAAGACGTGCAGGCTCTTAACATCAGCGTC |
| | | CCATATGGCCCAATACCTGTCGACTTTCAACGCAAGATTAGAC |
| | | AATCCTATTTCGCTTCTGTGAGTTACCTGGACACACAAGTAGG |
| | | AAGACTGCTCAGCGCCCTTGACGATCTGCAACTCGCTAATTCT |
| | | ACCATAATTGCCTTTACCAGCGACCATGGATGGGCACTCGGAG |
| | | AACACGGCGAATGGGCAAAGTACTCCAATTTCGATGTCGCAAC |
| | | CCACGTTCCCTTGATATTCTATGTCCCCGGCCGCACTGCGTCC |
| | | TTGCCAGAAGCTGGGGAAAAACTCTTTCCATATCTGGACCCCT |
| | | TCGACTCTGCATCCCAACTGATGGAACCCGGTAGACAAAGTAT |
| | | GGATCTGGTCGAGCTCGTTTCACTCTTTCCGACCCTTGCCGGT |
| | | CTCGCCGGCCTTCAGGTGCCACCACGATGCCCCGTTCCGAGCT |
| | | TCCACGTCGAGCTTTGTAGAGAAGGGAAAAACCTCCTGAAACA |
| | | TTTCCGATTTCGCGACCTGGAGGAAGACCCATACCTGCCCGGG |
| | | AATCCTAGAGAACTCATCGCATATTCTCAGTACCCCAGACCCT |
| | | CCGACATCCCACAGTGGAACTCTGACAAACCATCTTTGAAAGA |
| | | CATTAAGATTATGGGCTACAGCATCCGGACTATAGATTACAGG |
| | | TATACCGTATGGGTTGGATTCAATCCCGATGAATTCCTCGCGA |
| | | ATTTCTCAGACATCCACGCAGGAGAACTCTATTTCGTGGACTC |
| | | AGACCCCCTTCAAGATCACAACATGTAGAACGATTCCCAAGGA |
| | | GGTGATCTTTTTCAGTTGCTCATGCCTTGA |
| 187 | IDS Transgene | AGTGAAACGCAGGCGAACTCAACCACCGATGCGCTGAACGTTC |
| | | TGCTTATTATCGTGGATGATCTGCGACCCTCACTTGGTTGCTA |
| | | TGGCGATAAATTGGTTAGAAGTCCGAACATAGACCAGCTGGCG |
| | | AGTCATTCTCTCCTCTTCCAAAACGCGTTCGCACAACAGGCCG |
| | | TTTGCGCCCCTTCAAGAGTATCCTTTCTGACAGGCAGACGCCC |
| | | CGATACTACTAGGCTGTATGACTTCAATTCCTACTGGCGCGTG |
| | | CACGCAGGTAATTTCTCTAGAATCCCCCAGTACTTCAAAGAAA |
| | | ACGGATACGTTACCATGAGCGTCGGCAAAGTGTTCCATCCCGG |
| | | AATTTCTAGCAACCATACGGATGACAGCCCCTATTCCTGGTCA |
| | | TTTCCACCGTAGCATCCTTCGAGTGAAAAATATGAGAACACTA |
| | | AAACTTGTCGCGGACCTGACGGAGAATTGCACGCAAACCTTCT |
| | | CTGCCCCGTAGATGTGCTCGATGTGCCTGAAGGAACTCTCCCA |
| | | GACAAGCAGAGTACCGAACAAGCCATTCAGCTGCTGGAAAAGA |
| | | TGAAAACGTCCGCCTCACCTTTCTTCCTCGCAGTCGGTTACCA |
| | | CAAGCCCCACATTCCTTTTAGATACCCTAAAGAGTTTCAGAAA |
| | | CTGTATCCCCTTGAAAATATCACCCTCGCTCCCGACCCCGAGG |
| | | TCCCGGACGGCCTGCCCCCTGTTGCATACAACCCCTGGATGGA |
| | | TATCAGACAACGGGAGGATGTTCAAGCACTCAACATCTCAGTA |
| | | CCATACGGCCCAATCCCTGTCGATTTCCAAAGGAAAATCAGGC |
| | | AGTCCTACTTTGCAAGCGTGTCTTATCTCGACACCCAGGTCGG |
| | | AAGACTGCTGTCCGCCCTCGACGACCTTCAATTGGCTAACTCT |
| | | ACAATCATTGCCTTCACTAGCGATCACGGGTGGGCGCTTGGCG |
| | | AGCACGGAGAATGGGCCAAATACTCTAATTTTGATGTTGCCAC |
| | | CCACGTGCCCCTCATATTTTATGTTCCAGGTAGAACCGCAAGC |
| | | CTGCCAGAAGCCGGTGAGAAGCTGTTTCCTTACCTCGATCCTT |
| | | TCGATAGTGCATCCCAACTGATGGAGCCAGGTCGACAATCTAT |
| | | GGACCTGGTAGAGCTGGTCTCTCTGTTCCCAACGCTCGCCGGA |
| | | CTTGCTGGACTGCAGGTGCCACCCCGCTGCCCTGTACCCTCCT |
| | | TCCACGTTGAGCTCTGCCGCGAAGGCAAGAACCTGTTGAAACA |
| | | TTTTCGATTCAGAGACCTTGAAGAGGACCCATACCTCCCAGGA |
| | | AATCCAAGAGAGCTGATTGCTTATTCTCAATATCCCAGGCCCA |
| | | GTGACATACCACAGTGGAATAGCGATAAACCCTCACTTAAAGA |
| | | CATTAAGATAATGGGCTATTCCATCCGGACAATTGATTACAGA |
| | | TACACAGTTTGGGTGGGGTTTAACCCAGACGAATTCCTTGCGA |
| | | ATTTCAGCGATATTCATGCCGGAGAACTTTATTTTGTTGATAG |
| | | CGACCCCCTCCAGGACCACAACATGTACAACGACTCACAGGGT |
| | | GGCGATCTCTTTCAGCTCCTGATGCCGTGA |
| 188 | IDS Transgene | TCTGAAACCCAGGCTAACTCTACGACCGACGCATTGAATGTTC |
| | | TGCTTATCATTGTAGATGACCTTCGCCCCAGTTTGGGATGTTA |
| | | TGGCGATAAGCTGGTGCGCTCACCTAATATTGATCAGTTGGCA |
| | | AGCCATAGCCTTTTGTTCCAAAATGCTTTTGCTCAGCAGGCTG |
| | | TATGTGCACCGAGTAGAGTTTCCTTCCTCACCGGACGCAGACC |
| | | AGACACCACAAGACTCTACGACTTTAACTCATACTGGAGGGTC |
| | | CACGCTGGGAATTTGAGTACGATCCCGCAGTATTTCAAAGAAA |
| | | ATGGCTACGTTACCATGTCCGTCGGCAAGGTGTTTCACCCCGG |
| | | CATCTCATCAAATCATACAGACGATAGCCCTTATTCTTGGTCT |
| | | TTCCCTCCTTATCATCCATCCAGCGAAAAATACGAGAACACTA |
| | | AAACATGTAGAGGTCCAGATGGAGAGCTGCACGCCAACCTGCT |
| | | GTGCCCTGTGGATGTTCTCGACGTACCTGAAGGCACCCTTCCA |
| | | GACAAACAGAGCACCGAACAGGCCATCCAGCTTCTGGAGAAGA |
| | | TGAAGACCAGCGCCTCACCTTTCTTCCTCGCCGTAGGCTACCA |
| | | CAAACCGCACATCCCCTTTAGATACCCAAAGGAATTTCAGAAG |
| | | CTGTACCCCCTGGAAAATATAACATTGGCTCCAGACCCGGAAG |
| | | TGCCCGATGGGTTGCCCCCCGTAGCCTATAATCCTTGGATGGA |
| | | TATTAGACAACGGGAAGACGTCCAGGCCCTCAATATTTCTGTC |
| | | CCTTACGGACCAATCCCTGTTGATTTTCAGAGAAAGATAAGAC |
| | | AGTCCTATTTTGCAAGTGTATCCTACCTTGACACCCAGGTCGG |
| | | CCGGCTGTTGTCTGCTCTGGACGACCTGCAACTCGCTAACAGT |
| | | ACAATCATAGCCTTTACTAGCGACCACGGATGGGCTCTGGGAG |
| | | AACATGGAGAATGGGCCAAGTATTCTAACTTCGATGTCGCCAC |
| | | ACACGTCCCACTCATATTTTACGTTCCTGGTCGAACCGCTAGC |
| | | CTGCCTGAAGCCGGAGAAAAGCTGTTTCCTTATCTCGACCCTT |
| | | TCGATTCCGCAAGCCAGTTGATGGAACCCGGCCGGCAATCAAT |
| | | GGATCTCGTGGAACTGGTGTCACTTTTTCCTACACTCGCTGGA |
| | | CTCGCTGGCCTTCAAGTCCCTCCCCGATGTCCTGTCCCATCAT |
| | | TTCACGTAGAGCTGTGTAGAGAAGGGAAGAATCTGCTGAAACA |
| | | CTTCCGGTTCCGGGATCTTGAAGAAGATCCATATCTCCCAGGC |
| | | AACCCTCGCGAACTCATCGCTTATAGCCAGTATCCTCGGCCCA |
| | | GTGACATACCCCAGTGGAATTCCGACAAACCATCACTTAAAGA |
| | | TATCAAAATTATGGGATACTCCATTCGAACCATAGACTATAGG |
| | | TACACCGTGTGGGTTGGCTTTAATCCAGATGAGTTTTTGGCAA |
| | | ACTTTTCAGACATTCACGCCGGAGAGCTGTATTTTGTGGACAG |
| | | TGACCCTCTGCAGGACCATAATATGTAGAACGATTCACAAGGC |
| | | GGCGACCTCTTCCAACTGCTGATGCCCTGA |
| 189 | IDS Transgene | TCACGGCATGAGCAGCTGGAACAAATCTCCTCCTTGGGAATCA |
| | (reverse | TTATACATATTGTGATCTTGCAACGGGTCCGAGTCTACGAAAT |
| | complement) | ACAGCTCACCAGCGTGGATGTCCGAAAAGTTCGCGAGGAATTC |
| | | GTCAGGATTGAACCCTACCCACACTGTGTAGCGATAGTCGATG |
| | | GTCCTGATCGAGTACCCCATAATCTTGATGTCTTTGAGGGAGG |
| | | GCTTATCGGAGTTCCATTGAGGAATATCGCTGGGTCGCGGATA |
| | | CTGGGAATAGGCAATCAACTCTCGCGGATTCCCTGGCAGATAG |
| | | GGGTCCTCCTCAAGGTCCCTGAACCGAAAGTGTTTGAGGAGGT |
| | | TTTTCCCTTCGCGGCAGAGTTCCACATGGAAGCTCGGTACAGG |
| | | GCATCTAGGGGGTACTTGCAAGCCCGCCAACCCGGCGAGGGTC |
| | | GGAAAAAGGGACACCAATTCTACCAAGTCCATGGATTGTCTGC |
| | | CCGGTTCCATAAGCTGGCTCGCCGAGTCGAATGGATCGAGATA |
| | | GGGAAAAAGTTTTTCGCCTGCCTCGGGAAGCGAGGCCGTTCTA |
| | | CCCGGCACGTAGAAAATCAGGGGCACGTGCGTTGCTACATCAA |
| | | AATTGCTATACTTTGCCCACTCTCCATGCTCTCCCAACGCCCA |
| | | CCCATGGTCCGACGTAAAGGCGATGATTGTGGAATTTGCCAGC |
| | | TGAAGGTCATCAAGCGCGCTCAGAAGTCGACCTACTTGCGTAT |
| | | CGAGGTAGGACACCGACGCAAAATACGACTGCCGAATCTTGCG |
| | | TTGAAAATCGACTGGAATAGGCCCGTAGGGGACTGAGATGTTG |
| | | AGTGCCTGCACATCTTCCCTCTGCCTGATATCCATCCAGGGAT |
| | | TGTAGGCCACGGGTGGCAGACCGTCGGGGACTTCCGGGTCCGG |
| | | TGCCAAAGTGATGTTTTCCAAAGGATAAAGTTTCTGGAACTCC |
| | | TTCGGGTAGCGGAAAGGAATATGGGGCTTGTGATACCCCACGG |
| | | CGAGGAAGAAAGGCGACGCGCTTGTTTTCATCTTCTCCAGCAA |
| | | CTGAATCGCCTGCTCCGTTGACTGCTTGTCGGGGAGCGTTCCC |
| | | TCGGGCACGTCCAAGACATCCACCGGACACAGCAGATTAGCGT |
| | | GCAGCTCTCCGTCGGGTCCGCGACAAGTTTTCGTGTTCTCATA |
| | | CTTCTCGCTCGAAGGATGGTAGGGAGGAAACGACCACGAGTAG |
| | | GGCGAATCGTCGGTGTGATTCGAGGAGATGCCGGGGTGAAAGA |
| | | CCTTTCCCACGCTCATTGTCACGTATCCGTTCTCTTTAAAGTA |
| | | CTGTGGGATAGTTGAAAAGTTACCCGCGTGGACTCTCCAGTAG |
| | | CTGTTGAAGTCGTACAGCCGCGTTGTGTCAGGGCGTCGCCCGG |
| | | TCAAGAATGAGACTCTTGAAGGTGCACAGACAGCCTGCTGCGC |
| | | AAACGCATTTTGGAAAAGCAGTGAGTGTGAGGCCAACTGATCG |
| | | ATGTTCGGCGAGCGGACGAGCTTATCTCCATAGCAGCCAAGCG |
| | | ACGGCCGCAAATCGTCCACGATGATGAGCAGGACGTTAAGCGC |
| | | ATCTGTAGTTGAGTTGGCCTGGGTTTCGCT |
| 190 | IDS Transgene | TCAAGGCATCAAGAGCTGGAACAAATCTCCGCCTTGGGAATCG |
| | (reverse | TTGTACATATTGTGGTCTTGCAGTGGATCGGAGTCGACGAAAT |
| | complement) | ACAGCTCACCAGCGTGAATATCGGAAAAGTTTGCCAGGAATTC |
| | | ATGAGGATTGAAGCCCACCCAGACGGTATATCTGTAGTCAATA |
| | | GTGCGAATAGAGTATCCCATAATCTTAATGTCCTTGAGGGACG |
| | | GCTTATCGGAATTCCACTGAGGGATGTCGCTAGGTCTAGGGTA |
| | | TTGGGAATAAGCGATCAGCTCGCGGGGATTACCGGGCAAGTAG |
| | | GGGTCTTCCTCCAGGTCGCGAAACCGAAAATGTTTAAGCAAGT |
| | | TTTTTCCCTCCCTGCACAGCTCTACGTGGAAAGAAGGAACGGG |
| | | GCACCTGGGGGGAACTTGGAGACCGGCCAGACCGGCCAGCGTG |
| | | GGAAAGAGGGACACGAGTTCAACCAAATCCATGGATTGTCTAC |
| | | CGGGCTCCATCAATTGGCTAGCTGAATCAAAGGGGTCCAGATA |
| | | GGGGAACAGTTTCTCTCCTGCTTCGGGCAGACTAGCGGTTCTA |
| | | CCGGGGACGTAAAAGATAAGGGGTACGTGTGTGGCGACGTCGA |
| | | AATTTGAATATTTGGCCCACTCGCCGTGTTCGCCGAGCGCCCA |
| | | CCCGTGGTCAGAGGTGAAGGCAATGATGGTGCTGTTAGCCAAT |
| | | TGCAGGTCGTCGAGGGCGCTGAGAAGTCTGCCGACCTGTGTGT |
| | | CGAGGTATGAGACGCTAGCAAAGTAGGACTGGCGGATCTTTCT |
| | | TTGAAAATCAACGGGAATTGGTCCGTAGGGAACTGAGATATTA |
| | | AGGGCCTGCACGTCCTCTCTCTGCCTGATATCCATCCATGGGT |
| | | TATAGGCGACAGGGGGCAGGCCATCGGGCACTTCAGGGTCGGG |
| | | GGCCAGGGTGATGTTTTCAAGAGGATACAATTTCTGGAATTCT |
| | | TTTGGATACCGGAATGGAATGTGGGGCTTGTGGTAACCGACGG |
| | | CCAAGAAAAAGGGGCTGGCAGAGGTTTTCATTTTCTCAAGGAG |
| | | CTGGATAGCCTGCTCAGTAGATTGTTTATCAGGGAGTGTCCCC |
| | | TCTGGCACATCGAGCACATCAACTGGACACAGGAGATTGGCGT |
| | | GAAGCTCGCCGTCGGGACCTCTGCATGTCTTTGTATTTTCATA |
| | | TTTTTCACTTGAGGGATGATATGGGGGAAAGGACCAGCTGTAT |
| | | GGGCTATCGTCGGTATGGTTGGAAGAGATGCCTGGATGGAACA |
| | | CCTTGCCAACGCTCATGGTGACGTATCCGTTCTCTTTGAAGTA |
| | | CTGAGGGATGGTTGAGAAATTGCCGGCATGAACGCGCCAGTAA |
| | | CTATTAAAATCGTACAGTCGGGTCGTGTCAGGGCGTCGTCCAG |
| | | TAAGAAAAGAAACGCGGGATGGTGCGCAGACGGCTTGCTGAGC |
| | | AAACGCGTTCTGAAAAAGCAGTGAATGGGAAGCAAGCTGGTCT |
| | | ATGTTGGGGCTGCGAACCAGTTTGTCGCCGTAGCAGCCCAAAG |
| | | AGGGTCGAAGATCGTCAACAATGATAAGGAGGAGGTTCAATGC |
| | | ATCAGTGGTGGAGTTAGCCTGCGTCTCACT |
| 191 | IDS Transgene | TCAAGGCATGAGCAACTGAAAAAGATCACCTCCTTGGGAATCG |
| | (reverse | TTGTACATGTTGTGATCTTGAAGGGGGTCTGAGTCCACGAAAT |
| | complement) | AGAGTTCTCCTGCGTGGATGTCTGAGAAATTCGCGAGGAATTC |
| | | ATCGGGATTGAATCCAACCCATACGGTATACCTGTAATCTATA |
| | | GTCCGGATGCTGTAGCCCATAATCTTAATGTCTTTCAAAGATG |
| | | GTTTGTCAGAGTTCCACTGTGGGATGTCGGAGGGTCTGGGGTA |
| | | CTGAGAATATGCGATGAGTTCTCTAGGATTCCCGGGCAGGTAT |
| | | GGGTCTTCCTCCAGGTCGCGAAATCGGAAATGTTTCAGGAGGT |
| | | TTTTCCCTTCTCTACAAAGCTCGACGTGGAAGCTCGGAACGGG |
| | | GCATCGTGGTGGCACCTGAAGGCCGGCGAGACCGGCAAGGGTC |
| | | GGAAAGAGTGAAACGAGCTCGACCAGATCCATACTTTGTCTAC |
| | | CGGGTTCCATCAGTTGGGATGCAGAGTCGAAGGGGTCCAGATA |
| | | TGGAAAGAGTTTTTCCCCAGCTTCTGGCAAGGACGCAGTGCGG |
| | | CCGGGGACATAGAATATCAAGGGAACGTGGGTTGCGACATCGA |
| | | AATTGGAGTACTTTGCCCATTCGCCGTGTTCTCCGAGTGCCCA |
| | | TCCATGGTCGCTGGTAAAGGCAATTATGGTAGAATTAGCGAGT |
| | | TGCAGATCGTCAAGGGCGCTGAGCAGTCTTCCTACTTGTGTGT |
| | | CCAGGTAACTCACAGAAGCGAAATAGGATTGTCTAATCTTGCG |
| | | TTGAAAGTCGACAGGTATTGGGCCATATGGGACGCTGATGTTA |
| | | AGAGCCTGCACGTCTTCGCGCTGCCTGATGTCCATCCATGGGT |
| | | TATAGGCCACTGGGGGGAGTCCGTCTGGGACTTCTGGGTCTGG |
| | | TGCCAGTGTAATGTTTTCAAGGGGGTACAGCTTCTGAAATTCT |
| | | TTAGGATACCGGAATGGAATGTGGGGTTTATGATAACCTACAG |
| | | CCAGGAAAAAGGGGGATGCACTTGTTTTCATCTTTTCGAGGAG |
| | | CTGAATGGCCTGCTCGGTAGATTGCTTGTCGGGGAGGGTGCCT |
| | | TCCGGGACGTCGAGTACGTCAACAGGGCACAACAGGTTTGCGT |
| | | GCAGTTCGCCGTCCGGACCTCTACACGTCTTGGTATTTTCGTA |
| | | CTTCTCTGAGGAAGGATGGTATGGGGGAAAGGACCAGGAGTAG |
| | | GGAGAGTCGTCTGTGTGATTGCTTGAAATACCGGGGTGAAACA |
| | | CCTTGCCCACACTCATAGTTACGTAGCCATTTTCCTTAAAGTA |
| | | TTGTGGTATTGTAGAAAAGTTGCCGGCATGAACGCGCCAATAT |
| | | GAGTTAAAATCGTACAGGCGGGTGGTGTCGGGCCTGCGACCAG |
| | | TCAAGAAGGACACTCTACTGGGGGCACAAACTGCTTGTTGAGC |
| | | GAATGCGTTTTGGAAAAGGAGGGAGTGGCTTGCCAGTTGGTCG |
| | | ATATTCGGAGAGCGGACGAGTTTGTCACCGTAACAGCCGAGTG |
| | | AAGGTCTGAGGTCATCCACTATAATCAGCAAAACATTCAAGGC |
| | | GTCCGTAGTGCTATTTGCTTGAGTCTCTGA |
| 192 | IDS Transgene | TCACGGCATCAGGAGCTGAAAGAGATCGCCACCCTGTGAGTCG |
| | (reverse | TTGTACATGTTGTGGTCCTGGAGGGGGTCGCTATCAACAAAAT |
| | complement) | AAAGTTCTCCGGCATGAATATCGCTGAAATTCGCAAGGAATTC |
| | | GTCTGGGTTAAACCCCACCCAAACTGTGTATCTGTAATCAATT |
| | | GTCCGGATGGAATAGCCCATTATCTTAATGTCTTTAAGTGAGG |
| | | GTTTATCGCTATTCCACTGTGGTATGTCACTGGGCCTGGGATA |
| | | TTGAGAATAAGCAATCAGCTCTCTTGGATTTCCTGGGAGGTAT |
| | | GGGTCCTCTTCAAGGTCTCTGAATCGAAAATGTTTCAACAGGT |
| | | TCTTGCCTTCGCGGCAGAGCTCAACGTGGAAGGAGGGTACAGG |
| | | GCAGCGGGGTGGCACCTGCAGTCCAGCAAGTCCGGCGAGCGTT |
| | | GGGAACAGAGAGACCAGCTCTACCAGGTCCATAGATTGTCGAC |
| | | CTGGCTCCATCAGTTGGGATGCACTATCGAAAGGATCGAGGTA |
| | | AGGAAACAGCTTCTCACCGGCTTCTGGCAGGCTTGCGGTTCTA |
| | | CCTGGAACATAAAATATGAGGGGCACGTGGGTGGCAACATCAA |
| | | AATTAGAGTATTTGGCCCATTCTCCGTGCTCGCCAAGCGCCCA |
| | | CCCGTGATCGCTAGTGAAGGCAATGATTGTAGAGTTAGCCAAT |
| | | TGAAGGTCGTCGAGGGCGGACAGCAGTCTTCCGACCTGGGTGT |
| | | CGAGATAAGACACGCTTGCAAAGTAGGACTGCCTGATTTTCCT |
| | | TTGGAAATCGACAGGGATTGGGCCGTATGGTACTGAGATGTTG |
| | | AGTGCTTGAACATCCTCCCGTTGTCTGATATCCATCCAGGGGT |
| | | TGTATGCAACAGGGGGCAGGCCGTCCGGGACCTCGGGGTCGGG |
| | | AGCGAGGGTGATATTTTCAAGGGGATACAGTTTCTGAAACTCT |
| | | TTAGGGTATCTAAAAGGAATGTGGGGCTTGTGGTAACCGACTG |
| | | CGAGGAAGAAAGGTGAGGCGGACGTTTTCATCTTTTCCAGCAG |
| | | CTGAATGGCTTGTTCGGTACTCTGCTTGTCTGGGAGAGTTCCT |
| | | TCAGGCACATCGAGCACATCTACGGGGCAGAGAAGGTTTGCGT |
| | | GCAATTCTCCGTCAGGTCCGCGACAAGTTTTAGTGTTCTCATA |
| | | TTTTTCACTGGAAGGATGGTACGGTGGAAATGACCAGGAATAG |
| | | GGGCTGTCATCCGTATGGTTGCTAGAAATTCCGGGATGGAACA |
| | | CTTTGCCGACGCTCATGGTAACGTATCCGTTTTCTTTGAAGTA |
| | | CTGGGGGATTGTAGAGAAATTACCTGCGTGCACGCGCCAGTAG |
| | | GAATTGAAGTCATACAGCCTAGTAGTATCGGGGCGTCTGCCTG |
| | | TCAGAAAGGATACTCTTGAAGGGGCGCAAACGGCCTGTTGTGC |
| | | GAACGCGTTTTGGAAGAGGAGAGAATGACTCGCCAGCTGGTCT |
| | | ATGTTCGGAGTTCTAACCAATTTATCGCCATAGCAACCAAGTG |
| | | AGGGTCGCAGATCATCCACGATAATAAGCAGAACGTTCAGCGC |
| | | ATCGGTGGTTGAGTTCGCCTGCGTTTCACT |
| 193 | IDS Transgene | TCAGGGCATCAGCAGTTGGAAGAGGTCGCCGCCTTGTGAATCG |
| | (reverse | TTGTAGATATTATGGTCCTGCAGAGGGTCACTGTCCACAAAAT |
| | complement) | ACAGCTCTCCGGCGTGAATGTCTGAAAAGTTTGCCAAAAACTC |
| | | ATCTGGATTAAAGCCAACCCACACGGTGTACCTATAGTCTATG |
| | | GTTCGAATGGAGTATCCCATAATTTTGATATCTTTAAGTGATG |
| | | GTTTGTCGGAATTCCACTGGGGTATGTCACTGGGCCGAGGATA |
| | | CTGGCTATAAGCGATGAGTTCGCGAGGGTTGCCTGGGAGATAT |
| | | GGATCTTCTTCAAGATCCCGGAACCGGAAGTGTTTCAGCAGAT |
| | | TCTTCCCTTCTCTACACAGCTCTACGTGAAATGATGGGACAGG |
| | | ACATCGGGGAGGGACTTGAAGGCCAGCGAGTCCAGCGAGTGTA |
| | | GGAAAAAGTGACACCAGTTCCACGAGATCCATTGATTGCCGGC |
| | | CGGGTTCCATCAACTGGCTTGCGGAATCGAAAGGGTCGAGATA |
| | | AGGAAACAGCTTTTCTCCGGCTTCAGGCAGGCTAGCGGTTCGA |
| | | CCAGGAACGTAAAATATGAGTGGGACGTGTGTGGCGACATCGA |
| | | AGTTAGAATACTTGGCCCATTCTCCATGTTCTCCCAGAGCCCA |
| | | TCCGTGGTCGCTAGTAAAGGCTATGATTGTACTGTTAGCGAGT |
| | | TGCAGGTCGTCCAGAGCAGACAACAGCCGGCCGACCTGGGTGT |
| | | CAAGGTAGGATACACTTGCAAAATAGGACTGTCTTATCTTTCT |
| | | CTGAAAATCAACAGGGATTGGTCCGTAAGGGACAGAAATATTG |
| | | AGGGCCTGGACGTCTTCCCGTTGTCTAATATCCATCCAAGGAT |
| | | TATAGGCTACGGGGGGCAACCCATCGGGCACTTCCGGGTCTGG |
| | | AGCCAATGTTATATTTTCCAGGGGGTACAGCTTCTGAAATTCC |
| | | TTTGGGTATCTAAAGGGGATGTGCGGTTTGTGGTAGCCTACGG |
| | | CGAGGAAGAAAGGTGAGGCGCTGGTCTTCATCTTCTCCAGAAG |
| | | CTGGATGGCCTGTTCGGTGCTCTGTTTGTCTGGAAGGGTGCCT |
| | | TCAGGTACGTCGAGAACATCCACAGGGCACAGCAGGTTGGCGT |
| | | GCAGCTCTCCATCTGGACCTCTACATGTTTTAGTGTTCTCGTA |
| | | TTTTTCGCTGGATGGATGATAAGGAGGGAAAGACCAAGAATAA |
| | | GGGCTATCGTCTGTATGATTTGATGAGATGCCGGGGTGAAACA |
| | | CCTTGCCGACGGACATGGTAACGTAGCCATTTTCTTTGAAATA |
| | | CTGCGGGATCGTACTGAAATTCCCAGCGTGGACCCTCCAGTAT |
| | | GAGTTAAAGTCGTAGAGTCTTGTGGTGTCTGGTCTGCGTCCGG |
| | | TGAGGAAGGAAACTCTACTCGGTGCACATACAGCCTGCTGAGC |
| | | AAAAGCATTTTGGAACAAAAGGCTATGGCTTGCCAACTGATGA |
| | | ATATTAGGTGAGCGCACCAGCTTATCGCCATAACATCCCAAAC |
| | | TGGGGCGAAGGTCATCTACAATGATAAGCAGAACATTCAATGC |
| | | GTCGGTCGTAGAGTTAGCCTGGGTTTCAGA |

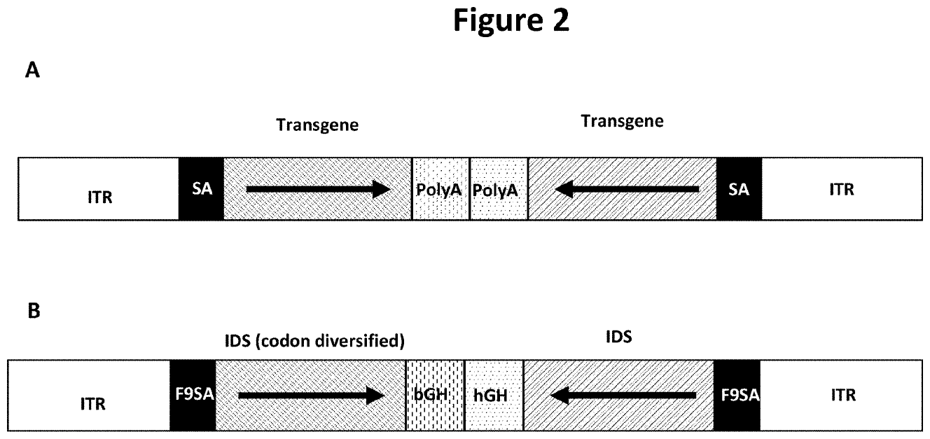

Non-limiting examples of 2-in-1 ZFN constructs include constructs as shown in FIG. 2; constructs comprising one or more of the sequences of Table 4 in any order or combination; and constructs as shown in Table 5.

TABLE 4

| SEQ | | |
|---|---|---|
| ID | Feature/ | |
| NO | Description | Amino Acid (aa) or Nucleic Acid (na) Sequence |
| 1 | FLAGtag (aa) | DYKDDDK |
| 2 | 3xFLAG(aa) | DYKDHDG-DYKDHDI-DYKDDDDK |
| 3 | NLS from the | PKKKRKV |
| | SV40 virus | |
| | large T gene | |
| | protein (aa) | |
| 4 | NLS from c- | PAAKRVKLD |
| | myc protein | |
| | (aa) | |
| 5 | NLS from | EGAPPAKRAR |
| | hepatitis delta | |
| | virus (aa) | |
| 6 | NLS from | VSRKRPRP |
| | polyoma T | |
| | protein (aa) | |
| 7 | NLS derived | KRPAATKKAGQAKKKKLD |
| | from the | |
| | nucleoplasmin | |
| | carboxy tail (aa) | |
| 8 | NLS described | NQSSNFGPMKGGNFGGRSSGPYGGGGQYFAKPRNQGGY |
| | by Siomi and | |
| | Dreyfuss (aa) | |
| 9 | NLS from the | PKTRRRPRRSQRKRPPT |
| | Rex protein in | |
| | HTLV-1 (aa) | |
| 156 | NLS (aa) | PKKKRKVGIH |
| 130 | Left ZFN | AAMAERPFQCRICMQNFSQSGNLARHIRTHTGEKPFACDICGRKFAL |
| | (ZFN-L)(aa) | KQNLCMHTKIHTGEKPFQCRICMQKFAWQSNLQNHTKIHTGEKPFQC |
| | | RICMRNFSTSGNLTRHIRTHTGEKPFACDICGRKFARRSHLTSHTKI |
| | | HLRGSQLVKSELEEKKSELRHKLKYVPHEYIELIEIARNSTQDRILE |
| | | MKVMEFFMKVYGYRGKHLGGSRKPDGAIYTVGSPIDYGVIVDTKAYS |
| | | GGYNLPIGQADEMERYVEENQTRDKHLNPNEWWKVYPSSVTEFKELF |
| | | VSGHFKGNYKAQLTRLNHITNCDGAVLSVEELLIGGEMIKAGTLTLE |
| | | EVRRKFNNGEINFRS |
| 131 | Right ZFN | AAMAERPFQCRICMRNFSQSSDLSRHIRTHTGEKPFACDICGRKFAL |
| | (ZFN-R)(aa) | KHNLLTHTKIHTGEKPFQCRICMQNFSDQSNLRAHIRTHTGEKPFAC |
| | | DICGRKFARNFSLTMHTKIHTGERGFQCRICMRNFSLRHDLERHIRT |
| | | HTGEKPFACDICGRKFAHRSNLNKHTKIHLRGSQLVKSELEEKKSEL |
| | | RHKLKYVPHEYIELIEIARNSTQDRILEMKVMEFFMKVYGYRGKHLG |
| | | GSRKPDGAIYTVGSPIDYGVIVDTKAYSGGYNLSIGQADEMQRYVKE |
| | | NQTRNKHINPNEWWKVYPSSVTEFKFLFVSGHFKGNYKAQLTRLNRK |
| | | TNCNGAVLSVEELLIGGEMIKAGTLTLEEVRRKFNNGEINF |
| 132 | Right ZFN- | DYKDHDGDYKDHDIDYKDDDDKMAPKKKRKVGIHGVPAAMAERPFQC |
| | T2A-Left ZFN | RICMRNFSQSSDLSRHIRTHTGEKPFACDICGRKFALKHNLLTHTKI |
| | with N-terminal | HTGEKPFQCRICMQNFSDQSNLRAHIRTHTGEKPFACDICGRKFARN |
| | modifications | FSLTMHTKIHTGERGFQCRICMRNFSLRHDLERHIRTHTGEKPFACD |
| | (comprising | ICGRKFAHRSNLNKHTKIHLRGSQLVKSELEEKKSELRHKLKYVPHE |
| | 3xFLAG, NLS, | YIELIEIARNSTQDRILEMKVMEFFMKVYGYRGKHLGGSRKPDGAIY |
| | ZFP-R, FokI, | TVGSPIDYGVIVDTKAYSGGYNLSIGQADEMQRYVKENQTRNKHINP |
| | T2A, 3xFLAG, | NEWWKVYPSSVTEFKFLFVSGHFKGNYKAQLTRLNRKTNCNGAVLSV |
| | NLS, ZFP-L, | EELLIGGEMIKAGTLTLEEVRRKFNNGEINFGSGEGRGSLLTCGDVE |
| | and FokI)(aa) | ENPGPTRAMDYKDHDGDYKDHDIDYKDDDDKMAPKKKRKVGIHGVPA |
| | | AMAERPFQCRICMQNFSQSGNLARHIRTHTGEKPFACDICGRKFALK |
| | | QNLCMHTKIHTGEKPFQCRICMQKFAWQSNLQNHTKIHTGEKPFQCR |
| | | ICMRNFSTSGNLTRHIRTHTGEKPFACDICGRKFARRSHLTSHTKIH |
| | | LRGSQLVKSELEEKKSELRHKLKYVPHEYIELIEIARNSTQDRILEM |
| | | KVMEFFMKVYGYRGKHLGGSRKPDGAIYTVGSPIDYGVIVDTKAYSG |
| | | GYNLPIGQADEMERYVEENQTRDKHLNPNEWWKVYPSSVTEFKFLFV |
| | | SGHFKGNYKAQLTRLNHITNCDGAVLSVEELLIGGEMIKAGTLTLEE |
| | | VRRKFNNGEINFRS- |
| 133 | Left ZFN-T2A- | DYKDHDGDYKDHDIDYKDDDDKMAPKKKRKVGIHGVPAAMAERPFQC |
| | Right ZFN | RICMQNFSQSGNLARHIRTHTGEKPFACDICGRKFALKQNLCMHTKI |
| | with N-terminal | HTGEKPFQCRICMQKFAWQSNLQNHTKIHTGEKPFQCRICMRNFSTS |
| | modifications | GNLTRHIRTHTGEKPFACDICGRKFARRSHLTSHTKIHLRGSQLVKS |
| | (comprising | ELEEKKSELRHKLKYVPHEYIELIEIARNSTQDRILEMKVMEFFMKV |
| | 3xFLAG, NLS, | YGYRGKHLGGSRKPDGAIYTVGSPIDYGVIVDTKAYSGGYNLPIGQA |
| | ZFP-L, FokI, | DEMERYVEENQTRDKHLNPNEWWKVYPSSVTEFKFLFVSGHFKGNYK |
| | T2A, 3xFLAG, | AQLTRLNHITNCDGAVLSVEELLIGGEMIKAGTLTLEEVRRKFNNGE |
| | NLS, ZFP-R, | INFRSGSGEGRGSLLTCGDVEENPGPTRAMDYKDHDGDYKDHDIDYK |
| | and FokI)(aa) | DDDDKMAPKKKRKVGIHGVPAAMAERPFQCRICMRNFSQSSDLSRHI |
| | | RTHTGEKPFACDICGRKFALKHNLLTHTKIHTGEKPFQCRICMQNFS |
| | | DQSNLRAHIRTHTGEKPFACDICGRKFARNFSLTMHTKIHTGERGFQ |
| | | CRICMRNFSLRHDLERHIRTHTGEKPFACDICGRKFAHRSNLNKHTK |
| | | IHLRGSQLVKSELEEKKSELRHKLKYVPHEYIELIEIARNSTQDRIL |
| | | EMKVMEFFMKVYGYRGKHLGGSRKPDGAIYTVGSPIDYGVIVDTKAY |
| | | SGGYNLSIGQADEMQRYVKENQTRNKHINPNEWWKVYPSSVTEFKFL |
| | | FVSGHFKGNYKAQLTRLNRKTNCNGAVLSVEELLIGGEMIKAGTLTL |
| | | EEVRRKFNNGEINF |
| 134 | Right ZFN- | AAMAERPFQCRICMRNFSQSSDLSRHIRTHTGEKPFACDICGRKFAL |
| | T2A-Left ZFN | KHNLLTHTKIHTGEKPFQCRICMQNFSDQSNLRAHIRTHTGEKPFAC |
| | (aa) | DICGRKFARNFSLTMHTKIHTGERGFQCRICMRNFSLRHDLERHIRT |
| | | HTGEKPFACDICGRKFAHRSNLNKHTKIHLRGSQLVKSELEEKKSEL |
| | | RHKLKYVPHEYIELIEIARNSTQDRILEMKVMEFFMKVYGYRGKHLG |
| | | GSRKPDGAIYTVGSPIDYGVIVDTKAYSGGYNLSIGQADEMQRYVKE |
| | | NQTRNKHINPNEWWKVYPSSVTEFKFLFVSGHFKGNYKAQLTRLNRK |
| | | TNCNGAVLSVEELLIGGEMIKAGTLTLEEVRRKFNNGEINFGSGEGR |
| | | GSLLTCGDVEENPGPAAMAERPFQCRICMQNFSQSGNLARHIRTHTG |
| | | EKPFACDICGRKFALKQNLCMHTKIHTGEKPFQCRICMQKFAWQSNL |
| | | QNHTKIHTGEKPFQCRICMRNFSTSGNLTRHIRTHTGEKPFACDICG |
| | | RKFARRSHLTSHTKIHLRGSQLVKSELEEKKSELRHKLKYVPHEYIE |
| | | LIEIARNSTQDRILEMKVMEFFMKVYGYRGKHLGGSRKPDGAIYTVG |
| | | SPIDYGVIVDTKAYSGGYNLPIGQADEMERYVEENQTRDKHLNPNEW |
| | | WKVYPSSVTEFKFLFVSGHFKGNYKAQLTRLNHITNCDGAVLSVEEL |
| | | LIGGEMIKAGTLTLEEVRRKFNNGEINFRS |
| 135 | Left ZFN-T2A- | AAMAERPFQCRICMQNFSQSGNLARHIRTHTGEKPFACDICGRKFAL |
| | Right ZFN (aa) | KQNLCMHTKIHTGEKPFQCRICMQKFAWQSNLQNHTKIHTGEKPFQC |
| | | RICMRNFSTSGNLTRHIRTHTGEKPFACDICGRKFARRSHLTSHTKI |
| | | HLRGSQLVKSELEEKKSELRHKLKYVPHEYIELIEIARNSTQDRILE |
| | | MKVMEFFMKVYGYRGKHLGGSRKPDGAIYTVGSPIDYGVIVDTKAYS |
| | | GGYNLPIGQADEMERYVEENQTRDKHLNPNEWWKVYPSSVTEFKFLF |
| | | VSGHFKGNYKAQLTRLNHITNCDGAVLSVEELLIGGEMIKAGTLTLE |
| | | EVRRKFNNGEINFRSGSGEGRGSLLTCGDVEENPGPVPAAMAERPFQ |
| | | CRICMRNFSQSSDLSRHIRTHTGEKPFACDICGRKFALKHNLLTHTK |
| | | IHTGEKPFQCRICMQNFSDQSNLRAHIRTHTGEKPFACDICGRKFAR |
| | | NFSLTMHTKIHTGERGFQCRICMRNFSLRHDLERHIRTHTGEKPFAC |
| | | DICGRKFAHRSNLNKHTKIHLRGSQLVKSELEEKKSELRHKLKYVPH |
| | | EYIELIEIARNSTQDRILEMKVMEFFMKVYGYRGKHLGGSRKPDGAI |
| | | YTVGSPIDYGVIVDTKAYSGGYNLSIGQADEMQRYVKENQTRNKHIN |
| | | PNEWWKVYPSSVTEFKFLFVSGHFKGNYKAQLTRLNRKTNCNGAVLS |
| | | VEELLIGGEMIKAGTLTLEEVRRKFNNGEINF |
| 10 | 5′ ITR (na) | CTGCGCGCTCGCTCGCTCACTGAGGCCGCCCGGGCAAAGCCCGGGCG |
| | | TCGGGCGACCTTTGGTCGCCCGGCCTCAGTGAGCGAGCGAGCGCGCA |
| | | GAGAGGGAGTGGCCAACTCCATCACTAGGGGTTCCT |
| 11 | ApoE hepatic | AGGCTCAGAGGCACACAGGAGTTTCTGGGCTCACCCTGCCCCCTTCC |
| | control region | AACCCCTCAGTTCCCATCCTCCAGCAGCTGTTTGTGTGCTGCCTCTG |
| | (Enhancer)(na) | AAGTCCACACTGAACAAACTTCAGCCTACTCATGTCCCTAAAATGGG |
| | | CAAACATTGCAAGCAGCAAACAGCAAACACACAGCCCTCCCTGCCTG |
| | | CTGACCTTGGAGCTGGGGCAGAGGTCAGAGACCTCTCTGGGCCCATG |
| | | CCACCTCCAACATCCACTCGACCCCTTGGAATTTCGGTGGAGAGGAG |
| | | CAGAGGTTGTCCTGGCGTGGTTTAGGTAGTGTGAGAGGG |
| 12 | hAAT | GATCTTGCTACCAGTGGAACAGCCACTAAGGATTCTGCAGTGAGAGC |
| | (Promoter)(na) | AGAGGGCCAGCTAAGTGGTACTCTCCCAGAGACTGTCTGACTCACGC |
| | | CACCCCCTCCACCTTGGACACAGGACGCTGTGGTTTCTGAGCCAGGT |
| | | ACAATGACTCCTTTCGGTAAGTGCAGTGGAAGCTGTACACTGCCCAG |
| | | GCAAAGCGTCCGGGCAGCGTAGGCGGGCGACTCAGATCCCAGCCAGT |
| | | GGACTTAGCCCCTGTTTGCTCCTCCGATAACTGGGGTGACCTTGGTT |
| | | AATATTCACCAGCAGCCTCCCCCGTTGCCCCTCTGGATCCACTGCTT |
| | | AAATACGGACGAGGACAGGGCCCTGTCTCCTCAGCTTCAGGCACCAC |
| | | CACTGACCTGGGACAGT |
| 13 | 5′ UTR (na) | CTTGTTCTTTTTGCAGAAGCTCAGAATAAACGCTCAACTTTGGCAGA |
| | | T |
| 14 | Human β-globin / | GTAAGTATCAAGGTTACAAGACAGGTTTAAGGAGACCAATAGAAACT |
| | IgG chimeric | GGGCTTGTCGAGACAGAGAAGACTCTTGCGTTTCTGATAGGCACCTA |
| | intron (na) | TTGGTCTTACTGACATCCACTTTGCCTTTCTCTCCACAG |
| 15 | 3xFLAG (na) | GACTACAAAGACCATGACGGTGATTATAAAGATCATGACATCGATTA |
| | | CAAGGATGACGATGACAAG |
| 16 | 3xFLAG (na) | GATTATAAAGATCATGACGGGGACTATAAGGATCACGACATAGACTA |
| | | CAAAGACGATGATGACAAA |
| 153 | 3xFLAG (na) | GATTAGAAAGATCACGACGGAGATTACAAAGATCACGACATTGACTA |
| | | TAAGGACGACGACGATAAA |
| 154 | 3XFLAG (na) | GATTACAAAGACCACGACGGAGACTACAAGGACCATGATATTGACTA |
| | | CAAAGACGATGATGATAAG |
| 17 | Left ZFN | GACTACAAAGACCATGACGGTGATTATAAAGATCATGACATCGATTA |
| | with N-terminal | CAAGGATGACGATGACAAGATGGCCCCCAAGAAGAAGAGGAAGGTCG |
| | modifications | GCATTCATGGGGTACCCGCCGCTATGGCTGAGAGGCCCTTCCAGTGT |
| | (comprising | CGAATCTGCATGCAGAACTTCAGTCAGTCCGGCAACCTGGCCCGCCA |
| | 3xFLAG, NLS, | CATCCGCACCCACACCGGCGAGAAGCCTTTTGCCTGTGACATTTGTG |
| | ZFP-L, and | GGAGGAAATTTGCCCTGAAGCAGAACCTGTGTATGCATACCAAGATA |
| | FokI)(na) | CACACGGGCGAGAAGCCCTTCCAGTGTCGAATCTGCATGCAGAAGTT |
| | Not diversified | TGCCTGGCAGTCCAACCTGCAGAACCATACCAAGATACACACGGGCG |
| | | AGAAGCCCTTCCAGTGTCGAATCTGCATGCGTAACTTCAGTACCTCC |
| | | GGCAACCTGACCCGCCACATCCGCACCCACACCGGCGAGAAGCCTTT |
| | | TGCCTGTGACATTTGTGGGAGGAAATTTGCCCGCCGCTCCCACCTGA |
| | | CCTCCCATACCAAGATACACCTGCGGGGATCCCAGCTGGTGAAGAGC |
| | | GAGCTGGAGGAGAAGAAGTCCGAGCTGCGGCACAAGCTGAAGTACGT |
| | | GCCCCACGAGTACATCGAGCTGATCGAGATCGCCAGGAACAGCACCC |
| | | AGGACCGCATCCTGGAGATGAAGGTGATGGAGTTCTTCATGAAGGTG |
| | | TACGGCTACAGGGGAAAGCACCTGGGCGGAAGCAGAAAGCCTGACGG |
| | | CGCCATCTATACAGTGGGCAGCCCCATCGATTACGGCGTGATCGTGG |
| | | ACACAAAGGCCTACAGCGGCGGCTACAATCTGCCTATCGGCCAGGCC |
| | | GACGAGATGGAGAGATACGTGGAGGAGAACCAGACCCGGGATAAGCA |
| | | CCTCAACCCCAACGAGTGGTGGAAGGTGTACCCTAGCAGCGTGACCG |
| | | AGTTCAAGTTCCTGTTCGTGAGCGGCCACTTCAAGGGCAACTACAAG |
| | | GCCCAGCTGACCAGGCTGAACCACATCACCAACTGCGACGGCGCCGT |
| | | GCTGAGCGTGGAGGAGCTGCTGATCGGCGGCGAGATGATCAAAGCCG |
| | | GCACCCTGACACTGGAGGAGGTGCGGCGCAAGTTCAACAACGGCGAG |
| | | ATCAACTTCAGATCT |
| 18 | Left ZFN | GATTACAAAGATCACGACGGAGATTACAAAGATCACGACATTGACTA |
| | with N-terminal | TAAGGACGACGACGATAAAATGGCTCCAAAGAAGAAAAGAAAAGTGG |
| | modifications | GGATCCATGGTGTACCCGCAGCAATGGCCGAACGACCCTTCCAATGC |
| | (comprising | AGAATATGTATGCAGAATTTTTCTCAGAGCGGGAACCTGGCGAGGCA |
| | 3xFLAG, NLS, | CATAAGAACCCATACAGGAGAGAAGCCATTCGCATGCGATATTTGCG |
| | ZFP-L, and | GTAGAAAATTTGCACTCAAACAAAATCTCTGTATGCACACTAAAATC |
| | FokI)(na) | CATACAGGTGAAAAGCCTTTTCAGTGCAGGATTTGTATGCAAAAATT |
| | Codon | TGCTTGGCAAAGTAACTTGCAGAACCACACAAAGATACACACAGGAG |
| | diversified | AGAAACCCTTCCAATGCCGAATCTGTATGCGCAACTTCAGTACATCC |
| | Version 1 | GGAAATTTGACTAGACATATTAGGACCCACACCGGCGAGAAGCCATT |
| | | TGCCTGCGATATTTGTGGACGGAAATTCGCACGACGCAGCCATCTGA |
| | | CCAGTCATACTAAGATTCATCTCCGCGGCAGCCAGCTTGTGAAGTCC |
| | | GAACTGGAGGAAAAGAAGAGCGAACTGCGCCACAAATTGAAATACGT |
| | | TCCGCATGAGTACATAGAGCTCATTGAAATCGCTAGAAACTCTACCC |
| | | AAGACAGGATACTGGAAATGAAAGTGATGGAATTTTTCATGAAAGTT |
| | | TATGGTTATAGGGGCAAACATCTGGGTGGCTCTCGCAAGCCCGATGG |
| | | GGCCATTTATACTGTCGGCTCACCTATCGACTATGGCGTCATTGTGG |
| | | ATACCAAGGCTTATTCTGGAGGATACAACCTGCCCATCGGACAAGCA |
| | | GACGAAATGGAAAGATACGTCGAGGAGAATCAAACCCGAGACAAGCA |
| | | TCTGAACCCAAACGAGTGGTGGAAAGTGTACCCGAGCAGCGTTACTG |
| | | AGTTCAAATTTCTCTTTGTAAGCGGACATTTTAAAGGGAATTACAAA |
| | | GCACAACTGACTAGGCTGAACCATATAACCAACTGTGACGGGGCCGT |
| | | ATTGAGTGTGGAAGAGCTTCTGATTGGAGGAGAGATGATTAAGGCTG |
| | | GCACACTGACTCTCGAAGAAGTGAGGCGCAAATTCAATAACGGTGAA |
| | | ATCAACTTCCGGTCT |
| 19 | Left ZFN | GACTACAAGGACCACGACGGTGACTACAAAGACCACGATATAGACTA |
| | with N-terminal | TAAAGATGACGATGATAAGATGGCACCTAAAAAAAAGCGGAAAGTGG |
| | modifications | GAATTCACGGCGTGCCCGCCGCCATGGCAGAGAGACCCTTTCAATGT |
| | (comprising | AGAATCTGTATGCAAAATTTCTCTCAGAGTGGTAACCTTGCAAGACA |
| | 3xFLAG, NLS, | CATCAGAACTCATACAGGTGAGAAGCCGTTTGCATGTGACATTTGCG |
| | ZFP-L, and | GTAGGAAATTTGCCTTGAAACAGAATCTTTGTATGCACACAAAAATC |
| | FokI)(na) | CATACTGGTGAAAAGCCATTCCAATGCCGCATCTGTATGCAAAAATT |
| | Codon | CGCGTGGCAGTCCAATTTGCAGAACCATACCAAGATTCACACGGGAG |
| | diversified | AAAAACCATTTCAGTGCCGCATCTGCATGCGCAACTTTTCTACATCA |
| | Version 2 | GGAAACCTTAGACGACATATTCGGACGCACACTGGAGAAAAACCATT |
| | | TGCTTGTGACATATGCGGCCGAAAATTTGCCAGACGCTCTCATCTCA |
| | | CCTCACATACTAAGATTCATTTGCGCGGAAGTCAGCTGGTGAAGAGT |
| | | GAATTGGAAGAAAAAAAGTCAGAGCTGAGACACAAACTGAAATATGT |
| | | TCCACACGAGTAGATCGAGCTTATCGAGATAGCAAGAAACTCCACCC |
| | | AGGACAGAATTTTGGAAATGAAAGTTATGGAATTCTTTATGAAAGTG |
| | | TATGGCTACAGGGGTAAACATCTGGGGGGATCAAGAAAGCCTGATGG |
| | | TGCAATTTACACAGTGGGCTCTCCTATCGACTACGGTGTGATCGTGG |
| | | ATACAAAGGCCTACTCTGGAGGATATAATTTGCCTATTGGACAAGCC |
| | | GATGAAATGGAAAGATATGTGGAGGAAAACCAGACTCGCGATAAGCA |
| | | CCTGAACCCAAATGAATGGTGGAAAGTGTACCCTTCATCTGTTACCG |
| | | AATTTAAATTTTTGTTCGTTTCCGGGCATTTCAAGGGGAACTACAAG |
| | | GCACAGCTGACGAGACTGAATCACATCACGAACTGCGACGGCGCTGT |
| | | ACTGTCCGTGGAAGAGCTTTTGATCGGGGGCGAAATGATTAAGGCCG |
| | | GCACACTGACGCTGGAGGAGGTGCGGCGAAAATTTAATAATGGCGAG |
| | | ATCAATTTTAGGAGT |
| 20 | Left ZFN | GATTACAAAGACCACGACGGAGACTACAAGGACCATGATATTGACTA |
| | with N- | CAAAGACGATGATGATAAGATGGCACCCAAAAAGAAGAGAAAAGTGG |
| | terminal | GAATCCACGGTGTACCGGCCGCGATGGCAGAGAGACCATTTCAGTGT |
| | modifications | AGAATCTGTATGCAGAACTTTTCCCAATCAGGAAACCTGGCACGACA |
| | (comprising | CATTAGAACCCATACTGGAGAAAAGCCGTTCGCTTGCGACATTTGCG |
| | 3xFLAG, NLS, | GTAGAAAATTTGCTTTGAAACAGAACTTGTGTATGCATACCAAGATT |
| | ZFP-L, and | CATACCGGCGAAAAACCATTTCAATGCAGGATTTGTATGCAGAAGTT |
| | FokI)(na) | CGCCTGGCAATCCAATTTGCAGAATCATACTAAAATTCATACCGGAG |
| | Codon | AAAAACCATTCCAATGCCGCATTTGTATGAGAAACTTTTCTACCTCT |
| | diversified | GGCAATCTCACCAGACATATCAGAACACACACAGGCGAGAAACCGTT |
| | Version 3 | CGCATGCGATATCTGTGGGCGAAAGTTTGCCAGAAGATCCCATCTCA |
| | | CATCACATACTAAAATACATTTGCGAGGAAGTCAACTGGTCAAGTCC |
| | | GAACTGGAGGAAAAAAAAAGTGAGCTGCGACACAAGTTGAAGTACGT |
| | | ACCACACGAATACATCGAGCTGATTGAGATAGCACGGAACTCTACCC |
| | | AGGATAGAATACTGGAGATGAAAGTTATGGAATTCTTTATGAAGGTG |
| | | TACGGATACAGGGGGAAGCATCTTGGCGGGAGCCGGAAACCAGACGG |
| | | AGCAATCTATACCGTCGGGTCACCTATAGACTATGGAGTTATTGTCG |
| | | ATACAAAGGCCTATTCAGGAGGTTATAATCTGCCAATCGGCCAAGCC |
| | | GACGAGATGGAGAGGTACGTGGAGGAAAATCAGACCAGAGACAAGCA |
| | | CCTGAACCCTAATGAATGGTGGAAAGTGTACCCTAGCAGCGTCACTG |
| | | AGTTCAAATTCCTGTTCGTCAGCGGTCATTTTAAAGGAAATTATAAA |
| | | GCCCAGCTCACTAGACTCAACCATATTACAAACTGCGACGGAGCCGT |
| | | ACTTAGCGTTGAAGAGTTGCTTATCGGAGGAGAGATGATCAAAGCCG |
| | | GAACCCTCACACTTGAAGAAGTGCGAAGAAAATTCAATAACGGAGAG |
| | | ATAAATTTTAGGAGT |
| 21 | Left ZFN | GACTATAAAGACCACGATGGCGACTAGAAAGACCACGACATCGATTA |
| | with N-terminal | CAAGGACGATGATGACAAAATGGCACCTAAGAAGAAGAGAAAAGTTG |
| | modifications | GAATACATGGAGTCCCCGCAGCAATGGCCGAGAGACCTTTTCAGTGC |
| | (comprising | AGGATTTGTATGCAAAACTTCTCTCAGTCCGGTAACCTGGCCCGGCA |
| | 3xFLAG, NLS, | CATACGAACACATACCGGCGAAAAACCCTTTGCTTGCGACATCTGCG |
| | ZFP-L, and | GAAGAAAGTTCGCTCTTAAACAGAACCTGTGCATGCATACAAAAATT |
| | FokI)(na) | CATACAGGTGAGAAGCCATTCCAATGCAGAATATGTATGCAGAAATT |
| | Codon | CGCCTGGCAAAGCAACCTGCAAAACCACACTAAGATCCACACAGGGG |
| | diversified | AAAAGCCTTTTCAATGTAGAATCTGTATGAGAAACTTTAGTACATCC |
| | Version 4 | GGAAATCTCACACGACATATCAGAACCCACACTGGAGAAAAACCTTT |
| | | TGCCTGCGACATCTGCGGAAGAAAATTCGCCCGAAGGTCCCACTTGA |
| | | CTAGTCATACCAAAATCCACTTGCGAGGCTCACAGCTGGTTAAATCC |
| | | GAACTTGAAGAAAAAAAAAGTGAACTGCGGCATAAACTGAAGTATGT |
| | | CCCCCATGAATATATCGAACTGATAGAAATCGCCCGAAATAGCACCC |
| | | AAGATAGAATCCTCGAAATGAAGGTTATGGAATTTTTCATGAAGGTC |
| | | TATGGATATAGGGGCAAGCACCTTGGCGGATCCCGGAAACCTGATGG |
| | | AGCTATCTAGACAGTGGGCTCACCAATAGACTATGGAGTTATCGTCG |
| | | ATACAAAAGCATACAGCGGAGGATACAATTTGCCAATAGGTCAAGCA |
| | | GATGAGATGGAAAGATACGTGGAGGAAAACCAAACAAGAGATAAGCA |
| | | TCTGAACCCCAACGAATGGTGGAAAGTGTACCCCAGTTCTGTAACCG |
| | | AATTTAAGTTCTTGTTCGTTTCAGGTCACTTCAAGGGTAATTACAAG |
| | | GCTCAACTGACTAGACTCAACCATATTACAAATTGCGATGGTGCTGT |
| | | GCTTTCCGTGGAAGAATTGCTGATTGGTGGAGAGATGATAAAAGCTG |
| | | GTACCCTCACCTTGGAAGAAGTGCGCAGAAAATTCAATAATGGCGAG |
| | | ATCAACTTCCGAAGT |
| 22 | Left ZFN | GATTATAAGGACCATGACGGAGACTATAAAGACCATGATATTGACTA |
| | with N- | CAAAGACGACGATGATAAGATGGCCCCCAAGAAGAAACGAAAAGTAG |
| | terminal | GAATCCATGGCGTGCCTGCAGCAATGGCAGAGAGACCATTTCAGTGC |
| | modifications | AGAATATGTATGCAAAACTTCTCCCAGAGCGGTAATCTGGCTAGGCA |
| | (comprising | TATTAGAACACACACCGGGGAAAAACCTTTCGCTTGCGATATATGTG |
| | 3xFLAG, NLS, | GTAGAAAGTTCGCCCTCAAACAGAATCTGTGCATGCACACTAAAATC |
| | ZFP-L, and | CATACAGGAGAAAAGCCCTTTCAGTGTAGAATTTGTATGCAGAAATT |
| | FokI)(na) | TGCTTGGCAGTCAAATTTGCAAAATCACACCAAAATACACACAGGAG |
| | Codon | AAAAACCATTTCAGTGTAGAATATGTATGAGAAATTTTTCCACTTCC |
| | diversified | GGAAATCTGACCAGACATATACGGACACACACTGGGGAAAAGCCCTT |
| | Version 5 | CGCTTGCGACATCTGCGGAAGAAAGTTCGCTAGACGGTCCCACTTGA |
| | | CATCCCACACTAAGATACATCTTCGCGGTAGCCAACTGGTGAAAAGT |
| | | GAACTGGAGGAAAAAAAATCTGAGCTGAGACATAAACTGAAATACGT |
| | | ACCACATGAATACATAGAACTTATAGAAATAGCTAGGAACTCCACCC |
| | | AGGACAGAATACTTGAAATGAAGGTCATGGAGTTTTTTATGAAAGTT |
| | | TACGGATACAGGGGCAAACACCTTGGAGGGTCTCGGAAGCCTGATGG |
| | | CGCAATTTATACCGTGGGTAGCCCTATAGATTATGGAGTGATTGTGG |
| | | ATACAAAGGCTTACAGTGGCGGCTATAATTTGCCTATCGGACAGGCC |
| | | GATGAGATGGAAAGATACGTTGAAGAAAACCAAACACGAGATAAGCA |
| | | TCTGAACCCCAATGAATGGTGGAAAGTGTATCCTTCAAGCGTTACCG |
| | | AGTTTAAGTTCCTCTTCGTTTCTGGGCATTTCAAGGGCAACTACAAA |
| | | GCTCAGCTTACAAGACTCAACCACATAACCAATTGTGATGGAGCAGT |
| | | CCTCAGCGTGGAAGAACTCCTTATTGGGGGTGAGATGATTAAAGCAG |
| | | GGACCCTTACTCTTGAAGAGGTTAGAAGAAAATTCAATAACGGAGAG |
| | | ATTAATTTTAGAAGT |
| 23 | Left ZFN | GACTATAAGGACCATGATGGAGACTATAAAGATCACGATATTGACTA |
| | with N-terminal | TAAAGATGATGATGATAAGATGGCACCTAAGAAGAAAAGAAAGGTCG |
| | modifications | GCATTCATGGTGTGCCTGCAGCCATGGCCGAACGCCCATTTCAATGT |
| | (comprising | AGAATTTGTATGCAGAATTTTTCACAATCAGGAAACCTGGCTAGACA |
| | 3xFLAG, NLS, | TATCAGAACACATACTGGAGAAAAGCCCTTTGCTTGTGATATCTGTG |
| | ZFP-L, and | GAAGGAAATTCGCCCTGAAACAAAACCTCTGTATGCACACAAAGATC |
| | FokI)(na) | CACACCGGCGAAAAGCCTTTCCAGTGTAGGATATGCATGCAAAAATT |
| | Codon | CGCCTGGCAGTCCAATCTGCAGAACCATACCAAAATTCATACTGGTG |
| | diversified | AAAAGCCATTTCAGTGCAGAATATGTATGAGAAACTTTAGCACTTCA |
| | Version 6 | GGAAATCTCACAAGACATATAAGAACACATACAGGGGAAAAACCTTT |
| | | TGCTTGCGATATCTGCGGCAGGAAATTCGCTCGGAGAAGTCATCTCA |
| | | CAAGCCATACAAAAATCCACCTGCGAGGAAGCCAGCTGGTCAAGTCT |
| | | GAACTGGAAGAAAAAAAAAGCGAACTGCGGCATAAACTCAAATACGT |
| | | CCCACATGAATACATTGAGCTCATCGAAATTGCTAGAAACTCTAGTC |
| | | AAGATAGGATATTGGAGATGAAGGTAATGGAATTCTTCATGAAGGTT |
| | | TATGGATATAGAGGAAAACATCTTGGAGGCAGTAGGAAACCCGATGG |
| | | CGCTATCTACACCGTAGGGAGTCCAATCGACTACGGCGTGATTGTTG |
| | | ACACCAAAGCCTATTCTGGAGGGTATAATCTCCCAATTGGACAGGCA |
| | | GATGAGATGGAAAGATATGTAGAAGAAAATCAGACAAGAGATAAGCA |
| | | CCTTAACCCTAACGAGTGGTGGAAAGTGTACCCAAGCAGTGTTACTG |
| | | AATTTAAATTTCTTTTTGTATCAGGACACTTTAAAGGCAATTACAAA |
| | | GCACAACTGACCAGACTCAATCACATTACCAATTGCGACGGAGCCGT |
| | | ACTGAGCGTGGAGGAGTTGCTGATCGGAGGCGAAATGATTAAAGCTG |
| | | GCACTCTGACCCTGGAAGAAGTAAGAAGAAAGTTCAATAATGGAGAA |
| | | ATAAACTTTCGCTCC |
| 24 | 2A peptide | GGCAGCGGAGAGGGCAGAGGAAGCCTGCTCACCTGCGGTGACGTGGA |
| | (T2A)(na) | GGAAAACCCTGGCCCT |
| 25 | Right ZFN | GACTACAAAGACCATGACGGTGATTATAAAGATCATGACATCGATTA |
| | with N-terminal | CAAGGATGACGATGACAAGATGGCCCCCAAGAAGAAGAGGAAGGTCG |
| | modifications | GCATTCATGGGGTACCCGCCGCTATGGCTGAGAGGCCCTTCCAGTGT |
| | (comprising | CGAATCTGCATGCGTAACTTCAGTCAGTCCTCCGACCTGTCCCGCCA |
| | 3xFLAG, NLS, | CATCCGCACCCACACCGGCGAGAAGCCTTTTGCCTGTGACATTTGTG |
| | ZFP-R, and | GGAGGAAATTTGCCCTGAAGCACAACCTGCTGACCCATACCAAGATA |
| | FokI)(na) | CACACGGGCGAGAAGCCCTTCCAGTGTCGAATCTGCATGCAGAACTT |
| | Not diversified | CAGTGACCAGTCCAACCTGCGCGCCCACATCCGCACCCACACCGGCG |
| | | AGAAGCCTTTTGCCTGTGACATTTGTGGGAGGAAATTTGCCCGCAAC |
| | | TTCTCCCTGACCATGCATACCAAGATACACACCGGAGAGCGCGGCTT |
| | | CCAGTGTCGAATCTGCATGCGTAACTTCAGTCTGCGCCACGACCTGG |
| | | AGCGCCACATCCGCACCCACACCGGCGAGAAGCCTTTTGCCTGTGAC |
| | | ATTTGTGGGAGGAAATTTGCCCACCGCTCCAACCTGAACAAGCATAC |
| | | CAAGATACACCTGCGGGGATCCCAGCTGGTGAAGAGCGAGCTGGAGG |
| | | AGAAGAAGTCCGAGCTGCGGCACAAGCTGAAGTACGTGCCCCACGAG |
| | | TACATCGAGCTGATCGAGATCGCCAGGAACAGCACCCAGGACCGCAT |
| | | CCTGGAGATGAAGGTGATGGAGTTCTTCATGAAGGTGTACGGCTACA |
| | | GGGGAAAGCACCTGGGCGGAAGCAGAAAGCCTGACGGCGCCATCTAT |
| | | ACAGTGGGCAGCCCCATCGATTACGGCGTGATCGTGGACACAAAGGC |
| | | CTACAGCGGCGGCTACAATCTGAGCATCGGCCAGGCCGACGAGATGC |
| | | AGAGATACGTGAAGGAGAACCAGACCCGGAATAAGCACATCAACCCC |
| | | AACGAGTGGTGGAAGGTGTACCCTAGCAGCGTGACCGAGTTCAAGTT |
| | | CCTGTTCGTGAGCGGCCACTTCAAGGGCAACTACAAGGCCCAGCTGA |
| | | CCAGGCTGAACCGCAAAACCAACTGCAATGGCGCCGTGCTGAGCGTG |
| | | GAGGAGCTGCTGATCGGCGGCGAGATGATCAAAGCCGGCACCCTGAC |
| | | ACTGGAGGAGGTGCGGCGCAAGTTCAACAACGGCGAGATCAACTTC |
| 26 | Right ZFN | GATTATAAAGATCATGACGGGGACTATAAGGATCACGACATAGACTA |
| | with N-terminal | CAAAGACGATGATGACAAAATGGCGCCTAAAAAGAAACGAAAAGTGG |
| | modifications | GCATTCACGGCGTACCTGCTGCTATGGCTGAAAGACCTTTTCAATGT |
| | (comprising | CGAATCTGCATGAGGAATTTTAGTCAGTCATCCGACCTGAGCAGACA |
| | 3xFLAG, NLS, | CATTCGAACCCATACTGGTGAAAAGCCATTTGCTTGCGATATATGTG |
| | ZFP-R, and | GGAGAAAATTTGCGTTGAAACACAATCTGCTGACCCATACCAAGATT |
| | FokI)(na) | CATACCGGAGAAAAACCATTCCAATGCCGCATTTGTATGCAGAACTT |
| | Codon | TAGTGACCAGTCAAATCTCCGCGCTCACATTCGAACCCACACTGGCG |
| | diversified | AAAAACCCTTTGCTTGTGACATTTGCGGTCGGAAGTTTGCCCGAAAT |
| | Version 1 | TTTTCTCTGACAATGCACACAAAAATCCACACCGGGGAACGCGGCTT |
| | | TCAATGTAGGATCTGTATGAGAAATTTTAGCCTTAGACATGATTTGG |
| | | AACGACATATCAGGACCCATACAGGCGAGAAACCATTTGCGTGCGAT |
| | | ATTTGTGGCAGGAAATTCGCACATAGAAGTAATCTGAACAAGCATAC |
| | | AAAAATTCATCTCAGAGGAAGTCAGCTGGTCAAAAGTGAACTGGAGG |
| | | AAAAAAAGAGCGAACTGAGACACAAACTGAAGTACGTGCCACACGAA |
| | | TATATTGAGCTGATTGAGATCGCGAGGAACTCAACACAGGACCGCAT |
| | | TCTGGAGATGAAAGTGATGGAGTTTTTCATGAAAGTATATGGATATA |
| | | GAGGAAAACACCTTGGGGGTAGCCGAAAGCCGGACGGGGCGATCTAC |
| | | ACTGTGGGGTCACCAATTGATTATGGCGTAATTGTCGATACCAAAGC |
| | | CTACAGTGGGGGGTACAATCTGAGTATAGGACAGGCTGATGAAATGC |
| | | AACGATACGTTAAGGAGAATCAGACTAGGAATAAACATATCAATCCA |
| | | AATGAATGGTGGAAAGTCTATCCCAGCAGCGTGACAGAATTTAAATT |
| | | TTTGTTTGTCAGTGGACACTTCAAGGGAAATTATAAGGCCCAGCTGA |
| | | CTAGACTGAATAGGAAAACCAATTGTAATGGCGCAGTGCTTTCAGTG |
| | | GAGGAACTGCTCATTGGAGGTGAGATGATCAAGGCTGGAACCCTGAC |
| | | GCTGGAGGAGGTGCGGAGGAAGTTTAACAATGGAGAAATTAACTTT |
| 27 | Right ZFN | GATTATAAAGACCATGATGGTGATTACAAGGACCATGACATCGATTA |
| | with N-terminal | TAAAGACGACGACGACAAAATGGCCCCTAAGAAAAAGAGAAAAGTCG |
| | modifications | GAATCCACGGTGTCCCAGCTGCCATGGCCGAGAGACCATTTCAATGT |
| | (comprising | CGGATTTGCATGCGCAATTTTTCCCAGTCCTCTGACCTTAGCCGGCA |
| | 3xFLAG, NLS, | TATTCGGACACACACAGGTGAAAAACCCTTCGCATGCGACATTTGCG |
| | ZFP-R, and | GAAGAAAATTCGCTCTGAAACACAACCTGCTTACCCATACAAAGATC |
| | FokI)(na) | CACACCGGCGAGAAACCGTTTCAATGCCGAATCTGTATGCAAAATTT |
| | Codon | TAGTGATCAAAGTAATCTGAGAGCACATATTAGGACTCACACGGGCG |
| | diversified | AGAAGCCATTTGCGTGTGATATCTGCGGCCGAAAATTCGCCCGGAAT |
| | Version 2 | TTCTCTCTGACAATGCACACCAAAATCCACACTGGGGAACGAGGCTT |
| | | TCAATGTAGAATATGTATGCGGAATTTCAGTCTGAGGCACGACCTGG |
| | | AGCGGCACATCAGAACTCACACCGGAGAAAAACCATTCGCTTGTGAT |
| | | ATTTGCGGGAGGAAGTTCGCCCATAGGAGCAATCTCAATAAACACAC |
| | | CAAAATACATCTTCGGGGTTCTCAACTGGTGAAATCCGAACTGGAAG |
| | | AAAAGAAATCAGAATTGCGGCATAAACTGAAGTATGTGCCCCATGAG |
| | | TACATAGAACTGATCGAGATCGCAAGGAACTCTACCCAGGACAGAAT |
| | | ACTTGAAATGAAGGTCATGGAATTTTTTATGAAAGTGTACGGCTACA |
| | | GAGGAAAACATTTGGGAGGCAGTCGAAAACCAGATGGCGCAATCTAT |
| | | ACAGTCGGGTCCCCCATAGATTACGGAGTGATTGTCGACACAAAAGC |
| | | CTATTCCGGAGGATATAACCTTAGTATCGGCCAGGCCGACGAGATGC |
| | | AACGCTATGTGAAAGAAAACCAAACAAGAAATAAACATATCAATCCA |
| | | AACGAGTGGTGGAAGGTATATCCAAGCAGTGTCACAGAATTCAAATT |
| | | CCTCTTCGTGAGTGGGCACTTTAAAGGCAACTACAAAGCTCAATTGA |
| | | CCAGGCTCAATCGGAAAACTAATTGCAATGGCGCAGTCCTTAGCGTC |
| | | GAAGAATTGCTGATTGGCGGGGAAATGATTAAAGCAGGAACTTTGAC |
| | | CTTGGAGGAAGTACGGAGAAAGTTTAACAACGGCGAGATTAATTTT |
| 28 | Right ZFN | GATTATAAGGATCATGATGGAGACTATAAGGATCATGACATAGATTA |
| | with N-terminal | CAAAGATGACGATGACAAGATGGCACCCAAGAAGAAAAGAAAAGTAG |
| | modifications | GAATTCACGGAGTCCCTGCCGCCATGGCCGAGCGCCCCTTCCAATGC |
| | (comprising | CGCATATGCATGAGAAATTTGAGCCAAAGTAGCGACCTGTCACGACA |
| | 3xFLAG, NLS, | CATTAGAACTCATACGGGGGAGAAGCCATTTGCTTGCGATATTTGTG |
| | ZFP-R, and | GCAGAAAATTCGCACTCAAACACAACCTGCTCACACACACCAAGATA |
| | FokI)(na) | CACACGGGAGAGAAGCCCTTCCAATGTAGAATATGTATGCAAAATTT |
| | Codon | CAGCGACCAAAGTAATTTGAGAGCGCATATTCGAACTCACACCGGCG |
| | diversified | AAAAACCATTTGCCTGCGATATTTGTGGGAGGAAATTTGCCAGGAAT |
| | Version 3 | TTTTCACTCACCATGCACACTAAGATCCACACTGGCGAGCGCGGCTT |
| | | CCAATGCAGAATCTGTATGCGAAACTTCAGTCTGCGGCATGACCTGG |
| | | AAAGACATATAAGAACCCACACCGGAGAAAAACCCTTTGCCTGCGAC |
| | | ATATGTGGTAGAAAATTCGCACATCGGAGTAACCTTAACAAACATAC |
| | | AAAGATCCACTTGAGAGGCAGTCAGCTGGTGAAATCTGAGCTGGAAG |
| | | AGAAGAAATCTGAACTGCGACATAAATTGAAGTACGTCCCACACGAG |
| | | TACATCGAGTTGATCGAAATTGCCCGGAATAGCACCCAGGATAGAAT |
| | | ATTGGAAATGAAAGTAATGGAGTTTTTTATGAAGGTTTATGGTTACA |
| | | GAGGCAAGCACCTTGGAGGAAGCAGGAAACCAGATGGGGCGATTTAC |
| | | ACCGTTGGGAGTCCCATCGATTACGGAGTCATCGTGGACACAAAGGC |
| | | CTATTCCGGAGGCTACAACCTCAGTATCGGGCAAGCCGATGAGATGC |
| | | AGAGATATGTTAAAGAAAATCAGACGCGAAACAAGCACATTAACCCA |
| | | AACGAATGGTGGAAAGTTTACCCTAGCTCAGTGACAGAATTTAAGTT |
| | | TCTGTTTGTCAGCGGCCACTTCAAGGGGAATTATAAAGCACAACTGA |
| | | CCCGCCTGAACCGAAAAACCAACTGTAACGGTGCTGTGCTGAGTGTC |
| | | GAAGAGTTGCTTATCGGAGGAGAGATGATAAAGGCCGGCACACTGAC |
| | | GCTTGAAGAGGTACGGCGAAAATTCAATAACGGAGAGATTAATTTT |
| 29 | Right ZFN | GACTACAAAGATCATGATGGCGACTACAAAGATCATGATATAGATTA |
| | with N-terminal | CAAAGACGATGACGACAAAATGGCTCCAAAAAAAAAACGCAAGGTTG |
| | modifications | GAATACACGGTGTACCTGCCGCTATGGCTGAAAGACCTTTCCAGTGT |
| | (comprising | AGGATTTGCATGAGAAATTTTTCCCAATCATCCGACCTTTCAAGGCA |
| | 3xFLAG, NLS, | TATTAGGACACACACCGGGGAAAAGCCATTTGCTTGTGATATCTGCG |
| | ZFP-R, and | GGCGCAAATTTGCTCTTAAGCACAATCTTCTTACCCACACCAAAATT |
| | FokI)(na) | CATACAGGAGAAAAACCTTTTCAATGTAGAATCTGCATGCAAAACTT |
| | Codon | TTCCGATCAGTCAAATCTTAGAGCTCATATCAGAACCCATACCGGGG |
| | diversified | AGAAACCCTTTGCCTGCGACATATGCGGAAGAAAATTTGCTAGGAAC |
| | Version 4 | TTTAGTCTGACCATGCATACCAAAATTCATACCGGCGAACGCGGTTT |
| | | CCAGTGCAGGATTTGTATGAGAAATTTCTCACTGCGGCATGATCTTG |
| | | AAAGACACATACGAACTCATACCGGAGAAAAGCCATTCGCTTGCGAT |
| | | ATTTGTGGTAGAAAATTTGCCCACAGGTCTAACCTTAATAAGCACAC |
| | | CAAGATTCATCTCAGAGGATCTCAGCTGGTCAAATCAGAACTTGAAG |
| | | AGAAAAAAAGCGAACTGAGACATAAACTGAAGTACGTGCCTCATGAA |
| | | TACATAGAGCTCATTGAAATAGCTAGGAATAGTAGACAGGACAGGAT |
| | | ACTTGAAATGAAGGTAATGGAATTTTTCATGAAGGTTTATGGATACC |
| | | GGGGGAAACATCTCGGGGGCAGCAGAAAACCAGACGGAGCAATTTAT |
| | | ACTGTCGGGAGTCCTATAGATTATGGCGTTATCGTCGATACAAAGGC |
| | | CTATTCCGGTGGGTACAACCTCTCAATTGGTCAGGCTGATGAGATGC |
| | | AAAGATACGTCAAAGAAAACCAAACCAGAAATAAACATATAAATCCC |
| | | AATGAATGGTGGAAAGTATACCCAAGTTCCGTGACTGAATTCAAGTT |
| | | CCTTTTCGTGTCTGGCCACTTTAAAGGAAATTATAAAGCACAATTGA |
| | | CTAGACTGAATAGAAAAACAAACTGTAACGGCGCAGTGCTGTCAGTG |
| | | GAAGAACTGCTCATAGGTGGAGAGATGATCAAGGCCGGGACACTTAC |
| | | TCTTGAGGAAGTTAGAAGGAAGTTCAACAACGGCGAAATCAACTTT |
| 30 | Right ZFN | GATTACAAAGACCATGATGGCGACTATAAAGACCATGACATCGACTA |
| | with N-terminal | CAAGGATGATGATGATAAAATGGCTCCAAAGAAAAAGAGGAAGGTGG |
| | modifications | GAATACATGGAGTACCAGCAGCTATGGCCGAACGCCCTTTTCAATGC |
| | (comprising | AGAATATGTATGCGAAACTTCTCCCAAAGCTCTGATCTGTCAAGGCA |
| | 3xFLAG, NLS, | CATACGGACACACACCGGCGAAAAACCCTTTGCATGTGACATTTGTG |
| | ZFP-R, and | GAAGAAAATTCGCACTTAAACACAATCTCCTGACTCATACAAAAATA |
| | FokI)(na) | CATACAGGCGAAAAACCTTTCCAGTGCAGAATCTGTATGCAGAACTT |
| | Codon | TTCCGACCAATCCAATCTTCGCGCCCACATTAGAACTCACACAGGGG |
| | diversified | AGAAACCTTTCGCTTGCGACATATGCGGAAGAAAATTTGCCAGAAAT |
| | Version 5 | TTTTCACTTACAATGCACACAAAAATACATACTGGGGAAAGAGGGTT |
| | | TCAATGTCGAATCTGTATGAGAAATTTCAGTCTGCGCCATGATCTGG |
| | | AGAGACATATAAGAACACACACAGGAGAGAAACCTTTTGCTTGTGAC |
| | | ATATGCGGCCGAAAGTTTGCTCATAGATCTAATCTTAACAAACATAC |
| | | AAAGATCCATCTTCGGGGTTCACAACTGGTCAAGTCAGAATTGGAAG |
| | | AGAAAAAATCTGAGCTGAGGCACAAATTGAAATACGTTCCTCACGAG |
| | | TATATTGAACTTATCGAGATAGCCCGCAATAGTAGACAAGATAGAAT |
| | | CTTGGAGATGAAAGTTATGGAATTCTTTATGAAAGTCTATGGCTATA |
| | | GGGGAAAACACCTGGGGGGTAGCAGGAAACCTGATGGAGCTATCTAT |
| | | ACCGTAGGATCACCTATTGATTATGGAGTAATTGTGGACACTAAGGC |
| | | ATATTCCGGAGGATATAATTTGAGTATTGGTCAGGCCGACGAAATGC |
| | | AACGATACGTGAAGGAAAATCAGACCCGCAACAAACACATTAATCCC |
| | | AATGAATGGTGGAAGGTATACCCTAGTAGCGTTACAGAGTTTAAATT |
| | | CCTTTTCGTCAGCGGCCACTTTAAAGGAAATTATAAAGCACAACTCA |
| | | CCAGACTTAATCGAAAAACTAACTGTAACGGCGCCGTACTGTCAGTG |
| | | GAGGAGCTGCTCATTGGAGGCGAGATGATCAAGGCCGGTACTCTCAC |
| | | ACTGGAAGAAGTTAGAAGAAAGTTCAACAACGGGGAAATTAATTTC |
| 31 | Right ZFN | GACTACAAGGACCACGACGGAGACTATAAAGACCATGATATAGATTA |
| | with N-terminal | CAAGGACGATGACGATAAAATGGCACCCAAAAAGAAAAGAAAGGTGG |
| | modifications | GTATTCACGGAGTTCCCGCTGCTATGGCTGAGAGACCTTTCCAATGT |
| | (comprising | AGGATCTGTATGCGAAACTTCTCCCAGAGCTCCGACCTGAGTCGCCA |
| | 3xFLAG, NLS, | TATAAGAACCCATACCGGAGAAAAACCATTTGCTTGTGACATTTGTG |
| | ZFP-R, and | GCAGAAAGTTCGCTCTTAAACACAACCTGCTTACACATACTAAAATA |
| | FokI)(na) | CACACAGGGGAGAAACCCTTTCAATGCCGGATCTGTATGCAAAACTT |
| | Codon | TAGCGATCAATCAAACTTGCGAGCCCATATCCGCACTCACACCGGCG |
| | diversified | AGAAGCCTTTTGCATGCGATATATGTGGACGGAAATTTGCTAGAAAC |
| | Version 6 | TTCTCATTGACCATGCATACAAAAATACACACCGGGGAACGAGGATT |
| | | TCAATGTCGAATTTGTATGAGAAATTTTAGCCTTAGGCACGACTTGG |
| | | AACGGCACATAAGAACCCACACCGGAGAGAAGCCTTTTGCTTGTGAT |
| | | ATTTGCGGCAGAAAGTTCGCCCATCGCAGCAATCTTAACAAGCACAC |
| | | CAAGATTCATTTGAGAGGTTCCCAGCTGGTCAAAAGCGAACTTGAAG |
| | | AAAAGAAATCCGAGCTTAGACACAAACTGAAATACGTGCCTCACGAG |
| | | TATATTGAGCTGATTGAAATAGCAAGGAATTCAACACAAGACAGGAT |
| | | CCTCGAAATGAAGGTTATGGAGTTTTTCATGAAAGTTTACGGCTACA |
| | | GAGGGAAGCATCTGGGCGGATCAAGAAAACCAGACGGCGCAATCTAC |
| | | ACAGTTGGATCCCCAATAGATTACGGAGTGATTGTTGACACCAAGGC |
| | | TTATTCAGGAGGTTACAATCTGTCCATTGGTCAGGCCGATGAAATGC |
| | | AAAGATATGTTAAGGAAAATCAAACTCGAAACAAACACATTAATCCA |
| | | AACGAATGGTGGAAAGTATATCCAAGCTCCGTCACTGAATTTAAATT |
| | | TTTGTTTGTATCCGGACATTTTAAGGGCAACTATAAGGCTCAACTGA |
| | | CCAGACTGAATAGGAAGACCAATTGTAACGGAGCTGTACTCAGCGTG |
| | | GAAGAACTGCTTATTGGAGGCGAAATGATTAAGGCTGGCACACTTAC |
| | | ACTCGAAGAAGTTAGAAGAAAATTCAACAATGGTGAGATAAACTTC |
| 32 | WPREmut6 | AATCAACCTCTGGATTACAAAATTTGTGAAAGATTGACTGATATTCT |
| | 3′UTR(na) | TAACTATGTTGCTCCTTTTACGCTGTGTGGATATGCTGCTTTAATGC |
| | | CTCTGTATCATGCTATTGCTTCCCGTACGGCTTTCGTTTTCTCCTCC |
| | | TTGTATAAATCCTGGTTGCTGTCTCTTTATGAGGAGTTGTGGCCCGT |
| | | TGTCCGTCAACGTGGCGTGGTGTGCTCTGTGTTTGCTGACGCAACCC |
| | | CCACTGGCTGGGGCATTGCCACCACCTGTCAACTCCTTTCTGGGACT |
| | | TTCGCTTTCCCCCTCCCGATCGCCACGGCAGAACTCATCGCCGCCTG |
| | | CCTTGCCCGCTGCTGGACAGGGGCTAGGTTGCTGGGCACTGATAATT |
| | | CCGTGGTGTTGTCGGGGAAATCATCGTCCTTTCCTTGGCTGCTCGCC |
| | | TGTGTTGCCAACTGGATCCTGCGCGGGACGTCCTTCTGCTACGTCCC |
| | | TTCGGCTCTCAATCCAGCGGACCTCCCTTCCCGAGGCCTTCTGCCGG |
| | | TTCTGCGGCCTCTCCCGCGTCTTCGCTTTCGGCCTCCGACGAGTCGG |
| | | ATCTCCCTTTGGGCCGCCTCCCCGCCTG |
| 33 | Polyadenylation | CTGTGCCTTCTAGTTGCCAGCCATCTGTTGTTTGCCCCTCCCCCGTG |
| | signal (na) | CCTTCCTTGACCCTGGAAGGTGCCACTCCCACTGTCCTTTCCTAATA |
| | | AAATGAGGAAATTGCATCGCATTGTCTGAGTAGGTGTCATTCTATTC |
| | | TGGGGGGTGGGGTGGGGCAGGACAGCAAGGGGGAGGATTGGGAAGAC |
| | | AATAGCAGGCATGCTGGGGATGCGGTGGGCTCTAT |
| 34 | 3′ ITR (na) | AGGAACCCCTAGTGATGGAGTTGGCCACTCCCTCTCTGCGCGCTCGC |
| | | TCGCTCACTGAGGCCGCCCGGGCTTTGCCCGGGCGGCCTCAGTGAGC |
| | | GAGCGAGCGCGCAG |
| 50 | 3xFLAG (na) | GATTATAAAGACCATGATGGTGATTACAAGGACCATGACATCGATTA |
| | | TAAAGACGACGACGACAAA |
| 51 | 3xFLAG (na) | GATTATAAGGATCATGATGGAGACTATAAGGATCATGACATAGATTA |
| | | CAAAGATGACGATGACAAG |
| 52 | 3xFLAG (na) | GACTACAAAGATCATGATGGCGACTACAAAGATCATGATATAGATTA |
| | | CAAAGACGATGACGACAAA |
| 53 | 3xFLAG (na) | GATTAGAAAGACCATGATGGCGACTATAAAGACCATGACATCGACTA |
| | | CAAGGATGATGATGATAAA |
| 54 | 3xFLAG (na) | GACTAGAAGGACCACGACGGAGACTATAAAGACCATGATATAGATTA |
| | | CAAGGACGATGACGATAAA |
| 55 | 3xFLAG (na) | GACTACAAGGACCACGACGGTGACTACAAAGACCACGATATAGACTA |
| | | TAAAGATGACGATGATAAG |
| 56 | 3xFLAG (na) | GACTATAAAGACCACGATGGCGACTACAAAGACCACGACATCGATTA |
| | | CAAGGACGATGATGACAAA |
| 57 | 3xFLAG (na) | GATTATAAGGACCATGACGGAGACTATAAAGACCATGATATTGACTA |
| | | CAAAGACGACGATGATAAG |
| 58 | 3xFLAG (na) | GACTATAAGGACCATGATGGAGACTATAAAGATCACGATATTGACTA |
| | | TAAAGATGATGATGATAAG |
| 59 | Nuclear | CCTAAAAAGAAACGAAAAGTGGGCATTCAC |
| | Localization | |
| | Sequence | |
| | (NLS)(na) | |
| 60 | Nuclear | CCCAAGAAGAAGAGGAAGGTCGGCATTCAT |
| | Localization | |
| | Sequence | |
| | (NLS)(na) | |
| 61 | Nuclear | CCTAAGAAAAAGAGAAAAGTCGGAATCCAC |
| | Localization | |
| | Sequence | |
| | (NLS)(na) | |
| 62 | Nuclear | CCCAAGAAGAAAAGAAAAGTAGGAATTCAC |
| | Localization | |
| | Sequence | |
| | (NLS)(na) | |
| 63 | Nuclear | CCAAAAAAAAAACGCAAGGTTGGAATACAC |
| | Localization | |
| | Sequence | |
| | (NLS)(na) | |
| 64 | Nuclear | CCAAAGAAAAAGAGGAAGGTGGGAATACAT |
| | Localization | |
| | Sequence | |
| | (NLS)(na) | |
| 65 | Nuclear | CCCAAAAAGAAAAGAAAGGTGGGTATTCAC |
| | Localization | |
| | Sequence | |
| | (NLS)(na) | |
| 66 | Nuclear | CCAAAGAAGAAAAGAAAAGTGGGGATCCAT |
| | Localization | |
| | Sequence | |
| | (NLS)(na) | |
| 67 | Nuclear | CCTAAAAAAAAGCGGAAAGTGGGAATTCAC |
| | Localization | |
| | Sequence | |
| | (NLS)(na) | |
| 68 | Nuclear | CCTAAGAAGAAGAGAAAAGTTGGAATACAT |
| | Localization | |
| | Sequence | |
| | (NLS)(na) | |
| 69 | Nuclear | CCCAAGAAGAAACGAAAAGTAGGAATCCAT |
| | Localization | |
| | Sequence | |
| | (NLS)(na) | |
| 70 | Nuclear | CCTAAGAAGAAAAGAAAGGTCGGCATTCAT |
| | Localization | |
| | Sequence | |
| | (NLS)(na) | |
| 155 | Nuclear | CCCAAAAAGAAGAGAAAAGTGGGAATCCAC |
| | Localization | |
| | Sequence | |
| | (NLS)(na) | |
| 71 | Left ZFN | GCCGCTATGGCTGAGAGGCCCTTCCAGTGTCGAATCTGCATGCAGAA |
| | (ZFN-L | CTTCAGTCAGTCCGGCAACCTGGCCCGCCACATCCGCACCCACACCG |
| | comprises ZFP- | GCGAGAAGCCTTTTGCCTGTGACATTTGTGGGAGGAAATTTGCCCTG |
| | L and FokI) | AAGCAGAACCTGTGTATGCATACCAAGATACACACGGGCGAGAAGCC |
| | (na) | CTTCCAGTGTCGAATCTGCATGCAGAAGTTTGCCTGGCAGTCCAACC |
| | Not diversified | TGCAGAACCATACCAAGATACACACGGGCGAGAAGCCCTTCCAGTGT |
| | | CGAATCTGCATGCGTAACTTCAGTACCTCCGGCAACCTGACCCGCCA |
| | | CATCCGCACCCACACCGGCGAGAAGCCTTTTGCCTGTGACATTTGTG |
| | | GGAGGAAATTTGCCCGCCGCTCCCACCTGACCTCCCATACCAAGATA |
| | | CACCTGCGGGGATCCCAGCTGGTGAAGAGCGAGCTGGAGGAGAAGAA |
| | | GTCCGAGCTGCGGCACAAGCTGAAGTACGTGCCCCACGAGTACATCG |
| | | AGCTGATCGAGATCGCCAGGAACAGCACCCAGGACCGCATCCTGGAG |
| | | ATGAAGGTGATGGAGTTCTTCATGAAGGTGTACGGCTACAGGGGAAA |
| | | GCACCTGGGCGGAAGCAGAAAGCCTGACGGCGCCATCTATACAGTGG |
| | | GCAGCCCCATCGATTACGGCGTGATCGTGGACACAAAGGCCTACAGC |
| | | GGCGGCTACAATCTGCCTATCGGCCAGGCCGACGAGATGGAGAGATA |
| | | CGTGGAGGAGAACCAGACCCGGGATAAGCACCTCAACCCCAACGAGT |
| | | GGTGGAAGGTGTACCCTAGCAGCGTGACCGAGTTCAAGTTCCTGTTC |
| | | GTGAGCGGCCACTTCAAGGGCAACTACAAGGCCCAGCTGACCAGGCT |
| | | GAACCACATCACCAACTGCGACGGCGCCGTGCTGAGCGTGGAGGAGC |
| | | TGCTGATCGGCGGCGAGATGATCAAAGCCGGCACCCTGACACTGGAG |
| | | GAGGTGCGGCGCAAGTTCAACAACGGCGAGATCAACTTCAGATCT |
| 72 | Left ZFN | GCAGCAATGGCCGAACGACCCTTCCAATGCAGAATATGTATGCAGAA |
| | (ZFN-L | TTTTTCTCAGAGCGGGAACCTGGCGAGGCACATAAGAACCCATACAG |
| | comprises ZFP- | GAGAGAAGCCATTCGCATGCGATATTTGCGGTAGAAAATTTGCACTC |
| | L and FokI) | AAACAAAATCTCTGTATGCACACTAAAATCCATACAGGTGAAAAGCC |
| | (na) | TTTTCAGTGCAGGATTTGTATGCAAAAATTTGCTTGGCAAAGTAACT |
| | Codon | TGCAGAACCACACAAAGATACACACAGGAGAGAAACCCTTCCAATGC |
| | diversified | CGAATCTGTATGCGCAACTTCAGTACATCCGGAAATTTGACTAGACA |
| | Version 1 | TATTAGGACCCACACCGGCGAGAAGCCATTTGCCTGCGATATTTGTG |
| | | GACGGAAATTCGCACGACGCAGCCATCTGACCAGTCATACTAAGATT |
| | | CATCTCCGCGGCAGCCAGCTTGTGAAGTCCGAACTGGAGGAAAAGAA |
| | | GAGCGAACTGCGCCACAAATTGAAATACGTTCCGCATGAGTAGATAG |
| | | AGCTCATTGAAATCGCTAGAAACTCTACCCAAGACAGGATACTGGAA |
| | | ATGAAAGTGATGGAATTTTTCATGAAAGTTTATGGTTATAGGGGCAA |
| | | ACATCTGGGTGGCTCTCGCAAGCCCGATGGGGCCATTTATACTGTCG |
| | | GCTCACCTATCGACTATGGCGTCATTGTGGATACCAAGGCTTATTCT |
| | | GGAGGATACAACCTGCCCATCGGACAAGCAGACGAAATGGAAAGATA |
| | | CGTCGAGGAGAATCAAACCCGAGACAAGCATCTGAACCCAAACGAGT |
| | | GGTGGAAAGTGTACCCGAGCAGCGTTACTGAGTTCAAATTTCTCTTT |
| | | GTAAGCGGACATTTTAAAGGGAATTACAAAGCACAACTGAGTAGGCT |
| | | GAACCATATAACCAACTGTGACGGGGCCGTATTGAGTGTGGAAGAGC |
| | | TTCTGATTGGAGGAGAGATGATTAAGGCTGGCACACTGACTCTCGAA |
| | | GAAGTGAGGCGCAAATTCAATAACGGTGAAATCAACTTCCGGTCT |
| 73 | Left ZFN | GCCGCCATGGCAGAGAGACCCTTTCAATGTAGAATCTGTATGCAAAA |
| | (ZFN-L | TTTCTCTCAGAGTGGTAACCTTGCAAGACACATCAGAACTCATACAG |
| | comprises ZFP- | GTGAGAAGCCGTTTGCATGTGACATTTGCGGTAGGAAATTTGCCTTG |
| | L and FokI) | AAACAGAATCTTTGTATGCACACAAAAATCCATACTGGTGAAAAGCC |
| | (na) | ATTCCAATGCCGCATCTGTATGCAAAAATTCGCGTGGCAGTCCAATT |
| | Codon | TGCAGAACCATACCAAGATTCACACGGGAGAAAAACCATTTCAGTGC |
| | diversified | CGCATCTGCATGCGCAACTTTTCTACATCAGGAAACCTTACACGACA |
| | Version 2 | TATTCGGACGCACACTGGAGAAAAACCATTTGCTTGTGACATATGCG |
| | | GCCGAAAATTTGCCAGACGCTCTCATCTCACCTCACATACTAAGATT |
| | | CATTTGCGCGGAAGTCAGCTGGTGAAGAGTGAATTGGAAGAAAAAAA |
| | | GTCAGAGCTGAGACACAAACTGAAATATGTTCCACACGAGTACATCG |
| | | AGCTTATCGAGATAGCAAGAAACTCCACCCAGGACAGAATTTTGGAA |
| | | ATGAAAGTTATGGAATTCTTTATGAAAGTGTATGGCTACAGGGGTAA |
| | | ACATCTGGGGGGATCAAGAAAGCCTGATGGTGCAATTTACACAGTGG |
| | | GCTCTCCTATCGACTACGGTGTGATCGTGGATACAAAGGCCTACTCT |
| | | GGAGGATATAATTTGCCTATTGGACAAGCCGATGAAATGGAAAGATA |
| | | TGTGGAGGAAAACCAGACTCGCGATAAGCACCTGAACCCAAATGAAT |
| | | GGTGGAAAGTGTACCCTTCATCTGTTACCGAATTTAAATTTTTGTTC |
| | | GTTTCCGGGCATTTCAAGGGGAACTACAAGGCACAGCTGACGAGACT |
| | | GAATCACATCACGAACTGCGACGGCGCTGTACTGTCCGTGGAAGAGC |
| | | TTTTGATCGGGGGCGAAATGATTAAGGCCGGCACACTGACGCTGGAG |
| | | GAGGTGCGGCGAAAATTTAATAATGGCGAGATCAATTTTAGGAGT |
| 74 | Left ZFN | GCCGCGATGGCAGAGAGACCATTTCAGTGTAGAATCTGTATGCAGAA |
| | (ZFN-L | CTTTTCCCAATCAGGAAACCTGGCACGACACATTAGAACCCATACTG |
| | comprises ZFP- | GAGAAAAGCCGTTCGCTTGCGACATTTGCGGTAGAAAATTTGCTTTG |
| | L and FokI) | AAACAGAACTTGTGTATGCATACCAAGATTCATACCGGCGAAAAACC |
| | (na) | ATTTCAATGCAGGATTTGTATGCAGAAGTTCGCCTGGCAATCCAATT |
| | Codon | TGCAGAATCATACTAAAATTCATACCGGAGAAAAACCATTCCAATGC |
| | diversified | CGCATTTGTATGAGAAACTTTTCTACCTCTGGCAATCTCACCAGACA |
| | Version 3 | TATCAGAACACACACAGGCGAGAAACCGTTCGCATGCGATATCTGTG |
| | | GGCGAAAGTTTGCCAGAAGATCCCATCTCACATCACATACTAAAATA |
| | | CATTTGCGAGGAAGTCAACTGGTCAAGTCCGAACTGGAGGAAAAAAA |
| | | AAGTGAGCTGCGACACAAGTTGAAGTACGTACCACACGAATACATCG |
| | | AGCTGATTGAGATAGCACGGAACTCTACCCAGGATAGAATACTGGAG |
| | | ATGAAAGTTATGGAATTCTTTATGAAGGTGTACGGATACAGGGGGAA |
| | | GCATCTTGGCGGGAGCCGGAAACCAGACGGAGCAATCTATACCGTCG |
| | | GGTCACCTATAGACTATGGAGTTATTGTCGATACAAAGGCCTATTCA |
| | | GGAGGTTATAATCTGCCAATCGGCCAAGCCGACGAGATGGAGAGGTA |
| | | CGTGGAGGAAAATCAGACCAGAGACAAGCACCTGAACCCTAATGAAT |
| | | GGTGGAAAGTGTACCCTAGCAGCGTCACTGAGTTCAAATTCCTGTTC |
| | | GTCAGCGGTCATTTTAAAGGAAATTATAAAGCCCAGCTCACTAGACT |
| | | CAACCATATTACAAACTGCGACGGAGCCGTACTTAGCGTTGAAGAGT |
| | | TGCTTATCGGAGGAGAGATGATCAAAGCCGGAACCCTCACACTTGAA |
| | | GAAGTGCGAAGAAAATTCAATAACGGAGAGATAAATTTTAGGAGT |
| 75 | Left ZFN | GCAGCAATGGCCGAGAGACCTTTTCAGTGCAGGATTTGTATGCAAAA |
| | (ZFN-L | CTTCTCTCAGTCCGGTAACCTGGCCCGGCACATACGAACACATACCG |
| | comprises ZFP- | GCGAAAAACCCTTTGCTTGCGACATCTGCGGAAGAAAGTTCGCTCTT |
| | L and FokI) | AAACAGAACCTGTGCATGCATACAAAAATTCATACAGGTGAGAAGCC |
| | (na) | ATTCCAATGCAGAATATGTATGCAGAAATTCGCCTGGCAAAGCAACC |
| | Codon | TGCAAAACCACACTAAGATCCACACAGGGGAAAAGCCTTTTCAATGT |
| | diversified | AGAATCTGTATGAGAAACTTTAGTACATCCGGAAATCTCACACGACA |
| | Version 4 | TATCAGAACCCACACTGGAGAAAAACCTTTTGCCTGCGACATCTGCG |
| | | GAAGAAAATTCGCCCGAAGGTCCCACTTGACTAGTCATACCAAAATC |
| | | CACTTGCGAGGCTCACAGCTGGTTAAATCCGAACTTGAAGAAAAAAA |
| | | AAGTGAACTGCGGCATAAACTGAAGTATGTCCCCCATGAATATATCG |
| | | AACTGATAGAAATCGCCCGAAATAGCACCCAAGATAGAATCCTCGAA |
| | | ATGAAGGTTATGGAATTTTTCATGAAGGTCTATGGATATAGGGGCAA |
| | | GCACCTTGGCGGATCCCGGAAACCTGATGGAGCTATCTACACAGTGG |
| | | GCTCACCAATAGACTATGGAGTTATCGTCGATACAAAAGCATACAGC |
| | | GGAGGATACAATTTGCCAATAGGTCAAGCAGATGAGATGGAAAGATA |
| | | CGTGGAGGAAAACCAAACAAGAGATAAGCATCTGAACCCCAACGAAT |
| | | GGTGGAAAGTGTACCCCAGTTCTGTAACCGAATTTAAGTTCTTGTTC |
| | | GTTTCAGGTCACTTCAAGGGTAATTACAAGGCTCAACTGACTAGACT |
| | | CAACCATATTACAAATTGCGATGGTGCTGTGCTTTCCGTGGAAGAAT |
| | | TGCTGATTGGTGGAGAGATGATAAAAGCTGGTACCCTCACCTTGGAA |
| | | GAAGTGCGCAGAAAATTCAATAATGGCGAGATCAACTTCCGAAGT |
| 76 | Left ZFN | GCAGCAATGGCAGAGAGACCATTTCAGTGCAGAATATGTATGCAAAA |
| | (ZFN-L | CTTCTCCCAGAGCGGTAATCTGGCTAGGCATATTAGAACACACACCG |
| | comprises ZFP- | GGGAAAAACCTTTCGCTTGCGATATATGTGGTAGAAAGTTCGCCCTC |
| | L and FokI) | AAACAGAATCTGTGCATGCACACTAAAATCCATACAGGAGAAAAGCC |
| | (na) | CTTTCAGTGTAGAATTTGTATGCAGAAATTTGCTTGGCAGTCAAATT |
| | Codon | TGCAAAATCACACCAAAATACACACAGGAGAAAAACCATTTCAGTGT |
| | diversified | AGAATATGTATGAGAAATTTTTCCACTTCCGGAAATCTGACCAGACA |
| | Version 5 | TATACGGACACACACTGGGGAAAAGCCCTTCGCTTGCGACATCTGCG |
| | | GAAGAAAGTTCGCTAGACGGTCCCACTTGACATCCCACACTAAGATA |
| | | CATCTTCGCGGTAGCCAACTGGTGAAAAGTGAACTGGAGGAAAAAAA |
| | | ATCTGAGCTGAGACATAAACTGAAATACGTACCACATGAATACATAG |
| | | AACTTATAGAAATAGCTAGGAACTCCACCCAGGACAGAATACTTGAA |
| | | ATGAAGGTCATGGAGTTTTTTATGAAAGTTTACGGATACAGGGGCAA |
| | | ACACCTTGGAGGGTCTCGGAAGCCTGATGGCGCAATTTATACCGTGG |
| | | GTAGCCCTATAGATTATGGAGTGATTGTGGATACAAAGGCTTACAGT |
| | | GGCGGCTATAATTTGCCTATCGGACAGGCCGATGAGATGGAAAGATA |
| | | CGTTGAAGAAAACCAAACACGAGATAAGCATCTGAACCCCAATGAAT |
| | | GGTGGAAAGTGTATCCTTCAAGCGTTACCGAGTTTAAGTTCCTCTTC |
| | | GTTTCTGGGCATTTCAAGGGCAACTACAAAGCTCAGCTTACAAGACT |
| | | CAACCACATAACCAATTGTGATGGAGCAGTCCTCAGCGTGGAAGAAC |
| | | TCCTTATTGGGGGTGAGATGATTAAAGCAGGGACCCTTACTCTTGAA |
| | | GAGGTTAGAAGAAAATTCAATAACGGAGAGATTAATTTTAGAAGT |
| 77 | Left ZFN | GCAGCCATGGCCGAACGCCCATTTCAATGTAGAATTTGTATGCAGAA |
| | (ZFN-L | TTTTTCACAATCAGGAAACCTGGCTAGACATATCAGAACACATACTG |
| | comprises ZFP- | GAGAAAAGCCCTTTGCTTGTGATATCTGTGGAAGGAAATTCGCCCTG |
| | L and FokI) | AAACAAAACCTCTGTATGCACACAAAGATCCACACCGGCGAAAAGCC |
| | (na) | TTTCCAGTGTAGGATATGCATGCAAAAATTCGCCTGGCAGTCCAATC |
| | Codon | TGCAGAACCATACCAAAATTCATACTGGTGAAAAGCCATTTCAGTGC |
| | diversified | AGAATATGTATGAGAAACTTTAGCACTTCAGGAAATCTCACAAGACA |
| | Version 6 | TATAAGAACACATACAGGGGAAAAACCTTTTGCTTGCGATATCTGCG |
| | | GCAGGAAATTCGCTCGGAGAAGTCATCTCACAAGCCATACAAAAATC |
| | | CACCTGCGAGGAAGCCAGCTGGTCAAGTCTGAACTGGAAGAAAAAAA |
| | | AAGCGAACTGCGGCATAAACTCAAATACGTCCCACATGAATACATTG |
| | | AGCTCATCGAAATTGCTAGAAACTCTACTCAAGATAGGATATTGGAG |
| | | ATGAAGGTAATGGAATTCTTCATGAAGGTTTATGGATATAGAGGAAA |
| | | ACATCTTGGAGGCAGTAGGAAACCCGATGGCGCTATCTACACCGTAG |
| | | GGAGTCCAATCGACTACGGCGTGATTGTTGACACCAAAGCCTATTCT |
| | | GGAGGGTATAATCTCCCAATTGGACAGGCAGATGAGATGGAAAGATA |
| | | TGTAGAAGAAAATCAGACAAGAGATAAGCACCTTAACCCTAACGAGT |
| | | GGTGGAAAGTGTACCCAAGCAGTGTTACTGAATTTAAATTTCTTTTT |
| | | GTATCAGGACACTTTAAAGGCAATTACAAAGCACAACTGACCAGACT |
| | | CAATCACATTACCAATTGCGACGGAGCCGTACTGAGCGTGGAGGAGT |
| | | TGCTGATCGGAGGCGAAATGATTAAAGCTGGCACTCTGACCCTGGAA |
| | | GAAGTAAGAAGAAAGTTCAATAATGGAGAAATAAACTTTCGCTCC |
| 78 | Right ZFN | GCCGCTATGGCTGAGAGGCCCTTCCAGTGTCGAATCTGCATGCGTAA |
| | (ZFN-R | CTTCAGTCAGTCCTCCGACCTGTCCCGCCACATCCGCACCCACACCG |
| | comprises ZFP- | GCGAGAAGCCTTTTGCCTGTGACATTTGTGGGAGGAAATTTGCCCTG |
| | R and FokI) | AAGCACAACCTGCTGACCCATACCAAGATACACACGGGCGAGAAGCC |
| | (na) | CTTCCAGTGTCGAATCTGCATGCAGAACTTCAGTGACCAGTCCAACC |
| | Not diversified | TGCGCGCCCACATCCGCACCCACACCGGCGAGAAGCCTTTTGCCTGT |
| | | GACATTTGTGGGAGGAAATTTGCCCGCAACTTCTCCCTGACCATGCA |
| | | TACCAAGATACACACCGGAGAGCGCGGCTTCCAGTGTCGAATCTGCA |
| | | TGCGTAACTTCAGTCTGCGCCACGACCTGGAGCGCCACATCCGCACC |
| | | CACACCGGCGAGAAGCCTTTTGCCTGTGACATTTGTGGGAGGAAATT |
| | | TGCCCACCGCTCCAACCTGAACAAGCATACCAAGATACACCTGCGGG |
| | | GATCCCAGCTGGTGAAGAGCGAGCTGGAGGAGAAGAAGTCCGAGCTG |
| | | CGGCACAAGCTGAAGTACGTGCCCCACGAGTACATCGAGCTGATCGA |
| | | GATCGCCAGGAACAGCACCCAGGACCGCATCCTGGAGATGAAGGTGA |
| | | TGGAGTTCTTCATGAAGGTGTACGGCTACAGGGGAAAGCACCTGGGC |
| | | GGAAGCAGAAAGCCTGACGGCGCCATCTATACAGTGGGCAGCCCCAT |
| | | CGATTACGGCGTGATCGTGGACACAAAGGCCTACAGCGGCGGCTACA |
| | | ATCTGAGCATCGGCCAGGCCGACGAGATGCAGAGATACGTGAAGGAG |
| | | AACCAGACCCGGAATAAGCACATCAACCCCAACGAGTGGTGGAAGGT |
| | | GTACCCTAGCAGCGTGACCGAGTTCAAGTTCCTGTTCGTGAGCGGCC |
| | | ACTTCAAGGGCAACTACAAGGCCCAGCTGACCAGGCTGAACCGCAAA |
| | | ACCAACTGCAATGGCGCCGTGCTGAGCGTGGAGGAGCTGCTGATCGG |
| | | CGGCGAGATGATCAAAGCCGGCACCCTGACACTGGAGGAGGTGCGGC |
| | | GCAAGTTCAACAACGGCGAGATCAACTTC |
| 79 | Right ZFN | GCTGCTATGGCTGAAAGACCTTTTCAATGTCGAATCTGCATGAGGAA |
| | (ZFN-R | TTTTAGTCAGTCATCCGACCTGAGCAGACACATTCGAACCCATACTG |
| | comprises ZFP- | GTGAAAAGCCATTTGCTTGCGATATATGTGGGAGAAAATTTGCGTTG |
| | R and FokI) | AAACACAATCTGCTGACCCATACCAAGATTCATACCGGAGAAAAACC |
| | (na) | ATTCCAATGCCGCATTTGTATGCAGAACTTTAGTGACCAGTCAAATC |
| | Codon | TCCGCGCTCACATTCGAACCCACACTGGCGAAAAACCCTTTGCTTGT |
| | diversified | GACATTTGCGGTCGGAAGTTTGCCCGAAATTTTTCTCTGACAATGCA |
| | Version 1 | CACAAAAATCCACACCGGGGAACGCGGCTTTCAATGTAGGATCTGTA |
| | | TGAGAAATTTTAGCCTTAGACATGATTTGGAACGACATATCAGGACC |
| | | CATACAGGCGAGAAACCATTTGCGTGCGATATTTGTGGCAGGAAATT |
| | | CGCACATAGAAGTAATCTGAACAAGCATACAAAAATTCATCTCAGAG |
| | | GAAGTCAGCTGGTCAAAAGTGAACTGGAGGAAAAAAAGAGCGAACTG |
| | | AGACACAAACTGAAGTACGTGCCACACGAATATATTGAGCTGATTGA |
| | | GATCGCGAGGAACTCAACACAGGACCGCATTCTGGAGATGAAAGTGA |
| | | TGGAGTTTTTCATGAAAGTATATGGATATAGAGGAAAACACCTTGGG |
| | | GGTAGCCGAAAGCCGGACGGGGCGATCTACACTGTGGGGTCACCAAT |
| | | TGATTATGGCGTAATTGTCGATACCAAAGCCTACAGTGGGGGGTACA |
| | | ATCTGAGTATAGGACAGGCTGATGAAATGCAACGATACGTTAAGGAG |
| | | AATCAGACTAGGAATAAACATATCAATCCAAATGAATGGTGGAAAGT |
| | | CTATCCCAGCAGCGTGACAGAATTTAAATTTTTGTTTGTCAGTGGAC |
| | | ACTTCAAGGGAAATTATAAGGCCCAGCTGACTAGACTGAATAGGAAA |
| | | ACCAATTGTAATGGCGCAGTGCTTTCAGTGGAGGAACTGCTCATTGG |
| | | AGGTGAGATGATCAAGGCTGGAACCCTGACGCTGGAGGAGGTGCGGA |
| | | GGAAGTTTAACAATGGAGAAATTAACTTT |
| 80 | Right ZFN | GCTGCCATGGCCGAGAGACCATTTCAATGTCGGATTTGCATGCGCAA |
| | (ZFN-R | TTTTTCCCAGTCCTCTGACCTTAGCCGGCATATTCGGACACACACAG |
| | comprises ZFP- | GTGAAAAACCCTTCGCATGCGACATTTGCGGAAGAAAATTCGCTCTG |
| | R and FokI) | AAACACAACCTGCTTACCCATACAAAGATCCACACCGGCGAGAAACC |
| | (na) | GTTTCAATGCCGAATCTGTATGCAAAATTTTAGTGATCAAAGTAATC |
| | Codon | TGAGAGCACATATTAGGACTCACACGGGCGAGAAGCCATTTGCGTGT |
| | diversified | GATATCTGCGGCCGAAAATTCGCCCGGAATTTCTCTCTGACAATGCA |
| | Version 2 | CACCAAAATCCACACTGGGGAACGAGGCTTTCAATGTAGAATATGTA |
| | | TGCGGAATTTCAGTCTGAGGCACGACCTGGAGCGGCACATCAGAACT |
| | | CACACCGGAGAAAAACCATTCGCTTGTGATATTTGCGGGAGGAAGTT |
| | | CGCCCATAGGAGCAATCTCAATAAACACACCAAAATACATCTTCGGG |
| | | GTTCTCAACTGGTGAAATCCGAACTGGAAGAAAAGAAATCAGAATTG |
| | | CGGCATAAACTGAAGTATGTGCCCCATGAGTAGATAGAACTGATCGA |
| | | GATCGCAAGGAACTCTAGCCAGGACAGAATACTTGAAATGAAGGTCA |
| | | TGGAATTTTTTATGAAAGTGTACGGCTACAGAGGAAAACATTTGGGA |
| | | GGCAGTCGAAAACCAGATGGCGCAATCTATACAGTCGGGTCCCCCAT |
| | | AGATTACGGAGTGATTGTCGACACAAAAGCCTATTCCGGAGGATATA |
| | | ACCTTAGTATCGGCCAGGCCGACGAGATGCAACGCTATGTGAAAGAA |
| | | AACCAAACAAGAAATAAACATATCAATCCAAACGAGTGGTGGAAGGT |
| | | ATATCCAAGCAGTGTCACAGAATTCAAATTCCTCTTCGTGAGTGGGC |
| | | ACTTTAAAGGCAACTACAAAGCTCAATTGACCAGGCTCAATCGGAAA |
| | | ACTAATTGCAATGGCGCAGTCCTTAGCGTCGAAGAATTGCTGATTGG |
| | | CGGGGAAATGATTAAAGCAGGAACTTTGACCTTGGAGGAAGTACGGA |
| | | GAAAGTTTAACAACGGCGAGATTAATTTT |
| 81 | Right ZFN | GCCGCCATGGCCGAGCGCCCCTTCCAATGCCGCATATGCATGAGAAA |
| | (ZFN-R | TTTCAGCCAAAGTAGCGACCTGTCACGACACATTAGAACTCATACGG |
| | comprises ZFP- | GGGAGAAGCCATTTGCTTGCGATATTTGTGGCAGAAAATTCGCACTC |
| | R and FokI) | AAACACAACCTGCTCACACACACCAAGATACACACGGGAGAGAAGCC |
| | (na) | CTTCCAATGTAGAATATGTATGCAAAATTTCAGCGACCAAAGTAATT |
| | Codon | TGAGAGCGCATATTCGAACTCACACCGGCGAAAAACCATTTGCCTGC |
| | diversified | GATATTTGTGGGAGGAAATTTGCCAGGAATTTTTCACTCACCATGCA |
| | Version 3 | CACTAAGATCCACACTGGCGAGCGCGGCTTCCAATGCAGAATCTGTA |
| | | TGCGAAACTTCAGTCTGCGGCATGACCTGGAAAGACATATAAGAACC |
| | | CACACCGGAGAAAAACCCTTTGCCTGCGACATATGTGGTAGAAAATT |
| | | CGCACATCGGAGTAACCTTAACAAACATACAAAGATCCACTTGAGAG |
| | | GCAGTCAGCTGGTGAAATCTGAGCTGGAAGAGAAGAAATCTGAACTG |
| | | CGACATAAATTGAAGTACGTCCCACACGAGTAGATCGAGTTGATCGA |
| | | AATTGCCCGGAATAGCACCCAGGATAGAATATTGGAAATGAAAGTAA |
| | | TGGAGTTTTTTATGAAGGTTTATGGTTACAGAGGCAAGCACCTTGGA |
| | | GGAAGCAGGAAACCAGATGGGGCGATTTACACCGTTGGGAGTCCCAT |
| | | CGATTACGGAGTCATCGTGGACACAAAGGCCTATTCCGGAGGCTACA |
| | | ACCTCAGTATCGGGCAAGCCGATGAGATGCAGAGATATGTTAAAGAA |
| | | AATCAGACGCGAAACAAGCACATTAACCCAAACGAATGGTGGAAAGT |
| | | TTACCCTAGCTCAGTGACAGAATTTAAGTTTCTGTTTGTCAGCGGCC |
| | | ACTTCAAGGGGAATTATAAAGCACAACTGACCCGCCTGAACCGAAAA |
| | | ACCAACTGTAACGGTGCTGTGCTGAGTGTCGAAGAGTTGCTTATCGG |
| | | AGGAGAGATGATAAAGGCCGGCACACTGACGCTTGAAGAGGTACGGC |
| | | GAAAATTCAATAACGGAGAGATTAATTTT |
| 82 | Right ZFN | GCCGCTATGGCTGAAAGACCTTTCCAGTGTAGGATTTGCATGAGAAA |
| | (ZFN-R | TTTTTCCCAATCATCCGACCTTTCAAGGCATATTAGGACACACACCG |
| | comprises ZFP- | GGGAAAAGCCATTTGCTTGTGATATCTGCGGGCGCAAATTTGCTCTT |
| | R and FokI) | AAGCACAATCTTCTTACCCACACCAAAATTCATACAGGAGAAAAACC |
| | (na) | TTTTCAATGTAGAATCTGCATGCAAAACTTTTCCGATCAGTCAAATC |
| | Codon | TTAGAGCTCATATCAGAACCCATACCGGGGAGAAACCCTTTGCCTGC |
| | diversified | GACATATGCGGAAGAAAATTTGCTAGGAACTTTAGTCTGACCATGCA |
| | Version 4 | TACCAAAATTCATACCGGCGAACGCGGTTTCCAGTGCAGGATTTGTA |
| | | TGAGAAATTTCTCACTGCGGCATGATCTTGAAAGACACATACGAACT |
| | | CATACCGGAGAAAAGCCATTCGCTTGCGATATTTGTGGTAGAAAATT |
| | | TGCCCACAGGTCTAACCTTAATAAGCACACCAAGATTCATCTCAGAG |
| | | GATCTCAGCTGGTCAAATCAGAACTTGAAGAGAAAAAAAGCGAACTG |
| | | AGACATAAACTGAAGTACGTGCCTCATGAATACATAGAGCTCATTGA |
| | | AATAGCTAGGAATAGTACACAGGACAGGATACTTGAAATGAAGGTAA |
| | | TGGAATTTTTCATGAAGGTTTATGGATACCGGGGGAAACATCTCGGG |
| | | GGCAGCAGAAAACCAGACGGAGCAATTTATACTGTCGGGAGTCCTAT |
| | | AGATTATGGCGTTATCGTCGATACAAAGGCCTATTCCGGTGGGTACA |
| | | ACCTCTCAATTGGTCAGGCTGATGAGATGCAAAGATACGTCAAAGAA |
| | | AACCAAACCAGAAATAAACATATAAATCCCAATGAATGGTGGAAAGT |
| | | ATACCCAAGTTCCGTGACTGAATTCAAGTTCCTTTTCGTGTCTGGCC |
| | | ACTTTAAAGGAAATTATAAAGCACAATTGACTAGACTGAATAGAAAA |
| | | ACAAACTGTAACGGCGCAGTGCTGTCAGTGGAAGAACTGCTCATAGG |
| | | TGGAGAGATGATCAAGGCCGGGACACTTAGTCTTGAGGAAGTTAGAA |
| | | GGAAGTTCAACAACGGCGAAATCAACTTT |
| 83 | Right ZFN | GCAGCTATGGCCGAACGCCCTTTTCAATGCAGAATATGTATGCGAAA |
| | (ZFN-R | CTTCTCCCAAAGCTCTGATCTGTCAAGGCACATACGGACACACACCG |
| | comprises ZFP- | GCGAAAAACCCTTTGCATGTGACATTTGTGGAAGAAAATTCGCACTT |
| | R and FokI) | AAACACAATCTCCTGACTCATACAAAAATACATACAGGCGAAAAACC |
| | Codon | TTTCCAGTGCAGAATCTGTATGCAGAACTTTTCCGACCAATCCAATC |
| | diversified | TTCGCGCCCACATTAGAACTCACACAGGGGAGAAACCTTTCGCTTGC |
| | Version 5 | GACATATGCGGAAGAAAATTTGCCAGAAATTTTTCACTTACAATGCA |
| | | CACAAAAATACATACTGGGGAAAGAGGGTTTCAATGTCGAATCTGTA |
| | | TGAGAAATTTCAGTCTGCGCCATGATCTGGAGAGACATATAAGAACA |
| | | CACACAGGAGAGAAACCTTTTGCTTGTGACATATGCGGCCGAAAGTT |
| | | TGCTCATAGATCTAATCTTAACAAACATACAAAGATCCATCTTCGGG |
| | | GTTCACAACTGGTCAAGTCAGAATTGGAAGAGAAAAAATCTGAGCTG |
| | | AGGCACAAATTGAAATACGTTCCTCACGAGTATATTGAACTTATCGA |
| | | GATAGCCCGCAATAGTAGACAAGATAGAATCTTGGAGATGAAAGTTA |
| | | TGGAATTCTTTATGAAAGTCTATGGCTATAGGGGAAAACACCTGGGG |
| | | GGTAGCAGGAAACCTGATGGAGCTATCTATACCGTAGGATCACCTAT |
| | | TGATTATGGAGTAATTGTGGACACTAAGGCATATTCCGGAGGATATA |
| | | ATTTGAGTATTGGTCAGGCCGACGAAATGCAACGATACGTGAAGGAA |
| | | AATCAGACCCGCAACAAACACATTAATCCCAATGAATGGTGGAAGGT |
| | | ATACCCTAGTAGCGTTACAGAGTTTAAATTCCTTTTCGTCAGCGGCC |
| | | ACTTTAAAGGAAATTATAAAGCACAACTCACCAGACTTAATCGAAAA |
| | | ACTAACTGTAACGGCGCCGTACTGTCAGTGGAGGAGCTGCTCATTGG |
| | | AGGCGAGATGATCAAGGCCGGTACTCTCACACTGGAAGAAGTTAGAA |
| | | GAAAGTTCAACAACGGGGAAATTAATTTC |
| 84 | Right ZFN | GCTGCTATGGCTGAGAGACCTTTCCAATGTAGGATCTGTATGCGAAA |
| | (ZFN-R | CTTCTCCCAGAGCTCCGACCTGAGTCGCCATATAAGAACCCATACCG |
| | comprises ZFP- | GAGAAAAACCATTTGCTTGTGACATTTGTGGCAGAAAGTTCGCTCTT |
| | R and FokI) | AAACACAACCTGCTTACACATACTAAAATACACACAGGGGAGAAACC |
| | (na) | CTTTCAATGCCGGATCTGTATGCAAAACTTTAGCGATCAATCAAACT |
| | Codon | TGCGAGCCCATATCCGCACTCACACCGGCGAGAAGCCTTTTGCATGC |
| | diversified | GATATATGTGGACGGAAATTTGCTAGAAACTTCTCATTGACCATGCA |
| | Version 6 | TACAAAAATACACACCGGGGAACGAGGATTTCAATGTCGAATTTGTA |
| | | TGAGAAATTTTAGCCTTAGGCACGACTTGGAACGGCACATAAGAACC |
| | | CACACCGGAGAGAAGCCTTTTGCTTGTGATATTTGCGGCAGAAAGTT |
| | | CGCCCATCGCAGCAATCTTAACAAGCACACCAAGATTCATTTGAGAG |
| | | GTTCCCAGCTGGTCAAAAGCGAACTTGAAGAAAAGAAATCCGAGCTT |
| | | AGACACAAACTGAAATACGTGCCTCACGAGTATATTGAGCTGATTGA |
| | | AATAGCAAGGAATTCAACACAAGACAGGATCCTCGAAATGAAGGTTA |
| | | TGGAGTTTTTCATGAAAGTTTACGGCTACAGAGGGAAGCATCTGGGC |
| | | GGATCAAGAAAACCAGACGGCGCAATCTACACAGTTGGATCCCCAAT |
| | | AGATTACGGAGTGATTGTTGACACCAAGGCTTATTCAGGAGGTTACA |
| | | ATCTGTCCATTGGTCAGGCCGATGAAATGCAAAGATATGTTAAGGAA |
| | | AATCAAACTCGAAACAAACACATTAATCCAAACGAATGGTGGAAAGT |
| | | ATATCCAAGCTCCGTCACTGAATTTAAATTTTTGTTTGTATCCGGAC |
| | | ATTTTAAGGGCAACTATAAGGCTCAACTGACCAGACTGAATAGGAAG |
| | | ACCAATTGTAACGGAGCTGTACTCAGCGTGGAAGAACTGCTTATTGG |
| | | AGGCGAAATGATTAAGGCTGGCACACTTACACTCGAAGAAGTTAGAA |
| | | GAAAATTCAACAATGGTGAGATAAACTTC |
| 85 | Right ZFN- | GATTATAAAGATCATGACGGGGACTATAAGGATCACGACATAGACTA |
| | T2A-Left ZFN | CAAAGACGATGATGACAAAATGGCGCCTAAAAAGAAACGAAAAGTGG |
| | with N-terminal | GCATTCACGGCGTACCTGCTGCTATGGCTGAAAGACCTTTTCAATGT |
| | modifications | CGAATCTGCATGAGGAATTTTAGTCAGTCATCCGACCTGAGCAGACA |
| | (comprising | CATTCGAACCCATACTGGTGAAAAGCCATTTGCTTGCGATATATGTG |
| | 3xFLAG, NLS, | GGAGAAAATTTGCGTTGAAACACAATCTGCTGACCCATACCAAGATT |
| | ZFP-R, FokI, | CATACCGGAGAAAAACCATTCCAATGCCGCATTTGTATGCAGAACTT |
| | T2A, 3xFLAG, | TAGTGACCAGTCAAATCTCCGCGCTCACATTCGAACCCACACTGGCG |
| | NLS, ZFP-L, | AAAAACCCTTTGCTTGTGACATTTGCGGTCGGAAGTTTGCCCGAAAT |
| | and FokI)(na) | TTTTCTCTGACAATGCACACAAAAATCCACACCGGGGAACGCGGCTT |
| | ZFN-R | TCAATGTAGGATCTGTATGAGAAATTTTAGCCTTAGACATGATTTGG |
| | Codon | AACGACATATCAGGACCCATACAGGCGAGAAACCATTTGCGTGCGAT |
| | diversified | ATTTGTGGCAGGAAATTCGCACATAGAAGTAATCTGAACAAGCATAC |
| | Version 1 | AAAAATTCATCTCAGAGGAAGTCAGCTGGTCAAAAGTGAACTGGAGG |
| | ZFN-L | AAAAAAAGAGCGAACTGAGACACAAACTGAAGTACGTGCCACACGAA |
| | Not diversified | TATATTGAGCTGATTGAGATCGCGAGGAACTCAACACAGGACCGCAT |
| | | TCTGGAGATGAAAGTGATGGAGTTTTTCATGAAAGTATATGGATATA |
| | | GAGGAAAACACCTTGGGGGTAGCCGAAAGCCGGACGGGGCGATCTAC |
| | | ACTGTGGGGTCACCAATTGATTATGGCGTAATTGTCGATACCAAAGC |
| | | CTACAGTGGGGGGTACAATCTGAGTATAGGACAGGCTGATGAAATGC |
| | | AACGATACGTTAAGGAGAATCAGACTAGGAATAAACATATCAATCCA |
| | | AATGAATGGTGGAAAGTCTATCCCAGCAGCGTGACAGAATTTAAATT |
| | | TTTGTTTGTCAGTGGACACTTCAAGGGAAATTATAAGGCCCAGCTGA |
| | | CTAGACTGAATAGGAAAACCAATTGTAATGGCGCAGTGCTTTCAGTG |
| | | GAGGAACTGCTCATTGGAGGTGAGATGATCAAGGCTGGAACCCTGAC |
| | | GCTGGAGGAGGTGCGGAGGAAGTTTAACAATGGAGAAATTAACTTTG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGACATCGATTACAAGGATGACGATGACAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCCAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 86 | Right ZFN- | GATTATAAAGACCATGATGGTGATTACAAGGACCATGACATCGATTA |
| | T2A-Left ZFN | TAAAGACGACGACGACAAAATGGCCCCTAAGAAAAAGAGAAAAGTCG |
| | with N-terminal | GAATCCACGGTGTCCCAGCTGCCATGGCCGAGAGACCATTTCAATGT |
| | modifications | CGGATTTGCATGCGCAATTTTTCCCAGTCCTCTGACCTTAGCCGGCA |
| | (comprising | TATTCGGACACACACAGGTGAAAAACCCTTCGCATGCGACATTTGCG |
| | 3xFLAG, NLS, | GAAGAAAATTCGCTCTGAAACACAACCTGCTTACCCATACAAAGATC |
| | ZFP-R, FokI, | CACACCGGCGAGAAACCGTTTCAATGCCGAATCTGTATGCAAAATTT |
| | T2A, 3xFLAG, | TAGTGATCAAAGTAATCTGAGAGCACATATTAGGACTCACACGGGCG |
| | NLS, ZFP-L, | AGAAGCCATTTGCGTGTGATATCTGCGGCCGAAAATTCGCCCGGAAT |
| | and FokI)(na) | TTCTCTCTGACAATGCACACCAAAATCCACACTGGGGAACGAGGCTT |
| | ZFN-R | TCAATGTAGAATATGTATGCGGAATTTCAGTCTGAGGCACGACCTGG |
| | Codon | AGCGGCACATCAGAACTCACACCGGAGAAAAACCATTCGCTTGTGAT |
| | diversified | ATTTGCGGGAGGAAGTTCGCCCATAGGAGCAATCTCAATAAACACAC |
| | Version 2 | CAAAATACATCTTCGGGGTTCTCAACTGGTGAAATCCGAACTGGAAG |
| | ZFN-L | AAAAGAAATCAGAATTGCGGCATAAACTGAAGTATGTGCCCCATGAG |
| | Not diversified | TAGATAGAACTGATCGAGATCGCAAGGAACTCTACCCAGGACAGAAT |
| | | ACTTGAAATGAAGGTCATGGAATTTTTTATGAAAGTGTACGGCTACA |
| | | GAGGAAAACATTTGGGAGGCAGTCGAAAACCAGATGGCGCAATCTAT |
| | | ACAGTCGGGTCCCCCATAGATTACGGAGTGATTGTCGACACAAAAGC |
| | | CTATTCCGGAGGATATAACCTTAGTATCGGCCAGGCCGACGAGATGC |
| | | AACGCTATGTGAAAGAAAACCAAACAAGAAATAAACATATCAATCCA |
| | | AACGAGTGGTGGAAGGTATATCCAAGCAGTGTCACAGAATTCAAATT |
| | | CCTCTTCGTGAGTGGGCACTTTAAAGGCAACTACAAAGCTCAATTGA |
| | | CCAGGCTCAATCGGAAAACTAATTGCAATGGCGCAGTCCTTAGCGTC |
| | | GAAGAATTGCTGATTGGCGGGGAAATGATTAAAGCAGGAACTTTGAC |
| | | CTTGGAGGAAGTACGGAGAAAGTTTAACAACGGCGAGATTAATTTTG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGACATCGATTACAAGGATGACGATGACAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCCAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 87 | Right ZFN- | GATTATAAGGATCATGATGGAGACTATAAGGATCATGACATAGATTA |
| | T2A-Left ZFN | CAAAGATGACGATGACAAGATGGCACCCAAGAAGAAAAGAAAAGTAG |
| | with N-terminal | GAATTCACGGAGTCCCTGCCGCCATGGCCGAGCGCCCCTTCCAATGC |
| | modifications | CGCATATGCATGAGAAATTTCAGCCAAAGTAGCGACCTGTCACGACA |
| | (comprising | CATTAGAACTCATACGGGGGAGAAGCCATTTGCTTGCGATATTTGTG |
| | 3xFLAG, NLS, | GCAGAAAATTCGCACTCAAACACAACCTGCTCACACACACCAAGATA |
| | ZFP-R, FokI, | CACACGGGAGAGAAGCCCTTCCAATGTAGAATATGTATGCAAAATTT |
| | T2A, 3xFLAG, | CAGCGACCAAAGTAATTTGAGAGCGCATATTCGAACTCACACCGGCG |
| | NLS, ZFP-L, | AAAAACCATTTGCCTGCGATATTTGTGGGAGGAAATTTGCCAGGAAT |
| | and FokI)(na) | TTTTCACTCACCATGCACACTAAGATCCACACTGGCGAGCGCGGCTT |
| | ZFN-R | CCAATGCAGAATCTGTATGCGAAACTTCAGTCTGCGGCATGACCTGG |
| | Codon | AAAGACATATAAGAACCCACACCGGAGAAAAACCCTTTGCCTGCGAC |
| | diversified | ATATGTGGTAGAAAATTCGCACATCGGAGTAACCTTAACAAACATAC |
| | Version 3 | AAAGATCCACTTGAGAGGCAGTCAGCTGGTGAAATCTGAGCTGGAAG |
| | ZFN-L | AGAAGAAATCTGAACTGCGACATAAATTGAAGTACGTCCCACACGAG |
| | Not diversified | TACATCGAGTTGATCGAAATTGCCCGGAATAGCACCCAGGATAGAAT |
| | | ATTGGAAATGAAAGTAATGGAGTTTTTTATGAAGGTTTATGGTTACA |
| | | GAGGCAAGCACCTTGGAGGAAGCAGGAAACCAGATGGGGCGATTTAC |
| | | ACCGTTGGGAGTCCCATCGATTACGGAGTCATCGTGGACACAAAGGC |
| | | CTATTCCGGAGGCTACAACCTCAGTATCGGGCAAGCCGATGAGATGC |
| | | AGAGATATGTTAAAGAAAATCAGACGCGAAACAAGCACATTAACCCA |
| | | AACGAATGGTGGAAAGTTTACCCTAGCTCAGTGACAGAATTTAAGTT |
| | | TCTGTTTGTCAGCGGCCACTTCAAGGGGAATTATAAAGCACAACTGA |
| | | CCCGCCTGAACCGAAAAACCAACTGTAACGGTGCTGTGCTGAGTGTC |
| | | GAAGAGTTGCTTATCGGAGGAGAGATGATAAAGGCCGGCACACTGAC |
| | | GCTTGAAGAGGTACGGCGAAAATTCAATAACGGAGAGATTAATTTTG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGACATCGATTACAAGGATGACGATGACAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCCAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 88 | Right ZFN- | GACTACAAAGATCATGATGGCGACTACAAAGATCATGATATAGATTA |
| | T2A-Left ZFN | CAAAGACGATGACGACAAAATGGCTCCAAAAAAAAAACGCAAGGTTG |
| | with N-terminal | GAATACACGGTGTACCTGCCGCTATGGCTGAAAGACCTTTCCAGTGT |
| | modifications | AGGATTTGCATGAGAAATTTTTCCCAATCATCCGACCTTTCAAGGCA |
| | (comprising | TATTAGGACACACACCGGGGAAAAGCCATTTGCTTGTGATATCTGCG |
| | 3xFLAG, NLS, | GGCGCAAATTTGCTCTTAAGCACAATCTTCTTACCCACACCAAAATT |
| | ZFP-R, FokI, | CATACAGGAGAAAAACCTTTTCAATGTAGAATCTGCATGCAAAACTT |
| | T2A, 3xFLAG, | TTCCGATCAGTCAAATCTTAGAGCTCATATCAGAACCCATACCGGGG |
| | NLS, ZFP-L, | AGAAACCCTTTGCCTGCGACATATGCGGAAGAAAATTTGCTAGGAAC |
| | and FokI)(na) | TTTAGTCTGACCATGCATACCAAAATTCATACCGGCGAACGCGGTTT |
| | ZFN-R | CCAGTGCAGGATTTGTATGAGAAATTTCTCACTGCGGCATGATCTTG |
| | Codon | AAAGACACATACGAACTCATACCGGAGAAAAGCCATTCGCTTGCGAT |
| | diversified | ATTTGTGGTAGAAAATTTGCCCACAGGTCTAACCTTAATAAGCACAC |
| | Version 4 | CAAGATTCATCTCAGAGGATCTCAGCTGGTCAAATCAGAACTTGAAG |
| | ZFN-L | AGAAAAAAAGCGAACTGAGACATAAACTGAAGTACGTGCCTCATGAA |
| | Not diversified | TACATAGAGCTCATTGAAATAGCTAGGAATAGTACACAGGACAGGAT |
| | | ACTTGAAATGAAGGTAATGGAATTTTTCATGAAGGTTTATGGATACC |
| | | GGGGGAAACATCTCGGGGGCAGCAGAAAACCAGACGGAGCAATTTAT |
| | | ACTGTCGGGAGTCCTATAGATTATGGCGTTATCGTCGATACAAAGGC |
| | | CTATTCCGGTGGGTACAACCTCTCAATTGGTCAGGCTGATGAGATGC |
| | | AAAGATACGTCAAAGAAAACCAAACCAGAAATAAACATATAAATCCC |
| | | AATGAATGGTGGAAAGTATACCCAAGTTCCGTGACTGAATTCAAGTT |
| | | CCTTTTCGTGTCTGGCCACTTTAAAGGAAATTATAAAGCACAATTGA |
| | | CTAGACTGAATAGAAAAACAAACTGTAACGGCGCAGTGCTGTCAGTG |
| | | GAAGAACTGCTCATAGGTGGAGAGATGATCAAGGCCGGGACACTTAC |
| | | TCTTGAGGAAGTTAGAAGGAAGTTCAACAACGGCGAAATCAACTTTG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGACATCGATTACAAGGATGACGATGACAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCCAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 89 | Right ZFN- | GATTACAAAGACCATGATGGCGACTATAAAGACCATGACATCGACTA |
| | T2A-Left ZFN | CAAGGATGATGATGATAAAATGGCTCCAAAGAAAAAGAGGAAGGTGG |
| | with N-terminal | GAATACATGGAGTACCAGCAGCTATGGCCGAACGCCCTTTTCAATGC |
| | modifications | AGAATATGTATGCGAAACTTCTCCCAAAGCTCTGATCTGTCAAGGCA |
| | (comprising | CATACGGACACACACCGGCGAAAAACCCTTTGCATGTGACATTTGTG |
| | 3xFLAG, NLS, | GAAGAAAATTCGCACTTAAACACAATCTCCTGACTCATACAAAAATA |
| | ZFP-R, FokI, | CATACAGGCGAAAAACCTTTCCAGTGCAGAATCTGTATGCAGAACTT |
| | T2A, 3xFLAG, | TTCCGACCAATCCAATCTTCGCGCCCACATTAGAACTCACACAGGGG |
| | NLS, ZFP-L, | AGAAACCTTTCGCTTGCGACATATGCGGAAGAAAATTTGCCAGAAAT |
| | and FokI)(na) | TTTTCACTTACAATGCACACAAAAATACATACTGGGGAAAGAGGGTT |
| | ZFN-R | TCAATGTCGAATCTGTATGAGAAATTTCAGTCTGCGCCATGATCTGG |
| | Codon | AGAGACATATAAGAACACACACAGGAGAGAAACCTTTTGCTTGTGAC |
| | diversified | ATATGCGGCCGAAAGTTTGCTCATAGATCTAATCTTAACAAACATAC |
| | Version 5 | AAAGATCCATCTTCGGGGTTCACAACTGGTCAAGTCAGAATTGGAAG |
| | ZFN-L | AGAAAAAATCTGAGCTGAGGCACAAATTGAAATACGTTCCTCACGAG |
| | Not diversified | TATATTGAACTTATCGAGATAGCCCGCAATAGTAGACAAGATAGAAT |
| | | CTTGGAGATGAAAGTTATGGAATTCTTTATGAAAGTCTATGGCTATA |
| | | GGGGAAAACACCTGGGGGGTAGCAGGAAACCTGATGGAGCTATCTAT |
| | | ACCGTAGGATCACCTATTGATTATGGAGTAATTGTGGACACTAAGGC |
| | | ATATTCCGGAGGATATAATTTGAGTATTGGTCAGGCCGACGAAATGC |
| | | AACGATACGTGAAGGAAAATCAGACCCGCAACAAACACATTAATCCC |
| | | AATGAATGGTGGAAGGTATACCCTAGTAGCGTTACAGAGTTTAAATT |
| | | CCTTTTCGTCAGCGGCCACTTTAAAGGAAATTATAAAGCACAACTCA |
| | | CCAGACTTAATCGAAAAACTAACTGTAACGGCGCCGTACTGTCAGTG |
| | | GAGGAGCTGCTCATTGGAGGCGAGATGATCAAGGCCGGTACTCTCAC |
| | | ACTGGAAGAAGTTAGAAGAAAGTTCAACAACGGGGAAATTAATTTCG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGACATCGATTACAAGGATGACGATGACAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCCAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 90 | Right ZFN- | GACTACAAGGACCACGACGGAGACTATAAAGACCATGATATAGATTA |
| | T2A-Left ZFN | CAAGGACGATGACGATAAAATGGCACCCAAAAAGAAAAGAAAGGTGG |
| | with N-terminal | GTATTCACGGAGTTCCCGCTGCTATGGCTGAGAGACCTTTCCAATGT |
| | modifications | AGGATCTGTATGCGAAACTTCTCCCAGAGCTCCGACCTGAGTCGCCA |
| | (comprising | TATAAGAACCCATACCGGAGAAAAACCATTTGCTTGTGACATTTGTG |
| | 3xFLAG, NLS, | GCAGAAAGTTCGCTCTTAAACACAACCTGCTTACACATACTAAAATA |
| | ZFP-R, FokI, | CACACAGGGGAGAAACCCTTTCAATGCCGGATCTGTATGCAAAACTT |
| | T2A, 3xFLAG, | TAGCGATCAATCAAACTTGCGAGCCCATATCCGCACTCACACCGGCG |
| | NLS, ZFP-L, | AGAAGCCTTTTGCATGCGATATATGTGGACGGAAATTTGCTAGAAAC |
| | and FokI)(na) | TTCTCATTGACCATGCATACAAAAATACACACCGGGGAACGAGGATT |
| | ZFN-R | TCAATGTCGAATTTGTATGAGAAATTTTAGCCTTAGGCACGACTTGG |
| | Codon | AACGGCACATAAGAACCCACACCGGAGAGAAGCCTTTTGCTTGTGAT |
| | diversified | ATTTGCGGCAGAAAGTTCGCCCATCGCAGCAATCTTAACAAGCACAC |
| | Version 6 | CAAGATTCATTTGAGAGGTTCCCAGCTGGTCAAAAGCGAACTTGAAG |
| | ZFN-L | AAAAGAAATCCGAGCTTAGACACAAACTGAAATACGTGCCTCACGAG |
| | Not diversified | TATATTGAGCTGATTGAAATAGCAAGGAATTCAACACAAGACAGGAT |
| | | CCTCGAAATGAAGGTTATGGAGTTTTTCATGAAAGTTTACGGCTACA |
| | | GAGGGAAGCATCTGGGCGGATCAAGAAAACCAGACGGCGCAATCTAC |
| | | ACAGTTGGATCCCCAATAGATTACGGAGTGATTGTTGACACCAAGGC |
| | | TTATTCAGGAGGTTACAATCTGTCCATTGGTCAGGCCGATGAAATGC |
| | | AAAGATATGTTAAGGAAAATCAAACTCGAAACAAACACATTAATCCA |
| | | AACGAATGGTGGAAAGTATATCCAAGCTCCGTCACTGAATTTAAATT |
| | | TTTGTTTGTATCCGGACATTTTAAGGGCAACTATAAGGCTCAACTGA |
| | | CCAGACTGAATAGGAAGACCAATTGTAACGGAGCTGTACTCAGCGTG |
| | | GAAGAACTGCTTATTGGAGGCGAAATGATTAAGGCTGGCACACTTAC |
| | | ACTCGAAGAAGTTAGAAGAAAATTCAACAATGGTGAGATAAACTTCG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGACATCGATTACAAGGATGACGATGACAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCCAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 91 | Right ZFN- | GACTACAAAGACCATGACGGTGATTATAAAGATCATGACATCGATTA |
| | T2A-Left ZFN | CAAGGATGACGATGACAAGATGGCCCCCAAGAAGAAGAGGAAGGTCG |
| | with N-terminal | GCATTCATGGGGTACCCGCCGCTATGGCTGAGAGGCCCTTCCAGTGT |
| | modifications | CGAATCTGCATGCGTAACTTCAGTCAGTCCTCCGACCTGTCCCGCCA |
| | (comprising | CATCCGCACCCACACCGGCGAGAAGCCTTTTGCCTGTGACATTTGTG |
| | 3xFLAG, NLS, | GGAGGAAATTTGCCCTGAAGCACAACCTGCTGACCCATACCAAGATA |
| | ZFP-R, FokI, | CACACGGGCGAGAAGCCCTTCCAGTGTCGAATCTGCATGCAGAACTT |
| | T2A, 3xFLAG, | CAGTGACCAGTCCAACCTGCGCGCCCACATCCGCACCCACACCGGCG |
| | NLS, ZFP-L, | AGAAGCCTTTTGCCTGTGACATTTGTGGGAGGAAATTTGCCCGCAAC |
| | and FokI)(na) | TTCTCCCTGACCATGCATACCAAGATACACACCGGAGAGCGCGGCTT |
| | ZFN-R | CCAGTGTCGAATCTGCATGCGTAACTTCAGTCTGCGCCACGACCTGG |
| | Not diversified | AGCGCCACATCCGCACCCACACCGGCGAGAAGCCTTTTGCCTGTGAC |
| | ZFN-L | ATTTGTGGGAGGAAATTTGCCCACCGCTCCAACCTGAACAAGCATAC |
| | Not diversified | CAAGATACACCTGCGGGGATCCCAGCTGGTGAAGAGCGAGCTGGAGG |
| | | AGAAGAAGTCCGAGCTGCGGCACAAGCTGAAGTACGTGCCCCACGAG |
| | | TACATCGAGCTGATCGAGATCGCCAGGAACAGCACCCAGGACCGCAT |
| | | CCTGGAGATGAAGGTGATGGAGTTCTTCATGAAGGTGTACGGCTACA |
| | | GGGGAAAGCACCTGGGCGGAAGCAGAAAGCCTGACGGCGCCATCTAT |
| | | ACAGTGGGCAGCCCCATCGATTACGGCGTGATCGTGGACACAAAGGC |
| | | CTACAGCGGCGGCTACAATCTGAGCATCGGCCAGGCCGACGAGATGC |
| | | AGAGATACGTGAAGGAGAACCAGACCCGGAATAAGCACATCAACCCC |
| | | AACGAGTGGTGGAAGGTGTACCCTAGCAGCGTGACCGAGTTCAAGTT |
| | | CCTGTTCGTGAGCGGCCACTTCAAGGGCAACTACAAGGCCCAGCTGA |
| | | CCAGGCTGAACCGCAAAACCAACTGCAATGGCGCCGTGCTGAGCGTG |
| | | GAGGAGCTGCTGATCGGCGGCGAGATGATCAAAGCCGGCACCCTGAC |
| | | ACTGGAGGAGGTGCGGCGCAAGTTCAACAACGGCGAGATCAACTTCG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGACATCGATTACAAGGATGACGATGACAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCCAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 92 | Left ZFN-T2A- | GATTACAAAGATCACGACGGAGATTAGAAAGATCACGACATTGACTA |
| | Right ZFN | TAAGGACGACGACGATAAAATGGCTCCAAAGAAGAAAAGAAAAGTGG |
| | with N-terminal | GGATCCATGGTGTACCCGCAGCAATGGCCGAACGACCCTTCCAATGC |
| | modifications | AGAATATGTATGCAGAATTTTTCTCAGAGCGGGAACCTGGCGAGGCA |
| | (comprising | CATAAGAACCCATACAGGAGAGAAGCCATTCGCATGCGATATTTGCG |
| | 3xFLAG, NLS, | GTAGAAAATTTGCACTCAAACAAAATCTCTGTATGCACACTAAAATC |
| | ZFP-L, FokI, | CATACAGGTGAAAAGCCTTTTCAGTGCAGGATTTGTATGCAAAAATT |
| | T2A, 3xFLAG, | TGCTTGGCAAAGTAACTTGCAGAACCACACAAAGATACACACAGGAG |
| | NLS, ZFP-R, | AGAAACCCTTCCAATGCCGAATCTGTATGCGCAACTTCAGTACATCC |
| | and FokI)(na) | GGAAATTTGACTAGACATATTAGGACCCACACCGGCGAGAAGCCATT |
| | ZFN-L | TGCCTGCGATATTTGTGGACGGAAATTCGCACGACGCAGCCATCTGA |
| | Codon | CCAGTCATACTAAGATTCATCTCCGCGGCAGCCAGCTTGTGAAGTCC |
| | diversified | GAACTGGAGGAAAAGAAGAGCGAACTGCGCCACAAATTGAAATACGT |
| | Version 1 | TCCGCATGAGTACATAGAGCTCATTGAAATCGCTAGAAACTCTACCC |
| | ZFN-R | AAGACAGGATACTGGAAATGAAAGTGATGGAATTTTTCATGAAAGTT |
| | Not diversified | TATGGTTATAGGGGCAAACATCTGGGTGGCTCTCGCAAGCCCGATGG |
| | | GGCCATTTATACTGTCGGCTCACCTATCGACTATGGCGTCATTGTGG |
| | | ATACCAAGGCTTATTCTGGAGGATACAACCTGCCCATCGGACAAGCA |
| | | GACGAAATGGAAAGATACGTCGAGGAGAATCAAACCCGAGACAAGCA |
| | | TCTGAACCCAAACGAGTGGTGGAAAGTGTACCCGAGCAGCGTTACTG |
| | | AGTTCAAATTTCTCTTTGTAAGCGGACATTTTAAAGGGAATTACAAA |
| | | GCACAACTGACTAGGCTGAACCATATAACCAACTGTGACGGGGCCGT |
| | | ATTGAGTGTGGAAGAGCTTCTGATTGGAGGAGAGATGATTAAGGCTG |
| | | GCACACTGACTCTCGAAGAAGTGAGGCGCAAATTCAATAACGGTGAA |
| | | ATCAACTTCCGGTCTGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGACCATGACGGTGATTATAAAGATCATGACATCGATTACAAG |
| | | GATGACGATGACAAGATGGCCCCCAAGAAGAAGAGGAAGGTCGGCAT |
| | | TCATGGGGTACCCGCCGCTATGGCTGAGAGGCCCTTCCAGTGTCGAA |
| | | TCTGCATGCGTAACTTCAGTCAGTCCTCCGACCTGTCCCGCCACATC |
| | | CGCACCCACACCGGCGAGAAGCCTTTTGCCTGTGACATTTGTGGGAG |
| | | GAAATTTGCCCTGAAGCACAACCTGCTGACCCATACCAAGATACACA |
| | | CGGGCGAGAAGCCCTTCCAGTGTCGAATCTGCATGCAGAACTTCAGT |
| | | GACCAGTCCAACCTGCGCGCCCACATCCGCACCCACACCGGCGAGAA |
| | | GCCTTTTGCCTGTGACATTTGTGGGAGGAAATTTGCCCGCAACTTCT |
| | | CCCTGACCATGCATACCAAGATACACACCGGAGAGCGCGGCTTCCAG |
| | | TGTCGAATCTGCATGCGTAACTTCAGTCTGCGCCACGACCTGGAGCG |
| | | CCACATCCGCACCCACACCGGCGAGAAGCCTTTTGCCTGTGACATTT |
| | | GTGGGAGGAAATTTGCCCACCGCTCCAACCTGAACAAGCATACCAAG |
| | | ATACACCTGCGGGGATCCCAGCTGGTGAAGAGCGAGCTGGAGGAGAA |
| | | GAAGTCCGAGCTGCGGCACAAGCTGAAGTACGTGCCCCACGAGTACA |
| | | TCGAGCTGATCGAGATCGCCAGGAACAGCACCCAGGACCGCATCCTG |
| | | GAGATGAAGGTGATGGAGTTCTTCATGAAGGTGTACGGCTACAGGGG |
| | | AAAGCACCTGGGCGGAAGCAGAAAGCCTGACGGCGCCATCTATACAG |
| | | TGGGCAGCCCCATCGATTACGGCGTGATCGTGGACACAAAGGCCTAC |
| | | AGCGGCGGCTACAATCTGAGCATCGGCCAGGCCGACGAGATGCAGAG |
| | | ATACGTGAAGGAGAACCAGACCCGGAATAAGCACATCAACCCCAACG |
| | | AGTGGTGGAAGGTGTACCCTAGCAGCGTGACCGAGTTCAAGTTCCTG |
| | | TTCGTGAGCGGCCACTTCAAGGGCAACTACAAGGCCCAGCTGACCAG |
| | | GCTGAACCGCAAAACCAACTGCAATGGCGCCGTGCTGAGCGTGGAGG |
| | | AGCTGCTGATCGGCGGCGAGATGATCAAAGCCGGCACCCTGACACTG |
| | | GAGGAGGTGCGGCGCAAGTTCAACAACGGCGAGATCAACTTC |
| 93 | Left ZFN-T2A- | GACTACAAGGACCACGACGGTGACTACAAAGACCACGATATAGACTA |
| | Right ZFN | TAAAGATGACGATGATAAGATGGCACCTAAAAAAAAGCGGAAAGTGG |
| | with N-terminal | GAATTCACGGCGTGCCCGCCGCCATGGCAGAGAGACCCTTTCAATGT |
| | modifications | AGAATCTGTATGCAAAATTTCTCTCAGAGTGGTAACCTTGCAAGACA |
| | (comprising | CATCAGAACTCATACAGGTGAGAAGCCGTTTGCATGTGACATTTGCG |
| | 3xFLAG, NLS, | GTAGGAAATTTGCCTTGAAACAGAATCTTTGTATGCACACAAAAATC |
| | ZFP-L, FokI, | CATACTGGTGAAAAGCCATTCCAATGCCGCATCTGTATGCAAAAATT |
| | T2A, 3xFLAG, | CGCGTGGCAGTCCAATTTGCAGAACCATACCAAGATTCACACGGGAG |
| | NLS, ZFP-R, | AAAAACCATTTCAGTGCCGCATCTGCATGCGCAACTTTTCTACATCA |
| | and FokI)(na) | GGAAACCTTACACGACATATTCGGACGCACACTGGAGAAAAACCATT |
| | ZFN-L | TGCTTGTGACATATGCGGCCGAAAATTTGCCAGACGCTCTCATCTCA |
| | Codon | CCTCACATACTAAGATTCATTTGCGCGGAAGTCAGCTGGTGAAGAGT |
| | diversified | GAATTGGAAGAAAAAAAGTCAGAGCTGAGACACAAACTGAAATATGT |
| | Version 2 | TCCACACGAGTAGATCGAGCTTATCGAGATAGCAAGAAACTCCACCC |
| | ZFN-R | AGGACAGAATTTTGGAAATGAAAGTTATGGAATTCTTTATGAAAGTG |
| | Not diversified | TATGGCTACAGGGGTAAACATCTGGGGGGATCAAGAAAGCCTGATGG |
| | | TGCAATTTACACAGTGGGCTCTCCTATCGACTACGGTGTGATCGTGG |
| | | ATACAAAGGCCTACTCTGGAGGATATAATTTGCCTATTGGACAAGCC |
| | | GATGAAATGGAAAGATATGTGGAGGAAAACCAGACTCGCGATAAGCA |
| | | CCTGAACCCAAATGAATGGTGGAAAGTGTACCCTTCATCTGTTACCG |
| | | AATTTAAATTTTTGTTCGTTTCCGGGCATTTCAAGGGGAACTACAAG |
| | | GCACAGCTGACGAGACTGAATCACATCACGAACTGCGACGGCGCTGT |
| | | ACTGTCCGTGGAAGAGCTTTTGATCGGGGGCGAAATGATTAAGGCCG |
| | | GCACACTGACGCTGGAGGAGGTGCGGCGAAAATTTAATAATGGCGAG |
| | | ATCAATTTTAGGAGTGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGACCATGACGGTGATTATAAAGATCATGACATCGATTACAAG |
| | | GATGACGATGACAAGATGGCCCCCAAGAAGAAGAGGAAGGTCGGCAT |
| | | TCATGGGGTACCCGCCGCTATGGCTGAGAGGCCCTTCCAGTGTCGAA |
| | | TCTGCATGCGTAACTTCAGTCAGTCCTCCGACCTGTCCCGCCACATC |
| | | CGCACCCACACCGGCGAGAAGCCTTTTGCCTGTGACATTTGTGGGAG |
| | | GAAATTTGCCCTGAAGCACAACCTGCTGACCCATACCAAGATACACA |
| | | CGGGCGAGAAGCCCTTCCAGTGTCGAATCTGCATGCAGAACTTCAGT |
| | | GACCAGTCCAACCTGCGCGCCCACATCCGCACCCACACCGGCGAGAA |
| | | GCCTTTTGCCTGTGACATTTGTGGGAGGAAATTTGCCCGCAACTTCT |
| | | CCCTGACCATGCATACCAAGATACACACCGGAGAGCGCGGCTTCCAG |
| | | TGTCGAATCTGCATGCGTAACTTCAGTCTGCGCCACGACCTGGAGCG |
| | | CCACATCCGCACCCACACCGGCGAGAAGCCTTTTGCCTGTGACATTT |
| | | GTGGGAGGAAATTTGCCCACCGCTCCAACCTGAACAAGCATACCAAG |
| | | ATACACCTGCGGGGATCCCAGCTGGTGAAGAGCGAGCTGGAGGAGAA |
| | | GAAGTCCGAGCTGCGGCACAAGCTGAAGTACGTGCCCCACGAGTACA |
| | | TCGAGCTGATCGAGATCGCCAGGAACAGCACCCAGGACCGCATCCTG |
| | | GAGATGAAGGTGATGGAGTTCTTCATGAAGGTGTACGGCTACAGGGG |
| | | AAAGCACCTGGGCGGAAGCAGAAAGCCTGACGGCGCCATCTATACAG |
| | | TGGGCAGCCCCATCGATTACGGCGTGATCGTGGACACAAAGGCCTAC |
| | | AGCGGCGGCTACAATCTGAGCATCGGCCAGGCCGACGAGATGCAGAG |
| | | ATACGTGAAGGAGAACCAGACCCGGAATAAGCACATCAACCCCAACG |
| | | AGTGGTGGAAGGTGTACCCTAGCAGCGTGACCGAGTTCAAGTTCCTG |
| | | TTCGTGAGCGGCCACTTCAAGGGCAACTACAAGGCCCAGCTGACCAG |
| | | GCTGAACCGCAAAACCAACTGCAATGGCGCCGTGCTGAGCGTGGAGG |
| | | AGCTGCTGATCGGCGGCGAGATGATCAAAGCCGGCACCCTGACACTG |
| | | GAGGAGGTGCGGCGCAAGTTCAACAACGGCGAGATCAACTTC |
| 94 | Left ZFN-T2A- | GACTATAAAGACCACGATGGCGACTAGAAAGACCACGACATCGATTA |
| | Right ZFN | CAAGGACGATGATGACAAAATGGCACCTAAGAAGAAGAGAAAAGTTG |
| | with N-terminal | GAATACATGGAGTCCCCGCAGCAATGGCCGAGAGACCTTTTCAGTGC |
| | modifications | AGGATTTGTATGCAAAACTTCTCTCAGTCCGGTAACCTGGCCCGGCA |
| | (comprising | CATACGAACACATACCGGCGAAAAACCCTTTGCTTGCGACATCTGCG |
| | 3xFLAG, NLS, | GAAGAAAGTTCGCTCTTAAACAGAACCTGTGCATGCATACAAAAATT |
| | ZFP-L, FokI, | CATACAGGTGAGAAGCCATTCCAATGCAGAATATGTATGCAGAAATT |
| | T2A, 3xFLAG, | CGCCTGGCAAAGCAACCTGCAAAACCACACTAAGATCCACACAGGGG |
| | NLS, ZFP-R, | AAAAGCCTTTTCAATGTAGAATCTGTATGAGAAACTTTAGTACATCC |
| | and FokI)(na) | GGAAATCTCACACGACATATCAGAACCCACACTGGAGAAAAACCTTT |
| | ZFN-L | TGCCTGCGACATCTGCGGAAGAAAATTCGCCCGAAGGTCCCACTTGA |
| | Codon | CTAGTCATACCAAAATCCACTTGCGAGGCTCACAGCTGGTTAAATCC |
| | diversified | GAACTTGAAGAAAAAAAAAGTGAACTGCGGCATAAACTGAAGTATGT |
| | Version 4 | CCCCCATGAATATATCGAACTGATAGAAATCGCCCGAAATAGCACCC |
| | ZFN-R | AAGATAGAATCCTCGAAATGAAGGTTATGGAATTTTTCATGAAGGTC |
| | Not diversified | TATGGATATAGGGGCAAGCACCTTGGCGGATCCCGGAAACCTGATGG |
| | | AGCTATCTAGACAGTGGGCTCACCAATAGACTATGGAGTTATCGTCG |
| | | ATACAAAAGCATACAGCGGAGGATACAATTTGCCAATAGGTCAAGCA |
| | | GATGAGATGGAAAGATACGTGGAGGAAAACCAAACAAGAGATAAGCA |
| | | TCTGAACCCCAACGAATGGTGGAAAGTGTACCCCAGTTCTGTAACCG |
| | | AATTTAAGTTCTTGTTCGTTTCAGGTCACTTCAAGGGTAATTACAAG |
| | | GCTCAACTGACTAGACTCAACCATATTACAAATTGCGATGGTGCTGT |
| | | GCTTTCCGTGGAAGAATTGCTGATTGGTGGAGAGATGATAAAAGCTG |
| | | GTACCCTCACCTTGGAAGAAGTGCGCAGAAAATTCAATAATGGCGAG |
| | | ATCAACTTCCGAAGTGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGACCATGACGGTGATTATAAAGATCATGACATCGATTACAAG |
| | | GATGACGATGACAAGATGGCCCCCAAGAAGAAGAGGAAGGTCGGCAT |
| | | TCATGGGGTACCCGCCGCTATGGCTGAGAGGCCCTTCCAGTGTCGAA |
| | | TCTGCATGCGTAACTTCAGTCAGTCCTCCGACCTGTCCCGCCACATC |
| | | CGCACCCACACCGGCGAGAAGCCTTTTGCCTGTGACATTTGTGGGAG |
| | | GAAATTTGCCCTGAAGCACAACCTGCTGACCCATACCAAGATACACA |
| | | CGGGCGAGAAGCCCTTCCAGTGTCGAATCTGCATGCAGAACTTCAGT |
| | | GACCAGTCCAACCTGCGCGCCCACATCCGCACCCACACCGGCGAGAA |
| | | GCCTTTTGCCTGTGACATTTGTGGGAGGAAATTTGCCCGCAACTTCT |
| | | CCCTGACCATGCATACCAAGATACACACCGGAGAGCGCGGCTTCCAG |
| | | TGTCGAATCTGCATGCGTAACTTCAGTCTGCGCCACGACCTGGAGCG |
| | | CCACATCCGCACCCACACCGGCGAGAAGCCTTTTGCCTGTGACATTT |
| | | GTGGGAGGAAATTTGCCCACCGCTCCAACCTGAACAAGCATACCAAG |
| | | ATACACCTGCGGGGATCCCAGCTGGTGAAGAGCGAGCTGGAGGAGAA |
| | | GAAGTCCGAGCTGCGGCACAAGCTGAAGTACGTGCCCCACGAGTACA |
| | | TCGAGCTGATCGAGATCGCCAGGAACAGCACCCAGGACCGCATCCTG |
| | | GAGATGAAGGTGATGGAGTTCTTCATGAAGGTGTACGGCTACAGGGG |
| | | AAAGCACCTGGGCGGAAGCAGAAAGCCTGACGGCGCCATCTATACAG |
| | | TGGGCAGCCCCATCGATTACGGCGTGATCGTGGACACAAAGGCCTAC |
| | | AGCGGCGGCTACAATCTGAGCATCGGCCAGGCCGACGAGATGCAGAG |
| | | ATACGTGAAGGAGAACCAGACCCGGAATAAGCACATCAACCCCAACG |
| | | AGTGGTGGAAGGTGTACCCTAGCAGCGTGACCGAGTTCAAGTTCCTG |
| | | TTCGTGAGCGGCCACTTCAAGGGCAACTACAAGGCCCAGCTGACCAG |
| | | GCTGAACCGCAAAACCAACTGCAATGGCGCCGTGCTGAGCGTGGAGG |
| | | AGCTGCTGATCGGCGGCGAGATGATCAAAGCCGGCACCCTGACACTG |
| | | GAGGAGGTGCGGCGCAAGTTCAACAACGGCGAGATCAACTTC |
| 95 | Left ZFN-T2A- | GATTATAAGGACCATGACGGAGACTATAAAGACCATGATATTGACTA |
| | Right ZFN | CAAAGACGACGATGATAAGATGGCCCCCAAGAAGAAACGAAAAGTAG |
| | with N-terminal | GAATCCATGGCGTGCCTGCAGCAATGGCAGAGAGACCATTTCAGTGC |
| | modifications | AGAATATGTATGCAAAACTTCTCCCAGAGCGGTAATCTGGCTAGGCA |
| | (comprising | TATTAGAACACACACCGGGGAAAAACCTTTCGCTTGCGATATATGTG |
| | 3xFLAG, NLS, | GTAGAAAGTTCGCCCTCAAACAGAATCTGTGCATGCACACTAAAATC |
| | ZFP-L, FokI, | CATACAGGAGAAAAGCCCTTTCAGTGTAGAATTTGTATGCAGAAATT |
| | T2A, 3xFLAG, | TGCTTGGCAGTCAAATTTGCAAAATCACACCAAAATACACACAGGAG |
| | NLS, ZFP-R, | AAAAACCATTTCAGTGTAGAATATGTATGAGAAATTTTTCCACTTCC |
| | and FokI)(na) | GGAAATCTGACCAGACATATACGGACACACACTGGGGAAAAGCCCTT |
| | ZFN-L | CGCTTGCGACATCTGCGGAAGAAAGTTCGCTAGACGGTCCCACTTGA |
| | Codon | CATCCCACACTAAGATACATCTTCGCGGTAGCCAACTGGTGAAAAGT |
| | diversified | GAACTGGAGGAAAAAAAATCTGAGCTGAGACATAAACTGAAATACGT |
| | Version 5 | ACCACATGAATACATAGAACTTATAGAAATAGCTAGGAACTCCACCC |
| | ZFN-R | AGGACAGAATACTTGAAATGAAGGTCATGGAGTTTTTTATGAAAGTT |
| | Not diversified | TACGGATACAGGGGCAAACACCTTGGAGGGTCTCGGAAGCCTGATGG |
| | | CGCAATTTATACCGTGGGTAGCCCTATAGATTATGGAGTGATTGTGG |
| | | ATACAAAGGCTTACAGTGGCGGCTATAATTTGCCTATCGGACAGGCC |
| | | GATGAGATGGAAAGATACGTTGAAGAAAACCAAACACGAGATAAGCA |
| | | TCTGAACCCCAATGAATGGTGGAAAGTGTATCCTTCAAGCGTTACCG |
| | | AGTTTAAGTTCCTCTTCGTTTCTGGGCATTTCAAGGGCAACTACAAA |
| | | GCTCAGCTTACAAGACTCAACCACATAACCAATTGTGATGGAGCAGT |
| | | CCTCAGCGTGGAAGAACTCCTTATTGGGGGTGAGATGATTAAAGCAG |
| | | GGACCCTTACTCTTGAAGAGGTTAGAAGAAAATTCAATAACGGAGAG |
| | | ATTAATTTTAGAAGTGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGACCATGACGGTGATTATAAAGATCATGACATCGATTACAAG |
| | | GATGACGATGACAAGATGGCCCCCAAGAAGAAGAGGAAGGTCGGCAT |
| | | TCATGGGGTACCCGCCGCTATGGCTGAGAGGCCCTTCCAGTGTCGAA |
| | | TCTGCATGCGTAACTTCAGTCAGTCCTCCGACCTGTCCCGCCACATC |
| | | CGCACCCACACCGGCGAGAAGCCTTTTGCCTGTGACATTTGTGGGAG |
| | | GAAATTTGCCCTGAAGCACAACCTGCTGACCCATACCAAGATACACA |
| | | CGGGCGAGAAGCCCTTCCAGTGTCGAATCTGCATGCAGAACTTCAGT |
| | | GACCAGTCCAACCTGCGCGCCCACATCCGCACCCACACCGGCGAGAA |
| | | GCCTTTTGCCTGTGACATTTGTGGGAGGAAATTTGCCCGCAACTTCT |
| | | CCCTGACCATGCATACCAAGATACACACCGGAGAGCGCGGCTTCCAG |
| | | TGTCGAATCTGCATGCGTAACTTCAGTCTGCGCCACGACCTGGAGCG |
| | | CCACATCCGCACCCACACCGGCGAGAAGCCTTTTGCCTGTGACATTT |
| | | GTGGGAGGAAATTTGCCCACCGCTCCAACCTGAACAAGCATACCAAG |
| | | ATACACCTGCGGGGATCCCAGCTGGTGAAGAGCGAGCTGGAGGAGAA |
| | | GAAGTCCGAGCTGCGGCACAAGCTGAAGTACGTGCCCCACGAGTACA |
| | | TCGAGCTGATCGAGATCGCCAGGAACAGCACCCAGGACCGCATCCTG |
| | | GAGATGAAGGTGATGGAGTTCTTCATGAAGGTGTACGGCTACAGGGG |
| | | AAAGCACCTGGGCGGAAGCAGAAAGCCTGACGGCGCCATCTATACAG |
| | | TGGGCAGCCCCATCGATTACGGCGTGATCGTGGACACAAAGGCCTAC |
| | | AGCGGCGGCTACAATCTGAGCATCGGCCAGGCCGACGAGATGCAGAG |
| | | ATACGTGAAGGAGAACCAGACCCGGAATAAGCACATCAACCCCAACG |
| | | AGTGGTGGAAGGTGTACCCTAGCAGCGTGACCGAGTTCAAGTTCCTG |
| | | TTCGTGAGCGGCCACTTCAAGGGCAACTACAAGGCCCAGCTGACCAG |
| | | GCTGAACCGCAAAACCAACTGCAATGGCGCCGTGCTGAGCGTGGAGG |
| | | AGCTGCTGATCGGCGGCGAGATGATCAAAGCCGGCACCCTGACACTG |
| | | GAGGAGGTGCGGCGCAAGTTCAACAACGGCGAGATCAACTTC |
| 96 | Left ZFN-T2A- | GACTATAAGGACCATGATGGAGACTATAAAGATCACGATATTGACTA |
| | Right ZFN | TAAAGATGATGATGATAAGATGGCACCTAAGAAGAAAAGAAAGGTCG |
| | with N-terminal | GCATTCATGGTGTGCCTGCAGCCATGGCCGAACGCCCATTTCAATGT |
| | modifications | AGAATTTGTATGCAGAATTTTTCACAATCAGGAAACCTGGCTAGACA |
| | (comprising | TATCAGAACACATACTGGAGAAAAGCCCTTTGCTTGTGATATCTGTG |
| | 3xFLAG, NLS, | GAAGGAAATTCGCCCTGAAACAAAACCTCTGTATGCACACAAAGATC |
| | ZFP-L, FokI, | CACACCGGCGAAAAGCCTTTCCAGTGTAGGATATGCATGCAAAAATT |
| | T2A, 3xFLAG, | CGCCTGGGAGTCCAATCTGCAGAACCATACCAAAATTCATACTGGTG |
| | NLS, ZFP-R, | AAAAGCCATTTCAGTGCAGAATATGTATGAGAAACTTTAGCACTTCA |
| | and FokI)(na) | GGAAATCTCACAAGACATATAAGAACACATACAGGGGAAAAACCTTT |
| | ZFN-L | TGCTTGCGATATCTGCGGCAGGAAATTCGCTCGGAGAAGTCATCTCA |
| | Codon | CAAGCCATACAAAAATCCACCTGCGAGGAAGCCAGCTGGTCAAGTCT |
| | diversified | GAACTGGAAGAAAAAAAAAGCGAACTGCGGCATAAACTCAAATACGT |
| | Version 6 | CCCACATGAATACATTGAGCTCATCGAAATTGCTAGAAACTCTACTC |
| | ZFN-R | AAGATAGGATATTGGAGATGAAGGTAATGGAATTCTTCATGAAGGTT |
| | Not diversified | TATGGATATAGAGGAAAACATCTTGGAGGCAGTAGGAAACCCGATGG |
| | | CGCTATCTACACCGTAGGGAGTCCAATCGACTACGGCGTGATTGTTG |
| | | ACACCAAAGCCTATTCTGGAGGGTATAATCTCCCAATTGGACAGGCA |
| | | GATGAGATGGAAAGATATGTAGAAGAAAATCAGACAAGAGATAAGCA |
| | | CCTTAACCCTAACGAGTGGTGGAAAGTGTACCCAAGCAGTGTTACTG |
| | | AATTTAAATTTCTTTTTGTATCAGGACACTTTAAAGGCAATTACAAA |
| | | GCACAACTGACCAGACTCAATCACATTACCAATTGCGACGGAGCCGT |
| | | ACTGAGCGTGGAGGAGTTGCTGATCGGAGGCGAAATGATTAAAGCTG |
| | | GCACTCTGACCCTGGAAGAAGTAAGAAGAAAGTTCAATAATGGAGAA |
| | | ATAAACTTTCGCTCCGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGACCATGACGGTGATTATAAAGATCATGACATCGATTACAAG |
| | | GATGACGATGACAAGATGGCCCCCAAGAAGAAGAGGAAGGTCGGCAT |
| | | TCATGGGGTACCCGCCGCTATGGCTGAGAGGCCCTTCCAGTGTCGAA |
| | | TCTGCATGCGTAACTTCAGTCAGTCCTCCGACCTGTCCCGCCACATC |
| | | CGCACCCACACCGGCGAGAAGCCTTTTGCCTGTGACATTTGTGGGAG |
| | | GAAATTTGCCCTGAAGCACAACCTGCTGACCCATACCAAGATACACA |
| | | CGGGCGAGAAGCCCTTCCAGTGTCGAATCTGCATGCAGAACTTCAGT |
| | | GACCAGTCCAACCTGCGCGCCCACATCCGCACCCACACCGGCGAGAA |
| | | GCCTTTTGCCTGTGACATTTGTGGGAGGAAATTTGCCCGCAACTTCT |
| | | CCCTGACCATGCATACCAAGATACACACCGGAGAGCGCGGCTTCCAG |
| | | TGTCGAATCTGCATGCGTAACTTCAGTCTGCGCCACGACCTGGAGCG |
| | | CCACATCCGCACCCACACCGGCGAGAAGCCTTTTGCCTGTGACATTT |
| | | GTGGGAGGAAATTTGCCCACCGCTCCAACCTGAACAAGCATACCAAG |
| | | ATACACCTGCGGGGATCCCAGCTGGTGAAGAGCGAGCTGGAGGAGAA |
| | | GAAGTCCGAGCTGCGGCACAAGCTGAAGTACGTGCCCCACGAGTACA |
| | | TCGAGCTGATCGAGATCGCCAGGAACAGCACCCAGGACCGCATCCTG |
| | | GAGATGAAGGTGATGGAGTTCTTCATGAAGGTGTACGGCTACAGGGG |
| | | AAAGCACCTGGGCGGAAGCAGAAAGCCTGACGGCGCCATCTATACAG |
| | | TGGGCAGCCCCATCGATTACGGCGTGATCGTGGACACAAAGGCCTAC |
| | | AGCGGCGGCTACAATCTGAGCATCGGCCAGGCCGACGAGATGCAGAG |
| | | ATACGTGAAGGAGAACCAGACCCGGAATAAGCACATCAACCCCAACG |
| | | AGTGGTGGAAGGTGTACCCTAGCAGCGTGACCGAGTTCAAGTTCCTG |
| | | TTCGTGAGCGGCCACTTCAAGGGCAACTACAAGGCCCAGCTGACCAG |
| | | GCTGAACCGCAAAACCAACTGCAATGGCGCCGTGCTGAGCGTGGAGG |
| | | AGCTGCTGATCGGCGGCGAGATGATCAAAGCCGGCACCCTGACACTG |
| | | GAGGAGGTGCGGCGCAAGTTCAACAACGGCGAGATCAACTTC |
| 97 | Left ZFN-T2A- | GACTACAAAGACCATGACGGTGATTATAAAGATCATGACATCGATTA |
| | Right ZFN | CAAGGATGACGATGACAAGATGGCCCCCAAGAAGAAGAGGAAGGTCG |
| | with N-terminal | GCATTCATGGGGTACCCGCCGCTATGGCTGAGAGGCCCTTCCAGTGT |
| | modifications | CGAATCTGCATGCAGAACTTCAGTCAGTCCGGCAACCTGGCCCGCCA |
| | (comprising | CATCCGCACCCACACCGGCGAGAAGCCTTTTGCCTGTGACATTTGTG |
| | 3xFLAG, NLS, | GGAGGAAATTTGCCCTGAAGCAGAACCTGTGTATGCATACCAAGATA |
| | ZFP-L, FokI, | CACACGGGCGAGAAGCCCTTCCAGTGTCGAATCTGCATGCAGAAGTT |
| | T2A, 3xFLAG, | TGCCTGGCAGTCCAACCTGCAGAACCATACCAAGATACACACGGGCG |
| | NLS, ZFP-R, | AGAAGCCCTTCCAGTGTCGAATCTGCATGCGTAACTTCAGTACCTCC |
| | and FokI)(na) | GGCAACCTGACCCGCCACATCCGCACCCACACCGGCGAGAAGCCTTT |
| | ZFN-L | TGCCTGTGACATTTGTGGGAGGAAATTTGCCCGCCGCTCCCACCTGA |
| | Not diversified | CCTCCCATACCAAGATACACCTGCGGGGATCCCAGCTGGTGAAGAGC |
| | ZFN-R | GAGCTGGAGGAGAAGAAGTCCGAGCTGCGGCACAAGCTGAAGTACGT |
| | Not diversified | GCCCCACGAGTAGATCGAGCTGATCGAGATCGCGAGGAACAGCACCC |
| | | AGGACCGCATCCTGGAGATGAAGGTGATGGAGTTCTTCATGAAGGTG |
| | | TACGGCTACAGGGGAAAGCACCTGGGCGGAAGCAGAAAGCCTGACGG |
| | | CGCCATCTATACAGTGGGCAGCCCCATCGATTACGGCGTGATCGTGG |
| | | ACACAAAGGCCTACAGCGGCGGCTACAATCTGCCTATCGGCCAGGCC |
| | | GACGAGATGGAGAGATACGTGGAGGAGAACCAGACCCGGGATAAGCA |
| | | CCTCAACCCCAACGAGTGGTGGAAGGTGTACCCTAGCAGCGTGACCG |
| | | AGTTCAAGTTCCTGTTCGTGAGCGGCCACTTCAAGGGCAACTACAAG |
| | | GCCCAGCTGACCAGGCTGAACCACATCACCAACTGCGACGGCGCCGT |
| | | GCTGAGCGTGGAGGAGCTGCTGATCGGCGGCGAGATGATCAAAGCCG |
| | | GCACCCTGACACTGGAGGAGGTGCGGCGCAAGTTCAACAACGGCGAG |
| | | ATCAACTTCAGATCTGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGACCATGACGGTGATTATAAAGATCATGACATCGATTACAAG |
| | | GATGACGATGACAAGATGGCCCCCAAGAAGAAGAGGAAGGTCGGCAT |
| | | TCATGGGGTACCCGCCGCTATGGCTGAGAGGCCCTTCCAGTGTCGAA |
| | | TCTGCATGCGTAACTTCAGTCAGTCCTCCGACCTGTCCCGCCACATC |
| | | CGCACCCACACCGGCGAGAAGCCTTTTGCCTGTGACATTTGTGGGAG |
| | | GAAATTTGCCCTGAAGCACAACCTGCTGACCCATACCAAGATACACA |
| | | CGGGCGAGAAGCCCTTCCAGTGTCGAATCTGCATGCAGAACTTCAGT |
| | | GACCAGTCCAACCTGCGCGCCCACATCCGCACCCACACCGGCGAGAA |
| | | GCCTTTTGCCTGTGACATTTGTGGGAGGAAATTTGCCCGCAACTTCT |
| | | CCCTGACCATGCATACCAAGATACACACCGGAGAGCGCGGCTTCCAG |
| | | TGTCGAATCTGCATGCGTAACTTCAGTCTGCGCCACGACCTGGAGCG |
| | | CCACATCCGCACCCACACCGGCGAGAAGCCTTTTGCCTGTGACATTT |
| | | GTGGGAGGAAATTTGCCCACCGCTCCAACCTGAACAAGCATACCAAG |
| | | ATACACCTGCGGGGATCCCAGCTGGTGAAGAGCGAGCTGGAGGAGAA |
| | | GAAGTCCGAGCTGCGGCACAAGCTGAAGTACGTGCCCCACGAGTACA |
| | | TCGAGCTGATCGAGATCGCCAGGAACAGCACCCAGGACCGCATCCTG |
| | | GAGATGAAGGTGATGGAGTTCTTCATGAAGGTGTACGGCTACAGGGG |
| | | AAAGCACCTGGGCGGAAGCAGAAAGCCTGACGGCGCCATCTATACAG |
| | | TGGGCAGCCCCATCGATTACGGCGTGATCGTGGACACAAAGGCCTAC |
| | | AGCGGCGGCTACAATCTGAGCATCGGCCAGGCCGACGAGATGCAGAG |
| | | ATACGTGAAGGAGAACCAGACCCGGAATAAGCACATCAACCCCAACG |
| | | AGTGGTGGAAGGTGTACCCTAGCAGCGTGACCGAGTTCAAGTTCCTG |
| | | TTCGTGAGCGGCCACTTCAAGGGCAACTACAAGGCCCAGCTGACCAG |
| | | GCTGAACCGCAAAACCAACTGCAATGGCGCCGTGCTGAGCGTGGAGG |
| | | AGCTGCTGATCGGCGGCGAGATGATCAAAGCCGGCACCCTGACACTG |
| | | GAGGAGGTGCGGCGCAAGTTCAACAACGGCGAGATCAACTTC |
| 98 | Left ZFN-T2A- | GACTACAAGGACCACGACGGTGACTACAAAGACCACGATATAGACTA |
| | Right ZFN | TAAAGATGACGATGATAAGATGGCACCTAAAAAAAAGCGGAAAGTGG |
| | with N-terminal | GAATTCACGGCGTGCCCGCCGCCATGGCAGAGAGACCCTTTCAATGT |
| | modifications | AGAATCTGTATGCAAAATTTCTCTCAGAGTGGTAACCTTGCAAGACA |
| | (comprising | CATCAGAACTCATACAGGTGAGAAGCCGTTTGCATGTGACATTTGCG |
| | 3xFLAG, NLS, | GTAGGAAATTTGCCTTGAAACAGAATCTTTGTATGCACACAAAAATC |
| | ZFP-L, FokI, | CATACTGGTGAAAAGCCATTCCAATGCCGCATCTGTATGCAAAAATT |
| | T2A, 3xFLAG, | CGCGTGGCAGTCCAATTTGCAGAACCATACCAAGATTCACACGGGAG |
| | NLS, ZFP-R, | AAAAACCATTTCAGTGCCGCATCTGCATGCGCAACTTTTCTACATCA |
| | and FokI)(na) | GGAAACCTTAGACGACATATTCGGACGCACACTGGAGAAAAACCATT |
| | ZFN-L | TGCTTGTGACATATGCGGCCGAAAATTTGCCAGACGCTCTCATCTCA |
| | Codon | CCTCACATACTAAGATTCATTTGCGCGGAAGTCAGCTGGTGAAGAGT |
| | diversified | GAATTGGAAGAAAAAAAGTCAGAGCTGAGACACAAACTGAAATATGT |
| | Version 2 | TCCACACGAGTAGATCGAGCTTATCGAGATAGCAAGAAACTCCACCC |
| | ZFN-R | AGGACAGAATTTTGGAAATGAAAGTTATGGAATTCTTTATGAAAGTG |
| | Codon | TATGGCTACAGGGGTAAACATCTGGGGGGATCAAGAAAGCCTGATGG |
| | diversified | TGCAATTTACACAGTGGGCTCTCCTATCGACTACGGTGTGATCGTGG |
| | Version 4 | ATACAAAGGCCTACTCTGGAGGATATAATTTGCCTATTGGACAAGCC |
| | | GATGAAATGGAAAGATATGTGGAGGAAAACCAGACTCGCGATAAGCA |
| | | CCTGAACCCAAATGAATGGTGGAAAGTGTACCCTTCATCTGTTACCG |
| | | AATTTAAATTTTTGTTCGTTTCCGGGCATTTCAAGGGGAACTACAAG |
| | | GCACAGCTGACGAGACTGAATCACATCACGAACTGCGACGGCGCTGT |
| | | ACTGTCCGTGGAAGAGCTTTTGATCGGGGGCGAAATGATTAAGGCCG |
| | | GCACACTGACGCTGGAGGAGGTGCGGCGAAAATTTAATAATGGCGAG |
| | | ATCAATTTTAGGAGTGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGATCATGATGGCGACTACAAAGATCATGATATAGATTACAAA |
| | | GACGATGACGACAAAATGGCTCCAAAAAAAAAACGCAAGGTTGGAAT |
| | | ACACGGTGTACCTGCCGCTATGGCTGAAAGACCTTTCCAGTGTAGGA |
| | | TTTGCATGAGAAATTTTTCCCAATCATCCGACCTTTCAAGGCATATT |
| | | AGGACACACACCGGGGAAAAGCCATTTGCTTGTGATATCTGCGGGCG |
| | | CAAATTTGCTCTTAAGCACAATCTTCTTACCCACACCAAAATTCATA |
| | | CAGGAGAAAAACCTTTTCAATGTAGAATCTGCATGCAAAACTTTTCC |
| | | GATCAGTCAAATCTTAGAGCTCATATCAGAACCCATACCGGGGAGAA |
| | | ACCCTTTGCCTGCGACATATGCGGAAGAAAATTTGCTAGGAACTTTA |
| | | GTCTGACCATGCATACCAAAATTCATACCGGCGAACGCGGTTTCCAG |
| | | TGCAGGATTTGTATGAGAAATTTCTCACTGCGGCATGATCTTGAAAG |
| | | ACACATACGAACTCATACCGGAGAAAAGCCATTCGCTTGCGATATTT |
| | | GTGGTAGAAAATTTGCCCACAGGTCTAACCTTAATAAGCACACCAAG |
| | | ATTCATCTCAGAGGATCTCAGCTGGTCAAATCAGAACTTGAAGAGAA |
| | | AAAAAGCGAACTGAGACATAAACTGAAGTACGTGCCTCATGAATACA |
| | | TAGAGCTCATTGAAATAGCTAGGAATAGTACACAGGACAGGATACTT |
| | | GAAATGAAGGTAATGGAATTTTTCATGAAGGTTTATGGATACCGGGG |
| | | GAAACATCTCGGGGGCAGCAGAAAACCAGACGGAGCAATTTATACTG |
| | | TCGGGAGTCCTATAGATTATGGCGTTATCGTCGATACAAAGGCCTAT |
| | | TCCGGTGGGTACAACCTCTCAATTGGTCAGGCTGATGAGATGCAAAG |
| | | ATACGTCAAAGAAAACCAAACCAGAAATAAACATATAAATCCCAATG |
| | | AATGGTGGAAAGTATACCCAAGTTCCGTGACTGAATTCAAGTTCCTT |
| | | TTCGTGTCTGGCCACTTTAAAGGAAATTATAAAGCACAATTGACTAG |
| | | ACTGAATAGAAAAACAAACTGTAACGGCGCAGTGCTGTCAGTGGAAG |
| | | AACTGCTCATAGGTGGAGAGATGATCAAGGCCGGGACACTTACTCTT |
| | | GAGGAAGTTAGAAGGAAGTTCAACAACGGCGAAATCAACTTT |
| 99 | Right ZFN- | GACTACAAAGATCATGATGGCGACTACAAAGATCATGATATAGATTA |
| | T2A-Left ZFN | CAAAGACGATGACGACAAAATGGCTCCAAAAAAAAAACGCAAGGTTG |
| | with N-terminal | GAATACACGGTGTACCTGCCGCTATGGCTGAAAGACCTTTCCAGTGT |
| | modifications | AGGATTTGCATGAGAAATTTTTCCCAATCATCCGACCTTTCAAGGCA |
| | (comprising | TATTAGGACACACACCGGGGAAAAGCCATTTGCTTGTGATATCTGCG |
| | 3xFLAG, NLS, | GGCGCAAATTTGCTCTTAAGCACAATCTTCTTACCCACACCAAAATT |
| | ZFP-R, FokI, | CATACAGGAGAAAAACCTTTTCAATGTAGAATCTGCATGCAAAACTT |
| | T2A, 3xFLAG, | TTCCGATCAGTCAAATCTTAGAGCTCATATCAGAACCCATACCGGGG |
| | NLS, ZFP-L, | AGAAACCCTTTGCCTGCGACATATGCGGAAGAAAATTTGCTAGGAAC |
| | and FokI)(na) | TTTAGTCTGACCATGCATACCAAAATTCATACCGGCGAACGCGGTTT |
| | ZFN-R | CCAGTGCAGGATTTGTATGAGAAATTTCTCACTGCGGCATGATCTTG |
| | Codon | AAAGACACATACGAACTCATACCGGAGAAAAGCCATTCGCTTGCGAT |
| | diversified | ATTTGTGGTAGAAAATTTGCCCACAGGTCTAACCTTAATAAGCACAC |
| | Version 4 | CAAGATTCATCTCAGAGGATCTCAGCTGGTCAAATCAGAACTTGAAG |
| | ZFN-L | AGAAAAAAAGCGAACTGAGACATAAACTGAAGTACGTGCCTCATGAA |
| | Codon | TACATAGAGCTCATTGAAATAGCTAGGAATAGTACACAGGACAGGAT |
| | diversified | ACTTGAAATGAAGGTAATGGAATTTTTCATGAAGGTTTATGGATACC |
| | Version 2 | GGGGGAAACATCTCGGGGGCAGCAGAAAACCAGACGGAGCAATTTAT |
| | | ACTGTCGGGAGTCCTATAGATTATGGCGTTATCGTCGATACAAAGGC |
| | | CTATTCCGGTGGGTACAACCTCTCAATTGGTCAGGCTGATGAGATGC |
| | | AAAGATACGTCAAAGAAAACCAAACCAGAAATAAACATATAAATCCC |
| | | AATGAATGGTGGAAAGTATACCCAAGTTCCGTGACTGAATTCAAGTT |
| | | CCTTTTCGTGTCTGGCCACTTTAAAGGAAATTATAAAGCACAATTGA |
| | | CTAGACTGAATAGAAAAACAAACTGTAACGGCGCAGTGCTGTCAGTG |
| | | GAAGAACTGCTCATAGGTGGAGAGATGATCAAGGCCGGGACACTTAC |
| | | TCTTGAGGAAGTTAGAAGGAAGTTCAACAACGGCGAAATCAACTTTG |
| | | GCAGCGGAGAGGGCAGAGGAAGCCTGCTCACCTGCGGTGACGTGGAG |
| | | GAAAACCCTGGCCCTACGCGTGCCATGGACTACAAGGACCACGACGG |
| | | TGACTACAAAGACCACGATATAGACTATAAAGATGACGATGATAAGA |
| | | TGGCACCTAAAAAAAAGCGGAAAGTGGGAATTCACGGCGTGCCCGCC |
| | | GCCATGGCAGAGAGACCCTTTCAATGTAGAATCTGTATGCAAAATTT |
| | | CTCTCAGAGTGGTAACCTTGCAAGACACATCAGAACTCATACAGGTG |
| | | AGAAGCCGTTTGCATGTGACATTTGCGGTAGGAAATTTGCCTTGAAA |
| | | CAGAATCTTTGTATGCACACAAAAATCCATACTGGTGAAAAGCCATT |
| | | CCAATGCCGCATCTGTATGCAAAAATTCGCGTGGCAGTCCAATTTGC |
| | | AGAACCATACCAAGATTCACACGGGAGAAAAACCATTTCAGTGCCGC |
| | | ATCTGCATGCGCAACTTTTCTACATCAGGAAACCTTACACGACATAT |
| | | TCGGACGCACACTGGAGAAAAACCATTTGCTTGTGACATATGCGGCC |
| | | GAAAATTTGCCAGACGCTCTCATCTCACCTCACATACTAAGATTCAT |
| | | TTGCGCGGAAGTCAGCTGGTGAAGAGTGAATTGGAAGAAAAAAAGTC |
| | | AGAGCTGAGACACAAACTGAAATATGTTCCACACGAGTACATCGAGC |
| | | TTATCGAGATAGCAAGAAACTCCACCCAGGACAGAATTTTGGAAATG |
| | | AAAGTTATGGAATTCTTTATGAAAGTGTATGGCTACAGGGGTAAACA |
| | | TCTGGGGGGATCAAGAAAGCCTGATGGTGCAATTTACACAGTGGGCT |
| | | CTCCTATCGACTACGGTGTGATCGTGGATACAAAGGCCTACTCTGGA |
| | | GGATATAATTTGCCTATTGGACAAGCCGATGAAATGGAAAGATATGT |
| | | GGAGGAAAACCAGACTCGCGATAAGCACCTGAACCCAAATGAATGGT |
| | | GGAAAGTGTACCCTTCATCTGTTACCGAATTTAAATTTTTGTTCGTT |
| | | TCCGGGCATTTCAAGGGGAACTACAAGGCACAGCTGACGAGACTGAA |
| | | TCACATCACGAACTGCGACGGCGCTGTACTGTCCGTGGAAGAGCTTT |
| | | TGATCGGGGGCGAAATGATTAAGGCCGGCACACTGACGCTGGAGGAG |
| | | GTGCGGCGAAAATTTAATAATGGCGAGATCAATTTTAGGAGT |
| 100 | Right ZFN- | GTACCTGCTGCTATGGCTGAAAGACCTTTTCAATGTCGAATCTGCAT |
| | T2A-Left ZFN | GAGGAATTTTAGTCAGTCATCCGACCTGAGCAGACACATTCGAACCC |
| | (na) | ATACTGGTGAAAAGCCATTTGCTTGCGATATATGTGGGAGAAAATTT |
| | ZFN-R | GCGTTGAAACACAATCTGCTGACCCATACCAAGATTCATACCGGAGA |
| | Codon | AAAACCATTCCAATGCCGCATTTGTATGCAGAACTTTAGTGACCAGT |
| | diversified | CAAATCTCCGCGCTCACATTCGAACCCACACTGGCGAAAAACCCTTT |
| | Version 1 | GCTTGTGACATTTGCGGTCGGAAGTTTGCCCGAAATTTTTCTCTGAC |
| | ZFN-L | AATGCACACAAAAATCCACACCGGGGAACGCGGCTTTCAATGTAGGA |
| | Not diversified | TCTGTATGAGAAATTTTAGCCTTAGACATGATTTGGAACGACATATC |
| | | AGGACCCATACAGGCGAGAAACCATTTGCGTGCGATATTTGTGGCAG |
| | | GAAATTCGCACATAGAAGTAATCTGAACAAGCATACAAAAATTCATC |
| | | TCAGAGGAAGTCAGCTGGTCAAAAGTGAACTGGAGGAAAAAAAGAGC |
| | | GAACTGAGACACAAACTGAAGTACGTGCCACACGAATATATTGAGCT |
| | | GATTGAGATCGCGAGGAACTCAACACAGGACCGCATTCTGGAGATGA |
| | | AAGTGATGGAGTTTTTCATGAAAGTATATGGATATAGAGGAAAACAC |
| | | CTTGGGGGTAGCCGAAAGCCGGACGGGGCGATCTACACTGTGGGGTC |
| | | ACCAATTGATTATGGCGTAATTGTCGATACCAAAGCCTACAGTGGGG |
| | | GGTACAATCTGAGTATAGGACAGGCTGATGAAATGCAACGATACGTT |
| | | AAGGAGAATCAGACTAGGAATAAACATATCAATCCAAATGAATGGTG |
| | | GAAAGTCTATCCCAGCAGCGTGACAGAATTTAAATTTTTGTTTGTCA |
| | | GTGGACACTTCAAGGGAAATTATAAGGCCCAGCTGACTAGACTGAAT |
| | | AGGAAAACCAATTGTAATGGCGCAGTGCTTTCAGTGGAGGAACTGCT |
| | | CATTGGAGGTGAGATGATCAAGGCTGGAACCCTGACGCTGGAGGAGG |
| | | TGCGGAGGAAGTTTAACAATGGAGAAATTAACTTTGGCAGCGGAGAG |
| | | GGCAGAGGAAGCCTGCTCACCTGCGGTGACGTGGAGGAAAACCCTGG |
| | | CCCTGCCGCTATGGCTGAGAGGCCCTTCCAGTGTCGAATCTGCATGC |
| | | AGAACTTCAGTCAGTCCGGCAACCTGGCCCGCCACATCCGCACCCAC |
| | | ACCGGCGAGAAGCCTTTTGCCTGTGACATTTGTGGGAGGAAATTTGC |
| | | CCTGAAGCAGAACCTGTGTATGCATACCAAGATACACACGGGCGAGA |
| | | AGCCCTTCCAGTGTCGAATCTGCATGCAGAAGTTTGCCTGGCAGTCC |
| | | AACCTGCAGAACCATACCAAGATACACACGGGCGAGAAGCCCTTCCA |
| | | GTGTCGAATCTGCATGCGTAACTTCAGTACCTCCGGCAACCTGACCC |
| | | GCCACATCCGCACCCACACCGGCGAGAAGCCTTTTGCCTGTGACATT |
| | | TGTGGGAGGAAATTTGCCCGCCGCTCCCACCTGACCTCCCATACCAA |
| | | GATACACCTGCGGGGATCCCAGCTGGTGAAGAGCGAGCTGGAGGAGA |
| | | AGAAGTCCGAGCTGCGGCACAAGCTGAAGTACGTGCCCCACGAGTAC |
| | | ATCGAGCTGATCGAGATCGCCAGGAACAGCACCCAGGACCGCATCCT |
| | | GGAGATGAAGGTGATGGAGTTCTTCATGAAGGTGTACGGCTACAGGG |
| | | GAAAGCACCTGGGCGGAAGCAGAAAGCCTGACGGCGCCATCTATACA |
| | | GTGGGCAGCCCCATCGATTACGGCGTGATCGTGGACACAAAGGCCTA |
| | | CAGCGGCGGCTACAATCTGCCTATCGGCCAGGCCGACGAGATGGAGA |
| | | GATACGTGGAGGAGAACCAGACCCGGGATAAGCACCTCAACCCCAAC |
| | | GAGTGGTGGAAGGTGTACCCTAGCAGCGTGACCGAGTTCAAGTTCCT |
| | | GTTCGTGAGCGGCCACTTCAAGGGCAACTACAAGGCCCAGCTGACCA |
| | | GGCTGAACCACATCACCAACTGCGACGGCGCCGTGCTGAGCGTGGAG |
| | | GAGCTGCTGATCGGCGGCGAGATGATCAAAGCCGGCACCCTGACACT |
| | | GGAGGAGGTGCGGCGCAAGTTCAACAACGGCGAGATCAACTTCAGAT |
| | | CT |
| 101 | Right ZFN- | GTCCCAGCTGCCATGGCCGAGAGACCATTTCAATGTCGGATTTGCAT |
| | T2A-Left ZFN | GCGCAATTTTTCCCAGTCCTCTGACCTTAGCCGGCATATTCGGACAC |
| | (na) | ACACAGGTGAAAAACCCTTCGCATGCGACATTTGCGGAAGAAAATTC |
| | ZFN-R | GCTCTGAAACACAACCTGCTTACCCATACAAAGATCCACACCGGCGA |
| | Codon | GAAACCGTTTCAATGCCGAATCTGTATGCAAAATTTTAGTGATCAAA |
| | diversified | GTAATCTGAGAGCACATATTAGGACTCACACGGGCGAGAAGCCATTT |
| | Version 2 | GCGTGTGATATCTGCGGCCGAAAATTCGCCCGGAATTTCTCTCTGAC |
| | ZFN-L | AATGCACACCAAAATCCACACTGGGGAACGAGGCTTTCAATGTAGAA |
| | Not diversified | TATGTATGCGGAATTTCAGTCTGAGGCACGACCTGGAGCGGCACATC |
| | | AGAACTCACACCGGAGAAAAACCATTCGCTTGTGATATTTGCGGGAG |
| | | GAAGTTCGCCCATAGGAGCAATCTCAATAAACACACCAAAATACATC |
| | | TTCGGGGTTCTCAACTGGTGAAATCCGAACTGGAAGAAAAGAAATCA |
| | | GAATTGCGGCATAAACTGAAGTATGTGCCCCATGAGTACATAGAACT |
| | | GATCGAGATCGCAAGGAACTCTACCCAGGACAGAATACTTGAAATGA |
| | | AGGTCATGGAATTTTTTATGAAAGTGTACGGCTACAGAGGAAAACAT |
| | | TTGGGAGGCAGTCGAAAACCAGATGGCGCAATCTATACAGTCGGGTC |
| | | CCCCATAGATTACGGAGTGATTGTCGACACAAAAGCCTATTCCGGAG |
| | | GATATAACCTTAGTATCGGCCAGGCCGACGAGATGCAACGCTATGTG |
| | | AAAGAAAACCAAACAAGAAATAAACATATCAATCCAAACGAGTGGTG |
| | | GAAGGTATATCCAAGCAGTGTCACAGAATTCAAATTCCTCTTCGTGA |
| | | GTGGGCACTTTAAAGGCAACTACAAAGCTCAATTGACCAGGCTCAAT |
| | | CGGAAAACTAATTGCAATGGCGCAGTCCTTAGCGTCGAAGAATTGCT |
| | | GATTGGCGGGGAAATGATTAAAGCAGGAACTTTGACCTTGGAGGAAG |
| | | TACGGAGAAAGTTTAACAACGGCGAGATTAATTTTGGCAGCGGAGAG |
| | | GGCAGAGGAAGCCTGCTCACCTGCGGTGACGTGGAGGAAAACCCTGG |
| | | CCCTGCCGCTATGGCTGAGAGGCCCTTCCAGTGTCGAATCTGCATGC |
| | | AGAACTTCAGTCAGTCCGGCAACCTGGCCCGCCACATCCGCACCCAC |
| | | ACCGGCGAGAAGCCTTTTGCCTGTGACATTTGTGGGAGGAAATTTGC |
| | | CCTGAAGCAGAACCTGTGTATGCATACCAAGATACACACGGGCGAGA |
| | | AGCCCTTCCAGTGTCGAATCTGCATGCAGAAGTTTGCCTGGCAGTCC |
| | | AACCTGCAGAACCATACCAAGATACACACGGGCGAGAAGCCCTTCCA |
| | | GTGTCGAATCTGCATGCGTAACTTCAGTACCTCCGGCAACCTGACCC |
| | | GCCACATCCGCACCCACACCGGCGAGAAGCCTTTTGCCTGTGACATT |
| | | TGTGGGAGGAAATTTGCCCGCCGCTCCCACCTGACCTCCCATACCAA |
| | | GATACACCTGCGGGGATCCCAGCTGGTGAAGAGCGAGCTGGAGGAGA |
| | | AGAAGTCCGAGCTGCGGCACAAGCTGAAGTACGTGCCCCACGAGTAC |
| | | ATCGAGCTGATCGAGATCGCCAGGAACAGCACCCAGGACCGCATCCT |
| | | GGAGATGAAGGTGATGGAGTTCTTCATGAAGGTGTACGGCTACAGGG |
| | | GAAAGCACCTGGGCGGAAGCAGAAAGCCTGACGGCGCCATCTATACA |
| | | GTGGGCAGCCCCATCGATTACGGCGTGATCGTGGACACAAAGGCCTA |
| | | CAGCGGCGGCTACAATCTGCCTATCGGCCAGGCCGACGAGATGGAGA |
| | | GATACGTGGAGGAGAACCAGACCCGGGATAAGCACCTCAACCCCAAC |
| | | GAGTGGTGGAAGGTGTACCCTAGCAGCGTGACCGAGTTCAAGTTCCT |
| | | GTTCGTGAGCGGCCACTTCAAGGGCAACTACAAGGCCCAGCTGACCA |
| | | GGCTGAACCACATCACCAACTGCGACGGCGCCGTGCTGAGCGTGGAG |
| | | GAGCTGCTGATCGGCGGCGAGATGATCAAAGCCGGCACCCTGACACT |
| | | GGAGGAGGTGCGGCGCAAGTTCAACAACGGCGAGATCAACTTCAGAT |
| | | CT |
| 102 | Right ZFN- | GTCCCTGCCGCCATGGCCGAGCGCCCCTTCCAATGCCGCATATGCAT |
| | T2A-Left ZFN | GAGAAATTTCAGCCAAAGTAGCGACCTGTCACGACACATTAGAACTC |
| | (na) | ATACGGGGGAGAAGCCATTTGCTTGCGATATTTGTGGCAGAAAATTC |
| | ZFN-R | GCACTCAAACACAACCTGCTCACACACACCAAGATACACACGGGAGA |
| | Codon | GAAGCCCTTCCAATGTAGAATATGTATGCAAAATTTCAGCGACCAAA |
| | diversified | GTAATTTGAGAGCGCATATTCGAACTCACACCGGCGAAAAACCATTT |
| | Version 3 | GCCTGCGATATTTGTGGGAGGAAATTTGCCAGGAATTTTTCACTCAC |
| | ZFN-L | CATGCACACTAAGATCCACACTGGCGAGCGCGGCTTCCAATGCAGAA |
| | Not diversified | TCTGTATGCGAAACTTCAGTCTGCGGCATGACCTGGAAAGACATATA |
| | | AGAACCCACACCGGAGAAAAACCCTTTGCCTGCGACATATGTGGTAG |
| | | AAAATTCGCACATCGGAGTAACCTTAACAAACATACAAAGATCCACT |
| | | TGAGAGGCAGTCAGCTGGTGAAATCTGAGCTGGAAGAGAAGAAATCT |
| | | GAACTGCGACATAAATTGAAGTACGTCCCACACGAGTAGATCGAGTT |
| | | GATCGAAATTGCCCGGAATAGCACCCAGGATAGAATATTGGAAATGA |
| | | AAGTAATGGAGTTTTTTATGAAGGTTTATGGTTACAGAGGCAAGCAC |
| | | CTTGGAGGAAGCAGGAAACCAGATGGGGCGATTTACACCGTTGGGAG |
| | | TCCCATCGATTACGGAGTCATCGTGGACACAAAGGCCTATTCCGGAG |
| | | GCTACAACCTCAGTATCGGGCAAGCCGATGAGATGCAGAGATATGTT |
| | | AAAGAAAATCAGACGCGAAACAAGCACATTAACCCAAACGAATGGTG |
| | | GAAAGTTTACCCTAGCTCAGTGACAGAATTTAAGTTTCTGTTTGTCA |
| | | GCGGCCACTTCAAGGGGAATTATAAAGCACAACTGACCCGCCTGAAC |
| | | CGAAAAACCAACTGTAACGGTGCTGTGCTGAGTGTCGAAGAGTTGCT |
| | | TATCGGAGGAGAGATGATAAAGGCCGGCACACTGACGCTTGAAGAGG |
| | | TACGGCGAAAATTCAATAACGGAGAGATTAATTTTGGCAGCGGAGAG |
| | | GGCAGAGGAAGCCTGCTCACCTGCGGTGACGTGGAGGAAAACCCTGG |
| | | CCCT |
| | | GCCGCTATGGCTGAGAGGCCCTTCCAGTGTCGAATCTGCATGCAGAA |
| | | CTTCAGTCAGTCCGGCAACCTGGCCCGCCACATCCGCACCCACACCG |
| | | GCGAGAAGCCTTTTGCCTGTGACATTTGTGGGAGGAAATTTGCCCTG |
| | | AAGCAGAACCTGTGTATGCATACCAAGATACACACGGGCGAGAAGCC |
| | | CTTCCAGTGTCGAATCTGCATGCAGAAGTTTGCCTGGCAGTCCAACC |
| | | TGCAGAACCATACCAAGATACACACGGGCGAGAAGCCCTTCCAGTGT |
| | | CGAATCTGCATGCGTAACTTCAGTACCTCCGGCAACCTGACCCGCCA |
| | | CATCCGCACCCACACCGGCGAGAAGCCTTTTGCCTGTGACATTTGTG |
| | | GGAGGAAATTTGCCCGCCGCTCCCACCTGACCTCCCATACCAAGATA |
| | | CACCTGCGGGGATCCCAGCTGGTGAAGAGCGAGCTGGAGGAGAAGAA |
| | | GTCCGAGCTGCGGCACAAGCTGAAGTACGTGCCCCACGAGTACATCG |
| | | AGCTGATCGAGATCGCCAGGAACAGCACCCAGGACCGCATCCTGGAG |
| | | ATGAAGGTGATGGAGTTCTTCATGAAGGTGTACGGCTACAGGGGAAA |
| | | GCACCTGGGCGGAAGCAGAAAGCCTGACGGCGCCATCTATACAGTGG |
| | | GCAGCCCCATCGATTACGGCGTGATCGTGGACACAAAGGCCTACAGC |
| | | GGCGGCTACAATCTGCCTATCGGCCAGGCCGACGAGATGGAGAGATA |
| | | CGTGGAGGAGAACCAGACCCGGGATAAGCACCTCAACCCCAACGAGT |
| | | GGTGGAAGGTGTACCCTAGCAGCGTGACCGAGTTCAAGTTCCTGTTC |
| | | GTGAGCGGCCACTTCAAGGGCAACTACAAGGCCCAGCTGACCAGGCT |
| | | GAACCACATCACCAACTGCGACGGCGCCGTGCTGAGCGTGGAGGAGC |
| | | TGCTGATCGGCGGCGAGATGATCAAAGCCGGCACCCTGACACTGGAG |
| | | GAGGTGCGGCGCAAGTTCAACAACGGCGAGATCAACTTCAGATCT |
| 103 | Right ZFN- | GTACCTGCCGCTATGGCTGAAAGACCTTTCCAGTGTAGGATTTGCAT |
| | T2A-Left ZFN | GAGAAATTTTTCCCAATCATCCGAGCTTTCAAGGCATATTAGGAGAC |
| | (na) | ACACCGGGGAAAAGCCATTTGCTTGTGATATCTGCGGGCGCAAATTT |
| | ZFN-R | GCTCTTAAGCACAATCTTCTTACCCACACCAAAATTCATACAGGAGA |
| | Codon | AAAACCTTTTCAATGTAGAATCTGCATGCAAAACTTTTCCGATCAGT |
| | diversified | CAAATCTTAGAGCTCATATCAGAACCCATACCGGGGAGAAACCCTTT |
| | Version 4 | GCCTGCGACATATGCGGAAGAAAATTTGCTAGGAACTTTAGTCTGAC |
| | ZFN-L | CATGCATACCAAAATTCATACCGGCGAACGCGGTTTCCAGTGCAGGA |
| | Not diversified | TTTGTATGAGAAATTTCTCACTGCGGCATGATCTTGAAAGACACATA |
| | | CGAACTCATACCGGAGAAAAGCCATTCGCTTGCGATATTTGTGGTAG |
| | | AAAATTTGCCCACAGGTCTAACCTTAATAAGCACACCAAGATTCATC |
| | | TCAGAGGATCTCAGCTGGTCAAATCAGAACTTGAAGAGAAAAAAAGC |
| | | GAACTGAGACATAAACTGAAGTACGTGCCTCATGAATACATAGAGCT |
| | | CATTGAAATAGCTAGGAATAGTACACAGGACAGGATACTTGAAATGA |
| | | AGGTAATGGAATTTTTCATGAAGGTTTATGGATACCGGGGGAAACAT |
| | | CTCGGGGGCAGCAGAAAACCAGACGGAGCAATTTATACTGTCGGGAG |
| | | TCCTATAGATTATGGCGTTATCGTCGATACAAAGGCCTATTCCGGTG |
| | | GGTACAACCTCTCAATTGGTCAGGCTGATGAGATGCAAAGATACGTC |
| | | AAAGAAAACCAAACGAGAAATAAACATATAAATCCCAATGAATGGTG |
| | | GAAAGTATACCCAAGTTCCGTGACTGAATTCAAGTTCCTTTTCGTGT |
| | | CTGGCCACTTTAAAGGAAATTATAAAGCACAATTGACTAGACTGAAT |
| | | AGAAAAACAAACTGTAACGGCGCAGTGCTGTCAGTGGAAGAACTGCT |
| | | CATAGGTGGAGAGATGATCAAGGCCGGGACACTTACTCTTGAGGAAG |
| | | TTAGAAGGAAGTTCAACAACGGCGAAATCAACTTTGGCAGCGGAGAG |
| | | GGCAGAGGAAGCCTGCTCACCTGCGGTGACGTGGAGGAAAACCCTGG |
| | | CCCTGCCGCTATGGCTGAGAGGCCCTTCCAGTGTCGAATCTGCATGC |
| | | AGAACTTCAGTCAGTCCGGCAACCTGGCCCGCCACATCCGCACCCAC |
| | | ACCGGCGAGAAGCCTTTTGCCTGTGACATTTGTGGGAGGAAATTTGC |
| | | CCTGAAGCAGAACCTGTGTATGCATACCAAGATACACACGGGCGAGA |
| | | AGCCCTTCCAGTGTCGAATCTGCATGCAGAAGTTTGCCTGGCAGTCC |
| | | AACCTGCAGAACCATACCAAGATACACACGGGCGAGAAGCCCTTCCA |
| | | GTGTCGAATCTGCATGCGTAACTTCAGTACCTCCGGCAACCTGACCC |
| | | GCCACATCCGCACCCACACCGGCGAGAAGCCTTTTGCCTGTGACATT |
| | | TGTGGGAGGAAATTTGCCCGCCGCTCCCACCTGACCTCCCATACCAA |
| | | GATACACCTGCGGGGATCCCAGCTGGTGAAGAGCGAGCTGGAGGAGA |
| | | AGAAGTCCGAGCTGCGGCACAAGCTGAAGTACGTGCCCCACGAGTAC |
| | | ATCGAGCTGATCGAGATCGCCAGGAACAGCACCCAGGACCGCATCCT |
| | | GGAGATGAAGGTGATGGAGTTCTTCATGAAGGTGTACGGCTACAGGG |
| | | GAAAGCACCTGGGCGGAAGCAGAAAGCCTGACGGCGCCATCTATACA |
| | | GTGGGCAGCCCCATCGATTACGGCGTGATCGTGGACACAAAGGCCTA |
| | | CAGCGGCGGCTACAATCTGCCTATCGGCCAGGCCGACGAGATGGAGA |
| | | GATACGTGGAGGAGAACCAGACCCGGGATAAGCACCTCAACCCCAAC |
| | | GAGTGGTGGAAGGTGTACCCTAGCAGCGTGACCGAGTTCAAGTTCCT |
| | | GTTCGTGAGCGGCCACTTCAAGGGCAACTACAAGGCCCAGCTGACCA |
| | | GGCTGAACCACATCACCAACTGCGACGGCGCCGTGCTGAGCGTGGAG |
| | | GAGCTGCTGATCGGCGGCGAGATGATCAAAGCCGGCACCCTGACACT |
| | | GGAGGAGGTGCGGCGCAAGTTCAACAACGGCGAGATCAACTTCAGAT |
| | | CT |
| 104 | Right ZFN- | GTACCAGCAGCTATGGCCGAACGCCCTTTTCAATGCAGAATATGTAT |
| | T2A-Left ZFN | GCGAAACTTCTCCCAAAGCTCTGATCTGTCAAGGCACATACGGACAC |
| | (na) | ACACCGGCGAAAAACCCTTTGCATGTGACATTTGTGGAAGAAAATTC |
| | ZFN-R | GCACTTAAACACAATCTCCTGACTCATACAAAAATACATACAGGCGA |
| | Codon | AAAACCTTTCCAGTGCAGAATCTGTATGCAGAACTTTTCCGACCAAT |
| | diversified | CCAATCTTCGCGCCCACATTAGAACTCACACAGGGGAGAAACCTTTC |
| | Version 5 | GCTTGCGACATATGCGGAAGAAAATTTGCCAGAAATTTTTCACTTAC |
| | ZFN-L | AATGCACACAAAAATACATACTGGGGAAAGAGGGTTTCAATGTCGAA |
| | Not diversified | TCTGTATGAGAAATTTCAGTCTGCGCCATGATCTGGAGAGACATATA |
| | | AGAACACACACAGGAGAGAAACCTTTTGCTTGTGACATATGCGGCCG |
| | | AAAGTTTGCTCATAGATCTAATCTTAACAAACATACAAAGATCCATC |
| | | TTCGGGGTTCACAACTGGTCAAGTCAGAATTGGAAGAGAAAAAATCT |
| | | GAGCTGAGGCACAAATTGAAATACGTTCCTCACGAGTATATTGAACT |
| | | TATCGAGATAGCCCGCAATAGTAGACAAGATAGAATCTTGGAGATGA |
| | | AAGTTATGGAATTCTTTATGAAAGTCTATGGCTATAGGGGAAAACAC |
| | | CTGGGGGGTAGCAGGAAACCTGATGGAGCTATCTATACCGTAGGATC |
| | | ACCTATTGATTATGGAGTAATTGTGGACACTAAGGCATATTCCGGAG |
| | | GATATAATTTGAGTATTGGTCAGGCCGACGAAATGCAACGATACGTG |
| | | AAGGAAAATCAGACCCGCAACAAACACATTAATCCCAATGAATGGTG |
| | | GAAGGTATACCCTAGTAGCGTTACAGAGTTTAAATTCCTTTTCGTCA |
| | | GCGGCCACTTTAAAGGAAATTATAAAGCACAACTCACCAGACTTAAT |
| | | CGAAAAACTAACTGTAACGGCGCCGTACTGTCAGTGGAGGAGCTGCT |
| | | CATTGGAGGCGAGATGATCAAGGCCGGTACTCTCACACTGGAAGAAG |
| | | TTAGAAGAAAGTTCAACAACGGGGAAATTAATTTCGGCAGCGGAGAG |
| | | GGCAGAGGAAGCCTGCTCACCTGCGGTGACGTGGAGGAAAACCCTGG |
| | | CCCTGCCGCTATGGCTGAGAGGCCCTTCCAGTGTCGAATCTGCATGC |
| | | AGAACTTCAGTCAGTCCGGCAACCTGGCCCGCCACATCCGCACCCAC |
| | | ACCGGCGAGAAGCCTTTTGCCTGTGACATTTGTGGGAGGAAATTTGC |
| | | CCTGAAGCAGAACCTGTGTATGCATACCAAGATACACACGGGCGAGA |
| | | AGCCCTTCCAGTGTCGAATCTGCATGCAGAAGTTTGCCTGGCAGTCC |
| | | AACCTGCAGAACCATACCAAGATACACACGGGCGAGAAGCCCTTCCA |
| | | GTGTCGAATCTGCATGCGTAACTTCAGTACCTCCGGCAACCTGACCC |
| | | GCCACATCCGCACCCACACCGGCGAGAAGCCTTTTGCCTGTGACATT |
| | | TGTGGGAGGAAATTTGCCCGCCGCTCCCACCTGACCTCCCATACCAA |
| | | GATACACCTGCGGGGATCCCAGCTGGTGAAGAGCGAGCTGGAGGAGA |
| | | AGAAGTCCGAGCTGCGGCACAAGCTGAAGTACGTGCCCCACGAGTAC |
| | | ATCGAGCTGATCGAGATCGCCAGGAACAGCACCCAGGACCGCATCCT |
| | | GGAGATGAAGGTGATGGAGTTCTTCATGAAGGTGTACGGCTACAGGG |
| | | GAAAGCACCTGGGCGGAAGCAGAAAGCCTGACGGCGCCATCTATACA |
| | | GTGGGCAGCCCCATCGATTACGGCGTGATCGTGGACACAAAGGCCTA |
| | | CAGCGGCGGCTACAATCTGCCTATCGGCCAGGCCGACGAGATGGAGA |
| | | GATACGTGGAGGAGAACCAGACCCGGGATAAGCACCTCAACCCCAAC |
| | | GAGTGGTGGAAGGTGTACCCTAGCAGCGTGACCGAGTTCAAGTTCCT |
| | | GTTCGTGAGCGGCCACTTCAAGGGCAACTACAAGGCCCAGCTGACCA |
| | | GGCTGAACCACATCACCAACTGCGACGGCGCCGTGCTGAGCGTGGAG |
| | | GAGCTGCTGATCGGCGGCGAGATGATCAAAGCCGGCACCCTGACACT |
| | | GGAGGAGGTGCGGCGCAAGTTCAACAACGGCGAGATCAACTTCAGAT |
| | | CT |
| 105 | Right ZFN- | GTTCCCGCTGCTATGGCTGAGAGACCTTTCCAATGTAGGATCTGTAT |
| | T2A-Left ZFN | GCGAAACTTCTCCCAGAGCTCCGACCTGAGTCGCCATATAAGAACCC |
| | (na) | ATACCGGAGAAAAACCATTTGCTTGTGACATTTGTGGCAGAAAGTTC |
| | ZFN-R | GCTCTTAAACACAACCTGCTTACACATACTAAAATACACACAGGGGA |
| | Codon | GAAACCCTTTCAATGCCGGATCTGTATGCAAAACTTTAGCGATCAAT |
| | diversified | CAAACTTGCGAGCCCATATCCGCACTCACACCGGCGAGAAGCCTTTT |
| | Version 6 | GCATGCGATATATGTGGACGGAAATTTGCTAGAAACTTCTCATTGAC |
| | ZFN-L | CATGCATACAAAAATACACACCGGGGAACGAGGATTTCAATGTCGAA |
| | Not diversified | TTTGTATGAGAAATTTTAGCCTTAGGCACGACTTGGAACGGCACATA |
| | | AGAACCCACACCGGAGAGAAGCCTTTTGCTTGTGATATTTGCGGCAG |
| | | AAAGTTCGCCCATCGCAGCAATCTTAACAAGCACACCAAGATTCATT |
| | | TGAGAGGTTCCCAGCTGGTCAAAAGCGAACTTGAAGAAAAGAAATCC |
| | | GAGCTTAGACACAAACTGAAATACGTGCCTCACGAGTATATTGAGCT |
| | | GATTGAAATAGCAAGGAATTCAACACAAGACAGGATCCTCGAAATGA |
| | | AGGTTATGGAGTTTTTCATGAAAGTTTACGGCTACAGAGGGAAGCAT |
| | | CTGGGCGGATCAAGAAAACCAGACGGCGCAATCTACACAGTTGGATC |
| | | CCCAATAGATTACGGAGTGATTGTTGACACCAAGGCTTATTCAGGAG |
| | | GTTACAATCTGTCCATTGGTCAGGCCGATGAAATGCAAAGATATGTT |
| | | AAGGAAAATCAAACTCGAAACAAACACATTAATCCAAACGAATGGTG |
| | | GAAAGTATATCCAAGCTCCGTCACTGAATTTAAATTTTTGTTTGTAT |
| | | CCGGACATTTTAAGGGCAACTATAAGGCTCAACTGACCAGACTGAAT |
| | | AGGAAGACCAATTGTAACGGAGCTGTACTCAGCGTGGAAGAACTGCT |
| | | TATTGGAGGCGAAATGATTAAGGCTGGCACACTTACACTCGAAGAAG |
| | | TTAGAAGAAAATTCAACAATGGTGAGATAAACTTCGGCAGCGGAGAG |
| | | GGCAGAGGAAGCCTGCTCACCTGCGGTGACGTGGAGGAAAACCCTGG |
| | | CCCTGCCGCTATGGCTGAGAGGCCCTTCCAGTGTCGAATCTGCATGC |
| | | AGAACTTCAGTCAGTCCGGCAACCTGGCCCGCCACATCCGCACCCAC |
| | | ACCGGCGAGAAGCCTTTTGCCTGTGACATTTGTGGGAGGAAATTTGC |
| | | CCTGAAGCAGAACCTGTGTATGCATACCAAGATACACACGGGCGAGA |
| | | AGCCCTTCCAGTGTCGAATCTGCATGCAGAAGTTTGCCTGGCAGTCC |
| | | AACCTGCAGAACCATACCAAGATACACACGGGCGAGAAGCCCTTCCA |
| | | GTGTCGAATCTGCATGCGTAACTTCAGTACCTCCGGCAACCTGACCC |
| | | GCCACATCCGCACCCACACCGGCGAGAAGCCTTTTGCCTGTGACATT |
| | | TGTGGGAGGAAATTTGCCCGCCGCTCCCACCTGACCTCCCATACCAA |
| | | GATACACCTGCGGGGATCCCAGCTGGTGAAGAGCGAGCTGGAGGAGA |
| | | AGAAGTCCGAGCTGCGGCACAAGCTGAAGTACGTGCCCCACGAGTAC |
| | | ATCGAGCTGATCGAGATCGCCAGGAACAGCACCCAGGACCGCATCCT |
| | | GGAGATGAAGGTGATGGAGTTCTTCATGAAGGTGTACGGCTACAGGG |
| | | GAAAGCACCTGGGCGGAAGCAGAAAGCCTGACGGCGCCATCTATACA |
| | | GTGGGCAGCCCCATCGATTACGGCGTGATCGTGGACACAAAGGCCTA |
| | | CAGCGGCGGCTACAATCTGCCTATCGGCCAGGCCGACGAGATGGAGA |
| | | GATACGTGGAGGAGAACCAGACCCGGGATAAGCACCTCAACCCCAAC |
| | | GAGTGGTGGAAGGTGTACCCTAGCAGCGTGACCGAGTTCAAGTTCCT |
| | | GTTCGTGAGCGGCCACTTCAAGGGCAACTACAAGGCCCAGCTGACCA |
| | | GGCTGAACCACATCACCAACTGCGACGGCGCCGTGCTGAGCGTGGAG |
| | | GAGCTGCTGATCGGCGGCGAGATGATCAAAGCCGGCACCCTGACACT |
| | | GGAGGAGGTGCGGCGCAAGTTCAACAACGGCGAGATCAACTTCAGAT |
| | | CT |
| 106 | Right ZFN- | GTACCCGCCGCTATGGCTGAGAGGCCCTTCCAGTGTCGAATCTGCAT |
| | T2A-Left ZFN | GCGTAACTTCAGTCAGTCCTCCGACCTGTCCCGCCACATCCGCACCC |
| | (na) | ACACCGGCGAGAAGCCTTTTGCCTGTGACATTTGTGGGAGGAAATTT |
| | ZFN-R | GCCCTGAAGCACAACCTGCTGACCCATACCAAGATACACACGGGCGA |
| | Not diversified | GAAGCCCTTCCAGTGTCGAATCTGCATGCAGAACTTCAGTGACCAGT |
| | ZFN-L | CCAACCTGCGCGCCCACATCCGCACCCACACCGGCGAGAAGCCTTTT |
| | Not diversified | GCCTGTGACATTTGTGGGAGGAAATTTGCCCGCAACTTCTCCCTGAC |
| | | CATGCATACCAAGATACACACCGGAGAGCGCGGCTTCCAGTGTCGAA |
| | | TCTGCATGCGTAACTTCAGTCTGCGCCACGACCTGGAGCGCCACATC |
| | | CGCACCCACACCGGCGAGAAGCCTTTTGCCTGTGACATTTGTGGGAG |
| | | GAAATTTGCCCACCGCTCCAACCTGAACAAGCATACCAAGATACACC |
| | | TGCGGGGATCCCAGCTGGTGAAGAGCGAGCTGGAGGAGAAGAAGTCC |
| | | GAGCTGCGGCACAAGCTGAAGTACGTGCCCCACGAGTACATCGAGCT |
| | | GATCGAGATCGCCAGGAACAGCACCCAGGACCGCATCCTGGAGATGA |
| | | AGGTGATGGAGTTCTTCATGAAGGTGTACGGCTACAGGGGAAAGCAC |
| | | CTGGGCGGAAGCAGAAAGCCTGACGGCGCCATCTATACAGTGGGCAG |
| | | CCCCATCGATTACGGCGTGATCGTGGACACAAAGGCCTACAGCGGCG |
| | | GCTACAATCTGAGCATCGGCCAGGCCGACGAGATGCAGAGATACGTG |
| | | AAGGAGAACCAGACCCGGAATAAGCACATCAACCCCAACGAGTGGTG |
| | | GAAGGTGTACCCTAGCAGCGTGACCGAGTTCAAGTTCCTGTTCGTGA |
| | | GCGGCCACTTCAAGGGCAACTACAAGGCCCAGCTGACCAGGCTGAAC |
| | | CGCAAAACCAACTGCAATGGCGCCGTGCTGAGCGTGGAGGAGCTGCT |
| | | GATCGGCGGCGAGATGATCAAAGCCGGCACCCTGACACTGGAGGAGG |
| | | TGCGGCGCAAGTTCAACAACGGCGAGATCAACTTCGGCAGCGGAGAG |
| | | GGCAGAGGAAGCCTGCTCACCTGCGGTGACGTGGAGGAAAACCCTGG |
| | | CCCTGCCGCTATGGCTGAGAGGCCCTTCCAGTGTCGAATCTGCATGC |
| | | AGAACTTCAGTCAGTCCGGCAACCTGGCCCGCCACATCCGCACCCAC |
| | | ACCGGCGAGAAGCCTTTTGCCTGTGACATTTGTGGGAGGAAATTTGC |
| | | CCTGAAGCAGAACCTGTGTATGCATACCAAGATACACACGGGCGAGA |
| | | AGCCCTTCCAGTGTCGAATCTGCATGCAGAAGTTTGCCTGGCAGTCC |
| | | AACCTGCAGAACCATACCAAGATACACACGGGCGAGAAGCCCTTCCA |
| | | GTGTCGAATCTGCATGCGTAACTTCAGTACCTCCGGCAACCTGACCC |
| | | GCCACATCCGCACCCACACCGGCGAGAAGCCTTTTGCCTGTGACATT |
| | | TGTGGGAGGAAATTTGCCCGCCGCTCCCACCTGACCTCCCATACCAA |
| | | GATACACCTGCGGGGATCCCAGCTGGTGAAGAGCGAGCTGGAGGAGA |
| | | AGAAGTCCGAGCTGCGGCACAAGCTGAAGTACGTGCCCCACGAGTAC |
| | | ATCGAGCTGATCGAGATCGCCAGGAACAGCACCCAGGACCGCATCCT |
| | | GGAGATGAAGGTGATGGAGTTCTTCATGAAGGTGTACGGCTACAGGG |
| | | GAAAGCACCTGGGCGGAAGCAGAAAGCCTGACGGCGCCATCTATACA |
| | | GTGGGCAGCCCCATCGATTACGGCGTGATCGTGGACACAAAGGCCTA |
| | | CAGCGGCGGCTACAATCTGCCTATCGGCCAGGCCGACGAGATGGAGA |
| | | GATACGTGGAGGAGAACCAGACCCGGGATAAGCACCTCAACCCCAAC |
| | | GAGTGGTGGAAGGTGTACCCTAGCAGCGTGACCGAGTTCAAGTTCCT |
| | | GTTCGTGAGCGGCCACTTCAAGGGCAACTACAAGGCCCAGCTGACCA |
| | | GGCTGAACCACATCACCAACTGCGACGGCGCCGTGCTGAGCGTGGAG |
| | | GAGCTGCTGATCGGCGGCGAGATGATCAAAGCCGGCACCCTGACACT |
| | | GGAGGAGGTGCGGCGCAAGTTCAACAACGGCGAGATCAACTTCAGAT |
| | | CT |
| 107 | Left ZFN-T2A- | GCAGCAATGGCCGAACGACCCTTCCAATGCAGAATATGTATGCAGAA |
| | Right ZFN (na) | TTTTTCTCAGAGCGGGAACCTGGCGAGGCACATAAGAACCCATACAG |
| | ZFN-L | GAGAGAAGCCATTCGCATGCGATATTTGCGGTAGAAAATTTGCACTC |
| | Codon | AAACAAAATCTCTGTATGCACACTAAAATCCATACAGGTGAAAAGCC |
| | diversified | TTTTCAGTGCAGGATTTGTATGCAAAAATTTGCTTGGCAAAGTAACT |
| | Version 1 | TGCAGAACCACACAAAGATACACACAGGAGAGAAACCCTTCCAATGC |
| | ZFN-R | CGAATCTGTATGCGCAACTTCAGTACATCCGGAAATTTGACTAGACA |
| | Not diversified | TATTAGGACCCACACCGGCGAGAAGCCATTTGCCTGCGATATTTGTG |
| | | GACGGAAATTCGCACGACGCAGCCATCTGACCAGTCATACTAAGATT |
| | | CATCTCCGCGGCAGCCAGCTTGTGAAGTCCGAACTGGAGGAAAAGAA |
| | | GAGCGAACTGCGCCACAAATTGAAATACGTTCCGCATGAGTACATAG |
| | | AGCTCATTGAAATCGCTAGAAACTCTACCCAAGACAGGATACTGGAA |
| | | ATGAAAGTGATGGAATTTTTCATGAAAGTTTATGGTTATAGGGGCAA |
| | | ACATCTGGGTGGCTCTCGCAAGCCCGATGGGGCCATTTATACTGTCG |
| | | GCTCACCTATCGACTATGGCGTCATTGTGGATACCAAGGCTTATTCT |
| | | GGAGGATACAACCTGCCCATCGGACAAGCAGACGAAATGGAAAGATA |
| | | CGTCGAGGAGAATCAAACCCGAGACAAGCATCTGAACCCAAACGAGT |
| | | GGTGGAAAGTGTACCCGAGCAGCGTTACTGAGTTCAAATTTCTCTTT |
| | | GTAAGCGGACATTTTAAAGGGAATTACAAAGCACAACTGACTAGGCT |
| | | GAACCATATAACCAACTGTGACGGGGCCGTATTGAGTGTGGAAGAGC |
| | | TTCTGATTGGAGGAGAGATGATTAAGGCTGGCACACTGACTCTCGAA |
| | | GAAGTGAGGCGCAAATTCAATAACGGTGAAATCAACTTCCGGTCTGG |
| | | CAGCGGAGAGGGCAGAGGAAGCCTGCTCACCTGCGGTGACGTGGAGG |
| | | AAAACCCTGGCCCTGTACCCGCCGCTATGGCTGAGAGGCCCTTCCAG |
| | | TGTCGAATCTGCATGCGTAACTTCAGTCAGTCCTCCGACCTGTCCCG |
| | | CCACATCCGCACCCACACCGGCGAGAAGCCTTTTGCCTGTGACATTT |
| | | GTGGGAGGAAATTTGCCCTGAAGCACAACCTGCTGACCCATACCAAG |
| | | ATACACACGGGCGAGAAGCCCTTCCAGTGTCGAATCTGCATGCAGAA |
| | | CTTCAGTGACCAGTCCAACCTGCGCGCCCACATCCGCACCCACACCG |
| | | GCGAGAAGCCTTTTGCCTGTGACATTTGTGGGAGGAAATTTGCCCGC |
| | | AACTTCTCCCTGACCATGCATACCAAGATACACACCGGAGAGCGCGG |
| | | CTTCCAGTGTCGAATCTGCATGCGTAACTTCAGTCTGCGCCACGACC |
| | | TGGAGCGCCACATCCGCACCCACACCGGCGAGAAGCCTTTTGCCTGT |
| | | GACATTTGTGGGAGGAAATTTGCCCACCGCTCCAACCTGAACAAGCA |
| | | TACCAAGATACACCTGCGGGGATCCCAGCTGGTGAAGAGCGAGCTGG |
| | | AGGAGAAGAAGTCCGAGCTGCGGCACAAGCTGAAGTACGTGCCCCAC |
| | | GAGTACATCGAGCTGATCGAGATCGCCAGGAACAGCACCCAGGACCG |
| | | CATCCTGGAGATGAAGGTGATGGAGTTCTTCATGAAGGTGTACGGCT |
| | | ACAGGGGAAAGCACCTGGGCGGAAGCAGAAAGCCTGACGGCGCCATC |
| | | TATACAGTGGGCAGCCCCATCGATTACGGCGTGATCGTGGACACAAA |
| | | GGCCTACAGCGGCGGCTACAATCTGAGCATCGGCCAGGCCGACGAGA |
| | | TGCAGAGATACGTGAAGGAGAACCAGACCCGGAATAAGCACATCAAC |
| | | CCCAACGAGTGGTGGAAGGTGTACCCTAGCAGCGTGACCGAGTTCAA |
| | | GTTCCTGTTCGTGAGCGGCCACTTCAAGGGCAACTACAAGGCCCAGC |
| | | TGACCAGGCTGAACCGCAAAACCAACTGCAATGGCGCCGTGCTGAGC |
| | | GTGGAGGAGCTGCTGATCGGCGGCGAGATGATCAAAGCCGGCACCCT |
| | | GACACTGGAGGAGGTGCGGCGCAAGTTCAACAACGGCGAGATCAACT |
| | | TC |
| 108 | Left ZFN-T2A- | GCCGCCATGGCAGAGAGACCCTTTCAATGTAGAATCTGTATGCAAAA |
| | Right ZFN (na) | TTTCTCTCAGAGTGGTAACCTTGCAAGACACATCAGAACTCATACAG |
| | ZFN-L | GTGAGAAGCCGTTTGCATGTGACATTTGCGGTAGGAAATTTGCCTTG |
| | Codon | AAACAGAATCTTTGTATGCACACAAAAATCCATACTGGTGAAAAGCC |
| | diversified | ATTCCAATGCCGCATCTGTATGCAAAAATTCGCGTGGCAGTCCAATT |
| | Version 2 | TGCAGAACCATACCAAGATTCACACGGGAGAAAAACCATTTCAGTGC |
| | ZFN-R | CGCATCTGCATGCGCAACTTTTCTACATCAGGAAACCTTACACGACA |
| | Not diversified | TATTCGGACGCACACTGGAGAAAAACCATTTGCTTGTGACATATGCG |
| | | GCCGAAAATTTGCCAGACGCTCTCATCTCACCTCACATACTAAGATT |
| | | CATTTGCGCGGAAGTCAGCTGGTGAAGAGTGAATTGGAAGAAAAAAA |
| | | GTCAGAGCTGAGACACAAACTGAAATATGTTCCACACGAGTACATCG |
| | | AGCTTATCGAGATAGCAAGAAACTCCACCCAGGACAGAATTTTGGAA |
| | | ATGAAAGTTATGGAATTCTTTATGAAAGTGTATGGCTACAGGGGTAA |
| | | ACATCTGGGGGGATCAAGAAAGCCTGATGGTGCAATTTACACAGTGG |
| | | GCTCTCCTATCGACTACGGTGTGATCGTGGATACAAAGGCCTACTCT |
| | | GGAGGATATAATTTGCCTATTGGACAAGCCGATGAAATGGAAAGATA |
| | | TGTGGAGGAAAACCAGACTCGCGATAAGCACCTGAACCCAAATGAAT |
| | | GGTGGAAAGTGTACCCTTCATCTGTTACCGAATTTAAATTTTTGTTC |
| | | GTTTCCGGGCATTTCAAGGGGAACTACAAGGCACAGCTGACGAGACT |
| | | GAATCACATCACGAACTGCGACGGCGCTGTACTGTCCGTGGAAGAGC |
| | | TTTTGATCGGGGGCGAAATGATTAAGGCCGGCACACTGACGCTGGAG |
| | | GAGGTGCGGCGAAAATTTAATAATGGCGAGATCAATTTTAGGAGTGG |
| | | CAGCGGAGAGGGCAGAGGAAGCCTGCTCACCTGCGGTGACGTGGAGG |
| | | AAAACCCTGGCCCTGTACCCGCCGCTATGGCTGAGAGGCCCTTCCAG |
| | | TGTCGAATCTGCATGCGTAACTTCAGTCAGTCCTCCGACCTGTCCCG |
| | | CCACATCCGCACCCACACCGGCGAGAAGCCTTTTGCCTGTGACATTT |
| | | GTGGGAGGAAATTTGCCCTGAAGCACAACCTGCTGACCCATACCAAG |
| | | ATACACACGGGCGAGAAGCCCTTCCAGTGTCGAATCTGCATGCAGAA |
| | | CTTCAGTGACCAGTCCAACCTGCGCGCCCACATCCGCACCCACACCG |
| | | GCGAGAAGCCTTTTGCCTGTGACATTTGTGGGAGGAAATTTGCCCGC |
| | | AACTTCTCCCTGACCATGCATACCAAGATACACACCGGAGAGCGCGG |
| | | CTTCCAGTGTCGAATCTGCATGCGTAACTTCAGTCTGCGCCACGACC |
| | | TGGAGCGCCACATCCGCACCCACACCGGCGAGAAGCCTTTTGCCTGT |
| | | GACATTTGTGGGAGGAAATTTGCCCACCGCTCCAACCTGAACAAGCA |
| | | TACCAAGATACACCTGCGGGGATCCCAGCTGGTGAAGAGCGAGCTGG |
| | | AGGAGAAGAAGTCCGAGCTGCGGCACAAGCTGAAGTACGTGCCCCAC |
| | | GAGTACATCGAGCTGATCGAGATCGCCAGGAACAGCACCCAGGACCG |
| | | CATCCTGGAGATGAAGGTGATGGAGTTCTTCATGAAGGTGTACGGCT |
| | | ACAGGGGAAAGCACCTGGGCGGAAGCAGAAAGCCTGACGGCGCCATC |
| | | TATACAGTGGGCAGCCCCATCGATTACGGCGTGATCGTGGACACAAA |
| | | GGCCTACAGCGGCGGCTACAATCTGAGCATCGGCCAGGCCGACGAGA |
| | | TGCAGAGATACGTGAAGGAGAACCAGACCCGGAATAAGCACATCAAC |
| | | CCCAACGAGTGGTGGAAGGTGTACCCTAGCAGCGTGACCGAGTTCAA |
| | | GTTCCTGTTCGTGAGCGGCCACTTCAAGGGCAACTACAAGGCCCAGC |
| | | TGACCAGGCTGAACCGCAAAACCAACTGCAATGGCGCCGTGCTGAGC |
| | | GTGGAGGAGCTGCTGATCGGCGGCGAGATGATCAAAGCCGGCACCCT |
| | | GACACTGGAGGAGGTGCGGCGCAAGTTCAACAACGGCGAGATCAACT |
| | | TC |
| 109 | Left ZFN-T2A- | GCCGCGATGGCAGAGAGACCATTTCAGTGTAGAATCTGTATGCAGAA |
| | Right ZFN (na) | CTTTTCCCAATCAGGAAACCTGGCACGACACATTAGAACCCATACTG |
| | ZFN-L | GAGAAAAGCCGTTCGCTTGCGACATTTGCGGTAGAAAATTTGCTTTG |
| | Codon | AAACAGAACTTGTGTATGCATACCAAGATTCATACCGGCGAAAAACC |
| | diversified | ATTTCAATGCAGGATTTGTATGCAGAAGTTCGCCTGGCAATCCAATT |
| | Version 3 | TGCAGAATCATACTAAAATTCATACCGGAGAAAAACCATTCCAATGC |
| | ZFN-R | CGCATTTGTATGAGAAACTTTTCTACCTCTGGCAATCTCACCAGACA |
| | Not diversified | TATCAGAACACACACAGGCGAGAAACCGTTCGCATGCGATATCTGTG |
| | | GGCGAAAGTTTGCCAGAAGATCCCATCTCACATCACATACTAAAATA |
| | | CATTTGCGAGGAAGTCAACTGGTCAAGTCCGAACTGGAGGAAAAAAA |
| | | AAGTGAGCTGCGACACAAGTTGAAGTACGTACCACACGAATACATCG |
| | | AGCTGATTGAGATAGCACGGAACTCTAGCCAGGATAGAATACTGGAG |
| | | ATGAAAGTTATGGAATTCTTTATGAAGGTGTACGGATACAGGGGGAA |
| | | GCATCTTGGCGGGAGCCGGAAACCAGACGGAGCAATCTATACCGTCG |
| | | GGTCACCTATAGACTATGGAGTTATTGTCGATACAAAGGCCTATTCA |
| | | GGAGGTTATAATCTGCCAATCGGCCAAGCCGACGAGATGGAGAGGTA |
| | | CGTGGAGGAAAATCAGACCAGAGACAAGCACCTGAACCCTAATGAAT |
| | | GGTGGAAAGTGTACCCTAGCAGCGTCACTGAGTTCAAATTCCTGTTC |
| | | GTCAGCGGTCATTTTAAAGGAAATTATAAAGCCCAGCTCACTAGACT |
| | | CAACCATATTACAAACTGCGACGGAGCCGTACTTAGCGTTGAAGAGT |
| | | TGCTTATCGGAGGAGAGATGATCAAAGCCGGAACCCTCACACTTGAA |
| | | GAAGTGCGAAGAAAATTCAATAACGGAGAGATAAATTTTAGGAGTGG |
| | | CAGCGGAGAGGGCAGAGGAAGCCTGCTCACCTGCGGTGACGTGGAGG |
| | | AAAACCCTGGCCCTGCCGCTATGGCTGAGAGGCCCTTCCAGTGTCGA |
| | | ATCTGCATGCAGAACTTCAGTCAGTCCGGCAACCTGGCCCGCCACAT |
| | | CCGCACCCACACCGGCGAGAAGCCTTTTGCCTGTGACATTTGTGGGA |
| | | GGAAATTTGCCCTGAAGCAGAACCTGTGTATGCATACCAAGATACAC |
| | | ACGGGCGAGAAGCCCTTCCAGTGTCGAATCTGCATGCAGAAGTTTGC |
| | | CTGGCAGTCCAACCTGCAGAACCATACCAAGATACACACGGGCGAGA |
| | | AGCCCTTCCAGTGTCGAATCTGCATGCGTAACTTCAGTACCTCCGGC |
| | | AACCTGACCCGCCACATCCGCACCCACACCGGCGAGAAGCCTTTTGC |
| | | CTGTGACATTTGTGGGAGGAAATTTGCCCGCCGCTCCCACCTGACCT |
| | | CCCATACCAAGATACACCTGCGGGGATCCCAGCTGGTGAAGAGCGAG |
| | | CTGGAGGAGAAGAAGTCCGAGCTGCGGCACAAGCTGAAGTACGTGCC |
| | | CCACGAGTACATCGAGCTGATCGAGATCGCCAGGAACAGCACCCAGG |
| | | ACCGCATCCTGGAGATGAAGGTGATGGAGTTCTTCATGAAGGTGTAC |
| | | GGCTACAGGGGAAAGCACCTGGGCGGAAGCAGAAAGCCTGACGGCGC |
| | | CATCTATACAGTGGGCAGCCCCATCGATTACGGCGTGATCGTGGACA |
| | | CAAAGGCCTACAGCGGCGGCTACAATCTGCCTATCGGCCAGGCCGAC |
| | | GAGATGGAGAGATACGTGGAGGAGAACCAGACCCGGGATAAGCACCT |
| | | CAACCCCAACGAGTGGTGGAAGGTGTACCCTAGCAGCGTGACCGAGT |
| | | TCAAGTTCCTGTTCGTGAGCGGCCACTTCAAGGGCAACTACAAGGCC |
| | | CAGCTGACCAGGCTGAACCACATCACCAACTGCGACGGCGCCGTGCT |
| | | GAGCGTGGAGGAGCTGCTGATCGGCGGCGAGATGATCAAAGCCGGCA |
| | | CCCTGACACTGGAGGAGGTGCGGCGCAAGTTCAACAACGGCGAGATC |
| | | AACTTCAGATCT |
| 110 | Left ZFN-T2A- | GCAGCAATGGCCGAGAGACCTTTTCAGTGCAGGATTTGTATGCAAAA |
| | Right ZFN (na) | CTTCTCTCAGTCCGGTAACCTGGCCCGGCACATACGAACACATACCG |
| | ZFN-L | GCGAAAAACCCTTTGCTTGCGACATCTGCGGAAGAAAGTTCGCTCTT |
| | Codon | AAACAGAACCTGTGCATGCATACAAAAATTCATACAGGTGAGAAGCC |
| | diversified | ATTCCAATGCAGAATATGTATGCAGAAATTCGCCTGGCAAAGCAACC |
| | Version 4 | TGCAAAACCACACTAAGATCCACACAGGGGAAAAGCCTTTTCAATGT |
| | ZFN-R | AGAATCTGTATGAGAAACTTTAGTACATCCGGAAATCTCACACGACA |
| | Not diversified | TATCAGAACCCACACTGGAGAAAAACCTTTTGCCTGCGACATCTGCG |
| | | GAAGAAAATTCGCCCGAAGGTCCCACTTGACTAGTCATACCAAAATC |
| | | CACTTGCGAGGCTCACAGCTGGTTAAATCCGAACTTGAAGAAAAAAA |
| | | AAGTGAACTGCGGCATAAACTGAAGTATGTCCCCCATGAATATATCG |
| | | AACTGATAGAAATCGCCCGAAATAGCACCCAAGATAGAATCCTCGAA |
| | | ATGAAGGTTATGGAATTTTTCATGAAGGTCTATGGATATAGGGGCAA |
| | | GCACCTTGGCGGATCCCGGAAACCTGATGGAGCTATCTACACAGTGG |
| | | GCTCACCAATAGACTATGGAGTTATCGTCGATACAAAAGCATACAGC |
| | | GGAGGATACAATTTGCCAATAGGTCAAGCAGATGAGATGGAAAGATA |
| | | CGTGGAGGAAAACCAAACAAGAGATAAGCATCTGAACCCCAACGAAT |
| | | GGTGGAAAGTGTACCCCAGTTCTGTAACCGAATTTAAGTTCTTGTTC |
| | | GTTTCAGGTCACTTCAAGGGTAATTACAAGGCTCAACTGACTAGACT |
| | | CAACCATATTACAAATTGCGATGGTGCTGTGCTTTCCGTGGAAGAAT |
| | | TGCTGATTGGTGGAGAGATGATAAAAGCTGGTACCCTCACCTTGGAA |
| | | GAAGTGCGCAGAAAATTCAATAATGGCGAGATCAACTTCCGAAGTGG |
| | | CAGCGGAGAGGGCAGAGGAAGCCTGCTCACCTGCGGTGACGTGGAGG |
| | | AAAACCCTGGCCCTGTACCCGCCGCTATGGCTGAGAGGCCCTTCCAG |
| | | TGTCGAATCTGCATGCGTAACTTCAGTCAGTCCTCCGACCTGTCCCG |
| | | CCACATCCGCACCCACACCGGCGAGAAGCCTTTTGCCTGTGACATTT |
| | | GTGGGAGGAAATTTGCCCTGAAGCACAACCTGCTGACCCATACCAAG |
| | | ATACACACGGGCGAGAAGCCCTTCCAGTGTCGAATCTGCATGCAGAA |
| | | CTTCAGTGACCAGTCCAACCTGCGCGCCCACATCCGCACCCACACCG |
| | | GCGAGAAGCCTTTTGCCTGTGACATTTGTGGGAGGAAATTTGCCCGC |
| | | AACTTCTCCCTGACCATGCATACCAAGATACACACCGGAGAGCGCGG |
| | | CTTCCAGTGTCGAATCTGCATGCGTAACTTCAGTCTGCGCCACGACC |
| | | TGGAGCGCCACATCCGCACCCACACCGGCGAGAAGCCTTTTGCCTGT |
| | | GACATTTGTGGGAGGAAATTTGCCCACCGCTCCAACCTGAACAAGCA |
| | | TACCAAGATACACCTGCGGGGATCCCAGCTGGTGAAGAGCGAGCTGG |
| | | AGGAGAAGAAGTCCGAGCTGCGGCACAAGCTGAAGTACGTGCCCCAC |
| | | GAGTACATCGAGCTGATCGAGATCGCCAGGAACAGCACCCAGGACCG |
| | | CATCCTGGAGATGAAGGTGATGGAGTTCTTCATGAAGGTGTACGGCT |
| | | ACAGGGGAAAGCACCTGGGCGGAAGCAGAAAGCCTGACGGCGCCATC |
| | | TATACAGTGGGCAGCCCCATCGATTACGGCGTGATCGTGGACACAAA |
| | | GGCCTACAGCGGCGGCTACAATCTGAGCATCGGCCAGGCCGACGAGA |
| | | TGCAGAGATACGTGAAGGAGAACCAGACCCGGAATAAGCACATCAAC |
| | | CCCAACGAGTGGTGGAAGGTGTACCCTAGCAGCGTGACCGAGTTCAA |
| | | GTTCCTGTTCGTGAGCGGCCACTTCAAGGGCAACTACAAGGCCCAGC |
| | | TGACCAGGCTGAACCGCAAAACCAACTGCAATGGCGCCGTGCTGAGC |
| | | GTGGAGGAGCTGCTGATCGGCGGCGAGATGATCAAAGCCGGCACCCT |
| | | GACACTGGAGGAGGTGCGGCGCAAGTTCAACAACGGCGAGATCAACT |
| | | TC |
| ill | Left ZFN-T2A- | GCAGCAATGGCAGAGAGACCATTTCAGTGCAGAATATGTATGCAAAA |
| | Right ZFN (na) | CTTCTCCCAGAGCGGTAATCTGGCTAGGCATATTAGAACACACACCG |
| | ZFN-L | GGGAAAAACCTTTCGCTTGCGATATATGTGGTAGAAAGTTCGCCCTC |
| | Codon | AAACAGAATCTGTGCATGCACACTAAAATCCATACAGGAGAAAAGCC |
| | diversified | CTTTCAGTGTAGAATTTGTATGCAGAAATTTGCTTGGCAGTCAAATT |
| | Version 5 | TGCAAAATCACACCAAAATACACACAGGAGAAAAACCATTTCAGTGT |
| | ZFN-R | AGAATATGTATGAGAAATTTTTCCACTTCCGGAAATCTGACCAGACA |
| | Not diversified | TATACGGACACACACTGGGGAAAAGCCCTTCGCTTGCGACATCTGCG |
| | | GAAGAAAGTTCGCTAGACGGTCCCACTTGACATCCCACACTAAGATA |
| | | CATCTTCGCGGTAGCCAACTGGTGAAAAGTGAACTGGAGGAAAAAAA |
| | | ATCTGAGCTGAGACATAAACTGAAATACGTACCACATGAATACATAG |
| | | AACTTATAGAAATAGCTAGGAACTCCACCCAGGACAGAATACTTGAA |
| | | ATGAAGGTCATGGAGTTTTTTATGAAAGTTTACGGATACAGGGGCAA |
| | | ACACCTTGGAGGGTCTCGGAAGCCTGATGGCGCAATTTATACCGTGG |
| | | GTAGCCCTATAGATTATGGAGTGATTGTGGATACAAAGGCTTACAGT |
| | | GGCGGCTATAATTTGCCTATCGGACAGGCCGATGAGATGGAAAGATA |
| | | CGTTGAAGAAAACCAAACACGAGATAAGCATCTGAACCCCAATGAAT |
| | | GGTGGAAAGTGTATCCTTCAAGCGTTACCGAGTTTAAGTTCCTCTTC |
| | | GTTTCTGGGCATTTCAAGGGCAACTACAAAGCTCAGCTTACAAGACT |
| | | CAACCACATAACCAATTGTGATGGAGCAGTCCTCAGCGTGGAAGAAC |
| | | TCCTTATTGGGGGTGAGATGATTAAAGCAGGGACCCTTACTCTTGAA |
| | | GAGGTTAGAAGAAAATTCAATAACGGAGAGATTAATTTTAGAAGTGG |
| | | CAGCGGAGAGGGCAGAGGAAGCCTGCTCACCTGCGGTGACGTGGAGG |
| | | AAAACCCTGGCCCTGTACCCGCCGCTATGGCTGAGAGGCCCTTCCAG |
| | | TGTCGAATCTGCATGCGTAACTTCAGTCAGTCCTCCGACCTGTCCCG |
| | | CCACATCCGCACCCACACCGGCGAGAAGCCTTTTGCCTGTGACATTT |
| | | GTGGGAGGAAATTTGCCCTGAAGCACAACCTGCTGACCCATACCAAG |
| | | ATACACACGGGCGAGAAGCCCTTCCAGTGTCGAATCTGCATGCAGAA |
| | | CTTCAGTGACCAGTCCAACCTGCGCGCCCACATCCGCACCCACACCG |
| | | GCGAGAAGCCTTTTGCCTGTGACATTTGTGGGAGGAAATTTGCCCGC |
| | | AACTTCTCCCTGACCATGCATACCAAGATACACACCGGAGAGCGCGG |
| | | CTTCCAGTGTCGAATCTGCATGCGTAACTTCAGTCTGCGCCACGACC |
| | | TGGAGCGCCACATCCGCACCCACACCGGCGAGAAGCCTTTTGCCTGT |
| | | GACATTTGTGGGAGGAAATTTGCCCACCGCTCCAACCTGAACAAGCA |
| | | TACCAAGATACACCTGCGGGGATCCCAGCTGGTGAAGAGCGAGCTGG |
| | | AGGAGAAGAAGTCCGAGCTGCGGCACAAGCTGAAGTACGTGCCCCAC |
| | | GAGTAGATCGAGCTGATCGAGATCGCCAGGAACAGCACCCAGGACCG |
| | | CATCCTGGAGATGAAGGTGATGGAGTTCTTCATGAAGGTGTACGGCT |
| | | ACAGGGGAAAGCACCTGGGCGGAAGCAGAAAGCCTGACGGCGCCATC |
| | | TATACAGTGGGCAGCCCCATCGATTACGGCGTGATCGTGGACACAAA |
| | | GGCCTACAGCGGCGGCTACAATCTGAGCATCGGCCAGGCCGACGAGA |
| | | TGCAGAGATACGTGAAGGAGAACCAGACCCGGAATAAGCACATCAAC |
| | | CCCAACGAGTGGTGGAAGGTGTACCCTAGCAGCGTGACCGAGTTCAA |
| | | GTTCCTGTTCGTGAGCGGCCACTTCAAGGGCAACTACAAGGCCCAGC |
| | | TGACCAGGCTGAACCGCAAAACCAACTGCAATGGCGCCGTGCTGAGC |
| | | GTGGAGGAGCTGCTGATCGGCGGCGAGATGATCAAAGCCGGCACCCT |
| | | GACACTGGAGGAGGTGCGGCGCAAGTTCAACAACGGCGAGATCAACT |
| | | TC |
| 112 | Left ZFN-T2A- | GCAGCCATGGCCGAACGCCCATTTCAATGTAGAATTTGTATGCAGAA |
| | Right ZFN (na) | TTTTTCACAATCAGGAAACCTGGCTAGACATATCAGAACACATACTG |
| | ZFN-L | GAGAAAAGCCCTTTGCTTGTGATATCTGTGGAAGGAAATTCGCCCTG |
| | Codon | AAACAAAACCTCTGTATGCACACAAAGATCCACACCGGCGAAAAGCC |
| | diversified | TTTCCAGTGTAGGATATGCATGCAAAAATTCGCCTGGCAGTCCAATC |
| | Version 6 | TGCAGAACCATACCAAAATTCATACTGGTGAAAAGCCATTTCAGTGC |
| | ZFN-R | AGAATATGTATGAGAAACTTTAGCACTTCAGGAAATCTCACAAGACA |
| | Not diversified | TATAAGAACACATACAGGGGAAAAACCTTTTGCTTGCGATATCTGCG |
| | | GCAGGAAATTCGCTCGGAGAAGTCATCTCACAAGCCATACAAAAATC |
| | | CACCTGCGAGGAAGCCAGCTGGTCAAGTCTGAACTGGAAGAAAAAAA |
| | | AAGCGAACTGCGGCATAAACTCAAATACGTCCCACATGAATACATTG |
| | | AGCTCATCGAAATTGCTAGAAACTCTACTCAAGATAGGATATTGGAG |
| | | ATGAAGGTAATGGAATTCTTCATGAAGGTTTATGGATATAGAGGAAA |
| | | ACATCTTGGAGGCAGTAGGAAACCCGATGGCGCTATCTACACCGTAG |
| | | GGAGTCCAATCGACTACGGCGTGATTGTTGACACCAAAGCCTATTCT |
| | | GGAGGGTATAATCTCCCAATTGGACAGGCAGATGAGATGGAAAGATA |
| | | TGTAGAAGAAAATCAGACAAGAGATAAGCACCTTAACCCTAACGAGT |
| | | GGTGGAAAGTGTACCCAAGCAGTGTTACTGAATTTAAATTTCTTTTT |
| | | GTATCAGGACACTTTAAAGGCAATTACAAAGCACAACTGACCAGACT |
| | | CAATCACATTACCAATTGCGACGGAGCCGTACTGAGCGTGGAGGAGT |
| | | TGCTGATCGGAGGCGAAATGATTAAAGCTGGCACTCTGACCCTGGAA |
| | | GAAGTAAGAAGAAAGTTCAATAATGGAGAAATAAACTTTCGCTCCGG |
| | | CAGCGGAGAGGGCAGAGGAAGCCTGCTCACCTGCGGTGACGTGGAGG |
| | | AAAACCCTGGCCCTGTACCCGCCGCTATGGCTGAGAGGCCCTTCCAG |
| | | TGTCGAATCTGCATGCGTAACTTCAGTCAGTCCTCCGACCTGTCCCG |
| | | CCACATCCGCACCCACACCGGCGAGAAGCCTTTTGCCTGTGACATTT |
| | | GTGGGAGGAAATTTGCCCTGAAGCACAACCTGCTGACCCATACCAAG |
| | | ATACACACGGGCGAGAAGCCCTTCCAGTGTCGAATCTGCATGCAGAA |
| | | CTTCAGTGACCAGTCCAACCTGCGCGCCCACATCCGCACCCACACCG |
| | | GCGAGAAGCCTTTTGCCTGTGACATTTGTGGGAGGAAATTTGCCCGC |
| | | AACTTCTCCCTGACCATGCATACCAAGATACACACCGGAGAGCGCGG |
| | | CTTCCAGTGTCGAATCTGCATGCGTAACTTCAGTCTGCGCCACGACC |
| | | TGGAGCGCCACATCCGCACCCACACCGGCGAGAAGCCTTTTGCCTGT |
| | | GACATTTGTGGGAGGAAATTTGCCCACCGCTCCAACCTGAACAAGCA |
| | | TACCAAGATACACCTGCGGGGATCCCAGCTGGTGAAGAGCGAGCTGG |
| | | AGGAGAAGAAGTCCGAGCTGCGGCACAAGCTGAAGTACGTGCCCCAC |
| | | GAGTACATCGAGCTGATCGAGATCGCCAGGAACAGCACCCAGGACCG |
| | | CATCCTGGAGATGAAGGTGATGGAGTTCTTCATGAAGGTGTACGGCT |
| | | ACAGGGGAAAGCACCTGGGCGGAAGCAGAAAGCCTGACGGCGCCATC |
| | | TATACAGTGGGCAGCCCCATCGATTACGGCGTGATCGTGGACACAAA |
| | | GGCCTACAGCGGCGGCTACAATCTGAGCATCGGCCAGGCCGACGAGA |
| | | TGCAGAGATACGTGAAGGAGAACCAGACCCGGAATAAGCACATCAAC |
| | | CCCAACGAGTGGTGGAAGGTGTACCCTAGCAGCGTGACCGAGTTCAA |
| | | GTTCCTGTTCGTGAGCGGCCACTTCAAGGGCAACTACAAGGCCCAGC |
| | | TGACCAGGCTGAACCGCAAAACCAACTGCAATGGCGCCGTGCTGAGC |
| | | GTGGAGGAGCTGCTGATCGGCGGCGAGATGATCAAAGCCGGCACCCT |
| | | GACACTGGAGGAGGTGCGGCGCAAGTTCAACAACGGCGAGATCAACT |
| | | TC |
| 113 | Left ZFN-T2A- | GCCGCTATGGCTGAGAGGCCCTTCCAGTGTCGAATCTGCATGCAGAA |
| | Right ZFN (na) | CTTCAGTCAGTCCGGCAACCTGGCCCGCCACATCCGCACCCACACCG |
| | ZFN-L | GCGAGAAGCCTTTTGCCTGTGACATTTGTGGGAGGAAATTTGCCCTG |
| | Not diversified | AAGCAGAACCTGTGTATGCATACCAAGATACACACGGGCGAGAAGCC |
| | ZFN-R | CTTCCAGTGTCGAATCTGCATGCAGAAGTTTGCCTGGCAGTCCAACC |
| | Not diversified | TGCAGAACCATACCAAGATACACACGGGCGAGAAGCCCTTCCAGTGT |
| | | CGAATCTGCATGCGTAACTTCAGTACCTCCGGCAACCTGACCCGCCA |
| | | CATCCGCACCCACACCGGCGAGAAGCCTTTTGCCTGTGACATTTGTG |
| | | GGAGGAAATTTGCCCGCCGCTCCCACCTGACCTCCCATACCAAGATA |
| | | CACCTGCGGGGATCCCAGCTGGTGAAGAGCGAGCTGGAGGAGAAGAA |
| | | GTCCGAGCTGCGGCACAAGCTGAAGTACGTGCCCCACGAGTACATCG |
| | | AGCTGATCGAGATCGCCAGGAACAGCACCCAGGACCGCATCCTGGAG |
| | | ATGAAGGTGATGGAGTTCTTCATGAAGGTGTACGGCTACAGGGGAAA |
| | | GCACCTGGGCGGAAGCAGAAAGCCTGACGGCGCCATCTATACAGTGG |
| | | GCAGCCCCATCGATTACGGCGTGATCGTGGACACAAAGGCCTACAGC |
| | | GGCGGCTACAATCTGCCTATCGGCCAGGCCGACGAGATGGAGAGATA |
| | | CGTGGAGGAGAACCAGACCCGGGATAAGCACCTCAACCCCAACGAGT |
| | | GGTGGAAGGTGTACCCTAGCAGCGTGACCGAGTTCAAGTTCCTGTTC |
| | | GTGAGCGGCCACTTCAAGGGCAACTACAAGGCCCAGCTGACCAGGCT |
| | | GAACCACATCACCAACTGCGACGGCGCCGTGCTGAGCGTGGAGGAGC |
| | | TGCTGATCGGCGGCGAGATGATCAAAGCCGGCACCCTGACACTGGAG |
| | | GAGGTGCGGCGCAAGTTCAACAACGGCGAGATCAACTTCAGATCTGG |
| | | CAGCGGAGAGGGCAGAGGAAGCCTGCTCACCTGCGGTGACGTGGAGG |
| | | AAAACCCTGGCCCTGTACCCGCCGCTATGGCTGAGAGGCCCTTCCAG |
| | | TGTCGAATCTGCATGCGTAACTTCAGTCAGTCCTCCGACCTGTCCCG |
| | | CCACATCCGCACCCACACCGGCGAGAAGCCTTTTGCCTGTGACATTT |
| | | GTGGGAGGAAATTTGCCCTGAAGCACAACCTGCTGACCCATACCAAG |
| | | ATACACACGGGCGAGAAGCCCTTCCAGTGTCGAATCTGCATGCAGAA |
| | | CTTCAGTGACCAGTCCAACCTGCGCGCCCACATCCGCACCCACACCG |
| | | GCGAGAAGCCTTTTGCCTGTGACATTTGTGGGAGGAAATTTGCCCGC |
| | | AACTTCTCCCTGACCATGCATACCAAGATACACACCGGAGAGCGCGG |
| | | CTTCCAGTGTCGAATCTGCATGCGTAACTTCAGTCTGCGCCACGACC |
| | | TGGAGCGCCACATCCGCACCCACACCGGCGAGAAGCCTTTTGCCTGT |
| | | GACATTTGTGGGAGGAAATTTGCCCACCGCTCCAACCTGAACAAGCA |
| | | TACCAAGATACACCTGCGGGGATCCCAGCTGGTGAAGAGCGAGCTGG |
| | | AGGAGAAGAAGTCCGAGCTGCGGCACAAGCTGAAGTACGTGCCCCAC |
| | | GAGTAGATCGAGCTGATCGAGATCGCCAGGAACAGCACCCAGGACCG |
| | | CATCCTGGAGATGAAGGTGATGGAGTTCTTCATGAAGGTGTACGGCT |
| | | ACAGGGGAAAGCACCTGGGCGGAAGCAGAAAGCCTGACGGCGCCATC |
| | | TATACAGTGGGCAGCCCCATCGATTACGGCGTGATCGTGGACACAAA |
| | | GGCCTACAGCGGCGGCTACAATCTGAGCATCGGCCAGGCCGACGAGA |
| | | TGCAGAGATACGTGAAGGAGAACCAGACCCGGAATAAGCACATCAAC |
| | | CCCAACGAGTGGTGGAAGGTGTACCCTAGCAGCGTGACCGAGTTCAA |
| | | GTTCCTGTTCGTGAGCGGCCACTTCAAGGGCAACTACAAGGCCCAGC |
| | | TGACCAGGCTGAACCGCAAAACCAACTGCAATGGCGCCGTGCTGAGC |
| | | GTGGAGGAGCTGCTGATCGGCGGCGAGATGATCAAAGCCGGCACCCT |
| | | GACACTGGAGGAGGTGCGGCGCAAGTTCAACAACGGCGAGATCAACT |
| | | TC |
| 114 | Left ZFN-T2A- | GCCGCCATGGCAGAGAGACCCTTTCAATGTAGAATCTGTATGCAAAA |
| | Right ZFN (na) | TTTCTCTCAGAGTGGTAACCTTGCAAGACACATCAGAACTCATACAG |
| | ZFN-L | GTGAGAAGCCGTTTGCATGTGACATTTGCGGTAGGAAATTTGCCTTG |
| | Codon | AAACAGAATCTTTGTATGCACACAAAAATCCATACTGGTGAAAAGCC |
| | diversified | ATTCCAATGCCGCATCTGTATGCAAAAATTCGCGTGGCAGTCCAATT |
| | Version 2 | TGCAGAACCATACCAAGATTCACACGGGAGAAAAACCATTTCAGTGC |
| | ZFN-R | CGCATCTGCATGCGCAACTTTTCTACATCAGGAAACCTTACACGACA |
| | Codon | TATTCGGACGCACACTGGAGAAAAACCATTTGCTTGTGACATATGCG |
| | diversified | GCCGAAAATTTGCCAGACGCTCTCATCTCACCTCACATACTAAGATT |
| | Version 4 | CATTTGCGCGGAAGTCAGCTGGTGAAGAGTGAATTGGAAGAAAAAAA |
| | | GTCAGAGCTGAGACACAAACTGAAATATGTTCCACACGAGTACATCG |
| | | AGCTTATCGAGATAGCAAGAAACTCCACCCAGGACAGAATTTTGGAA |
| | | ATGAAAGTTATGGAATTCTTTATGAAAGTGTATGGCTACAGGGGTAA |
| | | ACATCTGGGGGGATCAAGAAAGCCTGATGGTGCAATTTACACAGTGG |
| | | GCTCTCCTATCGACTACGGTGTGATCGTGGATACAAAGGCCTACTCT |
| | | GGAGGATATAATTTGCCTATTGGACAAGCCGATGAAATGGAAAGATA |
| | | TGTGGAGGAAAACCAGACTCGCGATAAGCACCTGAACCCAAATGAAT |
| | | GGTGGAAAGTGTACCCTTCATCTGTTACCGAATTTAAATTTTTGTTC |
| | | GTTTCCGGGCATTTCAAGGGGAACTACAAGGCACAGCTGACGAGACT |
| | | GAATCACATCACGAACTGCGACGGCGCTGTACTGTCCGTGGAAGAGC |
| | | TTTTGATCGGGGGCGAAATGATTAAGGCCGGCACACTGACGCTGGAG |
| | | GAGGTGCGGCGAAAATTTAATAATGGCGAGATCAATTTTAGGAGTGG |
| | | CAGCGGAGAGGGCAGAGGAAGCCTGCTCACCTGCGGTGACGTGGAGG |
| | | AAAACCCTGGCCCTGTACCTGCCGCTATGGCTGAAAGACCTTTCCAG |
| | | TGTAGGATTTGCATGAGAAATTTTTCCCAATCATCCGACCTTTCAAG |
| | | GCATATTAGGACACACACCGGGGAAAAGCCATTTGCTTGTGATATCT |
| | | GCGGGCGCAAATTTGCTCTTAAGCACAATCTTCTTACCCACACCAAA |
| | | ATTCATACAGGAGAAAAACCTTTTCAATGTAGAATCTGCATGCAAAA |
| | | CTTTTCCGATCAGTCAAATCTTAGAGCTCATATCAGAACCCATACCG |
| | | GGGAGAAACCCTTTGCCTGCGACATATGCGGAAGAAAATTTGCTAGG |
| | | AACTTTAGTCTGACCATGCATACCAAAATTCATACCGGCGAACGCGG |
| | | TTTCCAGTGCAGGATTTGTATGAGAAATTTCTCACTGCGGCATGATC |
| | | TTGAAAGACACATACGAACTCATACCGGAGAAAAGCCATTCGCTTGC |
| | | GATATTTGTGGTAGAAAATTTGCCCACAGGTCTAACCTTAATAAGCA |
| | | CACCAAGATTCATCTCAGAGGATCTCAGCTGGTCAAATCAGAACTTG |
| | | AAGAGAAAAAAAGCGAACTGAGACATAAACTGAAGTACGTGCCTCAT |
| | | GAATACATAGAGCTCATTGAAATAGCTAGGAATAGTACACAGGACAG |
| | | GATACTTGAAATGAAGGTAATGGAATTTTTCATGAAGGTTTATGGAT |
| | | ACCGGGGGAAACATCTCGGGGGCAGCAGAAAACCAGACGGAGCAATT |
| | | TATACTGTCGGGAGTCCTATAGATTATGGCGTTATCGTCGATACAAA |
| | | GGCCTATTCCGGTGGGTACAACCTCTCAATTGGTCAGGCTGATGAGA |
| | | TGCAAAGATACGTCAAAGAAAACCAAACCAGAAATAAACATATAAAT |
| | | CCCAATGAATGGTGGAAAGTATACCCAAGTTCCGTGAGTGAATTCAA |
| | | GTTCCTTTTCGTGTCTGGCCACTTTAAAGGAAATTATAAAGCACAAT |
| | | TGACTAGACTGAATAGAAAAACAAACTGTAACGGCGCAGTGCTGTCA |
| | | GTGGAAGAACTGCTCATAGGTGGAGAGATGATCAAGGCCGGGACACT |
| | | TACTCTTGAGGAAGTTAGAAGGAAGTTCAACAACGGCGAAATCAACT |
| | | TT |
| 115 | Right ZFN- | GTACCTGCCGCTATGGCTGAAAGACCTTTCCAGTGTAGGATTTGCAT |
| | T2A-Left ZFN | GAGAAATTTTTCCCAATCATCCGACCTTTCAAGGCATATTAGGACAC |
| | (na) | ACACCGGGGAAAAGCCATTTGCTTGTGATATCTGCGGGCGCAAATTT |
| | ZFN-R | GCTCTTAAGCACAATCTTCTTACCCACACCAAAATTCATACAGGAGA |
| | Codon | AAAACCTTTTCAATGTAGAATCTGCATGCAAAACTTTTCCGATCAGT |
| | diversified | CAAATCTTAGAGCTCATATCAGAACCCATACCGGGGAGAAACCCTTT |
| | Version 4 | GCCTGCGACATATGCGGAAGAAAATTTGCTAGGAACTTTAGTCTGAC |
| | ZFN-L | CATGCATACCAAAATTCATACCGGCGAACGCGGTTTCCAGTGCAGGA |
| | Codon | TTTGTATGAGAAATTTCTCACTGCGGCATGATCTTGAAAGACACATA |
| | diversified | CGAACTCATACCGGAGAAAAGCCATTCGCTTGCGATATTTGTGGTAG |
| | Version 2 | AAAATTTGCCCACAGGTCTAACCTTAATAAGCACACCAAGATTCATC |
| | | TCAGAGGATCTCAGCTGGTCAAATCAGAACTTGAAGAGAAAAAAAGC |
| | | GAACTGAGACATAAACTGAAGTACGTGCCTCATGAATACATAGAGCT |
| | | CATTGAAATAGCTAGGAATAGTACACAGGACAGGATACTTGAAATGA |
| | | AGGTAATGGAATTTTTCATGAAGGTTTATGGATACCGGGGGAAACAT |
| | | CTCGGGGGCAGCAGAAAACCAGACGGAGCAATTTATACTGTCGGGAG |
| | | TCCTATAGATTATGGCGTTATCGTCGATACAAAGGCCTATTCCGGTG |
| | | GGTACAACCTCTCAATTGGTCAGGCTGATGAGATGCAAAGATACGTC |
| | | AAAGAAAACCAAACCAGAAATAAACATATAAATCCCAATGAATGGTG |
| | | GAAAGTATACCCAAGTTCCGTGACTGAATTCAAGTTCCTTTTCGTGT |
| | | CTGGCCACTTTAAAGGAAATTATAAAGCACAATTGACTAGACTGAAT |
| | | AGAAAAACAAACTGTAACGGCGCAGTGCTGTCAGTGGAAGAACTGCT |
| | | CATAGGTGGAGAGATGATCAAGGCCGGGACACTTACTCTTGAGGAAG |
| | | TTAGAAGGAAGTTCAACAACGGCGAAATCAACTTTGGCAGCGGAGAG |
| | | GGCAGAGGAAGCCTGCTCACCTGCGGTGACGTGGAGGAAAACCCTGG |
| | | CCCTGCCGCCATGGCAGAGAGACCCTTTCAATGTAGAATCTGTATGC |
| | | AAAATTTCTCTCAGAGTGGTAACCTTGCAAGACACATCAGAACTCAT |
| | | ACAGGTGAGAAGCCGTTTGCATGTGACATTTGCGGTAGGAAATTTGC |
| | | CTTGAAACAGAATCTTTGTATGCACACAAAAATCCATACTGGTGAAA |
| | | AGCCATTCCAATGCCGCATCTGTATGCAAAAATTCGCGTGGCAGTCC |
| | | AATTTGCAGAACCATACCAAGATTCACACGGGAGAAAAACCATTTCA |
| | | GTGCCGCATCTGCATGCGCAACTTTTCTACATCAGGAAACCTTACAC |
| | | GACATATTCGGACGCACACTGGAGAAAAACCATTTGCTTGTGACATA |
| | | TGCGGCCGAAAATTTGCCAGACGCTCTCATCTCACCTCACATACTAA |
| | | GATTCATTTGCGCGGAAGTCAGCTGGTGAAGAGTGAATTGGAAGAAA |
| | | AAAAGTCAGAGCTGAGACACAAACTGAAATATGTTCCACACGAGTAC |
| | | ATCGAGCTTATCGAGATAGCAAGAAACTCCACCCAGGACAGAATTTT |
| | | GGAAATGAAAGTTATGGAATTCTTTATGAAAGTGTATGGCTACAGGG |
| | | GTAAACATCTGGGGGGATCAAGAAAGCCTGATGGTGCAATTTACACA |
| | | GTGGGCTCTCCTATCGACTACGGTGTGATCGTGGATACAAAGGCCTA |
| | | CTCTGGAGGATATAATTTGCCTATTGGACAAGCCGATGAAATGGAAA |
| | | GATATGTGGAGGAAAACCAGACTCGCGATAAGCACCTGAACCCAAAT |
| | | GAATGGTGGAAAGTGTACCCTTCATCTGTTACCGAATTTAAATTTTT |
| | | GTTCGTTTCCGGGCATTTCAAGGGGAACTACAAGGCACAGCTGACGA |
| | | GACTGAATCACATCACGAACTGCGACGGCGCTGTACTGTCCGTGGAA |
| | | GAGCTTTTGATCGGGGGCGAAATGATTAAGGCCGGCACACTGACGCT |
| | | GGAGGAGGTGCGGCGAAAATTTAATAATGGCGAGATCAATTTTAGGA |
| | | GT |
| 116 | Left ZFP | GCCGCTATGGCTGAGAGGCCCTTCCAGTGTCGAATCTGCATGCAGAA |
| | (ZFP-L)(na) | CTTCAGTCAGTCCGGCAACCTGGCCCGCCACATCCGCACCCACACCG |
| | Not diversified | GCGAGAAGCCTTTTGCCTGTGACATTTGTGGGAGGAAATTTGCCCTG |
| | | AAGCAGAACCTGTGTATGCATACCAAGATACACACGGGCGAGAAGCC |
| | | CTTCCAGTGTCGAATCTGCATGCAGAAGTTTGCCTGGCAGTCCAACC |
| | | TGCAGAACCATACCAAGATACACACGGGCGAGAAGCCCTTCCAGTGT |
| | | CGAATCTGCATGCGTAACTTCAGTACCTCCGGCAACCTGACCCGCCA |
| | | CATCCGCACCCACACCGGCGAGAAGCCTTTTGCCTGTGACATTTGTG |
| | | GGAGGAAATTTGCCCGCCGCTCCCACCTGACCTCCCATACCAAGATA |
| | | CACCTGCGG |
| 117 | Left ZFP | GCAGCAATGGCCGAACGACCCTTCCAATGCAGAATATGTATGCAGAA |
| | (ZFP-L)(na) | TTTTTCTCAGAGCGGGAACCTGGCGAGGCACATAAGAACCCATACAG |
| | Codon | GAGAGAAGCCATTCGCATGCGATATTTGCGGTAGAAAATTTGCACTC |
| | diversified | AAACAAAATCTCTGTATGCACACTAAAATCCATACAGGTGAAAAGCC |
| | Version 1 | TTTTCAGTGCAGGATTTGTATGCAAAAATTTGCTTGGCAAAGTAACT |
| | | TGCAGAACCACACAAAGATACACACAGGAGAGAAACCCTTCCAATGC |
| | | CGAATCTGTATGCGCAACTTCAGTAGATCCGGAAATTTGACTAGACA |
| | | TATTAGGACCCACACCGGCGAGAAGCCATTTGCCTGCGATATTTGTG |
| | | GACGGAAATTCGCACGACGCAGCCATCTGACCAGTCATACTAAGATT |
| | | CATCTCCGC |
| 118 | Left ZFP | GCCGCCATGGCAGAGAGACCCTTTCAATGTAGAATCTGTATGCAAAA |
| | (ZFP-L)(na) | TTTCTCTCAGAGTGGTAACCTTGCAAGACACATCAGAACTCATACAG |
| | Codon | GTGAGAAGCCGTTTGCATGTGACATTTGCGGTAGGAAATTTGCCTTG |
| | diversified | AAACAGAATCTTTGTATGCACACAAAAATCCATACTGGTGAAAAGCC |
| | Version 2 | ATTCCAATGCCGCATCTGTATGCAAAAATTCGCGTGGCAGTCCAATT |
| | | TGCAGAACCATACCAAGATTCACACGGGAGAAAAACCATTTCAGTGC |
| | | CGCATCTGCATGCGCAACTTTTCTAGATCAGGAAACCTTAGACGACA |
| | | TATTCGGACGCACACTGGAGAAAAACCATTTGCTTGTGACATATGCG |
| | | GCCGAAAATTTGCCAGACGCTCTCATCTCACCTCACATACTAAGATT |
| | | CATTTGCGC |
| 119 | Left ZFP | GCCGCGATGGCAGAGAGACCATTTCAGTGTAGAATCTGTATGCAGAA |
| | (ZFP-L)(na) | CTTTTCCCAATCAGGAAACCTGGCACGACACATTAGAACCCATACTG |
| | Codon | GAGAAAAGCCGTTCGCTTGCGACATTTGCGGTAGAAAATTTGCTTTG |
| | diversified | AAACAGAACTTGTGTATGCATACCAAGATTCATACCGGCGAAAAACC |
| | Version 3 | ATTTCAATGCAGGATTTGTATGCAGAAGTTCGCCTGGCAATCCAATT |
| | | TGCAGAATCATACTAAAATTCATACCGGAGAAAAACCATTCCAATGC |
| | | CGCATTTGTATGAGAAACTTTTCTACCTCTGGCAATCTCACCAGACA |
| | | TATCAGAACACACACAGGCGAGAAACCGTTCGCATGCGATATCTGTG |
| | | GGCGAAAGTTTGCCAGAAGATCCCATCTCACATCACATACTAAAATA |
| | | CATTTGCGA |
| 120 | Left ZFP | GCAGCAATGGCCGAGAGACCTTTTCAGTGCAGGATTTGTATGCAAAA |
| | (ZFP-L)(na) | CTTCTCTCAGTCCGGTAACCTGGCCCGGCACATACGAACACATACCG |
| | Codon | GCGAAAAACCCTTTGCTTGCGACATCTGCGGAAGAAAGTTCGCTCTT |
| | diversified | AAACAGAACCTGTGCATGCATACAAAAATTCATACAGGTGAGAAGCC |
| | Version 4 | ATTCCAATGCAGAATATGTATGCAGAAATTCGCCTGGCAAAGCAACC |
| | | TGCAAAACCACACTAAGATCCACACAGGGGAAAAGCCTTTTCAATGT |
| | | AGAATCTGTATGAGAAACTTTAGTACATCCGGAAATCTCACACGACA |
| | | TATCAGAACCCACACTGGAGAAAAACCTTTTGCCTGCGACATCTGCG |
| | | GAAGAAAATTCGCCCGAAGGTCCCACTTGACTAGTCATACCAAAATC |
| | | CACTTGCGA |
| 121 | Left ZFP | GCAGCAATGGCAGAGAGACCATTTCAGTGCAGAATATGTATGCAAAA |
| | (ZFP-L)(na) | CTTCTCCCAGAGCGGTAATCTGGCTAGGCATATTAGAACACACACCG |
| | Codon | GGGAAAAACCTTTCGCTTGCGATATATGTGGTAGAAAGTTCGCCCTC |
| | diversified | AAACAGAATCTGTGCATGCACACTAAAATCCATACAGGAGAAAAGCC |
| | Version 5 | CTTTCAGTGTAGAATTTGTATGCAGAAATTTGCTTGGCAGTCAAATT |
| | | TGCAAAATCACACCAAAATACACACAGGAGAAAAACCATTTCAGTGT |
| | | AGAATATGTATGAGAAATTTTTCCACTTCCGGAAATCTGACCAGACA |
| | | TATACGGACACACACTGGGGAAAAGCCCTTCGCTTGCGACATCTGCG |
| | | GAAGAAAGTTCGCTAGACGGTCCCACTTGACATCCCACACTAAGATA |
| | | CATCTTCGC |
| 122 | Left ZFP | GCAGCCATGGCCGAACGCCCATTTCAATGTAGAATTTGTATGCAGAA |
| | (ZFP-L)(na) | TTTTTCACAATCAGGAAACCTGGCTAGACATATCAGAACACATACTG |
| | Codon | GAGAAAAGCCCTTTGCTTGTGATATCTGTGGAAGGAAATTCGCCCTG |
| | diversified | AAACAAAACCTCTGTATGCACACAAAGATCCACACCGGCGAAAAGCC |
| | Version 6 | TTTCCAGTGTAGGATATGCATGCAAAAATTCGCCTGGCAGTCCAATC |
| | | TGCAGAACCATACCAAAATTCATACTGGTGAAAAGCCATTTCAGTGC |
| | | AGAATATGTATGAGAAACTTTAGCACTTCAGGAAATCTCACAAGACA |
| | | TATAAGAACACATACAGGGGAAAAACCTTTTGCTTGCGATATCTGCG |
| | | GCAGGAAATTCGCTCGGAGAAGTCATCTCACAAGCCATACAAAAATC |
| | | CACCTGCGA |
| 123 | Right ZFP | GCCGCTATGGCTGAGAGGCCCTTCCAGTGTCGAATCTGCATGCGTAA |
| | (ZFP-L)(na) | CTTCAGTCAGTCCTCCGACCTGTCCCGCCACATCCGCACCCACACCG |
| | Not diversified | GCGAGAAGCCTTTTGCCTGTGACATTTGTGGGAGGAAATTTGCCCTG |
| | | AAGCACAACCTGCTGACCCATACCAAGATACACACGGGCGAGAAGCC |
| | | CTTCCAGTGTCGAATCTGCATGCAGAACTTCAGTGACCAGTCCAACC |
| | | TGCGCGCCCACATCCGCACCCACACCGGCGAGAAGCCTTTTGCCTGT |
| | | GACATTTGTGGGAGGAAATTTGCCCGCAACTTCTCCCTGACCATGCA |
| | | TACCAAGATACACACCGGAGAGCGCGGCTTCCAGTGTCGAATCTGCA |
| | | TGCGTAACTTCAGTCTGCGCCACGACCTGGAGCGCCACATCCGCACC |
| | | CACACCGGCGAGAAGCCTTTTGCCTGTGACATTTGTGGGAGGAAATT |
| | | TGCCCACCGCTCCAACCTGAACAAGCATACCAAGATACACCTGCGG |
| 124 | Right ZFP | GCTGCTATGGCTGAAAGACCTTTTCAATGTCGAATCTGCATGAGGAA |
| | (ZFP-L)(na) | TTTTAGTCAGTCATCCGACCTGAGCAGACACATTCGAACCCATACTG |
| | Codon | GTGAAAAGCCATTTGCTTGCGATATATGTGGGAGAAAATTTGCGTTG |
| | diversified | AAACACAATCTGCTGACCCATACCAAGATTCATACCGGAGAAAAACC |
| | Version 1 | ATTCCAATGCCGCATTTGTATGCAGAACTTTAGTGACCAGTCAAATC |
| | | TCCGCGCTCACATTCGAACCCACACTGGCGAAAAACCCTTTGCTTGT |
| | | GACATTTGCGGTCGGAAGTTTGCCCGAAATTTTTCTCTGACAATGCA |
| | | CACAAAAATCCACACCGGGGAACGCGGCTTTCAATGTAGGATCTGTA |
| | | TGAGAAATTTTAGCCTTAGACATGATTTGGAACGACATATCAGGACC |
| | | CATACAGGCGAGAAACCATTTGCGTGCGATATTTGTGGCAGGAAATT |
| | | CGCACATAGAAGTAATCTGAACAAGCATACAAAAATTCATCTCAGA |
| 125 | Right ZFP | GCTGCCATGGCCGAGAGACCATTTCAATGTCGGATTTGCATGCGCAA |
| | (ZFP-L)(na) | TTTTTCCCAGTCCTCTGACCTTAGCCGGCATATTCGGACACACACAG |
| | Codon | GTGAAAAACCCTTCGCATGCGACATTTGCGGAAGAAAATTCGCTCTG |
| | diversified | AAACACAACCTGCTTACCCATACAAAGATCCACACCGGCGAGAAACC |
| | Version 2 | GTTTCAATGCCGAATCTGTATGCAAAATTTTAGTGATCAAAGTAATC |
| | | TGAGAGCACATATTAGGACTCACACGGGCGAGAAGCCATTTGCGTGT |
| | | GATATCTGCGGCCGAAAATTCGCCCGGAATTTCTCTCTGACAATGCA |
| | | CACCAAAATCCACACTGGGGAACGAGGCTTTCAATGTAGAATATGTA |
| | | TGCGGAATTTCAGTCTGAGGCACGACCTGGAGCGGCACATCAGAACT |
| | | CACACCGGAGAAAAACCATTCGCTTGTGATATTTGCGGGAGGAAGTT |
| | | CGCCCATAGGAGCAATCTCAATAAACACACCAAAATACATCTTCGG |
| 126 | Right ZFP | GCCGCCATGGCCGAGCGCCCCTTCCAATGCCGCATATGCATGAGAAA |
| | (ZFP-L)(na) | TTTCAGCCAAAGTAGCGACCTGTCACGACACATTAGAACTCATACGG |
| | Codon | GGGAGAAGCCATTTGCTTGCGATATTTGTGGCAGAAAATTCGCACTC |
| | diversified | AAACACAACCTGCTCACACACACCAAGATACACACGGGAGAGAAGCC |
| | Version 3 | CTTCCAATGTAGAATATGTATGCAAAATTTCAGCGACCAAAGTAATT |
| | | TGAGAGCGCATATTCGAACTCACACCGGCGAAAAACCATTTGCCTGC |
| | | GATATTTGTGGGAGGAAATTTGCCAGGAATTTTTCACTCACCATGCA |
| | | CACTAAGATCCACACTGGCGAGCGCGGCTTCCAATGCAGAATCTGTA |
| | | TGCGAAACTTCAGTCTGCGGCATGACCTGGAAAGACATATAAGAACC |
| | | CACACCGGAGAAAAACCCTTTGCCTGCGACATATGTGGTAGAAAATT |
| | | CGCACATCGGAGTAACCTTAACAAACATACAAAGATCCACTTGAGA |
| 127 | Right ZFP | GCCGCTATGGCTGAAAGACCTTTCCAGTGTAGGATTTGCATGAGAAA |
| | (ZFP-L)(na) | TTTTTCCCAATCATCCGACCTTTCAAGGCATATTAGGACACACACCG |
| | Codon | GGGAAAAGCCATTTGCTTGTGATATCTGCGGGCGCAAATTTGCTCTT |
| | diversified | AAGCACAATCTTCTTACCCACACCAAAATTCATACAGGAGAAAAACC |
| | Version 4 | TTTTCAATGTAGAATCTGCATGCAAAACTTTTCCGATCAGTCAAATC |
| | | TTAGAGCTCATATCAGAACCCATACCGGGGAGAAACCCTTTGCCTGC |
| | | GACATATGCGGAAGAAAATTTGCTAGGAACTTTAGTCTGACCATGCA |
| | | TACCAAAATTCATACCGGCGAACGCGGTTTCCAGTGCAGGATTTGTA |
| | | TGAGAAATTTCTCACTGCGGCATGATCTTGAAAGACACATACGAACT |
| | | CATACCGGAGAAAAGCCATTCGCTTGCGATATTTGTGGTAGAAAATT |
| | | TGCCCACAGGTCTAACCTTAATAAGCACACCAAGATTCATCTCAGA |
| 128 | Right ZFP | GCAGCTATGGCCGAACGCCCTTTTCAATGCAGAATATGTATGCGAAA |
| | (ZFP-L)(na) | CTTCTCCCAAAGCTCTGATCTGTCAAGGCACATACGGACACACACCG |
| | Codon | GCGAAAAACCCTTTGCATGTGACATTTGTGGAAGAAAATTCGCACTT |
| | diversified | AAACACAATCTCCTGACTCATACAAAAATACATACAGGCGAAAAACC |
| | Version 5 | TTTCCAGTGCAGAATCTGTATGCAGAACTTTTCCGACCAATCCAATC |
| | | TTCGCGCCCACATTAGAACTCACACAGGGGAGAAACCTTTCGCTTGC |
| | | GACATATGCGGAAGAAAATTTGCCAGAAATTTTTCACTTACAATGCA |
| | | CACAAAAATACATACTGGGGAAAGAGGGTTTCAATGTCGAATCTGTA |
| | | TGAGAAATTTCAGTCTGCGCCATGATCTGGAGAGACATATAAGAACA |
| | | CACACAGGAGAGAAACCTTTTGCTTGTGACATATGCGGCCGAAAGTT |
| | | TGCTCATAGATCTAATCTTAACAAACATACAAAGATCCATCTTCGG |
| 129 | Right ZFP | GCTGCTATGGCTGAGAGACCTTTCCAATGTAGGATCTGTATGCGAAA |
| | (ZFP-L)(na) | CTTCTCCCAGAGCTCCGACCTGAGTCGCCATATAAGAACCCATACCG |
| | Codon | GAGAAAAACCATTTGCTTGTGACATTTGTGGCAGAAAGTTCGCTCTT |
| | diversified | AAACACAACCTGCTTACACATACTAAAATACACACAGGGGAGAAACC |
| | Version 6 | CTTTCAATGCCGGATCTGTATGCAAAACTTTAGCGATCAATCAAACT |
| | | TGCGAGCCCATATCCGCACTCACACCGGCGAGAAGCCTTTTGCATGC |
| | | GATATATGTGGACGGAAATTTGCTAGAAACTTCTCATTGACCATGCA |
| | | TACAAAAATACACACCGGGGAACGAGGATTTCAATGTCGAATTTGTA |
| | | TGAGAAATTTTAGCCTTAGGCACGACTTGGAACGGCACATAAGAACC |
| | | CACACCGGAGAGAAGCCTTTTGCTTGTGATATTTGCGGCAGAAAGTT |
| | | CGCCCATCGCAGCAATCTTAACAAGCACACCAAGATTCATTTGAGA |
| 136 | Left ZFP | AAMAERPFQCRICMQNFSQSGNLARHIRTHTGEKPFACDICGRKFAL |
| | (ZFP-L) | KQNLCMHTKIHTGEKPFQCRICMQKFAWQSNLQNHTKIHTGEKPFQC |
| | protein (aa) | RICMRNFSTSGNLTRHIRTHTGEKPFACDICGRKFARRSHLTSHTKI |
| | | HLR |
| 137 | Right ZFP | AAMAERPFQCRICMRNFSQSSDLSRHIRTHTGEKPFACDICGRKFAL |
| | (ZFP-R) | KHNLLTHTKIHTGEKPFQCRICMQNFSDQSNLRAHIRTHTGEKPFAC |
| | protein (aa) | DICGRKFARNFSLTMHTKIHTGERGFQCRICMRNFSLRHDLERHIRT |
| | | HTGEKPFACDICGRKFAHRSNLNKHTKIHLR |
| 138 | 2A peptide | GSGEGRGSLLTCGDVEENPGP |
| | (T2A) | |
| 139 | Left ZFN | CCCAAGAAGAAGAGGAAGGTCGGCATTCATGGGGTACCCGCCGCTAT |
| | with N-terminal | GGCTGAGAGGCCCTTCCAGTGTCGAATCTGCATGCAGAACTTCAGTC |
| | modifications | AGTCCGGCAACCTGGCCCGCCACATCCGCACCCACACCGGCGAGAAG |
| | (na) | CCTTTTGCCTGTGACATTTGTGGGAGGAAATTTGCCCTGAAGCAGAA |
| | (comprising | CCTGTGTATGCATACCAAGATACACACGGGCGAGAAGCCCTTCCAGT |
| | NLS, ZFP-L, | GTCGAATCTGCATGCAGAAGTTTGCCTGGCAGTCCAACCTGCAGAAC |
| | and FokI) | CATACCAAGATACACACGGGCGAGAAGCCCTTCCAGTGTCGAATCTG |
| | Not diversified | CATGCGTAACTTCAGTACCTCCGGCAACCTGACCCGCCACATCCGCA |
| | | CCCACACCGGCGAGAAGCCTTTTGCCTGTGACATTTGTGGGAGGAAA |
| | | TTTGCCCGCCGCTCCCACCTGACCTCCCATACCAAGATACACCTGCG |
| | | GGGATCCCAGCTGGTGAAGAGCGAGCTGGAGGAGAAGAAGTCCGAGC |
| | | TGCGGCACAAGCTGAAGTACGTGCCCCACGAGTACATCGAGCTGATC |
| | | GAGATCGCCAGGAACAGCACCCAGGACCGCATCCTGGAGATGAAGGT |
| | | GATGGAGTTCTTCATGAAGGTGTACGGCTACAGGGGAAAGCACCTGG |
| | | GCGGAAGCAGAAAGCCTGACGGCGCCATCTATACAGTGGGCAGCCCC |
| | | ATCGATTACGGCGTGATCGTGGACACAAAGGCCTACAGCGGCGGCTA |
| | | CAATCTGCCTATCGGCCAGGCCGACGAGATGGAGAGATACGTGGAGG |
| | | AGAACCAGACCCGGGATAAGCACCTCAACCCCAACGAGTGGTGGAAG |
| | | GTGTACCCTAGCAGCGTGACCGAGTTCAAGTTCCTGTTCGTGAGCGG |
| | | CCACTTCAAGGGCAACTACAAGGCCCAGCTGACCAGGCTGAACCACA |
| | | TCACCAACTGCGACGGCGCCGTGCTGAGCGTGGAGGAGCTGCTGATC |
| | | GGCGGCGAGATGATCAAAGCCGGCACCCTGACACTGGAGGAGGTGCG |
| | | GCGCAAGTTCAACAACGGCGAGATCAACTTCAGATCT |
| 140 | Left ZFN | CCAAAGAAGAAAAGAAAAGTGGGGATCCATGGTGTACCCGCAGCAAT |
| | with N-terminal | GGCCGAACGACCCTTCCAATGCAGAATATGTATGCAGAATTTTTCTC |
| | modifications | AGAGCGGGAACCTGGCGAGGCACATAAGAACCCATACAGGAGAGAAG |
| | (na) | CCATTCGCATGCGATATTTGCGGTAGAAAATTTGCACTCAAACAAAA |
| | (comprising | TCTCTGTATGCACACTAAAATCCATACAGGTGAAAAGCCTTTTCAGT |
| | NLS, ZFP-L, | GCAGGATTTGTATGCAAAAATTTGCTTGGCAAAGTAACTTGCAGAAC |
| | and FokI) | CACACAAAGATACACACAGGAGAGAAACCCTTCCAATGCCGAATCTG |
| | Codon | TATGCGCAACTTCAGTACATCCGGAAATTTGACTAGACATATTAGGA |
| | diversified | CCCACACCGGCGAGAAGCCATTTGCCTGCGATATTTGTGGACGGAAA |
| | Version 1 | TTCGCACGACGCAGCCATCTGACCAGTCATACTAAGATTCATCTCCG |
| | | CGGCAGCCAGCTTGTGAAGTCCGAACTGGAGGAAAAGAAGAGCGAAC |
| | | TGCGCCACAAATTGAAATACGTTCCGCATGAGTACATAGAGCTCATT |
| | | GAAATCGCTAGAAACTCTACCCAAGACAGGATACTGGAAATGAAAGT |
| | | GATGGAATTTTTCATGAAAGTTTATGGTTATAGGGGCAAACATCTGG |
| | | GTGGCTCTCGCAAGCCCGATGGGGCCATTTATACTGTCGGCTCACCT |
| | | ATCGACTATGGCGTCATTGTGGATACCAAGGCTTATTCTGGAGGATA |
| | | CAACCTGCCCATCGGACAAGCAGACGAAATGGAAAGATACGTCGAGG |
| | | AGAATCAAACCCGAGACAAGCATCTGAACCCAAACGAGTGGTGGAAA |
| | | GTGTACCCGAGCAGCGTTACTGAGTTCAAATTTCTCTTTGTAAGCGG |
| | | ACATTTTAAAGGGAATTACAAAGCACAACTGACTAGGCTGAACCATA |
| | | TAACCAACTGTGACGGGGCCGTATTGAGTGTGGAAGAGCTTCTGATT |
| | | GGAGGAGAGATGATTAAGGCTGGCACACTGACTCTCGAAGAAGTGAG |
| | | GCGCAAATTCAATAACGGTGAAATCAACTTCCGGTCT |
| 141 | Left ZFN | CCTAAAAAAAAGCGGAAAGTGGGAATTCACGGCGTGCCCGCCGCCAT |
| | with N-terminal | GGCAGAGAGACCCTTTCAATGTAGAATCTGTATGCAAAATTTCTCTC |
| | modifications | AGAGTGGTAACCTTGCAAGACACATCAGAACTCATACAGGTGAGAAG |
| | (na) | CCGTTTGCATGTGACATTTGCGGTAGGAAATTTGCCTTGAAACAGAA |
| | (comprising | TCTTTGTATGCACACAAAAATCCATACTGGTGAAAAGCCATTCCAAT |
| | NLS, ZFP-L, | GCCGCATCTGTATGCAAAAATTCGCGTGGCAGTCCAATTTGCAGAAC |
| | and FokI) | CATACCAAGATTCACACGGGAGAAAAACCATTTCAGTGCCGCATCTG |
| | Codon | CATGCGCAACTTTTCTACATCAGGAAACCTTACACGACATATTCGGA |
| | diversified | CGCACACTGGAGAAAAACCATTTGCTTGTGACATATGCGGCCGAAAA |
| | Version 2 | TTTGCCAGACGCTCTCATCTCACCTCACATACTAAGATTCATTTGCG |
| | | CGGAAGTCAGCTGGTGAAGAGTGAATTGGAAGAAAAAAAGTCAGAGC |
| | | TGAGACACAAACTGAAATATGTTCCACACGAGTAGATCGAGCTTATC |
| | | GAGATAGCAAGAAACTCCACCCAGGACAGAATTTTGGAAATGAAAGT |
| | | TATGGAATTCTTTATGAAAGTGTATGGCTACAGGGGTAAACATCTGG |
| | | GGGGATCAAGAAAGCCTGATGGTGCAATTTACACAGTGGGCTCTCCT |
| | | ATCGACTACGGTGTGATCGTGGATACAAAGGCCTACTCTGGAGGATA |
| | | TAATTTGCCTATTGGACAAGCCGATGAAATGGAAAGATATGTGGAGG |
| | | AAAACCAGACTCGCGATAAGCACCTGAACCCAAATGAATGGTGGAAA |
| | | GTGTACCCTTCATCTGTTACCGAATTTAAATTTTTGTTCGTTTCCGG |
| | | GCATTTCAAGGGGAACTACAAGGCACAGCTGACGAGACTGAATCACA |
| | | TCACGAACTGCGACGGCGCTGTACTGTCCGTGGAAGAGCTTTTGATC |
| | | GGGGGCGAAATGATTAAGGCCGGCACACTGACGCTGGAGGAGGTGCG |
| | | GCGAAAATTTAATAATGGCGAGATCAATTTTAGGAGT |
| 142 | Left ZFN | CCCAAAAAGAAGAGAAAAGTGGGAATCCACGGTGTACCGGCCGCGAT |
| | with N- | GGCAGAGAGACCATTTCAGTGTAGAATCTGTATGCAGAACTTTTCCC |
| | terminal | AATCAGGAAACCTGGCACGACACATTAGAACCCATACTGGAGAAAAG |
| | modifications | CCGTTCGCTTGCGACATTTGCGGTAGAAAATTTGCTTTGAAACAGAA |
| | (na) | CTTGTGTATGCATACCAAGATTCATACCGGCGAAAAACCATTTCAAT |
| | (comprising | GCAGGATTTGTATGCAGAAGTTCGCCTGGCAATCCAATTTGCAGAAT |
| | NLS, ZFP-L, | CATACTAAAATTCATACCGGAGAAAAACCATTCCAATGCCGCATTTG |
| | and FokI) | TATGAGAAACTTTTCTACCTCTGGCAATCTCACCAGACATATCAGAA |
| | Codon | CACACACAGGCGAGAAACCGTTCGCATGCGATATCTGTGGGCGAAAG |
| | diversified | TTTGCCAGAAGATCCCATCTCACATCACATACTAAAATACATTTGCG |
| | Version 3 | AGGAAGTCAACTGGTCAAGTCCGAACTGGAGGAAAAAAAAAGTGAGC |
| | | TGCGACACAAGTTGAAGTACGTACCACACGAATACATCGAGCTGATT |
| | | GAGATAGCACGGAACTCTACCCAGGATAGAATACTGGAGATGAAAGT |
| | | TATGGAATTCTTTATGAAGGTGTACGGATACAGGGGGAAGCATCTTG |
| | | GCGGGAGCCGGAAACCAGACGGAGCAATCTATACCGTCGGGTCACCT |
| | | ATAGACTATGGAGTTATTGTCGATACAAAGGCCTATTCAGGAGGTTA |
| | | TAATCTGCCAATCGGCCAAGCCGACGAGATGGAGAGGTACGTGGAGG |
| | | AAAATCAGACCAGAGACAAGCACCTGAACCCTAATGAATGGTGGAAA |
| | | GTGTACCCTAGCAGCGTCACTGAGTTCAAATTCCTGTTCGTCAGCGG |
| | | TCATTTTAAAGGAAATTATAAAGCCCAGCTCACTAGACTCAACCATA |
| | | TTACAAACTGCGACGGAGCCGTACTTAGCGTTGAAGAGTTGCTTATC |
| | | GGAGGAGAGATGATCAAAGCCGGAACCCTCACACTTGAAGAAGTGCG |
| | | AAGAAAATTCAATAACGGAGAGATAAATTTTAGGAGT |
| 143 | Left ZFN | CCTAAGAAGAAGAGAAAAGTTGGAATACATGGAGTCCCCGCAGCAAT |
| | with N-terminal | GGCCGAGAGACCTTTTCAGTGCAGGATTTGTATGCAAAACTTCTCTC |
| | modifications | AGTCCGGTAACCTGGCCCGGCACATACGAACACATACCGGCGAAAAA |
| | (na) | CCCTTTGCTTGCGACATCTGCGGAAGAAAGTTCGCTCTTAAACAGAA |
| | (comprising | CCTGTGCATGCATACAAAAATTCATACAGGTGAGAAGCCATTCCAAT |
| | NLS, ZFP-L, | GCAGAATATGTATGCAGAAATTCGCCTGGCAAAGCAACCTGCAAAAC |
| | and FokI) | CACACTAAGATCCACACAGGGGAAAAGCCTTTTCAATGTAGAATCTG |
| | Codon | TATGAGAAACTTTAGTACATCCGGAAATCTCACACGACATATCAGAA |
| | diversified | CCCACACTGGAGAAAAACCTTTTGCCTGCGACATCTGCGGAAGAAAA |
| | Version 4 | TTCGCCCGAAGGTCCCACTTGACTAGTCATACCAAAATCCACTTGCG |
| | | AGGCTCACAGCTGGTTAAATCCGAACTTGAAGAAAAAAAAAGTGAAC |
| | | TGCGGCATAAACTGAAGTATGTCCCCCATGAATATATCGAACTGATA |
| | | GAAATCGCCCGAAATAGCACCCAAGATAGAATCCTCGAAATGAAGGT |
| | | TATGGAATTTTTCATGAAGGTCTATGGATATAGGGGCAAGCACCTTG |
| | | GCGGATCCCGGAAACCTGATGGAGCTATCTACACAGTGGGCTCACCA |
| | | ATAGACTATGGAGTTATCGTCGATACAAAAGCATACAGCGGAGGATA |
| | | CAATTTGCCAATAGGTCAAGCAGATGAGATGGAAAGATACGTGGAGG |
| | | AAAACCAAACAAGAGATAAGCATCTGAACCCCAACGAATGGTGGAAA |
| | | GTGTACCCCAGTTCTGTAACCGAATTTAAGTTCTTGTTCGTTTCAGG |
| | | TCACTTCAAGGGTAATTACAAGGCTCAACTGACTAGACTCAACCATA |
| | | TTACAAATTGCGATGGTGCTGTGCTTTCCGTGGAAGAATTGCTGATT |
| | | GGTGGAGAGATGATAAAAGCTGGTACCCTCACCTTGGAAGAAGTGCG |
| | | CAGAAAATTCAATAATGGCGAGATCAACTTCCGAAGT |
| 144 | Left ZFN | CCCAAGAAGAAACGAAAAGTAGGAATCCATGGCGTGCCTGCAGCAAT |
| | with N- | GGCAGAGAGACCATTTCAGTGCAGAATATGTATGCAAAACTTCTCCC |
| | terminal | AGAGCGGTAATCTGGCTAGGCATATTAGAACACACACCGGGGAAAAA |
| | modifications | CCTTTCGCTTGCGATATATGTGGTAGAAAGTTCGCCCTCAAACAGAA |
| | (na) | TCTGTGCATGCACACTAAAATCCATACAGGAGAAAAGCCCTTTCAGT |
| | (comprising | GTAGAATTTGTATGCAGAAATTTGCTTGGCAGTCAAATTTGCAAAAT |
| | NLS, ZFP-L, | CACACCAAAATACACACAGGAGAAAAACCATTTCAGTGTAGAATATG |
| | and FokI) | TATGAGAAATTTTTCCACTTCCGGAAATCTGAGCAGACATATACGGA |
| | Codon | CACACACTGGGGAAAAGCCCTTCGCTTGCGACATCTGCGGAAGAAAG |
| | diversified | TTCGCTAGACGGTCCCACTTGACATCCCACACTAAGATACATCTTCG |
| | Version 5 | CGGTAGCCAACTGGTGAAAAGTGAACTGGAGGAAAAAAAATCTGAGC |
| | | TGAGACATAAACTGAAATACGTACCACATGAATACATAGAACTTATA |
| | | GAAATAGCTAGGAACTCCACCCAGGACAGAATACTTGAAATGAAGGT |
| | | CATGGAGTTTTTTATGAAAGTTTACGGATACAGGGGCAAACACCTTG |
| | | GAGGGTCTCGGAAGCCTGATGGCGCAATTTATACCGTGGGTAGCCCT |
| | | ATAGATTATGGAGTGATTGTGGATACAAAGGCTTACAGTGGCGGCTA |
| | | TAATTTGCCTATCGGACAGGCCGATGAGATGGAAAGATACGTTGAAG |
| | | AAAACCAAACACGAGATAAGCATCTGAACCCCAATGAATGGTGGAAA |
| | | GTGTATCCTTCAAGCGTTACCGAGTTTAAGTTCCTCTTCGTTTCTGG |
| | | GCATTTCAAGGGCAACTACAAAGCTCAGCTTACAAGACTCAACCACA |
| | | TAACCAATTGTGATGGAGCAGTCCTCAGCGTGGAAGAACTCCTTATT |
| | | GGGGGTGAGATGATTAAAGCAGGGACCCTTACTCTTGAAGAGGTTAG |
| | | AAGAAAATTCAATAACGGAGAGATTAATTTTAGAAGT |
| 145 | Left ZFN | CCTAAGAAGAAAAGAAAGGTCGGCATTCATGGTGTGCCTGCAGCCAT |
| | with N-terminal | GGCCGAACGCCCATTTCAATGTAGAATTTGTATGCAGAATTTTTCAC |
| | modifications | AATCAGGAAACCTGGCTAGACATATCAGAACACATACTGGAGAAAAG |
| | (na) | CCCTTTGCTTGTGATATCTGTGGAAGGAAATTCGCCCTGAAACAAAA |
| | (comprising | CCTCTGTATGCACACAAAGATCCACACCGGCGAAAAGCCTTTCCAGT |
| | NLS, ZFP-L, | GTAGGATATGCATGCAAAAATTCGCCTGGCAGTCCAATCTGCAGAAC |
| | and FokI) | CATACCAAAATTCATACTGGTGAAAAGCCATTTCAGTGCAGAATATG |
| | Codon | TATGAGAAACTTTAGCACTTCAGGAAATCTCACAAGACATATAAGAA |
| | diversified | CACATACAGGGGAAAAACCTTTTGCTTGCGATATCTGCGGCAGGAAA |
| | Version 6 | TTCGCTCGGAGAAGTCATCTCACAAGCCATACAAAAATCCACCTGCG |
| | | AGGAAGCCAGCTGGTCAAGTCTGAACTGGAAGAAAAAAAAAGCGAAC |
| | | TGCGGCATAAACTCAAATACGTCCCACATGAATACATTGAGCTCATC |
| | | GAAATTGCTAGAAACTCTAGTCAAGATAGGATATTGGAGATGAAGGT |
| | | AATGGAATTCTTCATGAAGGTTTATGGATATAGAGGAAAACATCTTG |
| | | GAGGCAGTAGGAAACCCGATGGCGCTATCTACACCGTAGGGAGTCCA |
| | | ATCGACTACGGCGTGATTGTTGACACCAAAGCCTATTCTGGAGGGTA |
| | | TAATCTCCCAATTGGACAGGCAGATGAGATGGAAAGATATGTAGAAG |
| | | AAAATCAGACAAGAGATAAGCACCTTAACCCTAACGAGTGGTGGAAA |
| | | GTGTACCCAAGCAGTGTTACTGAATTTAAATTTCTTTTTGTATCAGG |
| | | ACACTTTAAAGGCAATTACAAAGCACAACTGACCAGACTCAATCACA |
| | | TTACCAATTGCGACGGAGCCGTACTGAGCGTGGAGGAGTTGCTGATC |
| | | GGAGGCGAAATGATTAAAGCTGGCACTCTGACCCTGGAAGAAGTAAG |
| | | AAGAAAGTTCAATAATGGAGAAATAAACTTTCGCTCC |
| 146 | Right ZFN | CCCAAGAAGAAGAGGAAGGTCGGCATTCATGGGGTACCCGCCGCTAT |
| | with N-terminal | GGCTGAGAGGCCCTTCCAGTGTCGAATCTGCATGCGTAACTTCAGTC |
| | modifications | AGTCCTCCGACCTGTCCCGCCACATCCGCACCCACACCGGCGAGAAG |
| | (na) | CCTTTTGCCTGTGACATTTGTGGGAGGAAATTTGCCCTGAAGCACAA |
| | (comprising | CCTGCTGACCCATACCAAGATACACACGGGCGAGAAGCCCTTCCAGT |
| | NLS, ZFP-R, | GTCGAATCTGCATGCAGAACTTCAGTGACCAGTCCAACCTGCGCGCC |
| | and FokI) | CACATCCGCACCCACACCGGCGAGAAGCCTTTTGCCTGTGACATTTG |
| | Not diversified | TGGGAGGAAATTTGCCCGCAACTTCTCCCTGACCATGCATACCAAGA |
| | | TACACACCGGAGAGCGCGGCTTCCAGTGTCGAATCTGCATGCGTAAC |
| | | TTCAGTCTGCGCCACGACCTGGAGCGCCACATCCGCACCCACACCGG |
| | | CGAGAAGCCTTTTGCCTGTGACATTTGTGGGAGGAAATTTGCCCACC |
| | | GCTCCAACCTGAACAAGCATACCAAGATACACCTGCGGGGATCCCAG |
| | | CTGGTGAAGAGCGAGCTGGAGGAGAAGAAGTCCGAGCTGCGGCACAA |
| | | GCTGAAGTACGTGCCCCACGAGTACATCGAGCTGATCGAGATCGCCA |
| | | GGAACAGCACCCAGGACCGCATCCTGGAGATGAAGGTGATGGAGTTC |
| | | TTCATGAAGGTGTACGGCTACAGGGGAAAGCACCTGGGCGGAAGCAG |
| | | AAAGCCTGACGGCGCCATCTATACAGTGGGCAGCCCCATCGATTACG |
| | | GCGTGATCGTGGACACAAAGGCCTACAGCGGCGGCTACAATCTGAGC |
| | | ATCGGCCAGGCCGACGAGATGCAGAGATACGTGAAGGAGAACCAGAC |
| | | CCGGAATAAGCACATCAACCCCAACGAGTGGTGGAAGGTGTACCCTA |
| | | GCAGCGTGACCGAGTTCAAGTTCCTGTTCGTGAGCGGCCACTTCAAG |
| | | GGCAACTACAAGGCCCAGCTGACCAGGCTGAACCGCAAAACCAACTG |
| | | CAATGGCGCCGTGCTGAGCGTGGAGGAGCTGCTGATCGGCGGCGAGA |
| | | TGATCAAAGCCGGCACCCTGACACTGGAGGAGGTGCGGCGCAAGTTC |
| | | AACAACGGCGAGATCAACTTC |
| 147 | Right ZFN | CCTAAAAAGAAACGAAAAGTGGGCATTCACGGCGTACCTGCTGCTAT |
| | with N-terminal | GGCTGAAAGACCTTTTCAATGTCGAATCTGCATGAGGAATTTTAGTC |
| | modifications | AGTCATCCGACCTGAGCAGACACATTCGAACCCATACTGGTGAAAAG |
| | (na) | CCATTTGCTTGCGATATATGTGGGAGAAAATTTGCGTTGAAACACAA |
| | (comprising | TCTGCTGACCCATACCAAGATTCATACCGGAGAAAAACCATTCCAAT |
| | NLS, ZFP-R, | GCCGCATTTGTATGCAGAACTTTAGTGACCAGTCAAATCTCCGCGCT |
| | and FokI) | CACATTCGAACCCACACTGGCGAAAAACCCTTTGCTTGTGACATTTG |
| | Codon | CGGTCGGAAGTTTGCCCGAAATTTTTCTCTGACAATGCACACAAAAA |
| | diversified | TCCACACCGGGGAACGCGGCTTTCAATGTAGGATCTGTATGAGAAAT |
| | Version 1 | TTTAGCCTTAGACATGATTTGGAACGACATATCAGGACCCATACAGG |
| | | CGAGAAACCATTTGCGTGCGATATTTGTGGCAGGAAATTCGCACATA |
| | | GAAGTAATCTGAACAAGCATACAAAAATTCATCTCAGAGGAAGTCAG |
| | | CTGGTCAAAAGTGAACTGGAGGAAAAAAAGAGCGAACTGAGACACAA |
| | | ACTGAAGTACGTGCCACACGAATATATTGAGCTGATTGAGATCGCGA |
| | | GGAACTCAACACAGGACCGCATTCTGGAGATGAAAGTGATGGAGTTT |
| | | TTCATGAAAGTATATGGATATAGAGGAAAACACCTTGGGGGTAGCCG |
| | | AAAGCCGGACGGGGCGATCTACACTGTGGGGTCACCAATTGATTATG |
| | | GCGTAATTGTCGATACCAAAGCCTACAGTGGGGGGTACAATCTGAGT |
| | | ATAGGACAGGCTGATGAAATGCAACGATACGTTAAGGAGAATCAGAC |
| | | TAGGAATAAACATATCAATCCAAATGAATGGTGGAAAGTCTATCCCA |
| | | GCAGCGTGACAGAATTTAAATTTTTGTTTGTCAGTGGACACTTCAAG |
| | | GGAAATTATAAGGCCCAGCTGACTAGACTGAATAGGAAAACCAATTG |
| | | TAATGGCGCAGTGCTTTCAGTGGAGGAACTGCTCATTGGAGGTGAGA |
| | | TGATCAAGGCTGGAACCCTGACGCTGGAGGAGGTGCGGAGGAAGTTT |
| | | AACAATGGAGAAATTAACTTT |
| 148 | Right ZFN | CCTAAGAAAAAGAGAAAAGTCGGAATCCACGGTGTCCCAGCTGCCAT |
| | with N-terminal | GGCCGAGAGACCATTTCAATGTCGGATTTGCATGCGCAATTTTTCCC |
| | modifications | AGTCCTCTGACCTTAGCCGGCATATTCGGACACACACAGGTGAAAAA |
| | (na) | CCCTTCGCATGCGACATTTGCGGAAGAAAATTCGCTCTGAAACACAA |
| | (comprising | CCTGCTTACCCATACAAAGATCCACACCGGCGAGAAACCGTTTCAAT |
| | NLS, ZFP-R, | GCCGAATCTGTATGCAAAATTTTAGTGATCAAAGTAATCTGAGAGCA |
| | and FokI) | CATATTAGGACTCACACGGGCGAGAAGCCATTTGCGTGTGATATCTG |
| | Codon | CGGCCGAAAATTCGCCCGGAATTTCTCTCTGACAATGCACACCAAAA |
| | diversified | TCCACACTGGGGAACGAGGCTTTCAATGTAGAATATGTATGCGGAAT |
| | Version 2 | TTCAGTCTGAGGCACGACCTGGAGCGGCACATCAGAACTCACACCGG |
| | | AGAAAAACCATTCGCTTGTGATATTTGCGGGAGGAAGTTCGCCCATA |
| | | GGAGCAATCTCAATAAACACACCAAAATACATCTTCGGGGTTCTCAA |
| | | CTGGTGAAATCCGAACTGGAAGAAAAGAAATCAGAATTGCGGCATAA |
| | | ACTGAAGTATGTGCCCCATGAGTACATAGAACTGATCGAGATCGCAA |
| | | GGAACTCTACCCAGGACAGAATACTTGAAATGAAGGTCATGGAATTT |
| | | TTTATGAAAGTGTACGGCTACAGAGGAAAACATTTGGGAGGCAGTCG |
| | | AAAACCAGATGGCGCAATCTATACAGTCGGGTCCCCCATAGATTACG |
| | | GAGTGATTGTCGACACAAAAGCCTATTCCGGAGGATATAACCTTAGT |
| | | ATCGGCCAGGCCGACGAGATGCAACGCTATGTGAAAGAAAACCAAAC |
| | | AAGAAATAAACATATCAATCCAAACGAGTGGTGGAAGGTATATCCAA |
| | | GCAGTGTCACAGAATTCAAATTCCTCTTCGTGAGTGGGCACTTTAAA |
| | | GGCAACTACAAAGCTCAATTGACCAGGCTCAATCGGAAAACTAATTG |
| | | CAATGGCGCAGTCCTTAGCGTCGAAGAATTGCTGATTGGCGGGGAAA |
| | | TGATTAAAGCAGGAACTTTGACCTTGGAGGAAGTAGGGAGAAAGTTT |
| | | AACAACGGCGAGATTAATTTT |
| 149 | Right ZFN | CCCAAGAAGAAAAGAAAAGTAGGAATTCACGGAGTCCCTGCCGCCAT |
| | with N-terminal | GGCCGAGCGCCCCTTCCAATGCCGCATATGCATGAGAAATTTCAGCC |
| | modifications | AAAGTAGCGACCTGTCACGACACATTAGAACTCATACGGGGGAGAAG |
| | (na) | CCATTTGCTTGCGATATTTGTGGCAGAAAATTCGCACTCAAACACAA |
| | (comprising | CCTGCTCACACACACCAAGATACACACGGGAGAGAAGCCCTTCCAAT |
| | NLS, ZFP-R, | GTAGAATATGTATGCAAAATTTCAGCGACCAAAGTAATTTGAGAGCG |
| | and FokI) | CATATTCGAACTCACACCGGCGAAAAACCATTTGCCTGCGATATTTG |
| | Codon | TGGGAGGAAATTTGCCAGGAATTTTTCACTCACCATGCACACTAAGA |
| | diversified | TCCACACTGGCGAGCGCGGCTTCCAATGCAGAATCTGTATGCGAAAC |
| | Version 3 | TTCAGTCTGCGGCATGACCTGGAAAGACATATAAGAACCCACACCGG |
| | | AGAAAAACCCTTTGCCTGCGACATATGTGGTAGAAAATTCGCACATC |
| | | GGAGTAACCTTAACAAACATACAAAGATCCACTTGAGAGGCAGTCAG |
| | | CTGGTGAAATCTGAGCTGGAAGAGAAGAAATCTGAACTGCGACATAA |
| | | ATTGAAGTACGTCCCACACGAGTAGATCGAGTTGATCGAAATTGCCC |
| | | GGAATAGCACCCAGGATAGAATATTGGAAATGAAAGTAATGGAGTTT |
| | | TTTATGAAGGTTTATGGTTACAGAGGCAAGCACCTTGGAGGAAGCAG |
| | | GAAACCAGATGGGGCGATTTACACCGTTGGGAGTCCCATCGATTACG |
| | | GAGTCATCGTGGACACAAAGGCCTATTCCGGAGGCTACAACCTCAGT |
| | | ATCGGGCAAGCCGATGAGATGCAGAGATATGTTAAAGAAAATCAGAC |
| | | GCGAAACAAGCACATTAACCCAAACGAATGGTGGAAAGTTTACCCTA |
| | | GCTCAGTGACAGAATTTAAGTTTCTGTTTGTCAGCGGCCACTTCAAG |
| | | GGGAATTATAAAGCACAACTGACCCGCCTGAACCGAAAAACCAACTG |
| | | TAACGGTGCTGTGCTGAGTGTCGAAGAGTTGCTTATCGGAGGAGAGA |
| | | TGATAAAGGCCGGCACACTGACGCTTGAAGAGGTACGGCGAAAATTC |
| | | AATAACGGAGAGATTAATTTT |
| 150 | Right ZFN | CCAAAAAAAAAACGCAAGGTTGGAATACACGGTGTACCTGCCGCTAT |
| | with N-terminal | GGCTGAAAGACCTTTCCAGTGTAGGATTTGCATGAGAAATTTTTCCC |
| | modifications | AATCATCCGACCTTTCAAGGCATATTAGGACACACACCGGGGAAAAG |
| | (na) | CCATTTGCTTGTGATATCTGCGGGCGCAAATTTGCTCTTAAGCACAA |
| | (comprising | TCTTCTTACCCACACCAAAATTCATACAGGAGAAAAACCTTTTCAAT |
| | NLS, ZFP-R, | GTAGAATCTGCATGCAAAACTTTTCCGATCAGTCAAATCTTAGAGCT |
| | and FokI) | CATATCAGAACCCATACCGGGGAGAAACCCTTTGCCTGCGACATATG |
| | Codon | CGGAAGAAAATTTGCTAGGAACTTTAGTCTGACCATGCATACCAAAA |
| | diversified | TTCATACCGGCGAACGCGGTTTCCAGTGCAGGATTTGTATGAGAAAT |
| | Version 4 | TTCTCACTGCGGCATGATCTTGAAAGACACATACGAACTCATACCGG |
| | | AGAAAAGCCATTCGCTTGCGATATTTGTGGTAGAAAATTTGCCCACA |
| | | GGTCTAACCTTAATAAGCACACCAAGATTCATCTCAGAGGATCTCAG |
| | | CTGGTCAAATCAGAACTTGAAGAGAAAAAAAGCGAACTGAGACATAA |
| | | ACTGAAGTACGTGCCTCATGAATACATAGAGCTCATTGAAATAGCTA |
| | | GGAATAGTACACAGGACAGGATACTTGAAATGAAGGTAATGGAATTT |
| | | TTCATGAAGGTTTATGGATACCGGGGGAAACATCTCGGGGGCAGCAG |
| | | AAAACCAGACGGAGCAATTTATACTGTCGGGAGTCCTATAGATTATG |
| | | GCGTTATCGTCGATACAAAGGCCTATTCCGGTGGGTACAACCTCTCA |
| | | ATTGGTCAGGCTGATGAGATGCAAAGATACGTCAAAGAAAACCAAAC |
| | | CAGAAATAAACATATAAATCCCAATGAATGGTGGAAAGTATACCCAA |
| | | GTTCCGTGACTGAATTCAAGTTCCTTTTCGTGTCTGGCCACTTTAAA |
| | | GGAAATTATAAAGCACAATTGACTAGACTGAATAGAAAAACAAACTG |
| | | TAACGGCGCAGTGCTGTCAGTGGAAGAACTGCTCATAGGTGGAGAGA |
| | | TGATCAAGGCCGGGACACTTACTCTTGAGGAAGTTAGAAGGAAGTTC |
| | | AACAACGGCGAAATCAACTTT |
| 151 | Right ZFN | CCAAAGAAAAAGAGGAAGGTGGGAATACATGGAGTACCAGCAGCTAT |
| | with N-terminal | GGCCGAACGCCCTTTTCAATGCAGAATATGTATGCGAAACTTCTCCC |
| | modifications | AAAGCTCTGATCTGTCAAGGCACATACGGACACACACCGGCGAAAAA |
| | (na) | CCCTTTGCATGTGACATTTGTGGAAGAAAATTCGCACTTAAACACAA |
| | (comprising | TCTCCTGACTCATACAAAAATACATACAGGCGAAAAACCTTTCCAGT |
| | NLS, ZFP-R, | GCAGAATCTGTATGCAGAACTTTTCCGACCAATCCAATCTTCGCGCC |
| | and FokI) | CACATTAGAACTCACACAGGGGAGAAACCTTTCGCTTGCGACATATG |
| | Codon | CGGAAGAAAATTTGCCAGAAATTTTTCACTTAGAATGCACACAAAAA |
| | diversified | TACATACTGGGGAAAGAGGGTTTCAATGTCGAATCTGTATGAGAAAT |
| | Version 5 | TTCAGTCTGCGCCATGATCTGGAGAGACATATAAGAACACACACAGG |
| | | AGAGAAACCTTTTGCTTGTGACATATGCGGCCGAAAGTTTGCTCATA |
| | | GATCTAATCTTAACAAACATACAAAGATCCATCTTCGGGGTTCACAA |
| | | CTGGTCAAGTCAGAATTGGAAGAGAAAAAATCTGAGCTGAGGCACAA |
| | | ATTGAAATACGTTCCTCACGAGTATATTGAACTTATCGAGATAGCCC |
| | | GCAATAGTACACAAGATAGAATCTTGGAGATGAAAGTTATGGAATTC |
| | | TTTATGAAAGTCTATGGCTATAGGGGAAAACACCTGGGGGGTAGCAG |
| | | GAAACCTGATGGAGCTATCTATACCGTAGGATCACCTATTGATTATG |
| | | GAGTAATTGTGGACACTAAGGCATATTCCGGAGGATATAATTTGAGT |
| | | ATTGGTCAGGCCGACGAAATGCAACGATACGTGAAGGAAAATCAGAC |
| | | CCGCAACAAACACATTAATCCCAATGAATGGTGGAAGGTATACCCTA |
| | | GTAGCGTTACAGAGTTTAAATTCCTTTTCGTCAGCGGCCACTTTAAA |
| | | GGAAATTATAAAGCACAACTCACCAGACTTAATCGAAAAACTAACTG |
| | | TAACGGCGCCGTACTGTCAGTGGAGGAGCTGCTCATTGGAGGCGAGA |
| | | TGATCAAGGCCGGTACTCTCACACTGGAAGAAGTTAGAAGAAAGTTC |
| | | AACAACGGGGAAATTAATTTC |
| 152 | Right ZFN | CCCAAAAAGAAAAGAAAGGTGGGTATTCACGGAGTTCCCGCTGCTAT |
| | with N-terminal | GGCTGAGAGACCTTTCCAATGTAGGATCTGTATGCGAAACTTCTCCC |
| | modifications | AGAGCTCCGACCTGAGTCGCCATATAAGAACCCATACCGGAGAAAAA |
| | (na) | CCATTTGCTTGTGACATTTGTGGCAGAAAGTTCGCTCTTAAACACAA |
| | (comprising | CCTGCTTACACATACTAAAATACACACAGGGGAGAAACCCTTTCAAT |
| | NLS, ZFP-R, | GCCGGATCTGTATGCAAAACTTTAGCGATCAATCAAACTTGCGAGCC |
| | and FokI) | CATATCCGCACTCACACCGGCGAGAAGCCTTTTGCATGCGATATATG |
| | Codon | TGGACGGAAATTTGCTAGAAACTTCTCATTGACCATGCATACAAAAA |
| | diversified | TAGACACCGGGGAACGAGGATTTCAATGTCGAATTTGTATGAGAAAT |
| | Version 6 | TTTAGCCTTAGGCACGACTTGGAACGGCACATAAGAACCCACACCGG |
| | | AGAGAAGCCTTTTGCTTGTGATATTTGCGGCAGAAAGTTCGCCCATC |
| | | GCAGCAATCTTAACAAGCACACCAAGATTCATTTGAGAGGTTCCCAG |
| | | CTGGTCAAAAGCGAACTTGAAGAAAAGAAATCCGAGCTTAGACACAA |
| | | ACTGAAATACGTGCCTCACGAGTATATTGAGCTGATTGAAATAGCAA |
| | | GGAATTCAACACAAGACAGGATCCTCGAAATGAAGGTTATGGAGTTT |
| | | TTCATGAAAGTTTACGGCTACAGAGGGAAGCATCTGGGCGGATCAAG |
| | | AAAACCAGACGGCGCAATCTACACAGTTGGATCCCCAATAGATTACG |
| | | GAGTGATTGTTGACACCAAGGCTTATTCAGGAGGTTACAATCTGTCC |
| | | ATTGGTCAGGCCGATGAAATGCAAAGATATGTTAAGGAAAATCAAAC |
| | | TCGAAACAAACACATTAATCCAAACGAATGGTGGAAAGTATATCCAA |
| | | GCTCCGTCACTGAATTTAAATTTTTGTTTGTATCCGGACATTTTAAG |
| | | GGCAACTATAAGGCTCAACTGACCAGACTGAATAGGAAGACCAATTG |
| | | TAACGGAGCTGTACTCAGCGTGGAAGAACTGCTTATTGGAGGCGAAA |
| | | TGATTAAGGCTGGCACACTTACACTCGAAGAAGTTAGAAGAAAATTC |
| | | AACAATGGTGAGATAAACTTC |
| 157 | Fok1 | CAGCTGGTGAAGAGCGAGCTGGAGGAGAAGAAGTCCGAGCTGCGGCA |
| | (Right ZFN) | CAAGCTGAAGTACGTGCCCCACGAGTACATCGAGCTGATCGAGATCG |
| | Not diversified | CCAGGAACAGCACCCAGGACCGCATCCTGGAGATGAAGGTGATGGAG |
| | (na) | TTCTTCATGAAGGTGTACGGCTACAGGGGAAAGCACCTGGGCGGAAG |
| | | CAGAAAGCCTGACGGCGCCATCTATACAGTGGGCAGCCCCATCGATT |
| | | ACGGCGTGATCGTGGACACAAAGGCCTACAGCGGCGGCTACAATCTG |
| | | AGCATCGGCCAGGCCGACGAGATGCAGAGATACGTGAAGGAGAACCA |
| | | GACCCGGAATAAGCACATCAACCCCAACGAGTGGTGGAAGGTGTACC |
| | | CTAGCAGCGTGACCGAGTTCAAGTTCCTGTTCGTGAGCGGCCACTTC |
| | | AAGGGCAACTACAAGGCCCAGCTGACCAGGCTGAACCGCAAAACCAA |
| | | CTGCAATGGCGCCGTGCTGAGCGTGGAGGAGCTGCTGATCGGCGGCG |
| | | AGATGATCAAAGCCGGCACCCTGACACTGGAGGAGGTGCGGCGCAAG |
| | | TTCAACAACGGCGAGATCAACTTC |
| 158 | Fok1 | CAGCTGGTCAAAAGTGAACTGGAGGAAAAAAAGAGCGAACTGAGACA |
| | (Right ZFN) | CAAACTGAAGTACGTGCCACACGAATATATTGAGCTGATTGAGATCG |
| | Codon | CGAGGAACTCAACACAGGACCGCATTCTGGAGATGAAAGTGATGGAG |
| | diversified (na) | TTTTTCATGAAAGTATATGGATATAGAGGAAAACACCTTGGGGGTAG |
| | Version 1 | CCGAAAGCCGGACGGGGCGATCTACACTGTGGGGTCACCAATTGATT |
| | | ATGGCGTAATTGTCGATACCAAAGCCTACAGTGGGGGGTACAATCTG |
| | | AGTATAGGACAGGCTGATGAAATGCAACGATACGTTAAGGAGAATCA |
| | | GACTAGGAATAAACATATCAATCCAAATGAATGGTGGAAAGTCTATC |
| | | CCAGCAGCGTGACAGAATTTAAATTTTTGTTTGTCAGTGGACACTTC |
| | | AAGGGAAATTATAAGGCCCAGCTGACTAGACTGAATAGGAAAACCAA |
| | | TTGTAATGGCGCAGTGCTTTCAGTGGAGGAACTGCTCATTGGAGGTG |
| | | AGATGATCAAGGCTGGAACCCTGACGCTGGAGGAGGTGCGGAGGAAG |
| | | TTTAACAATGGAGAAATTAACTTT |
| 159 | Fok1 | CAACTGGTGAAATCCGAACTGGAAGAAAAGAAATCAGAATTGCGGCA |
| | (Right ZFN) | TAAACTGAAGTATGTGCCCCATGAGTACATAGAACTGATCGAGATCG |
| | Codon | CAAGGAACTCTAGCCAGGACAGAATACTTGAAATGAAGGTCATGGAA |
| | diversified (na) | TTTTTTATGAAAGTGTACGGCTACAGAGGAAAACATTTGGGAGGCAG |
| | Version 2 | TCGAAAACCAGATGGCGCAATCTATACAGTCGGGTCCCCCATAGATT |
| | | ACGGAGTGATTGTCGACACAAAAGCCTATTCCGGAGGATATAACCTT |
| | | AGTATCGGCCAGGCCGACGAGATGCAACGCTATGTGAAAGAAAACCA |
| | | AACAAGAAATAAACATATCAATCCAAACGAGTGGTGGAAGGTATATC |
| | | CAAGCAGTGTCACAGAATTCAAATTCCTCTTCGTGAGTGGGCACTTT |
| | | AAAGGCAACTACAAAGCTCAATTGACCAGGCTCAATCGGAAAACTAA |
| | | TTGCAATGGCGCAGTCCTTAGCGTCGAAGAATTGCTGATTGGCGGGG |
| | | AAATGATTAAAGCAGGAACTTTGACCTTGGAGGAAGTAGGGAGAAAG |
| | | TTTAACAACGGCGAGATTAATTTT |
| 160 | Fok1 | CAGCTGGTGAAATCTGAGCTGGAAGAGAAGAAATCTGAACTGCGACA |
| | (Right ZFN) | TAAATTGAAGTACGTCCCACACGAGTACATCGAGTTGATCGAAATTG |
| | Codon | CCCGGAATAGCACCCAGGATAGAATATTGGAAATGAAAGTAATGGAG |
| | diversified (na) | TTTTTTATGAAGGTTTATGGTTACAGAGGCAAGCACCTTGGAGGAAG |
| | Version 3 | CAGGAAACCAGATGGGGCGATTTACACCGTTGGGAGTCCCATCGATT |
| | | ACGGAGTCATCGTGGACACAAAGGCCTATTCCGGAGGCTACAACCTC |
| | | AGTATCGGGCAAGCCGATGAGATGCAGAGATATGTTAAAGAAAATCA |
| | | GACGCGAAACAAGCACATTAACCCAAACGAATGGTGGAAAGTTTACC |
| | | CTAGCTCAGTGACAGAATTTAAGTTTCTGTTTGTCAGCGGCCACTTC |
| | | AAGGGGAATTATAAAGCACAACTGACCCGCCTGAACCGAAAAACCAA |
| | | CTGTAACGGTGCTGTGCTGAGTGTCGAAGAGTTGCTTATCGGAGGAG |
| | | AGATGATAAAGGCCGGCACACTGACGCTTGAAGAGGTACGGCGAAAA |
| | | TTCAATAACGGAGAGATTAATTTT |
| 161 | Fok1 | CAGCTGGTCAAATCAGAACTTGAAGAGAAAAAAAGCGAACTGAGACA |
| | (Right ZFN) | TAAACTGAAGTACGTGCCTCATGAATACATAGAGCTCATTGAAATAG |
| | Codon | CTAGGAATAGTACACAGGACAGGATACTTGAAATGAAGGTAATGGAA |
| | diversified (na) | TTTTTCATGAAGGTTTATGGATACCGGGGGAAACATCTCGGGGGCAG |
| | Version 4 | CAGAAAACCAGACGGAGCAATTTATACTGTCGGGAGTCCTATAGATT |
| | | ATGGCGTTATCGTCGATACAAAGGCCTATTCCGGTGGGTACAACCTC |
| | | TCAATTGGTCAGGCTGATGAGATGCAAAGATACGTCAAAGAAAACCA |
| | | AACCAGAAATAAACATATAAATCCCAATGAATGGTGGAAAGTATACC |
| | | CAAGTTCCGTGACTGAATTCAAGTTCCTTTTCGTGTCTGGCCACTTT |
| | | AAAGGAAATTATAAAGCACAATTGACTAGACTGAATAGAAAAACAAA |
| | | CTGTAACGGCGCAGTGCTGTCAGTGGAAGAACTGCTCATAGGTGGAG |
| | | AGATGATCAAGGCCGGGACACTTAGTCTTGAGGAAGTTAGAAGGAAG |
| | | TTCAACAACGGCGAAATCAACTTT |
| 162 | Fok1 | CAACTGGTCAAGTCAGAATTGGAAGAGAAAAAATCTGAGCTGAGGCA |
| | (Right ZFN) | CAAATTGAAATACGTTCCTCACGAGTATATTGAACTTATCGAGATAG |
| | Codon | CCCGCAATAGTAGACAAGATAGAATCTTGGAGATGAAAGTTATGGAA |
| | diversified (na) | TTCTTTATGAAAGTCTATGGCTATAGGGGAAAACACCTGGGGGGTAG |
| | Version 5 | CAGGAAACCTGATGGAGCTATCTATACCGTAGGATCACCTATTGATT |
| | | ATGGAGTAATTGTGGACACTAAGGCATATTCCGGAGGATATAATTTG |
| | | AGTATTGGTCAGGCCGACGAAATGCAACGATACGTGAAGGAAAATCA |
| | | GACCCGCAACAAACACATTAATCCCAATGAATGGTGGAAGGTATACC |
| | | CTAGTAGCGTTACAGAGTTTAAATTCCTTTTCGTCAGCGGCCACTTT |
| | | AAAGGAAATTATAAAGCACAACTCACCAGACTTAATCGAAAAACTAA |
| | | CTGTAACGGCGCCGTACTGTCAGTGGAGGAGCTGCTCATTGGAGGCG |
| | | AGATGATCAAGGCCGGTACTCTCACACTGGAAGAAGTTAGAAGAAAG |
| | | TTCAACAACGGGGAAATTAATTTC |
| 163 | Fok1 | CAGCTGGTCAAAAGCGAACTTGAAGAAAAGAAATCCGAGCTTAGACA |
| | (Right ZFN) | CAAACTGAAATACGTGCCTCACGAGTATATTGAGCTGATTGAAATAG |
| | Codon | CAAGGAATTCAACACAAGACAGGATCCTCGAAATGAAGGTTATGGAG |
| | diversified (na) | TTTTTCATGAAAGTTTACGGCTACAGAGGGAAGCATCTGGGCGGATC |
| | Version 6 | AAGAAAACCAGACGGCGCAATCTACACAGTTGGATCCCCAATAGATT |
| | | ACGGAGTGATTGTTGACACCAAGGCTTATTCAGGAGGTTACAATCTG |
| | | TCCATTGGTCAGGCCGATGAAATGCAAAGATATGTTAAGGAAAATCA |
| | | AACTCGAAACAAACACATTAATCCAAACGAATGGTGGAAAGTATATC |
| | | CAAGCTCCGTCACTGAATTTAAATTTTTGTTTGTATCCGGACATTTT |
| | | AAGGGCAACTATAAGGCTCAACTGACCAGACTGAATAGGAAGACCAA |
| | | TTGTAACGGAGCTGTACTCAGCGTGGAAGAACTGCTTATTGGAGGCG |
| | | AAATGATTAAGGCTGGCACACTTACACTCGAAGAAGTTAGAAGAAAA |
| | | TTCAACAATGGTGAGATAAACTTC |
| 164 | Fok1 | CAGCTGGTGAAGAGCGAGCTGGAGGAGAAGAAGTCCGAGCTGCGGCA |
| | (Left ZFN) | CAAGCTGAAGTACGTGCCCCACGAGTACATCGAGCTGATCGAGATCG |
| | Not diversified | CCAGGAACAGCACCCAGGACCGCATCCTGGAGATGAAGGTGATGGAG |
| | (na) | TTCTTCATGAAGGTGTACGGCTACAGGGGAAAGCACCTGGGCGGAAG |
| | | CAGAAAGCCTGACGGCGCCATCTATACAGTGGGCAGCCCCATCGATT |
| | | ACGGCGTGATCGTGGACACAAAGGCCTACAGCGGCGGCTACAATCTG |
| | | CCTATCGGCCAGGCCGACGAGATGGAGAGATACGTGGAGGAGAACCA |
| | | GACCCGGGATAAGCACCTCAACCCCAACGAGTGGTGGAAGGTGTACC |
| | | CTAGCAGCGTGACCGAGTTCAAGTTCCTGTTCGTGAGCGGCCACTTC |
| | | AAGGGCAACTACAAGGCCCAGCTGACCAGGCTGAACCACATCACCAA |
| | | CTGCGACGGCGCCGTGCTGAGCGTGGAGGAGCTGCTGATCGGCGGCG |
| | | AGATGATCAAAGCCGGCACCCTGACACTGGAGGAGGTGCGGCGCAAG |
| | | TTCAACAACGGCGAGATCAACTTCAGATCT |
| 165 | Fok1 | CAGCTTGTGAAGTCCGAACTGGAGGAAAAGAAGAGCGAACTGCGCCA |
| | (Left ZFN) | CAAATTGAAATACGTTCCGCATGAGTACATAGAGCTCATTGAAATCG |
| | Codon | CTAGAAACTCTACCCAAGACAGGATACTGGAAATGAAAGTGATGGAA |
| | diversified (na) | TTTTTCATGAAAGTTTATGGTTATAGGGGCAAACATCTGGGTGGCTC |
| | Version 1 | TCGCAAGCCCGATGGGGCCATTTATACTGTCGGCTCACCTATCGACT |
| | | ATGGCGTCATTGTGGATACCAAGGCTTATTCTGGAGGATACAACCTG |
| | | CCCATCGGACAAGCAGACGAAATGGAAAGATACGTCGAGGAGAATCA |
| | | AACCCGAGACAAGCATCTGAACCCAAACGAGTGGTGGAAAGTGTACC |
| | | CGAGCAGCGTTACTGAGTTCAAATTTCTCTTTGTAAGCGGACATTTT |
| | | AAAGGGAATTACAAAGCACAACTGACTAGGCTGAACCATATAACCAA |
| | | CTGTGACGGGGCCGTATTGAGTGTGGAAGAGCTTCTGATTGGAGGAG |
| | | AGATGATTAAGGCTGGCACACTGACTCTCGAAGAAGTGAGGCGCAAA |
| | | TTCAATAACGGTGAAATCAACTTCCGGTCT |
| 166 | Fok1 | CAGCTGGTGAAGAGTGAATTGGAAGAAAAAAAGTCAGAGCTGAGACA |
| | (Right ZFN) | CAAACTGAAATATGTTCCACACGAGTACATCGAGCTTATCGAGATAG |
| | Codon | CAAGAAACTCCACCCAGGACAGAATTTTGGAAATGAAAGTTATGGAA |
| | diversified (na) | TTCTTTATGAAAGTGTATGGCTACAGGGGTAAACATCTGGGGGGATC |
| | Version 2 | AAGAAAGCCTGATGGTGCAATTTACACAGTGGGCTCTCCTATCGACT |
| | | ACGGTGTGATCGTGGATACAAAGGCCTACTCTGGAGGATATAATTTG |
| | | CCTATTGGACAAGCCGATGAAATGGAAAGATATGTGGAGGAAAACCA |
| | | GAGTCGCGATAAGCACCTGAACCCAAATGAATGGTGGAAAGTGTACC |
| | | CTTCATCTGTTACCGAATTTAAATTTTTGTTCGTTTCCGGGCATTTC |
| | | AAGGGGAACTACAAGGCACAGCTGACGAGACTGAATCACATCACGAA |
| | | CTGCGACGGCGCTGTACTGTCCGTGGAAGAGCTTTTGATCGGGGGCG |
| | | AAATGATTAAGGCCGGCACACTGACGCTGGAGGAGGTGCGGCGAAAA |
| | | TTTAATAATGGCGAGATCAATTTTAGGAGT |
| 167 | Fok1 | CAACTGGTCAAGTCCGAACTGGAGGAAAAAAAAAGTGAGCTGCGACA |
| | (Right ZFN) | CAAGTTGAAGTACGTACCACACGAATACATCGAGCTGATTGAGATAG |
| | (na) | CACGGAACTCTAGCCAGGATAGAATACTGGAGATGAAAGTTATGGAA |
| | Codon | TTCTTTATGAAGGTGTACGGATACAGGGGGAAGCATCTTGGCGGGAG |
| | diversified | CCGGAAACCAGACGGAGCAATCTATACCGTCGGGTCACCTATAGACT |
| | Version 3 | ATGGAGTTATTGTCGATACAAAGGCCTATTCAGGAGGTTATAATCTG |
| | | CCAATCGGCCAAGCCGACGAGATGGAGAGGTACGTGGAGGAAAATCA |
| | | GACCAGAGACAAGCACCTGAACCCTAATGAATGGTGGAAAGTGTACC |
| | | CTAGCAGCGTCACTGAGTTCAAATTCCTGTTCGTCAGCGGTCATTTT |
| | | AAAGGAAATTATAAAGCCCAGCTCACTAGACTCAACCATATTACAAA |
| | | CTGCGACGGAGCCGTACTTAGCGTTGAAGAGTTGCTTATCGGAGGAG |
| | | AGATGATCAAAGCCGGAACCCTCACACTTGAAGAAGTGCGAAGAAAA |
| | | TTCAATAACGGAGAGATAAATTTTAGGAGT |
| 168 | Fok1 | CAGCTGGTTAAATCCGAACTTGAAGAAAAAAAAAGTGAACTGCGGCA |
| | (Right ZFN) | TAAACTGAAGTATGTCCCCCATGAATATATCGAACTGATAGAAATCG |
| | Codon | CCCGAAATAGCACCCAAGATAGAATCCTCGAAATGAAGGTTATGGAA |
| | diversified (na) | TTTTTCATGAAGGTCTATGGATATAGGGGCAAGCACCTTGGCGGATC |
| | Version 4 | CCGGAAACCTGATGGAGCTATCTACACAGTGGGCTCACCAATAGACT |
| | | ATGGAGTTATCGTCGATACAAAAGCATACAGCGGAGGATACAATTTG |
| | | CCAATAGGTCAAGCAGATGAGATGGAAAGATACGTGGAGGAAAACCA |
| | | AACAAGAGATAAGCATCTGAACCCCAACGAATGGTGGAAAGTGTACC |
| | | CCAGTTCTGTAACCGAATTTAAGTTCTTGTTCGTTTCAGGTCACTTC |
| | | AAGGGTAATTACAAGGCTCAACTGACTAGACTCAACCATATTACAAA |
| | | TTGCGATGGTGCTGTGCTTTCCGTGGAAGAATTGCTGATTGGTGGAG |
| | | AGATGATAAAAGCTGGTACCCTCACCTTGGAAGAAGTGCGCAGAAAA |
| | | TTCAATAATGGCGAGATCAACTTCCGAAGT |
| 169 | Fok1 | CAACTGGTGAAAAGTGAACTGGAGGAAAAAAAATCTGAGCTGAGACA |
| | (Right ZFN) | TAAACTGAAATACGTACCACATGAATACATAGAACTTATAGAAATAG |
| | Codon | CTAGGAACTCCACCCAGGACAGAATACTTGAAATGAAGGTCATGGAG |
| | diversified (na) | TTTTTTATGAAAGTTTACGGATACAGGGGCAAACACCTTGGAGGGTC |
| | Version 5 | TCGGAAGCCTGATGGCGCAATTTATACCGTGGGTAGCCCTATAGATT |
| | | ATGGAGTGATTGTGGATACAAAGGCTTACAGTGGCGGCTATAATTTG |
| | | CCTATCGGACAGGCCGATGAGATGGAAAGATACGTTGAAGAAAACCA |
| | | AACACGAGATAAGCATCTGAACCCCAATGAATGGTGGAAAGTGTATC |
| | | CTTCAAGCGTTACCGAGTTTAAGTTCCTCTTCGTTTCTGGGCATTTC |
| | | AAGGGGAACTACAAAGCTCAGCTTACAAGACTCAACCACATAACCAA |
| | | TTGTGATGGAGCAGTCCTCAGCGTGGAAGAACTCCTTATTGGGGGTG |
| | | AGATGATTAAAGCAGGGACCCTTACTCTTGAAGAGGTTAGAAGAAAA |
| | | TTCAATAACGGAGAGATTAATTTTAGAAGT |
| 170 | Fok1 | CAGCTGGTCAAGTCTGAACTGGAAGAAAAAAAAAGCGAACTGCGGCA |
| | (Right ZFN) | TAAACTCAAATACGTCCCACATGAATACATTGAGCTCATCGAAATTG |
| | Codon | CTAGAAACTCTAGTCAAGATAGGATATTGGAGATGAAGGTAATGGAA |
| | diversified (na) | TTCTTCATGAAGGTTTATGGATATAGAGGAAAACATCTTGGAGGCAG |
| | Version 6 | TAGGAAACCCGATGGCGCTATCTACACCGTAGGGAGTCCAATCGACT |
| | | ACGGCGTGATTGTTGACACCAAAGCCTATTCTGGAGGGTATAATCTC |
| | | CCAATTGGACAGGCAGATGAGATGGAAAGATATGTAGAAGAAAATCA |
| | | GACAAGAGATAAGCACCTTAACCCTAACGAGTGGTGGAAAGTGTACC |
| | | CAAGCAGTGTTACTGAATTTAAATTTCTTTTTGTATCAGGACACTTT |
| | | AAAGGCAATTACAAAGCACAACTGACCAGACTCAATCACATTACCAA |
| | | TTGCGACGGAGCCGTACTGAGCGTGGAGGAGTTGCTGATCGGAGGCG |
| | | AAATGATTAAAGCTGGCACTCTGACCCTGGAAGAAGTAAGAAGAAAG |
| | | TTCAATAATGGAGAAATAAACTTTCGCTCC |
| 171 | Fok1 | QLVKSELEEKKSELRHKLKYVPHEYIELIEIARNSTQDRILEMKVME |
| | (Right ZFN) | FFMKVYGYRGKHLGGSRKPDGAIYTVGSPIDYGVIVDTKAYSGGYNL |
| | (aa) | SIGQADEMQRYVKENQTRNKHINPNEWWKVYPSSVTEFKFLFVSGHF |
| | | KGNYKAQLTRLNRKTNCNGAVLSVEELLIGGEMIKAGTLTLEEVRRK |
| | | FNNGEINF |
| 172 | Fok1 | QLVKSELEEKKSELRHKLKYVPHEYIELIEIARNSTQDRILEMKVME |
| | (Left ZFN)(aa) | FFMKVYGYRGKHLGGSRKPDGAIYTVGSPIDYGVIVDTKAYSGGYNL |
| | | PIGQADEMERYVEENQTRDKHLNPNEWWKVYPSSVTEFKFLFVSGHF |
| | | KGNYKAQLTRLNHITNCDGAVLSVEELLIGGEMIKAGTLTLEEVRRK |
| | | FNNGEINFRS |

TABLE 5
Exemplary 2-in-1 Constructs

| SEQ | | |
|---|---|---|
| ID | Feature/ | |
| NO | Description | Annotated Nucleic Acid (na) Sequence |
| 35 | GUS130- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (R1-L)(na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTACTCATGTCCCTAAAATGGGCAAACATTGC |
| | Version 1 | AAGCAGCAAACAGCAAACACACAGCCCTCCCTGCCTGCTGACCT |
| | ZFN-L | TGGAGCTGGGGCAGAGGTCAGAGACCTCTCTGGGCCCATGCCAC |
| | Not | CTCCAACATCCACTCGACCCCTTGGAATTTCGGTGGAGAGGAGC |
| | diversified | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 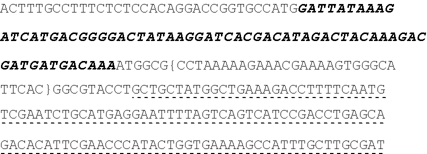 |
| | | 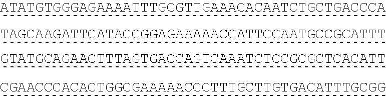 |
| | | 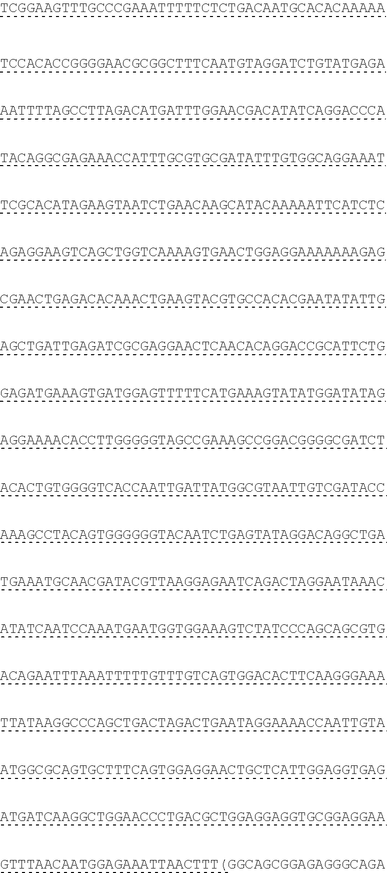 |
| | | GGAAGCCTGCTCACCTGCGGTGACGTGGAGGAAAACCCTGGCCC |
| | | 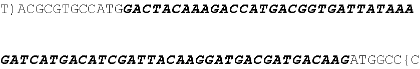 |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | 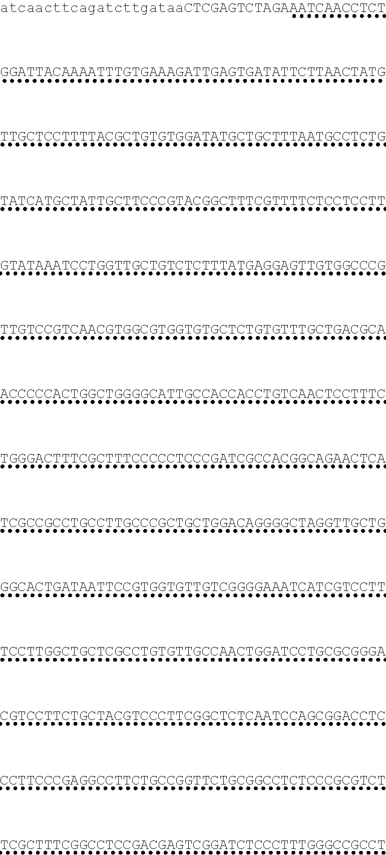 |
| | | 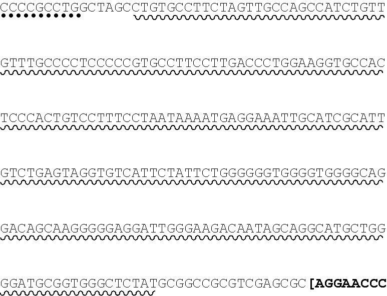 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 36 | GUS131- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (R2-L)(na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 2 | AAGCAGCAAACAGCAAACACACAGCCCTCCCTGCCTGCTGACCT |
| | ZFN-L | TGGAGCTGGGGCAGAGGTCAGAGACCTCTCTGGGCCCATGCCAC |
| | Not | CTCCAACATCCACTCGACCCCTTGGAATTTCGGTGGAGAGGAGC |
| | diversified | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 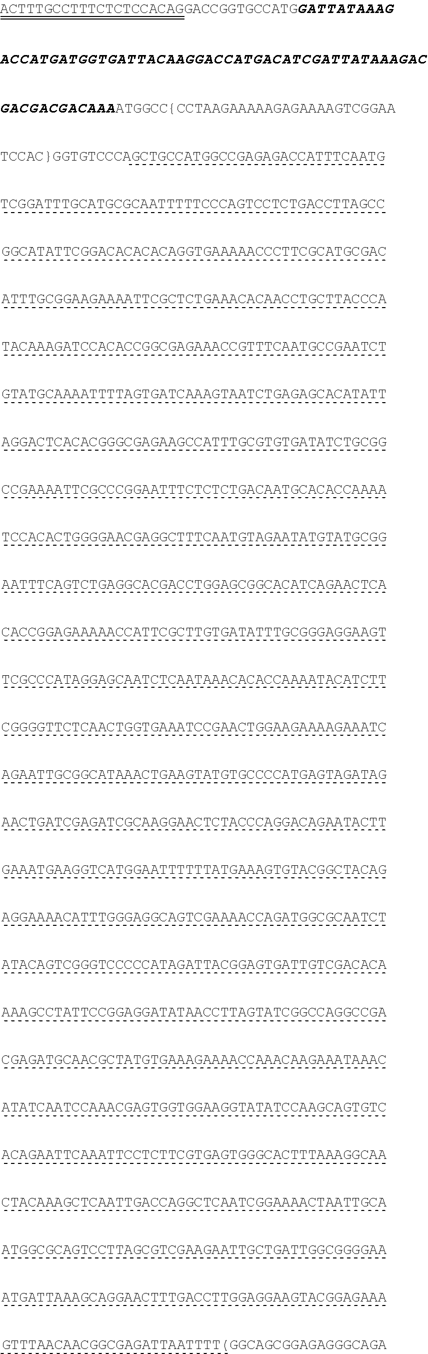 |
| | | GGAAGCCTGCTCACCTGCGGTGACGTGGAGGAAAACCCTGGCCC |
| | | 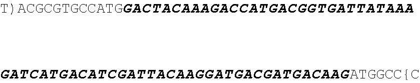 |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | 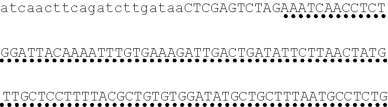 |
| | | 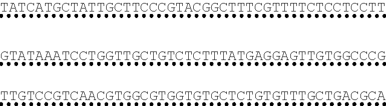 |
| | | 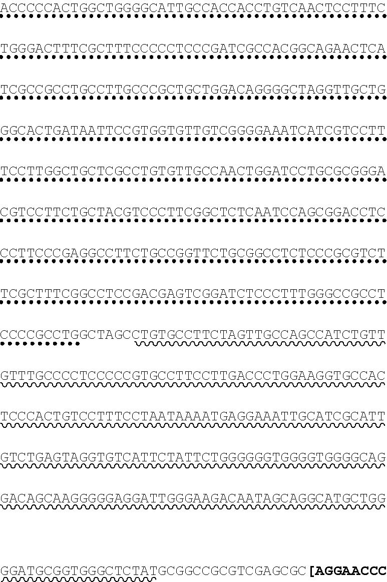 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 37 | GUS132- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (R3-L)(na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 3 | AAGCAGCAAACAGCAAACACACAGCCCTCCCTGCCTGCTGACCT |
| | ZFN-L | TGGAGCTGGGGCAGAGGTCAGAGACCTCTCTGGGCCCATGCCAC |
| | Not | CTCCAACATCCACTCGACCCCTTGGAATTTCGGTGGAGAGGAGC |
| | diversified | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 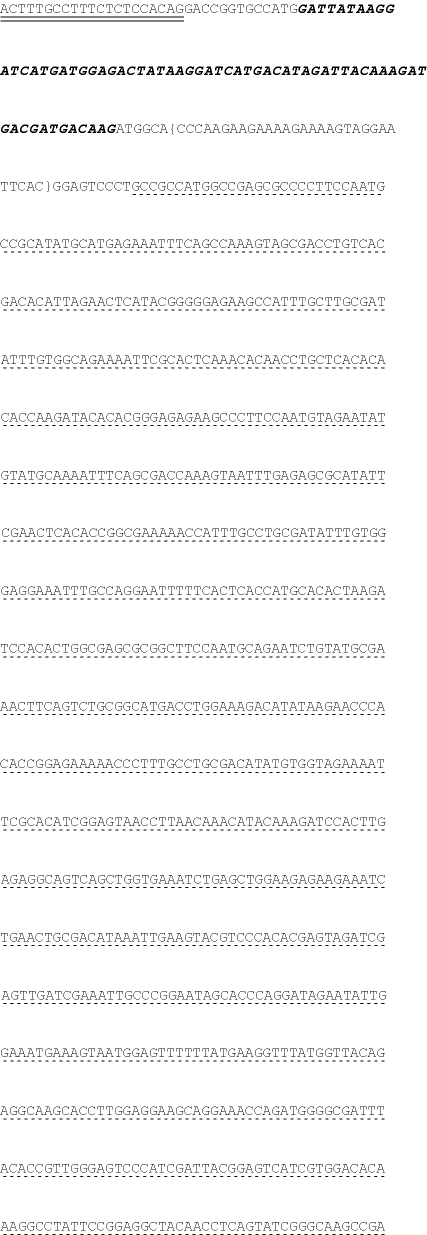 |
| | | 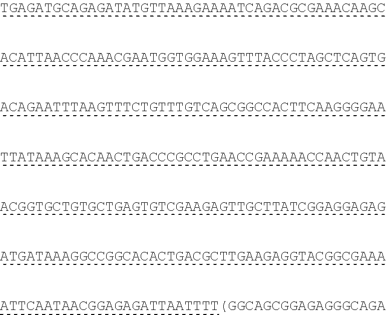 |
| | | GGAAGCCTGCTCACCTGCGGTGACGTGGAGGAAAACCCTGGCCC |
| | | 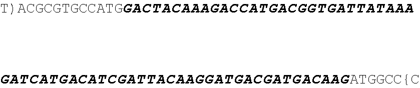 |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | 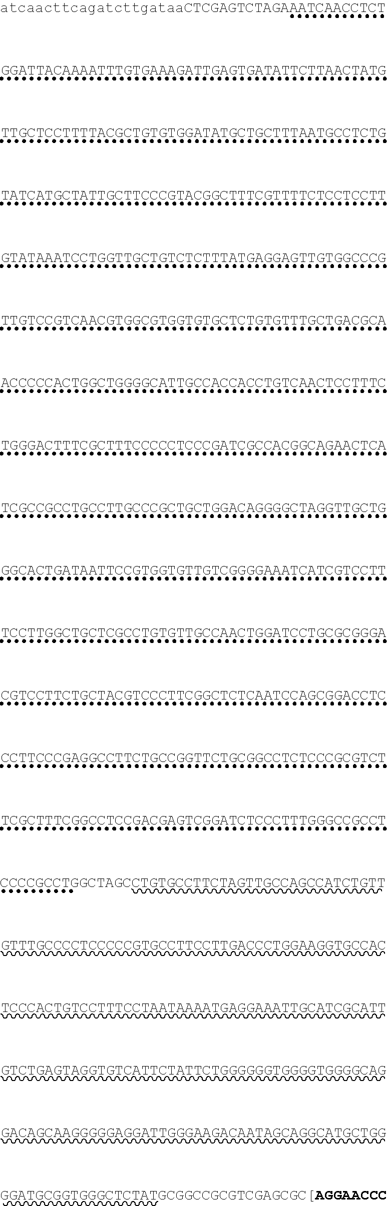 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 38 | GUS133- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (R1_HL-L) | GCGGCCTAAGCTTGAGCTCTTCGAAAGGCTCAGAGGCACACAGG |
| | (na) | AGTTTCTGGGCTCACCCTGCCCCCTTCCAACCCCTCAGTTCCCA |
| | ZFN-R | TCCTCCAGCAGCTGTTTGTGTGCTGCCTCTGAAGTCCACACTGA |
| | Codon | ACAAACTTCAGCCTAGTCATGTCCCTAAAATGGGCAAACATTGC |
| | diversified | AAGCAGCAAACAGCAAACACACAGCCCTCCCTGCCTGCTGACCT |
| | Version 4 | TGGAGCTGGGGCAGAGGTCAGAGACCTCTCTGGGCCCATGCCAC |
| | ZFN-L | CTCCAACATCCACTCGACCCCTTGGAATTTCGGTGGAGAGGAGC |
| | Not | AGAGGTTGTCCTGGCGTGGTTTAGGTAGTGTGAGAGGGGTCCCG |
| | diversified | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 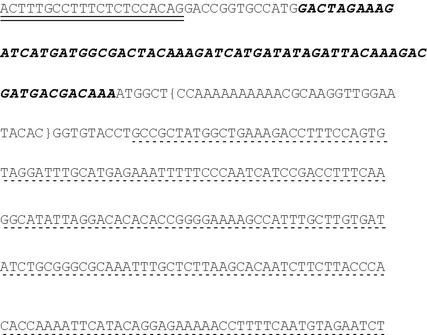 |
| | | 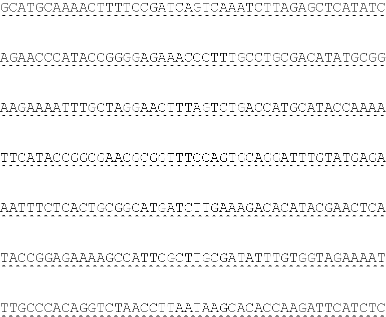 |
| | | 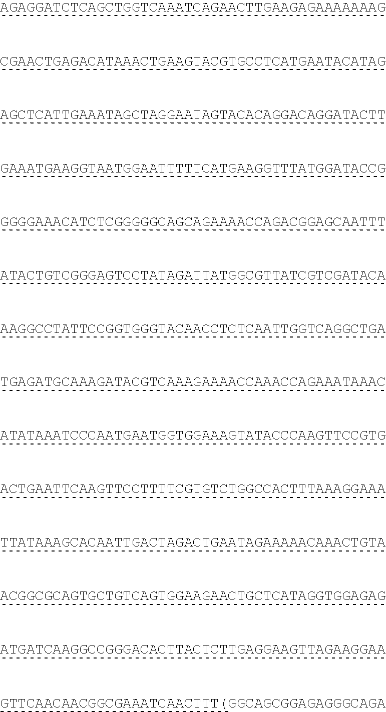 |
| | | GGAAGCCTGCTCACCTGCGGTGACGTGGAGGAAAACCCTGGCCC |
| | | 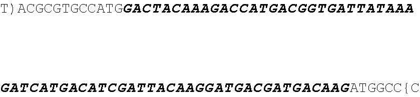 |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | 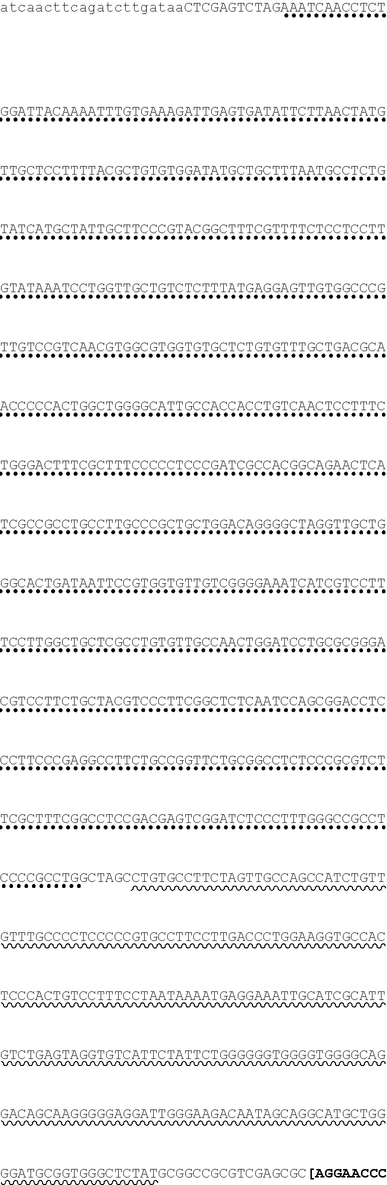 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 39 | GUS134- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (R2_HL-L) | GCGGCCTAAGCTTGAGCTCTTCGAAAGGCTCAGAGGCACACAGG |
| | (na) | AGTTTCTGGGCTCACCCTGCCCCCTTCCAACCCCTCAGTTCCCA |
| | ZFN-R | TCCTCCAGCAGCTGTTTGTGTGCTGCCTCTGAAGTCCACACTGA |
| | Codon | ACAAACTTCAGCCTAGTCATGTCCCTAAAATGGGCAAACATTGC |
| | diversified | AAGCAGCAAACAGCAAACACACAGCCCTCCCTGCCTGCTGACCT |
| | Version 5 | TGGAGCTGGGGCAGAGGTCAGAGACCTCTCTGGGCCCATGCCAC |
| | ZFN-L | CTCCAACATCCACTCGACCCCTTGGAATTTCGGTGGAGAGGAGC |
| | Not | AGAGGTTGTCCTGGCGTGGTTTAGGTAGTGTGAGAGGGGTCCCG |
| | diversified | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 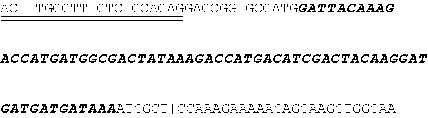 |
| | | 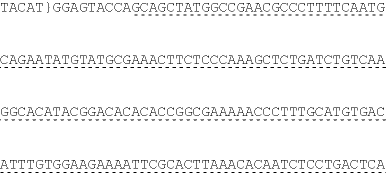 |
| | | 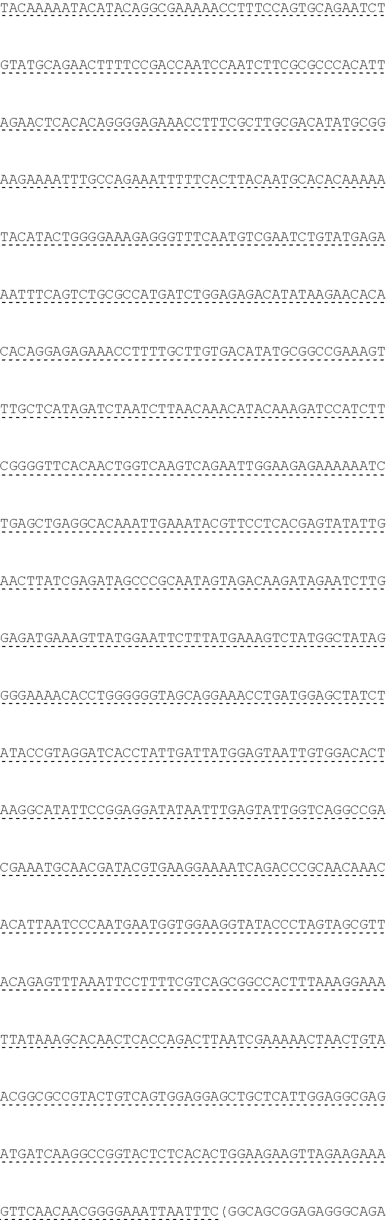 |
| | | GGAAGCCTGCTCACCTGCGGTGACGTGGAGGAAAACCCTGGCCC |
| | | 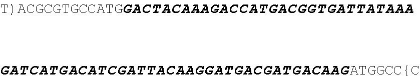 |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | 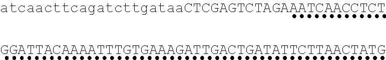 |
| | | 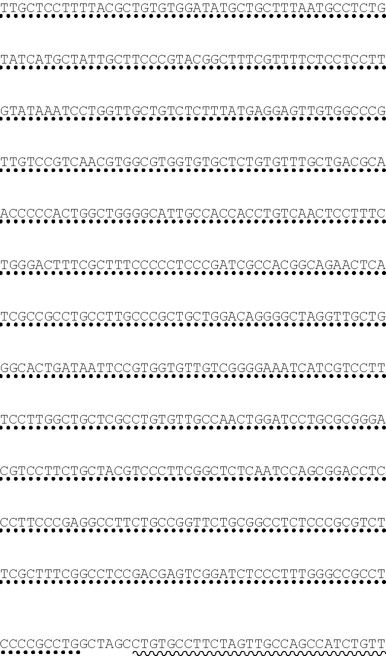 |
| | | 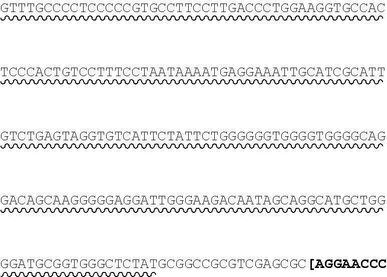 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 40 | GUS135- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (R3_HL-L) | GCGGCCTAAGCTTGAGCTCTTCGAAAGGCTCAGAGGCACACAGG |
| | (na) | AGTTTCTGGGCTCACCCTGCCCCCTTCCAACCCCTCAGTTCCCA |
| | ZFN-R | TCCTCCAGCAGCTGTTTGTGTGCTGCCTCTGAAGTCCACACTGA |
| | Codon | ACAAACTTCAGCCTAGTCATGTCCCTAAAATGGGCAAACATTGC |
| | diversified | AAGCAGCAAACAGCAAACACACAGCCCTCCCTGCCTGCTGACCT |
| | Version 6 | TGGAGCTGGGGCAGAGGTCAGAGACCTCTCTGGGCCCATGCCAC |
| | ZFN-L | CTCCAACATCCACTCGACCCCTTGGAATTTCGGTGGAGAGGAGC |
| | Not | AGAGGTTGTCCTGGCGTGGTTTAGGTAGTGTGAGAGGGGTCCCG |
| | diversified | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 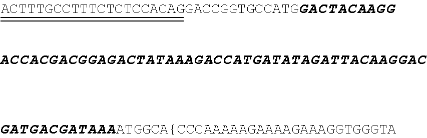 |
| | | 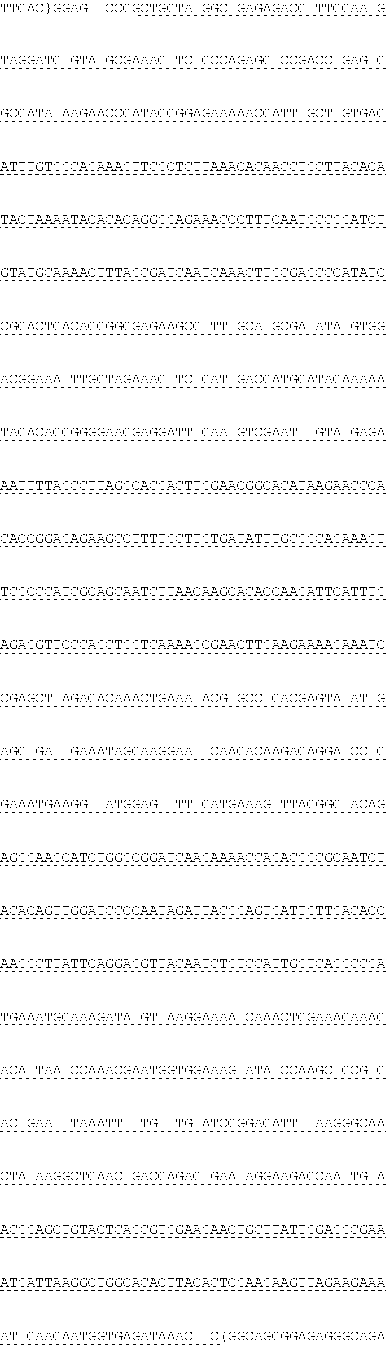 |
| | | GGAAGCCTGCTCACCTGCGGTGACGTGGAGGAAAACCCTGGCCC |
| | | 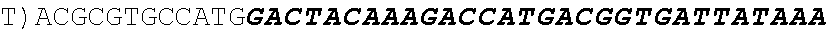 |
| | | 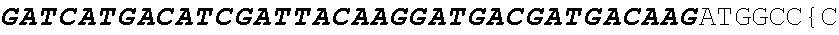 |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | 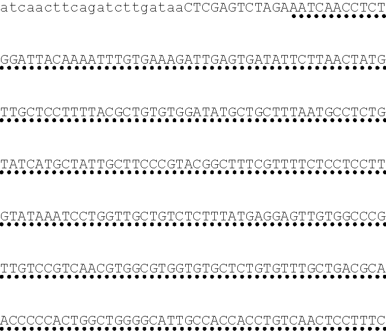 |
| | | 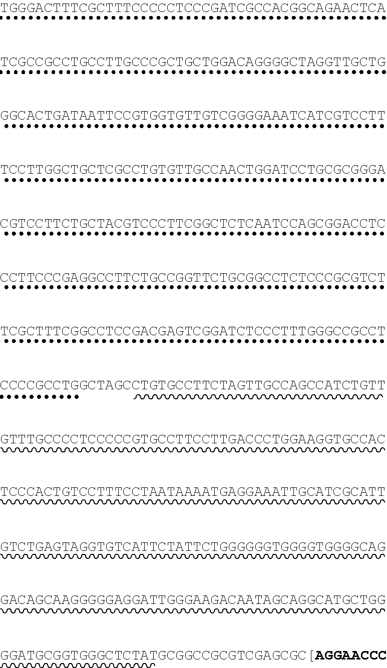 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 41 | GUS136- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (R-L)(na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Not | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | ZFN-L | AAGCAGCAAACAGCAAACACACAGCCCTCCCTGCCTGCTGACCT |
| | Not | TGGAGCTGGGGCAGAGGTCAGAGACCTCTCTGGGCCCATGCCAC |
| | diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 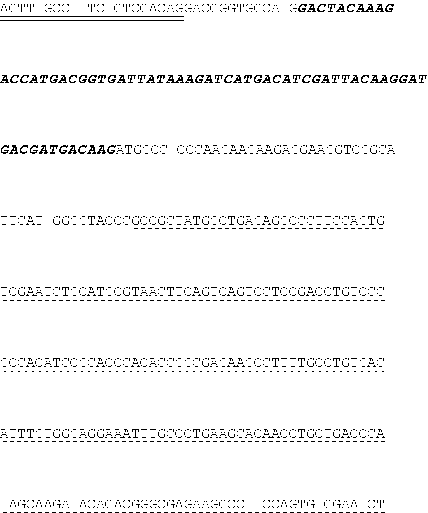 |
| | | 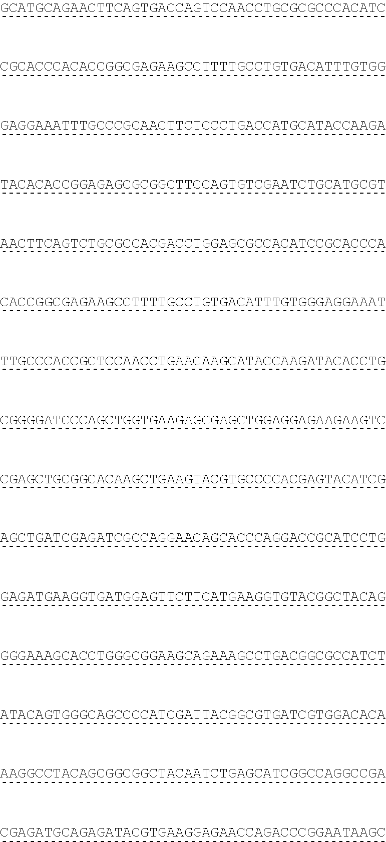 |
| | | 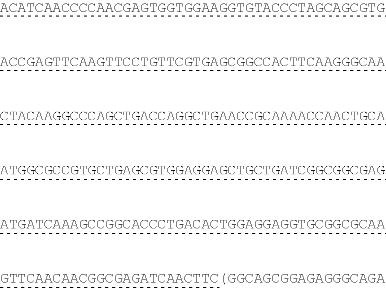 |
| | | GGAAGCCTGCTCACCTGCGGTGACGTGGAGGAAAACCCTGGCCC |
| | | 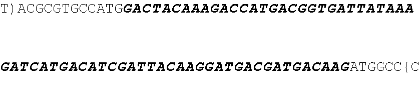 |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | 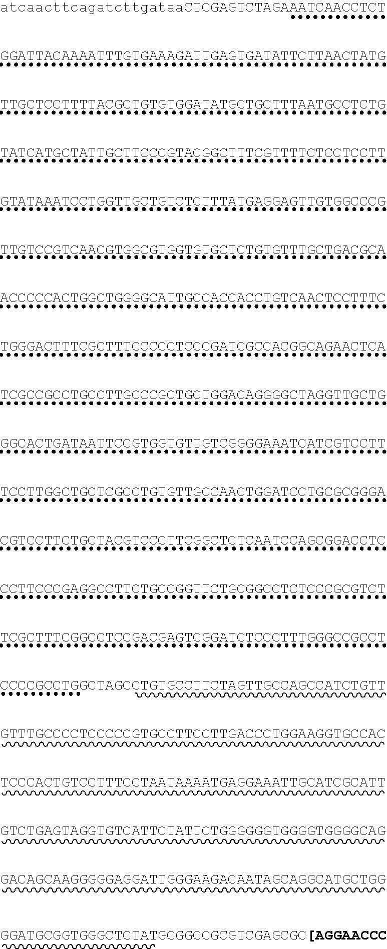 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 42 | GUS140- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (L1-R)(na) | GCGGCCTAAGCTTGAGCTCTTCGAAAGGCTCAGAGGCACACAGG |
| | ZFN-L | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTACTCATGTCCCTAAAATGGGCAAACATTGC |
| | Version 1 | AAGCAGCAAACAGCAAACACACAGCCCTCCCTGCCTGCTGACCT |
| | ZFN-R | TGGAGCTGGGGCAGAGGTCAGAGACCTCTCTGGGCCCATGCCAC |
| | Not | CTCCAACATCCACTCGACCCCTTGGAATTTCGGTGGAGAGGAGC |
| | diversified | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 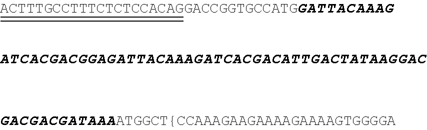 |
| | | TCCAT}GGTGTACCCgcagcaatggccgaacgacccttccaatg |
| | | cagaatatgtatgcagaatttttctcagagcgggaacctggcga |
| | | ggcacataagaacccatacaggagagaagccattcgcatgcgat |
| | | atttgcggtagaaaatttgcactcaaacaaaatctctgtatgca |
| | | cactaaaatccatacaggtgaaaagccttttcagtgcaggattt |
| | | gtatgcaaaaatttgcttggcaaagtaacttgcagaaccacaca |
| | | aagatacacacaggagagaaacccttccaatgccgaatctgtat |
| | | gcgcaacttcagtacatccggaaatttgactagacatattagga |
| | | cccacaccggcgagaagccatttgcctgcgatatttgtggacgg |
| | | aaattcgcacgacgcagccatctgaccagtcatactaagattca |
| | | tctccgcggcagccagcttgtgaagtccgaactggaggaaaaga |
| | | agagcgaactgcgccacaaattgaaatacgttccgcatgagtac |
| | | atagagctcattgaaatcgctagaaactctacccaagacaggat |
| | | actggaaatgaaagtgatggaatttttcatgaaagtttatggtt |
| | | ataggggcaaacatctgggtggctctcgcaagcccgatggggcc |
| | | atttatactgtcggctcacctatcgactatggcgtcattgtgga |
| | | taccaaggcttattctggaggatacaacctgcccatcggacaag |
| | | cagacgaaatggaaagatacgtcgaggagaatcaaacccgagac |
| | | aagcatctgaacccaaacgagtggtggaaagtgtacccgagcag |
| | | cgttactgagttcaaatttctctttgtaagcggacattttaaag |
| | | ggaattacaaagcacaactgactaggctgaaccatataaccaac |
| | | tgtgacggggccgtattgagtgtggaagagcttctgattggagg |
| | | agagatgattaaggctggcacactgactctcgaagaagtgaggc |
| | | gcaaattcaataacggtgaaatcaacttccggtct(GGCAGCGG |
| | | AGAGGGCAGAGGAAGCCTGCTCACCTGCGGTGACGTGGAGGAAA |
| | | 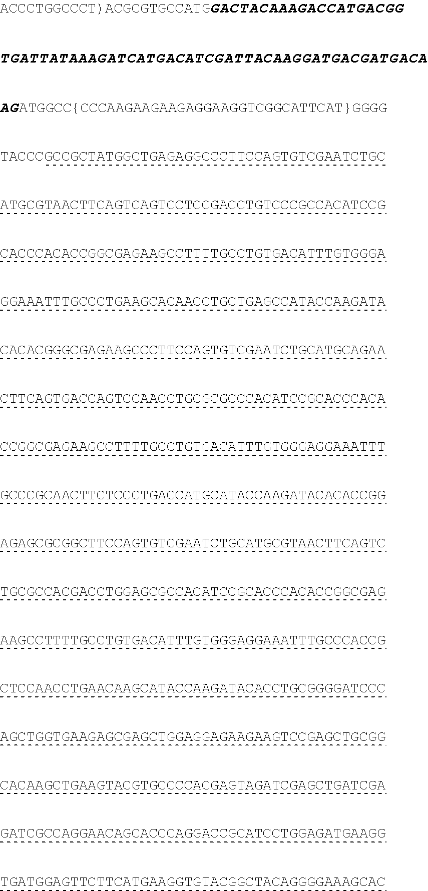 |
| | | 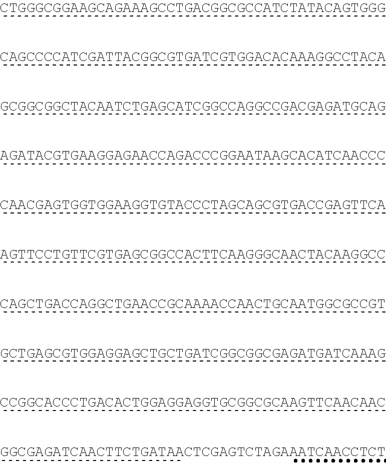 |
| | | 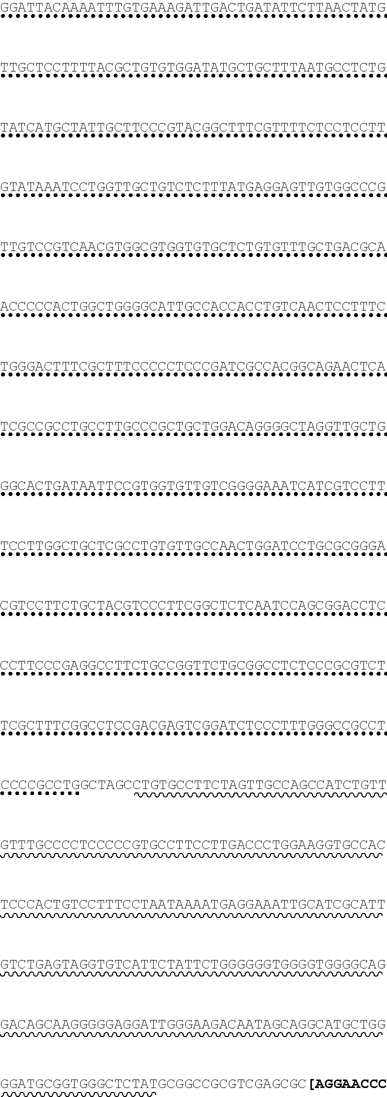 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 49 | GUS141- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (L2-R)(na) | GCGGCCTAAGCTTGAGCTCTTCGAAAGGCTCAGAGGCACACAGG |
| | ZFN-L | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 2 | AAGCAGCAAACAGCAAACACACAGCCCTCCCTGCCTGCTGACCT |
| | ZFN-R | TGGAGCTGGGGCAGAGGTCAGAGACCTCTCTGGGCCCATGCCAC |
| | Not | CTCCAACATCCACTCGACCCCTTGGAATTTCGGTGGAGAGGAGC |
| | diversified | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 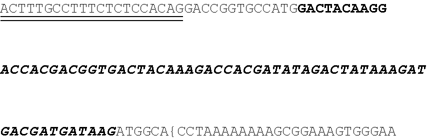 |
| | | TTCAC}GGCGTGCCCgccgccatggcagagagaccctttcaatg |
| | | tagaatctgtatgcaaaatttctctcagagtggtaaccttgcaa |
| | | gacacatcagaactcatacaggtgagaagccgtttgcatgtgac |
| | | atttgcggtaggaaatttgccttgaaacagaatctttgtatgca |
| | | cacaaaaatccatactggtgaaaagccattccaatgccgcatct |
| | | gtatgcaaaaattcgcgtggcagtccaatttgcagaaccatacc |
| | | aagattcacacgggagaaaaaccatttcagtgccgcatctgcat |
| | | gcgcaacttttctacatcaggaaaccttacacgacatattcgga |
| | | cgcacactggagaaaaaccatttgcttgtgacatatgcggccga |
| | | aaatttgccagacgctctcatctcacctcacatactaagattca |
| | | tttgcgcggaagtcagctggtgaagagtgaattggaagaaaaaa |
| | | agtcagagctgagacacaaactgaaatatgttccacacgagtac |
| | | atcgagcttatcgagatagcaagaaactccacccaggacagaat |
| | | tttggaaatgaaagttatggaattctttatgaaagtgtatggct |
| | | acaggggtaaacatctggggggatcaagaaagcctgatggtgca |
| | | atttacacagtgggctctcctatcgactacggtgtgatcgtgga |
| | | tacaaaggcctactctggaggatataatttgcctattggacaag |
| | | ccgatgaaatggaaagatatgtggaggaaaaccagactcgcgat |
| | | aagcacctgaacccaaatgaatggtggaaagtgtacccttcatc |
| | | tgttaccgaatttaaatttttgttcgtttccgggcatttcaagg |
| | | ggaactacaaggcacagctgacgagactgaatcacatcacgaac |
| | | tgcgacggcgctgtactgtccgtggaagagcttttgatcggggg |
| | | cgaaatgattaaggccggcacactgacgctggaggaggtgcggc |
| | | gaaaatttaataatggcgagatcaattttaggagt(GGCAGCGG |
| | | AGAGGGCAGAGGAAGCCTGCTCACCTGCGGTGACGTGGAGGAAA |
| | | 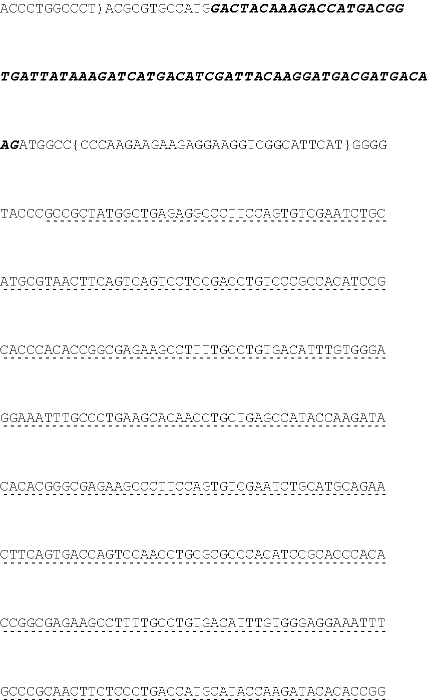 |
| | | 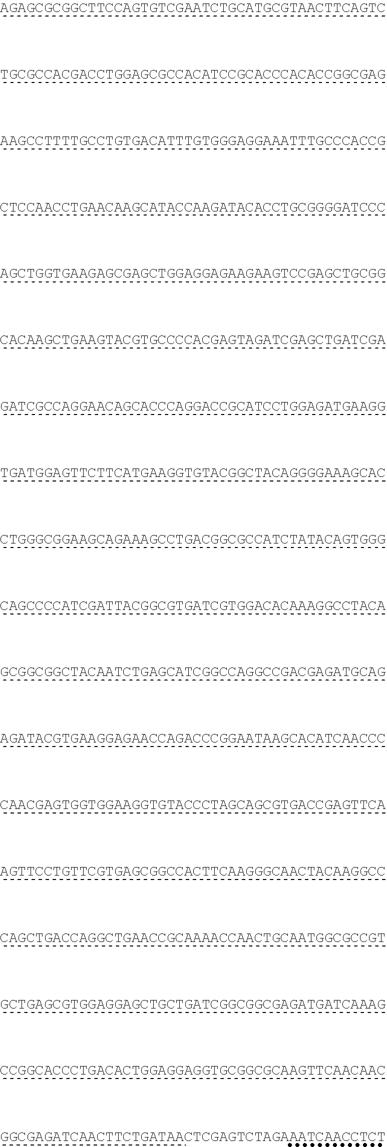 |
| | | 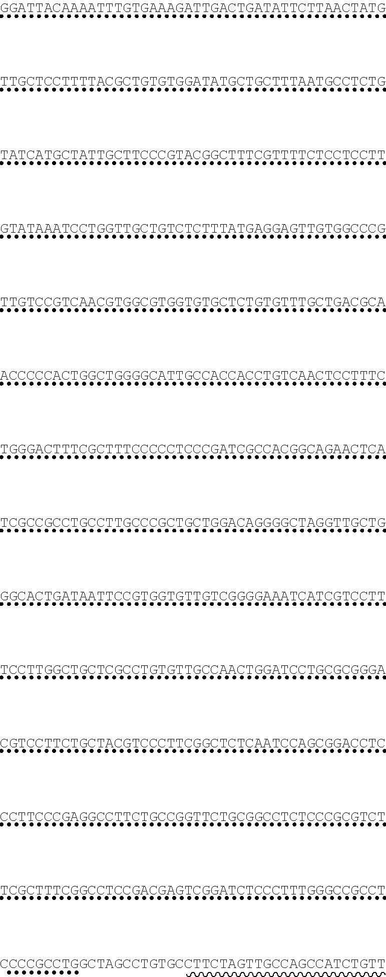 |
| | | 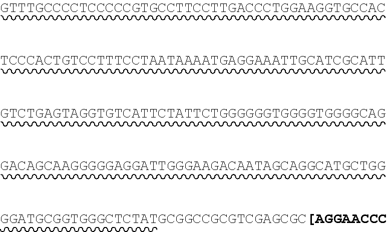 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 43 | GUS143- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (L1_HL-R) | GCGGCCTAAGCTTGAGCTCTTCGAAAGGCTCAGAGGCACACAGG |
| | (na) | AGTTTCTGGGCTCACCCTGCCCCCTTCCAACCCCTCAGTTCCCA |
| | ZFN-L | TCCTCCAGCAGCTGTTTGTGTGCTGCCTCTGAAGTCCACACTGA |
| | Codon | ACAAACTTCAGCCTAGTCATGTCCCTAAAATGGGCAAACATTGC |
| | diversified | AAGCAGCAAACAGCAAACACACAGCCCTCCCTGCCTGCTGACCT |
| | Version 4 | TGGAGCTGGGGCAGAGGTCAGAGACCTCTCTGGGCCCATGCCAC |
| | ZFN-R | CTCCAACATCCACTCGACCCCTTGGAATTTCGGTGGAGAGGAGC |
| | Not | AGAGGTTGTCCTGGCGTGGTTTAGGTAGTGTGAGAGGGGTCCCG |
| | diversified | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 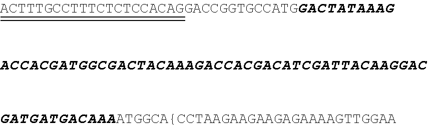 |
| | | TACAT}GGAGTCCCCgcagcaatggccgagagaccttttcagtg |
| | | caggatttgtatgcaaaacttctctcagtccggtaacctggccc |
| | | ggcacatacgaacacataccggcgaaaaaccctttgcttgcgac |
| | | atctgcggaagaaagttcgctcttaaacagaacctgtgcatgca |
| | | tacaaaaattcatacaggtgagaagccattccaatgcagaatat |
| | | gtatgcagaaattcgcctggcaaagcaacctgcaaaaccacact |
| | | aagatccacacaggggaaaagccttttcaatgtagaatctgtat |
| | | gagaaactttagtacatccggaaatctcacacgacatatcagaa |
| | | cccacactggagaaaaaccttttgcctgcgacatctgcggaaga |
| | | aaattcgcccgaaggtcccacttgactagtcataccaaaatcca |
| | | cttgcgaggctcacagctggttaaatccgaacttgaagaaaaaa |
| | | aaagtgaactgcggcataaactgaagtatgtcccccatgaatat |
| | | atcgaactgatagaaatcgcccgaaatagcacccaagatagaat |
| | | cctcgaaatgaaggttatggaatttttcatgaaggtctatggat |
| | | ataggggcaagcaccttggcggatcccggaaacctgatggagct |
| | | atctacacagtgggctcaccaatagactatggagttatcgtcga |
| | | tacaaaagcatacagcggaggatacaatttgccaataggtcaag |
| | | cagatgagatggaaagatacgtggaggaaaaccaaacaagagat |
| | | aagcatctgaaccccaacgaatggtggaaagtgtaccccagttc |
| | | tgtaaccgaatttaagttcttgttcgtttcaggtcacttcaagg |
| | | gtaattacaaggctcaactgactagactcaaccatattacaaat |
| | | tgcgatggtgctgtgctttccgtggaagaattgctgattggtgg |
| | | agagatgataaaagctggtaccctcaccttggaagaagtgcgca |
| | | gaaaattcaataatggcgagatcaacttccgaagt(GGCAGCGG |
| | | AGAGGGCAGAGGAAGCCTGCTCACCTGCGGTGACGTGGAGGAAA |
| | | 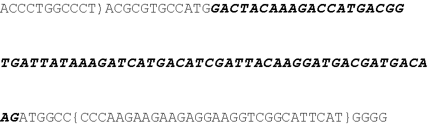 |
| | | 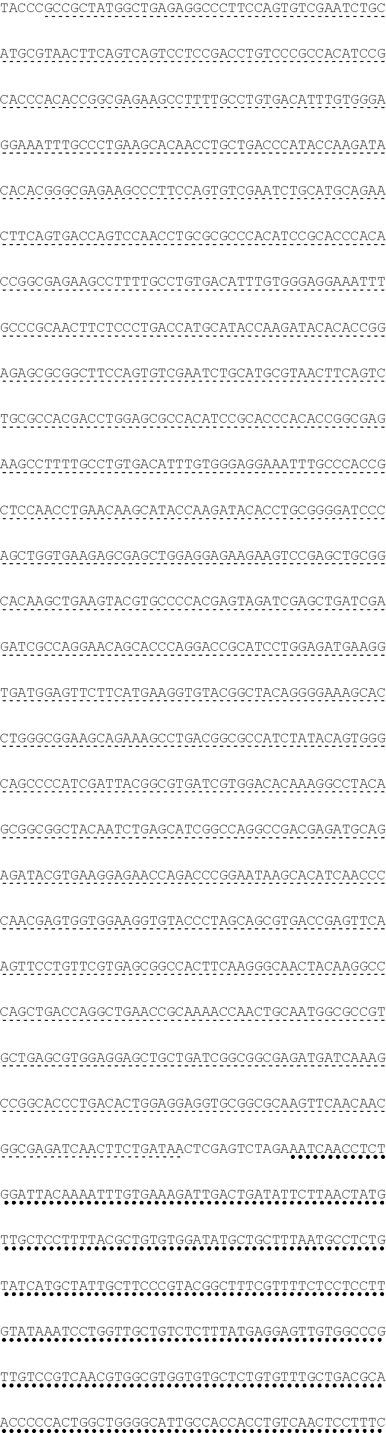 |
| | | 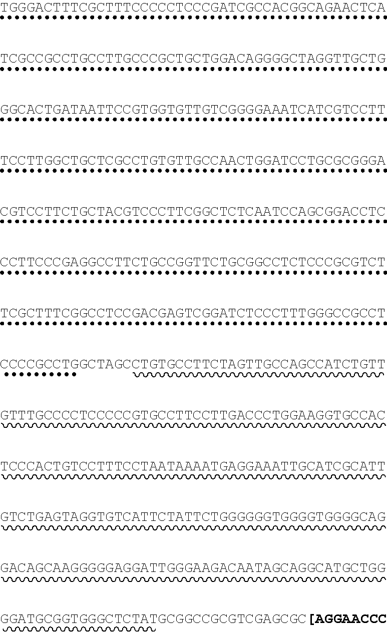 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 44 | GUS144- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (L2_HL-R) | GCGGCCTAAGCTTGAGCTCTTCGAAAGGCTCAGAGGCACACAGG |
| | (na) | AGTTTCTGGGCTCACCCTGCCCCCTTCCAACCCCTCAGTTCCCA |
| | ZFN-L | TCCTCCAGCAGCTGTTTGTGTGCTGCCTCTGAAGTCCACACTGA |
| | Codon | ACAAACTTCAGCCTAGTCATGTCCCTAAAATGGGCAAACATTGC |
| | diversified | AAGCAGCAAACAGCAAACACACAGCCCTCCCTGCCTGCTGACCT |
| | Version 5 | TGGAGCTGGGGCAGAGGTCAGAGACCTCTCTGGGCCCATGCCAC |
| | ZFN-R | CTCCAACATCCACTCGACCCCTTGGAATTTCGGTGGAGAGGAGC |
| | Not | AGAGGTTGTCCTGGCGTGGTTTAGGTAGTGTGAGAGGGGTCCCG |
| | diversified | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 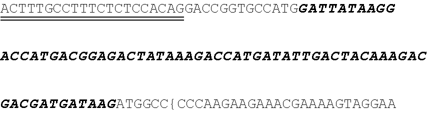 |
| | | TCCAT}GGCGTGCCTgcagcaatggcagagagaccatttcagtg |
| | | cagaatatgtatgcaaaacttctcccagagcggtaatctggcta |
| | | ggcatattagaacacacaccggggaaaaacctttcgcttgcgat |
| | | atatgtggtagaaagttcgccctcaaacagaatctgtgcatgca |
| | | cactaaaatccatacaggagaaaagccctttcagtgtagaattt |
| | | gtatgcagaaatttgcttggcagtcaaatttgcaaaatcacacc |
| | | aaaatacacacaggagaaaaaccatttcagtgtagaatatgtat |
| | | gagaaatttttccacttccggaaatctgaccagacatatacgga |
| | | cacacactggggaaaagcccttcgcttgcgacatctgcggaaga |
| | | aagttcgctagacggtcccacttgacatcccacactaagataca |
| | | tcttcgcggtagccaactggtgaaaagtgaactggaggaaaaaa |
| | | aatctgagctgagacataaactgaaatacgtaccacatgaatac |
| | | atagaacttatagaaatagctaggaactccacccaggacagaat |
| | | acttgaaatgaaggtcatggagttttttatgaaagtttacggat |
| | | acaggggcaaacaccttggagggtctcggaagcctgatggcgca |
| | | atttataccgtgggtagccctatagattatggagtgattgtgga |
| | | tacaaaggcttacagtggcggctataatttgcctatcggacagg |
| | | ccgatgagatggaaagatacgttgaagaaaaccaaacacgagat |
| | | aagcatctgaaccccaatgaatggtggaaagtgtatccttcaag |
| | | cgttaccgagtttaagttcctcttcgtttctgggcatttcaagg |
| | | gcaactacaaagctcagcttacaagactcaaccacataaccaat |
| | | tgtgatggagcagtcctcagcgtggaagaactccttattggggg |
| | | tgagatgattaaagcagggacccttactcttgaagaggttagaa |
| | | gaaaattcaataacggagagattaattttagaagt(GGCAGCGG |
| | | AGAGGGCAGAGGAAGCCTGCTCACCTGCGGTGACGTGGAGGAAA |
| | | 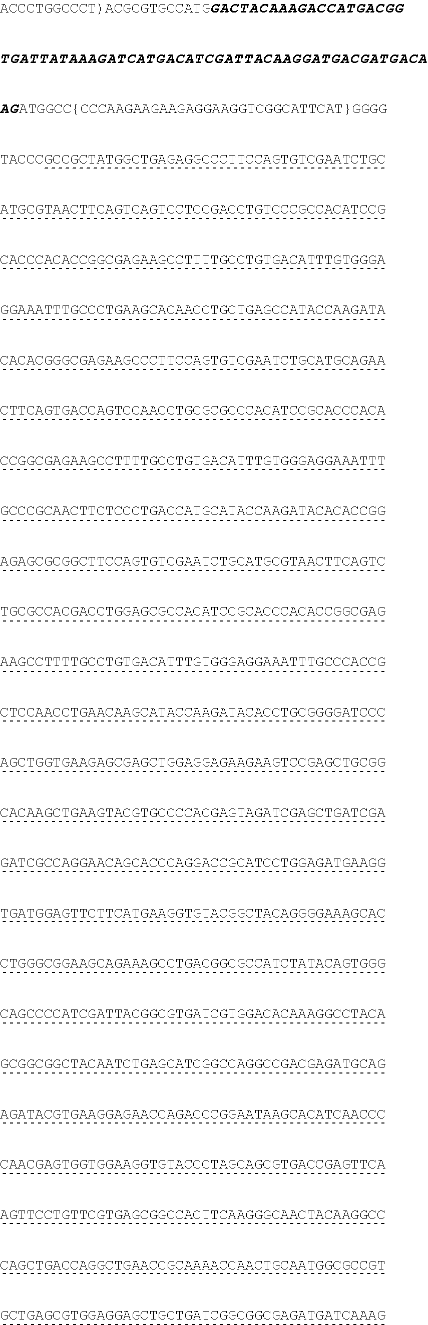 |
| | | 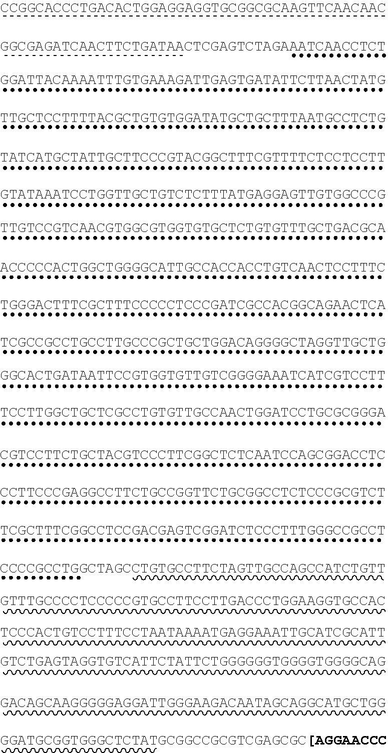 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 45 | GUS145- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (L3_HL-R) | GCGGCCTAAGCTTGAGCTCTTCGAAAGGCTCAGAGGCACACAGG |
| | (na) | AGTTTCTGGGCTCACCCTGCCCCCTTCCAACCCCTCAGTTCCCA |
| | ZFN-L | TCCTCCAGCAGCTGTTTGTGTGCTGCCTCTGAAGTCCACACTGA |
| | Codon | ACAAACTTCAGCCTAGTCATGTCCCTAAAATGGGCAAACATTGC |
| | diversified | AAGCAGCAAACAGCAAACACACAGCCCTCCCTGCCTGCTGACCT |
| | Version 6 | TGGAGCTGGGGCAGAGGTCAGAGACCTCTCTGGGCCCATGCCAC |
| | ZFN-R | CTCCAACATCCACTCGACCCCTTGGAATTTCGGTGGAGAGGAGC |
| | Not | AGAGGTTGTCCTGGCGTGGTTTAGGTAGTGTGAGAGGGGTCCCG |
| | diversified | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 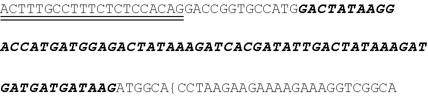 |
| | | TTCAT}GGTGTGCCTgcagccatggccgaacgcccatttcaatg |
| | | tagaatttgtatgcagaatttttcacaatcaggaaacctggcta |
| | | gacatatcagaacacatactggagaaaagccctttgcttgtgat |
| | | atctgtggaaggaaattcgccctgaaacaaaacctctgtatgca |
| | | cacaaagatccacaccggcgaaaagcctttccagtgtaggatat |
| | | gcatgcaaaaattcgcctggcagtccaatctgcagaaccatacc |
| | | aaaattcatactggtgaaaagccatttcagtgcagaatatgtat |
| | | gagaaactttagcacttcaggaaatctcacaagacatataagaa |
| | | cacatacaggggaaaaaccttttgcttgcgatatctgcggcagg |
| | | aaattcgctcggagaagtcatctcacaagccatacaaaaatcca |
| | | cctgcgaggaagccagctggtcaagtctgaactggaagaaaaaa |
| | | aaagcgaactgcggcataaactcaaatacgtcccacatgaatac |
| | | attgagctcatcgaaattgctagaaactctactcaagataggat |
| | | attggagatgaaggtaatggaattcttcatgaaggtttatggat |
| | | atagaggaaaacatcttggaggcagtaggaaacccgatggcgct |
| | | atctacaccgtagggagtccaatcgactacggcgtgattgttga |
| | | caccaaagcctattctggagggtataatctcccaattggacagg |
| | | cagatgagatggaaagatatgtagaagaaaatcagacaagagat |
| | | aagcaccttaaccctaacgagtggtggaaagtgtacccaagcag |
| | | tgttactgaatttaaatttctttttgtatcaggacactttaaag |
| | | gcaattacaaagcacaactgaccagactcaatcacattaccaat |
| | | tgcgacggagccgtactgagcgtggaggagttgctgatcggagg |
| | | cgaaatgattaaagctggcactctgaccctggaagaagtaagaa |
| | | gaaagttcaataatggagaaataaactttcgctcc(GGCAGCGG |
| | | AGAGGGCAGAGGAAGCCTGCTCACCTGCGGTGACGTGGAGGAAA |
| | | 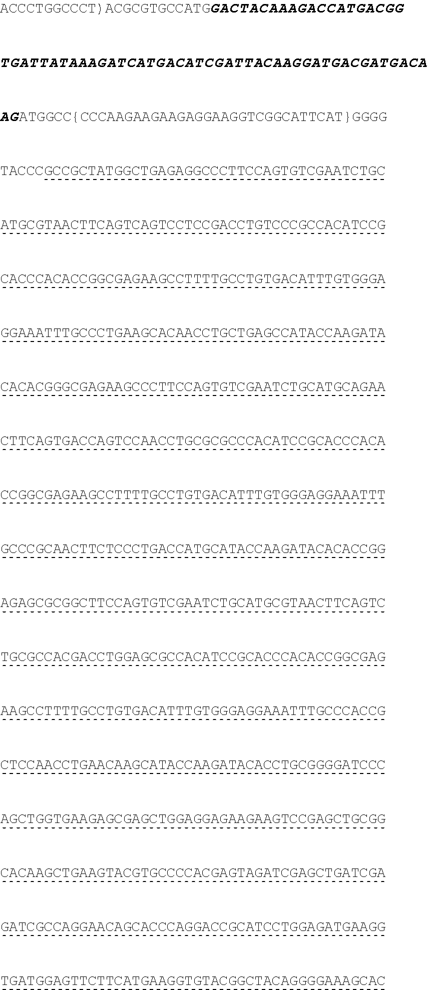 |
| | | 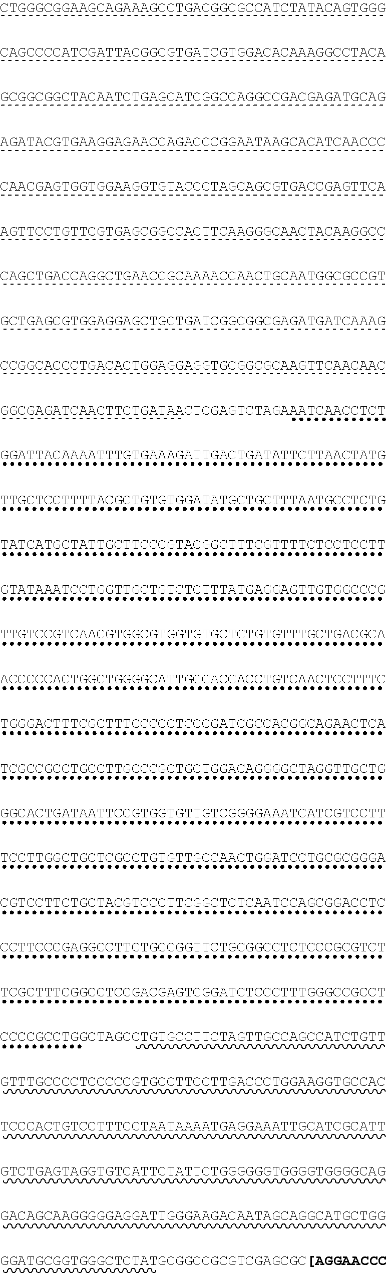 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 46 | GUS146- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (L-R)(na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Not | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | ZFN-L | AAGCAGCAAACAGCAAACACACAGCCCTCCCTGCCTGCTGACCT |
| | Not | TGGAGCTGGGGCAGAGGTCAGAGACCTCTCTGGGCCCATGCCAC |
| | diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 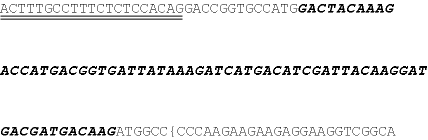 |
| | | TTCAT}GGGGTACCCgccgctatggctgagaggcccttccagtg |
| | | tcgaatctgcatgcagaacttcagtcagtccggcaacctggccc |
| | | gccacatccgcacccacaccggcgagaagccttttgcctgtgac |
| | | atttgtgggaggaaatttgccctgaagcagaacctgtgtatgca |
| | | taccaagatacacacgggcgagaagcccttccagtgtcgaatct |
| | | gcatgcagaagtttgcctggcagtccaacctgcagaaccatacc |
| | | aagatacacacgggcgagaagcccttccagtgtcgaatctgcat |
| | | gcgtaacttcagtacctccggcaacctgacccgccacatccgca |
| | | cccacaccggcgagaagccttttgcctgtgacatttgtgggagg |
| | | aaatttgcccgccgctcccacctgacctcccataccaagataca |
| | | cctgcggggatcccagctggtgaagagcgagctggaggagaaga |
| | | agtccgagctgcggcacaagctgaagtacgtgccccacgagtac |
| | | atcgagctgatcgagatcgccaggaacagcacccaggaccgcat |
| | | cctggagatgaaggtgatggagttcttcatgaaggtgtacggct |
| | | acaggggaaagcacctgggcggaagcagaaagcctgacggcgcc |
| | | atctatacagtgggcagccccatcgattacggcgtgatcgtgga |
| | | cacaaaggcctacagcggcggctacaatctgcctatcggccagg |
| | | ccgacgagatggagagatacgtggaggagaaccagacccgggat |
| | | aagcacctcaaccccaacgagtggtggaaggtgtaccctagcag |
| | | cgtgaccgagttcaagttcctgttcgtgagcggccacttcaagg |
| | | gcaactacaaggcccagctgaccaggctgaaccacatcaccaac |
| | | tgcgacggcgccgtgctgagcgtggaggagctgctgatcggcgg |
| | | cgagatgatcaaagccggcaccctgacactggaggaggtgcggc |
| | | gcaagttcaacaacggcgagatcaacttcagatct(GGCAGCGG |
| | | AGAGGGCAGAGGAAGCCTGCTCACCTGCGGTGACGTGGAGGAAA |
| | | 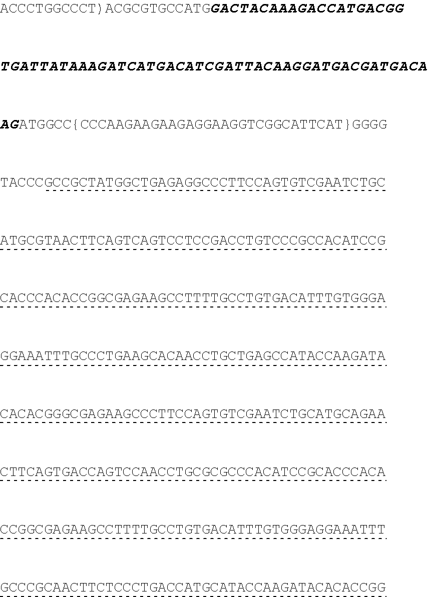 |
| | | 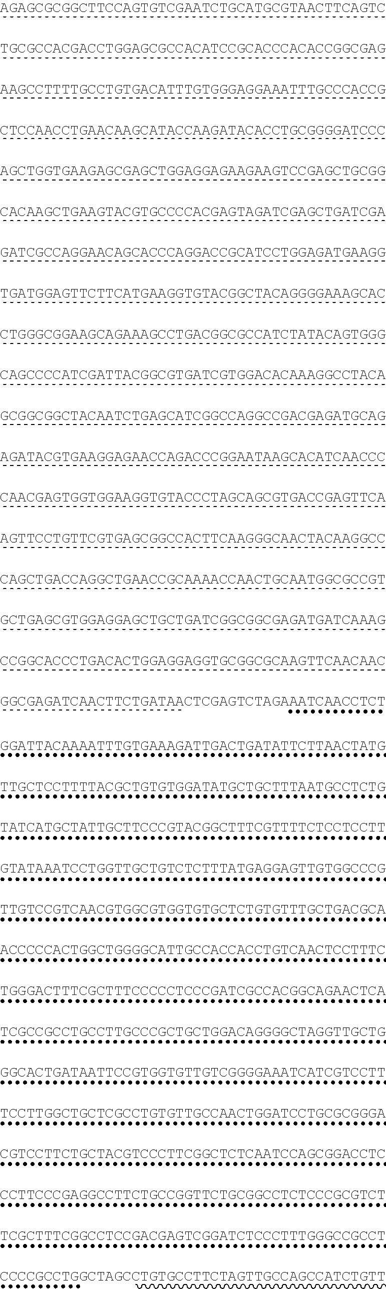 |
| | | 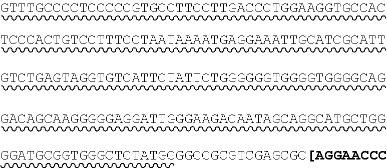 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 47 | GUS150- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (L2-R1_HL) | GCGGCCTAAGCTTGAGCTCTTCGAAAGGCTCAGAGGCACACAGG |
| | (na) | AGTTTCTGGGCTCACCCTGCCCCCTTCCAACCCCTCAGTTCCCA |
| | ZFN-L | TCCTCCAGCAGCTGTTTGTGTGCTGCCTCTGAAGTCCACACTGA |
| | Codon | ACAAACTTCAGCCTAGTCATGTCCCTAAAATGGGCAAACATTGC |
| | diversified | AAGCAGCAAACAGCAAACACACAGCCCTCCCTGCCTGCTGACCT |
| | Version 2 | TGGAGCTGGGGCAGAGGTCAGAGACCTCTCTGGGCCCATGCCAC |
| | ZFN-R | CTCCAACATCCACTCGACCCCTTGGAATTTCGGTGGAGAGGAGC |
| | Codon | AGAGGTTGTCCTGGCGTGGTTTAGGTAGTGTGAGAGGGGTCCCG |
| | diversified | GGGATCTTGCTACCAGTGGAACAGCCACTAAGGATTCTGCAGTG |
| | Version 4 | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 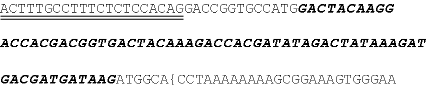 |
| | | TTCAC}GGCGTGCCCgccgccatggcagagagaccctttcaatg |
| | | tagaatctgtatgcaaaatttctctcagagtggtaaccttgcaa |
| | | gacacatcagaactcatacaggtgagaagccgtttgcatgtgac |
| | | atttgcggtaggaaatttgccttgaaacagaatctttgtatgca |
| | | cacaaaaatccatactggtgaaaagccattccaatgccgcatct |
| | | gtatgcaaaaattcgcgtggcagtccaatttgcagaaccatacc |
| | | aagattcacacgggagaaaaaccatttcagtgccgcatctgcat |
| | | gcgcaacttttctacatcaggaaaccttacacgacatattcgga |
| | | cgcacactggagaaaaaccatttgcttgtgacatatgcggccga |
| | | aaatttgccagacgctctcatctcacctcacatactaagattca |
| | | tttgcgcggaagtcagctggtgaagagtgaattggaagaaaaaa |
| | | agtcagagctgagacacaaactgaaatatgttccacacgagtac |
| | | atcgagcttatcgagatagcaagaaactccacccaggacagaat |
| | | tttggaaatgaaagttatggaattctttatgaaagtgtatggct |
| | | acaggggtaaacatctggggggatcaagaaagcctgatggtgca |
| | | atttacacagtgggctctcctatcgactacggtgtgatcgtgga |
| | | tacaaaggcctactctggaggatataatttgcctattggacaag |
| | | ccgatgaaatggaaagatatgtggaggaaaaccagactcgcgat |
| | | aagcacctgaacccaaatgaatggtggaaagtgtacccttcatc |
| | | tgttaccgaatttaaatttttgttcgtttccgggcatttcaagg |
| | | ggaactacaaggcacagctgacgagactgaatcacatcacgaac |
| | | tgcgacggcgctgtactgtccgtggaagagcttttgatcggggg |
| | | cgaaatgattaaggccggcacactgacgctggaggaggtgcggc |
| | | gaaaatttaataatggcgagatcaattttaggagt(GGCAGCGG |
| | | AGAGGGCAGAGGAAGCCTGCTCACCTGCGGTGACGTGGAGGAAA |
| | | 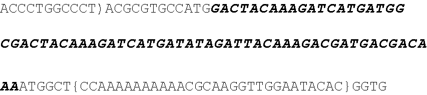 |
| | | 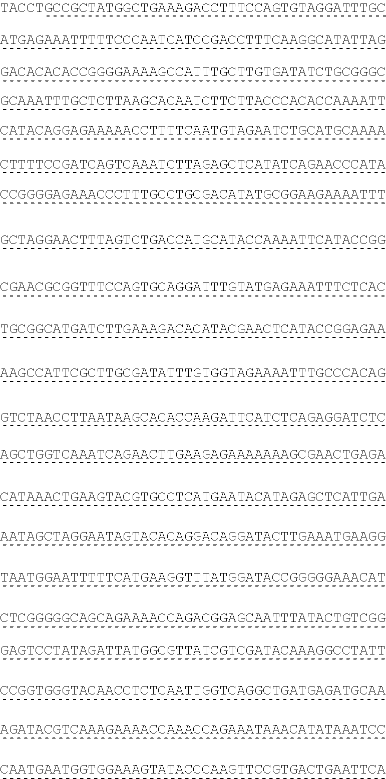 |
| | | 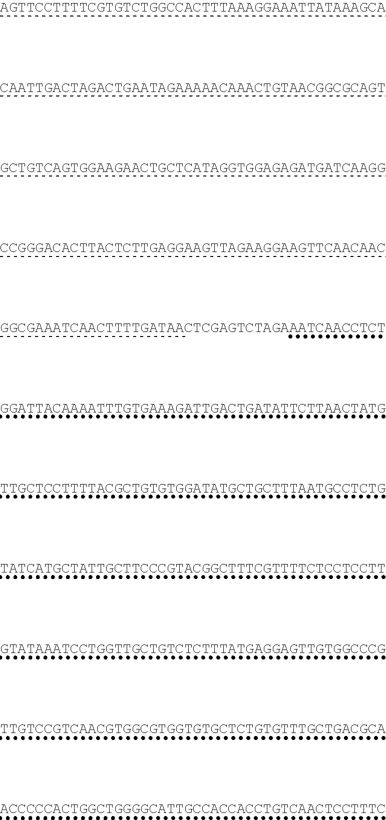 |
| | | 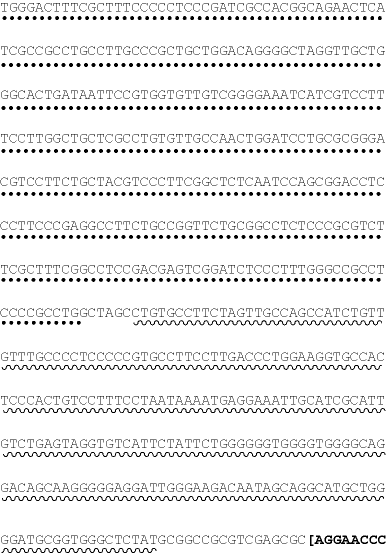 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 48 | GUS151- | [CTGCGCGCTCGCTCGCTCACTGAGGCCGCCCGGGCAAAGCCCG |
| | pAAV-hZFN- | GGCGTCGGGCGACCTTTGGTCGCCCGGCCTCAGTGAGCGAGCGA |
| | 2-in-1 vector | GCGCGCAGAGAGGGAGTGGCCAACTCCATCACTAGGGGTTCCT] |
| | (R1-_HL-L2) | GCGGCCTAAGCTTGAGCTCTTCGAAAGGCTCAGAGGCACACAGG |
| | (na) | AGTTTCTGGGCTCACCCTGCCCCCTTCCAACCCCTCAGTTCCCA |
| | ZFN-R | TCCTCCAGCAGCTGTTTGTGTGCTGCCTCTGAAGTCCACACTGA |
| | Codon | ACAAACTTCAGCCTAGTCATGTCCCTAAAATGGGCAAACATTGC |
| | diversified | AAGCAGCAAACAGCAAACACACAGCCCTCCCTGCCTGCTGACCT |
| | Version 4 | TGGAGCTGGGGCAGAGGTCAGAGACCTCTCTGGGCCCATGCCAC |
| | ZFN-L | CTCCAACATCCACTCGACCCCTTGGAATTTCGGTGGAGAGGAGC |
| | Codon | AGAGGTTGTCCTGGCGTGGTTTAGGTAGTGTGAGAGGGGTCCCG |
| | diversified | GGGATCTTGCTACCAGTGGAACAGCCACTAAGGATTCTGCAGTG |
| | Version 2 | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | 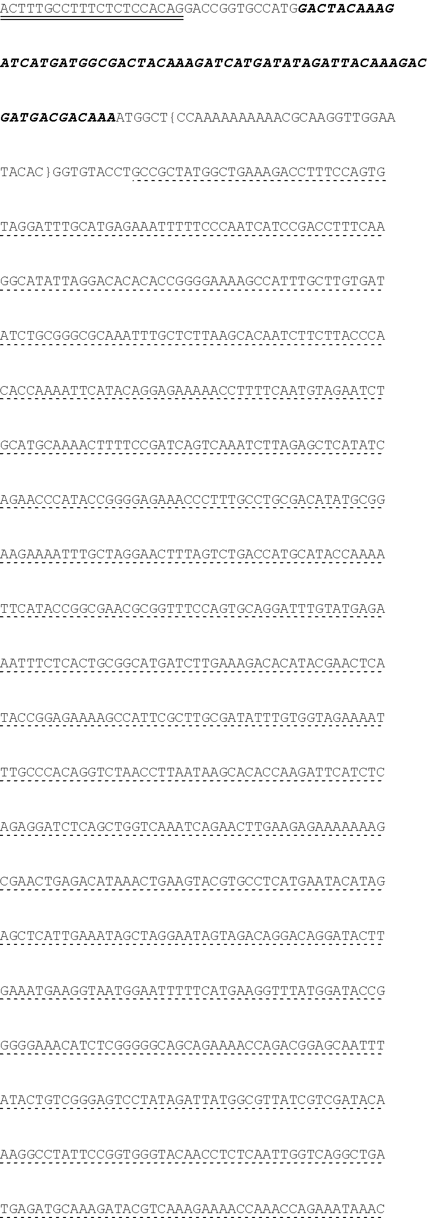 |
| | | 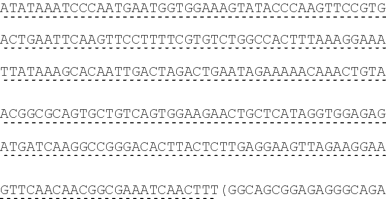 |
| | | GGAAGCCTGCTCACCTGCGGTGACGTGGAGGAAAACCCTGGCCC |
| | | 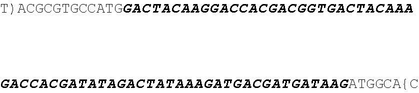 |
| | | CTAAAAAAAAGCGGAAAGTGGGAATTCAC}GGCGTGCCCgccgc |
| | | catggcagagagaccctttcaatgtagaatctgtatgcaaaatt |
| | | tctctcagagtggtaaccttgcaagacacatcagaactcataca |
| | | ggtgagaagccgtttgcatgtgacatttgcggtaggaaatttgc |
| | | cttgaaacagaatctttgtatgcacacaaaaatccatactggtg |
| | | aaaagccattccaatgccgcatctgtatgcaaaaattcgcgtgg |
| | | cagtccaatttgcagaaccataccaagattcacacgggagaaaa |
| | | accatttcagtgccgcatctgcatgcgcaacttttctacatcag |
| | | gaaaccttacacgacatattcggacgcacactggagaaaaacca |
| | | tttgcttgtgacatatgcggccgaaaatttgccagacgctctca |
| | | tctcacctcacatactaagattcatttgcgcggaagtcagctgg |
| | | tgaagagtgaattggaagaaaaaaagtcagagctgagacacaaa |
| | | ctgaaatatgttccacacgagtacatcgagcttatcgagatagc |
| | | aagaaactccacccaggacagaattttggaaatgaaagttatgg |
| | | aattctttatgaaagtgtatggctacaggggtaaacatctgggg |
| | | ggatcaagaaagcctgatggtgcaatttacacagtgggctctcc |
| | | tatcgactacggtgtgatcgtggatacaaaggcctactctggag |
| | | gatataatttgcctattggacaagccgatgaaatggaaagatat |
| | | gtggaggaaaaccagactcgcgataagcacctgaacccaaatga |
| | | atggtggaaagtgtacccttcatctgttaccgaatttaaatttt |
| | | tgttcgtttccgggcatttcaaggggaactacaaggcacagctg |
| | | acgagactgaatcacatcacgaactgcgacggcgctgtactgtc |
| | | cgtggaagagcttttgatcgggggcgaaatgattaaggccggca |
| | | cactgacgctggaggaggtgcggcgaaaatttaataatggcgag |
| | | 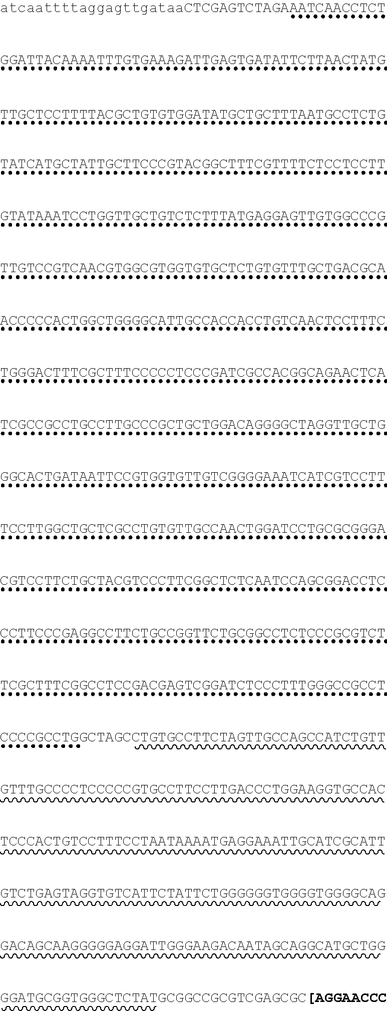 |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |

Legend:
5′ITR = [plain text in brackets]
ApoE (Enhancer) = underline
hAAT (Promoter) = italics
5′UTR = bold
Human β-globin/IgG chimeric intron = double underline
3xFLAG = bold italics
NLS = {plain text in curly brackets}
ZFN-L = lower case
2A peptide = (plain text in parentheses)
ZFN-R = Dashed underline
WPREmut6 = Dotted underline
Polyadenylation signal = Wavy underline
3′ITR = [bold text in brackets]

The following Examples relate to exemplary embodiments of the present disclosure in which the donor comprises a push-pull donor polynucleotide and nuclease comprises a zinc finger nuclease (ZFN). It will be appreciated that these examples are included merely for the purpose of illustration of certain features and embodiments of the present disclosure and are not intended to be limiting. Those skilled in the art will also recognize, or be able to ascertain using no more than routine experimentation numerous equivalents to the methods, nucleic acids, proteins, vectors and cells described herein. Such equivalents are considered to be within the scope of the present disclosure.

### NUMBERED EMBODIMENTS

Particular embodiments of the disclosure are set forth in the following numbered paragraphs:

1. A polynucleotide construct comprising in 5′ to 3′ orientation:
a. a first Inverted Terminal Repeat (ITR) nucleotide sequence;
b. a first nucleotide sequence encoding a first polypeptide;
c. a second nucleotide sequence encoding a second polypeptide; and
d. a second ITR nucleotide sequence;
   wherein the first nucleotide sequence encoding a first polypeptide is oriented tail-to-tail to the second nucleotide sequence encoding a second polypeptide; and wherein the first nucleotide sequence and the second nucleotide sequence encode a polypeptide having the same amino acid sequence.
   2. The polynucleotide construct according to paragraph 1, further comprising:
e. a first splice acceptor sequence operatively linked to the first nucleotide sequence encoding the first polypeptide; and
f. a second splice acceptor sequence operatively linked to the second nucleotide sequence encoding the second polypeptide.
   3. The polynucleotide construct according to paragraph 2, wherein each of said first splice acceptor sequence and second splice acceptor sequence is independently selected from a Factor 9 Splice Acceptor (F9SA), a CFTR Splice acceptor, a COL5A2 Splice acceptor, a NF1 Splice Acceptor, a MLH1 Splice Acceptor, and an Albumin (ALB) Splice Acceptor.
   4. The polynucleotide construct according to paragraph 1 or 2, further comprising:
g. a first polyadenylation (polyA) signal sequence operatively linked to the nucleotide sequence encoding the first polypeptide; and
h. a second polyadenylation (polyA) signal sequence operatively linked to the nucleotide sequence encoding the second polypeptide.
   5. The polynucleotide construct according to paragraph 4, wherein the first polyA signal sequence is selected from a human Growth Hormone (hGH) polyA signal, a bovine Growth Hormone (bGH) polyA signal, a SV40 polyA signal, and a rbGlob polyA signal.
   6. The polynucleotide construct according to paragraph 4 or 5, wherein the second polyA signal sequence is selected from a human Growth Hormone (hGH) polyA signal, a bovine Growth Hormone (bGH) polyA signal, a SV40 polyA signal, and a rbGlob polyA signal.
   7. The polynucleotide construct according to any one of paragraphs 1-6, wherein the nucleotide sequence encoding the first polypeptide or the nucleotide sequence encoding the second polypeptide encodes a therapeutic polypeptide.
   8. The polynucleotide construct according to paragraph 7, wherein the therapeutic polypeptide is selected from the group consisting of iduronate-2-sulphatase (IDS), alpha-L-iduronidase (IDUA), alpha-D-mannosidase, N-aspartyl-beta-glucosaminidase, lysosomal acid lipase, cystinosin, lysosomal associated membrane protein 2, alpha-galactosidase A, acid ceramidase, alpha fucosidase, cathepsin A, acid beta-glucocerebrosidase, beta galactosidase, beta hexosaminidase A, beta hexosaminidase B, beta hexosaminidase, GM2 ganglioside activator, GLcNAc-1-phosphotransferase, Beta-galactosylceramidase, arylsulfatase A, heparan N-sulfatase, alpha-N-acetylglucosaminidase, acetyl CoA:alpha-glucosaminide acetyltransferase, N-acetyl glucosamine-6-sulfatase, arylsulfatase B, beta-glucuronidase, hyaluronidase, neuraminidase, mucolipin-1, formylglycine-generating enzyme, palmitoyl-protein thioesterase 1, tripeptidyl peptidase 1, CLN3 protein, cysteine string protein alpha, CLN5 protein, CLN6 protein, CLN7 protein, CLN8 protein, acid sphingomyelinase, NPC 1, NPC 2, phenylalanine hydroxylase, acid alpha-glucosidase, cathepsin K, sialin, alpha-N-acetylgalactosaminidase, glucose-6-phosphatase, solute carrier family 37 member 4, argininosuccinate synthase 1, solute carrier family 25 member 13, and ornithine transcarbamylase.
   9. The polynucleotide construct according to any one of paragraphs 1-8, wherein the nucleotide sequence encoding the first polypeptide is codon diversified.
   10. The polynucleotide construct according to any one of paragraphs 1-9, wherein the nucleotide sequence encoding the second polypeptide is codon diversified.
   11. The polynucleotide construct according to any one of paragraphs 1-10, wherein each of the nucleotide sequence encoding the first polypeptide and the nucleotide sequence encoding the second polypeptide is each independently codon diversified.
   12. The polynucleotide construct according to any one of paragraphs 1-11, wherein the nucleotide sequence encoding the first polypeptide comprises the nucleotide sequence set forth in any one of SEQ ID NOs: 184-193.
   13. The polynucleotide construct according to any one of paragraphs 1-12, wherein the nucleotide sequence encoding the second polypeptide comprises the nucleotide sequence set forth in any one of SEQ ID NOs: 184-193.
   14. The polynucleotide construct according to paragraph 1, wherein said polynucleotide construct comprises the nucleotide sequence set forth in any one of SEQ ID NOs: 173-176.
   15. A vector comprising the polynucleotide construct according to any one of paragraphs 1-14.
   16. The vector according to paragraph 15, wherein the vector is an adeno-associated viral (AAV) vector.
   17. The vector according to paragraph 16, wherein the AAV is selected from the group consisting of AAV-MeCP2, AAV1, AAV2, AAV3, AAV4, AAV5, AAV6, AAV8, AAV8.2, AAV9, Dual AAV9, AAVrh8, AAVrh10, AAHrh43, AAVhu37, AAV2/8, AAV2/5, and AAV2/6.
   18. A cell comprising the polynucleotide construct according to any one of paragraphs 1-14 or the vector according to any one of paragraphs 15-17.
   19. The cell according to paragraph 18, wherein the cell is a eukaryotic cell.
   20. The cell according to paragraph 19, wherein the cell is a mammalian cell.
   21. The cell according to paragraph 20, wherein the cell is a stem cell.
   22. The cell according to paragraph 19, wherein the cell is a human cell.
   23. The cell according to any one of paragraphs 18-22, wherein the cell is a non-dividing cell.
   24. The cell according to any one of paragraphs 19-23, wherein the cell is a hepatocyte.
   25. The cell according to any one of paragraphs 18-24, wherein the cell further comprises a polynucleotide encoding a nuclease.
   26. The cell according to any one of paragraphs 18-24, wherein the cell further comprises a first polynucleotide encoding a first zinc finger nuclease (ZFN) and a second polynucleotide encoding a second zinc finger nuclease (ZFN).
   27. The cell according to any one of paragraphs 18-24, wherein the cell further comprises a first vector comprising a first polynucleotide encoding a first zinc finger nuclease (ZFN) and a second vector comprising a second polynucleotide encoding a second zinc finger nuclease (ZFN).
   28. The cell according to any one of paragraphs 18-24, wherein the cell further comprises a polynucleotide encoding one or more zinc finger nucleases (ZFN).
   29. The cell according to any one of paragraphs 18-24, wherein the cell further comprises a vector comprising a polynucleotide encoding one or more zinc finger nucleases (ZFN).
   30. The cell according to paragraph 28 or 29, wherein the zinc finger nuclease is a 2-in-1 zinc finger nuclease.
   31. A pharmaceutical composition comprising the polynucleotide construct according to any one of paragraphs 1-14; and a pharmaceutically acceptable carrier.
   32. The pharmaceutical composition according to paragraph 31, wherein the composition further comprises a first polynucleotide encoding a first zinc finger nuclease (ZFN) and a second polynucleotide encoding a second zinc finger nuclease (ZFN).
   33. The pharmaceutical composition according to paragraph 31, wherein the composition comprises a first vector comprising a first polynucleotide encoding a first zinc finger nuclease (ZFN) and a second vector comprising a second polynucleotide encoding a second zinc finger nuclease (ZFN).
   34. The pharmaceutical composition according to paragraph 31, wherein the composition further comprises a polynucleotide encoding one or more zinc finger nucleases (ZFN).
   35. The pharmaceutical composition according to paragraph 32, wherein the composition further comprises a vector comprising a polynucleotide encoding one or more zinc finger nucleases (ZFN).
   36. The pharmaceutical composition according to paragraphs 32, 34-35, wherein the zinc finger nuclease is a 2-in-1 zinc finger nuclease.
   37. The pharmaceutical composition according to paragraph 32, wherein the ratio of the polynucleotide encoding the first zinc finger nuclease: the polynucleotide encoding the second zinc finger: the polynucleotide of any one of paragraphs 1-14 is 1:1:8.
   38. The pharmaceutical composition according to paragraph 32, wherein the ratio of the polynucleotide encoding the first zinc finger nuclease: the polynucleotide encoding the second zinc finger: the polynucleotide of any one of paragraphs 1-14 is 1:1:4.
   39. The pharmaceutical composition according to paragraph 32, wherein the ratio of the polynucleotide encoding the first zinc finger nuclease: the polynucleotide encoding the second zinc finger: the polynucleotide of any one of paragraphs 1-14 is 1:1:2.
   40. The pharmaceutical composition according to paragraph 32, wherein the ratio of the polynucleotide encoding the first zinc finger nuclease: the polynucleotide encoding the second zinc finger: the polynucleotide of any one of paragraphs 1-14 is 3:3:4.
   41. The pharmaceutical composition according to paragraph 33, wherein the ratio of the vector comprising the first polynucleotide encoding the first zinc finger nuclease: the vector comprising the polynucleotide encoding the second zinc finger: the vector of any one of paragraphs 15-17 is 1:1:8.
   42. The pharmaceutical composition according to paragraph 33, wherein the ratio of the vector comprising the first polynucleotide encoding the first zinc finger nuclease: the vector comprising the polynucleotide encoding the second zinc finger: the vector of any one of paragraphs 15-17 is 1:1:4.
   43. The pharmaceutical composition according to paragraph 33, wherein the ratio of the vector comprising the first polynucleotide encoding the first zinc finger nuclease: the vector comprising the polynucleotide encoding the second zinc finger: the vector of any one of paragraphs 15-17 is 1:1:2.
   44. The pharmaceutical composition according to paragraph 33, wherein the ratio of the vector comprising the first polynucleotide encoding the first zinc finger nuclease: the vector comprising the polynucleotide encoding the second zinc finger: the vector of any one of paragraphs 15-17 is 3:3:4.
   45. The pharmaceutical composition according to paragraph 36, wherein the ratio of the polynucleotide encoding the 2-in-1 zinc finger nuclease: the polynucleotide construct of any one of paragraphs 1-14 is 1:4.
   46. The pharmaceutical composition according to paragraph 36, wherein the ratio of the polynucleotide encoding the 2-in-1 zinc finger nuclease: the polynucleotide construct of any one of paragraphs 1-14 is 1:2.
   47. The pharmaceutical composition according to paragraph 36, wherein the ratio of the polynucleotide encoding the 2-in-1 zinc finger nuclease: the polynucleotide construct of any one of paragraphs 1-14 is 1:1.
   48. The pharmaceutical composition according to paragraph 36, wherein the ratio of the polynucleotide encoding the 2-in-1 zinc finger nuclease: the polynucleotide construct of any one of paragraphs 1-14 is 3:2.
   49. The pharmaceutical composition according to paragraph 33, wherein the ratio of the vector comprising the 2-in-1 zinc finger nuclease: the vector of any one of paragraphs 15-17 is 1:4.
   50. The pharmaceutical composition according to paragraph 33, wherein the ratio of the vector comprising the 2-in-1 zinc finger nuclease: the vector of any one of paragraphs 15-17 is 1:2.
   51. The pharmaceutical composition according to paragraph 33, wherein the ratio of the vector comprising the 2-in-1 zinc finger nuclease: the vector of any one of paragraphs 15-17 is 1:1.
   52. The pharmaceutical composition according to paragraph 33, wherein the ratio of the vector comprising the 2-in-1 zinc finger nuclease: the vector of any one of paragraphs 15-17 is 3:2.
   53. The pharmaceutical composition of any one of paragraphs 31-53, wherein the composition is formulated for intravenous, intramuscular, subcutaneous, or intrathecal administration.
   54. A method for modifying the genome of a cell, the method comprising introducing into a cell an effective amount of the polynucleotide construct according to any one of paragraphs 1-14.
   55. A method for modifying the genome of a cell, the method comprising introducing into a cell an effective amount of the vector according to any one of paragraphs 15-17.
   56. A method for modifying the genome of a cell, the method comprising introducing into a cell an effective amount of the pharmaceutical composition according to any one of paragraphs 31-53.
   57. A method for integrating an exogenous nucleotide sequence into a target nucleotide sequence of a cell, the method comprising introducing into a cell an effective amount of the polynucleotide construct according to any one of paragraphs 1-14.
   58. A method for integrating an exogenous nucleotide sequence into a target nucleotide sequence of a cell, the method comprising introducing into a cell an effective amount of the vector according to any one of paragraphs 15-17.
   59. A method for integrating an exogenous nucleotide sequence into a target nucleotide sequence of a cell, the method comprising introducing into a cell an effective amount of the pharmaceutical composition according to any one of paragraphs 31-53.
   60. A method for disrupting a target nucleotide sequence in a cell, the method comprising introducing into a cell an effective amount of the polynucleotide construct according to any one of paragraphs 1-14.
   61. A method for disrupting a target nucleotide sequence in a cell, the method comprising introducing into a cell an effective amount of the vector according to any one of paragraphs 15-17.
   62. A method for disrupting a target nucleotide sequence in a cell, the method comprising introducing into a cell an effective amount of the pharmaceutical composition according to any one of paragraphs 31-53.
   63. A method for treating a disorder in a subject, the method comprising modifying a target nucleotide sequence in the genome of a cell of said subject by introducing into the cell an effective amount of the polynucleotide construct according to any one of paragraphs 1-14.
   64. A method for treating a disorder in a subject, the method comprising modifying a target nucleotide sequence in the genome of a cell of said subject by introducing into the cell an effective amount of the vector according to any one of paragraphs 15-17.
   65. A method for treating a disorder in a subject, the method comprising modifying a target nucleotide sequence in the genome of a cell of said subject by introducing into the cell an effective amount of the pharmaceutical composition according to any one of paragraphs 31-53.
   66. The method according to any one of paragraphs 54, 57, 60 and 63, the method further comprising introducing into the cell an effective amount of a first polynucleotide encoding a first zinc finger nuclease (ZFN) and a second polynucleotide encoding a second zinc finger nuclease (ZFN).
   67. The method according to any one of paragraphs 55, 58, 61 and 64, the method further comprising introducing into the cell an effective amount of a first vector comprising a first polynucleotide encoding a first zinc finger nuclease (ZFN) and a second vector comprising a second polynucleotide encoding a second zinc finger nuclease (ZFN).
   68. The method according to any one of paragraphs 54, 57, 60 and 63, the method further comprising introducing into the cell an effective amount of a polynucleotide encoding one or more zinc finger nucleases (ZFN).
   69. The method according to any one of paragraphs 55, 58, 61 and 64, the method further comprising introducing into the cell an effective amount of a vector comprising a polynucleotide encoding one or more zinc finger nucleases (ZFN).
   70. The method according to paragraph 68-69, wherein the zinc finger nuclease is a 2-in-1 zinc finger nuclease.
   71. The method according to any one of paragraphs 54-70, wherein upon integration of the polynucleotide construct of any one of paragraphs 1-14 into the genome of the cell, the first nucleotide sequence encoding the first polypeptide is expressed.
   72. The method according to any one of paragraphs 54-70, wherein upon integration of the polynucleotide construct of any one of paragraphs 1-14 into the genome of the cell, the second nucleotide sequence encoding the second polypeptide is expressed.
   73. The method according to any one of paragraph 63-72, wherein the disorder is selected from the group consisting of a, a genetic disorder, an infectious disease, an acquired disorder, and a cancer.
   74. The method according to paragraph 73, wherein the genetic disorder is selected from the group consisting of achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangiectasia, barth syndrome, beta-thalassemia, blue rubber bleb nevus syndrome, canavan disease, chronic granulomatous diseases (CGD), citrullinemia, cri du chat syndrome, cystic fibrosis, dercum's disease, ectodermal dysplasia, Fabry disease, fanconi anemia, fibrodysplasia ossificans progressive, fragile X syndrome, galactosemis, Gaucher's disease, generalized gangliosidoses (e.g., GM1), GSD (e.g., GSD1a) hemochromatosis, the hemoglobin C mutation in the 6th codon of beta-globin (HbC), hemophilia, Hunter syndrome, Huntington's disease, Hurler Syndrome, hypophosphatasia, Klinefelter syndrome, Krabbes Disease, Langer-Giedion Syndrome, leukocyte adhesion deficiency (LAD, OMIM No. 116920), leukodystrophy, long QT syndrome, lipoprotein lipase deficiency, Marfan syndrome, Moebius syndrome, mucopolysaccharidosis (MPS), nail patella syndrome, nephrogenic diabetes insipdius, neurofibromatosis, Neimann-Pick disease, ornithine transcarbamylase (OTC) deficiency, osteogenesis imperfecta, phenylketonuria (PKU), Pompe disease, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, and X-linked lymphoproliferative syndrome (XLP, OMIM No. 308240).
   75. The method according to paragraph 73, wherein the genetic disorder is a lysosomal storage disease.
   76. The method according to paragraph 75, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/J/A), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.
   77. The method according to paragraph 76, wherein the lysosomal storage disease is selected from MPSI and MPSII.
   78. The method according to paragraph 77, wherein the lysosomal storage disease is selected from the group consisting of MPS I—Hurler Syndrome, MPS I—Scheie Syndrome, and MPS I-Hurler-Scheie Syndrome.
   79. The method according to paragraph 77, wherein the lysosomal storage disease is MPSII Hunter Syndrome.
   80. The method according to paragraph 73, wherein the infectious disease is selected from the group consisting of herpes simplex virus (HSV), such as HSV-1 and HSV-2, varicella zoster virus (VZV), Epstein-Barr virus (EBV), cytomegalovirus (CMV), human herpesvirus 6 (HHV-6), human herpesvirus 7 (HHV-7), hepatitis A virus (HAV), hepatitis B virus (HBV), hepatitis C virus (HCV), the delta hepatitis virus (HDV), hepatitis E virus (HEV), hepatitis G virus (HGV), Picornaviridae, Caliciviridae, Togaviridae, Flaviviridae, Coronaviridae, Reoviridae, Birnaviridae, Rhabodoviridae, Filoviridae, Paramyxoviridae, Orthomyxoviridae, Bunyaviridae, Arenaviridae, Retroviradae, lentiviruses, simian immunodeficiency virus (SIV), human papillomavirus (HPV), influenza virus and tick-borne encephalitis viruses.
   81. The method according to any one of paragraphs 55, 58, 61 and 64, wherein the vector is administered at a dose of about 1×10⁹ vg/kg to about 1×10¹⁷ vg/kg.
   82. The method according to paragraph 81, wherein the vector is administered at a dose selected from the group consisting of about 5×10¹² vg/kg, about 1×10¹³ vg/kg, about 5×10¹³ vg/kg and about 1×10¹⁴ vg/kg.
   83. The method according to any one of paragraphs 81-82, wherein the vector comprising the polynucleotide encoding one or more zinc finger nucleases is administered at a dose of about 1×10¹² vg/kg to about 1×10¹⁴ vg/kg.
   84. A method for correcting a disease-causing mutation in the genome of a cell, the method comprising modifying a target nucleotide sequence in the genome of the cell by introducing into the cell an effective amount of the polynucleotide construct according to any one of paragraphs 1-14.
   85. A method for correcting a disease-causing mutation in the genome of a cell, the method comprising modifying a target nucleotide sequence in the genome of the cell by introducing into the cell an effective amount of the vector according to any one of paragraphs 15-17.
   86. A method for correcting a disease-causing mutation in the genome of a cell, the method comprising modifying a target nucleotide sequence in the genome of the cell by introducing into the cell an effective amount of the pharmaceutical composition according to any one of paragraphs 31-53.
   87. The method according to paragraph 82, the method further comprising introducing into the cell an effective amount of a first polynucleotide encoding a first zinc finger nuclease (ZFN) and a second polynucleotide encoding a second zinc finger nuclease (ZFN).
   88. The method according to paragraph 83, the method further comprising introducing into the cell an effective amount of a first vector comprising a first polynucleotide encoding a first zinc finger nuclease (ZFN) and a second vector comprising a second polynucleotide encoding a second zinc finger nuclease (ZFN).
   89. The method according to paragraph 83, the method further comprising introducing into the cell an effective amount of a polynucleotide encoding one or more zinc finger nucleases (ZFN).
   90. The method according to paragraph 83, the method further comprising introducing into the cell an effective amount of a vector comprising a polynucleotide encoding one or more zinc finger nucleases (ZFN).
   91. The method according to any one of paragraphs 84-90, wherein upon integration of the polynucleotide construct of any one of paragraphs 1-14 into the genome of the cell, the first nucleotide sequence encoding the first polypeptide is expressed.
   92. The method according to any one of paragraphs 84-90, wherein upon integration of the polynucleotide construct of any one of paragraphs 1-14 into the genome of the cell, the second nucleotide sequence encoding the second polypeptide is expressed.
   93. The method according to any one of paragraphs 54-92, wherein the cell is a eukaryotic cell.
   94. The method according to paragraph 93, wherein the cell is a mammalian cell.
   95. The method according to paragraph 94, wherein the cell is a stem cell.
   96. The method according to paragraph 93, wherein the cell is a human cell.
   97. The method according to any one of paragraphs 54-96, wherein the cell is a non-dividing cell.
   98. The method according to paragraph 93, wherein the cell is a hepatocyte.
   99. The method according to any one of paragraphs 57-98, wherein the target nucleotide sequence is an endogenous locus.
   100. Use of a polynucleotide construct according to any one of paragraphs 1-14 for the preparation of a medicament for treating a disease or disorder.
   101. Use of a polynucleotide construct according to any one of paragraphs 1-14 for the preparation of a medicament for modifying the genome of a cell.
   102. Use of a polynucleotide construct according to any one of paragraphs 1-14 for the preparation of a medicament for integrating a transgene into a target nucleotide sequence of a cell.
   103. Use of a polynucleotide construct according to any one of paragraphs 1-14 for the preparation of a medicament for disrupting a target nucleotide sequence in a cell.
   104. Use of a polynucleotide construct according to any one of paragraphs 1-14 for the preparation of a medicament for correcting a disease-causing mutation in the genome of a cell.
   105. Use of a polynucleotide construct according to any one of paragraphs 1-14 for the preparation of a medicament for modifying a target nucleotide sequence in the genome of a cell.
   106. The polynucleotide construct of any one of paragraphs 1-14, for use in treating a disease or disorder.
   107. The polynucleotide construct of any one of paragraphs 1-14, for use in modifying the genome of a cell.
   108. The polynucleotide construct of any one of paragraphs 1-14, for use in integrating a transgene into a target nucleotide sequence of a cell.
   109. The polynucleotide construct of any one of paragraphs 1-14, for use in disrupting a target nucleotide sequence in a cell.
   110. The polynucleotide construct of any one of paragraphs 1-14, for use in correcting a disease-causing mutation in the genome of a cell.
   111. The polynucleotide construct of any one of paragraphs 1-14, for use in modifying a target nucleotide sequence in the genome of a cell.

### EXAMPLES

### Example 1: Assessment of Push-Pull IDS Constructs in iPS Derived Human Hepatocytes

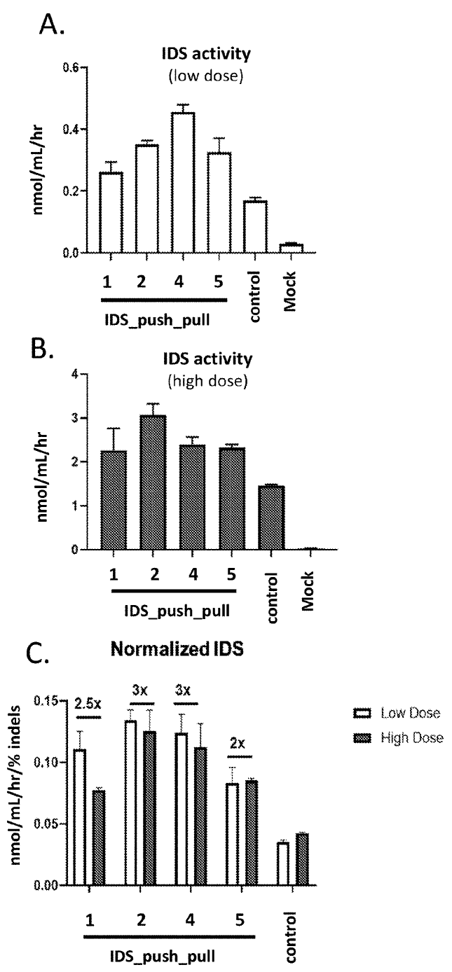

iPS derived human hepatocytes were transduced with zinc finger nuclease (ZFN) AAV constructs and various donor AAV constructs (1, 2, 4 and 5) comprising transgenes that encode for Iduronate-2-sulfatase (IDS) as indicated in FIG. 2. At least one or both of the transgenes were codon diversified. Sequences of the donor constructs are listed in Table 2. A low dose of 30 vg/cell of each ZFN AAV and 240 vg/cell of donor AAV (FIG. 3, Panel A), or a high dose of 300 vg/cells each ZFN AAV and 2400 vg/cell of donor AAV (FIG. 3, Panel B) were used to transduce the hepatocytes. Separate AAV vectors encoding left and right ZFNs were used. See, e.g., U.S. Patent Publication No. 2019/0241877. A single IDS donor was used as a control. See, e.g., U.S. patent application Ser. No. 16/534,280. IDS enzymatic activity was measured by the level of IDS produced by hepatocytes, as measured in nmol/mL/hour. IDS activity was normalized by percentage of insertions and deletions (% indels). The IDS activity of donor constructs IDS_push_pull 2 and IDS_push_pull 4 resulted in a 3-fold higher level of IDS production compared to the control. Donor construct, IDS_push_pull 1 had a 2.5-fold increase, while donor construct IDS_push_pull 5 had a 2-fold increase of IDS production compared to the control. See FIG. 3, Panel C.

What is claimed is:

1. A polynucleotide construct comprising in 5′ to 3′ orientation:
a. a first Inverted Terminal Repeat (ITR) nucleotide sequence;
b. a first nucleotide sequence encoding a first polypeptide;
c. a second nucleotide sequence encoding a second polypeptide; and
d. a second ITR nucleotide sequence;
wherein the first nucleotide sequence encoding a first polypeptide is oriented tail-to-tail to the second nucleotide sequence encoding a second polypeptide; and wherein the first nucleotide sequence and the second nucleotide sequence encode a polypeptide having the same amino acid sequence.

2. The polynucleotide construct according to claim 1, further comprising:
e. a first splice acceptor sequence operatively linked to the first nucleotide sequence encoding the first polypeptide; and
f. a second splice acceptor sequence operatively linked to the second nucleotide sequence encoding the second polypeptide.

3. The polynucleotide construct according to claim 2, wherein each of said first splice acceptor sequence and second splice acceptor sequence is independently selected from a Factor 9 Splice Acceptor (F9SA), a CFTR Splice acceptor, a COL5A2 Splice acceptor, a NF1 Splice Acceptor, a MLH1 Splice Acceptor, and an Albumin (ALB) Splice Acceptor.

4. The polynucleotide construct according to claim 1 or 2, further comprising:
g. a first polyadenylation (polyA) signal sequence operatively linked to the nucleotide sequence encoding the first polypeptide; and
h. a second polyadenylation (polyA) signal sequence operatively linked to the nucleotide sequence encoding the second polypeptide.

5. The polynucleotide construct according to claim 4, wherein the first polyA signal sequence is selected from a human Growth Hormone (hGH) polyA signal, a bovine Growth Hormone (bGH) polyA signal, a SV40 polyA signal, and a rbGlob polyA signal.

6. The polynucleotide construct according to claim 4 or 5, wherein the second polyA signal sequence is selected from a human Growth Hormone (hGH) polyA signal, a bovine Growth Hormone (bGH) polyA signal, a SV40 polyA signal, and a rbGlob polyA signal.

7. The polynucleotide construct according to any one of claims 1-6, wherein the nucleotide sequence encoding the first polypeptide or the nucleotide sequence encoding the second polypeptide encodes a therapeutic polypeptide.

8. The polynucleotide construct according to claim 7, wherein the therapeutic polypeptide is selected from the group consisting of iduronate-2-sulphatase (IDS), alpha-L-iduronidase (IDUA), alpha-D-mannosidase, N-aspartyl-beta-glucosaminidase, lysosomal acid lipase, cystinosin, lysosomal associated membrane protein 2, alpha-galactosidase A, acid ceramidase, alpha fucosidase, cathepsin A, acid beta-glucocerebrosidase, beta galactosidase, beta hexosaminidase A, beta hexosaminidase B, beta hexosaminidase, GM2 ganglioside activator, GLcNAc-1-phosphotransferase, Beta-galactosylceramidase, arylsulfatase A, heparan N-sulfatase, alpha-N-acetylglucosaminidase, acetyl CoA:alpha-glucosaminide acetyltransferase, N-acetyl glucosamine-6-sulfatase, arylsulfatase B, beta-glucuronidase, hyaluronidase, neuraminidase, mucolipin-1, formylglycine-generating enzyme, palmitoyl-protein thioesterase 1, tripeptidyl peptidase 1, CLN3 protein, cysteine string protein alpha, CLN5 protein, CLN6 protein, CLN7 protein, CLN8 protein, acid sphingomyelinase, NPC 1, NPC 2, phenylalanine hydroxylase, acid alpha-glucosidase, cathepsin K, sialin, alpha-N-acetylgalactosaminidase, glucose-6-phosphatase, solute carrier family 37 member 4, argininosuccinate synthase 1, solute carrier family 25 member 13, and ornithine transcarbamylase.

9. The polynucleotide construct according to any one of claims 1-8, wherein the nucleotide sequence encoding the first polypeptide is codon diversified.

10. The polynucleotide construct according to any one of claims 1-9, wherein the nucleotide sequence encoding the second polypeptide is codon diversified.

11. The polynucleotide construct according to any one of claims 1-10, wherein each of the nucleotide sequence encoding the first polypeptide and the nucleotide sequence encoding the second polypeptide is each independently codon diversified.

12. The polynucleotide construct according to any one of claims 1-11, wherein the nucleotide sequence encoding the first polypeptide comprises the nucleotide sequence set forth in any one of SEQ ID NOs: 184-193.

13. The polynucleotide construct according to any one of claims 1-12, wherein the nucleotide sequence encoding the second polypeptide comprises the nucleotide sequence set forth in any one of SEQ ID NOs: 184-193.

14. The polynucleotide construct according to claim 1, wherein said polynucleotide construct comprises the nucleotide sequence set forth in any one of SEQ ID NOs: 173-176.

15. A vector comprising the polynucleotide construct according to any one of claims 1-14.

16. The vector according to claim 15, wherein the vector is an adeno-associated viral (AAV) vector.

17. The vector according to claim 16, wherein the AAV is selected from the group consisting of AAV-MeCP2, AAV1, AAV2, AAV3, AAV4, AAV5, AAV6, AAV8, AAV8.2, AAV9, Dual AAV9, AAVrh8, AAVrh10, AAHrh43, AAVhu37, AAV2/8, AAV2/5, and AAV2/6.

18. A cell comprising the polynucleotide construct according to any one of claims 1-14 or the vector according to any one of claims 15-17.

19. The cell according to claim 18, wherein the cell is a eukaryotic cell.

20. The cell according to claim 19, wherein the cell is a mammalian cell.

21. The cell according to claim 20, wherein the cell is a stem cell.

22. The cell according to claim 19, wherein the cell is a human cell.

23. The cell according to any one of claims 18-22, wherein the cell is a non-dividing cell.

24. The cell according to any one of claims 19-23, wherein the cell is a hepatocyte.

25. The cell according to any one of claims 18-24, wherein the cell further comprises a polynucleotide encoding a nuclease.

26. The cell according to any one of claims 18-24, wherein the cell further comprises a first polynucleotide encoding a first zinc finger nuclease (ZFN) and a second polynucleotide encoding a second zinc finger nuclease (ZFN).

27. The cell according to any one of claims 18-24, wherein the cell further comprises a first vector comprising a first polynucleotide encoding a first zinc finger nuclease (ZFN) and a second vector comprising a second polynucleotide encoding a second zinc finger nuclease (ZFN).

28. The cell according to any one of claims 18-24, wherein the cell further comprises a polynucleotide encoding one or more zinc finger nucleases (ZFN).

29. The cell according to any one of claims 18-24, wherein the cell further comprises a vector comprising a polynucleotide encoding one or more zinc finger nucleases (ZFN).

30. The cell according to claim 28 or 29, wherein the zinc finger nuclease is a 2-in-1 zinc finger nuclease.

31. A pharmaceutical composition comprising the polynucleotide construct according to any one of claims 1-14; and a pharmaceutically acceptable carrier.

32. The pharmaceutical composition according to claim 31, wherein the composition further comprises a first polynucleotide encoding a first zinc finger nuclease (ZFN) and a second polynucleotide encoding a second zinc finger nuclease (ZFN).

33. The pharmaceutical composition according to claim 31, wherein the composition comprises a first vector comprising a first polynucleotide encoding a first zinc finger nuclease (ZFN) and a second vector comprising a second polynucleotide encoding a second zinc finger nuclease (ZFN).

34. The pharmaceutical composition according to claim 31, wherein the composition further comprises a polynucleotide encoding one or more zinc finger nucleases (ZFN).

35. The pharmaceutical composition according to claim 32, wherein the composition further comprises a vector comprising a polynucleotide encoding one or more zinc finger nucleases (ZFN).

36. The pharmaceutical composition according to claims 32, 34-35, wherein the zinc finger nuclease is a 2-in-1 zinc finger nuclease.

37. The pharmaceutical composition according to claim 32, wherein the ratio of the polynucleotide encoding the first zinc finger nuclease: the polynucleotide encoding the second zinc finger: the polynucleotide of any one of claims 1-14 is 1:1:8.

38. The pharmaceutical composition according to claim 32, wherein the ratio of the polynucleotide encoding the first zinc finger nuclease: the polynucleotide encoding the second zinc finger: the polynucleotide of any one of claims 1-14 is 1:1:4.

39. The pharmaceutical composition according to claim 32, wherein the ratio of the polynucleotide encoding the first zinc finger nuclease: the polynucleotide encoding the second zinc finger: the polynucleotide of any one of claims 1-14 is 1:1:2.

40. The pharmaceutical composition according to claim 32, wherein the ratio of the polynucleotide encoding the first zinc finger nuclease: the polynucleotide encoding the second zinc finger: the polynucleotide of any one of claims 1-14 is 3:3:4.

41. The pharmaceutical composition according to claim 33, wherein the ratio of the vector comprising the first polynucleotide encoding the first zinc finger nuclease: the vector comprising the polynucleotide encoding the second zinc finger: the vector of any one of claims 15-17 is 1:1:8.

42. The pharmaceutical composition according to claim 33, wherein the ratio of the vector comprising the first polynucleotide encoding the first zinc finger nuclease: the vector comprising the polynucleotide encoding the second zinc finger: the vector of any one of claims 15-17 is 1:1:4.

43. The pharmaceutical composition according to claim 33, wherein the ratio of the vector comprising the first polynucleotide encoding the first zinc finger nuclease: the vector comprising the polynucleotide encoding the second zinc finger: the vector of any one of claims 15-17 is 1:1:2.

44. The pharmaceutical composition according to claim 33, wherein the ratio of the vector comprising the first polynucleotide encoding the first zinc finger nuclease: the vector comprising the polynucleotide encoding the second zinc finger: the vector of any one of claims 15-17 is 3:3:4.

45. The pharmaceutical composition according to claim 36, wherein the ratio of the polynucleotide encoding the 2-in-1 zinc finger nuclease: the polynucleotide construct of any one of claims 1-14 is 1:4.

46. The pharmaceutical composition according to claim 36, wherein the ratio of the polynucleotide encoding the 2-in-1 zinc finger nuclease: the polynucleotide construct of any one of claims 1-14 is 1:2.

47. The pharmaceutical composition according to claim 36, wherein the ratio of the polynucleotide encoding the 2-in-1 zinc finger nuclease: the polynucleotide construct of any one of claims 1-14 is 1:1.

48. The pharmaceutical composition according to claim 36, wherein the ratio of the polynucleotide encoding the 2-in-1 zinc finger nuclease: the polynucleotide construct of any one of claims 1-14 is 3:2.

49. The pharmaceutical composition according to claim 33, wherein the ratio of the vector comprising the 2-in-1 zinc finger nuclease: the vector of any one of claims 15-17 is 1:4.

50. The pharmaceutical composition according to claim 33, wherein the ratio of the vector comprising the 2-in-1 zinc finger nuclease: the vector of any one of claims 15-17 is 1:2.

51. The pharmaceutical composition according to claim 33, wherein the ratio of the vector comprising the 2-in-1 zinc finger nuclease: the vector of any one of claims 15-17 is 1:1.

52. The pharmaceutical composition according to claim 33, wherein the ratio of the vector comprising the 2-in-1 zinc finger nuclease: the vector of any one of claims 15-17 is 3:2.

53. The pharmaceutical composition of any one of claims 31-52, wherein the composition is formulated for intravenous, intramuscular, subcutaneous, or intrathecal administration.

54. A method for modifying the genome of a cell, the method comprising introducing into a cell an effective amount of the polynucleotide construct according to any one of claims 1-14.

55. A method for modifying the genome of a cell, the method comprising introducing into a cell an effective amount of the vector according to any one of claims 15-17.

56. A method for modifying the genome of a cell, the method comprising introducing into a cell an effective amount of the pharmaceutical composition according to any one of claims 31-53.

57. A method for integrating an exogenous nucleotide sequence into a target nucleotide sequence of a cell, the method comprising introducing into a cell an effective amount of the polynucleotide construct according to any one of claims 1-14.

58. A method for integrating an exogenous nucleotide sequence into a target nucleotide sequence of a cell, the method comprising introducing into a cell an effective amount of the vector according to any one of claims 15-17.

59. A method for integrating an exogenous nucleotide sequence into a target nucleotide sequence of a cell, the method comprising introducing into a cell an effective amount of the pharmaceutical composition according to any one of claims 31-53.

60. A method for disrupting a target nucleotide sequence in a cell, the method comprising introducing into a cell an effective amount of the polynucleotide construct according to any one of claims 1-14.

61. A method for disrupting a target nucleotide sequence in a cell, the method comprising introducing into a cell an effective amount of the vector according to any one of claims 15-17.

62. A method for disrupting a target nucleotide sequence in a cell, the method comprising introducing into a cell an effective amount of the pharmaceutical composition according to any one of claims 31-53.

63. A method for treating a disorder in a subject, the method comprising modifying a target nucleotide sequence in the genome of a cell of said subject by introducing into the cell an effective amount of the polynucleotide construct according to any one of claims 1-14.

64. A method for treating a disorder in a subject, the method comprising modifying a target nucleotide sequence in the genome of a cell of said subject by introducing into the cell an effective amount of the vector according to any one of claims 15-17.

65. A method for treating a disorder in a subject, the method comprising modifying a target nucleotide sequence in the genome of a cell of said subject by introducing into the cell an effective amount of the pharmaceutical composition according to any one of claims 31-53.

66. The method according to any one of claims 54, 57, 60 and 63, the method further comprising introducing into the cell an effective amount of a first polynucleotide encoding a first zinc finger nuclease (ZFN) and a second polynucleotide encoding a second zinc finger nuclease (ZFN).

67. The method according to any one of claims 55, 58, 61 and 64, the method further comprising introducing into the cell an effective amount of a first vector comprising a first polynucleotide encoding a first zinc finger nuclease (ZFN) and a second vector comprising a second polynucleotide encoding a second zinc finger nuclease (ZFN).

68. The method according to any one of claims 54, 57, 60 and 63, the method further comprising introducing into the cell an effective amount of a polynucleotide encoding one or more zinc finger nucleases (ZFN).

69. The method according to any one of claims 55, 58, 61 and 64, the method further comprising introducing into the cell an effective amount of a vector comprising a polynucleotide encoding one or more zinc finger nucleases (ZFN).

70. The method according to claim 68-69, wherein the zinc finger nuclease is a 2-in-1 zinc finger nuclease.

71. The method according to any one of claims 54-70, wherein upon integration of the polynucleotide construct of any one of claims 1-14 into the genome of the cell, the first nucleotide sequence encoding the first polypeptide is expressed.

72. The method according to any one of claims 54-70, wherein upon integration of the polynucleotide construct of any one of claims 1-14 into the genome of the cell, the second nucleotide sequence encoding the second polypeptide is expressed.

73. The method according to any one of claim 63-72, wherein the disorder is selected from the group consisting of a, a genetic disorder, an infectious disease, an acquired disorder, and a cancer.

74. The method according to claim 73, wherein the genetic disorder is selected from the group consisting of achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangiectasia, barth syndrome, beta-thalassemia, blue rubber bleb nevus syndrome, canavan disease, chronic granulomatous diseases (CGD), citrullinemia, cri du chat syndrome, cystic fibrosis, dercum's disease, ectodermal dysplasia, Fabry disease, fanconi anemia, fibrodysplasia ossificans progressive, fragile X syndrome, galactosemis, Gaucher's disease, generalized gangliosidoses (e.g., GM1), GSD (e.g., GSD1a) hemochromatosis, the hemoglobin C mutation in the 6th codon of beta-globin (HbC), hemophilia, Hunter syndrome, Huntington's disease, Hurler Syndrome, hypophosphatasia, Klinefelter syndrome, Krabbes Disease, Langer-Giedion Syndrome, leukocyte adhesion deficiency (LAD, OMIM No. 116920), leukodystrophy, long QT syndrome, lipoprotein lipase deficiency, Marfan syndrome, Moebius syndrome, mucopolysaccharidosis (MPS), nail patella syndrome, nephrogenic diabetes insipdius, neurofibromatosis, Neimann-Pick disease, ornithine transcarbamylase (OTC) deficiency, osteogenesis imperfecta, phenylketonuria (PKU), Pompe disease, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, and X-linked lymphoproliferative syndrome (XLP, OMIM No. 308240).

75. The method according to claim 73, wherein the genetic disorder is a lysosomal storage disease.

76. The method according to claim 75, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/J/A), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.

77. The method according to claim 76, wherein the lysosomal storage disease is selected from MPSI and MPSII.

78. The method according to claim 77, wherein the lysosomal storage disease is selected from the group consisting of MPS I—Hurler Syndrome, MPS I—Scheie Syndrome, and MPS I-Hurler-Scheie Syndrome.

79. The method according to claim 77, wherein the lysosomal storage disease is MPSII Hunter Syndrome.

80. The method according to claim 73, wherein the infectious disease is selected from the group consisting of herpes simplex virus (HSV), such as HSV-1 and HSV-2, varicella zoster virus (VZV), Epstein-Barr virus (EBV), cytomegalovirus (CMV), human herpesvirus 6 (HHV-6), human herpesvirus 7 (HHV-7), hepatitis A virus (HAV), hepatitis B virus (HBV), hepatitis C virus (HCV), the delta hepatitis virus (HDV), hepatitis E virus (HEV), hepatitis G virus (HGV), Picornaviridae, Caliciviridae, Togaviridae, Flaviviridae, Coronaviridae, Reoviridae, Birnaviridae, Rhabodoviridae, Filoviridae, Paramyxoviridae, Orthomyxoviridae, Bunyaviridae, Arenaviridae, Retroviradae, lentiviruses, simian immunodeficiency virus (SIV), human papillomavirus (HPV), influenza virus and tick-borne encephalitis viruses.

81. The method according to any one of claims 55, 58, 61 and 64, wherein the vector is administered at a dose of about 1×10⁹ vg/kg to about 1×10¹⁷ vg/kg.

82. The method according to claim 81, wherein the vector is administered at a dose selected from the group consisting of about 5×10¹² vg/kg, about 1×10¹³ vg/kg, about 5×10¹³ vg/kg and about 1×10¹⁴ vg/kg.

83. The method according to any one of claims 81-82, wherein the vector comprising the polynucleotide encoding one or more zinc finger nucleases is administered at a dose of about 1×10¹² vg/kg to about 1×10¹⁴ vg/kg.

84. A method for correcting a disease-causing mutation in the genome of a cell, the method comprising modifying a target nucleotide sequence in the genome of the cell by introducing into the cell an effective amount of the polynucleotide construct according to any one of claims 1-14.

85. A method for correcting a disease-causing mutation in the genome of a cell, the method comprising modifying a target nucleotide sequence in the genome of the cell by introducing into the cell an effective amount of the vector according to any one of claims 15-17.

86. A method for correcting a disease-causing mutation in the genome of a cell, the method comprising modifying a target nucleotide sequence in the genome of the cell by introducing into the cell an effective amount of the pharmaceutical composition according to any one of claims 31-53.

87. The method according to claim 82, the method further comprising introducing into the cell an effective amount of a first polynucleotide encoding a first zinc finger nuclease (ZFN) and a second polynucleotide encoding a second zinc finger nuclease (ZFN).

88. The method according to claim 83, the method further comprising introducing into the cell an effective amount of a first vector comprising a first polynucleotide encoding a first zinc finger nuclease (ZFN) and a second vector comprising a second polynucleotide encoding a second zinc finger nuclease (ZFN).

89. The method according to claim 83, the method further comprising introducing into the cell an effective amount of a polynucleotide encoding one or more zinc finger nucleases (ZFN).

90. The method according to claim 83, the method further comprising introducing into the cell an effective amount of a vector comprising a polynucleotide encoding one or more zinc finger nucleases (ZFN).

91. The method according to any one of claims 84-90, wherein upon integration of the polynucleotide construct of any one of claims 1-14 into the genome of the cell, the first nucleotide sequence encoding the first polypeptide is expressed.

92. The method according to any one of claims 84-90, wherein upon integration of the polynucleotide construct of any one of claims 1-14 into the genome of the cell, the second nucleotide sequence encoding the second polypeptide is expressed.

93. The method according to any one of claims 54-92, wherein the cell is a eukaryotic cell.

94. The method according to claim 93, wherein the cell is a mammalian cell.

95. The method according to claim 94, wherein the cell is a stem cell.

96. The method according to claim 93, wherein the cell is a human cell.

97. The method according to any one of claims 54-96, wherein the cell is a non-dividing cell.

98. The method according to claim 93, wherein the cell is a hepatocyte.

99. The method according to any one of claims 57-98, wherein the target nucleotide sequence is an endogenous locus.

100. Use of a polynucleotide construct according to any one of claims 1-14 for the preparation of a medicament for treating a disease or disorder.

101. Use of a polynucleotide construct according to any one of claims 1-14 for the preparation of a medicament for modifying the genome of a cell.

102. Use of a polynucleotide construct according to any one of claims 1-14 for the preparation of a medicament for integrating a transgene into a target nucleotide sequence of a cell.

103. Use of a polynucleotide construct according to any one of claims 1-14 for the preparation of a medicament for disrupting a target nucleotide sequence in a cell.

104. Use of a polynucleotide construct according to any one of claims 1-14 for the preparation of a medicament for correcting a disease-causing mutation in the genome of a cell.

105. Use of a polynucleotide construct according to any one of claims 1-14 for the preparation of a medicament for modifying a target nucleotide sequence in the genome of a cell.

106. The polynucleotide construct of any one of claims 1-14, for use in treating a disease or disorder.

107. The polynucleotide construct of any one of claims 1-14, for use in modifying the genome of a cell.

108. The polynucleotide construct of any one of claims 1-14, for use in integrating a transgene into a target nucleotide sequence of a cell.

109. The polynucleotide construct of any one of claims 1-14, for use in disrupting a target nucleotide sequence in a cell.

110. The polynucleotide construct of any one of claims 1-14, for use in correcting a disease-causing mutation in the genome of a cell.

111. The polynucleotide construct of any one of claims 1-14, for use in modifying a target nucleotide sequence in the genome of a cell.


SEQUENCE LISTING (193 sequences)

SEQ ID NO: 1 - protein, 7 aa, Artificial Sequence
  source: /note=“Description of Artificial Sequence Synthetic peptide”
DYKDDDK

SEQ ID NO: 2 - protein, 22 aa, Artificial Sequence
  source: /note=“Description of Artificial Sequence Synthetic peptide”
DYKDHDGDYKDHDIDYKDDDDK

SEQ ID NO: 3 - protein, 7 aa, Artificial Sequence
  source: /note=“Description of Artificial Sequence Synthetic peptide”
PKKKRKV

SEQ ID NO: 4 - protein, 9 aa, Artificial Sequence
  source: /note=“Description of Artificial Sequence Synthetic peptide”
PAAKRVKLD

SEQ ID NO: 5 - protein, 10 aa, Artificial Sequence
  source: /note=“Description of Artificial Sequence Synthetic peptide”
EGAPPAKRAR

SEQ ID NO: 6 - protein, 8 aa, Artificial Sequence
  source: /note=“Description of Artificial Sequence Synthetic peptide”
VSRKRPRP

SEQ ID NO: 7 - protein, 18 aa, Artificial Sequence
  source: /note=“Description of Artificial Sequence Synthetic peptide”
KRPAATKKAGQAKKKKLD

SEQ ID NO: 8 - protein, 38 aa, Artificial Sequence
  source: /note=“Description of Artificial Sequence Synthetic polypeptide”
NQSSNFGPMKGGNFGGRSSGPYGGGGQYFAKPRNQGGY

SEQ ID NO: 9 - protein, 17 aa, Artificial Sequence
  source: /note=“Description of Artificial Sequence Synthetic peptide”
PKTRRRPRRSQRKRPPT

SEQ ID NO: 10 - DNA, 130 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 11 - DNA, 321 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 12 - DNA, 393 bp, Artificial Sequence
  source: /note=“Description of Artificial Sequence Synthetic polynucleotide”
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 13 - DNA, 48 bp, Artificial Sequence
  source: /note=“Description of Artificial Sequence Synthetic oligonucleotide”
cttgttctttttgcagaagctcagaataaacgctcaactttggcagat

SEQ ID NO: 14 - DNA, 133 bp, Artificial Sequence
  source: /note=“Description of Artificial Sequence Synthetic polynucleotide”
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 15 - DNA, 66 bp, Artificial Sequence
  source: /note=“Description of Artificial Sequence Synthetic oligonucleotide”
gactacaaagaccatgacggtgattataaagatcatgacatcgattacaaggatgacgatgacaag

SEQ ID NO: 16 - DNA, 66 bp, Artificial Sequence
  source: /note=“Description of Artificial Sequence Synthetic oligonucleotide”
gattataaagatcatgacggggactataaggatcacgacatagactacaaagacgatgatgacaaa

SEQ ID NO: 17 - DNA, 1143 bp, Artificial Sequence
  source: /note=“Description of Artificial Sequence Synthetic polynucleotide”
gactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 18 - DNA, 1143 bp, Artificial Sequence
  source: /note=“Description of Artificial Sequence Synthetic polynucleotide”
gattacaaagatcacgacggagattacaaagatcacgacattgactataaggacgacgacgataaaatggctccaaagaagaaaagaaaagtggggatccatggtgtacccgcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgcggcagccagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtct

SEQ ID NO: 19 - DNA, 1143 bp, Artificial Sequence
  source: /note=“Description of Artificial Sequence Synthetic polynucleotide”
gactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagt

SEQ ID NO: 20 - DNA, 1143 bp, Artificial Sequence
  source: /note=“Description of Artificial Sequence Synthetic polynucleotide”
gattacaaagaccacgacggagactacaaggaccatgatattgactacaaagacgatgatgataagatggcacccaaaaagaagagaaaagtgggaatccacggtgtaccggccgcgatggcagagagaccatttcagtgtagaatctgtatgcagaacttttcccaatcaggaaacctggcacgacacattagaacccatactggagaaaagccgttcgcttgcgacatttgcggtagaaaatttgctttgaaacagaacttgtgtatgcataccaagattcataccggcgaaaaaccatttcaatgcaggatttgtatgcagaagttcgcctggcaatccaatttgcagaatcatactaaaattcataccggagaaaaaccattccaatgccgcatttgtatgagaaacttttctacctctggcaatctcaccagacatatcagaacacacacaggcgagaaaccgttcgcatgcgatatctgtgggcgaaagtttgccagaagatcccatctcacatcacatactaaaatacatttgcgaggaagtcaactggtcaagtccgaactggaggaaaaaaaaagtgagctgcgacacaagttgaagtacgtaccacacgaatacatcgagctgattgagatagcacggaactctacccaggatagaatactggagatgaaagttatggaattctttatgaaggtgtacggatacagggggaagcatcttggcgggagccggaaaccagacggagcaatctataccgtcgggtcacctatagactatggagttattgtcgatacaaaggcctattcaggaggttataatctgccaatcggccaagccgacgagatggagaggtacgtggaggaaaatcagaccagagacaagcacctgaaccctaatgaatggtggaaagtgtaccctagcagcgtcactgagttcaaattcctgttcgtcagcggtcattttaaaggaaattataaagcccagctcactagactcaaccatattacaaactgcgacggagccgtacttagcgttgaagagttgcttatcggaggagagatgatcaaagccggaaccctcacacttgaagaagtgcgaagaaaattcaataacggagagataaattttaggagt

SEQ ID NO: 21 - DNA, 1143 bp, Artificial Sequence
  source: /note=“Description of Artificial Sequence Synthetic polynucleotide”
gactataaagaccacgatggcgactacaaagaccacgacatcgattacaaggacgatgatgacaaaatggcacctaagaagaagagaaaagttggaatacatggagtccccgcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcgaggctcacagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagt

SEQ ID NO: 22 - DNA, 1143 bp, Artificial Sequence
  source: /note=“Description of Artificial Sequence Synthetic polynucleotide”
gattataaggaccatgacggagactataaagaccatgatattgactacaaagacgacgatgataagatggcccccaagaagaaacgaaaagtaggaatccatggcgtgcctgcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgcggtagccaactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagt

SEQ ID NO: 23 - DNA, 1143 bp, Artificial Sequence
  source: /note=“Description of Artificial Sequence Synthetic polynucleotide”
gactataaggaccatgatggagactataaagatcacgatattgactataaagatgatgatgataagatggcacctaagaagaaaagaaaggtcggcattcatggtgtgcctgcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcgaggaagccagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctcc

SEQ ID NO: 24 - DNA, 63 bp, Artificial Sequence
  source: /note=“Description of Artificial Sequence Synthetic oligonucleotide”
ggcagcggagagggcagaggaagcctgctcacctgcggtgacgtggaggaaaaccctggccct

SEQ ID NO: 25 - DNA, 1221 bp, Artificial Sequence
  source: /note=“Description of Artificial Sequence Synthetic polynucleotide”
gactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 26 - DNA, 1221 bp, Artificial Sequence
  source: /note=“Description of Artificial Sequence Synthetic polynucleotide”
gattataaagatcatgacggggactataaggatcacgacatagactacaaagacgatgatgacaaaatggcgcctaaaaagaaacgaaaagtgggcattcacggcgtacctgctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcagaggaagtcagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaacttt

SEQ ID NO: 27 - DNA, 1221 bp, Artificial Sequence
  source: /note=“Description of Artificial Sequence Synthetic polynucleotide”
gattataaagaccatgatggtgattacaaggaccatgacatcgattataaagacgacgacgacaaaatggcccctaagaaaaagagaaaagtcggaatccacggtgtcccagctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcggggttctcaactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaatttt

SEQ ID NO: 28 - DNA, 1221 bp, Artificial Sequence
  source: /note=“Description of Artificial Sequence Synthetic polynucleotide”
gattataaggatcatgatggagactataaggatcatgacatagattacaaagatgacgatgacaagatggcacccaagaagaaaagaaaagtaggaattcacggagtccctgccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgagaggcagtcagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaatttt

SEQ ID NO: 29 - DNA, 1221 bp, Artificial Sequence
  source: /note=“Description of Artificial Sequence Synthetic polynucleotide”
gactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttt

SEQ ID NO: 30 - DNA, 1221 bp, Artificial Sequence
  source: /note=“Description of Artificial Sequence Synthetic polynucleotide”
gattacaaagaccatgatggcgactataaagaccatgacatcgactacaaggatgatgatgataaaatggctccaaagaaaaagaggaaggtgggaatacatggagtaccagcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcggggttcacaactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttc

SEQ ID NO: 31 - DNA, 1221 bp, Artificial Sequence
  source: /note=“Description of Artificial Sequence Synthetic polynucleotide”
gactacaaggaccacgacggagactataaagaccatgatatagattacaaggacgatgacgataaaatggcacccaaaaagaaaagaaaggtgggtattcacggagttcccgctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgagaggttcccagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttc

SEQ ID NO: 32 - DNA, 592 bp, Artificial Sequence
  source: /note=“Description of Artificial Sequence Synthetic polynucleotide”
aatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctg

SEQ ID NO: 33 - DNA, 223 bp, Artificial Sequence
  source: /note=“Description of Artificial Sequence Synthetic polynucleotide”
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctat

SEQ ID NO: 34 - DNA, 108 bp, Artificial Sequence
  source: /note=“Description of Artificial Sequence Synthetic polynucleotide”
aggaacccctagtgatggagttggccactccctctctgcgcgctcgctcgctcactgaggccgcccgggctttgcccgggcggcctcagtgagcgagcgagcgcgcag

SEQ ID NO: 35 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggattataaagatcatgacggggactataaggatcacgacatagactacaaagacgatgatgacaaaatggcgcctaaaaagaaacgaaaagtgggcattcacggcgtacctgctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcagaggaagtcagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaactttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 36 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggattataaagaccatgatggtgattacaaggaccatgacatcgattataaagacgacgacgacaaaatggcccctaagaaaaagagaaaagtcggaatccacggtgtcccagctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcggggttctcaactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaattttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 37 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggattataaggatcatgatggagactataaggatcatgacatagattacaaagatgacgatgacaagatggcacccaagaagaaaagaaaagtaggaattcacggagtccctgccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgagaggcagtcagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaattttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 38 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaactttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 39 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggattacaaagaccatgatggcgactataaagaccatgacatcgactacaaggatgatgatgataaaatggctccaaagaaaaagaggaaggtgggaatacatggagtaccagcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcggggttcacaactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttcggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 40 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaggaccacgacggagactataaagaccatgatatagattacaaggacgatgacgataaaatggcacccaaaaagaaaagaaaggtgggtattcacggagttcccgctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgagaggttcccagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttcggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 41 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttcggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 42 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggattacaaagatcacgacggagattacaaagatcacgacattgactataaggacgacgacgataaaatggctccaaagaagaaaagaaaagtggggatccatggtgtacccgcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgcggcagccagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtctggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 43 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactataaagaccacgatggcgactacaaagaccacgacatcgattacaaggacgatgatgacaaaatggcacctaagaagaagagaaaagttggaatacatggagtccccgcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcgaggctcacagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagtggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 44 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggattataaggaccatgacggagactataaagaccatgatattgactacaaagacgacgatgataagatggcccccaagaagaaacgaaaagtaggaatccatggcgtgcctgcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgcggtagccaactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagtggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 45 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactataaggaccatgatggagactataaagatcacgatattgactataaagatgatgatgataagatggcacctaagaagaaaagaaaggtcggcattcatggtgtgcctgcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcgaggaagccagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctccggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 46 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatctggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 47 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagtggcagcggagagggcagaggaagcctgctcacctgcggtgacgtggaggaaaaccctggccctacgcgtgccatggactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 48 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaactttggcagcggagagggcagaggaagcctgctcacctgcggtgacgtggaggaaaaccctggccctacgcgtgccatggactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 49 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagtggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 50 - DNA, 66 bp, Artificial Sequence
  source: /note=“Description of Artificial Sequence Synthetic oligonucleotide”
gattataaagaccatgatggtgattacaaggaccatgacatcgattataaagacgacgacgacaaa

SEQ ID NO: 51 - DNA, 66 bp, Artificial Sequence
  source: /note=“Description of Artificial Sequence Synthetic oligonucleotide”
gattataaggatcatgatggagactataaggatcatgacatagattacaaagatgacgatgacaag

SEQ ID NO: 52 - DNA, 66 bp, Artificial Sequence
  source: /note=“Description of Artificial Sequence Synthetic oligonucleotide”
gactacaaagatcatgatggcgactacaaagatcatgatatagattacaaagacgatgacgacaaa

SEQ ID NO: 53 - DNA, 66 bp, Artificial Sequence
  source: /note=“Description of Artificial Sequence Synthetic oligonucleotide”
gattacaaagaccatgatggcgactataaagaccatgacatcgactacaaggatgatgatgataaa

SEQ ID NO: 54 - DNA, 66 bp, Artificial Sequence
  source: /note=“Description of Artificial Sequence Synthetic oligonucleotide”
gactacaaggaccacgacggagactataaagaccatgatatagattacaaggacgatgacgataaa

SEQ ID NO: 55 - DNA, 66 bp, Artificial Sequence
  source: /note=“Description of Artificial Sequence Synthetic oligonucleotide”
gactacaaggaccacgacggtgactacaaagaccacgatatagactataaagatgacgatgataag

SEQ ID NO: 56 - DNA, 66 bp, Artificial Sequence
  source: /note=“Description of Artificial Sequence Synthetic oligonucleotide”
gactataaagaccacgatggcgactacaaagaccacgacatcgattacaaggacgatgatgacaaa

SEQ ID NO: 57 - DNA, 66 bp, Artificial Sequence
  source: /note=“Description of Artificial Sequence Synthetic oligonucleotide”
gattataaggaccatgacggagactataaagaccatgatattgactacaaagacgacgatgataag

SEQ ID NO: 58 - DNA, 66 bp, Artificial Sequence
  source: /note=“Description of Artificial Sequence Synthetic oligonucleotide”
gactataaggaccatgatggagactataaagatcacgatattgactataaagatgatgatgataag

SEQ ID NO: 59 - DNA, 30 bp, Artificial Sequence
  source: /note=“Description of Artificial Sequence Synthetic oligonucleotide”
cctaaaaagaaacgaaaagtgggcattcac

SEQ ID NO: 60 - DNA, 30 bp, Artificial Sequence
  source: /note=“Description of Artificial Sequence Synthetic oligonucleotide”
cccaagaagaagaggaaggtcggcattcat

SEQ ID NO: 61 - DNA, 30 bp, Artificial Sequence
  source: /note=“Description of Artificial Sequence Synthetic oligonucleotide”
cctaagaaaaagagaaaagtcggaatccac

SEQ ID NO: 62 - DNA, 30 bp, Artificial Sequence
  source: /note=“Description of Artificial Sequence Synthetic oligonucleotide”
cccaagaagaaaagaaaagtaggaattcac

SEQ ID NO: 63 - DNA, 30 bp, Artificial Sequence
  source: /note=“Description of Artificial Sequence Synthetic oligonucleotide”
ccaaaaaaaaaacgcaaggttggaatacac

SEQ ID NO: 64 - DNA, 30 bp, Artificial Sequence
  source: /note=“Description of Artificial Sequence Synthetic oligonucleotide”
ccaaagaaaaagaggaaggtgggaatacat

SEQ ID NO: 65 - DNA, 30 bp, Artificial Sequence
  source: /note=“Description of Artificial Sequence Synthetic oligonucleotide”
cccaaaaagaaaagaaaggtgggtattcac

SEQ ID NO: 66 - DNA, 30 bp, Artificial Sequence
  source: /note=“Description of Artificial Sequence Synthetic oligonucleotide”
ccaaagaagaaaagaaaagtggggatccat

SEQ ID NO: 67 - DNA, 30 bp, Artificial Sequence
  source: /note=“Description of Artificial Sequence Synthetic oligonucleotide”
cctaaaaaaaagcggaaagtgggaattcac

SEQ ID NO: 68 - DNA, 30 bp, Artificial Sequence
  source: /note=“Description of Artificial Sequence Synthetic oligonucleotide”
cctaagaagaagagaaaagttggaatacat

SEQ ID NO: 69 - DNA, 30 bp, Artificial Sequence
  source: /note=“Description of Artificial Sequence Synthetic oligonucleotide”
cccaagaagaaacgaaaagtaggaatccat

SEQ ID NO: 70 - DNA, 30 bp, Artificial Sequence
  source: /note=“Description of Artificial Sequence Synthetic oligonucleotide”
cctaagaagaaaagaaaggtcggcattcat

SEQ ID NO: 71 - DNA, 1032 bp, Artificial Sequence
  source: /note=“Description of Artificial Sequence Synthetic polynucleotide”
gccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 72 - DNA, 1032 bp, Artificial Sequence
  source: /note=“Description of Artificial Sequence Synthetic polynucleotide”
gcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgcggcagccagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtct

SEQ ID NO: 73 - DNA, 1032 bp, Artificial Sequence
  source: /note=“Description of Artificial Sequence Synthetic polynucleotide”
gccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagt

SEQ ID NO: 74 - DNA, 1032 bp, Artificial Sequence
  source: /note=“Description of Artificial Sequence Synthetic polynucleotide”
gccgcgatggcagagagaccatttcagtgtagaatctgtatgcagaacttttcccaatcaggaaacctggcacgacacattagaacccatactggagaaaagccgttcgcttgcgacatttgcggtagaaaatttgctttgaaacagaacttgtgtatgcataccaagattcataccggcgaaaaaccatttcaatgcaggatttgtatgcagaagttcgcctggcaatccaatttgcagaatcatactaaaattcataccggagaaaaaccattccaatgccgcatttgtatgagaaacttttctacctctggcaatctcaccagacatatcagaacacacacaggcgagaaaccgttcgcatgcgatatctgtgggcgaaagtttgccagaagatcccatctcacatcacatactaaaatacatttgcgaggaagtcaactggtcaagtccgaactggaggaaaaaaaaagtgagctgcgacacaagttgaagtacgtaccacacgaatacatcgagctgattgagatagcacggaactctacccaggatagaatactggagatgaaagttatggaattctttatgaaggtgtacggatacagggggaagcatcttggcgggagccggaaaccagacggagcaatctataccgtcgggtcacctatagactatggagttattgtcgatacaaaggcctattcaggaggttataatctgccaatcggccaagccgacgagatggagaggtacgtggaggaaaatcagaccagagacaagcacctgaaccctaatgaatggtggaaagtgtaccctagcagcgtcactgagttcaaattcctgttcgtcagcggtcattttaaaggaaattataaagcccagctcactagactcaaccatattacaaactgcgacggagccgtacttagcgttgaagagttgcttatcggaggagagatgatcaaagccggaaccctcacacttgaagaagtgcgaagaaaattcaataacggagagataaattttaggagt

SEQ ID NO: 75 - DNA, 1032 bp, Artificial Sequence
  source: /note=“Description of Artificial Sequence Synthetic polynucleotide”
gcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcgaggctcacagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagt

SEQ ID NO: 76 - DNA, 1032 bp, Artificial Sequence
  source: /note=“Description of Artificial Sequence Synthetic polynucleotide”
gcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgcggtagccaactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagt

SEQ ID NO: 77 - DNA, 1032 bp, Artificial Sequence
  source: /note=“Description of Artificial Sequence Synthetic polynucleotide”
gcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcgaggaagccagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctcc

SEQ ID NO: 78 - DNA, 1110 bp, Artificial Sequence
  source: /note=“Description of Artificial Sequence Synthetic polynucleotide”
gccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 79 - DNA, 1110 bp, Artificial Sequence
  source: /note=“Description of Artificial Sequence Synthetic polynucleotide”
gctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcagaggaagtcagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaacttt

SEQ ID NO: 80 - DNA, 1110 bp, Artificial Sequence
  source: /note=“Description of Artificial Sequence Synthetic polynucleotide”
gctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcggggttctcaactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaatttt

SEQ ID NO: 81 - DNA, 1110 bp, Artificial Sequence
  source: /note=“Description of Artificial Sequence Synthetic polynucleotide”
gccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgagaggcagtcagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaatttt

SEQ ID NO: 82 - DNA, 1110 bp, Artificial Sequence
  source: /note=“Description of Artificial Sequence Synthetic polynucleotide”
gccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttt

SEQ ID NO: 83 - DNA, 1110 bp, Artificial Sequence
  source: /note=“Description of Artificial Sequence Synthetic polynucleotide”
gcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcggggttcacaactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttc

SEQ ID NO: 84 - DNA, 1110 bp, Artificial Sequence
  source: /note=“Description of Artificial Sequence Synthetic polynucleotide”
gctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgagaggttcccagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttc

SEQ ID NO: 85 - DNA, 2439 bp, Artificial Sequence
  source: /note=“Description of Artificial Sequence Synthetic polynucleotide”
gattataaagatcatgacggggactataaggatcacgacatagactacaaagacgatgatgacaaaatggcgcctaaaaagaaacgaaaagtgggcattcacggcgtacctgctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcagaggaagtcagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaactttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 86 - DNA, 2439 bp, Artificial Sequence
  source: /note=“Description of Artificial Sequence Synthetic polynucleotide”
gattataaagaccatgatggtgattacaaggaccatgacatcgattataaagacgacgacgacaaaatggcccctaagaaaaagagaaaagtcggaatccacggtgtcccagctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcggggttctcaactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaattttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 87 - DNA, 2439 bp, Artificial Sequence
  source: /note=“Description of Artificial Sequence Synthetic polynucleotide”
gattataaggatcatgatggagactataaggatcatgacatagattacaaagatgacgatgacaagatggcacccaagaagaaaagaaaagtaggaattcacggagtccctgccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgagaggcagtcagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaattttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 88 - DNA, 2439 bp, Artificial Sequence
  source: /note=“Description of Artificial Sequence Synthetic polynucleotide”
gactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaactttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 89 - DNA, 2439 bp, Artificial Sequence
  source: /note=“Description of Artificial Sequence Synthetic polynucleotide”
gattacaaagaccatgatggcgactataaagaccatgacatcgactacaaggatgatgatgataaaatggctccaaagaaaaagaggaaggtgggaatacatggagtaccagcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcggggttcacaactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttcggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 90 - DNA, 2439 bp, Artificial Sequence
  source: /note=“Description of Artificial Sequence Synthetic polynucleotide”
gactacaaggaccacgacggagactataaagaccatgatatagattacaaggacgatgacgataaaatggcacccaaaaagaaaagaaaggtgggtattcacggagttcccgctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgagaggttcccagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttcggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 91 - DNA, 2439 bp, Artificial Sequence
  source: /note=“Description of Artificial Sequence Synthetic polynucleotide”
gactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttcggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 92 - DNA, 2439 bp, Artificial Sequence
  source: /note=“Description of Artificial Sequence Synthetic polynucleotide”
gattacaaagatcacgacggagattacaaagatcacgacattgactataaggacgacgacgataaaatggctccaaagaagaaaagaaaagtggggatccatggtgtacccgcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgcggcagccagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtctggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 93 - DNA, 2439 bp, Artificial Sequence
  source: /note=“Description of Artificial Sequence Synthetic polynucleotide”
gactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagtggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 94 - DNA, 2439 bp, Artificial Sequence
  source: /note=“Description of Artificial Sequence Synthetic polynucleotide”
gactataaagaccacgatggcgactacaaagaccacgacatcgattacaaggacgatgatgacaaaatggcacctaagaagaagagaaaagttggaatacatggagtccccgcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcgaggctcacagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagtggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 95 - DNA, 2439 bp, Artificial Sequence
  source: /note=“Description of Artificial Sequence Synthetic polynucleotide”
gattataaggaccatgacggagactataaagaccatgatattgactacaaagacgacgatgataagatggcccccaagaagaaacgaaaagtaggaatccatggcgtgcctgcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgcggtagccaactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagtggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 96 - DNA, 2439 bp, Artificial Sequence
  source: /note=“Description of Artificial Sequence Synthetic polynucleotide”
gactataaggaccatgatggagactataaagatcacgatattgactataaagatgatgatgataagatggcacctaagaagaaaagaaaggtcggcattcatggtgtgcctgcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcgaggaagccagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctccggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 97 - DNA, 2439 bp, Artificial Sequence
  source: /note=“Description of Artificial Sequence Synthetic polynucleotide”
gactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatctggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 98 - DNA, 2439 bp, Artificial Sequence
  source: /note=“Description of Artificial Sequence Synthetic polynucleotide”
gactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagtggcagcggagagggcagaggaagcctgctcacctgcggtgacgtggaggaaaaccctggccctacgcgtgccatggactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttt

SEQ ID NO: 99 - DNA, 2439 bp, Artificial Sequence
  source: /note=“Description of Artificial Sequence Synthetic polynucleotide”
gactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaactttggcagcggagagggcagaggaagcctgctcacctgcggtgacgtggaggaaaaccctggccctacgcgtgccatggactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagt

SEQ ID NO: 100 - DNA, 2211 bp, Artificial Sequence
  source: /note=“Description of Artificial Sequence Synthetic polynucleotide”
gtacctgctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcagaggaagtcagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaactttggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 101 - DNA, 2211 bp, Artificial Sequence
  source: /note=“Description of Artificial Sequence Synthetic polynucleotide”
gtcccagctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcggggttctcaactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaattttggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 102 - DNA, 2211 bp, Artificial Sequence
  source: /note=“Description of Artificial Sequence Synthetic polynucleotide”
gtccctgccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgagaggcagtcagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaattttggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 103 - DNA, 2211 bp, Artificial Sequence
  source: /note=“Description of Artificial Sequence Synthetic polynucleotide”
gtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaactttggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 104 - DNA, 2211 bp, Artificial Sequence
  source: /note=“Description of Artificial Sequence Synthetic polynucleotide”
gtaccagcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcggggttcacaactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttcggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 105 - DNA, 2211 bp, Artificial Sequence
  source: /note=“Description of Artificial Sequence Synthetic polynucleotide”
gttcccgctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgagaggttcccagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttcggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 106 - DNA, 2211 bp, Artificial Sequence
  source: /note=“Description of Artificial Sequence Synthetic polynucleotide”
gtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttcggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 107 - DNA, 2211 bp, Artificial Sequence
  source: /note=“Description of Artificial Sequence Synthetic polynucleotide”
gcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgcggcagccagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtctggcagcggagagggcagaggaagcctgctcacctgcggtgacgtggaggaaaaccctggccctgtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 108 - DNA, 2211 bp, Artificial Sequence
  source: /note=“Description of Artificial Sequence Synthetic polynucleotide”
gccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagtggcagcggagagggcagaggaagcctgctcacctgcggtgacgtggaggaaaaccctggccctgtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 109 - DNA, 2127 bp, Artificial Sequence
  source: /note=“Description of Artificial Sequence Synthetic polynucleotide”
gccgcgatggcagagagaccatttcagtgtagaatctgtatgcagaacttttcccaatcaggaaacctggcacgacacattagaacccatactggagaaaagccgttcgcttgcgacatttgcggtagaaaatttgctttgaaacagaacttgtgtatgcataccaagattcataccggcgaaaaaccatttcaatgcaggatttgtatgcagaagttcgcctggcaatccaatttgcagaatcatactaaaattcataccggagaaaaaccattccaatgccgcatttgtatgagaaacttttctacctctggcaatctcaccagacatatcagaacacacacaggcgagaaaccgttcgcatgcgatatctgtgggcgaaagtttgccagaagatcccatctcacatcacatactaaaatacatttgcgaggaagtcaactggtcaagtccgaactggaggaaaaaaaaagtgagctgcgacacaagttgaagtacgtaccacacgaatacatcgagctgattgagatagcacggaactctacccaggatagaatactggagatgaaagttatggaattctttatgaaggtgtacggatacagggggaagcatcttggcgggagccggaaaccagacggagcaatctataccgtcgggtcacctatagactatggagttattgtcgatacaaaggcctattcaggaggttataatctgccaatcggccaagccgacgagatggagaggtacgtggaggaaaatcagaccagagacaagcacctgaaccctaatgaatggtggaaagtgtaccctagcagcgtcactgagttcaaattcctgttcgtcagcggtcattttaaaggaaattataaagcccagctcactagactcaaccatattacaaactgcgacggagccgtacttagcgttgaagagttgcttatcggaggagagatgatcaaagccggaaccctcacacttgaagaagtgcgaagaaaattcaataacggagagataaattttaggagtggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 110 - DNA, 2211 bp, Artificial Sequence
  source: /note=“Description of Artificial Sequence Synthetic polynucleotide”
gcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcgaggctcacagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagtggcagcggagagggcagaggaagcctgctcacctgcggtgacgtggaggaaaaccctggccctgtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 111 - DNA, 2211 bp, Artificial Sequence
  source: /note=“Description of Artificial Sequence Synthetic polynucleotide”
gcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgcggtagccaactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagtggcagcggagagggcagaggaagcctgctcacctgcggtgacgtggaggaaaaccctggccctgtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 112 - DNA, 2211 bp, Artificial Sequence
  source: /note=“Description of Artificial Sequence Synthetic polynucleotide”
gcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcgaggaagccagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctccggcagcggagagggcagaggaagcctgctcacctgcggtgacgtggaggaaaaccctggccctgtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 113 - DNA, 2211 bp, Artificial Sequence
  source: /note=“Description of Artificial Sequence Synthetic polynucleotide”
gccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatctggcagcggagagggcagaggaagcctgctcacctgcggtgacgtggaggaaaaccctggccctgtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 114 - DNA, 2211 bp, Artificial Sequence
  source: /note=“Description of Artificial Sequence Synthetic polynucleotide”
gccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagtggcagcggagagggcagaggaagcctgctcacctgcggtgacgtggaggaaaaccctggccctgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttt

SEQ ID NO: 115 - DNA, 2211 bp, Artificial Sequence
  source: /note=“Description of Artificial Sequence Synthetic polynucleotide”
gtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaactttggcagcggagagggcagaggaagcctgctcacctgcggtgacgtggaggaaaaccctggccctgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagt

SEQ ID NO: 116 - DNA, 432 bp, Artificial Sequence
  source: /note=“Description of Artificial Sequence Synthetic polynucleotide”
gccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcgg

SEQ ID NO: 117 - DNA, 432 bp, Artificial Sequence
  source: /note=“Description of Artificial Sequence Synthetic polynucleotide”
gcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgc

SEQ ID NO: 118 - DNA, 432 bp, Artificial Sequence
  source: /note=“Description of Artificial Sequence Synthetic polynucleotide”
gccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgc

SEQ ID NO: 119 - DNA, 432 bp, Artificial Sequence
  source: /note=“Description of Artificial Sequence Synthetic polynucleotide”
gccgcgatggcagagagaccatttcagtgtagaatctgtatgcagaacttttcccaatcaggaaacctggcacgacacattagaacccatactggagaaaagccgttcgcttgcgacatttgcggtagaaaatttgctttgaaacagaacttgtgtatgcataccaagattcataccggcgaaaaaccatttcaatgcaggatttgtatgcagaagttcgcctggcaatccaatttgcagaatcatactaaaattcataccggagaaaaaccattccaatgccgcatttgtatgagaaacttttctacctctggcaatctcaccagacatatcagaacacacacaggcgagaaaccgttcgcatgcgatatctgtgggcgaaagtttgccagaagatcccatctcacatcacatactaaaatacatttgcga

SEQ ID NO: 120 - DNA, 432 bp, Artificial Sequence
  source: /note=“Description of Artificial Sequence Synthetic polynucleotide”
gcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcga

SEQ ID NO: 121 - DNA, 432 bp, Artificial Sequence
  source: /note=“Description of Artificial Sequence Synthetic polynucleotide”
gcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgc

SEQ ID NO: 122 - DNA, 432 bp, Artificial Sequence
  source: /note=“Description of Artificial Sequence Synthetic polynucleotide”
gcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcga

SEQ ID NO: 123 - DNA, 516 bp, Artificial Sequence
  source: /note=“Description of Artificial Sequence Synthetic polynucleotide”
gccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcgg

SEQ ID NO: 124 - DNA, 516 bp, Artificial Sequence
  source: /note=“Description of Artificial Sequence Synthetic polynucleotide”
gctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcaga

SEQ ID NO: 125 - DNA, 516 bp, Artificial Sequence
  source: /note=“Description of Artificial Sequence Synthetic polynucleotide”
gctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcgg

SEQ ID NO: 126 - DNA, 516 bp, Artificial Sequence
  source: /note=“Description of Artificial Sequence Synthetic polynucleotide”
gccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgaga

SEQ ID NO: 127 - DNA, 516 bp, Artificial Sequence
  source: /note=“Description of Artificial Sequence Synthetic polynucleotide”
gccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcaga

SEQ ID NO: 128 - DNA, 516 bp, Artificial Sequence
  source: /note=“Description of Artificial Sequence Synthetic polynucleotide”
gcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcgg

SEQ ID NO: 129 - DNA, 516 bp, Artificial Sequence
  source: /note=“Description of Artificial Sequence Synthetic polynucleotide”
gctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgaga

SEQ ID NO: 130 - protein, 344 aa, Artificial Sequence
  source: /note=“Description of Artificial Sequence Synthetic polypeptide”
AAMAERPFQCRICMQNFSQSGNLARHIRTHTGEKPFACDICGRKFALKQNLCMHTKIHTGEKPFQCRICMQKFAWQSNLQNHTKIHTGEKPFQCRICMRNFSTSGNLTRHIRTHTGEKPFACDICGRKFARRSHLTSHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCDGAVLSVEELLIGGEMIKAGTLTLEEVRRKFNNGEINFRS

SEQ ID NO: 131 - protein, 370 aa, Artificial Sequence
  source: /note=“Description of Artificial Sequence Synthetic polypeptide”
AAMAERPFQCRICMRNFSQSSDLSRHIRTHTGEKPFACDICGRKFALKHNLLTHTKIHTGEKPFQCRICMQNFSDQSNLRAHIRTHTGEKPFACDICGRKFARNFSLTMHTKIHTGERGFQCRICMRNFSLRHDLERHIRTHTGEKPFACDICGRKFAHRSNLNKHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLSIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 132 - protein, 813 aa, Artificial Sequence
  source: /note=“Description of Artificial Sequence Synthetic polypeptide”
DYKDHDGDYKDHDIDYKDDDDKMAPKKKRKVGIHGVPAAMAERPFQCRICMRNFSQSSDLSRHIRTHTGEKPFACDICGRKFALKHNLLTHTKIHTGEKPFQCRICMQNFSDQSNLRAHIRTHTGEKPFACDICGRKFARNFSLTMHTKIHTGERGFQCRICMRNFSLRHDLERHIRTHTGEKPFACDICGRKFAHRSNLNKHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLSIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFGSGEGRGSLLTCGDVEENPGPTRAMDYKDHDGDYKDHDIDYKDDDDKMAPKKKRKVGIHGVPAAMAERPFQCRICMQNFSQSGNLARHIRTHTGEKPFACDICGRKFALKQNLCMHTKIHTGEKPFQCRICMQKFAWQSNLQNHTKIHTGEKPFQCRICMRNFSTSGNLTRHIRTHTGEKPFACDICGRKFARRSHLTSHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCDGAVLSVEELLIGGEMIKAGTLTLEEVRRKFNNGEINFRS

SEQ ID NO: 133 - protein, 813 aa, Artificial Sequence
  source: /note=“Description of Artificial Sequence Synthetic polypeptide”
DYKDHDGDYKDHDIDYKDDDDKMAPKKKRKVGIHGVPAAMAERPFQCRICMQNFSQSGNLARHIRTHTGEKPFACDICGRKFALKQNLCMHTKIHTGEKPFQCRICMQKFAWQSNLQNHTKIHTGEKPFQCRICMRNFSTSGNLTRHIRTHTGEKPFACDICGRKFARRSHLTSHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCDGAVLSVEELLIGGEMIKAGTLTLEEVRRKFNNGEINFRSGSGEGRGSLLTCGDVEENPGPTRAMDYKDHDGDYKDHDIDYKDDDDKMAPKKKRKVGIHGVPAAMAERPFQCRICMRNFSQSSDLSRHIRTHTGEKPFACDICGRKFALKHNLLTHTKIHTGEKPFQCRICMQNFSDQSNLRAHIRTHTGEKPFACDICGRKFARNFSLTMHTKIHTGERGFQCRICMRNFSLRHDLERHIRTHTGEKPFACDICGRKFAHRSNLNKHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLSIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 134 - protein, 735 aa, Artificial Sequence
  source: /note=“Description of Artificial Sequence Synthetic polypeptide”
AAMAERPFQCRICMRNFSQSSDLSRHIRTHTGEKPFACDICGRKFALKHNLLTHTKIHTGEKPFQCRICMQNFSDQSNLRAHIRTHTGEKPFACDICGRKFARNFSLTMHTKIHTGERGFQCRICMRNFSLRHDLERHIRTHTGEKPFACDICGRKFAHRSNLNKHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLSIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFGSGEGRGSLLTCGDVEENPGPAAMAERPFQCRICMQNFSQSGNLARHIRTHTGEKPFACDICGRKFALKQNLCMHTKIHTGEKPFQCRICMQKFAWQSNLQNHTKIHTGEKPFQCRICMRNFSTSGNLTRHIRTHTGEKPFACDICGRKFARRSHLTSHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCDGAVLSVEELLIGGEMIKAGTLTLEEVRRKFNNGEINFRS

SEQ ID NO: 135 - protein, 737 aa, Artificial Sequence
  source: /note=“Description of Artificial Sequence Synthetic polypeptide”
AAMAERPFQCRICMQNFSQSGNLARHIRTHTGEKPFACDICGRKFALKQNLCMHTKIHTGEKPFQCRICMQKFAWQSNLQNHTKIHTGEKPFQCRICMRNFSTSGNLTRHIRTHTGEKPFACDICGRKFARRSHLTSHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCDGAVLSVEELLIGGEMIKAGTLTLEEVRRKFNNGEINFRSGSGEGRGSLLTCGDVEENPGPVPAAMAERPFQCRICMRNFSQSSDLSRHIRTHTGEKPFACDICGRKFALKHNLLTHTKIHTGEKPFQCRICMQNFSDQSNLRAHIRTHTGEKPFACDICGRKFARNFSLTMHTKIHTGERGFQCRICMRNFSLRHDLERHIRTHTGEKPFACDICGRKFAHRSNLNKHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLSIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 136 - protein, 144 aa, Artificial Sequence
  source: /note=“Description of Artificial Sequence Synthetic polypeptide”
AAMAERPFQCRICMQNFSQSGNLARHIRTHTGEKPFACDICGRKFALKQNLCMHTKIHTGEKPFQCRICMQKFAWQSNLQNHTKIHTGEKPFQCRICMRNFSTSGNLTRHIRTHTGEKPFACDICGRKFARRSHLTSHTKIHLR

SEQ ID NO: 137 - protein, 172 aa, Artificial Sequence
  source: /note=“Description of Artificial Sequence Synthetic polypeptide”
AAMAERPFQCRICMRNFSQSSDLSRHIRTHTGEKPFACDICGRKFALKHNLLTHTKIHTGEKPFQCRICMQNFSDQSNLRAHIRTHTGEKPFACDICGRKFARNFSLTMHTKIHTGERGFQCRICMRNFSLRHDLERHIRTHTGEKPFACDICGRKFAHRSNLNKHTKIHLR

SEQ ID NO: 138 - protein, 21 aa, Artificial Sequence
  source: /note=“Description of Artificial Sequence Synthetic peptide”
GSGEGRGSLLTCGDVEENPGP

SEQ ID NO: 139 - DNA, 1071 bp, Artificial Sequence
  source: /note=“Description of Artificial Sequence Synthetic polynucleotide”
cccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 140 - DNA, 1071 bp, Artificial Sequence
  source: /note=“Description of Artificial Sequence Synthetic polynucleotide”
ccaaagaagaaaagaaaagtggggatccatggtgtacccgcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgcggcagccagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtct

SEQ ID NO: 141 - DNA, 1071 bp, Artificial Sequence
  source: /note=“Description of Artificial Sequence Synthetic polynucleotide”
cctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagt

SEQ ID NO: 142 - DNA, 1071 bp, Artificial Sequence
  source: /note=“Description of Artificial Sequence Synthetic polynucleotide”
cccaaaaagaagagaaaagtgggaatccacggtgtaccggccgcgatggcagagagaccatttcagtgtagaatctgtatgcagaacttttcccaatcaggaaacctggcacgacacattagaacccatactggagaaaagccgttcgcttgcgacatttgcggtagaaaatttgctttgaaacagaacttgtgtatgcataccaagattcataccggcgaaaaaccatttcaatgcaggatttgtatgcagaagttcgcctggcaatccaatttgcagaatcatactaaaattcataccggagaaaaaccattccaatgccgcatttgtatgagaaacttttctacctctggcaatctcaccagacatatcagaacacacacaggcgagaaaccgttcgcatgcgatatctgtgggcgaaagtttgccagaagatcccatctcacatcacatactaaaatacatttgcgaggaagtcaactggtcaagtccgaactggaggaaaaaaaaagtgagctgcgacacaagttgaagtacgtaccacacgaatacatcgagctgattgagatagcacggaactctacccaggatagaatactggagatgaaagttatggaattctttatgaaggtgtacggatacagggggaagcatcttggcgggagccggaaaccagacggagcaatctataccgtcgggtcacctatagactatggagttattgtcgatacaaaggcctattcaggaggttataatctgccaatcggccaagccgacgagatggagaggtacgtggaggaaaatcagaccagagacaagcacctgaaccctaatgaatggtggaaagtgtaccctagcagcgtcactgagttcaaattcctgttcgtcagcggtcattttaaaggaaattataaagcccagctcactagactcaaccatattacaaactgcgacggagccgtacttagcgttgaagagttgcttatcggaggagagatgatcaaagccggaaccctcacacttgaagaagtgcgaagaaaattcaataacggagagataaattttaggagt

SEQ ID NO: 143 - DNA, 1071 bp, Artificial Sequence
  source: /note=“Description of Artificial Sequence Synthetic polynucleotide”
cctaagaagaagagaaaagttggaatacatggagtccccgcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcgaggctcacagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagt

SEQ ID NO: 144 - DNA, 1071 bp, Artificial Sequence
  source: /note=“Description of Artificial Sequence Synthetic polynucleotide”
cccaagaagaaacgaaaagtaggaatccatggcgtgcctgcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgcggtagccaactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagt

SEQ ID NO: 145 - DNA, 1071 bp, Artificial Sequence
  source: /note=“Description of Artificial Sequence Synthetic polynucleotide”
cctaagaagaaaagaaaggtcggcattcatggtgtgcctgcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcgaggaagccagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctcc

SEQ ID NO: 146 - DNA, 1149 bp, Artificial Sequence
  source: /note=“Description of Artificial Sequence Synthetic polynucleotide”
cccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 147 - DNA, 1149 bp, Artificial Sequence
  source: /note=“Description of Artificial Sequence Synthetic polynucleotide”
cctaaaaagaaacgaaaagtgggcattcacggcgtacctgctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcagaggaagtcagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaacttt

SEQ ID NO: 148 - DNA, 1149 bp, Artificial Sequence
  source: /note=“Description of Artificial Sequence Synthetic polynucleotide”
cctaagaaaaagagaaaagtcggaatccacggtgtcccagctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcggggttctcaactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaatttt

SEQ ID NO: 149 - DNA, 1149 bp, Artificial Sequence
  source: /note=“Description of Artificial Sequence Synthetic polynucleotide”
cccaagaagaaaagaaaagtaggaattcacggagtccctgccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgagaggcagtcagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaatttt

SEQ ID NO: 150 - DNA, 1149 bp, Artificial Sequence
  source: /note=“Description of Artificial Sequence Synthetic polynucleotide”
ccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttt

SEQ ID NO: 151 - DNA, 1149 bp, Artificial Sequence
  source: /note=“Description of Artificial Sequence Synthetic polynucleotide”
ccaaagaaaaagaggaaggtgggaatacatggagtaccagcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcggggttcacaactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttc

SEQ ID NO: 152 - DNA, 1149 bp, Artificial Sequence
  source: /note=“Description of Artificial Sequence Synthetic polynucleotide”
cccaaaaagaaaagaaaggtgggtattcacggagttcccgctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgagaggttcccagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttc

SEQ ID NO: 153 - DNA, 66 bp, Artificial Sequence
  source: /note=“Description of Artificial Sequence Synthetic oligonucleotide”
gattacaaagatcacgacggagattacaaagatcacgacattgactataaggacgacgacgataaa

SEQ ID NO: 154 - DNA, 66 bp, Artificial Sequence
  source: /note=“Description of Artificial Sequence Synthetic oligonucleotide”
gattacaaagaccacgacggagactacaaggaccatgatattgactacaaagacgatgatgataag

SEQ ID NO: 155 - DNA, 30 bp, Artificial Sequence
  source: /note=“Description of Artificial Sequence Synthetic oligonucleotide”
cccaaaaagaagagaaaagtgggaatccac

SEQ ID NO: 156 - protein, 10 aa, Artificial Sequence
  source: /note=“Description of Artificial Sequence Synthetic peptide”
PKKKRKVGIH

SEQ ID NO: 157 - DNA, 588 bp, Artificial Sequence
  source: /note=“Description of Artificial Sequence Synthetic polynucleotide”
cagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 158 - DNA, 588 bp, Artificial Sequence
  source: /note=“Description of Artificial Sequence Synthetic polynucleotide”
cagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaacttt

SEQ ID NO: 159 - DNA, 588 bp, Artificial Sequence
  source: /note=“Description of Artificial Sequence Synthetic polynucleotide”
caactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaatttt

SEQ ID NO: 160 - DNA, 588 bp, Artificial Sequence
  source: /note=“Description of Artificial Sequence Synthetic polynucleotide”
cagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaatttt

SEQ ID NO: 161 - DNA, 588 bp, Artificial Sequence
  source: /note=“Description of Artificial Sequence Synthetic polynucleotide”
cagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttt

SEQ ID NO: 162 - DNA, 588 bp, Artificial Sequence
  source: /note=“Description of Artificial Sequence Synthetic polynucleotide”
caactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttc

SEQ ID NO: 163 - DNA, 588 bp, Artificial Sequence
  source: /note=“Description of Artificial Sequence Synthetic polynucleotide”
cagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttc

SEQ ID NO: 164 - DNA, 594 bp, Artificial Sequence
  source: /note=“Description of Artificial Sequence Synthetic polynucleotide”
cagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 165 - DNA, 594 bp, Artificial Sequence
  source: /note=“Description of Artificial Sequence Synthetic polynucleotide”
cagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtct

SEQ ID NO: 166 - DNA, 594 bp, Artificial Sequence
  source: /note=“Description of Artificial Sequence Synthetic polynucleotide”
cagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagt

SEQ ID NO: 167 - DNA, 594 bp, Artificial Sequence
  source: /note=“Description of Artificial Sequence Synthetic polynucleotide”
caactggtcaagtccgaactggaggaaaaaaaaagtgagctgcgacacaagttgaagtacgtaccacacgaatacatcgagctgattgagatagcacggaactctacccaggatagaatactggagatgaaagttatggaattctttatgaaggtgtacggatacagggggaagcatcttggcgggagccggaaaccagacggagcaatctataccgtcgggtcacctatagactatggagttattgtcgatacaaaggcctattcaggaggttataatctgccaatcggccaagccgacgagatggagaggtacgtggaggaaaatcagaccagagacaagcacctgaaccctaatgaatggtggaaagtgtaccctagcagcgtcactgagttcaaattcctgttcgtcagcggtcattttaaaggaaattataaagcccagctcactagactcaaccatattacaaactgcgacggagccgtacttagcgttgaagagttgcttatcggaggagagatgatcaaagccggaaccctcacacttgaagaagtgcgaagaaaattcaataacggagagataaattttaggagt

SEQ ID NO: 168 - DNA, 594 bp, Artificial Sequence
  source: /note=“Description of Artificial Sequence Synthetic polynucleotide”
cagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagt

SEQ ID NO: 169 - DNA, 594 bp, Artificial Sequence
  source: /note=“Description of Artificial Sequence Synthetic polynucleotide”
caactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagt

SEQ ID NO: 170 - DNA, 594 bp, Artificial Sequence
  source: /note=“Description of Artificial Sequence Synthetic polynucleotide”
cagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctcc

SEQ ID NO: 171 - protein, 196 aa, Artificial Sequence
  source: /note=“Description of Artificial Sequence Synthetic polypeptide”
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLSIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 172 - protein, 198 aa, Artificial Sequence
  source: /note=“Description of Artificial Sequence Synthetic polypeptide”
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCDGAVLSVEELLIGGEMIKAGTLTLEEVRRKFNNGEINFRS

SEQ ID NO: 173 - DNA, 4341 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttactaaagaattattcttttacatttcagttagtgagacgcaggctaactccaccactgatgcattgaacgtcctccttatcattgttgacgatcttcgaccctctttgggctgctacggcgacaaactggttcgcagccccaacatagaccagcttgcttcccattcactgctttttcagaacgcgtttgctcagcaagccgtctgcgcaccatcccgcgtttcttttcttactggacgacgccctgacacgacccgactgtacgattttaatagttactggcgcgttcatgccggcaatttctcaaccatccctcagtacttcaaagagaacggatacgtcaccatgagcgttggcaaggtgttccatccaggcatctcttccaaccataccgacgatagcccatacagctggtcctttcccccatatcatccctcaagtgaaaaatatgaaaatacaaagacatgcagaggtcccgacggcgagcttcacgccaatctcctgtgtccagttgatgtgctcgatgtgccagaggggacactccctgataaacaatctactgagcaggctatccagctccttgagaaaatgaaaacctctgccagcccctttttcttggccgtcggttaccacaagccccacattccattccggtatccaaaagaattccagaaattgtatcctcttgaaaacatcaccctggcccccgaccctgaagtgcccgatggcctgccccctgtcgcctataacccatggatggatatcaggcagagagaggacgtgcaggcccttaatatctcagttccctacggaccaattcccgttgattttcaaagaaagatccgccagtcctactttgctagcgtctcatacctcgacacacaggtcggcagacttctcagcgccctcgacgacctgcaattggctaacagcaccatcattgccttcacctctgaccacgggtgggcgctcggcgaacacggcgagtgggccaaatattcaaatttcgacgtcgccacacacgtaccccttatcttttacgtccccggtagaaccgctagtctgcccgaagcaggagagaaactgttcccctatctggacccctttgattcagctagccaattgatggagcccggtagacaatccatggatttggttgaactcgtgtccctctttcccacgctggccggtctggccggtctccaagttccccccaggtgccccgttccttctttccacgtagagctgtgcagggagggaaaaaacttgcttaaacattttcggtttcgcgacctggaggaagacccctacttgcccggtaatccccgcgagctgatcgcttattcccaataccctagacctagcgacatccctcagtggaattccgataagccgtccctcaaggacattaagattatgggatactctattcgcactattgactacagatataccgtctgggtgggcttcaatcctgatgaattcctggcaaacttttccgatattcacgctggtgagctgtatttcgtcgactccgatccactgcaagaccacaatatgtacaacgattcccaaggcggagatttgttccagctcttgatgccttgataaagatctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccacccgttctagattatcacggcatgagcagctggaacaaatctcctccttgggaatcattatacatattgtgatcttgcaacgggtccgagtctacgaaatacagctcaccagcgtggatgtccgaaaagttcgcgaggaattcgtcaggattgaaccctacccacactgtgtagcgatagtcgatggtcctgatcgagtaccccataatcttgatgtctttgagggagggcttatcggagttccattgaggaatatcgctgggtcgcggatactgggaataggcaatcaactctcgcggattccctggcagataggggtcctcctcaaggtccctgaaccgaaagtgtttgaggaggtttttcccttcgcggcagagttccacatggaagctcggtacagggcatctagggggtacttgcaagcccgccaacccggcgagggtcggaaaaagggacaccaattctaccaagtccatggattgtctgcccggttccataagctggctcgccgagtcgaatggatcgagatagggaaaaagtttttcgcctgcctcgggaagcgaggccgttctacccggcacgtagaaaatcaggggcacgtgcgttgctacatcaaaattgctatactttgcccactctccatgctctcccaacgcccacccatggtccgacgtaaaggcgatgattgtggaatttgccagctgaaggtcatcaagcgcgctcagaagtcgacctacttgcgtatcgaggtaggacaccgacgcaaaatacgactgccgaatcttgcgttgaaaatcgactggaataggcccgtaggggactgagatgttgagtgcctgcacatcttccctctgcctgatatccatccagggattgtaggccacgggtggcagaccgtcggggacttccgggtccggtgccaaagtgatgttttccaaaggataaagtttctggaactccttcgggtagcggaaaggaatatggggcttgtgataccccacggcgaggaagaaaggcgacgcgcttgttttcatcttctccagcaactgaatcgcctgctccgttgactgcttgtcggggagcgttccctcgggcacgtccaagacatccaccggacacagcagattagcgtgcagctctccgtcgggtccgcgacaagttttcgtgttctcatacttctcgctcgaaggatggtagggaggaaacgaccacgagtagggcgaatcgtcggtgtgattcgaggagatgccggggtgaaagacctttcccacgctcattgtcacgtatccgttctctttaaagtactgtgggatagttgaaaagttacccgcgtggactctccagtagctgttgaagtcgtacagccgcgttgtgtcagggcgtcgcccggtcaagaatgagactcttgaaggtgcacagacagcctgctgcgcaaacgcattttggaaaagcagtgagtgtgaggccaactgatcgatgttcggcgagcggacgagcttatctccatagcagccaagcgacggccgcaaatcgtccacgatgatgagcaggacgttaagcgcatctgtagttgagttggcctgggtttcgctaactgaaatgtaaaagaataattctttagtggatccacaaattaatcgaacctgcagctgatatcgacgcttaagtagggcttagcaaacgcgtctccaacgtttcgccgttaacaccccacatagtgagtggtcttagtagtccgggtgtttaaactgaaagataactcgagcgcaggaacccctagtgatggagttggccactccctctctgcgcgctcgctcgctcactgaggccgcccgggctttgcccgggcggcctcagtgagcgagcgagcgcgcag

SEQ ID NO: 174 - DNA, 4341 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttactaaagaattattcttttacatttcagtttcagagactcaagcaaatagcactacggacgccttgaatgttttgctgattatagtggatgacctcagaccttcactcggctgttacggtgacaaactggtccgctctccgaatatcgaccaactggcaagccactccctccttttccaaaacgcattcgctcaacaagcagtttgtgcccccagtagagtgtccttcttgactggtcgcaggcccgacaccacccgcctgtacgattttaactcatattggcgcgttcatgccggcaacttttctacaataccacaatactttaaggaaaatggctacgtaactatgagtgtgggcaaggtgtttcaccccggtatttcaagcaatcacacagacgactctccctactcctggtcctttcccccataccatccttcctcagagaagtacgaaaataccaagacgtgtagaggtccggacggcgaactgcacgcaaacctgttgtgccctgttgacgtactcgacgtcccggaaggcaccctccccgacaagcaatctaccgagcaggccattcagctcctcgaaaagatgaaaacaagtgcatccccctttttcctggctgtaggttatcataaaccccacattccattccggtatcctaaagaatttcagaagctgtacccccttgaaaacattacactggcaccagacccagaagtcccagacggactccccccagtggcctataacccatggatggacatcaggcagcgcgaagacgtgcaggctcttaacatcagcgtcccatatggcccaatacctgtcgactttcaacgcaagattagacaatcctatttcgcttctgtgagttacctggacacacaagtaggaagactgctcagcgcccttgacgatctgcaactcgctaattctaccataattgcctttaccagcgaccatggatgggcactcggagaacacggcgaatgggcaaagtactccaatttcgatgtcgcaacccacgttcccttgatattctatgtccccggccgcactgcgtccttgccagaagctggggaaaaactctttccatatctggaccccttcgactctgcatcccaactgatggaacccggtagacaaagtatggatctggtcgagctcgtttcactctttccgacccttgccggtctcgccggccttcaggtgccaccacgatgccccgttccgagcttccacgtcgagctttgtagagaagggaaaaacctcctgaaacatttccgatttcgcgacctggaggaagacccatacctgcccgggaatcctagagaactcatcgcatattctcagtaccccagaccctccgacatcccacagtggaactctgacaaaccatctttgaaagacattaagattatgggctacagcatccggactatagattacaggtataccgtatgggttggattcaatcccgatgaattcctcgcgaatttctcagacatccacgcaggagaactctatttcgtggactcagacccccttcaagatcacaacatgtacaacgattcccaaggaggtgatctttttcagttgctcatgccttgataaagatctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccacccgttctagattatcacggcatgagcagctggaacaaatctcctccttgggaatcattatacatattgtgatcttgcaacgggtccgagtctacgaaatacagctcaccagcgtggatgtccgaaaagttcgcgaggaattcgtcaggattgaaccctacccacactgtgtagcgatagtcgatggtcctgatcgagtaccccataatcttgatgtctttgagggagggcttatcggagttccattgaggaatatcgctgggtcgcggatactgggaataggcaatcaactctcgcggattccctggcagataggggtcctcctcaaggtccctgaaccgaaagtgtttgaggaggtttttcccttcgcggcagagttccacatggaagctcggtacagggcatctagggggtacttgcaagcccgccaacccggcgagggtcggaaaaagggacaccaattctaccaagtccatggattgtctgcccggttccataagctggctcgccgagtcgaatggatcgagatagggaaaaagtttttcgcctgcctcgggaagcgaggccgttctacccggcacgtagaaaatcaggggcacgtgcgttgctacatcaaaattgctatactttgcccactctccatgctctcccaacgcccacccatggtccgacgtaaaggcgatgattgtggaatttgccagctgaaggtcatcaagcgcgctcagaagtcgacctacttgcgtatcgaggtaggacaccgacgcaaaatacgactgccgaatcttgcgttgaaaatcgactggaataggcccgtaggggactgagatgttgagtgcctgcacatcttccctctgcctgatatccatccagggattgtaggccacgggtggcagaccgtcggggacttccgggtccggtgccaaagtgatgttttccaaaggataaagtttctggaactccttcgggtagcggaaaggaatatggggcttgtgataccccacggcgaggaagaaaggcgacgcgcttgttttcatcttctccagcaactgaatcgcctgctccgttgactgcttgtcggggagcgttccctcgggcacgtccaagacatccaccggacacagcagattagcgtgcagctctccgtcgggtccgcgacaagttttcgtgttctcatacttctcgctcgaaggatggtagggaggaaacgaccacgagtagggcgaatcgtcggtgtgattcgaggagatgccggggtgaaagacctttcccacgctcattgtcacgtatccgttctctttaaagtactgtgggatagttgaaaagttacccgcgtggactctccagtagctgttgaagtcgtacagccgcgttgtgtcagggcgtcgcccggtcaagaatgagactcttgaaggtgcacagacagcctgctgcgcaaacgcattttggaaaagcagtgagtgtgaggccaactgatcgatgttcggcgagcggacgagcttatctccatagcagccaagcgacggccgcaaatcgtccacgatgatgagcaggacgttaagcgcatctgtagttgagttggcctgggtttcgctaactgaaatgtaaaagaataattctttagtggatccacaaattaatcgaacctgcagctgatatcgacgcttaagtagggcttagcaaacgcgtctccaacgtttcgccgttaacaccccacatagtgagtggtcttagtagtccgggtgtttaaactgaaagataactcgagcgcaggaacccctagtgatggagttggccactccctctctgcgcgctcgctcgctcactgaggccgcccgggctttgcccgggcggcctcagtgagcgagcgagcgcgcag

SEQ ID NO: 175 - DNA, 4341 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttactaaagaattattcttttacatttcagttagtgaaacgcaggcgaactcaaccaccgatgcgctgaacgttctgcttattatcgtggatgatctgcgaccctcacttggttgctatggcgataaattggttagaagtccgaacatagaccagctggcgagtcattctctcctcttccaaaacgcgttcgcacaacaggccgtttgcgccccttcaagagtatcctttctgacaggcagacgccccgatactactaggctgtatgacttcaattcctactggcgcgtgcacgcaggtaatttctctacaatcccccagtacttcaaagaaaacggatacgttaccatgagcgtcggcaaagtgttccatcccggaatttctagcaaccatacggatgacagcccctattcctggtcatttccaccgtaccatccttccagtgaaaaatatgagaacactaaaacttgtcgcggacctgacggagaattgcacgcaaaccttctctgccccgtagatgtgctcgatgtgcctgaaggaactctcccagacaagcagagtaccgaacaagccattcagctgctggaaaagatgaaaacgtccgcctcacctttcttcctcgcagtcggttaccacaagccccacattccttttagataccctaaagagtttcagaaactgtatccccttgaaaatatcaccctcgctcccgaccccgaggtcccggacggcctgccccctgttgcatacaacccctggatggatatcagacaacgggaggatgttcaagcactcaacatctcagtaccatacggcccaatccctgtcgatttccaaaggaaaatcaggcagtcctactttgcaagcgtgtcttatctcgacacccaggtcggaagactgctgtccgccctcgacgaccttcaattggctaactctacaatcattgccttcactagcgatcacgggtgggcgcttggcgagcacggagaatgggccaaatactctaattttgatgttgccacccacgtgcccctcatattttatgttccaggtagaaccgcaagcctgccagaagccggtgagaagctgtttccttacctcgatcctttcgatagtgcatcccaactgatggagccaggtcgacaatctatggacctggtagagctggtctctctgttcccaacgctcgccggacttgctggactgcaggtgccaccccgctgccctgtaccctccttccacgttgagctctgccgcgaaggcaagaacctgttgaaacattttcgattcagagaccttgaagaggacccatacctcccaggaaatccaagagagctgattgcttattctcaatatcccaggcccagtgacataccacagtggaatagcgataaaccctcacttaaagacattaagataatgggctattccatccggacaattgattacagatacacagtttgggtggggtttaacccagacgaattccttgcgaatttcagcgatattcatgccggagaactttattttgttgatagcgaccccctccaggaccacaacatgtacaacgactcacagggtggcgatctctttcagctcctgatgccgtgataaagatctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccacccgttctagattatcacggcatgagcagctggaacaaatctcctccttgggaatcattatacatattgtgatcttgcaacgggtccgagtctacgaaatacagctcaccagcgtggatgtccgaaaagttcgcgaggaattcgtcaggattgaaccctacccacactgtgtagcgatagtcgatggtcctgatcgagtaccccataatcttgatgtctttgagggagggcttatcggagttccattgaggaatatcgctgggtcgcggatactgggaataggcaatcaactctcgcggattccctggcagataggggtcctcctcaaggtccctgaaccgaaagtgtttgaggaggtttttcccttcgcggcagagttccacatggaagctcggtacagggcatctagggggtacttgcaagcccgccaacccggcgagggtcggaaaaagggacaccaattctaccaagtccatggattgtctgcccggttccataagctggctcgccgagtcgaatggatcgagatagggaaaaagtttttcgcctgcctcgggaagcgaggccgttctacccggcacgtagaaaatcaggggcacgtgcgttgctacatcaaaattgctatactttgcccactctccatgctctcccaacgcccacccatggtccgacgtaaaggcgatgattgtggaatttgccagctgaaggtcatcaagcgcgctcagaagtcgacctacttgcgtatcgaggtaggacaccgacgcaaaatacgactgccgaatcttgcgttgaaaatcgactggaataggcccgtaggggactgagatgttgagtgcctgcacatcttccctctgcctgatatccatccagggattgtaggccacgggtggcagaccgtcggggacttccgggtccggtgccaaagtgatgttttccaaaggataaagtttctggaactccttcgggtagcggaaaggaatatggggcttgtgataccccacggcgaggaagaaaggcgacgcgcttgttttcatcttctccagcaactgaatcgcctgctccgttgactgcttgtcggggagcgttccctcgggcacgtccaagacatccaccggacacagcagattagcgtgcagctctccgtcgggtccgcgacaagttttcgtgttctcatacttctcgctcgaaggatggtagggaggaaacgaccacgagtagggcgaatcgtcggtgtgattcgaggagatgccggggtgaaagacctttcccacgctcattgtcacgtatccgttctctttaaagtactgtgggatagttgaaaagttacccgcgtggactctccagtagctgttgaagtcgtacagccgcgttgtgtcagggcgtcgcccggtcaagaatgagactcttgaaggtgcacagacagcctgctgcgcaaacgcattttggaaaagcagtgagtgtgaggccaactgatcgatgttcggcgagcggacgagcttatctccatagcagccaagcgacggccgcaaatcgtccacgatgatgagcaggacgttaagcgcatctgtagttgagttggcctgggtttcgctaactgaaatgtaaaagaataattctttagtggatccacaaattaatcgaacctgcagctgatatcgacgcttaagtagggcttagcaaacgcgtctccaacgtttcgccgttaacaccccacatagtgagtggtcttagtagtccgggtgtttaaactgaaagataactcgagcgcaggaacccctagtgatggagttggccactccctctctgcgcgctcgctcgctcactgaggccgcccgggctttgcccgggcggcctcagtgagcgagcgagcgcgcag

SEQ ID NO: 176 - DNA, 4341 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttactaaagaattattcttttacatttcagtttctgaaacccaggctaactctacgaccgacgcattgaatgttctgcttatcattgtagatgaccttcgccccagtttgggatgttatggcgataagctggtgcgctcacctaatattgatcagttggcaagccatagccttttgttccaaaatgcttttgctcagcaggctgtatgtgcaccgagtagagtttccttcctcaccggacgcagaccagacaccacaagactctacgactttaactcatactggagggtccacgctgggaatttcagtacgatcccgcagtatttcaaagaaaatggctacgttaccatgtccgtcggcaaggtgtttcaccccggcatctcatcaaatcatacagacgatagcccttattcttggtctttccctccttatcatccatccagcgaaaaatacgagaacactaaaacatgtagaggtccagatggagagctgcacgccaacctgctgtgccctgtggatgttctcgacgtacctgaaggcacccttccagacaaacagagcaccgaacaggccatccagcttctggagaagatgaagaccagcgcctcacctttcttcctcgccgtaggctaccacaaaccgcacatcccctttagatacccaaaggaatttcagaagctgtaccccctggaaaatataacattggctccagacccggaagtgcccgatgggttgccccccgtagcctataatccttggatggatattagacaacgggaagacgtccaggccctcaatatttctgtcccttacggaccaatccctgttgattttcagagaaagataagacagtcctattttgcaagtgtatcctaccttgacacccaggtcggccggctgttgtctgctctggacgacctgcaactcgctaacagtacaatcatagcctttactagcgaccacggatgggctctgggagaacatggagaatgggccaagtattctaacttcgatgtcgccacacacgtcccactcatattttacgttcctggtcgaaccgctagcctgcctgaagccggagaaaagctgtttccttatctcgaccctttcgattccgcaagccagttgatggaacccggccggcaatcaatggatctcgtggaactggtgtcactttttcctacactcgctggactcgctggccttcaagtccctccccgatgtcctgtcccatcatttcacgtagagctgtgtagagaagggaagaatctgctgaaacacttccggttccgggatcttgaagaagatccatatctcccaggcaaccctcgcgaactcatcgcttatagccagtatcctcggcccagtgacataccccagtggaattccgacaaaccatcacttaaagatatcaaaattatgggatactccattcgaaccatagactataggtacaccgtgtgggttggctttaatccagatgagtttttggcaaacttttcagacattcacgccggagagctgtattttgtggacagtgaccctctgcaggaccataatatgtacaacgattcacaaggcggcgacctcttccaactgctgatgccctgataaagatctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccacccgttctagattatcacggcatgagcagctggaacaaatctcctccttgggaatcattatacatattgtgatcttgcaacgggtccgagtctacgaaatacagctcaccagcgtggatgtccgaaaagttcgcgaggaattcgtcaggattgaaccctacccacactgtgtagcgatagtcgatggtcctgatcgagtaccccataatcttgatgtctttgagggagggcttatcggagttccattgaggaatatcgctgggtcgcggatactgggaataggcaatcaactctcgcggattccctggcagataggggtcctcctcaaggtccctgaaccgaaagtgtttgaggaggtttttcccttcgcggcagagttccacatggaagctcggtacagggcatctagggggtacttgcaagcccgccaacccggcgagggtcggaaaaagggacaccaattctaccaagtccatggattgtctgcccggttccataagctggctcgccgagtcgaatggatcgagatagggaaaaagtttttcgcctgcctcgggaagcgaggccgttctacccggcacgtagaaaatcaggggcacgtgcgttgctacatcaaaattgctatactttgcccactctccatgctctcccaacgcccacccatggtccgacgtaaaggcgatgattgtggaatttgccagctgaaggtcatcaagcgcgctcagaagtcgacctacttgcgtatcgaggtaggacaccgacgcaaaatacgactgccgaatcttgcgttgaaaatcgactggaataggcccgtaggggactgagatgttgagtgcctgcacatcttccctctgcctgatatccatccagggattgtaggccacgggtggcagaccgtcggggacttccgggtccggtgccaaagtgatgttttccaaaggataaagtttctggaactccttcgggtagcggaaaggaatatggggcttgtgataccccacggcgaggaagaaaggcgacgcgcttgttttcatcttctccagcaactgaatcgcctgctccgttgactgcttgtcggggagcgttccctcgggcacgtccaagacatccaccggacacagcagattagcgtgcagctctccgtcgggtccgcgacaagttttcgtgttctcatacttctcgctcgaaggatggtagggaggaaacgaccacgagtagggcgaatcgtcggtgtgattcgaggagatgccggggtgaaagacctttcccacgctcattgtcacgtatccgttctctttaaagtactgtgggatagttgaaaagttacccgcgtggactctccagtagctgttgaagtcgtacagccgcgttgtgtcagggcgtcgcccggtcaagaatgagactcttgaaggtgcacagacagcctgctgcgcaaacgcattttggaaaagcagtgagtgtgaggccaactgatcgatgttcggcgagcggacgagcttatctccatagcagccaagcgacggccgcaaatcgtccacgatgatgagcaggacgttaagcgcatctgtagttgagttggcctgggtttcgctaactgaaatgtaaaagaataattctttagtggatccacaaattaatcgaacctgcagctgatatcgacgcttaagtagggcttagcaaacgcgtctccaacgtttcgccgttaacaccccacatagtgagtggtcttagtagtccgggtgtttaaactgaaagataactcgagcgcaggaacccctagtgatggagttggccactccctctctgcgcgctcgctcgctcactgaggccgcccgggctttgcccgggcggcctcagtgagcgagcgagcgcgcag

SEQ ID NO: 177 - DNA, 130 bp, Artificial Sequence; identical to SEQ ID NO: 10
  source: /note=“Description of Artificial Sequence Synthetic polynucleotide”

SEQ ID NO: 178 - DNA, 28 bp, Artificial Sequence
  source: /note=“Description of Artificial Sequence Synthetic oligonucleotide”
actaaagaattattcttttacatttcag

SEQ ID NO: 179 - DNA, 225 bp, Artificial Sequence
  source: /note=“Description of Artificial Sequence Synthetic polynucleotide”
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 180 - DNA, 479 bp, Artificial Sequence
  source: /note=“Description of Artificial Sequence Synthetic polynucleotide”
cctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgatgccacccgt

SEQ ID NO: 181 - DNA, 108 bp, Artificial Sequence; identical to SEQ ID NO: 34
  source: /note=“Description of Artificial Sequence Synthetic polynucleotide”

SEQ ID NO: 182 - DNA, 28 bp, Artificial Sequence
  source: /note=“Description of Artificial Sequence Synthetic oligonucleotide”
ctgaaatgtaaaagaataattctttagt

SEQ ID NO: 183 - DNA, 479 bp, Artificial Sequence
  source: /note=“Description of Artificial Sequence Synthetic polynucleotide”
acgggtggcatcaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagg

SEQ ID NO: 184 - DNA, 1578 bp, Artificial Sequence
  source: /note=“Description of Artificial Sequence Synthetic polynucleotide”
agtgagacgcaggctaactccaccactgatgcattgaacgtcctccttatcattgttgacgatcttcgaccctctttgggctgctacggcgacaaactggttcgcagccccaacatagaccagcttgcttcccattcactgctttttcagaacgcgtttgctcagcaagccgtctgcgcaccatcccgcgtttcttttcttactggacgacgccctgacacgacccgactgtacgattttaatagttactggcgcgttcatgccggcaatttctcaaccatccctcagtacttcaaagagaacggatacgtcaccatgagcgttggcaaggtgttccatccaggcatctcttccaaccataccgacgatagcccatacagctggtcctttcccccatatcatccctcaagtgaaaaatatgaaaatacaaagacatgcagaggtcccgacggcgagcttcacgccaatctcctgtgtccagttgatgtgctcgatgtgccagaggggacactccctgataaacaatctactgagcaggctatccagctccttgagaaaatgaaaacctctgccagcccctttttcttggccgtcggttaccacaagccccacattccattccggtatccaaaagaattccagaaattgtatcctcttgaaaacatcaccctggcccccgaccctgaagtgcccgatggcctgccccctgtcgcctataacccatggatggatatcaggcagagagaggacgtgcaggcccttaatatctcagttccctacggaccaattcccgttgattttcaaagaaagatccgccagtcctactttgctagcgtctcatacctcgacacacaggtcggcagacttctcagcgccctcgacgacctgcaattggctaacagcaccatcattgccttcacctctgaccacgggtgggcgctcggcgaacacggcgagtgggccaaatattcaaatttcgacgtcgccacacacgtaccccttatcttttacgtccccggtagaaccgctagtctgcccgaagcaggagagaaactgttcccctatctggacccctttgattcagctagccaattgatggagcccggtagacaatccatggatttggttgaactcgtgtccctctttcccacgctggccggtctggccggtctccaagttccccccaggtgccccgttccttctttccacgtagagctgtgcagggagggaaaaaacttgcttaaacattttcggtttcgcgacctggaggaagacccctacttgcccggtaatccccgcgagctgatcgcttattcccaataccctagacctagcgacatccctcagtggaattccgataagccgtccctcaaggacattaagattatgggatactctattcgcactattgactacagatataccgtctgggtgggcttcaatcctgatgaattcctggcaaacttttccgatattcacgctggtgagctgtatttcgtcgactccgatccactgcaagaccacaatatgtacaacgattcccaaggcggagatttgttccagctcttgatgccttga

SEQ ID NO: 185 - DNA, 1578 bp, Artificial Sequence
  source: /note=“Description of Artificial Sequence Synthetic polynucleotide”
agcgaaacccaggccaactcaactacagatgcgcttaacgtcctgctcatcatcgtggacgatttgcggccgtcgcttggctgctatggagataagctcgtccgctcgccgaacatcgatcagttggcctcacactcactgcttttccaaaatgcgtttgcgcagcaggctgtctgtgcaccttcaagagtctcattcttgaccgggcgacgccctgacacaacgcggctgtacgacttcaacagctactggagagtccacgcgggtaacttttcaactatcccacagtactttaaagagaacggatacgtgacaatgagcgtgggaaaggtctttcaccccggcatctcctcgaatcacaccgacgattcgccctactcgtggtcgtttcctccctaccatccttcgagcgagaagtatgagaacacgaaaacttgtcgcggacccgacggagagctgcacgctaatctgctgtgtccggtggatgtcttggacgtgcccgagggaacgctccccgacaagcagtcaacggagcaggcgattcagttgctggagaagatgaaaacaagcgcgtcgcctttcttcctcgccgtggggtatcacaagccccatattcctttccgctacccgaaggagttccagaaactttatcctttggaaaacatcactttggcaccggacccggaagtccccgacggtctgccacccgtggcctacaatccctggatggatatcaggcagagggaagatgtgcaggcactcaacatctcagtcccctacgggcctattccagtcgattttcaacgcaagattcggcagtcgtattttgcgtcggtgtcctacctcgatacgcaagtaggtcgacttctgagcgcgcttgatgaccttcagctggcaaattccacaatcatcgcctttacgtcggaccatgggtgggcgttgggagagcatggagagtgggcaaagtatagcaattttgatgtagcaacgcacgtgcccctgattttctacgtgccgggtagaacggcctcgcttcccgaggcaggcgaaaaactttttccctatctcgatccattcgactcggcgagccagcttatggaaccgggcagacaatccatggacttggtagaattggtgtccctttttccgaccctcgccgggttggcgggcttgcaagtaccccctagatgccctgtaccgagcttccatgtggaactctgccgcgaagggaaaaacctcctcaaacactttcggttcagggaccttgaggaggacccctatctgccagggaatccgcgagagttgattgcctattcccagtatccgcgacccagcgatattcctcaatggaactccgataagccctccctcaaagacatcaagattatggggtactcgatcaggaccatcgactatcgctacacagtgtgggtagggttcaatcctgacgaattcctcgcgaacttttcggacatccacgctggtgagctgtatttcgtagactcggacccgttgcaagatcacaatatgtataatgattcccaaggaggagatttgttccagctgctcatgccgtga

SEQ ID NO: 186 - DNA, 1578 bp, Artificial Sequence
  source: /note=“Description of Artificial Sequence Synthetic polynucleotide”
tcagagactcaagcaaatagcactacggacgccttgaatgttttgctgattatagtggatgacctcagaccttcactcggctgttacggtgacaaactggtccgctctccgaatatcgaccaactggcaagccactccctccttttccaaaacgcattcgctcaacaagcagtttgtgcccccagtagagtgtccttcttgactggtcgcaggcccgacaccacccgcctgtacgattttaactcatattggcgcgttcatgccggcaacttttctacaataccacaatactttaaggaaaatggctacgtaactatgagtgtgggcaaggtgtttcaccccggtatttcaagcaatcacacagacgactctccctactcctggtcctttcccccataccatccttcctcagagaagtacgaaaataccaagacgtgtagaggtccggacggcgaactgcacgcaaacctgttgtgccctgttgacgtactcgacgtcccggaaggcaccctccccgacaagcaatctaccgagcaggccattcagctcctcgaaaagatgaaaacaagtgcatccccctttttcctggctgtaggttatcataaaccccacattccattccggtatcctaaagaatttcagaagctgtacccccttgaaaacattacactggcaccagacccagaagtcccagacggactccccccagtggcctataacccatggatggacatcaggcagcgcgaagacgtgcaggctcttaacatcagcgtcccatatggcccaatacctgtcgactttcaacgcaagattagacaatcctatttcgcttctgtgagttacctggacacacaagtaggaagactgctcagcgcccttgacgatctgcaactcgctaattctaccataattgcctttaccagcgaccatggatgggcactcggagaacacggcgaatgggcaaagtactccaatttcgatgtcgcaacccacgttcccttgatattctatgtccccggccgcactgcgtccttgccagaagctggggaaaaactctttccatatctggaccccttcgactctgcatcccaactgatggaacccggtagacaaagtatggatctggtcgagctcgtttcactctttccgacccttgccggtctcgccggccttcaggtgccaccacgatgccccgttccgagcttccacgtcgagctttgtagagaagggaaaaacctcctgaaacatttccgatttcgcgacctggaggaagacccatacctgcccgggaatcctagagaactcatcgcatattctcagtaccccagaccctccgacatcccacagtggaactctgacaaaccatctttgaaagacattaagattatgggctacagcatccggactatagattacaggtataccgtatgggttggattcaatcccgatgaattcctcgcgaatttctcagacatccacgcaggagaactctatttcgtggactcagacccccttcaagatcacaacatgtacaacgattcccaaggaggtgatctttttcagttgctcatgccttga

SEQ ID NO: 187 - DNA, 1578 bp, Artificial Sequence
  source: /note=“Description of Artificial Sequence Synthetic polynucleotide”
agtgaaacgcaggcgaactcaaccaccgatgcgctgaacgttctgcttattatcgtggatgatctgcgaccctcacttggttgctatggcgataaattggttagaagtccgaacatagaccagctggcgagtcattctctcctcttccaaaacgcgttcgcacaacaggccgtttgcgccccttcaagagtatcctttctgacaggcagacgccccgatactactaggctgtatgacttcaattcctactggcgcgtgcacgcaggtaatttctctacaatcccccagtacttcaaagaaaacggatacgttaccatgagcgtcggcaaagtgttccatcccggaatttctagcaaccatacggatgacagcccctattcctggtcatttccaccgtaccatccttccagtgaaaaatatgagaacactaaaacttgtcgcggacctgacggagaattgcacgcaaaccttctctgccccgtagatgtgctcgatgtgcctgaaggaactctcccagacaagcagagtaccgaacaagccattcagctgctggaaaagatgaaaacgtccgcctcacctttcttcctcgcagtcggttaccacaagccccacattccttttagataccctaaagagtttcagaaactgtatccccttgaaaatatcaccctcgctcccgaccccgaggtcccggacggcctgccccctgttgcatacaacccctggatggatatcagacaacgggaggatgttcaagcactcaacatctcagtaccatacggcccaatccctgtcgatttccaaaggaaaatcaggcagtcctactttgcaagcgtgtcttatctcgacacccaggtcggaagactgctgtccgccctcgacgaccttcaattggctaactctacaatcattgccttcactagcgatcacgggtgggcgcttggcgagcacggagaatgggccaaatactctaattttgatgttgccacccacgtgcccctcatattttatgttccaggtagaaccgcaagcctgccagaagccggtgagaagctgtttccttacctcgatcctttcgatagtgcatcccaactgatggagccaggtcgacaatctatggacctggtagagctggtctctctgttcccaacgctcgccggacttgctggactgcaggtgccaccccgctgccctgtaccctccttccacgttgagctctgccgcgaaggcaagaacctgttgaaacattttcgattcagagaccttgaagaggacccatacctcccaggaaatccaagagagctgattgcttattctcaatatcccaggcccagtgacataccacagtggaatagcgataaaccctcacttaaagacattaagataatgggctattccatccggacaattgattacagatacacagtttgggtggggtttaacccagacgaattccttgcgaatttcagcgatattcatgccggagaactttattttgttgatagcgaccccctccaggaccacaacatgtacaacgactcacagggtggcgatctctttcagctcctgatgccgtga

SEQ ID NO: 188 - DNA, 1578 bp, Artificial Sequence
  source: /note=“Description of Artificial Sequence Synthetic polynucleotide”
tctgaaacccaggctaactctacgaccgacgcattgaatgttctgcttatcattgtagatgaccttcgccccagtttgggatgttatggcgataagctggtgcgctcacctaatattgatcagttggcaagccatagccttttgttccaaaatgcttttgctcagcaggctgtatgtgcaccgagtagagtttccttcctcaccggacgcagaccagacaccacaagactctacgactttaactcatactggagggtccacgctgggaatttcagtacgatcccgcagtatttcaaagaaaatggctacgttaccatgtccgtcggcaaggtgtttcaccccggcatctcatcaaatcatacagacgatagcccttattcttggtctttccctccttatcatccatccagcgaaaaatacgagaacactaaaacatgtagaggtccagatggagagctgcacgccaacctgctgtgccctgtggatgttctcgacgtacctgaaggcacccttccagacaaacagagcaccgaacaggccatccagcttctggagaagatgaagaccagcgcctcacctttcttcctcgccgtaggctaccacaaaccgcacatcccctttagatacccaaaggaatttcagaagctgtaccccctggaaaatataacattggctccagacccggaagtgcccgatgggttgccccccgtagcctataatccttggatggatattagacaacgggaagacgtccaggccctcaatatttctgtcccttacggaccaatccctgttgattttcagagaaagataagacagtcctattttgcaagtgtatcctaccttgacacccaggtcggccggctgttgtctgctctggacgacctgcaactcgctaacagtacaatcatagcctttactagcgaccacggatgggctctgggagaacatggagaatgggccaagtattctaacttcgatgtcgccacacacgtcccactcatattttacgttcctggtcgaaccgctagcctgcctgaagccggagaaaagctgtttccttatctcgaccctttcgattccgcaagccagttgatggaacccggccggcaatcaatggatctcgtggaactggtgtcactttttcctacactcgctggactcgctggccttcaagtccctccccgatgtcctgtcccatcatttcacgtagagctgtgtagagaagggaagaatctgctgaaacacttccggttccgggatcttgaagaagatccatatctcccaggcaaccctcgcgaactcatcgcttatagccagtatcctcggcccagtgacataccccagtggaattccgacaaaccatcacttaaagatatcaaaattatgggatactccattcgaaccatagactataggtacaccgtgtgggttggctttaatccagatgagtttttggcaaacttttcagacattcacgccggagagctgtattttgtggacagtgaccctctgcaggaccataatatgtacaacgattcacaaggcggcgacctcttccaactgctgatgccctga

SEQ ID NO: 189 - DNA, 1578 bp, Artificial Sequence
  source: /note=“Description of Artificial Sequence Synthetic polynucleotide”
tcacggcatgagcagctggaacaaatctcctccttgggaatcattatacatattgtgatcttgcaacgggtccgagtctacgaaatacagctcaccagcgtggatgtccgaaaagttcgcgaggaattcgtcaggattgaaccctacccacactgtgtagcgatagtcgatggtcctgatcgagtaccccataatcttgatgtctttgagggagggcttatcggagttccattgaggaatatcgctgggtcgcggatactgggaataggcaatcaactctcgcggattccctggcagataggggtcctcctcaaggtccctgaaccgaaagtgtttgaggaggtttttcccttcgcggcagagttccacatggaagctcggtacagggcatctagggggtacttgcaagcccgccaacccggcgagggtcggaaaaagggacaccaattctaccaagtccatggattgtctgcccggttccataagctggctcgccgagtcgaatggatcgagatagggaaaaagtttttcgcctgcctcgggaagcgaggccgttctacccggcacgtagaaaatcaggggcacgtgcgttgctacatcaaaattgctatactttgcccactctccatgctctcccaacgcccacccatggtccgacgtaaaggcgatgattgtggaatttgccagctgaaggtcatcaagcgcgctcagaagtcgacctacttgcgtatcgaggtaggacaccgacgcaaaatacgactgccgaatcttgcgttgaaaatcgactggaataggcccgtaggggactgagatgttgagtgcctgcacatcttccctctgcctgatatccatccagggattgtaggccacgggtggcagaccgtcggggacttccgggtccggtgccaaagtgatgttttccaaaggataaagtttctggaactccttcgggtagcggaaaggaatatggggcttgtgataccccacggcgaggaagaaaggcgacgcgcttgttttcatcttctccagcaactgaatcgcctgctccgttgactgcttgtcggggagcgttccctcgggcacgtccaagacatccaccggacacagcagattagcgtgcagctctccgtcgggtccgcgacaagttttcgtgttctcatacttctcgctcgaaggatggtagggaggaaacgaccacgagtagggcgaatcgtcggtgtgattcgaggagatgccggggtgaaagacctttcccacgctcattgtcacgtatccgttctctttaaagtactgtgggatagttgaaaagttacccgcgtggactctccagtagctgttgaagtcgtacagccgcgttgtgtcagggcgtcgcccggtcaagaatgagactcttgaaggtgcacagacagcctgctgcgcaaacgcattttggaaaagcagtgagtgtgaggccaactgatcgatgttcggcgagcggacgagcttatctccatagcagccaagcgacggccgcaaatcgtccacgatgatgagcaggacgttaagcgcatctgtagttgagttggcctgggtttcgct

SEQ ID NO: 190 - DNA, 1578 bp, Artificial Sequence
  source: /note=“Description of Artificial Sequence Synthetic polynucleotide”
tcaaggcatcaagagctggaacaaatctccgccttgggaatcgttgtacatattgtggtcttgcagtggatcggagtcgacgaaatacagctcaccagcgtgaatatcggaaaagtttgccaggaattcatcaggattgaagcccacccagacggtatatctgtagtcaatagtgcgaatagagtatcccataatcttaatgtccttgagggacggcttatcggaattccactgagggatgtcgctaggtctagggtattgggaataagcgatcagctcgcggggattaccgggcaagtaggggtcttcctccaggtcgcgaaaccgaaaatgtttaagcaagttttttccctccctgcacagctctacgtggaaagaaggaacggggcacctggggggaacttggagaccggccagaccggccagcgtgggaaagagggacacgagttcaaccaaatccatggattgtctaccgggctccatcaattggctagctgaatcaaaggggtccagataggggaacagtttctctcctgcttcgggcagactagcggttctaccggggacgtaaaagataaggggtacgtgtgtggcgacgtcgaaatttgaatatttggcccactcgccgtgttcgccgagcgcccacccgtggtcagaggtgaaggcaatgatggtgctgttagccaattgcaggtcgtcgagggcgctgagaagtctgccgacctgtgtgtcgaggtatgagacgctagcaaagtaggactggcggatctttctttgaaaatcaacgggaattggtccgtagggaactgagatattaagggcctgcacgtcctctctctgcctgatatccatccatgggttataggcgacagggggcaggccatcgggcacttcagggtcgggggccagggtgatgttttcaagaggatacaatttctggaattcttttggataccggaatggaatgtggggcttgtggtaaccgacggccaagaaaaaggggctggcagaggttttcattttctcaaggagctggatagcctgctcagtagattgtttatcagggagtgtcccctctggcacatcgagcacatcaactggacacaggagattggcgtgaagctcgccgtcgggacctctgcatgtctttgtattttcatatttttcacttgagggatgatatgggggaaaggaccagctgtatgggctatcgtcggtatggttggaagagatgcctggatggaacaccttgccaacgctcatggtgacgtatccgttctctttgaagtactgagggatggttgagaaattgccggcatgaacgcgccagtaactattaaaatcgtacagtcgggtcgtgtcagggcgtcgtccagtaagaaaagaaacgcgggatggtgcgcagacggcttgctgagcaaacgcgttctgaaaaagcagtgaatgggaagcaagctggtctatgttggggctgcgaaccagtttgtcgccgtagcagcccaaagagggtcgaagatcgtcaacaatgataaggaggacgttcaatgcatcagtggtggagttagcctgcgtctcact

SEQ ID NO: 191 - DNA, 1578 bp, Artificial Sequence
  source: /note=“Description of Artificial Sequence Synthetic polynucleotide”
tcaaggcatgagcaactgaaaaagatcacctccttgggaatcgttgtacatgttgtgatcttgaagggggtctgagtccacgaaatagagttctcctgcgtggatgtctgagaaattcgcgaggaattcatcgggattgaatccaacccatacggtatacctgtaatctatagtccggatgctgtagcccataatcttaatgtctttcaaagatggtttgtcagagttccactgtgggatgtcggagggtctggggtactgagaatatgcgatgagttctctaggattcccgggcaggtatgggtcttcctccaggtcgcgaaatcggaaatgtttcaggaggtttttcccttctctacaaagctcgacgtggaagctcggaacggggcatcgtggtggcacctgaaggccggcgagaccggcaagggtcggaaagagtgaaacgagctcgaccagatccatactttgtctaccgggttccatcagttgggatgcagagtcgaaggggtccagatatggaaagagtttttccccagcttctggcaaggacgcagtgcggccggggacatagaatatcaagggaacgtgggttgcgacatcgaaattggagtactttgcccattcgccgtgttctccgagtgcccatccatggtcgctggtaaaggcaattatggtagaattagcgagttgcagatcgtcaagggcgctgagcagtcttcctacttgtgtgtccaggtaactcacagaagcgaaataggattgtctaatcttgcgttgaaagtcgacaggtattgggccatatgggacgctgatgttaagagcctgcacgtcttcgcgctgcctgatgtccatccatgggttataggccactggggggagtccgtctgggacttctgggtctggtgccagtgtaatgttttcaagggggtacagcttctgaaattctttaggataccggaatggaatgtggggtttatgataacctacagccaggaaaaagggggatgcacttgttttcatcttttcgaggagctgaatggcctgctcggtagattgcttgtcggggagggtgccttccgggacgtcgagtacgtcaacagggcacaacaggtttgcgtgcagttcgccgtccggacctctacacgtcttggtattttcgtacttctctgaggaaggatggtatgggggaaaggaccaggagtagggagagtcgtctgtgtgattgcttgaaataccggggtgaaacaccttgcccacactcatagttacgtagccattttccttaaagtattgtggtattgtagaaaagttgccggcatgaacgcgccaatatgagttaaaatcgtacaggcgggtggtgtcgggcctgcgaccagtcaagaaggacactctactgggggcacaaactgcttgttgagcgaatgcgttttggaaaaggagggagtggcttgccagttggtcgatattcggagagcggaccagtttgtcaccgtaacagccgagtgaaggtctgaggtcatccactataatcagcaaaacattcaaggcgtccgtagtgctatttgcttgagtctctga

SEQ ID NO: 192 - DNA, 1578 bp, Artificial Sequence
  source: /note=“Description of Artificial Sequence Synthetic polynucleotide”
tcacggcatcaggagctgaaagagatcgccaccctgtgagtcgttgtacatgttgtggtcctggagggggtcgctatcaacaaaataaagttctccggcatgaatatcgctgaaattcgcaaggaattcgtctgggttaaaccccacccaaactgtgtatctgtaatcaattgtccggatggaatagcccattatcttaatgtctttaagtgagggtttatcgctattccactgtggtatgtcactgggcctgggatattgagaataagcaatcagctctcttggatttcctgggaggtatgggtcctcttcaaggtctctgaatcgaaaatgtttcaacaggttcttgccttcgcggcagagctcaacgtggaaggagggtacagggcagcggggtggcacctgcagtccagcaagtccggcgagcgttgggaacagagagaccagctctaccaggtccatagattgtcgacctggctccatcagttgggatgcactatcgaaaggatcgaggtaaggaaacagcttctcaccggcttctggcaggcttgcggttctacctggaacataaaatatgaggggcacgtgggtggcaacatcaaaattagagtatttggcccattctccgtgctcgccaagcgcccacccgtgatcgctagtgaaggcaatgattgtagagttagccaattgaaggtcgtcgagggcggacagcagtcttccgacctgggtgtcgagataagacacgcttgcaaagtaggactgcctgattttcctttggaaatcgacagggattgggccgtatggtactgagatgttgagtgcttgaacatcctcccgttgtctgatatccatccaggggttgtatgcaacagggggcaggccgtccgggacctcggggtcgggagcgagggtgatattttcaaggggatacagtttctgaaactctttagggtatctaaaaggaatgtggggcttgtggtaaccgactgcgaggaagaaaggtgaggcggacgttttcatcttttccagcagctgaatggcttgttcggtactctgcttgtctgggagagttccttcaggcacatcgagcacatctacggggcagagaaggtttgcgtgcaattctccgtcaggtccgcgacaagttttagtgttctcatatttttcactggaaggatggtacggtggaaatgaccaggaataggggctgtcatccgtatggttgctagaaattccgggatggaacactttgccgacgctcatggtaacgtatccgttttctttgaagtactgggggattgtagagaaattacctgcgtgcacgcgccagtaggaattgaagtcatacagcctagtagtatcggggcgtctgcctgtcagaaaggatactcttgaaggggcgcaaacggcctgttgtgcgaacgcgttttggaagaggagagaatgactcgccagctggtctatgttcggacttctaaccaatttatcgccatagcaaccaagtgagggtcgcagatcatccacgataataagcagaacgttcagcgcatcggtggttgagttcgcctgcgtttcact

SEQ ID NO: 193 - DNA, 1578 bp, Artificial Sequence
  source: /note=“Description of Artificial Sequence Synthetic polynucleotide”
tcagggcatcagcagttggaagaggtcgccgccttgtgaatcgttgtacatattatggtcctgcagagggtcactgtccacaaaatacagctctccggcgtgaatgtctgaaaagtttgccaaaaactcatctggattaaagccaacccacacggtgtacctatagtctatggttcgaatggagtatcccataattttgatatctttaagtgatggtttgtcggaattccactggggtatgtcactgggccgaggatactggctataagcgatgagttcgcgagggttgcctgggagatatggatcttcttcaagatcccggaaccggaagtgtttcagcagattcttcccttctctacacagctctacgtgaaatgatgggacaggacatcggggagggacttgaaggccagcgagtccagcgagtgtaggaaaaagtgacaccagttccacgagatccattgattgccggccgggttccatcaactggcttgcggaatcgaaagggtcgagataaggaaacagcttttctccggcttcaggcaggctagcggttcgaccaggaacgtaaaatatgagtgggacgtgtgtggcgacatcgaagttagaatacttggcccattctccatgttctcccagagcccatccgtggtcgctagtaaaggctatgattgtactgttagcgagttgcaggtcgtccagagcagacaacagccggccgacctgggtgtcaaggtaggatacacttgcaaaataggactgtcttatctttctctgaaaatcaacagggattggtccgtaagggacagaaatattgagggcctggacgtcttcccgttgtctaatatccatccaaggattataggctacggggggcaacccatcgggcacttccgggtctggagccaatgttatattttccagggggtacagcttctgaaattcctttgggtatctaaaggggatgtgcggtttgtggtagcctacggcgaggaagaaaggtgaggcgctggtcttcatcttctccagaagctggatggcctgttcggtgctctgtttgtctggaagggtgccttcaggtacgtcgagaacatccacagggcacagcaggttggcgtgcagctctccatctggacctctacatgttttagtgttctcgtatttttcgctggatggatgataaggagggaaagaccaagaataagggctatcgtctgtatgatttgatgagatgccggggtgaaacaccttgccgacggacatggtaacgtagccattttctttgaaatactgcgggatcgtactgaaattcccagcgtggaccctccagtatgagttaaagtcgtagagtcttgtggtgtctggtctgcgtccggtgaggaaggaaactctactcggtgcacatacagcctgctgagcaaaagcattttggaacaaaaggctatggcttgccaactgatcaatattaggtgagcgcaccagcttatcgccataacatcccaaactggggcgaaggtcatctacaatgataagcagaacattcaatgcgtcggtcgtagagttagcctgggtttcaga